(19) United States
(12) Patent Application Publication
(10) Pub. No.: US 2025/0136576 A1
(43) Pub. Date: May 1, 2025

(54) Heterocyclic Compounds and Uses Thereof

(71) Applicants: ACEA Therapeutics, Inc., San Diego, CA (US); Hangzhou ACEA Pharmaceutical Research Co., Ltd., HANGZHOU, Zhejiang Province (CN)

(72) Inventors: Long Mao, SAN DIEGO, CA (US); Wei Tang, HANGZHOU (CN); Xiaoying Zhang, HANGZHOU (CN); Rongda Xu, SAN DIEGO, CA (US); Yile Chen, HANGZHOU (CN); Xiao Xu, SAN DIEGO, CA (US); Changxu Xu, HANGZHOU (CN)

(73) Assignees: ACEA Therapeutics, Inc., San Diego, CA (US); Hangzhou ACEA Pharmaceutical Research Co., Ltd., HANGZHOU, Zhejiang Province (CN)

(21) Appl. No.: 18/578,240

(22) PCT Filed: Jul. 12, 2022
(86) PCT No.: PCT/US2022/036815

§ 371 Date: Jan. 10, 2024

(30) Foreign Application Priority Data
Jul. 13, 2021 (WO) PCT/CN2021/106076
Jun. 14, 2022 (WO) PCT/CN2022/098577

(51) Int. Cl. C07D 401/12; C07D 471/04; C07D 491/048

(52) U.S. Cl. CPC C07D 401/12; C07D 471/04; C07D 491/048

(57) ABSTRACT

The present disclosure relates to pharmaceutical compounds, compositions and methods, especially as they are related to compositions and methods comprising a compound of any one of Formulas (I), (II), (III), and (IV) as further described herein. The compounds are inhibitors of protein kinases associated with cancers, and are thus useful for the treatment and/or prevention of a tumor or cancer.

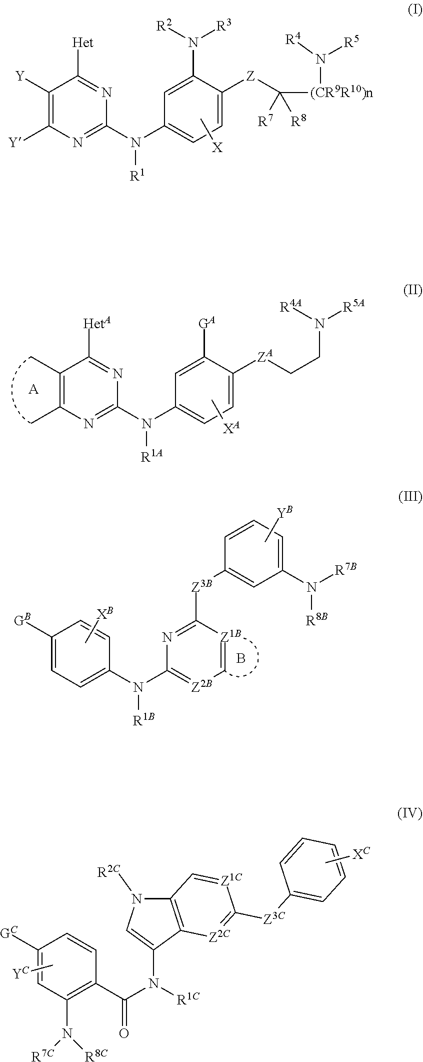

### CROSS-REFERENCE TO RELATED APPLICATIONS

This application claims priority to International Application No. PCT/CN2021/106076, filed on Jul. 13, 2021, and to International Application No. PCT/CN2022/098577, filed on Jun. 14, 2022, the disclosure of each of which is hereby incorporated by reference in its entirety.

### TECHNICAL FIELD

The present disclosure relates to compounds, pharmaceutical compositions and treatment methods, especially as they are related to compositions and methods for the treatment of a proliferation disorder, a cancer, or a neurodegenerative disease, or as they are related to modulation of immune response or sensitivity to pain, using compounds disclosed herein.

### BACKGROUND

Protein kinases are a group of enzymes that phosphorylate other proteins. Many protein kinases are involved in signal transduction within a cell or between the cell and its external environment: phosphorylation of signalling molecules turns key cellular functions ‘on or off’ and can drastically affect the survival or proliferation of the cell. Dysregulation of protein kinases, especially those involved in signaling and cell regulatory functions, is often associated with proliferative disorders such as cancers and neurodegenerative diseases such as Alzheimer's and Parkinson's. Dysregulation of protein kinases is also associated with modulation of immune response or sensitivity to pain. Indeed, many protein kinase mutations have been identified that promote or cause cancer, and many inhibitors of protein kinases have proven effective in treating such cancers. Acute myeloid leukemia (AML), for example, is often associated with mutations of FLT3 (fms-like tyrosine kinase 3), and inhibitors of the FLT3 kinase are known to be useful for treating some AML patients having such mutations. Daver, et al., Leukemia, vol. 33:299-312 (2019). Similarly, inhibitors of EGFR (epidermal growth factor receptor) kinase are useful to treat many types of cancer, including colorectal cancer. Li, et al., Gastroenterology Report vol. 8(3), 179-91 (2020). Likewise, inhibitors of neurotrophic tyrosine receptor kinase (NTRK) are reported to be useful in treatment of some non-small cell lung cancer patients. Tatematsu, et al., Molec. Clin. Oncology, vol. 2, 725-30 (2014). DYRK1/DYRK1A, DYRK1B, LRRK2, and MLK1/MAP3K9 inhibitors have been reported to be useful in the treatment of neurodegenerative diseases such as Alzheimer's disease and Parkinson's disease (Kargbo, R. Med Chem. Lett., vol. 11, 1795-1796 (2020); Abdel-Magid, A. Med Chem. Lett., vol. 10, 846-847 (2019); Craige, S. et al., Biochim. Et Biophys. Acta, vol 1862, 1581-1586 (2016)). c-SRC, FYN, LYN, SYK, JAK1, JAK3, GLK/MAP4K1 inhibitors have been reported to be useful for immune response modulation (Lowell, C. Cold Spring Harb Perspect Biol., vol 3, a002352 (2011); Szilveszter, K. et al., Front. Immunol. Vol. 10, Article 1862 (2019); Scapini, P. et al., Immunol. Rev. vol. 228, 23-40 (2009)). NTRK inhibitors have been reported to be useful in the treatment of pain in cancer patients (Drilon, A. Ann. Oncol. Vol. 30, viii23-viii30). Accordingly, new small-molecule inhibitors of protein kinases that are associated with cancers or neurodegenerative diseases, or capable of modulating immune responses or pain sensitivity in cancer patients are needed. The disclosure provides new compounds that inhibit one or more protein kinases known to be strongly associated with cancers, including FLT3, EGFR, VEGFR, ALK, NTRK, RET, ROS/ROS1, DYRK1 and CK2a kinases, and are thus useful for treating cancers associated with those protein kinases, including acute myeloid leukemia (AML), non-small cell lung cancer (NSCLC), hepatocellular carcinoma (HCC), thyroid cancer, mast cell tumors (MCT) and solid tumors with an NTRK gene fusion. Compounds of the disclosure often inhibit more than one cancer-associated kinase, and accordingly may be useful for treating cancer with reduced risk of mutations allowing the cancer to escape the compounds and treatments of the disclosure.

The disclosure also provides new compounds that inhibit one or more protein kinases known to be strongly associated with neurodegenerative diseases, including DYRK1/DYRK1A, DYRK1B, LRRK2, and MLK1/MAP3K9 kinases, and are thus useful for treating neurodegenerative diseases associated with those protein kinases, including Alzheimer's disease and Parkinson's disease. The disclosure further provides new compounds that inhibit one or more protein kinases known to be strongly associated with immune response modulation, including c-SRC, FYN, LYN, SYK, JAK1, JAK3, and GLK/MAP4K1. The disclosure further provides new compounds that inhibit one or more protein kinases known to be strongly associated with modulation of pain sensitivity in cancer patients, including NTRK.

### SUMMARY

In one aspect, the disclosure provides a heterocyclic compound having a structure according to Formula (I):

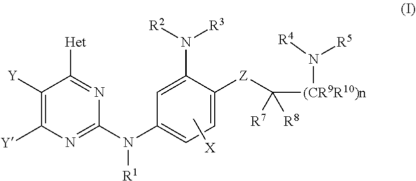

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR₆, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl; and
n is 1 to 4.

In another aspect, the disclosure provides a heterocyclic compound having a structure according to Formula (I):

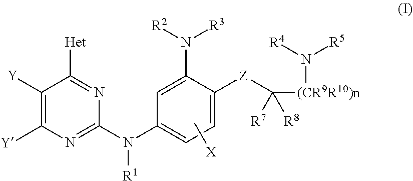

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl; and
n is 1 to 4.

In another aspect, the disclosure provides a heterocyclic compound having a structure according to Formula (IC):

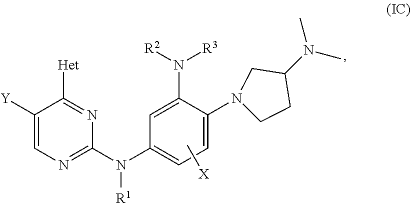

or an enantiomer, a mixture of enantiomers, a mixture of two or more diastereomers, a tautomer, a mixture of two or more tautomers, or an isotopic variant thereof; or a pharmaceutically acceptable salt thereof; wherein
X represents one or two optional substituents independently selected from halo, C₁-C₃ alkyl, C₁-C₃alkoxy, and C₁-C₃ haloalkyl;
Y is selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl; and
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl.

In another aspect, the disclosure provides a heterocyclic compound having a structure according to Formula (ID):

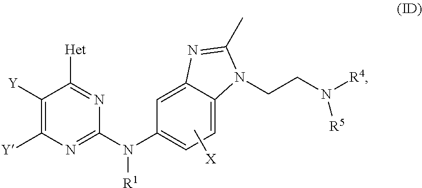

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R⁴ is selected from H and C₁-C₃ alkyl; and
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl.

In another aspect, the disclosure provides a heterocyclic compound having a structure according to Formula (IE):

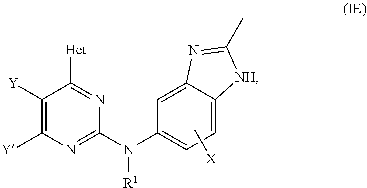

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole; and
R¹ is selected from H and C₁-C₃ alkyl.

In another aspect, the disclosure provides a heterocyclic compound having a structure according to Formula (II):

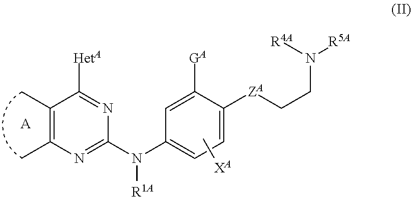

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Ring A is an optional 5-6 membered heterocyclic ring fused to the pyrimidine in Formula (II), comprising one or two heteroatoms selected from N and O as ring members,
   wherein Ring A can be aromatic or non-aromatic and is optionally substituted with one or two groups independently selected from halo, C₁-C₃ alkoxy, C₁-C₃ haloalkyl, and C₁-C₃ alkyl;
Alternatively, Ring A is absent, and pyrimidine is optionally substituted with one or two groups independently selected from halo, C₁-C₃ alkoxy, C₁-C₃ haloalkyl, and C₁-C₃ alkyl;
Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl;
X^{A} represents one or two optional substituents independently selected from halo, C₁-C₃ alkyl, C₁-C₃ alkoxy, and C₁-C₃ haloalkyl;
R^{1A} is selected from H and C₁-C₃ alkyl;
G^{A} is selected from —NR^{2A}R^{3A}, SO₂R^{7A}, halo, and C₁-C₃ haloalkyl;
   wherein R^{2A} is selected from H and C₁-C₃ alkyl, and
   R^{3A} is selected from H, C₁-C₃ alkyl, C₂-C₄ alkenyl, C₁-C₃ haloalkyl, —SO₂R^{7A}, and —C(O)—R^{11A},
   wherein R^{11A} is selected from H, C₁-C₃ alkyl, C₂-C₄ alkenyl, C₁-C₃ alkoxy, and C₁-C₃ haloalkyl;
Z^{A} is selected from O, NR^{6A}, C(═O), SO₂, C(═O)NR^{6A}, SO₂NR^{6A}, and (CH₂)₁₋₂;
R^{4A} is selected from H and C₁-C₃ alkyl;
R^{5A} is selected from H, C₁-C₃ alkyl, C₁-C₃ haloalkyl, —C(O)—R^{12A}, and —SO₂R^{7A},
   wherein R^{12A} is selected from H, C₁-C₃ alkyl, C₁-C₃ alkoxy, and C₁-C₃ haloalkyl;
R^{6A} is selected from H and C₁-C₃ alkyl; and
each R^{7A} is independently C₁-C₃ alkyl.

In another aspect, the disclosure provides a heterocyclic compound having a structure according to Formula (III):

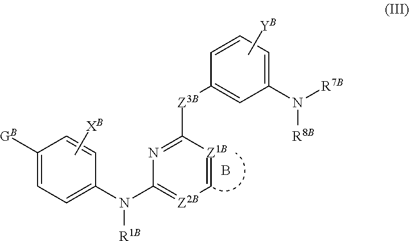

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Ring B is an optional 5 membered heteroaromatic ring fused to the ring containing Z^{2B} in Formula (III), comprising N or O as a ring member,
wherein Ring B is optionally substituted with one or two groups independently selected from halo, C₁-C₃ alkoxy, C₁-C₃ haloalkyl, C₁-C₃ alkyl, and —C(O)—R^{10B};
Z^{1B} is N when ring B is absent, and Z^{1B} is C when ring B is present;
Z^{2B} is N when ring B is present, and Z^{2B} is CR^{2B} when ring B is absent;
Z^{3B} is NR^{3B} or O;
G^{B} is a group of the formula —NR^{4B}—(CR^{1B})₂₋₃—NR^{5B}R^{6B} or G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B};
X^{B} represents one or two optional substituents independently selected from halo, C₁-C₃ alkyl, C₁-C₃ alkoxy, and C₁-C₃ haloalkyl;
Y^{B} represents one or two optional substituents independently selected from halo, C₁-C₃ alkyl, C₁-C₃ alkoxy, and C₁-C₃ haloalkyl;
R^{1B} is selected from H and C₁-C₃ alkyl;
R^{2B} is selected from H, halo, C₁-C₃ alkyl, and C₁-C₃ haloalkyl;
R^{3B} is selected from H and C₁-C₃ alkyl;
R^{4B} is selected from H, C₁-C₃ alkyl, and —C(O)—R^{10B};
R^{5B} is selected from H and C₁-C₃ alkyl;
R^{6B} is selected from H, C₁-C₃ alkyl, and —C(O)—R^{10B};
R^{7B} is selected from H and C₁-C₃ alkyl;
R^{8B} is selected from H, C₁-C₃ alkyl and —C(O)—R^{10B}; and
each R^{10B} is independently selected from H, C₁-C₃ alkyl, C₁-C₃ alkoxy, and C₁-C₃ haloalkyl.

In another aspect, the disclosure provides a heterocyclic compound having a structure according to Formula (IV):

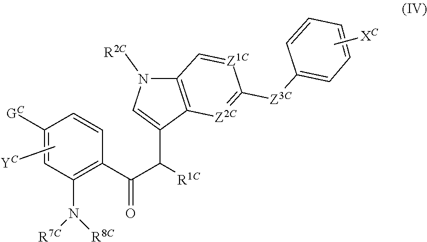

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Z^{1C} and Z^{2C} are independently selected from N and CH;
Z^{3C} is selected from O, CH₂, and NR^{3C};
G^{C} is a group of the formula —NR^{4C}—(CR^{2C})₂₋₃—NR^{5C}R^{6C} or G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C};
X^{C} represents one or two optional substituents independently selected from halo, C₁-C₃ alkyl, C₁-C₃ alkoxy, and C₁-C₃ haloalkyl;
Y^{C} represents one or two optional substituents independently selected from halo, C₁-C₃ alkyl, C₁-C₃ alkoxy, and C₁-C₃ haloalkyl;
R^{1C} is selected from H and C₁-C₃ alkyl;
R^{2C} is selected from H and C₁-C₃ alkyl;
R^{3C} is selected from H and C₁-C₃ alkyl;
R^{4C} is selected from H, C₁-C₃ alkyl, and —C(O)—R^{10C};
R^{5C} is selected from H and C₁-C₃ alkyl;
R^{6C} is selected from H, C₁-C₃ alkyl, and —C(O)—R^{10C};
R^{7C} is selected from H, C₁-C₄ alkyl optionally substituted with C₁-C₃ alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl;
R^{8C} is selected from H, C₁-C₃ alkyl and —C(O)—R^{10C};
each R^{10C} is independently selected from H, C₁-C₃ alkyl, C₁-C₃ alkoxy, and C₁-C₃ haloalkyl.

The compounds of Formulas (I)-(IV) and subformulas thereof are described in more detail below.

The compound of any one of Formulas (I)-(IV) as described above can be used for any suitable purpose. In some embodiments, the compounds described above are used in therapy or for manufacture of a medicament for treating the conditions mentioned herein, particularly for the treatment of a cancer associated with a kinase selected from FLT3, EGFR, VEGFR, ALK, NTRK, RET, ROS/ROS1, DYRK1 and CK2a.

In still another aspect, the present disclosure provides a pharmaceutical composition comprising a compound of any one of Formulas (I)-(IV) as described herein admixed with at least one pharmaceutically acceptable carrier or excipient. Such pharmaceutical compositions are useful for treating cancers such as those disclosed herein.

In yet another aspect, the present disclosure provides a method for treating and/or preventing a proliferation disorder, a cancer, or a tumor which comprises administering to a subject in need thereof an effective amount of a compound of any one of Formulas (I)-(IV) as described herein, or a pharmaceutical composition comprising such compounds. In some embodiments, the methods are useful for treating cancers associated with protein kinases, including FLT3, EGFR, VEGFR, ALK, NTRK, RET, ROS/ROS1, DYRK1 and CK2a kinases, for treating acute myeloid leukemia (AML), non-small cell lung cancer (NSCLC), hepatocellular carcinoma (HCC), thyroid cancer, mast cell tumors (MCT) and solid tumors with an NTRK gene fusion among others.

In yet another aspect, the present disclosure provides for a use of a compound of any one of Formulas (I)-(IV) as described above for the manufacture of a medicament. Typically, the medicament is for treatment of a condition described herein such as cancers or similar proliferative disorders. In some embodiments, the medicament is useful for treating cancers associated with protein kinases, including FLT3, EGFR, VEGFR, ALK, NTRK, RET, ROS/ROS1, DYRK1 and CK2a kinases, e.g., for treating acute myeloid leukemia (AML), non-small cell lung cancer (NSCLC), hepatocellular carcinoma (HCC), thyroid cancer, mast cell tumors (MCT) and solid tumors with an NTRK gene fusion among others.

In yet another aspect, the present disclosure provides a pharmaceutical combination for treating and/or preventing a proliferation disorder, a cancer, or a tumor in a subject, which combination comprises an effective amount of a compound of any one of Formulas (I)-(IV) as described above, or a pharmaceutically acceptable salt thereof, and an effective amount of a second prophylactic or therapeutic agent for treating and/or preventing a proliferation disorder in a subject in need of such therapy. In some embodiments, the medicament is useful for treating cancers associated with protein kinases, including FLT3, EGFR, VEGFR, ALK, NTRK, RET, ROS/ROS1, DYRK1 and CK2a kinases, e.g., for treating acute myeloid leukemia (AML), non-small cell lung cancer (NSCLC), hepatocellular carcinoma (HCC), thyroid cancer, mast cell tumors (MCT) and solid tumors with an NTRK gene fusion among others. The second therapeutic agent can be a small molecule or a biologic. It can be a kinase inhibitor, cytotoxin, or a checkpoint (PD-1, PD-L1) inhibitor.

In yet another aspect, the present disclosure provides a method for treating and/or preventing a proliferation disorder, a cancer, or a tumor in a subject, which methods comprises administering to a subject in need thereof an effective amount of the pharmaceutical combination described above. In some embodiments, the proliferation condition is selected from the group consisting of sarcoma, epidermoid cancer, fibrosarcoma, cervical cancer, gastric carcinoma, skin cancer, leukemia including acute myeloid leukemia, lymphoma including non-Hodgkin lymphoma, lung cancer, non-small cell lung cancer, colon cancer, CNS cancer, melanoma, ovarian cancer, renal cancer, thyroid cancer, prostate cancer, breast cancer, liver cancer, head and neck cancers, pancreatic cancer, mast cell tumors, and solid tumors with a NTRK gene fusion.

In yet another aspect, the present disclosure provides a method for inhibiting an activity of a cancer-associated kinase, including FLT3, ALK, EGFR, VEGFR, NTRK, RET, ROS/ROS1, DYRK1, and CK2a kinases, and the respective pathways, in a cell or subject, which methods comprises administering to a cell or subject in need thereof an effective amount of a compound of any one of Formulas (I)-(IV) as described herein, or a pharmaceutical composition comprising such compound, or a combination described herein containing such compound.

### DETAILED DESCRIPTION OF THE INVENTION

### General Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

The term “alkyl” as used herein refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tertiary butyl, pentyl, isopentyl, hexyl, cyclopropylmethyl, etc.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

Alkyl groups can be unsubstituted, or they can be substituted to the extent that such substitution makes sense chemically. Typical substituents include, but are not limited to, halo, ═O, ═N—CN, ═N—OR^{a}, ═NR^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C1-C8 alkyl, C2-C8 heteroalkyl, C3-C8 heterocyclyl, C4-C10 heterocyclyclalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R^{a} is optionally substituted with halo, ═O, ═N—CN, ═N—OR^{b}, ═NR^{b}, OR^{b}, NR^{b}₂, SR^{b}, SO₂R^{b}, SO₂NR^{b}₂, NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C1-C8 alkyl, C2-C8 heteroalkyl, C3-C8 heterocyclyl, C4-C10 heterocyclyclalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where a substituent group contains two R^{a} or R^{b} groups on the same or adjacent atoms (e.g., —NR^{b}2, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “alkenyl” as used herein refers to an alkyl as defined above having at least two carbon atoms and at least one carbon-carbon double bond. Thus, particularly contemplated alkenyl groups include straight, branched, or cyclic alkenyl groups having two to ten carbon atoms (e.g., ethenyl, propenyl, butenyl, pentenyl, etc.) or 5-10 atoms for cyclic alkenyl groups. Alkenyl groups are optionally substituted by groups suitable for alkyl groups as set forth herein.

Similarly, the term “alkynyl” as used herein refers to an alkyl or alkenyl as defined above and having at least two (preferably three) carbon atoms and at least one carbon-carbon triple bond. Especially contemplated alkynyls include straight, branched, or cyclic alkynes having two to ten total carbon atoms (e.g., ethynyl, propynyl, butynyl, cyclopropylethynyl, etc.). Alkynyl groups are optionally substituted by groups suitable for alkyl groups as set forth herein.

The term “cycloalkyl” as used herein refers to a cyclic alkane (i.e., in which a chain of carbon atoms of a hydrocarbon forms a ring), preferably including three to eight carbon atoms. Thus, exemplary cycloalkanes include cyclopropyl, cyclobutyl, cyclopentyl, cyclohexyl, cycloheptyl, and cyclooctyl. Cycloalkyls also include one or two double bonds, which form the “cycloalkenyl” groups. Cycloalkyl groups are optionally substituted by groups suitable for alkyl groups as set forth herein.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group, and are thus termed “fused aryl” or “fused aromatic”.

Aromatic groups containing one or more heteroatoms (typically N, O or S) as ring members can be referred to as heteroaryl or heteroaromatic groups. Typical heteroaromatic groups include monocyclic C5-C6 aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle”, “cycloheteroalkyl”, and “heterocyclic moieties” are used interchangeably herein and refer to any compound in which a plurality of atoms form a ring via a plurality of covalent bonds, wherein the ring includes at least one atom other than a carbon atom as a ring member. Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, piperazine, thiazole, tetrazole etc.). Typically these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles, and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

Heterocyclic groups that are not aromatic can be substituted with groups suitable for alkyl group substituents, as set forth above.

Aryl and heteroaryl groups can be substituted where permitted. Suitable substituents include, but are not limited to, halo, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C1-C8 alkyl, C2-C8 heteroalkyl, C3-C8 heterocyclyl, C4-C10 heterocyclyclalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R^{a} is optionally substituted with halo, ═O, ═N—CN, ═N—OR^{b}, ═NR^{b}, OR^{b}, NR^{b}₂, SR^{b}, SO₂R^{b}, SO₂NR^{b}₂, NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C1-C8 alkyl, C2-C8 heteroalkyl, C3-C8 heterocyclyl, C4-C10 heterocyclyclalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where a substituent group contains two R^{a} or R^{b} groups on the same or adjacent atoms (e.g., —NR^{b}2, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “alkoxy” as used herein refers to a hydrocarbon group connected through an oxygen atom, e.g., —O-Hc, wherein the hydrocarbon portion Hc may have any number of carbon atoms, typically 1-10 carbon atoms, may further include a double or triple bond and may include one or two oxygen, sulfur or nitrogen atoms in the alkyl chains, and can be substituted with aryl, heteroaryl, cycloalkyl, and/or heterocyclyl groups. For example, suitable alkoxy groups include methoxy, ethoxy, propyloxy, isopropoxy, methoxyethoxy, benzyloxy, allyloxy, and the like. Similarly, the term “alkylthio” refers to alkylsulfides of the general formula —S-Hc, wherein the hydrocarbon portion Hc is as described for alkoxy groups. For example, contemplated alkylthio groups include methylthio, ethylthio, isopropylthio, methoxyethylthio, benzylthio, allylthio, and the like.

The term ‘amino’ as used herein refers to the group —NH₂. The term “alkylamino” refers to amino groups where one or both hydrogen atoms are replaced by a hydrocarbon group Hc as described above, wherein the amino nitrogen “N” can be substituted by one or two Hc groups as set forth for alkoxy groups described above. Exemplary alkylamino groups include methylamino, dimethylamino, ethylamino, diethylamino, etc. Also, the term “substituted amino” refers to amino groups where one or both hydrogen atoms are replaced by a hydrocarbon group Hc as described above, wherein the amino nitrogen “N” can be substituted by one or two Hc groups as set forth for alkoxy groups described above.

The term ‘acyl’ as used herein refers to a group of the formula —C(═O)-D, where D represents an alkyl, alkenyl, alkynyl, cycloalkyl, aryl, heteroaryl, or heterocycle as described above. Typical examples are groups wherein D is a C₁-C₁₀ alkyl, C₂-C₁₀ alkenyl or alkynyl, or phenyl, each of which is optionally substituted. In some embodiments, D can be H, Me, Et, isopropyl, propyl, butyl, C₁-C₄ alkyl substituted with —OH, —OMe, or NH₂, phenyl, halophenyl, alkylphenyl, and the like.

The term “aryloxy” as used herein refers to an aryl group connecting to an oxygen atom, wherein the aryl group may be further substituted. For example suitable aryloxy groups include phenyloxy, etc. Similarly, the term “arylthio” as used herein refers to an aryl group connecting to a sulfur atom, wherein the aryl group may be further substituted. For example suitable arylthio groups include phenylthio, etc.

The hydrocarbon portion of each alkoxy, alkylthio, alkylamino, and aryloxy, etc. can be substituted as appropriate for the relevant hydrocarbon moiety.

The term “halogen” as used herein refers to fluorine, chlorine, bromine and iodine. Where present as a substituent group, halogen or halo typically refers to F or Cl or Br, more typically F or Cl.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

The term “haloalkoxy” refers to the group alkyl-O— wherein one or more hydrogen atoms on the alkyl group have been substituted with a halo group and include, by way of examples, groups such as trifluoromethoxy, and the like.

The term “sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein each alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

The term “sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein

The term “aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

The term “acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR20C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “aminocarbonylamino” refers to the group —NR²⁰C(O)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

It should further be recognized that all of the above-defined groups may further be substituted with one or more substituents, which may in turn be substituted with hydroxy, amino, cyano, C₁-C₄ alkyl, halo, or C₁-C₄ haloalkyl. For example, a hydrogen atom in an alkyl or aryl can be replaced by an amino, halo or C₁₋₄ haloalkyl or alkyl group.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃+), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, PO₃H, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (3H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I) It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of skill.

The term “isotopic enrichment” refers to the percentage of incorporation of a less prevalent isotope (e.g., D for hydrogen) of an element at a given position in a molecule in the place of a more prevalent isotope (e.g., ¹H for hydrogen) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent hydrogen isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The phrase “an enantiomer, a mixture of enantiomers, a mixture of two or more diastereomers, a tautomer, a mixture of two or more tautomers, or isotopic variant thereof; or a pharmaceutically acceptable salt, solvate, hydrate, or prodrug thereof” has the same meaning as the phrase “an enantiomer, a mixture of enantiomers, a mixture of two or more diastereomers, a tautomer, a mixture of two or more tautomers, or isotopic variant of the compound referenced therein; or a pharmaceutically acceptable salt, solvate, hydrate, or prodrug of the compound referenced therein, or a pharmaceutically acceptable salt, solvate, hydrate, or prodrug of an enantiomer, a mixture of enantiomers, a mixture of two or more diastereomers, a tautomer, a mixture of two or more tautomers, or isotopic variant of the compound referenced therein.”

The compounds and compositions described herein can be administered to a subject in need of treatment for a cell proliferation disorder such as cancer, particularly cancers selected from AML, leukemia, lymphoma, lung cancer including non-small cell lung cancer, colon and colorectal cancer, CNS cancer, melanoma, ovarian cancer, renal cancer, prostate cancer, breast cancer, head and neck cancers, and pancreatic cancer. The subject is typically a mammal diagnosed as being in need of treatment for one or more of such proliferative disorders, and frequently the subject is a human. The methods comprise administering an effective amount of at least one compound of the disclosure; optionally the compound may be administered in combination with one or more additional therapeutic agents, particularly therapeutic agents known to be useful for treating the cancer or proliferative disorder afflicting the particular subject.

The following enumerated embodiments are representative of some aspects of the disclosure.
1. A compound of Formula (I):

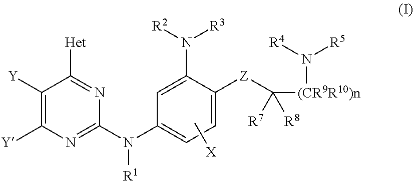

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl;
n is 1 to 4.
2. A compound of Formula (I):

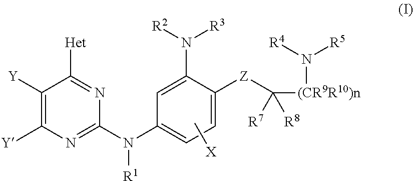

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl; and
n is 1 to 4.
3. A compound of Formula (I):

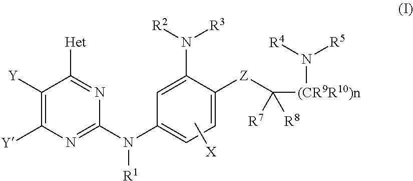

wherein:
X represents one or two optional substituents selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl;
n is 1 to 4;
   when Z is NR⁶, R⁹ can optionally be linked to R⁶ by one or two carbon atoms to form a 5 or 6 membered ring; or
   when Z is NR⁶, R⁶ can optionally cyclize with R³ to from an imidazole ring fused to the phenyl that is substituted with Z, where the imidazole group is optionally substituted with C₁-C₃ alkyl;
or a pharmaceutically acceptable salt thereof.
4. The compound of any one of embodiments 1-3, or a pharmaceutically acceptable salt thereof, wherein Y′ is H, or a pharmaceutically acceptable salt thereof.
5. The compound of any one of the preceding embodiments, or a pharmaceutically acceptable salt thereof, wherein Y is selected from F, CF₃, OMe and Cl.
6. The compound of any one of the preceding embodiments or a pharmaceutically acceptable salt thereof, wherein Het is selected from

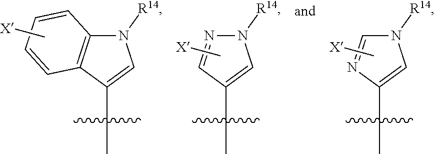

wherein each X′ represents up to two optional substituents independently selected from halo, C₁-C₃ alkyl, and C₁-C₃ alkoxy, and each R¹⁴ represents H, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy.
7. The compound of any one of the preceding embodiments or a pharmaceutically acceptable salt thereof, wherein Z is NR⁶.
8. The compound of any one of the preceding embodiments or a pharmaceutically acceptable salt thereof, wherein n is 1.
9. The compound of any one of the preceding embodiments or a pharmaceutically acceptable salt thereof, wherein R⁷, R⁸, R⁹ and R¹⁰ each represent H.
10. The compound of any one of the preceding embodiments or a pharmaceutically acceptable salt thereof, wherein R¹ is H.
11. The compound of any one of the preceding embodiments or a pharmaceutically acceptable salt thereof, wherein R² is H.
12. The compound of any one of the preceding embodiments, which is a compound of Formula (IA):

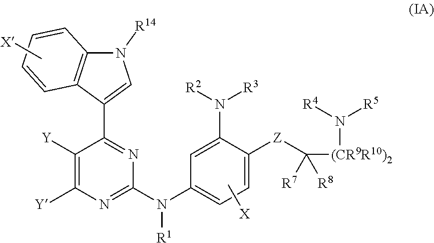

or an enantiomer, a mixture of enantiomers, a mixture of two or more diastereomers, a tautomer, a mixture of two or more tautomers, or an isotopic variant thereof; or a pharmaceutically acceptable salt thereof; wherein R¹⁴ is selected from H, methyl, ethyl, isopropyl, cyclopropyl and —C(═O)—R* where R* is H, C₁₋₃ haloalkyl, or C₁₋₄ alkyl optionally substituted with OH, CN, or C₁₋₃ alkoxy.
13. The compound of any one of the preceding embodiments, which is of the formula (IB):

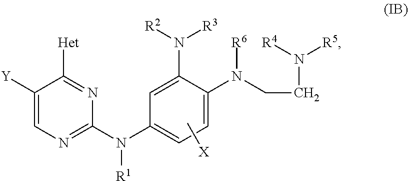

or an enantiomer, a mixture of enantiomers, a mixture of two or more diastereomers, a tautomer, a mixture of two or more tautomers, or an isotopic variant thereof; or a pharmaceutically acceptable salt thereof; wherein R⁴, R⁵, and R⁶ are each independently selected from methyl and ethyl.
14. A compound of Formula (IC):

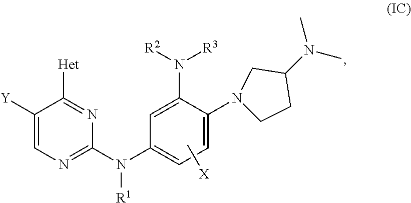

or an enantiomer, a mixture of enantiomers, a mixture of two or more diastereomers, a tautomer, a mixture of two or more tautomers, or an isotopic variant thereof; or a pharmaceutically acceptable salt thereof; wherein
   X represents one or two optional substituents independently selected from halo, C₁-C₃ alkyl, C₁-C₃alkoxy, and C₁-C₃ haloalkyl;
Y is selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl; and
R³ is selected from H, C₁-C₃ alkyl, C₁-C₃ haloalkyl, and —C(O)—R¹¹, wherein R¹¹ is selected from H, C₁-C₃ alkyl, C₁-C₃ alkoxy, and C₁-C₃ haloalkyl.
15. A compound of Formula (ID):

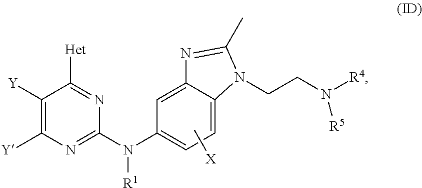

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R⁴ is selected from H and C₁-C₃ alkyl; and
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl.
16. A compound of Formula (IE):

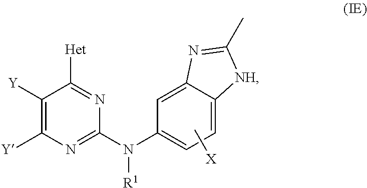

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole; and
R¹ is selected from H and C₁-C₃ alkyl.
17. The compound of any one of embodiments 1-6 or a pharmaceutically acceptable salt thereof, wherein Z is O.
18. A compound of Formula (II):

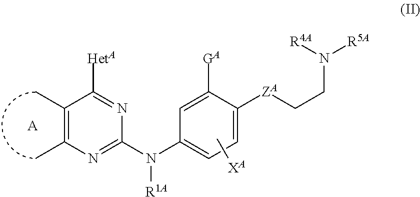

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Ring A is an optional 5-6 membered heterocyclic ring fused to the pyrimidine in Formula (II), comprising one or two heteroatoms selected from N and O as ring members, wherein Ring A can be aromatic or non-aromatic and is optionally substituted with one or two groups independently selected from halo, C₁-C₃ alkoxy, C₁-C₃ haloalkyl, and C₁-C₃ alkyl;
Alternatively, Ring A is absent, and pyrimidine is optionally substituted with one or two groups independently selected from halo, C₁-C₃ alkoxy, C₁-C₃ haloalkyl, and C₁-C₃ alkyl;
Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl;
X^{A} represents one or two optional substituents independently selected from halo, C₁-C₃ alkyl, C₁-C₃ alkoxy, and C₁-C₃ haloalkyl;
R^{1A} is selected from H and C₁-C₃ alkyl;
G^{A} is selected from —NR^{2A}R^{3A}, SO₂R^{7A}, halo, and C₁-C₃ haloalkyl;
   wherein R^{2A} is selected from H and C₁-C₃ alkyl, and
   R^{3A} is selected from H, C₁-C₃ alkyl, C₂-C₄ alkenyl, C₁-C₃ haloalkyl, —SO₂R^{7A}, and —C(O)—R^{11A},
   wherein R^{11A} is selected from H, C₁-C₃ alkyl, C₂-C₄ alkenyl, C₁-C₃ alkoxy, and C₁-C₃ haloalkyl;
Z^{A} is selected from O, NR^{6A}, C(═O), SO₂, C(═O)NR^{6A}, SO₂NR^{6A}, and (CH₂)₁₋₂;
R^{4A} is selected from H and C₁-C₃ alkyl;
R^{5A} is selected from H, C₁-C₃ alkyl, C₁-C₃ haloalkyl, —C(O)—R^{12A}, and —SO₂R^{7A}, wherein R^{12A} is selected from H, C₁-C₃ alkyl, C₁-C₃ alkoxy, and C₁-C₃ haloalkyl;
R^{6A} is selected from H and C₁-C₃ alkyl; and
each R^{7A} is independently C₁-C₃ alkyl.
19. A compound of Formula (II):

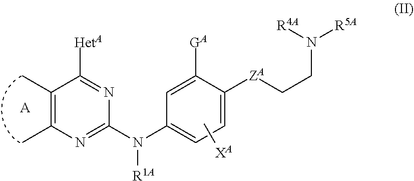

wherein:
Ring A is a 5-6 membered heterocyclic ring fused to the pyrimidine in Formula (II), comprising one or two heteroatoms selected from N and O as ring members,
   wherein Ring A can be aromatic or non-aromatic and is optionally substituted with one or two groups selected from halo, C₁-C₃ alkoxy, C₁-C₃ haloalkyl, and C₁-C₃ alkyl;
Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl;
X^{A} represents one or two optional substituents selected from halo, C₁-C₃ alkyl, C₁-C₃ alkoxy, and C₁-C₃ haloalkyl;
R^{1A} is selected from H and C₁-C₃ alkyl;
G^{A} is selected from —NR^{2A}R^{3A}, SO₂R^{7A}, halo, and C₁-C₃ haloalkyl;
   wherein R^{2A} is selected from H and C₁-C₃ alkyl, and
   R^{3A} is selected from H, C₁-C₃ alkyl, C₂-C₄ alkenyl, C₁-C₃ haloalkyl, —SO₂R^{7A}, and —C(O)—R^{11A},
   wherein R^{11A} is selected from H, C₁-C₃ alkyl, C₂-C₄ alkenyl, C₁-C₃ alkoxy, and C₁-C₃ haloalkyl;
Z^{A} is selected from O, NR^{6A}, C(═O), SO₂, C(═O)NR^{6A}, SO₂NR^{6A}, and (CH₂)₁₋₂;
R^{4A} is selected from H and C₁-C₃ alkyl;
R^{5A} is selected from H, C₁-C₃ alkyl, C₁-C₃ haloalkyl, —C(O)—R^{12A}, and —SO₂R^{7A},
   wherein R^{12A} is selected from H, C₁-C₃ alkyl, C₁-C₃ alkoxy, and C₁-C₃ haloalkyl;
R^{6A} is selected from H and C₁-C₃ alkyl; and
each R^{7A} is independently C₁-C₃ alkyl;
   or a pharmaceutically acceptable salt thereof.
20. The compound of any one of embodiments 18-19 or a pharmaceutically acceptable salt thereof, wherein R^{1A} is H.
21. The compound of any one of embodiments 18-20 or a pharmaceutically acceptable salt thereof, wherein Het^{A} represents an optionally substituted indole.
22. The compound of any one of embodiments 18-21 or a pharmaceutically acceptable salt thereof, wherein Z^{A} is NR^{6A}.
23. The compound of any one of embodiments 18-21 or a pharmaceutically acceptable salt thereof, wherein Z^{A} is O.
24. The compound of any one of embodiments 18-23 or a pharmaceutically acceptable salt thereof, wherein G^{A} is —NR^{2A}R^{3A}.
25. The compound of any one of embodiments 18-24, which is a compound of Formula (IIA):

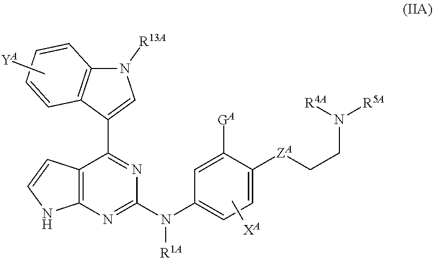

or an enantiomer, a mixture of enantiomers, a mixture of two or more diastereomers, a tautomer, a mixture of two or more tautomers, or an isotopic variant thereof; or a pharmaceutically acceptable salt thereof; wherein:
R^{13A} is selected from H and C₁-C₃ alkyl; and
Y^{A} represents one or two optional substituents independently selected from halo, C₁-C₃ alkyl, C₁-C₃ alkoxy, and C₁-C₃ haloalkyl.
26. The compound of any one of embodiments 18-25 or a pharmaceutically acceptable salt thereof, wherein the group —Z^{A} (CH₂)₂—NR^{4A}R^{5A} represents —NMe-CH₂CH₂—NMe₂ or -NEt-CH₂CH₂—NMe₂.
27. The compound of any one of embodiments 18-26 or a pharmaceutically acceptable salt thereof, wherein Ring A is selected from pyrrole and furan.
28. A compound of Formula (III):

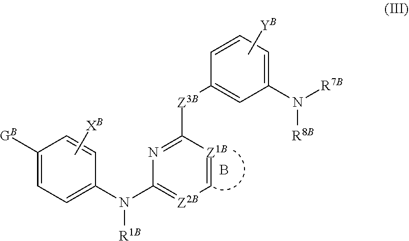

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Ring B is an optional 5 membered heteroaromatic ring fused to the ring containing Z^{2B} in Formula (III), comprising N or O as a ring member,
wherein Ring B is optionally substituted with one or two groups independently selected from halo, C₁-C₃ alkoxy, C₁-C₃ haloalkyl, C₁-C₃ alkyl, and —C(O)—R^{10B};
Z^{1B} is N when ring B is absent, and Z^{1B} is C when ring B is present;
Z^{2B} is N when ring B is present, and Z^{2B} is CR^{2B} when ring B is absent;
Z^{3B} is NR^{3B} or O;
G^{B} is a group of the formula —NR^{4B}—(CR^{1B})₂₋₃—NR^{5B}R^{6B} or G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B};
X^{B} represents one or two optional substituents independently selected from halo, C₁-C₃ alkyl, C₁-C₃ alkoxy, and C₁-C₃ haloalkyl;
Y^{B} represents one or two optional substituents independently selected from halo, C₁-C₃ alkyl, C₁-C₃ alkoxy, and C₁-C₃ haloalkyl;
R^{1B} is selected from H and C₁-C₃ alkyl;
R^{2B} is selected from H, halo, C₁-C₃ alkyl, and C₁-C₃ haloalkyl;
R^{3B} is selected from H and C₁-C₃ alkyl;
R^{4B} is selected from H, C₁-C₃ alkyl, and —C(O)—R^{10B};
hR^{5B} is selected from H and C₁-C₃ alkyl;
R^{6B} is selected from H, C₁-C₃ alkyl, and —C(O)—R^{10B};
R^{7B} is selected from H and C₁-C₃ alkyl;
R^{8B} is selected from H, C₁-C₃ alkyl and —C(O)—R^{10B};
each R^{10B} is independently selected from H, C₁-C₃ alkyl, C₁-C₃ alkoxy, and C₁-C₃ haloalkyl.
29. A compound of Formula (III):

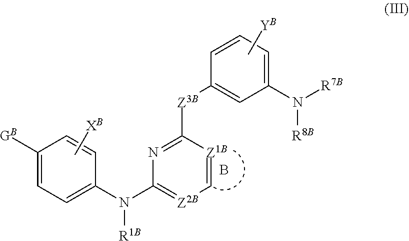

wherein:
Ring B is an optional 5 membered heteroaromatic ring fused to the ring containing Z^{2B} in Formula (III), comprising N or O as a ring member,
wherein Ring B is optionally substituted with one or two groups selected from halo, C₁-C₃ alkoxy, C₁-C₃ haloalkyl, C₁-C₃ alkyl, and —C(O)—R^{10B};
Z^{1B} is N when ring B is absent, and Z^{1B} is C when ring B is present;
Z^{2B} is N when ring B is present, and Z^{2B} is CR^{2B} when ring B is absent;
Z^{3B} is NR^{3B} or O;
G^{B} is a group of the formula —NR^{4B}—(CR₂)₂₋₃—NR^{5B}R^{6B} or G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B};
X^{B} represents one or two optional substituents selected from halo, C₁-C₃ alkyl, C₁-C₃ alkoxy, and C₁-C₃ haloalkyl;
Y^{B} represents one or two optional substituents selected from halo, C₁-C₃ alkyl, C₁-C₃ alkoxy, and C₁-C₃ haloalkyl;
R^{1B} is selected from H and C₁-C₃ alkyl;
R^{2B} is selected from H, halo, C₁-C₃ alkyl, and C₁-C₃ haloalkyl;
R^{3B} is selected from H and C₁-C₃ alkyl;
R^{4B} is selected from H, C₁-C₃ alkyl, and —C(O)—R^{10B};
R^{5B} is selected from H and C₁-C₃ alkyl;
R^{6B} is selected from H, C₁-C₃ alkyl, and —C(O)—R^{10B};
R^{7B} is selected from H and C₁-C₃ alkyl;
R^{8B} is selected from H, C₁-C₃ alkyl and —C(O)—R^{10B};
each R^{10B} is independently selected from H, C₁-C₃ alkyl, C₁-C₃ alkoxy, and C₁-C₃ haloalkyl;
or a pharmaceutically acceptable salt thereof.
30. The compound of embodiment 28 or 29 or a pharmaceutically acceptable salt thereof, wherein R^{1B} is H.
31. The compound of any one of embodiments 28-30 or a pharmaceutically acceptable salt thereof, wherein R^{3B} is H.
32. The compound of any one of embodiments 28-31 or a pharmaceutically acceptable salt thereof, wherein Z^{2B} is CR^{2B}.
33. The compound of any one of embodiments 28-32 or a pharmaceutically acceptable salt thereof, wherein ring B is absent.
34. The compound of any one of embodiments 28-33 or a pharmaceutically acceptable salt thereof, wherein R^{7B} is H.
35. The compound of any one of embodiments 28-34 or a pharmaceutically acceptable salt thereof, wherein G^{B} is —NR^{4B}—(CR^{1B})₂₋₃—NR^{5B}R^{6B}.
36. The compound of any one of embodiments 28-34 or a pharmaceutically acceptable salt thereof, wherein G^{B} is a group of the formula

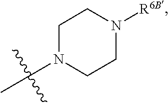

where R^{6B′} is selected from H, C₁-C₃ alkyl, and —C(O)—R^{10B}.
37. The compound of any one of embodiments 28-34 or a pharmaceutically acceptable salt thereof, wherein G^{B} is a group of the formula

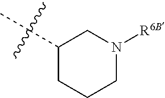

where R^{6B′} is selected from H, C₁-C₃ alkyl, and —C(O)—R^{10B}.
38. A compound of Formula (IV):

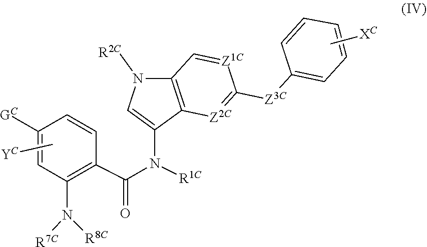

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Z^{1C} and Z^{2C} are independently selected from N and CH;
Z^{3C} is selected from O, CH₂, and NR^{3C};
G^{C} is a group of the formula —NR^{4C}—(CR^{2C})₂₋₃—NR^{5C}R^{6C} or G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C};
X^{C} represents one or two optional substituents independently selected from halo, C₁-C₃ alkyl, C₁-C₃ alkoxy, and C₁-C₃ haloalkyl;
Y^{C} represents one or two optional substituents independently selected from halo, C₁-C₃ alkyl, C₁-C₃ alkoxy, and C₁-C₃ haloalkyl;
R^{1C} is selected from H and C₁-C₃ alkyl;
R^{2C} is selected from H and C₁-C₃ alkyl;
R^{3C} is selected from H and C₁-C₃ alkyl;
R^{4C} is selected from H, C₁-C₃ alkyl, and —C(O)—R^{10C};
R^{5C} is selected from H and C₁-C₃ alkyl;
R^{6C} is selected from H, C₁-C₃ alkyl, and —C(O)—R^{10C};
R^{7C} is selected from H, C₁-C₄ alkyl optionally substituted with C₁-C₃ alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl;
R^{8C} is selected from H, C₁-C₃ alkyl and —C(O)—R^{10C};
each R^{10C} is independently selected from H, C₁-C₃ alkyl, C₁-C₃ alkoxy, and C₁-C₃ haloalkyl.
39. A compound of Formula (IV):

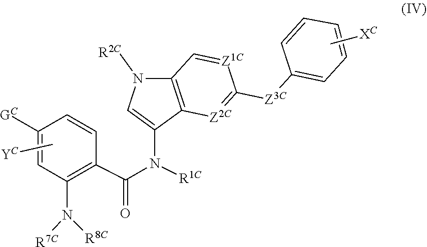

wherein:
Z^{1C} and Z^{2C} are independently selected from N and CH;
Z^{3C} is selected from O, CH₂, and NR^{3C};
G^{C} is a group of the formula —NR^{4C}—(CR₂)₂₋₃—NR^{5C}R^{6C} or G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C};
X^{C} represents one or two optional substituents selected from halo, C₁-C₃ alkyl, C₁-C₃ alkoxy, and C₁-C₃ haloalkyl;
Y^{C} represents one or two optional substituents selected from halo, C₁-C₃ alkyl, C₁-C₃ alkoxy, and C₁-C₃ haloalkyl;
R^{1C} is selected from H and C₁-C₃ alkyl;
R^{2C} is selected from H and C₁-C₃ alkyl;
R^{3C} is selected from H and C₁-C₃ alkyl;
R^{4C} is selected from H, C₁-C₃ alkyl, and —C(O)—R^{10C};
R^{5C} is selected from H and C₁-C₃ alkyl;
R^{6C} is selected from H, C₁-C₃ alkyl, and —C(O)—R^{10C};
R^{7C} is selected from H, C₁-C₄ alkyl optionally substituted with C₁-C₃ alkoxy, and a 5-6 membered heterocyclic group containing a heteroatom selected from N, O and S as a ring member, and optionally substituted with one or two groups selected from halo, C₁-C₃ alkyl, C₁-C₃alkoxy, and C₁-C₃ haloalkyl;
R^{8C} is selected from H, C₁-C₃ alkyl and —C(O)—R^{10C};
each R^{10C} is independently selected from H, C₁-C₃ alkyl, C₁-C₃ alkoxy, and C₁-C₃ haloalkyl;
or a pharmaceutically acceptable salt thereof.
40. The compound of embodiment 38 or 39 or a pharmaceutically acceptable salt thereof, wherein R^{1C} is H.
41. The compound of any one of embodiments 38-40 or a pharmaceutically acceptable salt thereof, wherein R^{2C} is H.
42. The compound of any one of embodiment 38-41 or a pharmaceutically acceptable salt thereof, wherein X^{C} represents one or two independently selected halo groups.
43. The compound of any one of embodiments 38-42 or a pharmaceutically acceptable salt thereof, wherein Y^{C} is absent.
44. The compound of any one of embodiments 38-43 or a pharmaceutically acceptable salt thereof, wherein Z^{1C} and Z^{2C} each represent CH.
45. The compound of any one of embodiments 38-43 or a pharmaceutically acceptable salt thereof, wherein Z^{1C} and Z^{2C} each represent N.
46. The compound of any one of embodiments 38-45 or a pharmaceutically acceptable salt thereof, wherein Z^{3C} is O.
47. The compound of any one of embodiments 38-45 or a pharmaceutically acceptable salt thereof, wherein Z^{3C} is NH.
48. The compound of any one of embodiments 38-45 or a pharmaceutically acceptable salt thereof, wherein Z^{3C} is CH₂.
49. The compound of any one of embodiments 38-48, or a pharmaceutically acceptable salt thereof, wherein R^{8C} is H or methyl.
50. The compound of any one of embodiments 38-49 or a pharmaceutically acceptable salt thereof, wherein R^{7C} is a tetrahydrofuran ring or a tetrahydropyran ring.
51. The compound of any one of embodiments 38-49 or a pharmaceutically acceptable salt thereof, wherein R^{7C} is C₂-C₄ alkyl substituted by methoxy.
52. The compound of any one of embodiments 38-51 or a pharmaceutically acceptable salt thereof, wherein G^{C} is a group of the formula

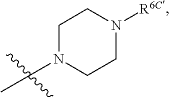

where R^{6C′} is selected from H, C₁-C₃ alkyl, and —C(O)—R^{10C}.
53. The compound of any one of embodiments 38-51 or a pharmaceutically acceptable salt thereof, wherein G^{C} is a group of the formula —NR^{4C}—(CR^{2C})₂₋₃—NR^{5C}R^{6C}.
54. The compound of embodiment 53 or a pharmaceutically acceptable salt thereof, wherein G^{C} is a group of the formula NR^{4C}—(CH₂)₂₋₃—NR^{5C}R^{6C}, wherein R^{4C}, R^{5C} and R^{6C} are each selected independently from methyl and ethyl.
55. A compound selected from the example compounds represented by the Compound ID numbers in Table 1.
56. A pharmaceutical composition comprising a compound of any of embodiments 1-55, or a pharmaceutically acceptable salt thereof, and at least one pharmaceutically acceptable excipient.
57. A pharmaceutical combination comprising a compound of any one of embodiments 1-55, or a pharmaceutically acceptable salt thereof, and an additional therapeutic agent.
58. A method to treat a cancer, which comprises administering to a subject in need of such treatment a compound of any one of embodiments 1-55, or a pharmaceutically acceptable salt thereof, or the pharmaceutical composition of embodiment 56, or the pharmaceutical embodiment of claim 57, wherein the cancer is selected from acute myeloid leukemia (AML), hepatocellular carcinoma (HCC), thyroid cancer, mast cell tumors (MCT), solid tumors with an NTRK gene fusion, leukemia, lymphoma, lung cancer including non-small cell lung cancer, colon and colorectal cancer, CNS cancer, melanoma, ovarian cancer, renal cancer, prostate cancer, breast cancer, liver cancer, sarcoma, epidermoid cancer, fibrosarcoma, cervical cancer, gastric carcinoma, skin cancer, head and neck cancers, and pancreatic cancer.
59. The method of embodiment 58, wherein the cancer is associated with a kinase selected from FLT3, EGFR, VEGFR, ALK, NTRK, RET, ROS/ROS1, DYRK1 and CK2a kinases.
60. The method of embodiment 58, wherein the cancer is AML, lung cancer, or colorectal cancer.
61. A compound according to any one of embodiments 1-55, or a pharmaceutically acceptable salt thereof, for use in therapy.
62. The compound or a pharmaceutically acceptable salt thereof according to embodiment 61, wherein therapy is for treatment of a cancer.
63. The method of embodiment 58 or the compound, or a pharmaceutically acceptable salt thereof, according to embodiment 62, wherein the cancer is associated with a kinase selected from FLT3, EGFR, VEGFR, ALK, NTRK, RET, ROS/ROS1, DYRK1 and CK2a kinases, and wherein the cancer is selected from AML, NSCLC, MCT (mast cell tumor), thyroid cancer and solid tumors with a NTRK gene fusion.
64. Use of a compound or a pharmaceutically acceptable salt thereof according to any one of embodiments 1-55 in the manufacture of a medicament.
65. The use of embodiment 64, wherein the medicament is for treating a cancer.
66. The use of embodiment 65, wherein the cancer is associated with a kinase selected from FLT3, ALK, EGFR, VEGFR, NTRK, RET, ROS/ROS1, DYRK1 and CK2a, and wherein the cancer is selected from AML, NSCLC, HCC, MCT, thyroid cancer and solid tumors with a NTRK gene fusion.

### Compounds

In an aspect, provided herein is a compound of Formula (I):

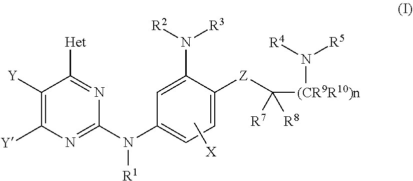

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹, wherein R¹¹ is selected from H, C₁-C₃ alkyl, C₁-C₃ alkoxy, and C₁-C₃ haloalkyl;
R⁴ is selected from H and C₁-C₃ alkyl;
R⁵ is selected from H, C₁-C₃ alkyl, C₁-C₃ haloalkyl, and —C(O)—R¹², wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³, wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl;
n is 1 to 4.

In an aspect, provided herein is a compound of Formula (I):

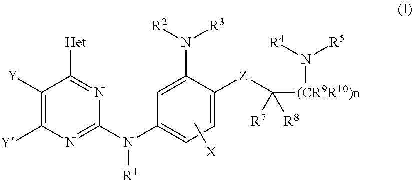

or an enantiomer, a mixture of enantiomers, a mixture of two or more diastereomers, a tautomer, a mixture of two or more tautomers, or an isotopic variant thereof; or a pharmaceutically acceptable salt thereof; wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl or
R⁴ is selected from H and C₁-C₃ alkyl;
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
   wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl;
n is 1 to 4.

In embodiments, Y is a group selected from H, halo, C₁-C₃ alkyl, C₁-C₃ alkoxy, C₁-C₃haloalkyl, and amino. In embodiments, Y is H. In embodiments, Y is halo. In embodiments, Y is C₁-C₃ alkyl. In embodiments, Y is C₁-C₃ alkoxy. In embodiments, Y is C₁-C₃ haloalkyl. In embodiments, Y is amino.

In embodiments, Y is a group selected from H, fluoro, chloro, bromo, iodo, methyl, ethyl, propyl, isopropyl, methoxy, ethoxy, propoxy, amino, —CF₃, —CHF₂, —CH₂F, —CCl₃, —CHCl₂, and —CH₂Cl. In embodiments, Y is a group selected from H, fluoro, chloro, methoxy, and —CF₃. In embodiments, Y is a group selected from fluoro, chloro, methoxy, and —CF₃.

In embodiments, Y is H. In embodiments, Y is fluoro. In embodiments, Y is chloro. In embodiments, Y is —CF₃. In embodiments, Y is methoxy.

In embodiments, Y′ is a group selected from H, halo, C₁-C₃ alkyl, C₁-C₃ alkoxy, C₁-C₃haloalkyl, and amino. In embodiments, Y′ is H. In embodiments, Y′ is halo. In embodiments, Y′ is C₁-C₃ alkyl. In embodiments, Y′ is C₁-C₃ alkoxy. In embodiments, Y′ is C₁-C₃ haloalkyl. In embodiments, Y′ is amino. In embodiments, Y′ is a group selected from H, fluoro, chloro, bromo, iodo, methyl, ethyl, propyl, isopropyl, methoxy, ethoxy, propoxy, amino, —CF₃, —CHF₂, —CH₂F, —CCl₃, —CHCl₂, and —CH₂Cl. In embodiments, Y′ is a group selected from H, methoxy and amino.

In embodiments, Y′ is H. In embodiments, Y′ is methoxy. In embodiments, Y′ is amino.

In embodiments, X represents one or two optional substituents independently selected from halo, C₁-C₃ alkyl, C₁-C₃ alkoxy, and C₁-C₃ haloalkyl. In embodiments, X represents two optional substituents independently selected from halo, C₁-C₃ alkyl, C₁-C₃ alkoxy, and C₁-C₃ haloalkyl. In embodiments, X is one optional substituent selected from halo, C₁-C₃ alkyl, C₁-C₃ alkoxy, and C₁-C₃ haloalkyl.

In embodiments, X is one optional substituent selected from fluoro, chloro, bromo, iodo, methyl, ethyl, propyl, isopropyl, methoxy, ethoxy, propoxy, —CF₃, —CHF₂, —CH₂F, —CCl₃, —CHCl₂, and —CH₂Cl.

In embodiments, X is halo. In embodiments, X is C₁-C₃ alkyl. In embodiments, X is C₁-C₃ alkoxy. In embodiments, X is C₁-C₃ haloalkyl. In embodiments, X is fluoro. In embodiments, X is chloro. In embodiments, X is methyl. In embodiments, X is ethyl. In embodiments, X is methoxy. In embodiments, X is ethoxy. In embodiments, X is —CF₃. In embodiments, X is —CCl₃.

In embodiments, R¹ is selected from H and C₁-C₃ alkyl. In embodiments, R¹ is H. In embodiments, R¹ is C₁-C₃ alkyl. In embodiments, R¹ is methyl. In embodiments, R¹ is ethyl. In embodiments, R¹ is propyl. In embodiments, R¹ is isopropyl.

In embodiments, R² is selected from H and C₁-C₃ alkyl. In embodiments, R² is H. In embodiments, R² is C₁-C₃ alkyl. In embodiments, R² is methyl. In embodiments, R² is ethyl. In embodiments, R² is propyl. In embodiments, R² is isopropyl.

In embodiments, R³ is selected from H, C₁-C₃ alkyl, C₁-C₃ haloalkyl, and —C(O)—R¹¹. In embodiments, R³ is selected from H, methyl, ethyl, propyl, isopropyl, C₁-C₃ haloalkyl, —C(O)Me, and —C(O)Et. In embodiments, R³ is H. In embodiments, R³ is methyl. In embodiments, R³ is ethyl. In embodiments, R³ is propyl. In embodiments, R³ is isopropyl. In embodiments, R³ is C₁-C₃ haloalkyl. In embodiments, R³ is —C(O)Me. In embodiments, R³ is —C(O)Et.

In embodiments, R³ is —CF₃. In embodiments, R³ is —CHF₂. In embodiments, R³ is —CH₂F. In embodiments, R³ is —CH₂CF₃. In embodiments, R³ is —CH₂CH₂CF₃.

In embodiments, R⁴ is selected from H and C₁-C₃ alkyl. In embodiments, R⁴ is H. In embodiments, R⁴ is C₁-C₃ alkyl. In embodiments, R⁴ is methyl. In embodiments, R⁴ is ethyl. In embodiments, R⁴ is propyl. In embodiments, R⁴ is isopropyl.

In embodiments, R⁵ is selected from H, C₁-C₃ alkyl, C₁-C₃ haloalkyl, and —C(O)—R¹². In embodiments, R⁵ is selected from H, methyl, ethyl, propyl, isopropyl, C₁-C₃ haloalkyl, —C(O)Me, and —C(O)Et. In embodiments, R⁵ is H. In embodiments, R⁵ is methyl. In embodiments, R⁵ is ethyl. In embodiments, R⁵ is propyl. In embodiments, R⁵ is isopropyl. In embodiments, R⁵ is C₁-C₃ haloalkyl. In embodiments, R⁵ is —C(O)Me. In embodiments, R⁵ is —C(O)Et.

In embodiments, R⁶ is selected from H, C₁-C₃ alkyl, and —C(O)—R¹³. In embodiments, R⁶ is selected from H, methyl, ethyl, propyl, isopropyl, —C(O)Me, and —C(O)Et. In embodiments, R⁶ is H. In embodiments, R⁶ is methyl. In embodiments, R⁶ is ethyl. In embodiments, R⁶ is propyl. In embodiments, R⁶ is isopropyl. In embodiments, R⁶ is —C(O)Me. In embodiments, R⁶ is —C(O)Et.

In embodiments, R¹¹ is selected from H, C₁-C₃ alkyl, C₁-C₃ alkoxy, and C₁-C₃ haloalkyl. In embodiments, R¹¹ is H. In embodiments, R¹¹ is C₁-C₃ alkyl. In embodiments, R¹¹ is selected from methyl, ethyl, propyl, and isopropyl. In embodiments, R¹¹ is methyl. In embodiments, R¹¹ is ethyl. In embodiments, R¹¹ is propyl. In embodiments, R¹¹ is isopropyl.

In embodiments, R¹² is selected from H, C₁-C₃ alkyl, C₁-C₃ alkoxy, and C₁-C₃ haloalkyl. In embodiments, R¹² is H. In embodiments, R¹² is C₁-C₃ alkyl. In embodiments, R¹² is selected from methyl, ethyl, propyl, and isopropyl. In embodiments, R¹² is methyl. In embodiments, R¹² is ethyl. In embodiments, R¹² is propyl. In embodiments, R¹² is isopropyl.

In embodiments, Z is selected from —O—, —NR⁶—, —C(═O)—, —SO₂—, —C(═O)NR⁶—, —NR⁶C(═O)—, —SO₂NR⁶—, and —(CH₂)₁₋₂—. In embodiments, Z is selected from —O—, —NH—, —NMe-, —NEt-, —N(CH₂)₂CH₃—, —NC(═O)CH₃—, —NC(═O)CH₂CH₃—, —C(O)—, —SO₂—, —NHC(═O)—, -(Me)NC(═O)—, -(Et)NC(═O)—, —SO₂NH—, —SO₂NMe-, —(CH₂)—, and —(CH₂)₂—. In embodiments, Z is selected from —O—, —NH—, —NMe-, —NEt-, —NHC(═O)—, -(Me)NC(═O)—, -(Et)NC(═O)—, —(CH₂)—, and —(CH₂)₂—.

In embodiments, Z is —O—. In embodiments, Z is —NR⁶—. In embodiments, Z is —CO—. In embodiments, Z is —SO₂—. In embodiments, Z is —C(═O)NR⁶—. In embodiments, Z is —NR⁶C(═O)—. In embodiments, Z is —SO₂NR⁶—. In embodiments, Z is —(CH₂)—. In embodiments, Z is —(CH₂CH₂)—. In embodiments, Z is —NH—. In embodiments, Z is —NMe-. In embodiments, Z is —NEt-. In embodiments, Z is —NHC(═O)—. In embodiments, Z is -(Me)NC(═O)—. In embodiments, Z is -(Et)NC(═O)—. In embodiments, Z is —C(═O)NCH₃—.

In embodiments, R¹³ is selected from H, C₁-C₃ alkyl, C₁-C₃ alkoxy, and C₁-C₃ haloalkyl. In embodiments, R¹³ is H. In embodiments, R¹³ is C₁-C₃ alkyl. In embodiments, R¹³ is selected from methyl, ethyl, propyl, and isopropyl. In embodiments, R¹³ is methyl. In embodiments, R¹³ is ethyl. In embodiments, R¹³ is propyl. In embodiments, R¹³ is isopropyl.

In embodiments, R⁷ and R⁸ are each independently selected from H and C₁-C₃ alkyl; or R⁷ and R⁸ taken together can represent oxo (═O). In embodiments, R⁷ and R⁸ are each H. In embodiments, R⁷ and R⁸ taken together can represent oxo (═O).

In embodiments, R⁹ and R¹⁰ are each independently selected from H and C₁-C₃ alkyl.

In embodiments, R⁹ and R¹⁰ are each independently selected from H, methyl, ethyl, propyl, and isopropyl. In embodiments, R⁹ and R¹⁰ are each H.

In embodiments, R⁷, R⁸, R⁹ and R¹⁰ are each H.

In embodiments, n is 1 to 4. In embodiments, n is 1. In embodiments, n is 2. In embodiments, n is 3. In embodiments, n is 4.

In embodiments, Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl. In embodiments, Het represents a 5-9 atom heteroaromatic bicyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl. In embodiments, Het represents a 5-9 atom heteroaromatic bicyclic group comprising at least one nitrogen atom as a ring member, and Het is optionally substituted with one to three groups independently selected from halo, C₁-C₃ alkoxy, C₁-C₃ haloalkyl, and C₁-C₃ alkyl.

In embodiments, Het is an indole. In embodiments, Het is an optionally substituted indole. In embodiments, Het is an indole optionally substituted with one to three groups independently selected from halo, C₁-C₃ alkoxy, C₁-C₃ haloalkyl, and C₁-C₃ alkyl. In embodiments, Het is an indole optionally substituted with one group selected from halo, C₁-C₃ alkoxy, C₁-C₃ haloalkyl, and C₁-C₃ alkyl. In embodiments, Het is an indole optionally substituted with two groups independently selected from halo, C₁-C₃ alkoxy, C₁-C₃ haloalkyl, and C₁-C₃ alkyl. In embodiments, Het is an indole optionally substituted with three groups independently selected from halo, C₁-C₃ alkoxy, C₁-C₃ haloalkyl, and C₁-C₃ alkyl.

In embodiments, Het is an indole optionally substituted with one group selected from fluoro, methyl, methoxy, and ethyl. In embodiments, Het is an indole optionally substituted with two groups independently selected from fluoro, methyl, methoxy, and ethyl. In embodiments, Het is an indole optionally substituted with three groups independently selected from fluoro, methyl, methoxy, and ethyl.

In embodiments, Het represents a 5-9 atom heteroaromatic monocyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl. In embodiments, Het represents a 5 atom heteroaromatic monocyclic group comprising at least one heteroatom selected from N, O and S as a ring member, and Het is optionally substituted with one to three groups independently selected from halo, C₁-C₃ alkoxy, C₁-C₃ haloalkyl, and C₁-C₃ alkyl. In embodiments, Het represents a 5 atom heteroaromatic monocyclic group comprising at least one nitrogen atom as a ring member, and Het is optionally substituted with one to three groups independently selected from halo, C₁-C₃ alkoxy, C₁-C₃ haloalkyl, and C₁-C₃ alkyl. In embodiments, Het represents a 5 atom heteroaromatic monocyclic group comprising two nitrogen atoms as ring members, and Het is optionally substituted with one to three groups independently selected from halo, C₁-C₃ alkoxy, C₁-C₃ haloalkyl, and C₁-C₃ alkyl.

In embodiments, Het is a pyrazole. In embodiments, Het is a pyrazole optionally substituted with one to three groups independently selected from halo, C₁-C₃ alkoxy, C₁-C₃ haloalkyl, and C₁-C₃ alkyl. In embodiments, Het is a pyrazole optionally substituted with one group selected from halo, C₁-C₃ alkoxy, C₁-C₃ haloalkyl, and C₁-C₃ alkyl. In embodiments, Het is a pyrazole optionally substituted with two groups independently selected from halo, C₁-C₃ alkoxy, C₁-C₃ haloalkyl, and C₁-C₃ alkyl. In embodiments, Het is a pyrazole optionally substituted with three groups independently selected from halo, C₁-C₃ alkoxy, C₁-C₃ haloalkyl, and C₁-C₃ alkyl.

In embodiments, Het is a pyrazole optionally substituted with one group selected from fluoro, methyl, methoxy, and ethyl. In embodiments, Het is a pyrazole optionally substituted with two groups independently selected from fluoro, methyl, methoxy, and ethyl. In embodiments, Het is a pyrazole optionally substituted with three groups independently selected from fluoro, methyl, methoxy, and ethyl.

In embodiments, Het is a pyrazole optionally substituted with methyl and ethyl. In embodiments, Het is a pyrazole optionally substituted with two methyl groups.

In embodiments, Het is an imidazole. In embodiments, Het is an imidazole optionally substituted with one to three groups independently selected from halo, C₁-C₃ alkoxy, C₁-C₃ haloalkyl, and C₁-C₃ alkyl. In embodiments, Het is an imidazole optionally substituted with one group selected from halo, C₁-C₃ alkoxy, C₁-C₃ haloalkyl, and C₁-C₃ alkyl. In embodiments, Het is an imidazole optionally substituted with two groups independently selected from halo, C₁-C₃ alkoxy, C₁-C₃ haloalkyl, and C₁-C₃ alkyl. In embodiments, Het is an imidazole optionally substituted with three groups independently selected from halo, C₁-C₃ alkoxy, C₁-C₃ haloalkyl, and C₁-C₃ alkyl.

In embodiments, Het is an imidazole optionally substituted with one group selected from fluoro, methyl, methoxy, isopropyl and ethyl. In embodiments, Het is an imidazole optionally substituted with two groups independently selected from fluoro, methyl, methoxy, isopropyl, and ethyl. In embodiments, Het is an imidazole optionally substituted with three groups independently selected from fluoro, methyl, methoxy, isopropyl, and ethyl.

In embodiments, Het is an imidazole optionally substituted with methyl and isopropyl.

In embodiments, Het is selected from:

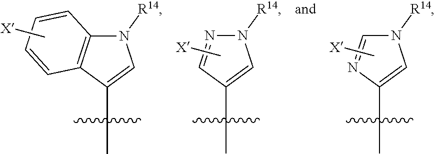

wherein each X′ represents up to two optional substituents independently selected from halo, C₁-C₃ alkyl, and C₁-C₃ alkoxy; each R¹⁴ represents H; C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy; or —C(═O)—R* where R* is H, C₁₋₃ haloalkyl, or C₁₋₄ alkyl optionally substituted with OH, CN, or C₁₋₃ alkoxy.

In embodiments, Het is

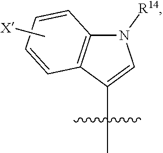

wherein X′ represents one optional substituent selected from halo, C₁-C₃ alkyl, and C₁-C₃ alkoxy; and R¹⁴ is H, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy; or —C(═O)—R* where R* is H, C₁₋₃ haloalkyl, or C₁₋₄ alkyl optionally substituted with OH, CN, or C₁₋₃ alkoxy.

In embodiments, Het is

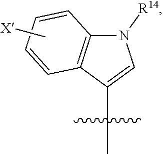

wherein X′ represents one optional substituent selected from fluoro, chloro, bromo, iodo, methyl, ethyl, propyl, isopropyl, methoxy, ethoxy, and propoxy. In embodiments, Het is

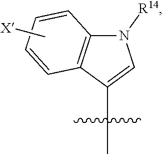

wherein X′ represents one optional substituent selected from fluoro, methyl, ethyl, and methoxy. In embodiments, Het is

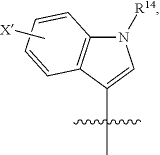

wherein X′ is fluoro. In embodiments, Het is

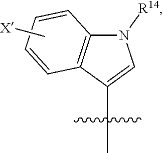

wherein X′ is methyl. In embodiments, Het is

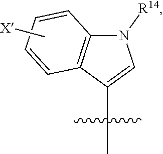

wherein X′ ethyl. In embodiments, Het is

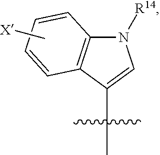

wherein X′ is methoxy.

In embodiments, Het is

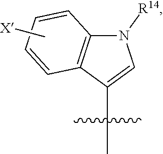

wherein X′ represents two optional substituents independently selected from halo, C₁-C₃ alkyl, and C₁-C₃ alkoxy; and R¹⁴ is H, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy; or —C(═O)—R* where R* is H, C₁₋₃ haloalkyl, or C₁₋₄ alkyl optionally substituted with OH, CN, or C₁₋₃ alkoxy.

In embodiments, Het is

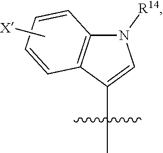

wherein X′ represents two optional substituents independently selected from fluoro, chloro, bromo, iodo, methyl, ethyl, propyl, isopropyl, methoxy, ethoxy, and propoxy. In embodiments, Het is

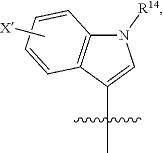

wherein X′ represents two optional substituents independently selected from fluoro, methyl, ethyl, and methoxy. In embodiments, X′ is absent. In embodiments, Het is

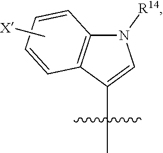

wherein R¹⁴ is selected from H, methyl, ethyl, isopropyl, cyclopropyl and —C(═O)—R*. In embodiments, R¹⁴ is H, methyl, or ethyl. In embodiments, R¹⁴ is H. In embodiments, R¹⁴ is methyl. In embodiments, R¹⁴ is ethyl.

In embodiments, R* is H, C₁₋₃ haloalkyl, or C₁₋₄ alkyl optionally substituted with OH, CN, or C₁₋₃ alkoxy. In embodiments, R* is H, methyl, ethyl, propyl, or isopropyl. In embodiments, R* is H. In embodiments, R* is methyl. In embodiments, R* is ethyl. In embodiments, R* is propyl. In embodiments, R* is isopropyl.

In embodiments, provided herein is a compound of Formula (IA):

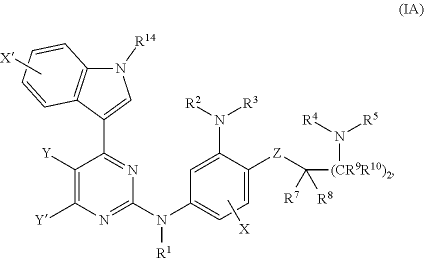

or an enantiomer, a mixture of enantiomers, a mixture of two or more diastereomers, a tautomer, a mixture of two or more tautomers, or an isotopic variant thereof; or a pharmaceutically acceptable salt thereof; wherein R¹⁴ is selected from H, methyl, ethyl, isopropyl, cyclopropyl and —C(═O)—R* where R* is H, C₁₋₃ haloalkyl, or C₁₋₄ alkyl optionally substituted with OH, CN, or C₁₋₃ alkoxy; and wherein R¹, R², R³, R⁴, R⁵, R⁷, R⁸, R⁹, R¹⁰, X, X′, Y, Y′, and Z are each as defined herein.

In embodiments, provided herein is a compound of Formula (IB):

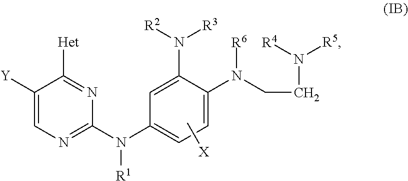

or an enantiomer, a mixture of enantiomers, a mixture of two or more diastereomers, a tautomer, a mixture of two or more tautomers, or an isotopic variant thereof; or a pharmaceutically acceptable salt thereof; wherein R⁴, R⁵, and R⁶ are each independently selected from methyl and ethyl; and wherein R¹, R², R³, X, Y, and Het are each as defined herein.

In an aspect, provided herein is a compound of Formula (IC):

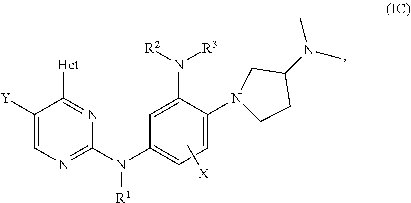

or an enantiomer, a mixture of enantiomers, a mixture of two or more diastereomers, a tautomer, a mixture of two or more tautomers, or an isotopic variant thereof; or a pharmaceutically acceptable salt thereof; wherein
X represents one or two optional substituents independently selected from halo, C₁-C₃ alkyl, C₁-C₃ alkoxy, and C₁-C₃ haloalkyl;
Y is selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl; and
R³ is selected from H, C₁-C₃ alkyl, C₁-C₃ haloalkyl, and —C(O)—R¹¹,
   wherein R¹¹ is selected from H, C₁-C₃ alkyl, C₁-C₃ alkoxy, and C₁-C₃ haloalkyl.

In an aspect, provided herein is a compound of Formula (ID):

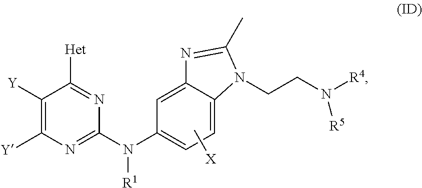

or an enantiomer, a mixture of enantiomers, a mixture of two or more diastereomers, a tautomer, a mixture of two or more tautomers, or an isotopic variant thereof; or a pharmaceutically acceptable salt thereof; wherein
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R⁴ is selected from H and C₁-C₃ alkyl; and
R⁵ is selected from H, C₁-C₃ alkyl, C₁-C₃ haloalkyl, and —C(O)—R¹²,
   wherein R¹² is selected from H, C₁-C₃ alkyl, C₁-C₃ alkoxy, and C₁-C₃ haloalkyl.

In an aspect, provided herein is a compound of Formula (IE):

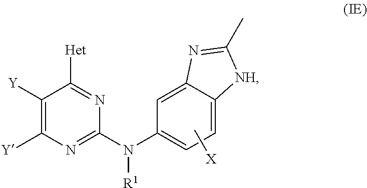

or an enantiomer, a mixture of enantiomers, a mixture of two or more diastereomers, a tautomer, a mixture of two or more tautomers, or an isotopic variant thereof; or a pharmaceutically acceptable salt thereof; wherein
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole; and
R¹ is selected from H and C₁-C₃ alkyl.

In an aspect, provided herein is a compound of Formula (II):

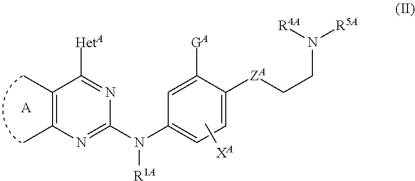

or an enantiomer, a mixture of enantiomers, a mixture of two or more diastereomers, a tautomer, a mixture of two or more tautomers, or an isotopic variant thereof; or a pharmaceutically acceptable salt thereof; wherein:
   Ring A is an optional 5-6 membered heterocyclic ring fused to the pyrimidine in Formula (II), comprising one or two heteroatoms selected from N and O as ring members,
   wherein Ring A can be aromatic or non-aromatic and is optionally substituted with one or two groups independently selected from halo, C₁-C₃ alkoxy, C₁-C₃ haloalkyl, and C₁-C₃ alkyl;
   Alternatively, Ring A is absent, and pyrimidine is optionally substituted with one or two groups independently selected from halo, C₁-C₃ alkoxy, C₁-C₃ haloalkyl, and C₁-C₃ alkyl;
   Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl;
   X^{A} represents one or two optional substituents independently selected from halo, C₁-C₃alkyl, C₁-C₃ alkoxy, and C₁-C₃ haloalkyl;
   R^{1A} is selected from H and C₁-C₃ alkyl;
   G^{A} is selected from —NR^{2A}R^{3A}, SO₂R^{7A}, halo, and C₁-C₃ haloalkyl;
      wherein R^{2A} is selected from H and C₁-C₃ alkyl, and R^{3A} is selected from H, C₁-C₃ alkyl, C₂-C₄ alkenyl, C₁-C₃ haloalkyl, —SO₂R^{7A}, and —C(O)—R^{11A},
      wherein R^{11A} is selected from H, C₁-C₃ alkyl, C₂-C₄ alkenyl, C₁-C₃ alkoxy, and C₁-C₃ haloalkyl;
   Z^{A} is selected from O, NR^{6A}, C(═O), SO₂, C(═O)NR^{6A}, SO₂NR^{6A}, and (CH₂)₁₋₂;
   R^{4A} is selected from H and C₁-C₃ alkyl;
   R^{5A} is selected from H, C₁-C₃ alkyl, C₁-C₃ haloalkyl, —C(O)—R^{12A}, and —SO₂R^{7A},
      wherein R^{12A} is selected from H, C₁-C₃ alkyl, C₁-C₃ alkoxy, and C₁-C₃ haloalkyl;
   R^{6A} is selected from H and C₁-C₃ alkyl; and
   each R^{7A} is independently C₁-C₃ alkyl.

In embodiments, R^{1A} is selected from H and C₁-C₃ alkyl. In embodiments, R^{1A} is H. In embodiments, R^{1A} is C₁-C₃ alkyl. In embodiments, R^{1A} is methyl. In embodiments, R^{1A} is ethyl. In embodiments, R^{1A} is propyl. In embodiments, R^{1A} is isopropyl.

In embodiments, R^{2A} is selected from H and C₁-C₃ alkyl. In embodiments, R^{2A} is H. In embodiments, R^{2A} is C₁-C₃ alkyl. In embodiments, R^{2A} is methyl. In embodiments, R^{2A} is ethyl. In embodiments, R^{2A} ispropyl. In embodiments, R^{2A} is isopropyl.

In embodiments, R^{3A} is selected from H, C₁-C₃ alkyl, C₂-C₄ alkenyl, C₁-C₃ haloalkyl, —SO₂R^{7A}, and —C(O)—R^{11A}. In embodiments, R^{3A} is selected from H, methyl, ethyl, propyl, isopropyl, propenyl, butenyl, —SO₂Me, —C(O)Me, —C(O)Et, —CH₂CF₃, —C(O)CH═CH₂, —C(O)CH(CH₃)₂, and —CH₂CH₂F. In embodiments, R^{3A} is selected from H, methyl, ethyl, —SO₂Me, —C(O)Me, —C(O)Et, —CH₂CF₃, —C(O)CH═CH₂, —C(O)CH(CH₃)₂, and —CH₂CH₂F.

In embodiments, R^{3A} is H. In embodiments, R^{3A} is methyl. In embodiments, R^{3A} is ethyl. In embodiments, R^{3A} is —SO₂Me. In embodiments, R^{3A} is —C(O)Me. In embodiments, R^{3A} is —C(O)Et. In embodiments, R^{3A} is —CH₂CF₃. In embodiments, R^{3A} is —C(O)CH═CH₂. In embodiments, R^{3A} is —C(O)CH(CH₃)₂. In embodiments, R^{3A} is —CH₂CH₂F.

In embodiments, G^{A} is selected from —NR^{2A}R^{3A}, —SO₂R^{7A}, halo, and C₁-C₃ haloalkyl. In embodiments, G^{A} is —NR^{2A}R^{3A}. In embodiments, G^{A} is —SO₂R^{7A}. In embodiments, G^{A} is halo. In embodiments, G^{A} is C₁-C₃ haloalkyl. In embodiments, G^{A} is selected from —NH₂, —NHMe, —NHEt, —N(CH₃)₂, —NHC(O)Me, —NHC(O)Et, —NHCH₂CF₃, —NHSO₂Me, —SO₂Me, —F, —Cl, —Br, —I, —NHC(O)CH═CH₂, —NHC(O)CH(CH₃)₂, —NHCH₂CH₂F, and C₁-C₃ haloalkyl. In embodiments, G^{A} is selected from —NH₂, —NHMe, —NHEt, —N(Me)₂, —NHC(O)Me, —NHC(O)Et, —NHCH₂CF₃, —NHSO₂Me, —NHC(O)CH═CH₂, —NHC(O)CH(CH₃)₂, —NHCH₂CH₂F, —SO₂Me, and —F.

In embodiments, G^{A} is —NH₂. In embodiments, G^{A} is —NHMe. In embodiments, G^{A} is —NHEt. In embodiments, G^{A} is —N(Me)₂. In embodiments, G^{A} is —NHC(O)Me. In embodiments, G^{A} is —NHC(O)Et. In embodiments, G^{A} is —NHCH₂CF₃. In embodiments, G^{A} is —NHSO₂Me. In embodiments, G^{A} is —NHC(O)CH═CH₂. In embodiments, G^{A} is —NHC(O)CH(CH₃)₂. In embodiments, G^{A} is —NHCH₂CH₂F. In embodiments, G^{A} is —SO₂Me. In embodiments, G^{A} is —F.

In embodiments, R^{4A} is selected from H and C₁-C₃ alkyl. In embodiments, R^{4A} is H. In embodiments, R^{4A} is C₁-C₃ alkyl. In embodiments, R^{4A} is methyl. In embodiments, R^{4A} is ethyl. In embodiments, R^{4A} is propyl. In embodiments, R^{4A} is isopropyl.

In embodiments, R^{5A} is selected from H, C₁-C₃ alkyl, C₁-C₃ haloalkyl, —C(O)—R^{12A}, and —SO₂R^{7A}. In embodiments, R^{5A} is selected from H, methyl, ethyl, propyl, isopropyl, C₁-C₃ haloalkyl, —SO₂Me, —C(O)Me, and —C(O)Et. In embodiments, R^{5A} is H. In embodiments, R^{5A} is methyl. In embodiments, R^{5A} is ethyl. In embodiments, R^{5A} is propyl. In embodiments, R^{5A} is isopropyl. In embodiments, R^{5A} is C₁-C₃ haloalkyl. In embodiments, R^{5A} is —SO₂Me. In embodiments, R^{5A} is —C(O)Me. In embodiments, R^{5A} is —C(O)Et.

In embodiments, R^{6A} is selected from H and C₁-C₃ alkyl. In embodiments, R^{6A} is H. In embodiments, R^{6A} is C₁-C₃ alkyl. In embodiments, R^{6A} is methyl. In embodiments, R^{6A} is ethyl. In embodiments, R^{6A} is propyl. In embodiments, R^{6A} is isopropyl.

In embodiments, R^{7A} is C₁-C₃ alkyl. In embodiments, R^{6A} is methyl. In embodiments, R^{6A} is ethyl. In embodiments, R^{6A} is propyl. In embodiments, R^{6A} is isopropyl.

In embodiments, R^{11A} is selected from H, C₁-C₃ alkyl, C₂-C₄ alkenyl, C₁-C₃ alkoxy, and C₁-C₃ haloalkyl. In embodiments, R^{11A} is selected from C₁-C₃ alkyl, and C₂-C₄ alkenyl. In embodiments, R^{11A} is C₁-C₃ alkyl. In embodiments, R^{11A} is C₂-C₄ alkenyl. In embodiments, R^{11A} is selected from methyl, ethyl, propyl, isopropyl, and ethenyl. In embodiments, R^{11A} is methyl. In embodiments, R^{11A} is ethyl. In embodiments, R^{11A} is propyl. In embodiments, R^{11A} is isopropyl. In embodiments, R^{11A} is ethenyl.

In embodiments, R^{12A} is selected from H, C₁-C₃ alkyl, C₁-C₃ alkoxy, and C₁-C₃ haloalkyl. In embodiments, R^{12A} is H. In embodiments, R^{12A} is C₁-C₃ alkyl. In embodiments, R^{12A} is selected from methyl, ethyl, propyl, and isopropyl. In embodiments, R^{12A} is methyl. In embodiments, R^{12A} is ethyl. In embodiments, R^{12A} is propyl. In embodiments, R^{12A} is isopropyl.

In embodiments, Z^{A} is selected from —O—, —NR^{6A}—, —C(═O)—, —SO₂—, —C(═O)NR^{6A}—, —SO₂NR^{6A}—, and —(CH₂)₁₋₂—. In embodiments, Z^{A} is selected from —O—, —NH—, —NMe-, —NEt-, —C(O)—, —SO₂—, —C(═O)NH—, —C(═O)NMe-, —C(═O)NEt-, —SO₂NH—, —SO₂NMe-, —(CH₂)—, and —(CH₂)₂—. In embodiments, Z^{A} is selected from —O—, —NH—, —NMe-, —NEt-, and —C(O)—.

In embodiments, Z^{A} is —NR^{6A}—. In embodiments, Z^{A} is —C(═O)NR^{6A}—. In embodiments, Z^{A} is —SO₂NR^{6A}—. In embodiments, Z^{A} is —(CH₂)—. In embodiments, Z^{A} is —(CH₂)₂—. In embodiments, Z^{A} is —O—. In embodiments, Z^{A} is —NH—. In embodiments, Z^{A} is —NMe-. In embodiments, Z^{A} is —NEt-. In embodiments, Z^{A} is —C(O)—.

In embodiments, X^{A} is one or two optional substituents independently selected from halo, C₁-C₃ alkyl, C₁-C₃ alkoxy, and C₁-C₃ haloalkyl. In embodiments, X^{A} is one optional substituent selected from halo, C₁-C₃ alkyl, C₁-C₃ alkoxy, and C₁-C₃ haloalkyl. In embodiments, X^{A} is two optional substituents independently selected from halo, C₁-C₃ alkyl, C₁-C₃alkoxy, and C₁-C₃ haloalkyl.

In embodiments, X^{A} is one optional substituent which is halo. In embodiments, X^{A} is one optional substituent which is chloro. In embodiments, X^{A} is one optional substituent which is bromo. In embodiments, X^{A} is one optional substituent which is iodo. In embodiments, X^{A} is one optional substituent which is fluoro.

In embodiments, X^{A} is one optional substituent which is C₁-C₃ alkyl. In embodiments, X^{A} is one optional substituent which is C₁-C₃ alkoxy. In embodiments, X^{A} is one optional substituent which is C₁-C₃ haloalkyl.

In embodiments, X^{A} is one optional substituent selected from fluoro, methyl, ethyl, or propyl.

In embodiments, X^{A} is two optional substituents independently selected from chloro, bromo, iodo and fluoro.

In embodiments, X^{A} is two optional substituents independently selected from chloro, bromo, iodo, fluoro, methyl, ethyl, propyl, methoxy, ethoxy, propoxy, and C₁-C₃ haloalkyl.

In embodiments, X^{A} is one optional substituent selected from fluoro, methyl, and ethyl.

In embodiments, X^{A} is absent. In embodiments, X^{A} is fluoro.

In embodiments, Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl. In embodiments, Het^{A} represents a 5-9 atom heteroaromatic bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl. In embodiments, Het^{A} represents a 5-9 atom heteroaromatic bicyclic group comprising at least one nitrogen atom as a ring member, and Het^{A} is optionally substituted with one to three groups independently selected from halo, C₁-C₃alkoxy, C₁-C₃ haloalkyl, and C₁-C₃ alkyl.

In embodiments, Het^{A} is an indole. In embodiments, Het^{A} is an optionally substituted indole. In embodiments, Het^{A} is an indole optionally substituted with one to three groups independently selected from halo, C₁-C₃ alkoxy, C₁-C₃ haloalkyl, and C₁-C₃ alkyl. In embodiments, Het^{A} is an indole optionally substituted with one group selected from halo, C₁-C₃ alkoxy, C₁-C₃ haloalkyl, and C₁-C₃ alkyl. In embodiments, Het^{A} is an indole optionally substituted with two groups independently selected from halo, C₁-C₃ alkoxy, C₁-C₃ haloalkyl, and C₁-C₃ alkyl. In embodiments, Het^{A} is an indole optionally substituted with three groups independently selected from halo, C₁-C₃ alkoxy, C₁-C₃ haloalkyl, and C₁-C₃ alkyl.

In embodiments, Het^{A} is an indole optionally substituted with one group selected from fluoro, methyl, methoxy, and ethyl. In embodiments, Het^{A} is an indole optionally substituted with two groups independently selected from fluoro, methyl, methoxy, and ethyl. In embodiments, Het^{A} is an indole optionally substituted with three groups independently selected from fluoro, methyl, methoxy, and ethyl.

In embodiments, Het^{A} represents a 5-9 atom heteroaromatic monocyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl. In embodiments, Het^{A} represents a 5 atom heteroaromatic monocyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl. In embodiments, Het^{A} represents a 5 atom heteroaromatic monocyclic group comprising at least one nitrogen atom as a ring member, and Het^{A} is optionally substituted with one to three groups independently selected from halo, C₁-C₃ alkoxy, C₁-C₃ haloalkyl, and C₁-C₃ alkyl. In embodiments, Het^{A} represents a 5 atom heteroaromatic monocyclic group comprising at two nitrogen atoms as ring members, and Het^{A} is optionally substituted with one to three groups independently selected from halo, C₁-C₃ alkoxy, C₁-C₃ haloalkyl, and C₁-C₃ alkyl.

In embodiments, Het^{A} is a pyrazole. In embodiments, Het^{A} is a pyrazole optionally substituted with one to three groups independently selected from halo, C₁-C₃ alkoxy, C₁-C₃ haloalkyl, and C₁-C₃ alkyl. In embodiments, Het^{A} is a pyrazole optionally substituted with one group selected from halo, C₁-C₃ alkoxy, C₁-C₃ haloalkyl, and C₁-C₃ alkyl. In embodiments, Het^{A} is a pyrazole optionally substituted with two groups independently selected from halo, C₁-C₃ alkoxy, C₁-C₃ haloalkyl, and C₁-C₃ alkyl. In embodiments, Het^{A} is a pyrazole optionally substituted with three groups independently selected from halo, C₁-C₃ alkoxy, C₁-C₃ haloalkyl, and C₁-C₃ alkyl.

In embodiments, Het^{A} is a pyrazole optionally substituted with one group selected from fluoro, methyl, methoxy, and ethyl. In embodiments, Het^{A} is a pyrazole optionally substituted with two groups independently selected from fluoro, methyl, methoxy, and ethyl. In embodiments, Het^{A} is a pyrazole optionally substituted with three groups independently selected from fluoro, methyl, methoxy, and ethyl.

In embodiments, Het^{A} is a pyrazole optionally substituted with two groups independently selected from methyl and ethyl. In embodiments, Het^{A} is a pyrazole optionally substituted with two methyl groups.

In embodiments, Ring A is a 5-6 membered heterocyclic ring fused to the pyrimidine in Formula (II), comprising one or two heteroatoms selected from N and O as ring members, optionally substituted with one or two groups independently selected from halo, C₁-C₃ alkoxy, C₁-C₃ haloalkyl, and C₁-C₃ alkyl. In embodiments, Ring A is an aromatic 5-6 membered heterocyclic ring fused to the pyrimidine in Formula (II), comprising one or two heteroatoms selected from N and O as ring members, optionally substituted with one or two groups independently selected from halo, C₁-C₃ alkoxy, C₁-C₃ haloalkyl, and C₁-C₃ alkyl. In embodiments, Ring A is a non-aromatic 5-6 membered heterocyclic ring fused to the pyrimidine in Formula (II), comprising one or two heteroatoms selected from N and O as ring members, optionally substituted with one or two groups independently selected from halo, C₁-C₃ alkoxy, C₁-C₃ haloalkyl, and C₁-C₃ alkyl.

In embodiments, Ring A is a 5-6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃alkyl. In embodiments, Ring A is an aromatic 5-6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl. In embodiments, Ring A is a non-aromatic 5-6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl.

In embodiments, Ring A is a 5 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃alkyl. In embodiments, Ring A is an aromatic 5 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl. In embodiments, Ring A is a non-aromatic 5 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl.

In embodiments, Ring A is a 6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃alkyl. In embodiments, Ring A is an aromatic 6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl. In embodiments, Ring A is a non-aromatic 6 membered heterocyclic ring fused to the pyrimidine in Formula (II), comprising one or two heteroatoms selected from N and O as ring members, optionally substituted with one group selected from halo, C₁-C₃ alkoxy, C₁-C₃ haloalkyl, and C₁-C₃ alkyl.

In embodiments, Ring A is a 5 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃ alkyl. In embodiments, Ring A is an aromatic 5 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃ alkyl. In embodiments, Ring A is a non-aromatic 5 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃alkyl.

In embodiments, Ring A is a 6 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃ alkyl. In embodiments, Ring A is an aromatic 6 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃ alkyl. In embodiments, Ring A is a non-aromatic 6 membered heterocyclic ring fused to the pyrimidine in Formula (II), comprising one heteroatom selected from N and O as ring members, optionally substituted with one group selected from halo, C₁-C₃ alkoxy, C₁-C₃ haloalkyl, and C₁-C₃alkyl.

In embodiments, Ring A is a 5-6 membered heterocyclic ring fused to the pyrimidine in Formula (II), comprising one or two heteroatoms selected from N and O as ring members. In embodiments, Ring A is an aromatic 5-6 membered heterocyclic ring fused to the pyrimidine in Formula (II), comprising one or two heteroatoms selected from N and O as ring members. In embodiments, Ring A is a non-aromatic 5-6 membered heterocyclic ring fused to the pyrimidine in Formula (II), comprising one or two heteroatoms selected from N and O as ring members.

In embodiments, Ring A is a 5 membered heterocyclic ring fused to the pyrimidine in Formula (II), comprising one or two heteroatoms selected from N and O as ring members. In embodiments, Ring A is an aromatic 5 membered heterocyclic ring fused to the pyrimidine in Formula (II), comprising one or two heteroatoms selected from N and O as ring members. In embodiments, Ring A is a non-aromatic 5 membered heterocyclic ring fused to the pyrimidine in Formula (II), comprising one or two heteroatoms selected from N and O as ring members.

In embodiments, Ring A is a 6 membered heterocyclic ring fused to the pyrimidine in Formula (II), comprising one or two heteroatoms selected from N and O as ring members. In embodiments, Ring A is an aromatic 6 membered heterocyclic ring fused to the pyrimidine in Formula (II), comprising one or two heteroatoms selected from N and O as ring members. In embodiments, Ring A is a non-aromatic 6 membered heterocyclic ring fused to the pyrimidine in Formula (II), comprising one or two heteroatoms selected from N and O as ring members.

In embodiments, Ring A is a 5 membered heterocyclic ring fused to the pyrimidine in Formula (II), comprising one heteroatom selected from N and O as ring members. In embodiments, Ring A is an aromatic 5 membered heterocyclic ring fused to the pyrimidine in Formula (II), comprising one heteroatom selected from N and O as ring members. In embodiments, Ring A is a non-aromatic 5 membered heterocyclic ring fused to the pyrimidine in Formula (II), comprising one heteroatom selected from N and O as ring members.

In embodiments, Ring A is a 6 membered heterocyclic ring fused to the pyrimidine in Formula (II), comprising one heteroatom selected from N and O as ring members. In embodiments, Ring A is an aromatic 6 membered heterocyclic ring fused to the pyrimidine in Formula (II), comprising one heteroatom selected from N and O as ring members. In embodiments, Ring A is a non-aromatic 6 membered heterocyclic ring fused to the pyrimidine in Formula (II), comprising one heteroatom selected from N and O as ring members.

In embodiments, Ring A is selected from pyrrole, furan, tetrahydrofuran, and tetrahydropyran. In embodiments, Ring A is selected from pyrrole and furan. In embodiments, Ring A is pyrrole. In embodiments, Ring A is furan. In embodiments, Ring A is tetrahydrofuran. In embodiments, Ring A is tetrahydropyran.

In embodiments, Ring A is pyrrole, furan, tetrahydrofuran, or tetrahydropyran, optionally substituted with one or two groups independently selected from halo, C₁-C₃ alkoxy, C₁-C₃ haloalkyl, and C₁-C₃ alkyl. In embodiments, Ring A is pyrrole, furan, tetrahydrofuran, or tetrahydropyran, optionally substituted with one group selected from halo, C₁-C₃ alkoxy, C₁-C₃ haloalkyl, and C₁-C₃ alkyl. In embodiments, Ring A is pyrrole, furan, tetrahydrofuran, or tetrahydropyran, optionally substituted with two groups independently selected from halo, C₁-C₃ alkoxy, C₁-C₃ haloalkyl, and C₁-C₃ alkyl.

In embodiments, Ring A is pyrrole, furan, tetrahydrofuran, or tetrahydropyran, optionally substituted with one or two groups independently selected from fluoro, chloro, bromo, iodo, methyl, ethyl, propyl, isopropyl, methoxy, ethoxy, and propoxy. In embodiments, Ring A is pyrrole, furan, tetrahydrofuran, or tetrahydropyran, optionally substituted with one group selected from fluoro, chloro, bromo, iodo, methyl, ethyl, propyl, isopropyl, methoxy, ethoxy, and propoxy. In embodiments, Ring A is pyrrole, furan, tetrahydrofuran, or tetrahydropyran, optionally substituted with two groups independently selected from fluoro, chloro, bromo, iodo, methyl, ethyl, propyl, isopropyl, methoxy, ethoxy, and propoxy.

In embodiments, Ring A is pyrrole optionally substituted with one group selected from fluoro, chloro, bromo, iodo, methyl, ethyl, propyl, isopropyl, methoxy, ethoxy, and propoxy. In embodiments, Ring A is pyrrole optionally substituted with methyl. In embodiments, Ring A is pyrrole optionally substituted with ethyl. In embodiments, Ring A is pyrrole optionally substituted with fluoro. In embodiments, Ring A is pyrrole optionally substituted with chloro. In embodiments, Ring A is pyrrole optionally substituted with methoxy. In embodiments, Ring A is pyrrole optionally substituted with ethoxy.

In embodiments, Ring A is absent, and pyrimidine is optionally substituted with one or two groups independently selected from halo, C₁-C₃ alkoxy, C₁-C₃ haloalkyl, and C₁-C₃ alkyl. In embodiments, Ring A is absent, and pyrimidine is optionally substituted with one group selected from halo, C₁-C₃ alkoxy, C₁-C₃ haloalkyl, and C₁-C₃ alkyl. In embodiments, Ring A is absent, and pyrimidine is optionally substituted with two groups independently selected from halo, C₁-C₃ alkoxy, C₁-C₃ haloalkyl, and C₁-C₃ alkyl.

In embodiments, Ring A is absent, and pyrimidine is optionally substituted with halo. In embodiments, Ring A is absent, and pyrimidine is optionally substituted with C₁-C₃ alkoxy. In embodiments, Ring A is absent, and pyrimidine is optionally substituted with C₁-C₃ haloalkyl. In embodiments, Ring A is absent, and pyrimidine is optionally substituted with C₁-C₃alkyl. In embodiments, Ring A is absent, and pyrimidine is unsubstituted.

In embodiments, Ring A is absent, and pyrimidine is optionally substituted with one group selected from fluoro, chloro, bromo, and iodo. In embodiments, Ring A is absent, and pyrimidine is optionally substituted with one group selected from methoxy, ethoxy, and propoxy. In embodiments, Ring A is absent, and pyrimidine is optionally substituted with one group selected from —CF₃, —CHF₂, —CH₂F, —CCl₃, —CHCl₂, and —CH₂Cl. In embodiments, Ring A is absent, and pyrimidine is optionally substituted with one group selected from methyl, ethyl, and propyl.

In embodiments, Ring A is absent, and pyrimidine is substituted with fluoro. In embodiments, Ring A is absent, and pyrimidine is substituted with methoxy. In embodiments, Ring A is absent, and pyrimidine is substituted with —CF₃. In embodiments, Ring A is absent, and pyrimidine is substituted with methyl.

In embodiments, provided herein is a compound of Formula (IIA):

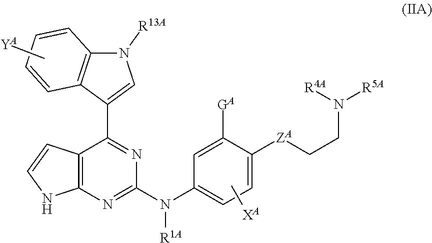

or an enantiomer, a mixture of enantiomers, a mixture of two or more diastereomers, a tautomer, a mixture of two or more tautomers, or an isotopic variant thereof; or a pharmaceutically acceptable salt thereof; wherein:
R^{13A} is selected from H and C₁-C₃ alkyl;
Y^{A} represents one or two optional substituents independently selected from halo, C₁-C₃ alkyl, C₁-C₃ alkoxy, and C₁-C₃ haloalkyl; and
wherein R^{1A}, R^{4A}, R^{5A}, Z^{A}, and G^{A} are each as defined herein.

In embodiments, R^{13A} is selected independently from H, methyl, ethyl, propyl, and isopropyl. In embodiments, R^{13A} is H. In embodiments, R^{13A} is methyl. In embodiments, R^{13A} is ethyl. In embodiments, R^{13A} is propyl. In embodiments, R^{13A} is isopropyl.

In embodiments, Y^{A} is one or two optional substituents independently selected from halo, C₁-C₃ alkyl, C₁-C₃ alkoxy, and C₁-C₃ haloalkyl. In embodiments, Y^{A} is one optional substituent selected from halo, C₁-C₃ alkyl, C₁-C₃ alkoxy, and C₁-C₃ haloalkyl. In embodiments, Y^{A} is two optional substituents independently selected from halo, C₁-C₃ alkyl, C₁-C₃alkoxy, and C₁-C₃ haloalkyl.

In embodiments, Y^{A} is one optional substituent which is halo. In embodiments, Y^{A} is one optional substituent which is chloro. In embodiments, Y^{A} is one optional substituent which is bromo. In embodiments, Y^{A} is one optional substituent which is iodo. In embodiments, Y^{A} is one optional substituent which is fluoro.

In embodiments, Y^{A} is one optional substituent which is C₁-C₃ alkyl. In embodiments, Y^{A} is one optional substituent which is C₁-C₃ alkoxy. In embodiments, Y^{A} is one optional substituent which is C₁-C₃ haloalkyl.

In embodiments, Y^{A} is one optional substituent which is methyl, ethyl, propyl, methoxy, ethoxy, propoxy, chloro, bromo, fluoro, or iodo. In embodiments, Y^{A} is one optional substituent which is methyl. In embodiments, Y^{A} is one optional substituent which is ethyl. In embodiments, Y^{A} is one optional substituent which is methoxy. In embodiments, Y^{A} is one optional substituent which is ethoxy.

In embodiments, Y^{A} is two optional substituents independently selected from chloro, bromo, iodo and fluoro.

In embodiments, Y^{A} is two optional substituents independently selected from chloro, bromo, iodo, fluoro, methyl, ethyl, propyl, methoxy, ethoxy, and propoxy.

In embodiments, Y^{A} is absent.

In an aspect, provided herein is a compound of Formula (III):

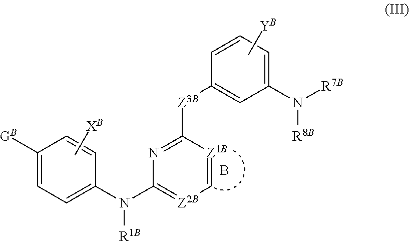

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Ring B is an optional 5 membered heteroaromatic ring fused to the ring containing Z^{2B} in Formula (III), comprising N or O as a ring member,
wherein Ring B is optionally substituted with one or two groups independently selected from halo, C₁-C₃ alkoxy, C₁-C₃ haloalkyl, C₁-C₃ alkyl, and —C(O)—R^{10B};
Z^{1B} is N when ring B is absent, and Z^{1B} is C when ring B is present;
Z^{2B} is N when ring B is present, and Z^{2B} is CR^{2B} when ring B is absent;
Z^{3B} is NR^{3B} or O;
G^{B} is a group of the formula —NR^{4B}—(CR^{1B})₂₋₃—NR^{5B}R^{6B} or G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B};
X^{B} represents one or two optional substituents independently selected from halo, C₁-C₃ alkyl, C₁-C₃ alkoxy, and C₁-C₃ haloalkyl;
Y^{B} represents one or two optional substituents independently selected from halo, C₁-C₃ alkyl, C₁-C₃ alkoxy, and C₁-C₃ haloalkyl;
R^{1B} is selected from H and C₁-C₃ alkyl;
R^{2B} is selected from H, halo, C₁-C₃ alkyl, and C₁-C₃ haloalkyl;
R^{3B} is selected from H and C₁-C₃ alkyl;
R^{4B} is selected from H, C₁-C₃ alkyl, and —C(O)—R^{10B};
R^{5B} is selected from H and C₁-C₃ alkyl;
R^{6B} is selected from H, C₁-C₃ alkyl, and —C(O)—R^{10B};
R^{7B} is selected from H and C₁-C₃ alkyl;
R^{8B} is selected from H, C₁-C₃ alkyl and —C(O)—R^{10B};
each R^{10B} is independently selected from H, C₁-C₃ alkyl, C₁-C₃ alkoxy, and C₁-C₃ haloalkyl.

In embodiments, X^{B} is one or two optional substituents independently selected from halo, C₁-C₃ alkyl, C₁-C₃ alkoxy, and C₁-C₃ haloalkyl. In embodiments, X^{B} is one optional substituent selected from halo, C₁-C₃ alkyl, C₁-C₃ alkoxy, and C₁-C₃ haloalkyl. In embodiments, X^{B} is two optional substituents independently selected from halo, C₁-C₃ alkyl, C₁-C₃ alkoxy, and C₁-C₃ haloalkyl.

In embodiments, X^{B} is one optional substituent which is halo. In embodiments, X B is one optional substituent which is chloro. In embodiments, X^{B} is one optional substituent which is bromo. In embodiments, X^{B} is one optional substituent which is iodo. In embodiments, X^{B} is one optional substituent which is fluoro.

In embodiments, X^{B} is one optional substituent which is C₁-C₃ alkyl. In embodiments, X^{B} is one optional substituent which is C₁-C₃ alkoxy. In embodiments, X^{B} is one optional substituent which is C₁-C₃ haloalkyl.

In embodiments, X^{B} is one optional substituent which is methyl, ethyl, or propyl.

In embodiments, X^{B} is two optional substituents independently selected from chloro, bromo, iodo and fluoro.

In embodiments, X^{B} is two optional substituents independently selected from chloro, bromo, iodo, fluoro, methyl, ethyl, propyl, methoxy, ethoxy, propoxy, and C₁-C₃ haloalkyl.

In embodiments, X^{B} is one substituent selected from methoxy and fluoro.

In embodiments, X^{B} is absent.

In embodiments, Y^{B} is one or two optional substituents independently selected from halo, C₁-C₃ alkyl, C₁-C₃ alkoxy, and C₁-C₃ haloalkyl. In embodiments, Y^{B} is one optional substituent selected from halo, C₁-C₃ alkyl, C₁-C₃ alkoxy, and C₁-C₃ haloalkyl. In embodiments, Y^{B} is two optional substituents independently selected from halo, C₁-C₃ alkyl, C₁-C₃ alkoxy, and C₁-C₃ haloalkyl.

In embodiments, Y^{B} is one optional substituent which is halo. In embodiments, y B is one optional substituent which is chloro. In embodiments, Y^{B} is one optional substituent which is bromo. In embodiments, Y^{B} is one optional substituent which is iodo. In embodiments, Y^{B} is one optional substituent which is fluoro.

In embodiments, Y^{B} is one optional substituent which is C₁-C₃ alkyl. In embodiments, Y^{B} is one optional substituent which is C₁-C₃ alkoxy. In embodiments, Y^{B} is one optional substituent which is C₁-C₃ haloalkyl.

In embodiments, Y^{B} is one optional substituent which is methyl, ethyl, or propyl.

In embodiments, Y^{B} is two optional substituents independently selected from chloro, bromo, iodo and fluoro.

In embodiments, Y^{B} is two optional substituents independently selected from chloro, bromo, iodo, fluoro, methyl, ethyl, propyl, methoxy, ethoxy, propoxy, and C₁-C₃ haloalkyl.

In embodiments, Y^{B} is absent.

In embodiments, Z^{3B} is selected from O and NR^{3B}. In embodiments, Z^{3B} is O. In embodiments, Z^{3B} is NR^{3B}.

In embodiments, Z^{3B} is NH. In embodiments, Z^{3B} is NCH₃. In embodiments, Z^{3B} is NCH₂CH₃. In embodiments, Z^{3B} is NCH₂CH₂CH₃. In embodiments, Z^{3B} is NCH(CH₃)₂.

In embodiments, Z^{2B} is N. In embodiments, Z^{2B} is CR^{2B}.

In embodiments, Z^{1B} is N. In embodiments, Z^{1B} is C.

In embodiments, ring B is a pyrrole or furan fused to a pyrimidine. In embodiments, ring B is a pyrrole fused to a pyrimidine. In embodiments, ring B is a furan fused to a pyrimidine.

In embodiments,

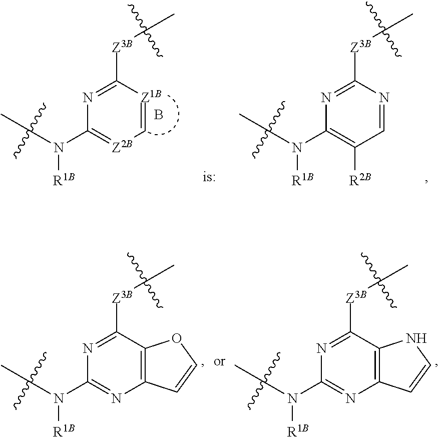

where Ring B is optionally substituted with one or two groups independently selected from halo, C₁-C₃ alkoxy, C₁-C₃ haloalkyl, C₁-C₃ alkyl, and —C(O)—R^{10B}.

In embodiments,

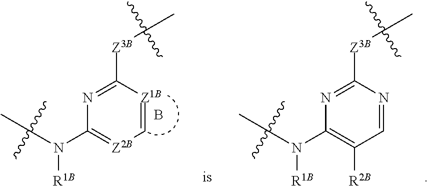

In embodiments,

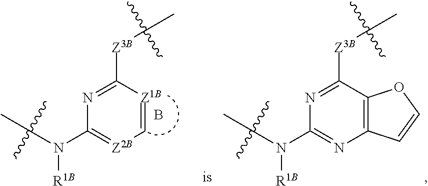

where Ring B is optionally substituted with one or two groups independently selected from halo, C₁-C₃ alkoxy, C₁-C₃ haloalkyl, C₁-C₃ alkyl, and —C(O)—R^{10B}. In embodiments,

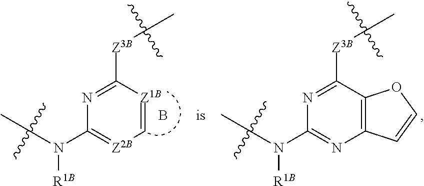

where Ring B is optionally substituted with one group selected from halo, C₁-C₃ alkoxy, C₁-C₃ haloalkyl, C₁-C₃ alkyl, and —C(O)—R^{10B}. In embodiments,

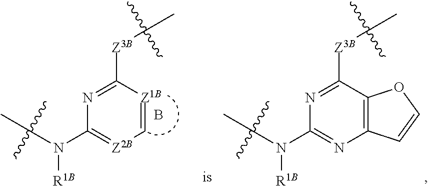

where Ring B is optionally substituted with two groups independently selected from halo, C₁-C₃ alkoxy, C₁-C₃ haloalkyl, C₁-C₃ alkyl, and —C(O)—R^{10B}.

In embodiments,

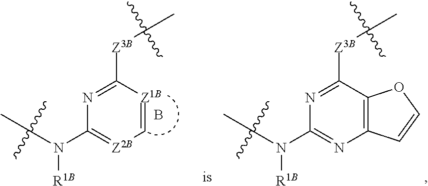

where Ring B is optionally substituted with one or two groups independently selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments,

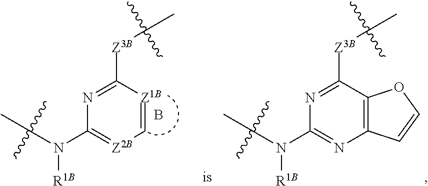

where Ring B is optionally substituted with one group selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments,

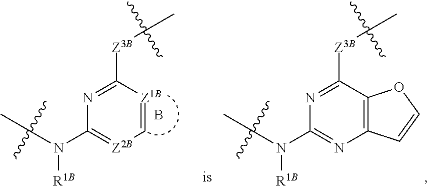

where Ring B is optionally substituted with two groups independently selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments,

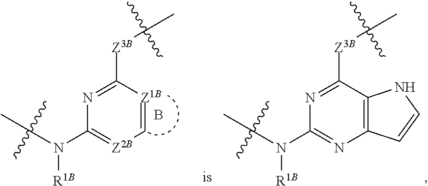

where Ring B is optionally substituted with one or two groups independently selected from halo, C₁-C₃ alkoxy, C₁-C₃ haloalkyl, C₁-C₃ alkyl, and —C(O)—R^{10B}. In embodiments,

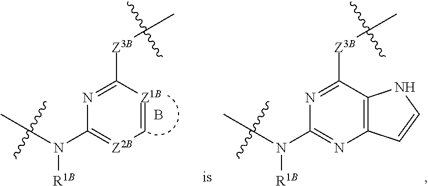

where Ring B is optionally substituted with one group selected from halo, C₁-C₃ alkoxy, C₁-C₃ haloalkyl, C₁-C₃ alkyl, and —C(O)—R^{10B}. In embodiments,

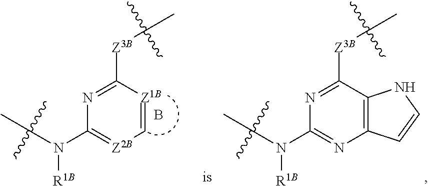

where Ring B is optionally substituted with two groups independently selected from halo, C₁-C₃ alkoxy, C₁-C₃ haloalkyl, C₁-C₃ alkyl, and —C(O)—R^{10B}.

In embodiments,

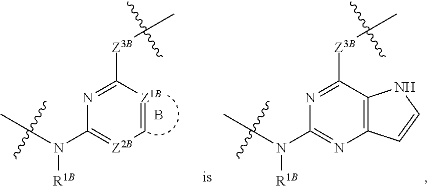

where Ring B is optionally substituted with one or two groups independently selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments,

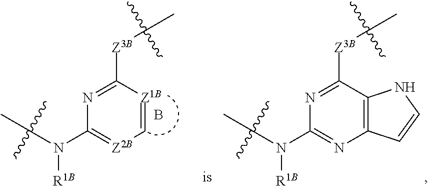

where Ring B is optionally substituted with one group selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments,

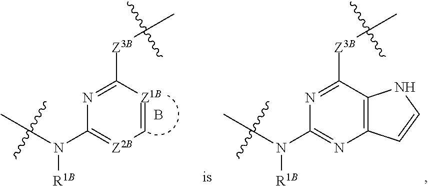

where Ring B is optionally substituted with two groups independently selected from fluoro, chloro, bromo, iodo, methyl, ethyl, propyl, isopropyl, methoxy, ethoxy, propoxy, methyl haloalkyl, ethyl haloalkyl, propyl haloalkyl, —C(O)—H, —C(O)—CH₃, —C(O)—CH₂CH₃, —C(O)—CH₂CH₂CH₃, —C(O)—CH(CH₃)₂, —C(O)—OCH₃, —C(O)—OCH₂CH₃, —C(O)—OCH₂CH₂CH₃, —C(O)—OCH(CH₃)₂, and —C(O)—C₁-C₃ haloalkyl.

In embodiments, R^{1B} is selected from H and C₁-C₃ alkyl. In embodiments, R^{1B} is H. In embodiments, R^{1B} is methyl. In embodiments, R^{1B} is ethyl. In embodiments, R^{1B} is propyl. In embodiments, R^{1B} is isopropyl.

In embodiments, R^{2B} is selected from H, halo, C₁-C₃ alkyl, and C₁-C₃ haloalkyl. In embodiments, R^{2B} is H. In embodiments, R^{2B} is halo. In embodiments, R^{2B} is C₁-C₃ alkyl. In embodiments, R^{2B} is C₁-C₃ haloalkyl.

In embodiments, R^{2B} is selected from H, fluoro, bromo, chloro, iodo, methyl, ethyl, propyl, isopropyl, and C₁-C₃ haloalkyl. In embodiments, R^{2B} is fluoro. In embodiments, R^{2B} is chloro. In embodiments, R^{2B} is iodo. In embodiments, R^{2B} is bromo. In embodiments, R^{2B} is methyl. In embodiments, R^{2B} is ethyl. In embodiments, R^{2B} ispropyl. In embodiments, R^{2B} is isopropyl. In embodiments, R^{2B} is C₁-C₃ haloalkyl.

In embodiments, R^{3B} is selected from H and C₁-C₃ alkyl. In embodiments, R^{3B} is H. In embodiments, R^{3B} is methyl. In embodiments, R^{3B} is ethyl. In embodiments, R^{3B} is propyl. In embodiments, R^{3B} is isopropyl.

In embodiments, R^{7B} is selected from H and C₁-C₃ alkyl. In embodiments, R^{7B} is selected from H, methyl, ethyl, propyl, and isopropyl. In embodiments, R^{7B} is H. In embodiments, R^{7B} is methyl. In embodiments, R^{7B} is ethyl. In embodiments, R^{7B} is propyl. In embodiments, R^{7B} is isopropyl.

In embodiments, R^{8B} is selected from H, C₁-C₃ alkyl and —C(O)—R^{10B}. In embodiments, R^{8B} is selected from H, methyl, ethyl, propyl, isopropyl, and —C(O)—R^{10B}. In embodiments, R^{8B} is H. In embodiments, R^{8B} is methyl. In embodiments, R^{8B} is ethyl. In embodiments, R^{8B} is propyl. In embodiments, R^{8B} is isopropyl. In embodiments, R^{8B} is —C(O)—R^{10B}. In embodiments, R^{8B} is —C(O)—H. In embodiments, R^{8B} is —C(O)—CH₃. In embodiments, R^{8B} is —C(O)—CH₂CH₃. In embodiments, R^{8B} is —C(O)—CH₂CH₂CH₃. In embodiments, R^{8B} is —C(O)—CH(CH₃)₂. In embodiments, R^{8B} is —C(O)—OCH₃. In embodiments, R^{8B} is —C(O)—OCH₂CH₃. In embodiments, R^{8B} is —C(O)—OCH₂CH₂CH₃. In embodiments, R^{8B} is —C(O)—OCH(CH₃)₂.

In embodiments, R^{10B} is selected from H, C₁-C₃ alkyl, C₁-C₃ alkoxy, and C₁-C₃ haloalkyl. In embodiments, R^{10B} is selected from H, methyl, ethyl, propyl, isopropyl, methoxy, ethoxy, propoxy, isopropoxy, and C₁-C₃ haloalkyl. In embodiments, R^{10B} is H. In embodiments, R^{10B} is methyl. In embodiments, R^{10B} is ethyl. In embodiments, R^{10B} is propyl. In embodiments, R^{10B} is isopropyl. In embodiments, R^{10B} is methoxy. In embodiments, R^{10B} is ethoxy. In embodiments, R^{10B} is propoxy. In embodiments, R^{10B} is isopropoxy. In embodiments, R^{10B} is C₁-C₃ haloalkyl.

In embodiments, R^{4B} is H. In embodiments, R^{4B} is methyl. In embodiments, R^{4B} is ethyl. In embodiments, R^{4B} is propyl. In embodiments, R^{4B} is isopropyl. In embodiments, R^{4B} is —C(O)—R^{10B}. In embodiments, R^{4B} is —C(O)—H. In embodiments, R^{4B} is —C(O)—CH₃. In embodiments, R^{4B} is —C(O)—CH₂CH₃. In embodiments, R^{4B} is —C(O)—CH₂CH₂CH₃. In embodiments, R^{4B} is —C(O)—CH(CH₃)₂. In embodiments, R^{4B} is —C(O)—OCH₃. In embodiments, R^{4B} is —C(O)—OCH₂CH₃. In embodiments, R^{4B} is —C(O)—OCH₂CH₂CH₃. In embodiments, R^{4B} is —C(O)—OCH(CH₃)₂.

In embodiments, R^{5B} is selected from H and C₁-C₃ alkyl. In embodiments, R^{5B} is H. In embodiments, R^{5B} is methyl. In embodiments, R^{5B} is ethyl. In embodiments, R^{5B} is propyl. In embodiments, R^{5B} is isopropyl.

In embodiments, R^{6B} is H. In embodiments, R^{6B} is methyl. In embodiments, R^{6B} is ethyl. In embodiments, R^{6B} is propyl. In embodiments, R^{6B} is isopropyl. In embodiments, R^{6B} is —C(O)—R^{10B}. In embodiments, R^{6B} is —C(O)—H. In embodiments, R^{6B} is —C(O)—CH₃. In embodiments, R^{6B} is —C(O)—CH₂CH₃. In embodiments, R^{6B} is —C(O)—CH₂CH₂CH₃. In embodiments, R^{6B} is —C(O)—CH(CH₃)₂. In embodiments, R^{6B} is —C(O)—OCH₃. In embodiments, R^{6B} is —C(O)—OCH₂CH₃. In embodiments, R^{6B} is —C(O)—OCH₂CH₂CH₃. In embodiments, R^{6B} is —C(O)—OCH(CH₃)₂.

In embodiments, G^{B} is a group of the formula —NR^{4B}—(CR^{1B})₂₋₃—NR^{5B}R^{6B}. In embodiments, G^{B} is a group of the formula —NR^{4B}—(CH₂)₂₋₃—NR^{5B}R^{6B}, wherein R^{4B}, R^{5B} and R^{6B} are each independently selected from methyl and ethyl. In embodiments, G^{B} is —N(CH₃)—(CH₂)₂₋₃—N(CH₃)₂. In embodiments, G^{B} is —N(CH₃)—(CH₂)₂—N(CH₃)₂. In embodiments, G^{B} is —N(CH₃)—(CH₂)₃—N(CH₃)₂.

In embodiments, G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B}.

In embodiments, G^{B} is a 6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B} In embodiments, G^{B} is a 6 membered saturated ring comprising one nitrogen atom as ring members, which is optionally substituted with one or two groups independently selected from halo, C₁-C₃ alkoxy, C₁-C₃ haloalkyl, C₁-C₃ alkyl, and —C(O)—R^{10B}. In embodiments, G^{B} is a 6 membered saturated ring comprising two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B}.

In embodiments, G^{B} is a group of the formula

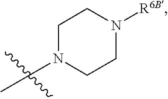

where R^{6B′} is selected from H, C₁-C₃ alkyl, and —C(O)—R^{10B}. In embodiments, G^{B} is a group of the formula

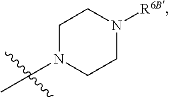

where R^{6B′} is H, methyl, or —C(O)CH₃. In embodiments, G^{B} is a group of the formula

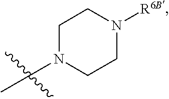

where R^{6B′} is H. In embodiments, G^{B} is a group of the formula

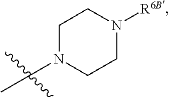

where R^{6B′} is methyl. In embodiments, G^{B} is a group of the formula

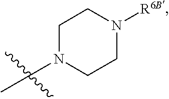

where R^{6B′} is —C(O)CH₃.

embodiments, G^{B} is a group of the formula

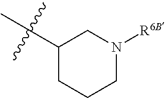

where R^{6B′} is selected from H, C₁-C₃ alkyl, and —C(O)—R^{10B}. In embodiments, G^{B} is a group of the formula

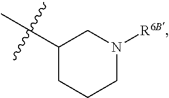

where R^{6B′} is H, methyl, or —C(O)CH₃. In embodiments, G^{B} is a group of the formula

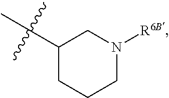

where R^{6B′} is H. In embodiments, G^{B} is a group of the formula

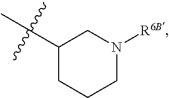

where R^{6B′} is methyl. In embodiments, G^{B} is a group of the formula

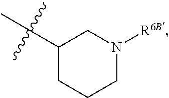

where R^{6B′} is —C(O)CH₃.

In an aspect, provided herein is a compound of Formula (IV):

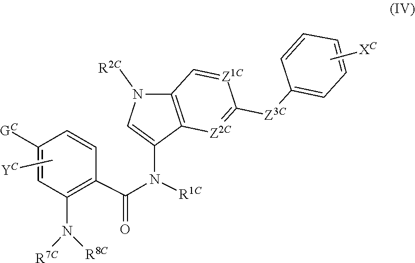

or an enantiomer, a mixture of enantiomers, a mixture of two or more diastereomers, a tautomer, a mixture of two or more tautomers, or an isotopic variant thereof; or a pharmaceutically acceptable salt thereof; wherein:
Z^{1C} and Z^{2C} are independently selected from N and CH;
Z^{3C} is selected from O, CH₂, and NR^{3C};
G^{C} is a group of the formula —NR^{4C}—(CR^{2C})₂₋₃—NR^{5C}R^{6C} or G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C};
X^{C} represents one or two optional substituents independently selected from halo, C₁-C₃ alkyl, C₁-C₃ alkoxy, and C₁-C₃ haloalkyl;
Y^{C} represents one or two optional substituents independently selected from halo, C₁-C₃ alkyl, C₁-C₃ alkoxy, and C₁-C₃ haloalkyl;
R^{1C} is selected from H and C₁-C₃ alkyl;
R^{2C} is selected from H and C₁-C₃ alkyl;
R^{3C} is selected from H and C₁-C₃ alkyl;
R^{4C} is selected from H, C₁-C₃ alkyl, and —C(O)—R^{10C};
R^{5C} is selected from H and C₁-C₃ alkyl;
R^{6C} is selected from H, C₁-C₃ alkyl, and —C(O)—R^{10C};
R^{7C} is selected from H, C₁-C₄ alkyl optionally substituted with C₁-C₃ alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl;
R^{8C} is selected from H, C₁-C₃ alkyl and —C(O)—R^{10C};
each R^{10C} is independently selected from H, C₁-C₃ alkyl, C₁-C₃ alkoxy, and C₁-C₃ haloalkyl.

In embodiments, X^{C} is one or two optional substituents independently selected from halo, C₁-C₃ alkyl, C₁-C₃ alkoxy, and C₁-C₃ haloalkyl. In embodiments, X^{C} is one optional substituent selected from halo, C₁-C₃ alkyl, C₁-C₃ alkoxy, and C₁-C₃ haloalkyl. In embodiments, X^{C} is two optional substituents independently selected from halo, C₁-C₃ alkyl, C₁-C₃alkoxy, and C₁-C₃ haloalkyl.

In embodiments, X^{C} is one optional substituent which is halo. In embodiments, X^{C} is one optional substituent which is chloro. In embodiments, X^{C} is one optional substituent which is bromo. In embodiments, X^{C} is one optional substituent which is iodo. In embodiments, X^{C} is one optional substituent which is fluoro.

In embodiments, X^{C} is one optional substituent which is C₁-C₃ alkyl. In embodiments, X^{C} is one optional substituent which is C₁-C₃ alkoxy. In embodiments, X^{C} is one optional substituent which is C₁-C₃ haloalkyl.

In embodiments, X^{C} is one optional substituent which is methyl, ethyl, or propyl.

In embodiments, X^{C} is two optional substituents independently selected from chloro, bromo, iodo and fluoro.

In embodiments, X^{C} is two optional substituents independently selected from chloro, bromo, iodo, fluoro, methyl, ethyl, propyl, methoxy, ethoxy, propoxy, and C₁-C₃ haloalkyl.

In embodiments, X^{C} is two substituents which are fluoro.

In embodiments, X^{C} is absent.

In embodiments, Y^{C} is one or two optional substituents independently selected from halo, C₁-C₃ alkyl, C₁-C₃ alkoxy, and C₁-C₃ haloalkyl. In embodiments, Y^{C} is one optional substituent selected from halo, C₁-C₃ alkyl, C₁-C₃ alkoxy, and C₁-C₃ haloalkyl. In embodiments, Y^{C} is two optional substituents independently selected from halo, C₁-C₃ alkyl, C₁-C₃alkoxy, and C₁-C₃ haloalkyl.

In embodiments, Y^{C} is one optional substituent which is halo. In embodiments, Y^{C} is one optional substituent which is chloro. In embodiments, Y^{C} is one optional substituent which is bromo. In embodiments, Y^{C} is one optional substituent which is iodo. In embodiments, Y^{C} is one optional substituent which is fluoro.

In embodiments, Y^{C} is one optional substituent which is C₁-C₃ alkyl. In embodiments, Y^{C} is one optional substituent which is C₁-C₃ alkoxy. In embodiments, Y^{C} is one optional substituent which is C₁-C₃ haloalkyl.

In embodiments, Y^{C} is one optional substituent which is methyl, ethyl, or propyl.

In embodiments, Y^{C} is two optional substituents independently selected from chloro, bromo, iodo and fluoro.

In embodiments, Y^{C} is two optional substituents independently selected from chloro, bromo, iodo, fluoro, methyl, ethyl, propyl, methoxy, ethoxy, propoxy, and C₁-C₃ haloalkyl.

In embodiments, Y^{C} is absent.

In embodiments, Z^{3C} is selected from O, CH₂, and NR^{3C}. In embodiments, Z^{3C} is O.

In embodiments, Z^{3C} is CH₂. In embodiments, Z^{3C} is NR^{3C}.

In embodiments, Z^{3C} is NH. In embodiments, Z^{3C} is NCH₃. In embodiments, Z^{3C} is NCH₂CH₃. In embodiments, Z^{3C} is NCH₂CH₂CH₃. In embodiments, Z^{3C} is NCH(CH₃)₂.

In embodiments, Z^{1C} and Z^{2C} are independently selected from N and CH. In embodiments, Z^{1C} and Z^{2C} are CH. In embodiments, Z^{1C} and Z^{2C} are N. In embodiments, Z^{1C} is CH and Z^{2C} is N. In embodiments, Z^{1C} is N and Z^{2C} is CH.

In embodiments, R^{2C} is selected from H and C₁-C₃ alkyl. In embodiments, R^{2C} is H. In embodiments, R^{2C} is methyl. In embodiments, R^{2C} is ethyl. In embodiments, R^{2C} is propyl. In embodiments, R^{2C} is isopropyl.

In embodiments, R^{1C} is selected from H and C₁-C₃ alkyl. In embodiments, R^{1C} is H. In embodiments, R^{1C} is methyl. In embodiments, R^{1C} is ethyl. In embodiments, R^{1C} is propyl. In embodiments, R^{1C} is isopropyl.

In embodiments, R^{3C} is selected from H and C₁-C₃ alkyl. In embodiments, R^{3C} is H, methyl, ethyl, propyl, or isopropyl. In embodiments, R^{3C} is H. In embodiments, R^{3C} is methyl. In embodiments, R^{3C} is ethyl. In embodiments, R^{3C} ispropyl. In embodiments, R^{3C} is isopropyl.

In embodiments, R^{8C} is selected from H, C₁-C₃ alkyl and —C(O)—R^{10C}. In embodiments, R^{8C} is selected from H, methyl, ethyl, propyl, isopropyl, and —C(O)—R^{10C} In embodiments, R^{8C} is selected from H and methyl.

In embodiments, R^{8C} is H. In embodiments, R^{8C} is methyl. In embodiments, R^{8C} is ethyl. In embodiments, R^{8C} is propyl. In embodiments, R^{8C} is isopropyl. In embodiments, R^{5C} is —C(O)—R^{10C}. In embodiments, R^{8C} is —C(O)—H. In embodiments, R^{8C} is —C(O)—CH₃. In embodiments, R^{8C} is —C(O)—CH₂CH₃. In embodiments, R^{8C} is —C(O)—CH₂CH₂CH₃. In embodiments, R^{8C} is —C(O)—CH(CH₃)₂. In embodiments, R^{8C} is —C(O)—OCH₃. In embodiments, R^{8C} is —C(O)—OCH₂CH₃. In embodiments, R^{8C} is —C(O)—OCH₂CH₂CH₃. In embodiments, R^{8C} is —C(O)—OCH(CH₃)₂.

In embodiments, R^{10C} is selected from H, C₁-C₃ alkyl, C₁-C₃ alkoxy, and C₁-C₃ haloalkyl. In embodiments, R^{10C} is selected from H, methyl, ethyl, propyl, isopropyl, methoxy, ethoxy, propoxy, isopropoxy, and C₁-C₃ haloalkyl. In embodiments, R^{10C} is H. In embodiments, R^{10C} is methyl. In embodiments, R^{10C} is ethyl. In embodiments, R^{10C} is propyl. In embodiments, R^{10C} is isopropyl. In embodiments, R^{10C} is methoxy. In embodiments, R^{10C} is ethoxy. In embodiments, R^{10C} is propoxy. In embodiments, R^{10C} is isopropoxy. In embodiments, R^{10C} is C₁-C₃ haloalkyl.

In embodiments, R^{7C} is selected from H, C₁-C₄ alkyl optionally substituted with C₁-C₃alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl.

In embodiments, R^{7C} is selected from H, methyl, ethyl, propyl, isopropyl, butyl, isobutyl, t-butyl, methyl optionally substituted with C₁-C₃ alkoxy, ethyl optionally substituted with C₁-C₃ alkoxy, propyl optionally substituted with C₁-C₃ alkoxy, butyl optionally substituted with C₁-C₃ alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl.

In embodiments, R^{7C} is C₂-C₄ alkyl substituted by methoxy. In embodiments, R^{7C} is methoxybutyl. In embodiments, R^{7C} is ethoxybutyl. In embodiments, R^{7C} is methoxypropyl. In embodiments, R^{7C} is ethoxypropyl. In embodiments, R^{7C} is a 5-6 membered heterocyclic group containing a heteroatom selected from N, O and S as a ring member. In embodiments, R^{7C} is a 6 membered heterocyclic group containing a heteroatom selected from N, O and S as a ring member. In embodiments, R^{7C} is a 6 membered heterocyclic group containing one O as a ring member. In embodiments, R^{7C} is a 6 membered heterocyclic group containing one N as a ring member. In embodiments, R^{7C} is a 6 membered heterocyclic group containing two N as ring members.

In embodiments, R^{7C} is piperazine. In embodiments, R^{7C} is piperidine. In embodiments, R^{7C} is tetrahydropyran. In embodiments, R^{7C} is tetrahydrofuran. In embodiments, R^{7C} is tetrahydropyran or tetrahydrofuran.

In embodiments, R^{7C} is piperazine substituted with one or two groups independently selected from halo, C₁-C₃ alkyl, C₁-C₃ alkoxy, and C₁-C₃ haloalkyl. In embodiments, R^{7C} is piperidine substituted with one or two groups independently selected from halo, C₁-C₃ alkyl, C₁-C₃ alkoxy, and C₁-C₃ haloalkyl. In embodiments, R^{7C} is tetrahydropyran substituted with one or two groups independently selected from halo, C₁-C₃ alkyl, C₁-C₃ alkoxy, and C₁-C₃ haloalkyl. In embodiments, R^{7C} is tetrahydrofuran substituted with one or two groups independently selected from halo, C₁-C₃ alkyl, C₁-C₃ alkoxy, and C₁-C₃ haloalkyl.

In embodiments, G^{C} is —NR^{4C}—(CR^{2C})₂₋₃NR^{5C}R^{6C}. In embodiments, R^{4C} is selected from H, C₁-C₃ alkyl, and —C(O)—R^{10C}. In embodiments, R^{4C} is selected from H, methyl, ethyl, propyl, isopropyl, and —C(O)—R^{10C}.

In embodiments, R^{4C} is H. In embodiments, R^{4C} is methyl. In embodiments, R^{4C} is ethyl. In embodiments, R^{4C} is propyl. In embodiments, R^{4C} is isopropyl. In embodiments, R^{4C} is —C(O)—R^{10C}. In embodiments, R^{4C} is —C(O)—H. In embodiments, R^{4C} is —C(O)—CH₃. In embodiments, R^{4C} is —C(O)—CH₂CH₃. In embodiments, R^{4C} is —C(O)—CH₂CH₂CH₃. In embodiments, R^{4C} is —C(O)—CH(CH₃)₂. In embodiments, R^{4C} is —C(O)—OCH₃. In embodiments, R^{4C} is —C(O)—OCH₂CH₃. In embodiments, R^{4C} is —C(O)—OCH₂CH₂CH₃. In embodiments, R^{4C} is —C(O)—OCH(CH₃)₂.

In embodiments, R^{5C} is selected from H and C₁-C₃ alkyl. In embodiments, R^{5C} is H. In embodiments, R^{5C} is methyl. In embodiments, R^{5C} is ethyl. In embodiments, R^{5C} is propyl. In embodiments, R^{5C} is isopropyl.

In embodiments, R^{6C} is H. In embodiments, R^{6C} is methyl. In embodiments, R^{6C} is ethyl. In embodiments, R^{6C} is propyl. In embodiments, R^{6C} is isopropyl. In embodiments, R^{6C} is —C(O)—R^{10C}. In embodiments, R^{6C} is —C(O)—H. In embodiments, R^{6C} is —C(O)—CH₃. In embodiments, R^{6C} is —C(O)—CH₂CH₃. In embodiments, R^{6C} is —C(O)—CH₂CH₂CH₃. In embodiments, R^{6C} is —C(O)—CH(CH₃)₂. In embodiments, R^{6C} is —C(O)—OCH₃. In embodiments, R^{6C} is —C(O)—OCH₂CH₃. In embodiments, R^{6C} is —C(O)—OCH₂CH₂CH₃. In embodiments, R^{6C} is —C(O)—OCH(CH₃)₂.

In embodiments, G^{C} is a group of the formula —NR^{4C}—(CR^{2C})₂₋₃—NR^{5C}R^{6C}. In embodiments, G^{C} is a group of the formula —NR^{4C}—(CH₂)₂₋₃—NR^{5C}R^{6C}, wherein R^{4C}, R^{5C} and R^{6C} are each selected independently from methyl and ethyl. In embodiments, G^{C} is —N(CH₃)—(CH₂)₂₋₃—N(CH₃)₂. In embodiments, G^{C} is —N(CH₃)—(CH₂)₂—N(CH₃)₂. In embodiments, G^{C} is —N(CH₃)—(CH₂)₃—N(CH₃)₂.

In embodiments, G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C}.

In embodiments, G^{C} is a 6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C}. In embodiments, G^{C} is a 6 membered saturated ring comprising one nitrogen atom as ring members, which is optionally substituted with one or two groups independently selected from halo, C₁-C₃ alkoxy, C₁-C₃ haloalkyl, C₁-C₃ alkyl, and —C(O)—R^{10C}. In embodiments, G^{C} is a 6 membered saturated ring comprising two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C}

In embodiments, G^{C} is a group of the formula

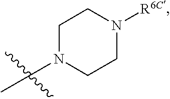

where R^{6C′} is selected from H, C₁-C₃ alkyl, and —C(O)—R^{10C}. In embodiments, G^{C} is a group of the formula

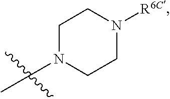

where R^{6C′} is H or methyl. In embodiments, G^{C} is a group of the formula

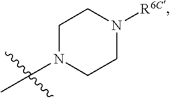

where R^{6C′} is H. In embodiments, G^{C} is a group of the formula

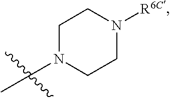

where R^{6C′} is methyl.

Also disclosed is an enantiomer, a mixture of enantiomers, a mixture of two or more diastereomers, a tautomer, a mixture of two or more tautomers, or an isotopic variant thereof of any of the compounds described herein.

### Pharmaceutical Compositions, Combinations, and Other Related Uses

In still another aspect, the present disclosure provides for a pharmaceutical composition comprising a compound described above admixed with at least one pharmaceutically acceptable carrier or excipient.

The above-described compounds can be used for any suitable purpose. For example, the present compounds can be used in therapy and/or testing.

In yet another aspect, the present disclosure provides for a method for treating and/or preventing a proliferation disorder, a cancer, or a tumor.

In yet another aspect, the present disclosure provides for a use of a compound described above for the manufacture of a medicament.

In yet another aspect, the present disclosure provides for a combination for treating and/or preventing a proliferation disorder in a subject, which combination comprises an effective amount of a compound described above, or a pharmaceutically acceptable salt thereof, and an effective amount of a second prophylactic or therapeutic agent for treating and/or preventing a proliferation disorder, a cancer, a tumor, an

In yet another aspect, the present disclosure provides for a method for modulating immune responses. In yet another aspect, the present disclosure provides for a method for treating and/or preventing a neurodegenerative disease. In yet another aspect, the present disclosure provides for a method for treating pain in cancer patients.

In yet another aspect, the present disclosure provides a method for inhibiting an activity of a cancer-associated tyrosine kinase such as EGFR kinase, FLT3 kinase, VEGFR, NTRK, RET, ALK, ROS/ROS1, DYRK1 and CK2 kinase in a subject or in a cell, which comprises contacting the tyrosine kinase with a compound of the disclosure, i.e., a compound of any of Formulas (I)-(IV), or any of the compounds in Table 1.

The present methods can be used for any suitable purpose. In some embodiments, the present methods can be used to treat and a proliferation disorder, a cancer, or a tumor.

In some embodiments, any of the compounds selected from the group consisting of compounds of Table 1 can be used in the compositions, combinations and methods of the disclosure.

### Formulations

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds according to any of Formulas I-IV as described herein are generally soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, etc. In one embodiment, the present disclosure provides formulations prepared by mixing a compound of any of formulas I-IV with a pharmaceutically acceptable carrier. In one aspect, the formulation may be prepared using a method comprising: a) dissolving a described compound in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of at least one exemplary compound of the present disclosure; optionally the compound may be administered in combination with one or more additional therapeutic agents, particularly therapeutic agents known to be useful for treating a proliferation disorder, a cancer, or a tumor afflicting the subject. Optionally the compound may be administered in combination with one or more additional therapeutic agents, particularly therapeutic agents known to be useful for treating neurodegenerative diseases.

The additional active ingredients may be administered in a separate pharmaceutical composition from at least one exemplary compound of the present disclosure or may be included with at least one exemplary compound of the present disclosure in a single pharmaceutical composition. The additional active ingredients may be administered simultaneously with, prior to, or after administration of at least one exemplary compound of the present disclosure.

In yet another aspect, the present disclosure provides a combination for treating and/or preventing a cell proliferation disorder in a subject, which combination comprises an effective amount of Formula (I), Formula (II), Formula (III), or Formula (IV), or any of the sub-formulae described herein, or a pharmaceutically acceptable salt thereof, and an effective amount of a second prophylactic or therapeutic agent for treating and/or preventing a cellular proliferation disorder, such as a cancer or tumor in a subject, preferably a subject having been diagnosed as in need of treatment for such disorder. Suitable second therapeutic agents for use in combination with the compounds of the disclosure include small molecule and antibody therapeutics useful to treat the same conditions to be treated with the compounds of Formula (I), Formula (II), Formula (III), or Formula (IV) or subformula thereof. Chemotherapeutic agents for use in such combinations include 5-fluorouracil, leucovorin, oxaliplatin, capecitabine, irinotecan, regorafenib, trifluridine, tipiracil, a drug that targets VEGF such as bevacizumab, ziv-aflibercept, or ramucirumab, or a drug that targets EGFR such as cetuximab or panitumumab.

In one aspect, the combination of the disclosure comprises a compound of Formula (I), Formula (II), Formula (III), or Formula (IV) or any subformula thereof, in combination with an immunooncology therapeutic agent, such as a PD-1 or PD-L1 inhibitor, or other known checkpoint inhibitors, that help the body's own immune system recognize and combat cancer cells. The checkpoint inhibitors assist the subject's immune system in recognizing and attacking abnormal cells, such as cancerous cells, and can significantly boost the efficacy of chemotherapies such as the compounds disclosed herein. Suitable checkpoint inhibitors include biologics as well as small-molecule therapeutics; examples of these include ipilimumab, nivolumab, atezolizumab, avelumab, pembrolizumab, tislelizumab, and durvalumab.

In yet another aspect, the present disclosure provides a combination for treating and/or preventing neurodegenerative diseases in a subject, which combination comprises an effective amount of Formula (I), Formula (II), Formula (III), or Formula (IV), or any of the sub-formulae described herein, or a pharmaceutically acceptable salt thereof, and an effective amount of a second prophylactic or therapeutic agent for treating and/or preventing neurodegenerative diseases in a subject, preferably a subject having been diagnosed as in need of treatment for such disorder.

### Methods of Using the Exemplary Compounds and Pharmaceutical Compositions Thereof.

The present disclosure also provides pharmaceutical compositions for the treatment and/or prevention of a proliferation disorder, a cancer, or a tumor, comprising any compound according to any of Formulas I-IV, or any of the compounds of the examples herein, particularly the compounds corresponding to Compound IDs in Table 1.

The present disclosure further provides pharmaceutical compositions for the treatment and/or prevention of neurodegenerative diseases, such as for example, Alzheimer's disease or Parkinson's disease, comprising any compound according to any of Formulas I-IV, or any of the compounds of the examples herein, particularly the compounds corresponding to Compound IDs in Table 1.

The present disclosure further provides pharmaceutical compositions for modulating immune responses and for pain relief in cancer patients, comprising any compound according to any of Formulas I-IV, or any of the compounds of the examples herein, particularly the compounds corresponding to Compound IDs in Table 1.

To practice the method of the present disclosure, compounds having formulas I-IV and pharmaceutical compositions thereof may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In addition, the compounds according to Formulas I-IV, or any of the compounds of the examples herein, may be administered alone or in combination with other therapeutic agents, e.g., anticancer agents, for the treatment of various proliferation disorder, cancer, or tumor, or agents for treating neurodegenerative diseases. Combination therapies according to the present disclosure comprise the administration of at least one exemplary compound of the present disclosure and at least one other pharmaceutically active ingredient. The active ingredient(s) and pharmaceutically active agents may be administered separately or together. The amounts of the active ingredient(s) and pharmaceutically active agent(s) and the relative timings of administration will be selected in order to achieve the desired combined therapeutic effect.

Chemotherapeutic agents for use in such pharmaceutical combinations and combination therapies include 5-fluorouracil, leucovorin, oxaliplatin, capecitabine, irinotecan, regorafenib, trifluridine, tipiracil, a drug that targets VEGF such as bevacizumab, ziv-aflibercept, or ramucirumab, or a drug that targets EGFR such as cetuximab or panitumumab. Suitable checkpoint inhibitors for use in such pharmaceutical combinations and combination therapies include biologics as well as small-molecule therapeutics; examples of these include ipilimumab, nivolumab, atezolizumab, avelumab, pembrolizumab, tislelizumab, and durvalumab.

### EXAMPLES

Compounds of the disclosure can be synthesized using known methods and starting materials, in view of the reaction schemes and examples herein.

### Syntheses of Aryl Chlorides/Intermediates

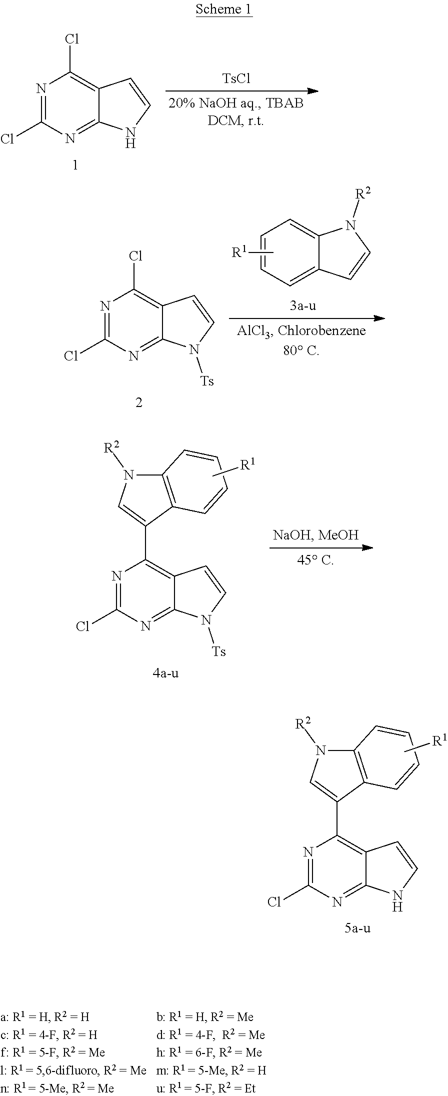

### Intermediate 4a: 2,4-dichloro-7-tosyl-7H-pyrrolo[2,3-d]pyrimidine (2)

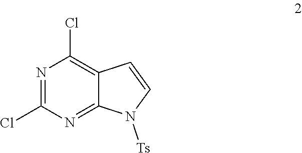 [SMILES: Cc1ccc(S(=O)(=O)n2ccc3c(Cl)nc(Cl)nc32)cc1]

NaOH (20% aqueous, 60 ml) was added to a mixture of 2,4-dichloro-7H-pyrrolo[2,3-d]pyrimidine 1 (10.5 g, 55.9 mmol), 4-tosyl chloride (12.4 g, 65.0 mmol) and tetrabutylammonium bromide (0.5 g, 1.6 mmol) in dichloromethane (120 ml) at room temperature. The reaction mixture was stirred for 0.5 h. The dichloromethane layer was separated and washed with water, then dried over Na₂SO₄ and filtered. The filtrate was concentrated to about 30 ml. Then heptane (50 ml) was added. The resulting mixture was stirred at 0° C. for 2 h and then filtered. The filter cake was washed with the solution of dichloromethane and heptane (1/3, v/v), dried to give the title compound 2 (16.5 g, yield 90.6% based on recovery of mother-liquor) as a white solid.

### 2-chloro-4-(1H-indol-3-yl)-7-tosyl-7H-pyrrolo[2,3-d]pyrimidine (4a)

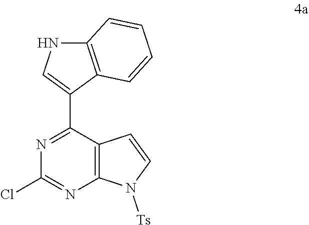 [SMILES: Cc1ccc(S(=O)(=O)n2ccc3c(-c4c[nH]c5ccccc45)nc(Cl)nc32)cc1]

To the mixture of 2 (3.48 g, 10.17 mmol) in PhCl (50 mL) were added AlCl3 (1.76 g, 13.22 mmol). The mixture was stirred at room temperature for 0.5 h, then indole 3a (1.67 g, 14.24 mmol) was added. The mixture was stirred at 85° C. for 12 h. The mixture was quenched with water, extracted with dichloromethane. The combined organic phase was concentrated, purified by flash column chromatography (petroleum ether/ethyl acetate=10/1-3/1) to give the 4a (2.2 g, 51.16% yield) as a brown solid. MS-ESI (M+H)⁺: 423.0.

### Intermediates 4b and 5b: 2-chloro-4-(1-methyl-1H-indol-3-yl)-7-tosyl-7H-pyrrolo[2,3-d]pyrimidine (4b)

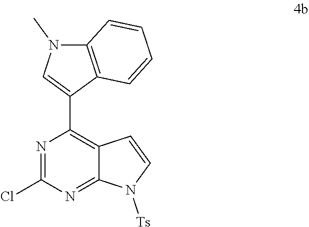 [SMILES: Cc1ccc(S(=O)(=O)n2ccc3c(-c4cn(C)c5ccccc45)nc(Cl)nc32)cc1]

AlCl₃ (7.15 g, 53.6 mmol) was added to a solution of 2,4-dichloro-7-tosyl-7H-pyrrolo[2,3-d]pyrimidine 2 (15.60 g, 45.6 mmol) in chlorobenzene (150 ml) at room temperature and the mixture was stirred for a while. 1-Methylindole 3b (6.95 g, 53.0 mmol) was then added dropwise and the mixture was heated to 80° C. for 19 h. The reaction was cooled with ice-bath and quenched by adding concentrated HCl (20 ml) and water (40 ml). After being stirred for 30 mins, the mixture was filtered and the resulting solid was collected. This solid was then stirred in saturated Na₂CO₃ for 30 mins and collected by filtration. After being dried for 1 h, the solid was then stirred in refluxing ethyl acetate (35 ml) for 30 min. The mixture was allowed to cool to 0° C. at which point heptane (20 ml) was added. The mixture was stirred for another 30 mins, and then filtered. The filter cake was dried to give the title compound 4b (15.0 g, yield 75.3%) as a light yellow solid.

### 2-chloro-4-(1-methyl-1H-indol-3-yl)-7H-pyrrolo[2,3-d]pyrimidine (5b)

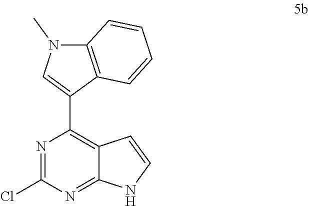 [SMILES: Cn1cc(-c2nc(Cl)nc3[nH]ccc23)c2ccccc21]

NaOH (20.20 g, 50.5 mmol) was added to a solution of 2-chloro-4-(1-methyl-1H-indol-3-yl)-7-tosyl-7H-pyrrolo[2,3-d]pyrimidine 4b (10.10 g, 23.1 mmol) in methanol (120 ml). The reaction was stirred for 1 h and then cooled to 15° C. Precipitate appeared in the reaction and then the methanol (˜20 ml) was partly evaporated in vacuo. The mixture was then cooled to 0° C. at which point ice-water (100 ml) was added. The resulting mixture was stirred for 2 h and filtered. The filter cake was washed with water for 3 times, dried to give the title compound 5b (4.5 g, yield 68.8%) as a light yellow solid.

### Intermediates 4c and 5c: 2-chloro-4-(4-fluoro-1H-indol-3-yl)-7-tosyl-7H-pyrrolo[2,3-d]pyrimidine (4c)

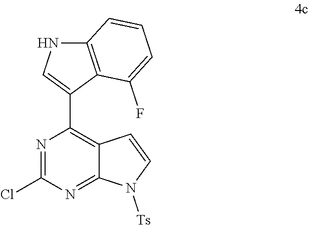 [SMILES: Cc1ccc(S(=O)(=O)n2ccc3c(-c4c[nH]c5cccc(F)c45)nc(Cl)nc32)cc1]

AlCl₃ (1.92 g, 0.014 mol) was added to a stirred solution of 2,4-dichloro-7-tosyl-7H-pyrrolo[2,3-d]pyrimidine 2 (3.45 g, 0.01 mol) in chlorobenzene (50 ml) at room temperature. The resulting solution was stirred for 30 minutes and then the solution of 4-fluoro-1H-indole 3c (1.81 g, 13.4 mmol) in chlorobenzene (5 ml) was added dropwise to the reaction mixture at room temperature. The resulting mixture was heated at 80° C. for 4 h. Then the reaction mixture was allowed to cool down and quench with water (20 ml) and HCl (1M, 30 ml). The mixture was stirred for another 0.5 h, and then was filtered to afford the title compound 4c, which was used in the next step without further purification.

### 2-chloro-4-(4-fluoro-1H-indol-3-yl)-7H-pyrrolo[2,3-d]pyrimidine (5c)

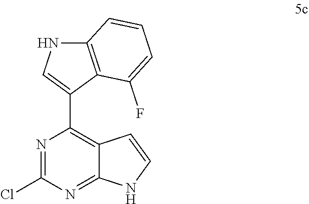 [SMILES: Fc1cccc2[nH]cc(-c3nc(Cl)nc4[nH]ccc34)c12]

NaOH (6.1 g, 0.15 mol) was added separately to the solution of above 4c in methanol. And then the mixture was stirred for 0.5 h at 45° C. Then the reaction mixture was allowed to cool down and quench with water (100 ml). The mixture was stirred for 0.5 h and then filtered. The filter cake was washed with water and then stirred in ethyl acetate (20 ml) over night. The resulting mixture was filtered and the filter cake was dried in vacuo to give the title compound 5c (0.9 g, 31.5% over two steps).

### Intermediate 4d: 2-chloro-4-(4-fluoro-1-methyl-1H-indol-3-yl)-7-tosyl-7H-pyrrolo[2,3-d]pyrimidine (4d)

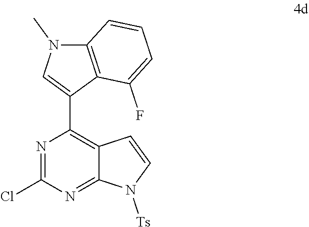 [SMILES: Cc1ccc(S(=O)(=O)n2ccc3c(-c4cn(C)c5cccc(F)c45)nc(Cl)nc32)cc1]

AlCl₃ (630.5 mg, 4.72 mmol) was added to a stirred solution of 2,4-dichloro-7-tosyl-7H-pyrrolo[2,3-d]pyrimidine 2 (1.3301 g, 3.89 mmol) in chlorobenzene (20 ml) at room temperature. The resulting solution was stirred a while and then 4-fluoro-1-methyl-1H-indole 3d (1.0833 g, 7.26 mmol) was added dropwise to the reaction mixture at room temperature. The resulting mixture was heated at 80° C. for 21 h. Then the reaction mixture was allowed to cool down and pour into water (150 ml). The mixture was stirred for another 1 h, and then was filtered to afford part of the title compound 4d (0.22 g). The filtrate was extracted with dichloromethane. The organic layers were combined and concentrated under reduced pressure. The residue was purified by flash column chromatography (dichloromethane/methanol=10/1) to give another part of 4d (730 mg, totally 950 mg, yield 53.7%) as a gray solid.

### Intermediate 4f: 2-chloro-4-(5-fluoro-1-methyl-1H-indol-3-yl)-7-tosyl-7H-pyrrolo[2,3-d]pyrimidine (4f)

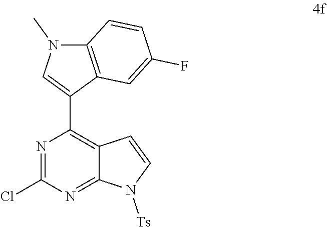 [SMILES: Cc1ccc(S(=O)(=O)n2ccc3c(-c4cn(C)c5ccc(F)cc45)nc(Cl)nc32)cc1]

AlCl₃ (1.75 g, 0.013 mol) was added to a stirred solution of 2,4-dichloro-7-tosyl-7H-pyrrolo[2,3-d]pyrimidine 2 (3.43 g, 0.01 mol) in toluene (50 ml) at room temperature. The resulting solution was stirred for 30 minutes and then the solution of 5-fluoro-1-methyl-1H-indole 3f (2.3 g, 0.15 mol) in toluene (5 ml) was added dropwise to the reaction mixture at room temperature. The resulting mixture was heated at 80° C. for 4 h. Then the reaction mixture was allowed to cool down and quench with water (10 ml) and HCl (1M, 30 ml). The mixture was stirred for another 0.5 h, and then was filtered. The filter cake was dried in vacuo to give the title compound 4f (4.0 g, yield 58.7%).

### Intermediate 4h: 2-chloro-4-(6-fluoro-1-methyl-1H-indol-3-yl)-7-tosyl-7H-pyrrolo[2,3-d]pyrimidine (4h)

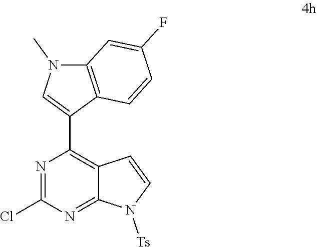 [SMILES: Cc1ccc(S(=O)(=O)n2ccc3c(-c4cn(C)c5cc(F)ccc45)nc(Cl)nc32)cc1]

AlCl₃ (1.75 g, 0.013 mol) was added to a stirred solution of 2,4-dichloro-7-tosyl-7H-pyrrolo[2,3-d]pyrimidine 2 (3.43 g, 0.01 mol) in chlorobenzene (50 ml) at room temperature. The resulting solution was stirred for 30 minutes and then the solution of 6-fluoro-1-methyl-1H-indole 3h (2.3 g, 0.15 mol) in chlorobenzene (5 ml) was added dropwise to the reaction mixture at room temperature. The resulting mixture was heated at 85° C. for 4 h. Then the reaction mixture was allowed to cool down and quench with water (10 ml) and HCl (1M, 30 ml). The mixture was stirred for another 0.5 h, and then was filtered. The filter cake was dried in vacuo to give the title compound 4h (3.5 g, yield 76.9%).

### Intermediate 41: 2-chloro-4-(5,6-difluoro-1-methyl-1H-indol-3-yl)-7-tosyl-7H-pyrrolo[2,3-d]pyrimidine (4i)

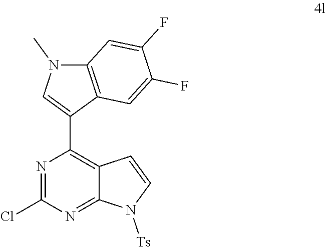 [SMILES: Cc1ccc(S(=O)(=O)n2ccc3c(-c4cn(C)c5cc(F)c(F)cc45)nc(Cl)nc32)cc1]

AlCl₃ (1.75 g, 0.013 mol) was added to a stirred solution of 2,4-dichloro-7-tosyl-7H-pyrrolo[2,3-d]pyrimidine 2 (3.41 g, 0.01 mol) in chlorobenzene (50 ml) at room temperature. The resulting solution was stirred for 30 minutes and then the solution of 5,6-difluoro-1-methyl-1H-indole 31 (2.05 g, 0.15 mol) in chlorobenzene (5 ml) was added dropwise to the reaction mixture at room temperature. The resulting mixture was heated at 80° C. for 4 h. Then the reaction mixture was allowed to cool down and quench with water (20 ml) and HCl (1M, 30 ml). The mixture was stirred for another 0.5 h, and then was filtered. The filter cake was dried in vacuo to give the title compound 41 (3.8 g, yield 80.51%).

### Intermediate 4m: 2-chloro-4-(5-methyl-1H-indol-3-yl)-7-tosyl-7H-pyrrolo[2,3-d]pyrimidine (4m)

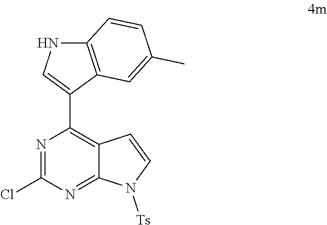 [SMILES: Cc1ccc(S(=O)(=O)n2ccc3c(-c4c[nH]c5ccc(C)cc45)nc(Cl)nc32)cc1]

AlCl₃ (1.73 g, 12.97 mmol) was added to a stirred solution of 2,4-dichloro-7-tosyl-7H-pyrrolo[2,3-d]pyrimidine 2 (3.45 g, 10 mmol) in chlorobenzene (50 ml) at room temperature. The resulting solution was stirred for 30 minutes and then 5-methyl-1H-indole 3m (1.51 g, 11.46 mmol) was added dropwise to the reaction solution at room temperature. The resulting mixture was heated at 80° C. for 4 h. Then the reaction mixture was allowed to cool down and quench with water (20 ml) and HCl (1M, 10 ml). The mixture was stirred for another 0.5 h, and then was filtered. The filter cake was stirred in water (50 ml) for 0.5 h. After filtration, the filter cake was stirred in ethyl acetate (20 ml) overnight. Then the solid was collected by filtration and dried in vacuo to give the title product 4m (2.1 g, yield 48.06%).

### Intermediate 4n: 2-chloro-4-(1,5-dimethyl-1H-indol-3-yl)-7-tosyl-7H-pyrrolo[2,3-d]pyrimidine (4n)

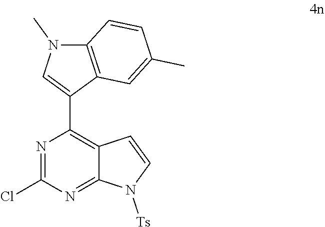 [SMILES: Cc1ccc(S(=O)(=O)n2ccc3c(-c4cn(C)c5ccc(C)cc45)nc(Cl)nc32)cc1]

AlCl₃ (1.75 g, 0.013 mol) was added to a stirred solution of 2,4-dichloro-7-tosyl-7H-pyrrolo[2,3-d]pyrimidine 2 (3.45 g, 0.01 mol) in toluene (50 ml) at room temperature. The resulting solution was stirred for 30 minutes and then the solution of 1,5-dimethyl-1H-indole 3n (2.3 g, 0.15 mol) in toluene (5 ml) was added dropwise to the reaction mixture at room temperature. The resulting mixture was heated at 80° C. for 4 h. Then the reaction mixture was allowed to cool down and quench with water (10 ml) and HCl (1M, 30 ml). The mixture was stirred for another 0.5 h, and then was filtered. The filter cake was dried in vacuo to give the title compound 4n (4.2 g, yield 44.3%).

### Intermediate 4u: 2-chloro-4-(1-ethyl-5-fluoro-1H-indol-3-yl)-7-tosyl-7H-pyrrolo[2,3-d]pyrimidine (4u)

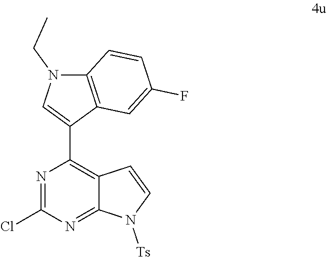 [SMILES: CCn1cc(-c2nc(Cl)nc3c2ccn3S(=O)(=O)c2ccc(C)cc2)c2cc(F)ccc21]

AlCl₃ (597 mg, 4.48 mmol) was added into a solution of 2 (1.01 g, 2.95 mmol) in PhCl (20 mL) followed by stirring at 20° C. for 0.5 h. A solution of 1-ethyl-5-fluoro-1H-indole 3u (806 mg, 4.94 mmol) in PhCl (5 mL) was added to the reaction. The reaction mixture was stirred at 80° C. for 16 h. The reaction was cooled to room temperature, and quenched with HCl (4N, 15 mL) followed by stirring for 1 h. The resulting mixture was filtered and the filter cake was washed with ethyl acetate (10 mL), dried under reduced pressure to afford 4u (1.01 g, yield 73.0%) as a white solid.

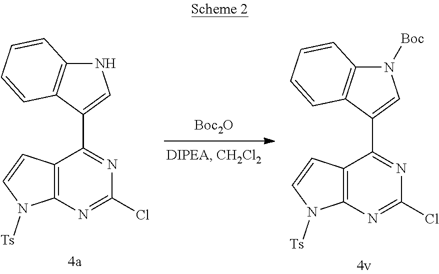 [SMILES: "Boc2O" ; Cc1ccc(S(=O)(=O)n2ccc3c(-c4c[nH]c5ccccc45)nc(Cl)nc32)cc1 ; Cc1ccc(S(=O)(=O)n2ccc3c(-c4cn(C(=O)OC(C)(C)C)c5ccccc45)nc(Cl)nc32)cc1]

### Intermediate 4v: tert-butyl 3-(2-chloro-7-tosyl-7H-pyrrolo[2,3-d]pyrimidin-4-yl)-1H-indole-1-carboxylate (4v)

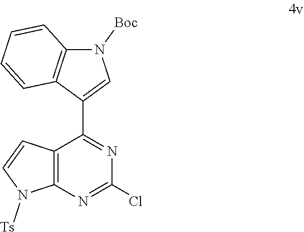 [SMILES: Cc1ccc(S(=O)(=O)n2ccc3c(-c4cn(C(=O)OC(C)(C)C)c5ccccc45)nc(Cl)nc32)cc1]

To a solution of 4a (1 g, 2.36 mmol) and DIPEA (0.915 g, 7.98 mmol) in dichloromethane (50 mL) were added Boc₂O (0.619 g, 2.84 mmol). The mixture was stirred at 20° C. for 8 h. The mixture was concentrated, purified by flash column chromatography (dichloromethane/methanol=100/1-10/1) to give the 4v (0.8 g, 64.69% yield) as a white solid. MS-ESI (M+H)⁺: 523.1.

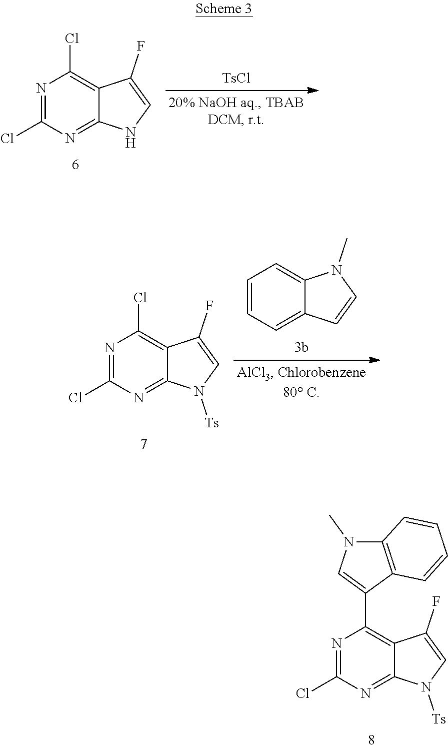

### Intermediate 8: 2,4-dichloro-5-fluoro-7-tosyl-7H-pyrrolo[2,3-d]pyrimidine (7)

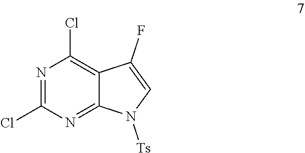 [SMILES: Cc1ccc(S(=O)(=O)n2cc(F)c3c(Cl)nc(Cl)nc32)cc1]

Synthesis of 7 was similar to that of 2. Using 6 (2.05 g, 10.0 mmol), 4-tosyl chloride (2.21 g, 11.6 mmol), NaOH (20% aqueous, 12 ml) and tetrabutylammonium bromide (0.02 g, 0.062 mmol) to give 7 as a white solid (3.2 g, yield 88.9%).

### 2-chloro-5-fluoro-4-(1-methyl-1H-indol-3-yl)-7-tosyl-7H-pyrrolo[2,3-d]pyrimidine (8)

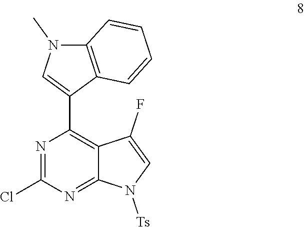 [SMILES: Cc1ccc(S(=O)(=O)n2cc(F)c3c(-c4cn(C)c5ccccc45)nc(Cl)nc32)cc1]

Synthesis of 8 was similar to that of 4b. The reaction was repeated for three times by using 7 (2.13 g, 10.0 mmol) totally. And all the crudes were combined and purified by flash column chromatography to give 8 as a gray solid (2.5 g, yield 92.9%).

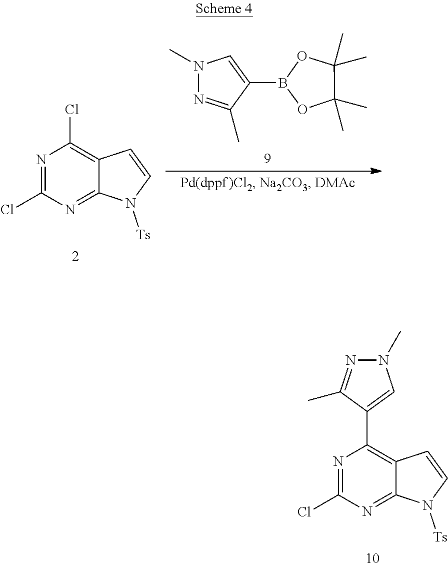

### Intermediate 10: 2-chloro-4-(1,3-dimethyl-1H-pyrazol-4-yl)-7-tosyl-7H-pyrrolo[2,3-d]pyrimidine (10)

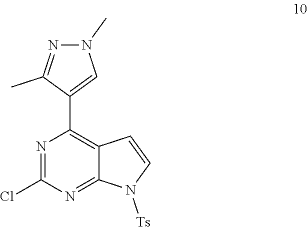 [SMILES: Cc1ccc(S(=O)(=O)n2ccc3c(-c4cn(C)nc4C)nc(Cl)nc32)cc1]

A mixture of 2 (1.85 g, 5.5 mmol), 1,3-dimethyl-4-(4,4,5,5-tetramethyl-1,3,2-dioxaborolan-2-yl)-1H-pyrazole 9 (1.21 g, 5.5 mmol), Pd(dppf)Cl₂ (0.19 g, 0.25 mmol) and Na₂CO₃ (2M a.q., 10 ml, 20 mmol) in N,N-dimethylacetamide (25 mL) was stirred under N₂ at 80° C. for 3 h. After cooling to room temperature, the reaction mixtures was filtered through Celite® and the filtrates were extracted with ethyl acetate. The organic layer was separated, washed with water and concentrated under reduced pressure to dryness. The crude was purified by flash column chromatography (eluting with ethyl acetate) to give the title compound 10 (1.2 g, yield 54.0%) as a white solid.

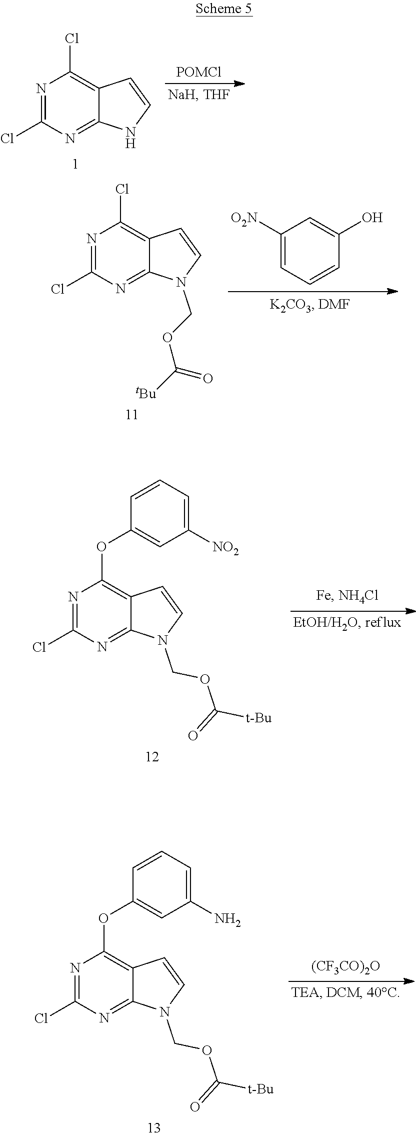

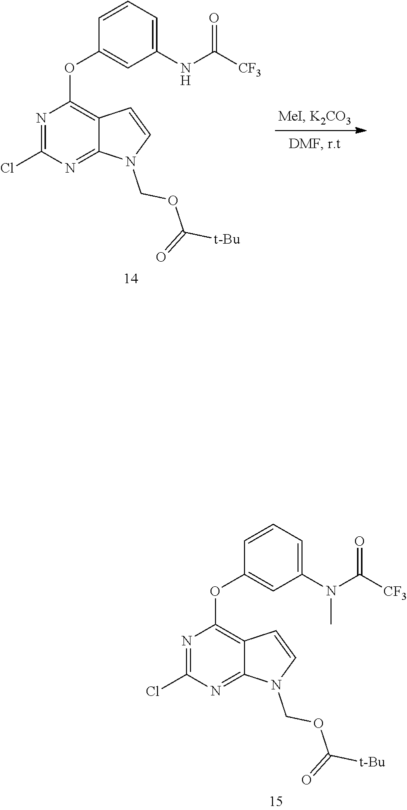

### Intermediate 15: (2, 4-dichloro-7H-pyrrolo[2,3-d]pyrimidin-7-yl)methyl Pivalate (11)

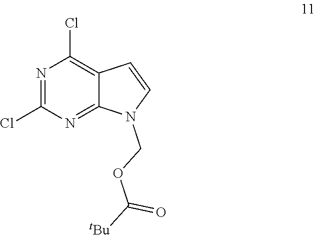 [SMILES: CC(C)(C)C(=O)OCn1ccc2c(Cl)nc(Cl)nc21]

NaH (80%, 35.4 g, 1.17 mol) was added slowly to a solution of 2, 4-dichloro-7H-pyrrolo[2,3-d]pyrimidine 1 (200.3 g, 1.06 mol) in THF (2.3 L), maintaining the temperature between −5˜−10° C. The mixture was stirred for another 15 min until the evolution of hydrogen ceased. A solution of POMCl (189.6 g, 1.2 mol) in THF (0.7 L) was then added to the mixture within 30 min. The reaction mixture was allowed to warm to room temperature and stirred for 3-4 h. When HPLC indicated that 1 had been consumed, the reaction mixture was filtered through Celite®, washed with ethyl acetate (0.5 L). All the filtrate was combined and concentrated under vacuum. The residue was then diluted with ethyl acetate (2.5-3 L), washed by water (1 L*2) and brine (1 L). The ethyl acetate layer was separated and dried under vacuum to afford crude 11 as a yellow solid, which was used directly for the next step.

### (2-chloro-4-(3-nitrophenoxy)-7H-pyrrolo[2,3-d]pyrimidin-7-yl)methyl Pivalate (12)

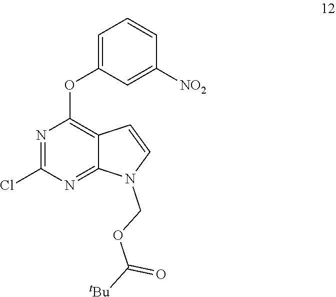 [SMILES: CC(C)(C)C(=O)OCn1ccc2c(Oc3cccc([N+](=O)[O-])c3)nc(Cl)nc21]

To a mixture of pyrimidine 11 (320 g, 1.06 mol) and 3-nitro phenol (146 g, 1.04 mol) in DMF (2.5 L) was added K₂CO₃ (290 g, 2.1 mol). The reaction mixture was stirred at room temperature for 5˜6 h. When HPLC indicated that 2 had been consumed, K₂CO₃ was then filtered through Celite®, wash with ethyl acetate (0.5 L). And the filtrate was dilute with ethyl acetate (3 L) and water (2 L). The ethyl acetate layer was separated and washed sequentially with water (1 L*3), brine (1 L*3) and water (1 L*1). Extraction of the aqueous phase was repeated if necessary. The organic layer was dried in vacuo to afford the crude 12 (342 g, 80% yield) which was then purified by crystallization in petroleum ether/ethyl acetate system (petroleum ether/ethyl acetate=25/3, v/v; 5.6 ml for 1 g crude 12). LC-MS: m/z 405.2 [M+H]⁺. ¹H NMR (500 MHz, CDCl₃): δ ppm 8.17-8.14 (m, 1H), 8.14-8.13 (m, 1H), 7.63-7.62 (m, 2H), 7.37 (d, J=3.7 Hz, 1H), 6.62 (d, J=3.7 Hz, 1H), 6.18 (s, 2H), 1.17 (s, 9H); ¹³C NMR (125 MHz, CDCl₃): δ ppm 178.4, 161.6, 154.6, 152.7, 152.5, 149.1, 130.3, 128.6, 128.2, 120.8, 117.4, 104.8, 100.3, 65.9, 39.0, 27.0 (*3).

### (4-(3-aminophenoxy)-2-chloro-7H-pyrrolo[2,3-d]pyrimidin-7-yl)methyl Pivalate (13)

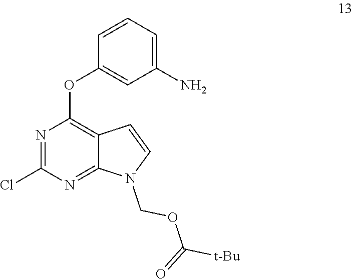 [SMILES: CC(C)(C)C(=O)OCn1ccc2c(Oc3cccc(N)c3)nc(Cl)nc21]

To a solution of (2-chloro-4-(3-nitrophenoxy)-7H-pyrrolo[2,3-d]pyrimidin-7-yl) methyl pivalate 12 (2.3 g, 5.68 mmol) in ethanol (25 mL) and H₂O (5 mL) was added iron powder (1.6 g, 28.65 mmol) and NH₄Cl (2.0 g, 38.53 mmol). The mixture was stirred at 90° C. for 1 h. After cooling to room temperature, the mixture was filtered off and the filtrate was concentrated under reduced pressure. The residue diluted with ethyl acetate (100 mL). The organic phase was washed with H₂O (40 mL*2), dried with Na₂SO₄. The organic phase was concentrated under reduced pressure to give the crude product 13 (2.0 g, yield 93.9%) as a gray solid, which was used in the next step without further purification.

### (2-chloro-4-(3-(2,2,2-trifluoroacetamido)phenoxy)-7H-pyrrolo[2,3-d]pyrimidin-7-yl)methyl Pivalate (14)

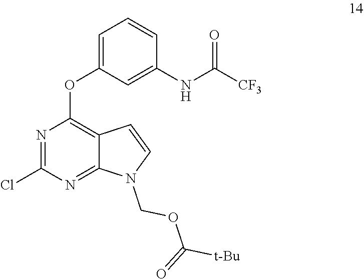 [SMILES: CC(C)(C)C(=O)OCn1ccc2c(Oc3cccc(NC(=O)C(F)(F)F)c3)nc(Cl)nc21]

A solution of 13 (1.69 g, 4.51 mmol) and Et₃N (3.64 g, 36.00 mmol) in dichloromethane (15 mL) was stirred at 0° C. for 10 min. Then trifluoroacetic anhydride (1.65 g, 7.86 mmol) was added dropwise and the mixture was stirred at 40° C. for another 2 h. The reaction mixture was concentrated under reduced pressure. The residue was dissolved in dichloromethane (50 mL), and washed with H₂O (20 mL*2), dried with Na₂SO₄. After concentration, the residue was purified by flash column chromatography (petroleum ether/ethyl acetate=7/1) to give the product 14 (1.56 g, yield 73.5%) as an off-white solid.

### (2-chloro-4-(3-(2,2,2-trifluoro-N-methylacetamido)phenoxy)-7H-pyrrolo[2,3-d]pyrimidin-7-yl)methyl Pivalate (15)

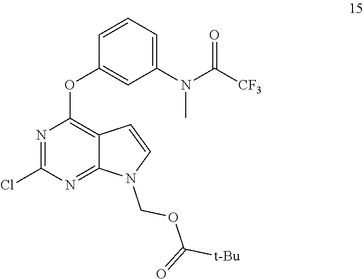 [SMILES: CN(C(=O)C(F)(F)F)c1cccc(Oc2nc(Cl)nc3c2ccn3COC(=O)C(C)(C)C)c1]

To a solution of 14 (1.55 g, 3.29 mmol) in DMF (12 mL), K₂CO₃ (0.88 g, 6.38 mmoL) was added. The mixture was stirred at room temperature for 10 min followed by addition of Mel (0.65 g, 4.58 mmol). The mixture was stirred at 45° C. for 2.5 h. After cooling to room temperature, the mixture was diluted with ethyl acetate (50 mL). The organic phase was washed with H₂O (20 mL*2), dried with Na₂SO₄. After concentration, the residue was purified by flash column chromatography (petroleum ether/ethyl acetate=10/1) to give the product 15 (1.45 g, yield 90.8%) as an off-white solid.

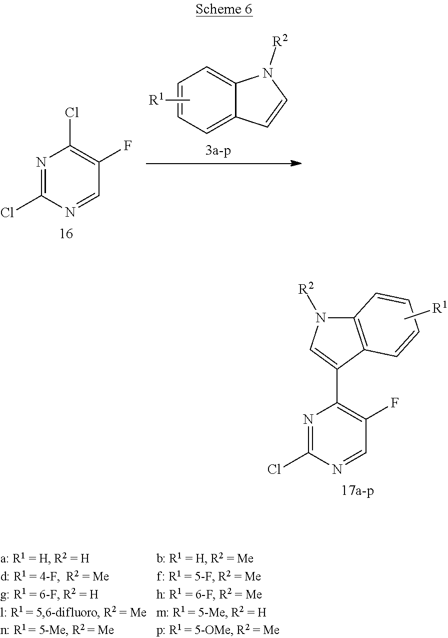

### Intermediate 17a: 3-(2-chloro-5-fluoropyrimidin-4-yl)-1H-indole (17a)

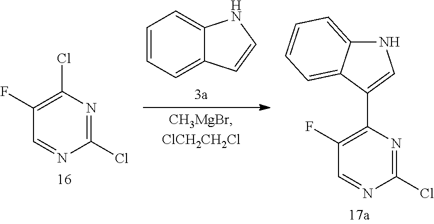

A solution of 3a (1.00 g, 8.54 mmol) in 1,2-dichloroethane (5 mL) was stirred at 0° C. for 10 min. Then MeMgBr was added dropwise followed by addition of the solution of 2,4-dichloro-5-fluoropyrimidine 16 (1.62 g, 9.70 mmol) in 1,2-dichloroethane (15 mL). The mixture was stirred at room temperature overnight. Then the reaction was quenched by H₂O (10 mL) and filtered off. The filter cake was dried in an air-circulating oven at 40° C. for 2.5 h to give the product 17a (0.6 g, yield 28.38%) as a brown solid.

### Intermediate 17b: 3-(2-chloro-5-fluoropyrimidin-4-yl)-1-methyl-1H-indole (17b)

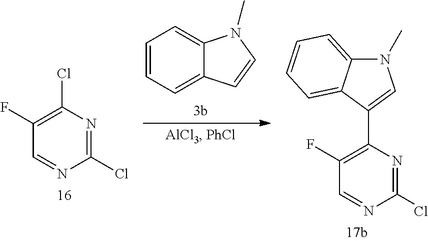

To a solution of 3b (2.08 g, 15.86 mmol) in chlorobenzene (50 mL), AlCl₃ (2.21 g, 16.57 mmol) and 2,4-dichloro-5-fluoropyrimidine 16 (2.72 g, 16.29 mmol) were added. The mixture was stirred at 70° C. for 5 h. The mixture was quenched by H₂O (20 mL) and was filtered off. The filter cake was washed with ethyl acetate (30 mL*2), and dried in an air-circulating oven at 40° C. overnight to give the product 17b (2.78 g, yield 67.0%) as a light brown solid.

### Intermediate 17d: 3-(2-chloro-5-fluoropyrimidin-4-yl)-4-fluoro-1-methyl-1H-indole (17d)

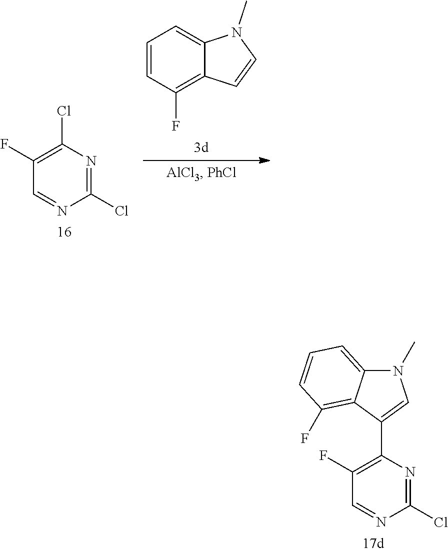 [SMILES: Fc1cnc(Cl)nc1Cl ; Cn1ccc2c(F)cccc21 ; Cn1cc(-c2nc(Cl)ncc2F)c2c(F)cccc21]

Synthesis of 17d was similar to that of 17b. The reaction of 3d (1.12 g, 7.51 mmol) and 2,4-dichloro-5-fluoropyrimidine 16 (1.04 g, 6.23 mmol) obtained the product 17d (0.76 g, yield 43.43%) as a brown solid.

### Intermediate 17f: 3-(2-chloro-5-fluoropyrimidin-4-yl)-5-fluoro-1-methyl-1H-indole (17f)

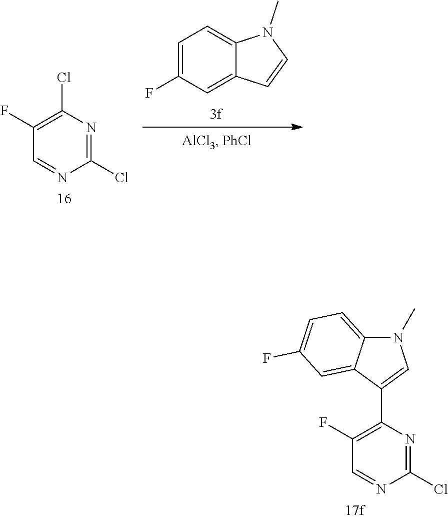 [SMILES: Fc1cnc(Cl)nc1Cl ; Cn1ccc2cc(F)ccc21 ; Cn1cc(-c2nc(Cl)ncc2F)c2cc(F)ccc21]

Synthesis of 17f was similar to that of 17b. The reaction of 3f (1.12 g, 7.51 mmol) and 2,4-dichloro-5-fluoropyrimidine 16 (1.01 g, 6.05 mmol) obtained the product 17f (0.98 g, yield 57.87%) as a gray solid.

### Intermediate 17g: 3-(2-chloro-5-fluoropyrimidin-4-yl)-6-fluoro-1H-indole (17g)

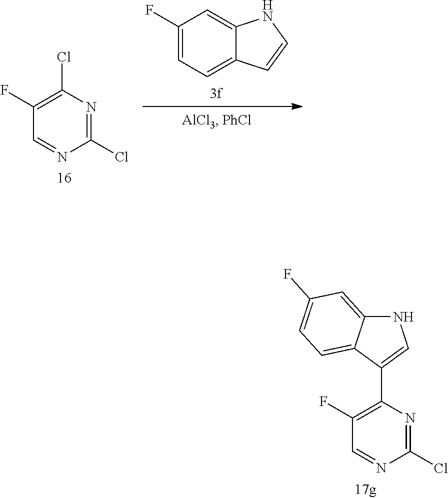 [SMILES: Fc1cnc(Cl)nc1Cl ; Fc1ccc2cc[nH]c2c1 ; Fc1ccc2c(-c3nc(Cl)ncc3F)c[nH]c2c1]

Synthesis of 17g was similar to that of 17b. The reaction of 3g (1.02 g, 7.55 mmol) and 2,4-dichloro-5-fluoropyrimidine 16 (1.33 g, 7.97 mmol) obtained the product 17g (0.87 g, yield 43.39%) as a light brown solid.

### Intermediate 17h: 3-(2-chloro-5-fluoropyrimidin-4-yl)-6-fluoro-1-methy-1H-indole (17h)

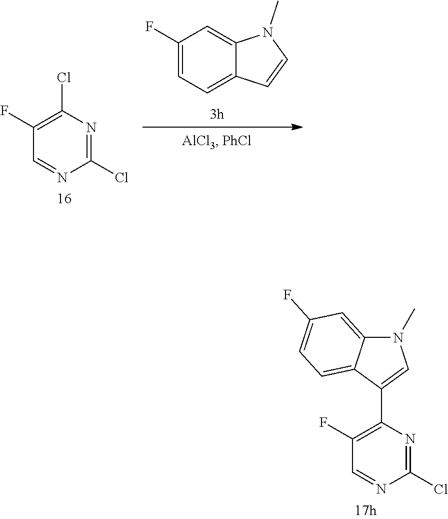 [SMILES: Fc1cnc(Cl)nc1Cl ; Cn1ccc2ccc(F)cc21 ; Cn1cc(-c2nc(Cl)ncc2F)c2ccc(F)cc21]

Synthesis of 17h was similar to that of 17b. The reaction of 3h (1.12 g, 7.51 mmol) and 2,4-dichloro-5-fluoropyrimidine 16 (1.02 g, 6.11 mmol) obtained the product 17h (0.97 g, yield 56.82%) as a light brown solid.

### Intermediate 171: 3-(2-chloro-5-fluoropyrimidin-4-yl)-5,6-difluoro-1-methyl-1H-indole (171)

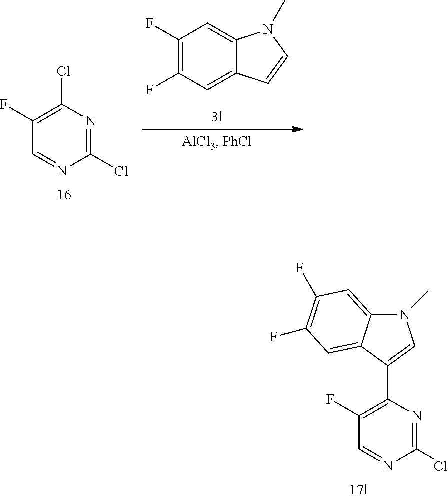 [SMILES: Fc1cnc(Cl)nc1Cl ; Cn1ccc2cc(F)c(F)cc21 ; Cn1cc(-c2nc(Cl)ncc2F)c2cc(F)c(F)cc21]

Synthesis of 171 was similar to that of 17b. The reaction of 31 (1.20 g, 7.18 mmol) and 2,4-dichloro-5-fluoropyrimidine 16 (1.20 g, 7.18 mmol) obtained the product 171 (1.31 g, yield 61.30%) as an off-white solid.

### Intermediate 17m: 3-(2-chloro-5-fluoropyrimidin-4-yl)-5-methyl-1H-indole (17m)

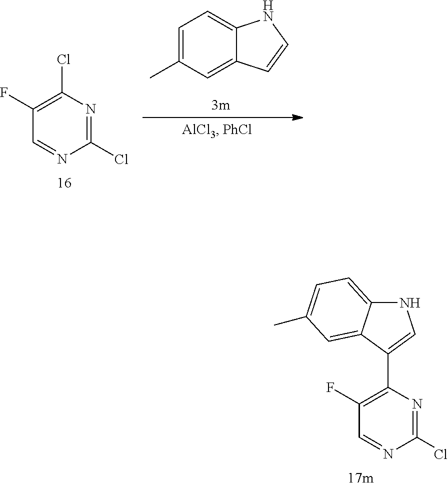

Synthesis of 17m was similar to that of 17b. The reaction of 3m (1.00 g, 7.62 mmol) and 2,4-dichloro-5-fluoropyrimidine 16 (1.30 g, 7.79 mmol) obtained the product 17m (0.78 g, yield 39.10%) as a gray solid.

### Intermediate 17n: 3-(2-chloro-5-fluoropyrimidin-4-yl)-1,5-dimethyl-1H-indole (17n)

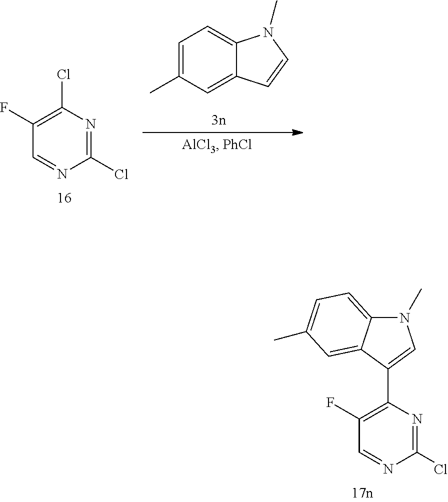 [SMILES: Fc1cnc(Cl)nc1Cl ; Cc1ccc2c(ccn2C)c1 ; Cc1ccc2c(c1)c(-c1nc(Cl)ncc1F)cn2C]

Synthesis of 17n was similar to that of 17b. The reaction of 3n (1.11 g, 7.64 mmol) and 2,4-dichloro-5-fluoropyrimidine 16 (1.08 g, 6.47 mmol) obtained the product 17n (0.75 g, yield 41.99%) as a gray solid.

### Intermediate 17p: 3-(2-chloro-5-fluoropyrimidin-4-yl)-5-methoxy-1-methyl-1H-indole (17p)

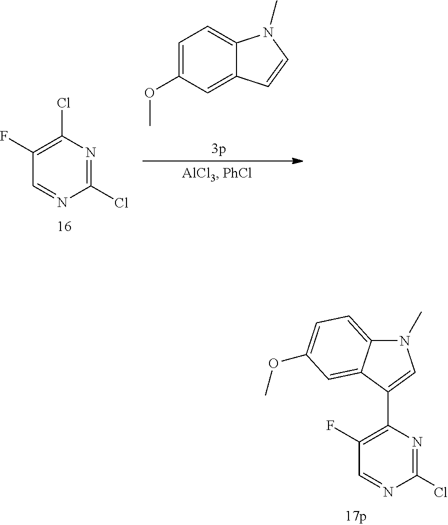 [SMILES: Fc1cnc(Cl)nc1Cl ; COc1ccc2c(ccn2C)c1 ; COc1ccc2c(c1)c(-c1nc(Cl)ncc1F)cn2C]

Synthesis of 17p was similar to that of 17b. The reaction of 3p (1.22 g, 7.57 mmol) and 2,4-dichloro-5-fluoropyrimidine 16 (1.26 g, 7.55 mmol) obtained the product 17p (0.73 g, yield 33.07%) as a gray solid.

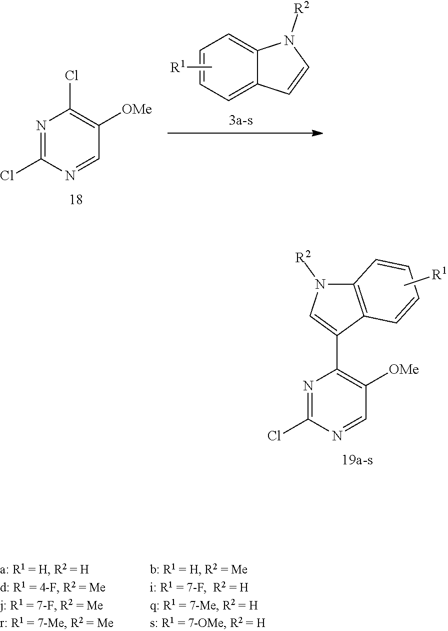

### Intermediate 19a: 3-(2-chloro-5-methoxypyrimidin-4-yl)-1H-indole (19a)

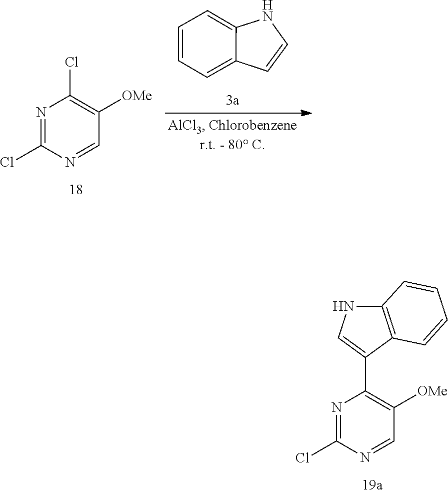 [SMILES: COc1cnc(Cl)nc1Cl ; c1ccc2[nH]ccc2c1 ; COc1cnc(Cl)nc1-c1c[nH]c2ccccc12]

AlCl₃ (5.01 g, 38 mmol) was added into a solution of 18 (5.35 g, 30 mmol) in PhCl (30 mL) at 20° C. within 5 min followed by stirring for 0.5 h. A solution of 3a (3.5 g, 30 mmol) in PhCl (30 mL) was then added to the reaction. The reaction mixture was stirred at 80° C. for 4 h. The mixture was quenched with water, extracted with dichloromethane. The combined organic phase was concentrated, purified by flash column chromatography (dichloromethane/methanol=100/1-30/1) to give the title compound 19a (1.8 g, yield 23%) as a brown solid. MS-ESI (M+H)⁺: 260.3, 262.4.

### Intermediate 19b: 3-(2-chloro-5-methoxypyrimidin-4-yl)-1-methyl-1H-indole (19b)

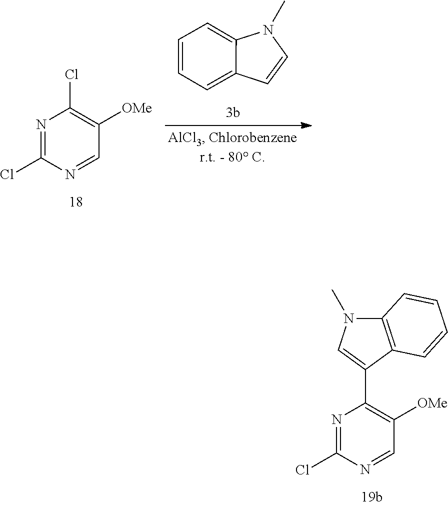

AlCl₃ (2.70 g, 20 mmol) was added into a solution of 18 (3.55 g, 20 mmol) in PhCl (20 mL) at 20° C. within 5 min followed by stirring for 0.5 h at 20° C. A solution of 3b (2.63 g, 20 mmol) in PhCl (15 mL) was added to the reaction. The reaction mixture was stirred at 80° C. for 4 h. The mixture was quenched with water, extracted with dichloromethane. The combined organic phase was concentrated, purified by flash column chromatography (petroleum ether/ethyl acetate=10/1-3/1) to give the title compound 19b (4.10 g, yield 75%) as a pink solid. MS-ESI (M+H)⁺: 308.2, 310.3.

### Intermediate 19d: 3-(2-chloro-5-methoxypyrimidin-4-yl)-4-fluoro-1-methyl-1H-indole (19d)

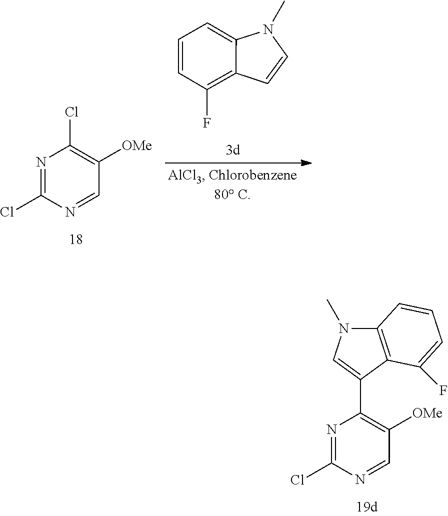 [SMILES: COc1cnc(Cl)nc1Cl ; Cn1ccc2c(F)cccc21 ; COc1cnc(Cl)nc1-c1cn(C)c2cccc(F)c12]

AlCl₃ (504.2 mg, 3.78 mmol) was added to a solution of 18 (607.0 mg, 3.39 mmol) in chlorobenzene (10 ml) at room temperature. The resulting solution was stirred for a while and then 4-fluoro-1-methyl-1H-indole 3d (0.6 g, 4.02 mmol) was added dropwise to the reaction mixture at room temperature. The resulting mixture was heated at 80° C. for 18 h. Then the reaction mixture was allowed to cool down and pour into ice-water. The mixture was stirred for another 2 h followed by addition of ethyl acetate. The mixture was filtered to afford part of the title compound 19d (489 mg). The filtrate was extracted with ethyl acetate. The organic layers were combined and concentrated under reduced pressure. The residue was purified by flash column chromatography (dichloromethane/methanol=10/1) to give another part of 4d (420 mg, totally 909 mg, yield 91.9%) as a gray solid

### Intermediate 19i: 3-(2-chloro-5-methoxypyrimidin-4-yl)-7-fluoro-1H-indole (19i)

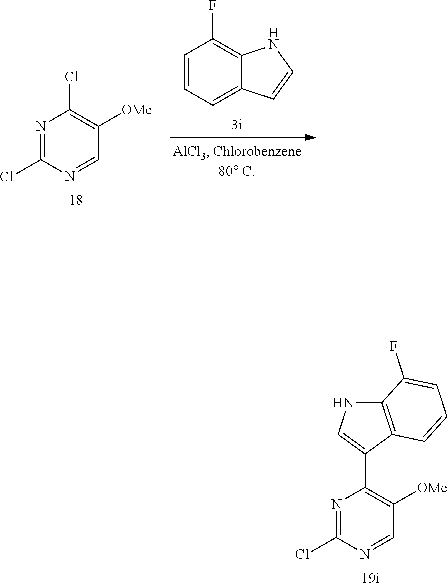 [SMILES: COc1cnc(Cl)nc1Cl ; Fc1cccc2cc[nH]c12 ; COc1cnc(Cl)nc1-c1c[nH]c2c(F)cccc12]

A mixture of 18 (400 mg, 2.24 mmol) and AlCl₃ (300 mg, 2.24 mmol) in chlorobenzene (20 mL) was stirred at room temperature for 30 min, then 3i (300 mg, 2.0 mmol) was added. The mixture was stirred at 80° C. for 3 h. The reaction was cooled to room temperature and quenched with H₂O (10 mL). The solid was collected by filtration, and washed by stirring in H₂O (5 mL) and ethyl acetate (5 mL) to give 19i as a pale yellow solid (400 mg, ˜72% yield).

### Intermediate 19j: 3-(2-chloro-5-methoxypyrimidin-4-yl)-7-fluoro-1-methyl-1H-indole (19i)

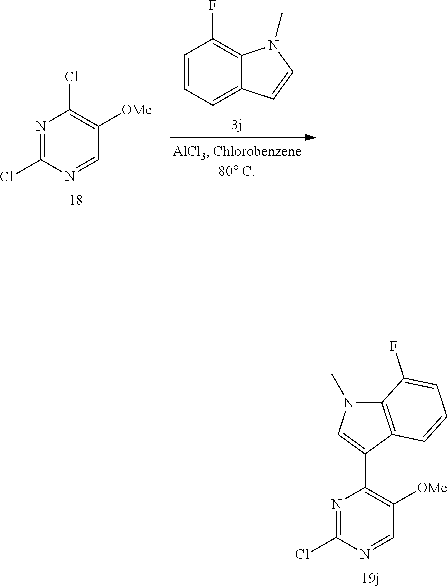 [SMILES: COc1cnc(Cl)nc1Cl ; Cn1ccc2cccc(F)c21 ; COc1cnc(Cl)nc1-c1cn(C)c2c(F)cccc12]

A mixture of 18 (2.5 g, 14 mmol) and AlCl₃ (2 g, 15 mmol) in chlorobenzene (100 mL) was stirred at room temperature for 30 min, then 3j (2.2 g, 14.8 mmol) was added. The mixture was stirred at 80° C. for 3 h. The reaction was cooled to 0° C. H₂O (6 mL) and HCl (1N, 3 mL) were added successively. The solid was collected by filtration, and washed by stirring in H₂O (10 mL) and ethyl acetate (10 mL) to give 19j as a purple solid (1.8 g, ˜44% yield). MS-ESI (M+H)⁺: 292.5, 294.6.

### Intermediate 19q: 3-(2-chloro-5-methoxypyrimidin-4-yl)-7-methyl-1H-indole (19q)

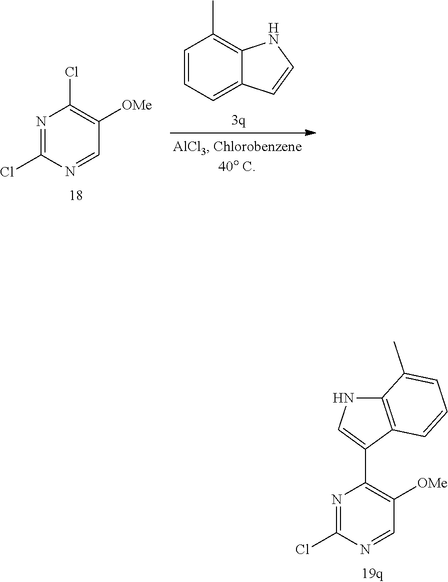

To a solution of 3q (0.50 g, 3.81 mmol) in chlorobenzene (10 mL), AlCl₃ (0.50 g, 3.75 mmol) and 2,4-dichloro-5-methoxypyrimidine 18 (0.68 g, 3.81 mmol) were added. The reaction mixture was stirred at 40° C. for 5 h followed by quenching with H₂O. The resulting mixture was stirred at room temperature for 10 min, and the precipitation was filtered off. The filter cake was washed with dichloromethane (5 mL*2), and dried in an air-circulating oven at 40° C. for 1.5 h to give the product 19q (1.02 g, yield 97.76%) as a gray solid.

### Intermediate 19r: 3-(2-chloro-5-methoxypyrimidin-4-yl)-1,7-dimethyl-1H-indole (19r)

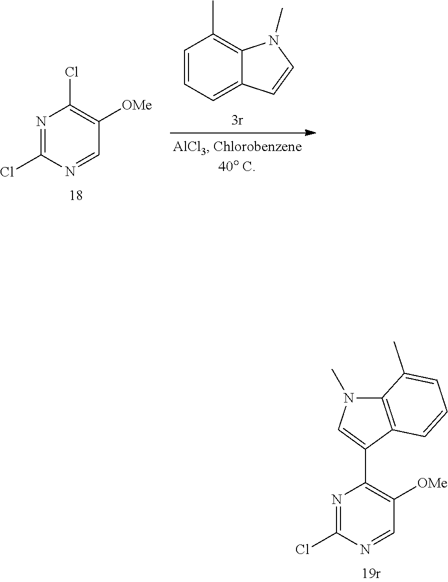 [SMILES: COc1cnc(Cl)nc1Cl ; Cc1cccc2ccn(C)c12 ; COc1cnc(Cl)nc1-c1cn(C)c2c(C)cccc12]

Synthesis of 19r was similar to that of 19q by using 3r (1.50 g, 10.33 mmol) and 2,4-dichloro-5-methoxypyrimidine 18 (1.36 g, 7.60 mmol) to give the product 19r (2.62 g, yield 119.87%) as a yellow solid.

### Intermediate 19s: 3-(2-chloro-5-methoxypyrimidin-4-yl)-7-methoxy-1H-indole (19s)

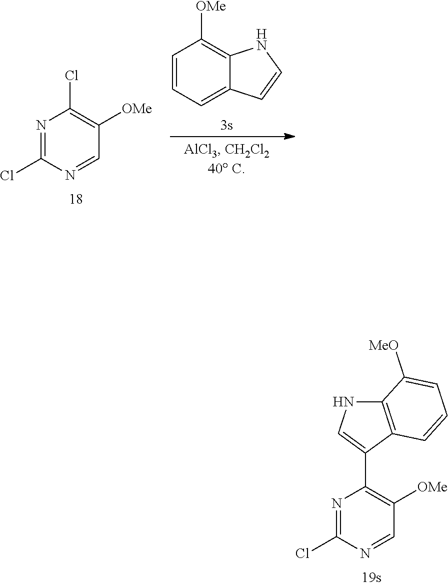 [SMILES: COc1cnc(Cl)nc1Cl ; COc1cccc2cc[nH]c12 ; COc1cnc(Cl)nc1-c1c[nH]c2c(OC)cccc12]

A mixture of 2,4-dichloro-5-methoxypyrimidine 18 (976 mg, 5.45 mmol) and AlCl₃ (718 mg, 5.45 mmol) in dichloromethane (30 mL) was stirred at room temperature for 30 min, then 7-methoxy-1-methyl-1H-indole 3s (897 mg, 5.56 mmol) was added. The mixture was stirred at 40° C. for 5 h. The reaction was cooled to room temperature. H₂O (10 mL) was added successively. The solid was collected by filtration, and washed by stirred in H₂O (5 mL) and ethyl acetate (5 mL) to give 19s as a black solid (1.2 g, ˜76% yield).

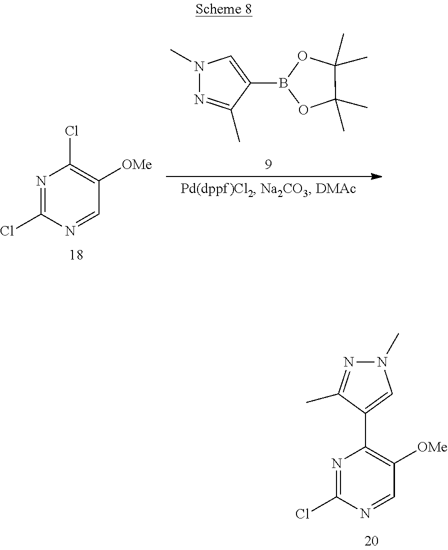

### Intermediate 20: 2-chloro-4-(1,3-dimethyl-1H-pyrazol-4-yl)-5-methoxypyrimidine (20)

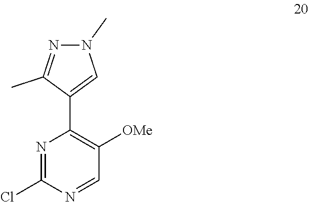 [SMILES: COc1cnc(Cl)nc1-c1cn(C)nc1C]

A mixture of 2,4-dichloro-5-methoxypyrimidine 18 (1.0 g, 5.6 mmol), 1,3-Dimethyl-1H-pyrazole-4-boronic acid pinacol ester 9 (1.22 g, 5.5 mmol), Pd(dppf)Cl₂ (0.2 g, 0.27 mmol) and Na₂CO₃ (2M a.q., 10 ml, 20 mmol) in N,N-dimethylacetamide (20 mL) was stirred under N₂ at 80° C. for 1 h. After cooling to room temperature, the reaction mixtures was filtered through Celite® and the filtrates were extracted with ethyl acetate. The organic layer was separated, washed with water and concentrated under reduced pressure to dryness. The crude was purified by flash column chromatography (eluting with ethyl acetate) to give the title compound 20 (0.8 g, yield 61.0%) as a white solid.

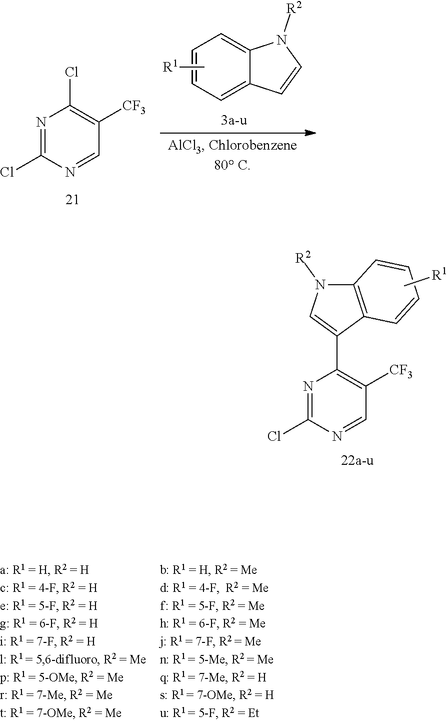

### Intermediate 22b: 3-(2-chloro-5-(trifluoromethyl)pyrimidin-4-yl)-1-methyl-1H-indole (22b)

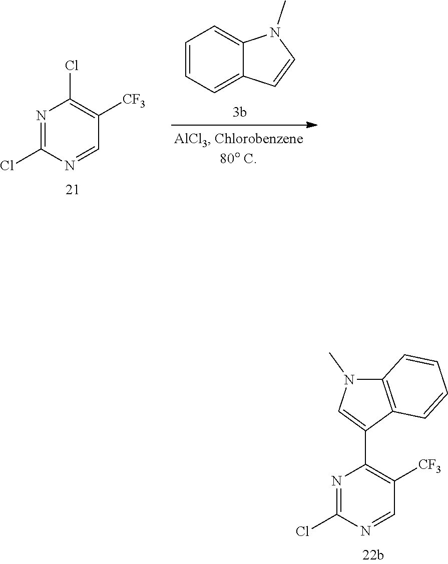

To a solution of 3b (1.01 g, 7.70 mmol) in chlorobenzene (15 mL), AlCl₃ (0.92 g, 6.90 mmol) and 2,4-dichloro-5-(trifluoromethyl)pyrimidine 21 (1.52 g, 7.01 mmol) were added. The mixture was stirred at 80° C. for 2 h. The mixture was quenched by H₂O (20 mL) and was filtered off. The filter cake was washed with ethyl acetate (20 mL*2), and dried in an air-circulating oven at 40° C. overnight to give the product 22b (0.79 g, yield 35.9%) as a brown solid.

### Intermediate 22a: 3-(2-chloro-5-(trifluoromethyl)pyrimidin-4-yl)-1H-indole (22a)

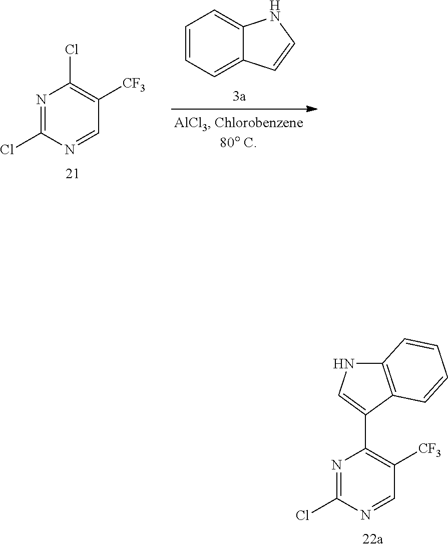 [SMILES: FC(F)(F)c1cnc(Cl)nc1Cl ; c1ccc2[nH]ccc2c1 ; FC(F)(F)c1cnc(Cl)nc1-c1c[nH]c2ccccc12]

Synthesis of 22a was similar to that of 22b by using 3a (1.02 g, 8.71 mmol) and 2,4-dichloro-5-(trifluoromethyl)pyrimidine 21 (1.56 g, 7.19 mmol) to afford the product 22a (1.33 g, yield 62.09%) as a gray solid.

### Intermediate 22c: 3-(2-chloro-5-(trifluoromethyl)pyrimidin-4-yl)-4-fluoro-1H-indole (22c)

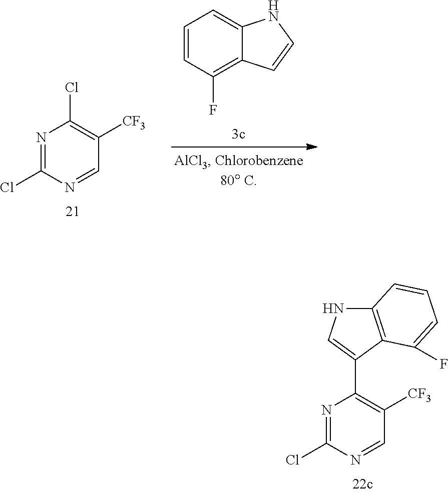 [SMILES: FC(F)(F)c1cnc(Cl)nc1Cl ; Fc1cccc2[nH]ccc12 ; Fc1cccc2[nH]cc(-c3nc(Cl)ncc3C(F)(F)F)c12]

Synthesis of 22c was similar to that of 22b by using 3c (2.02 g, 14.95 mmol) and 2,4-dichloro-5-(trifluoromethyl)pyrimidine 21 (2.94 g, 13.55 mmol) to afford the product 22c (0.67 g, yield 15.67%) as a gray solid.

### Intermediate 22d: 3-(2-chloro-5-(trifluoromethyl)pyrimidin-4-yl)-4-fluoro-1-methyl-1H-indole (22d)

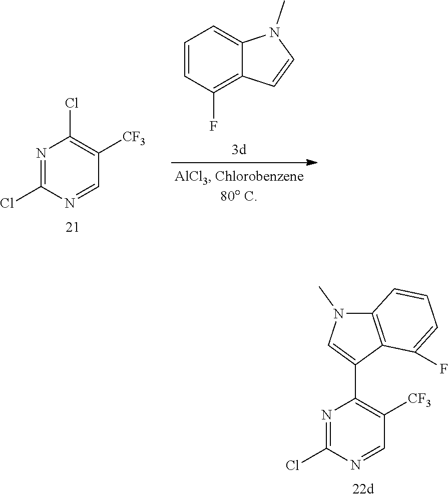 [SMILES: FC(F)(F)c1cnc(Cl)nc1Cl ; Cn1ccc2c(F)cccc21 ; Cn1cc(-c2nc(Cl)ncc2C(F)(F)F)c2c(F)cccc21]

Synthesis of 22d was similar to that of 22b by using 3d (1.11 g, 7.44 mmol) and 2,4-dichloro-5-(trifluoromethyl)pyrimidine 21 (1.30 g, 5.99 mmol) to afford the product 22d (0.36 g, yield 18.20%) as a dark brown solid.

### Intermediate 22e: 3-(2-chloro-5-(trifluoromethyl)pyrimidin-4-yl)-5-fluoro-1H-indole (22e)

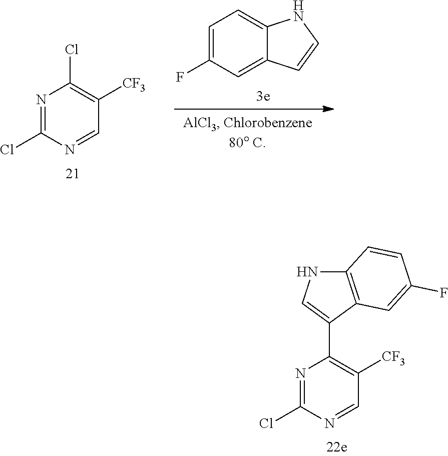 [SMILES: FC(F)(F)c1cnc(Cl)nc1Cl ; Fc1ccc2[nH]ccc2c1 ; Fc1ccc2[nH]cc(-c3nc(Cl)ncc3C(F)(F)F)c2c1]

Synthesis of 22e was similar to that of 22b by using 3e (2.01 g, 14.87 mmol) and 2,4-dichloro-5-(trifluoromethyl)pyrimidine 21 (2.95 g, 13.60 mmol) to afford the product 22e (2.42 g, yield 56.18%) as a gray solid.

### Intermediate 22f: 3-(2-chloro-5-(trifluoromethyl)pyrimidin-4-yl)-5-fluoro-1-methyl-1H-indole (22f)

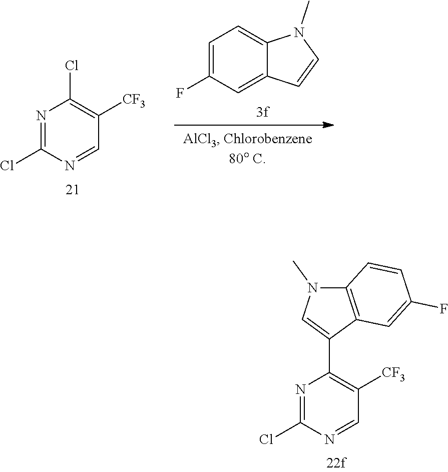 [SMILES: FC(F)(F)c1cnc(Cl)nc1Cl ; Cn1ccc2cc(F)ccc21 ; Cn1cc(-c2nc(Cl)ncc2C(F)(F)F)c2cc(F)ccc21]

Synthesis of 22f was similar to that of 22b by using 3f (1.01 g, 6.77 mmol) and 2,4-dichloro-5-(trifluoromethyl)pyrimidine 21 (1.21 g, 5.58 mmol) to afford the product 22f (0.78 g, yield 42.28%) as a gray solid.

### Intermediate 22g: 3-(2-chloro-5-(trifluoromethyl)pyrimidin-4-yl)-6-fluoro-1H-indole (22g)

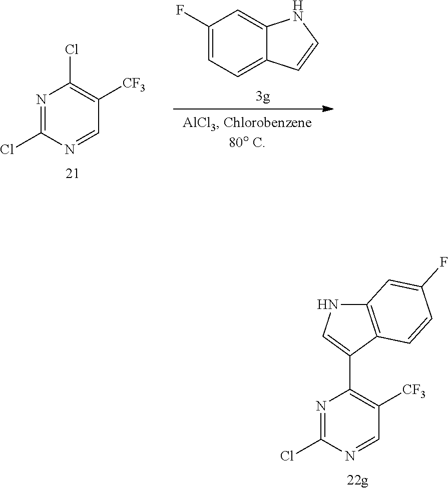 [SMILES: FC(F)(F)c1cnc(Cl)nc1Cl ; Fc1ccc2cc[nH]c2c1 ; Fc1ccc2c(-c3nc(Cl)ncc3C(F)(F)F)c[nH]c2c1]

Synthesis of 22g was similar to that of 22b by using 3g (2.10 g, 15.54 mmol) and 2,4-dichloro-5-(trifluoromethyl)pyrimidine 21 (3.37 g, 15.54 mmol) to afford the product 22g (1.60 g, yield 32.62%) as a gray solid.

### Intermediate 22h: 3-(2-chloro-5-(trifluoromethyl)pyrimidin-4-yl)-6-fluoro-1-methyl-1H-indole (22h)

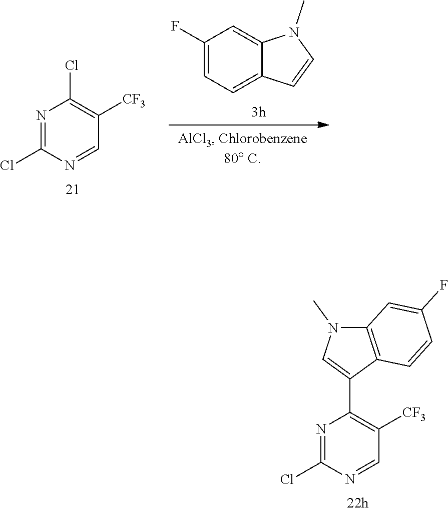 [SMILES: FC(F)(F)c1cnc(Cl)nc1Cl ; Cn1ccc2ccc(F)cc21 ; Cn1cc(-c2nc(Cl)ncc2C(F)(F)F)c2ccc(F)cc21]

Synthesis of 22h was similar to that of 22b by using 3h (1.02 g, 6.84 mmol) and 2,4-dichloro-5-(trifluoromethyl)pyrimidine 21 (1.21 g, 5.58 mmol) to afford the product 22h (0.53 g, yield 28.92%) as a light brown solid.

### Intermediate 22i: 3-(2-chloro-5-(trifluoromethyl)pyrimidin-4-yl)-7-fluoro-1H-indole (22i)

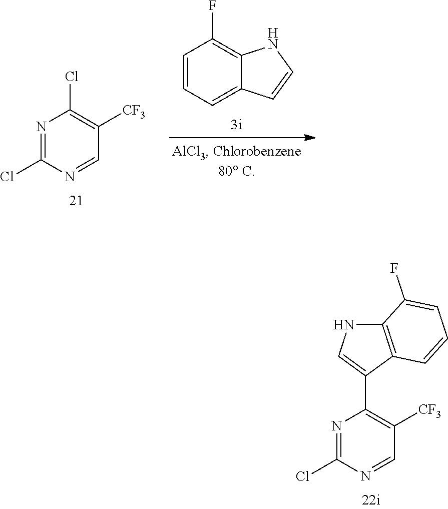 [SMILES: FC(F)(F)c1cnc(Cl)nc1Cl ; Fc1cccc2cc[nH]c12 ; Fc1cccc2c(-c3nc(Cl)ncc3C(F)(F)F)c[nH]c12]

Synthesis of 22i was similar to that of 22b by using 3i (5.00 g, 37.00 mmol) and 2,4-dichloro-5-(trifluoromethyl)pyrimidine 21 (7.99 g, 36.82 mmol) to afford the product 22i (6.23 g, yield 53.34%) as a purple solid.

### Intermediate 22j: 3-(2-chloro-5-(trifluoromethyl)pyrimidin-4-yl)-7-fluoro-1-methyl-1H-indole (22j)

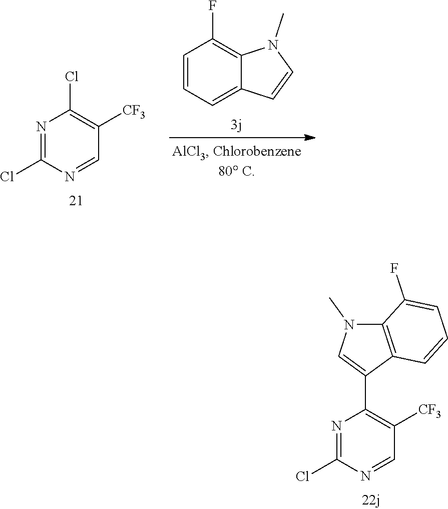 [SMILES: FC(F)(F)c1cnc(Cl)nc1Cl ; Cn1ccc2cccc(F)c21 ; Cn1cc(-c2nc(Cl)ncc2C(F)(F)F)c2cccc(F)c21]

Synthesis of 22j was similar to that of 22b by using 3j (2.21 g, 14.82 mmol) and 2,4-dichloro-5-(trifluoromethyl)pyrimidine 21 (3.24 g, 14.93 mmol) to afford the product 22j (1.42 g, yield 29.07%) as a light brown solid.

### Intermediate 221: 3-(2-chloro-5-(trifluoromethyl)pyrimidin-4-yl)-5,6-difluoro-1-methyl-1H-indole (221)

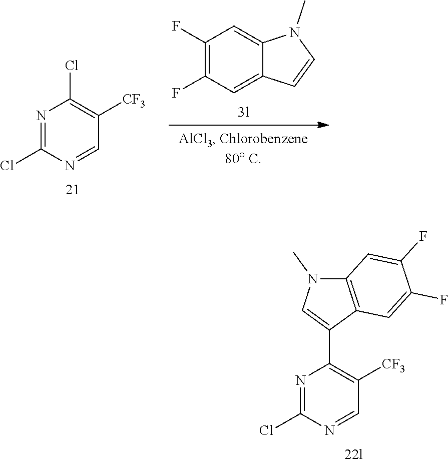 [SMILES: FC(F)(F)c1cnc(Cl)nc1Cl ; Cn1ccc2cc(F)c(F)cc21 ; Cn1cc(-c2nc(Cl)ncc2C(F)(F)F)c2cc(F)c(F)cc21]

Synthesis of 221 was similar to that of 22b by using 31 (0.60 g, 3.59 mmol) and 2,4-dichloro-5-(trifluoromethyl)pyrimidine 21 (0.79 g, 3.64 mmol) to afford the product 221 (0.38 g, yield 30.45%) as a brown solid.

### Intermediate 22n: 3-(2-chloro-5-(trifluoromethyl)pyrimidin-4-yl)-1,5-dimethyl-1H-indole (22n)

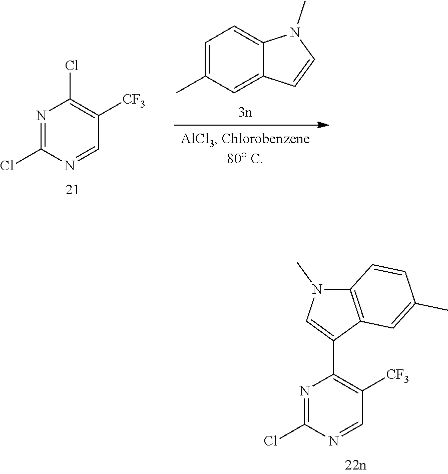 [SMILES: FC(F)(F)c1cnc(Cl)nc1Cl ; Cc1ccc2c(ccn2C)c1 ; Cc1ccc2c(c1)c(-c1nc(Cl)ncc1C(F)(F)F)cn2C]

Synthesis of 22n was similar to that of 22b by using 3n (0.90 g, 6.20 mmol) and 2,4-dichloro-5-(trifluoromethyl)pyrimidine 21 (1.01 g, 4.65 mmol) to afford the product 22n (0.39 g, yield 25.69%) as a brown solid.

### Intermediate 22p: 3-(2-chloro-5-(trifluoromethyl)pyrimidin-4-yl)-5-methoxy-1-methyl-1H-indole (22p)

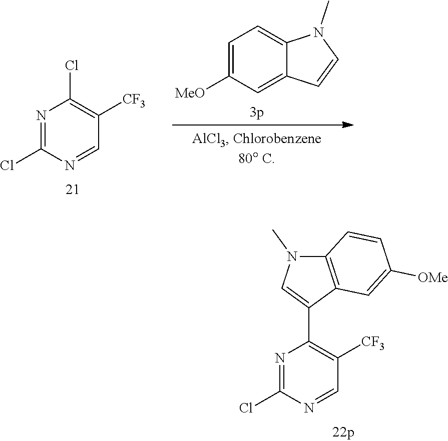 [SMILES: FC(F)(F)c1cnc(Cl)nc1Cl ; COc1ccc2c(ccn2C)c1 ; COc1ccc2c(c1)c(-c1nc(Cl)ncc1C(F)(F)F)cn2C]

Synthesis of 22p was similar to that of 22b by using 3p (1.12 g, 6.95 mmol) and 2,4-dichloro-5-(trifluoromethyl)pyrimidine 21 (1.22 g, 5.62 mmol) to afford the product 22p (0.24 g, yield 12.43%) as a brown solid.

### Intermediate 22q: 3-(2-chloro-5-(trifluoromethyl)pyrimidin-4-yl)-7-methyl-1H-indole (22q)

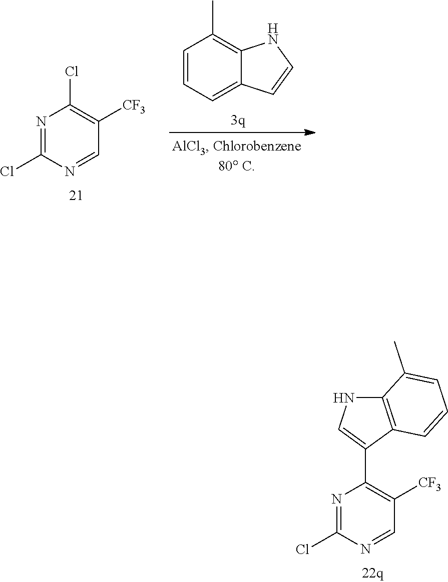

Synthesis of 22q was similar to that of 22b by using 3q (2.01 g, 15.32 mmol) and 2,4-dichloro-5-(trifluoromethyl)pyrimidine 21 (3.32 g, 15.32 mmol) to afford the product 22q (3.00 g, yield 62.81%) as a gray solid.

### Intermediate 22r: 3-(2-chloro-5-(trifluoromethyl)pyrimidin-4-yl)-7-methyl-1-methyl-1H-indole (22r)

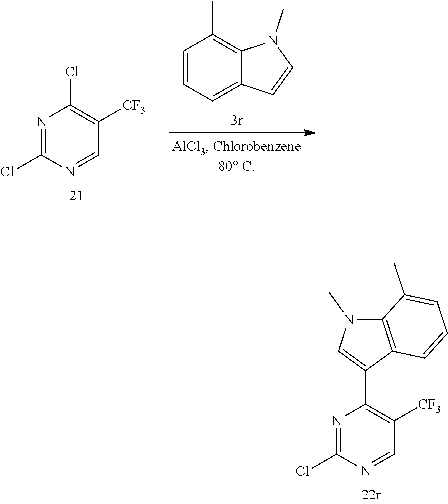 [SMILES: FC(F)(F)c1cnc(Cl)nc1Cl ; Cc1cccc2ccn(C)c12 ; Cc1cccc2c(-c3nc(Cl)ncc3C(F)(F)F)cn(C)c12]

Synthesis of 22r was similar to that of 22b by using 3r (5.53 g, 38.09 mmol) and 2,4-dichloro-5-(trifluoromethyl)pyrimidine 21 (8.26 g, 38.09 mmol) to afford the product 22r (7.44 g, yield 59.98%) as a purplish red solid.

### Intermediate 22s: 3-(2-chloro-5-(trifluoromethyl)pyrimidin-4-yl)-7-methoxy-1H-indole (22s)

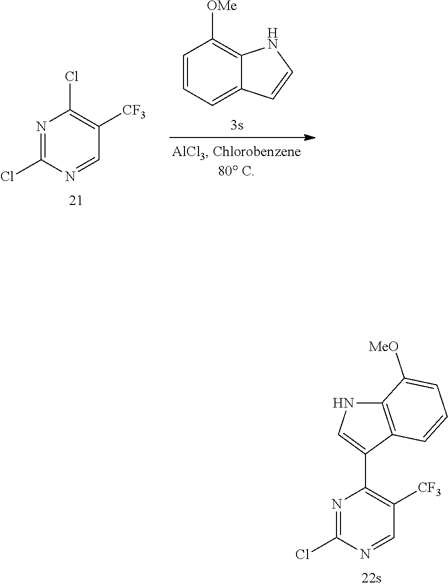 [SMILES: FC(F)(F)c1cnc(Cl)nc1Cl ; COc1cccc2cc[nH]c12 ; COc1cccc2c(-c3nc(Cl)ncc3C(F)(F)F)c[nH]c12]

Synthesis of 22s was similar to that of 22b by using 3s (1.97 g, 13.39 mmol) and 2,4-dichloro-5-(trifluoromethyl)pyrimidine (2.94 g, 13.55 mmol) to afford the product 22s (2.00 g, yield 45.60%) as a brown solid.

### Intermediate 22t: 3-(2-chloro-5-(trifluoromethyl)pyrimidin-4-yl)-7-methoxy-1-methyl-1H-indole (22t)

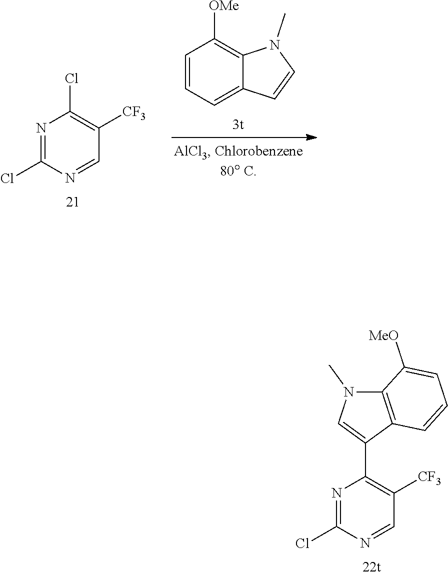 [SMILES: FC(F)(F)c1cnc(Cl)nc1Cl ; COc1cccc2ccn(C)c12 ; COc1cccc2c(-c3nc(Cl)ncc3C(F)(F)F)cn(C)c12]

Synthesis of 22t was similar to that of 22b by using 3t (2.19 g, 13.59 mmol) and 2,4-dichloro-5-(trifluoromethyl)pyrimidine (2.96 g, 13.64 mmol) to afford the product 22t (1.86 g, yield 40.07%) as a brown solid.

### Intermediate 22u: 3-(2-chloro-5-(trifluoromethyl)pyrimidin-4-yl)-1-ethyl-5-fluoro-1H-indole

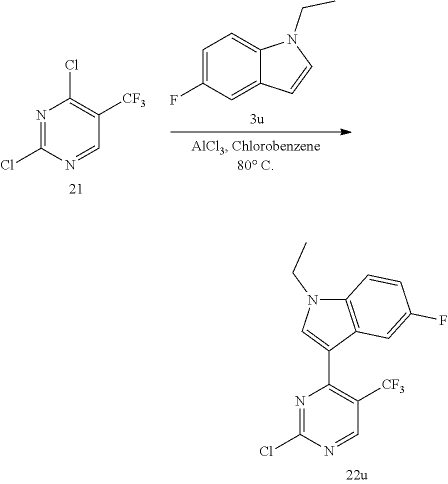

Synthesis of 22u was similar to that of 22b by using 3u (675 mg, 4.14 mmol) and 2,4-dichloro-5-(trifluoromethyl)pyrimidine (703 mg, 3.24 mmol) to afford the product 22u (601 mg, yield 53.9%) as a red solid.

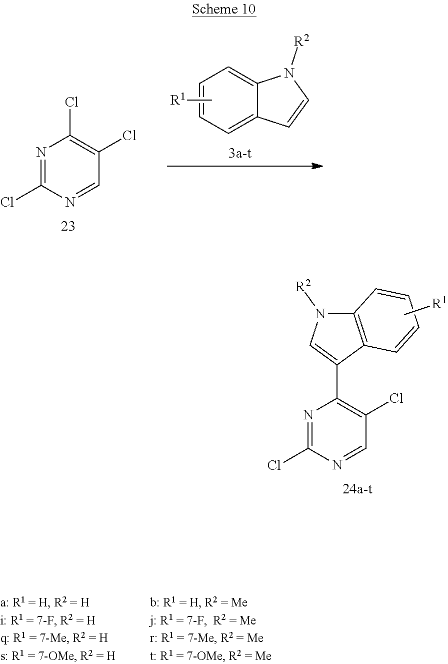

### Intermediate 24a: 3-(2, 5-dichloropyrimidin-4-yl)-1H-indole (24a)

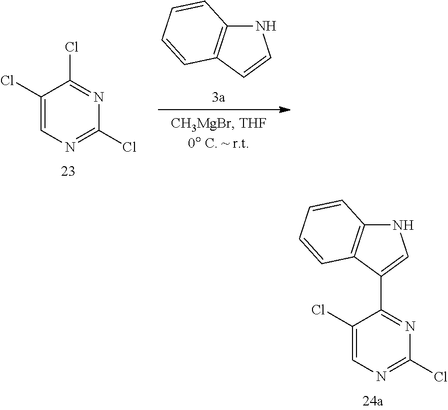 [SMILES: Clc1ncc(Cl)c(Cl)n1 ; c1ccc2[nH]ccc2c1 ; Clc1ncc(Cl)c(-c2c[nH]c3ccccc23)n1]

CH₃MgBr (3.0M in 2-methyltetrahydrofuran, 36.5 mL, 109 mmol) was added dropwise over 10 minutes to a solution of indole (12.8 g, 109 mmol) in THF (150 mL) at 0° C. The solution was then stirred at 0° C. for 0.5 h. 23 (10.0 g, 54.5 mmol) was then added, resulting in a yellow solution. The ice bath was removed, then the solution was stirred at room temperature for 1 h, resulting in a red solution. The mixture was heated to 60° C. and then stirred at 60° C. for 1.5 h. The mixture was then cooled to room temperature and sat.aq.NH₄Cl was added (300 mL). The mixture was extracted with ethyl acetate (300 mL×3). The organic layers were dried over Na₂SO₄, filtered and concentrated under reduced pressure to give a residue. The residue was purified by column (petroleum ether:ethyl acetate=15:1-5:1-1:100) to afford 24a (11 g, yield 76.4%) as a yellow solid. ¹H NMR (400 MHz, DMSO-d6) δ 12.26 (s, 1H), 8.74 (s, 1H), 8.72 (d, J=3.2 Hz, 1H), 8.52 (dd, J=6.6, 2.5 Hz, 1H), 7.55 (dd, J=6.4, 2.2 Hz, 1H), 7.30-7.23 (m, 2H).

### Intermediate 24b: 3-(2,5-dichloropyrimidin-4-yl)-1-methyl-1H-indole (24b)

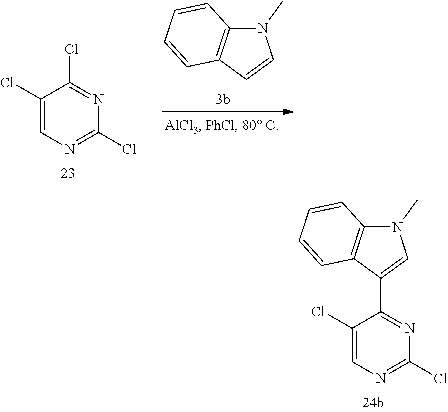

A mixture of compound 23 (1.0 g, 5.4 mmol) and AlCl₃ (1.45 g, 10.9 mmol) in PhCl (10 mL) was stirred at room temperature for 30 min, then 1-methyl-1H-indole 3b (1.07 g, 8.18 mmol) was added. The mixture was stirred at 80° C. for overnight. The reaction was cooled to 0° C. H₂O (3 mL) and HCl (1N, 1.5 mL) were added successively. The solid was collected by filtration. Crude product was slurried with H₂O (5 mL) and ethyl acetate (5 mL) to give 24b as a yellow solid (1.46 g, yield 96.7%). MS-ESI (M+H)⁺: 277.9. ¹H NMR (400 MHz, DMSO-d6) δ 8.72 (d, J=7.6 Hz, 1H), 8.42 (d, J=7.6 Hz, 2H), 7.38 (s, 3H), 3.91 (s, 3H).

### Intermediate 24i: 3-(2,5-dichloropyrimidin-4-yl)-7-fluoro-1H-indole (24i)

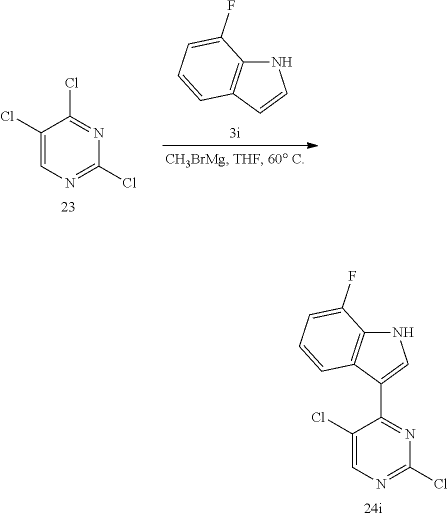 [SMILES: Clc1ncc(Cl)c(Cl)n1 ; Fc1cccc2cc[nH]c12 ; Fc1cccc2c(-c3nc(Cl)ncc3Cl)c[nH]c12]

CH₃BrMg in ethyl ether (3M, 6.1 ml, 18.8 mmol) was added to a stirred solution of 7-fluoro-1H-indole 3i (2.67 g, 19.78 mmol) in tetrahydrofuran (30 ml) cooled by ice-salt bath. The reaction mixture was stirred for 0.5 h. 2,4,6-trichloropyrimidine 23 (1.85 g, 10.01 mmol) was added dropwise to the reaction mixture followed by stirring for 0.5 h. The resulting mixture was heated to 60° C. and stirred for 3 h. Then the reaction mixture was cooled down and filtered. The filtrate was concentrated. The residue was stirred with ethyl acetate (20 ml) and petroleum ether (20 ml) for 0.5 h. Then the mixture was filtered and the filter cake was dried in vacuo to give the title compound 24i (1.8 g, yield 63.82%).

### Intermediate 24j: 3-(2,5-dichloropyrimidin-4-yl)-7-fluoro-1-methyl-1H-indole (24j)

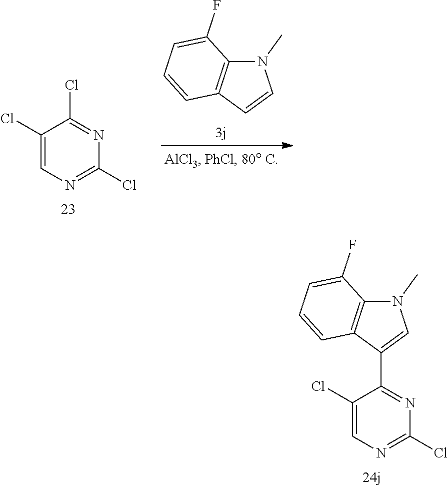 [SMILES: Clc1ncc(Cl)c(Cl)n1 ; Cn1ccc2cccc(F)c21 ; Cn1cc(-c2nc(Cl)ncc2Cl)c2cccc(F)c21]

AlCl₃ (1.52 g, 11.40 mmol) was added into a solution of 23 (1.02 g, 5.56 mmol) in PhCl (50 mL) at 20° C. Then the reaction was stirred for 0.5 h at 20° C. A solution of 3j (1.05 g, 6.41 mmol) in PhCl (5 mL) was added to the reaction. The reaction was stirred for 3 h at 80° C. Then the reaction was cooled to room temperature and quenched with 4 N HCl aq. (30 mL). The mixture was extracted with dichloromethane. The organic layer was washed with water, sat. NaHCO₃, brine and dried over Na₂SO₄. The organic layer was filtered and concentrated under reduced pressure to give a residue. The residue was purified by flash column chromatography (petroleum ether/ethyl acetate=2/1˜1/1) to afford 24j (1.19 g, yield 73.7%) as yellow solid. MS-ESI (M+H)⁺: 224.0.

### Intermediate 24q: 3-(2,5-dichloropyrimidin-4-yl)-7-methyl-1H-indole (24q)

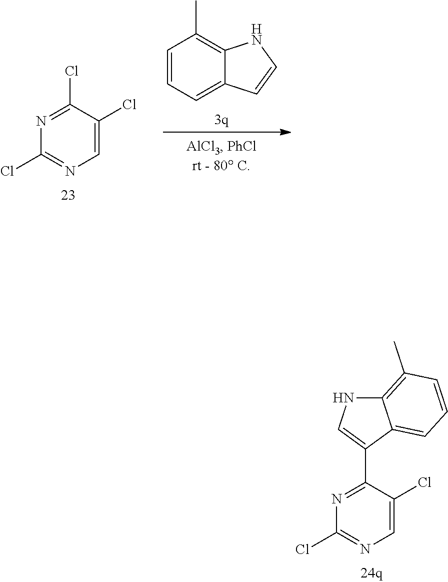

To a solution of 23 (2.06 g, 11.23 mmol) in PhCl (50 mL) was added AlCl₃ (2.24 g, 16.80 mmol) in batches and the reaction was stirred for 0.5 h at 20° C. Then a solution of 3q (1.73 g, 13.19 mmol) in PhCl (5 mL) was added. The resulting mixture was stirred at 80° C. for 5 h. The reaction was cooled to room temperature and quenched with 4 N HCl aq. (15 mL). The mixture was diluted with dichloromethane (100 mL), filtered through gel silica and concentrated to obtain crude product. The crude was slurried by ethyl acetate (20 mL) to afford 24q (1.81 g, yield 57.9%) as gray solid.

### Intermediate 24r: 3-(2,5-dichloropyrimidin-4-yl)-1,7-dimethyl-1H-indole (24r)

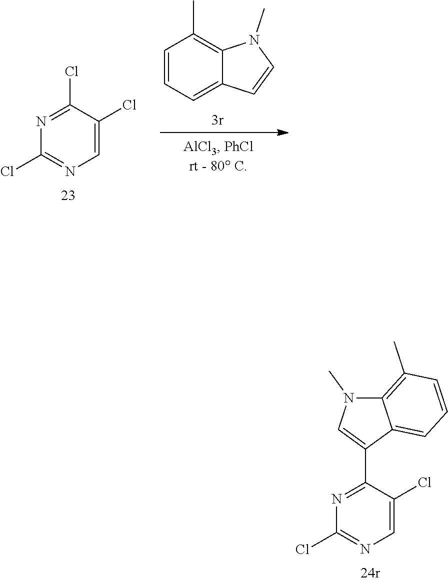

To a solution of 23 (3.56 g, 19.41 mmol) in PhCl (100 mL) was added AlCl₃ (3.81 g, 28.57 mmol) in batches and the reaction was stirred for 0.5 h at 20° C. Then a solution of 3r (3.34 g, 23.00 mmol) in PhCl (5 mL) was added. The resulting mixture was stirred at 80° C. for 3 h. The reaction was cooled to room temperature and quenched with 4 N HCl aq. (90 mL). The mixture was diluted with dichloromethane (100 mL), filtered through gel silica and concentrated to obtain crude product. The crude was slurried by ethyl acetate (20 mL) to afford 24r (5.01 g, yield 89.9%) as white solid.

### Intermediate 24s: 3-(2,5-dichloropyrimidin-4-yl)-7-methoxy-1H-indole (24s)

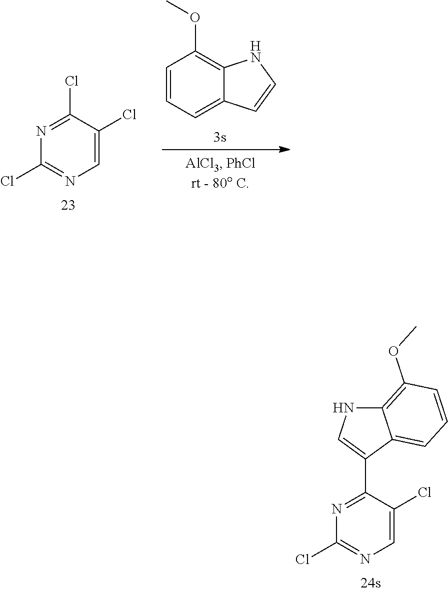

AlCl₃ (2.56 g, 19.2 mmol) was added into a solution of 2,4,5-trichloro pyrimidine 23 (2.71 g, 14.8 mmol) in PhCl (30 mL) at 20° C. within 3 min and then stirred for 0.5 h at 20° C. A solution of 7-methoxy-1H-indole 3s (2.22 g, 15.1 mmol) in PhCl (20 mL) was added to the reaction. The resulting mixture was stirred at 80° C. for 3 h. The mixture was quenched with water, extracted with dichloromethane. The combined organic phase was concentrated, purified by flash column chromatography (dichloromethane/methanol=100/1-30/1) to give the title compound 24s (2.1 g, 48% yield) as a brown solid. MS-ESI (M+H)⁺:294.2/296.3.

### Intermediate 24t: 3-(2,5-dichloropyrimidin-4-yl)-7-methoxy-1-methyl-1H-indole (24t)

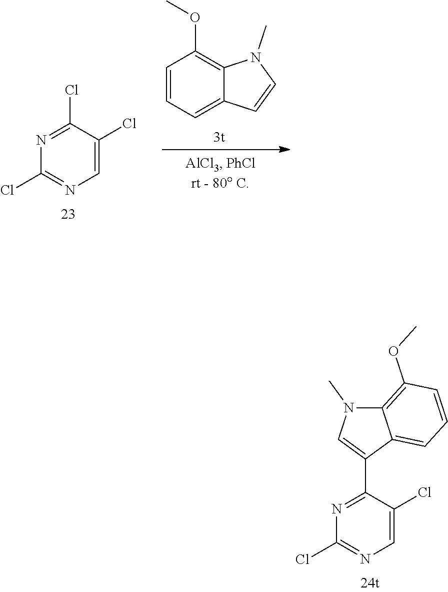

AlCl₃ (2.68 g, 20 mmol) was added into a solution of 2,4,5-trichloro pyrimidine 23 (3.67 g, 20 mmol) in PhCl (30 mL) at 20° C. within 5 min and then stirred for 0.5 h at 20° C. A solution of 7-methoxy-1-methyl-1H-indole 3t (3.26 g, 20 mmol) in PhCl (20 mL) was added to the reaction. The resulting mixture was stirred at 80° C. for 3 h. The mixture was quenched with water, extracted with dichloromethane. The combined organic phase was concentrated, purified by flash column chromatography (petroleum ether/ethyl acetate=10/1-3/1) to give the title compound 24t (4.5 g, 75% yield) as a pink solid. MS-ESI (M+H)⁺:308.2/310.3.

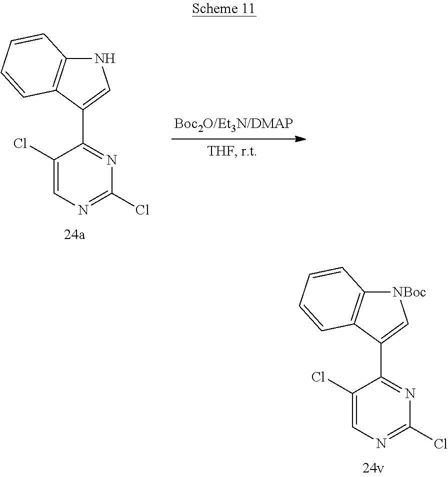 [SMILES: Clc1ncc(Cl)c(-c2c[nH]c3ccccc23)n1 ; CC(C)(C)OC(=O)n1cc(-c2nc(Cl)ncc2Cl)c2ccccc21]

### Intermediate 24v: tert-butyl 3-(2, 5-dichloropyrimidin-4-yl)-1H-indole-1-carboxylate (24v)

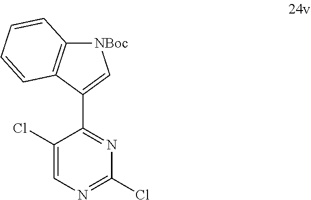 [SMILES: CC(C)(C)OC(=O)n1cc(-c2nc(Cl)ncc2Cl)c2ccccc21]

To a solution of 24a (5.0 g, 18.9 mmol) in 100 mL of THF was added Boc₂O (4.96 g, 22.7 mmol), Et₃N (3.2 mL, 22.7 mmol), DMAP (232 mg, 1.89 mmol). The mixture was stirred at room temperature under Ar overnight. The mixture was poured into water (200 mL), extracted with dichloromethane (300×3 mL). The organic layers were dried over Na₂SO₄, filtered and concentrated under reduced pressure to give a residue. The residue was purified by column (petroleum ether/ethyl acetate=100/1˜10/1˜1/5) to afford 24v (5.0 g, yield 72.5%) as a yellow solid. ¹H NMR (400 MHz, CDCl₃) δ 8.81 (s, 1H), 8.61 (s, 1H), 8.56-8.53 (m, 1H), 8.20 (dd, J=6.8, 2.1 Hz, 1H), 7.46-7.38 (m, 2H), 1.73 (s, 9H).

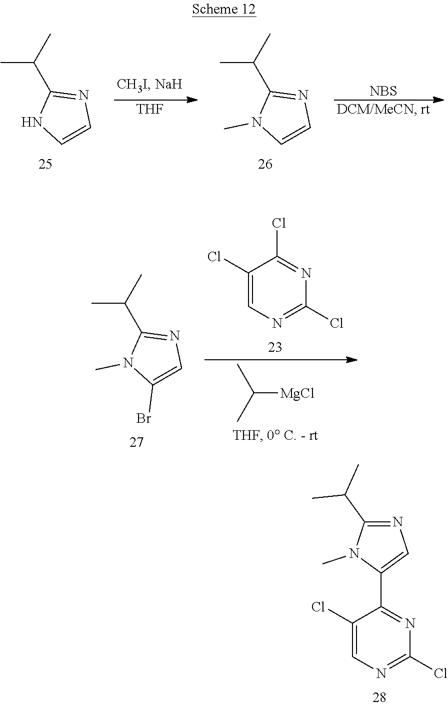

### Intermediate 28: 2-isopropyl-1-methyl-1H-imidazole (26)

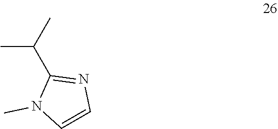

To the mixture of NaH (5.45 g, 136.17 mmol) in anhydrous THF (100 mL) was added 25 (10 g, 90.78 mmol) in batches at 0° C. The mixture was stirred at 0° C. under Ar for 0.5 h. CH₃I (8.48 mL, 136.17 mmol) was added dropwise to the mixture at 0° C. under Ar. The mixture was stirred at 25° C. under Ar for 16 h. After quenched with sat.NH₄Cl (50 mL), the mixture was extracted with ethyl acetate (100 mL*3). The combined organic phases were washed with brine (100 mL), dried over Na₂SO₄, filtered and concentrated in vacuo to give 26 (6 g, 53.22% yield) as yellow oil, which was used in the next step without further purification. MS-ESI (M+H⁺): 125.4. ¹H NMR (400 MHz, CDCl₃) δ 6.92 (d, J=0.7 Hz, 1H), 6.76 (d, J=0.9 Hz, 1H), 3.59 (s, 3H), 3.03-2.98 (m, 1H), 1.32 (d, J=6.9 Hz, 6H).

### 5-bromo-2-isopropyl-1-methyl-1H-imidazole (27)

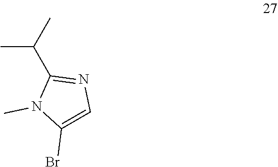

To the mixture of 26 (1.5 g, 12.08 mmol) in dichloromethane (15 mL) was added NBS (1.61 mL, 9.06 mmol) in MeCN (20 mL) dropwise at 0° C. under Ar. The mixture was stirred at 0° C. under Ar for 1 h. The mixture was quenched with aq. Na₂S₂O₅ (10 mL, 10%), extracted with ethyl acetate (30 mL*3). The organic phase was concentrated in vacuo and the residue was purified by flash column chromatograph (petroleum ether/ethyl acetate=10/1-5/1-2/1) to give 27 (1.64 g, 66.86% yield) as yellow solid. MS-ESI (M+H⁺+2): 204.9. ¹H NMR (400 MHz, CDCl₃) δ 6.91 (s, 1H), 3.53 (s, 3H), 2.99 (dt, J=13.7, 6.8 Hz, 1H), 1.30 (d, J=6.9 Hz, 6H).

### 2,5-dichloro-4-(2-isopropyl-1-methyl-1H-imidazol-5-yl)pyrimidine (28)

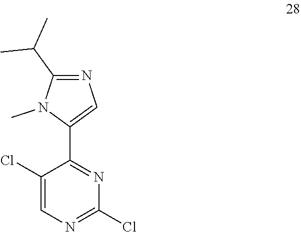 [SMILES: CC(C)c1ncc(-c2nc(Cl)ncc2Cl)n1C]

To the mixture of 27 (400 mg, 1.97 mmol) in THF (5 mL) was added isopropyl magnesium chloride (3.03 mL, 3.94 mmol, 1.3M in THF) at 0° C. under Ar. The mixture was stirred at 0° C. under Ar for 1 h. The mixture was transferred to the stirred solution of 2,4,5-trichloropyrimidine 23 (0.45 mL, 3.94 mmol) in THF (5 mL) at 0° C. under Ar. The resulting mixture was stirred at 25° C. under Ar for 16 h. The mixture was quenched with sat.NH₄Cl (10 mL), extracted with ethyl acetate (20 mL×3). The organic phase was dried over Na₂SO₄, filtered and concentrated in vacuo. The residue was purified by flash column chromatography (petroleum ether/ethyl acetate=5/1-2/1) to give 28 (125 mg, 23.41% yield) as yellow solid. MS-ESI (M+H)⁺: 271.2. ¹H NMR (400 MHz, CDCl₃) δ 7.68 (s, 1H), 7.43 (s, 1H), 4.00 (s, 3H), 3.11 (dt, J=13.6, 6.8 Hz, 1H), 1.37 (d, J=6.8 Hz, 6H).

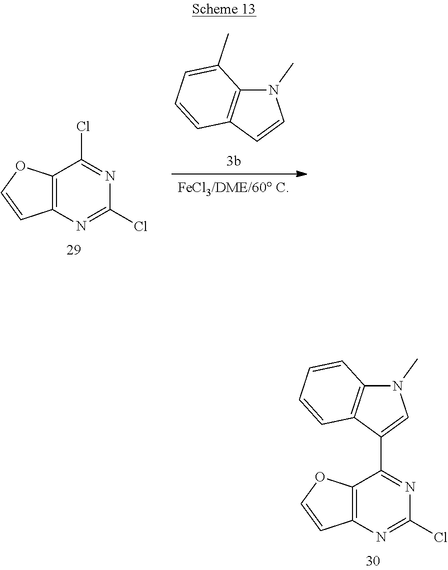 [SMILES: Clc1nc(Cl)c2occc2n1 ; Cc1cccc2ccn(C)c12 ; Cn1cc(-c2nc(Cl)nc3ccoc23)c2ccccc21]

### Intermediate 30: 2-chloro-4-(1-methyl-1H-indol-3-yl)furo[3,2-d]pyrimidine (30)

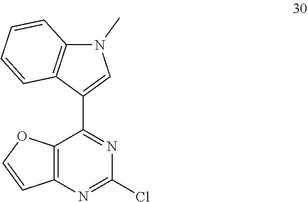 [SMILES: Cn1cc(-c2nc(Cl)nc3ccoc23)c2ccccc21]

To a solution of 29 (2.0 g, 10.58 mmol) in 25 mL of 1,2-dimethoxyethane was added 1-methyl-1H-indole 3b (2.8 g, 21.16 mmol), FeCl₃ (3.5 g, 21.16 mmol). The mixture was stirred at 60° C. for 16 h under Ar. After cooling to room temperature, the mixture was diluted with ethanol (25 mL) and H₂O (75 mL) and stirred for 2 h. The solid was collected by filtration and the filter cake was washed with water (100 mL). The solid was dried under vacuum to afford 30 (3.5 g, yield 81%). MS-ESI (M+H)⁺: 284.1. ¹H NMR (400 MHz, CDCl₃) δ 8.82-8.80 (m, 1H), 8.34 (s, 1H), 8.02 (d, J=8.0 Hz, 1H), 7.42-7.40 (m, 3H), 7.01 (s, 1H), 3.95 (s, 3H).

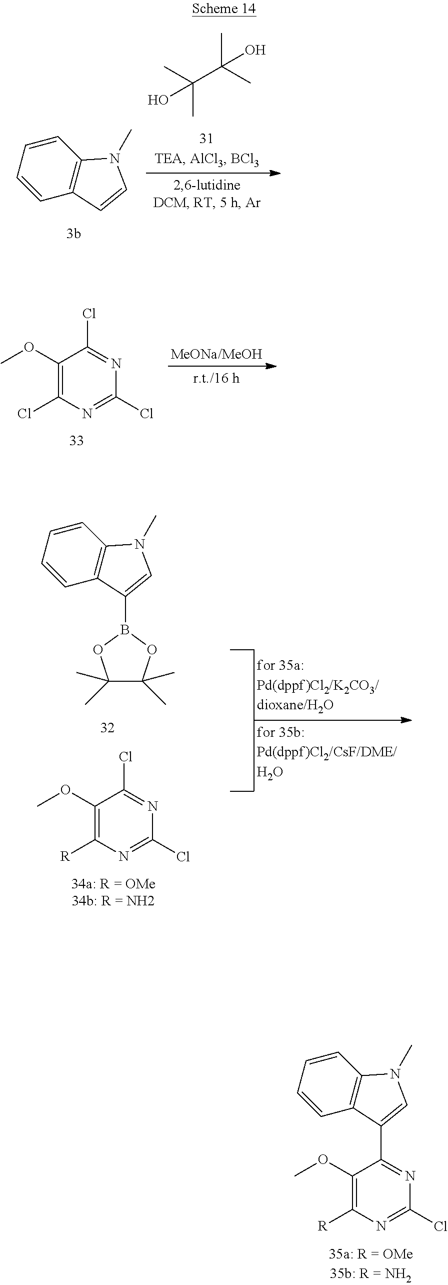

### Intermediate 35a: 1-methyl-3-(4, 4, 5, 5-tetramethyl-1, 3, 2-dioxaborolan-2-yl)-1H-indole (32)

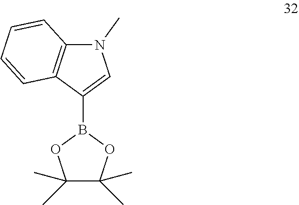 [SMILES: Cn1cc(B2OC(C)(C)C(C)(C)O2)c2ccccc21]

An oven dried 500 ml 3-necked round-bottomed flask cooled under N₂, was charged with AlCl₃ (6.1 g, 45.74 mmol). This was then suspended in CH₂Cl₂ (130 mL) and 2, 6-lutidine (4.9 g, 45.74 mmol) was added. To the resulting suspension was then added a solution of BCl₃ (1.0 M in dichloromethane, 46 ml, 45.74 mmol). After 30 minutes a homogeneous yellow solution was obtained to which was added 1-methyl-1H-indole 3b (5.0 g, 38.12 mmol). The reaction was stirred for 2.5 hours before being transferred by cannula under a positive pressure of N₂ to a solution of pinacol 31 (9.9 g, 83.86 mmol) in triethylamine (85 ml, 610 mmol) contained in an oven dried 100 mL round-bottomed flask, cooled under N₂, with care taken not to allow any significant rise in reaction temperature. After washing the flask with CH₂Cl₂ (20 mL×2), the volatiles were removed in vacuo. The resulting beige solid was suspended in petroleum ether (800 mL) and the solids was removed by filtration, upon washing with further pentane (100 mL×5). The extracts were combined and the volatiles removed in vacuo to give crude product, the residue was purified by flash column chromatography (petroleum ether/ethyl acetate=20/1) to afford 32 (8.2 g, yield 83.7%) as pale yellow solid. ¹H NMR (400 MHz, CD₃OD) δ 7.91 (d, J=8.0 Hz, 1H), 7.55 (s, 1H), 7.40 (d, J=8.0 Hz, 1H), 7.23-7.19 (m, 1H), 7.13-7.09 (m, 1H), 3.85 (s, 3H), 1.39 (s, 12H).

### 2, 4-dichloro-5, 6-dimethoxypyrimidine (34a)

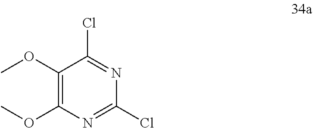

To a solution of 2,4,6-trichloro-5-methoxypyrimidine 33 (1.0 g, 4.68 mmol) in 20 mL of methanol was added MeONa (265 mg, 4.92 mmol) at 0° C. and the mixture was stirred at room temperature for 16 h. The mixture was concentrated under vacuum to give a residue. The residue was dissolved in 100 mL ethyl acetate and the mixture was stirred at room temperature for 30 min. After filtration, the filtrate was concentrated to afford 34a (915 mg, yield 93.45%). ¹H NMR (400 MHz, DMSO-d6) δ 4.02 (s, 3H), 3.83 (s, 3H).

### 3-(2-chloro-5, 6-dimethoxypyrimidin-4-yl)-1-methyl-1H-indole (35a)

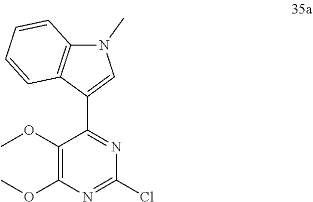 [SMILES: COc1nc(Cl)nc(-c2cn(C)c3ccccc23)c1OC]

To a solution of 34a (910 mg, 4.38 mmol), 32 (675 mg, 2.63 mmol) in 1,4-dioxane/water (20 mL, 4:1) were added PdCl₂ (dppf) (96 mg, 0.13 mmol) and K₂CO₃ (1.2 g, 8.75 mmol). The mixture was stirred at 110° C. under Ar for overnight. After cooling to room temperature, the mixture was diluted with ethyl acetate and the organic phase was washed with water. The organic phase was dried over Na₂SO₄, filtered and concentrated. The crude product was purified by flash column chromatography (petroleum ether/ethyl acetate=30/1) to give 35a as a green solid (260 mg, yield 19%). MS-ESI (M+H)⁺:304.1. ¹H NMR (400 MHz, CDCl₃) δ 8.57 (d, J=7.2 Hz, 1H), 7.99 (s, 1H), 7.36 (d, J=6.8 Hz, 1H), 7.33-7.28 (m, 2H), 4.21 (s, 3H), 3.90 (s, 3H), 3.86 (s, 3H).

### Intermediate 35b: 2, 6-dichloro-5-methoxypyrimidin-4-amine (34b)

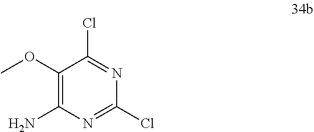

To a solution of 33 (3 g, 14 mmol) in DMSO (12 mL) was added ammonium hydroxide (25%, 1.6 mL, 21 mmol). The mixture was stirred at RT for 30 min. The mixture was poured into water (80 mL) to give a suspension. The suspension was filtered and the solid was washed with water to give a white solid. The solid was dried under vacuum to afford 34b (2.5 g, yield 91.5%). MS-ESI (M+H)⁺: 194.1.

### 2-chloro-5-methoxy-6-(1-methyl-1H-indol-3-yl)pyrimidin-4-amine (35b)

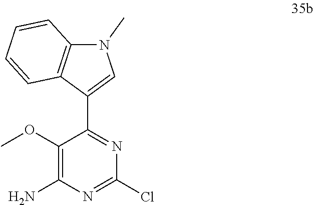 [SMILES: COc1c(N)nc(Cl)nc1-c1cn(C)c2ccccc12]

To a solution of 34b (500 mg, 2.58 mmol) in 1,2-dimethoxyethane (15 mL) and H₂O (5 mL) was added 32 (928 mg, 3.61 mmol), CsF (548 mg, 3.61 mmol) and Pd(dppf)₂Cl₂ (181 mg, 0.258 mmol). The mixture was stirred at 100° C. for 16 h under Ar. After concentration, the residue was purified by reverse phase column (0.5% NH₄OH/MeCN=0˜40%˜60%) to afford 35b (630 mg) as a green solid. MS-ESI (M+H)⁺:289.0.

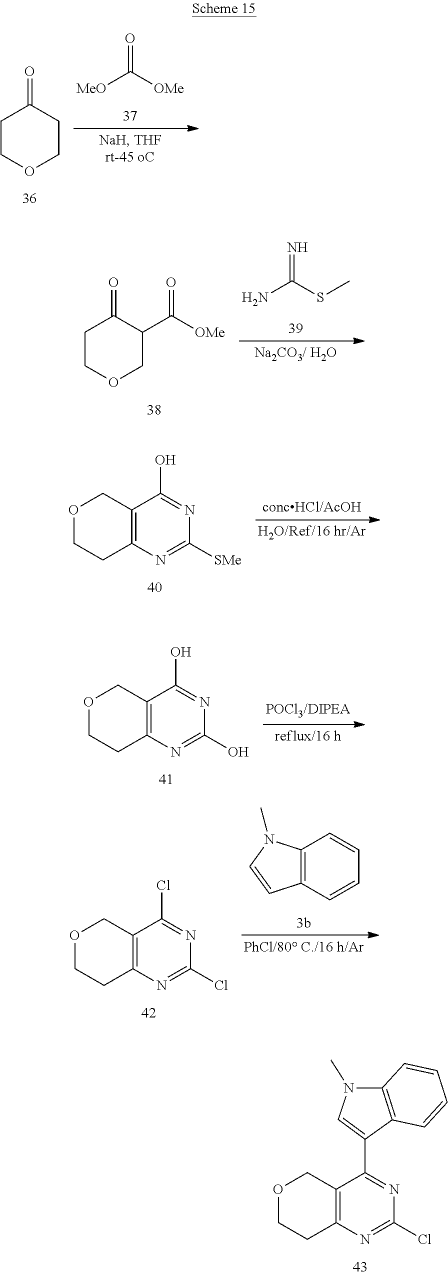

### Intermediate 43: methyl 4-oxotetrahydro-2H-pyran-3-carboxylate (38)

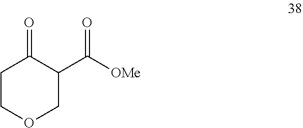 [SMILES: COC(=O)C1COCCC1=O]

To a solution of 36 (6.2 g, 62 mmol), dimethyl carbonate 37 (13.95 g, 155 mmol) in 150 mL of THF was added NaH (6.2 g, 155 mmol) at 0° C. and stirred at 0° C. for 30 min, then at room temperature for 30 min. Then the mixture was stirred at 40° C. under N₂ overnight. The mixture was cooled to 0° C. and added 250 mL 1.0 M HCl and 300 mL ethyl acetate. The two phases were separated. The organic layer was dried over Na₂SO₄, filtered and concentrated. The crude product was purified by flash column chromatography (petroleum ether/ethyl acetate=40/1-10/1) to afford 38 as a colorless oil (3.1 g, yield 31%).

### 2-(methylthio)-7, 8-dihy-dihyd-pyrano [4, 3-d]pyrimidin-4-ol (40)

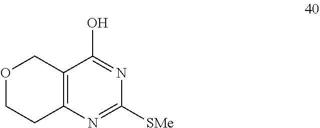 [SMILES: CSc1nc(O)c2c(n1)CCOC2]

To a solution of 38 (3.3 g, 11.77 mmol) in 100 mL H₂O was added Na₂CO₃ (2.5 g, 23.54 mmol), 39 (3.1 g, 19.6 mmol). The mixture was stirred at room temperature under darkness overnight. The mixture was filtered and the filter cake was washed with water (200 mL) and dried under vacuum to afford 40 as a white solid (1.7 g, yield 43.75%). MS-ESI (M+H)⁺: 199.1. ¹H NMR (400 MHz, DMSO-d6) δ 12.67 (s, 1H), 4.32 (s, 2H), 3.83 (t, J=5.6 Hz, 2H), 2.56 (t, J=5.6 Hz, 2H), 2.47 (s, 3H).

### 7, 8-dihydro-5H-pyrano [4, 3-d]pyrimidine-2, 4-diol (41)

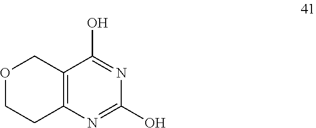 [SMILES: Oc1nc(O)c2c(n1)CCOC2]

To a solution of 40 (900 mg, 4.545 mmol) in 10 mL H₂O was added conc. HCl (1.0 mL, 4.545 mmol), AcOH (1.6 mL, 27.27 mmol). The mixture was stirred at 100° C. under Ar overnight. After cooling to room temperature, the mixture was filtered and the solid was dried under vacuum to afford 41 (600 mg, yield 78.5%). MS-ESI (M+H)⁺: 169.1. ¹H NMR (400 MHz, DMSO-d6) δ 11.00 (s, 1H), 10.86 (s, 1H), 4.19 (s, 2H), 3.77 (t, J=5.6 Hz, 2H), 2.39 (t, J=5.6 Hz, 2H).

### 2, 4-dichloro-7, 8-dihydro-5H-pyrano [4,3-d]pyrimidine (42)

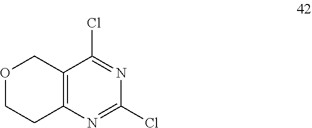 [SMILES: Clc1nc(Cl)c2c(n1)CCOC2]

To a solution of 41 (600 mg, 3.57 mmol) in 5 mL POCl₃ was added DIPEA (1.2 mL, 7.14 mmol). The mixture was stirred at 80° C. under Ar overnight. The mixture was concentrated to give a residue. With the addition of ice, the residue was neutralized with sat. NaHCO₃ to pH=7 and the resulting was extracted with ethyl acetate (100 mL*3). The combined organic layers were dried over Na₂SO₄, filtered and concentrated to give a crude product. The crude was purified by flash column chromatography (petroleum ether/ethyl acetate=10/1) to afford 42 (620 mg, yield 84.7%). MS-ESI (M+H)⁺: 240.0. ¹H NMR (400 MHz, DMSO-d6) δ 4.66 (t, J=8.8 Hz, 2H), 3.98-3.97 (m, 2H), 2.92-2.91 (m, 2H).

### 2-chloro-4-(1-methyl-1H-indol-3-yl)-7, 8-dihydro-5H-pyrano [4, 3-d]pyrimidine (43)

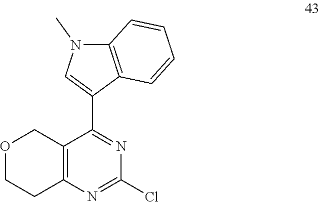 [SMILES: Cn1cc(-c2nc(Cl)nc3c2COCC3)c2ccccc21]

To a solution of 42 (520 mg, 2.536 mmol) in 15 mL of PhCl was added AlCl₃ (677 mg, 5.073 mmol). The mixture was stirred at room temperature for 2 h before addition of 1-methyl-1H-indole 3b (500 mg, 3.8 mmol). The resulting mixture was stirred at 80° C. for 16 h under Ar. After cooling to room temperature, the mixture was diluted with ethanol (15 mL) and H₂O (45 mL) and stirred at room temperature for 2 h. The solid was collected by filtration and the filter cake was washed with water (100 mL) and dried under vacuum to afford 43 (565 mg, yield 74.3%). MS-ESI (M+H)⁺: 300.1. ¹H NMR (400 MHz, CDCl₃) δ 8.60-8.56 (m, 1H), 7.40-7.33 (m, 3H), 7.28 (s, 1H), 4.85 (s, 2H), 4.07 (t, J=6.0 Hz, 2H), 3.89 (s, 3H), 3.00 (t, J=6.0 Hz, 2H).

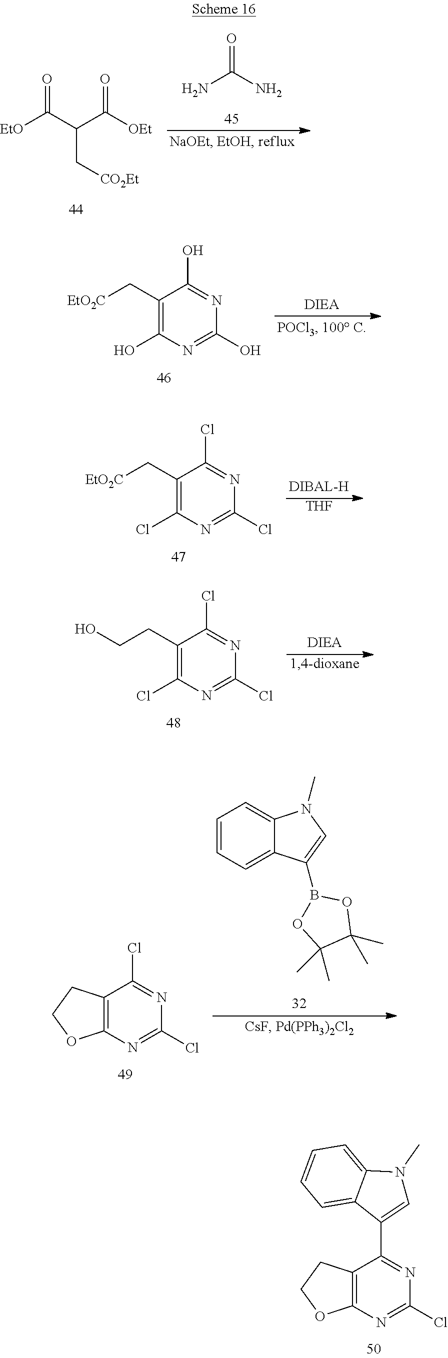

### Intermediate 50: ethyl 2-(2,4,6-trihydroxypyrimidin-5-yl)acetate (46)

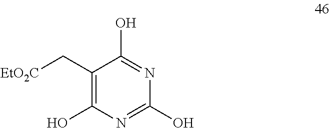 [SMILES: CCOC(=O)Cc1c(O)nc(O)nc1O]

To a solution of triethyl ethane-1,1,2-tricarboxylate 44 (100 g, 406.08 mmol) in anhydrous ethanol (800 mL) was added sodium ethoxide (27.63 g, 406.08 mmol) and urea 45 (24.39 g, 406.08 mmol). The mixture was stirred at reflux overnight. After cooling to room temperature, the mixture was evaporated to dryness. The residue was diluted with water and acidified with diluted hydrochloric acid (2N). The mixture was evaporated to dryness to give the desired product 46 (100 g, crude) as a pale-yellow solid. MS-ESI (M+H)⁺: 215.0. ¹H NMR (400 MHz, DMSO-d6) δ 9.17 (s, 1H), 9.01 (s, 2H), 3.95 (q, J=7.2 Hz, 2H), 3.00 (s, 2H), 1.25-1.15 (m, 3H)

### ethyl 2-(2,4,6-trichloropyrimidin-5-yl)acetate (47)

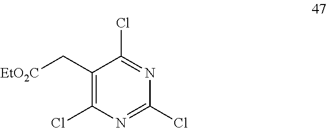 [SMILES: CCOC(=O)Cc1c(Cl)nc(Cl)nc1Cl]

To the mixture of 46 (100 g, 33.3 mmol) in POCl₃ (800 mL) were added DIEA (160 mL) dropwise. The mixture was stirred at 100° C. for 2 h. Most of POCl₃ was removed under reduced pressure and the residue was basified by aqueous sodium bicarbonate. The aqueous phase was extracted with ethyl acetate, and the organic extracts were combined, washed with brine, dried over anhydrous sodium sulfate and evaporated. The residue was purified by flash column chromatography (ethyl acetate/petroleum ether=0˜20%) to afford the desired product 47 (22 g, yield 17.48%) as a pale-yellow solid. ¹H NMR (400 MHz, CDCl₃) δ 4.30-4.16 (m, 2H), 3.94 (s, 2H), 1.33-1.24 (m, 3H). MS-ESI (M+H)⁺ 269.0.

### 2-(2,4,6-trichloropyrimidin-5-yl)ethan-1-ol (48)

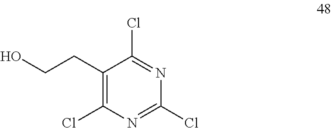 [SMILES: OCCc1c(Cl)nc(Cl)nc1Cl]

To the mixture of 47 (5.0 g, 18.55 mmol) in THF (50 mL) were added DIBAL-H (37.1 mL, 1 M). The mixture was stirred at 0° C. for 12 h. The mixture was quenched with saturated NH₄Cl solution. The aqueous phase was extracted with ethyl acetate, and the organic extracts were combined, washed by brine, dried over anhydrous sodium sulfate, evaporated and purified by flash column chromatography (ethyl acetate/petroleum ether=0-50%, in 50 min) to afford the desired product 48 (2.75 g, yield 65.16%) as a white solid. ¹H NMR (400 MHz, CDCl₃) δ 4.69 (s, 1H), 3.94 (t, J=6.8 Hz, 2H), 3.19 (t, J=6.8 Hz, 2H).

### 2,4-dichloro-5,6-dihydrofuro[2,3-d]pyrimidine (49)

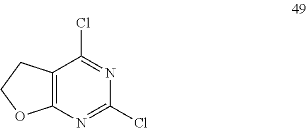 [SMILES: Clc1nc(Cl)c2c(n1)OCC2]

To a solution of 48 (5.0 g, 21.98 mmol) in 1,4-dioxane (750 mL) was added DIEA (11.49 mL). The mixture was stirred at 100° C. for 12 h. The mixture was concentrated and purified by flash column chromatography (ethyl acetate/petroleum ether=0-40%, in 30 min) to afford the desired product 49 (2.0 g, yield 47.63%) as a white solid. ¹H NMR (400 MHz, CDCl₃) δ 4.83 (t, J=8.8 Hz, 2H), 3.32 (t, J=8.8 Hz, 2H); MS-ESI (M+H)⁺: 191.1.

### 2-chloro-4-(1-methyl-1H-indol-3-yl)-5,6-dihydrofuro[2,3-d]pyrimidine (50)

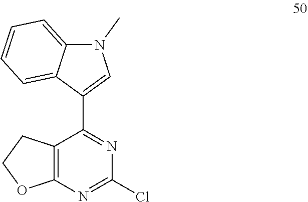 [SMILES: Cn1cc(-c2nc(Cl)nc3c2CCO3)c2ccccc21]

To a solution of 49 (2.0 g, 10.47 mmol) and 32 (2.69 g, 10.47 mmol) in DME (20 mL) and H₂O (2 mL) was added CsF (4.77 g, 31.41 mmol) and Pd(PPh₃)₂Cl₂ (670 0.07 mg, 1.05 mmol). The mixture was stirred at 100° C. for 12 h in microwave tube under N₂ atmosphere. After cooling to room temperature, the mixture was filtered and the filtrate was concentrated. The residue was purified by flash column chromatography (dichloromethane/methanol=100/1-20/1) to afford the desired product 50 (0.7 g, yield 23.40%) as a yellow solid. MS-ESI (M+H)⁺:286.1

### I. Syntheses of Aryl Amines/Intermediates

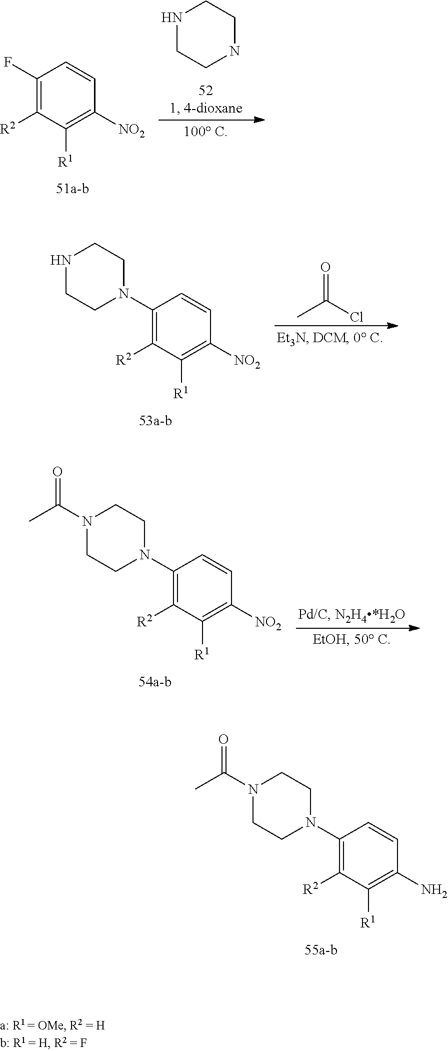

### Intermediate 55a: 1-(3-methoxy-4-nitrophenyl)piperazine (53a)

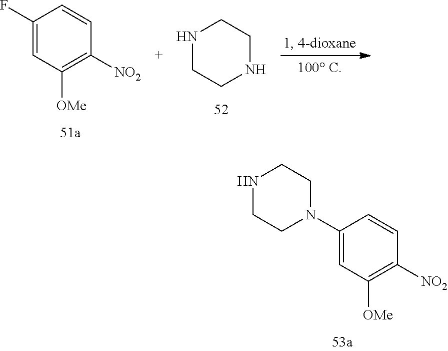 [SMILES: COc1cc(F)ccc1[N+](=O)[O-] ; C1CNCCN1 ; COc1cc(N2CCNCC2)ccc1[N+](=O)[O-]]

To a solution of piperazine 52 (8.1 g, 94.04 mmol) in 1,4-dioxane (150 mL), 4-fluoro-2-methoxy-1-nitrobenzene 51a (13.0 g, 75.97 mmol) was added. The mixture was stirred at 100° C. for 4 h. After cooling to room temperature, the mixture was concentrated under reduced pressure. The residue was diluted with dichloromethane (100 mL) and washed with H₂O (50 mL*2), dried with Na₂SO₄. After concentration, the residue was purified by flash column chromatography (dichloromethane/methanol=15/1) to give the desired product 53a (10.5 g, yield 47.1%) as a yellow solid.

### 1-(4-(3-methoxy-4-nitrophenyl)piperazin-1-yl)ethanone (54a)

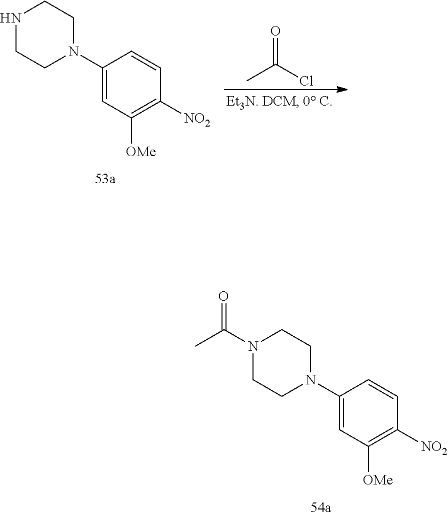

A solution of 53a (9.5 g, 40.04 mmol) and Et₃N (10.50 g, 103.76 mmol) in dichloromethane (50 mL) was stirred at 0° C. for 10 min. Then acetyl chloride (6.80 g, 86.63 mmol) was added dropwise and the mixture was stirred at 0° C. for another 1 h. The reaction mixture was quenched by saturated NaHCO₃ (5 mL) and diluted with dichloromethane (100 mL). The organic phase was washed with H₂O (50 mL*2), dried with Na₂SO₄. After concentration, the residue was purified by flash column chromatography (dichloromethane/methanol=12/1) to give the crude product 54a (12.6 g) as a yellow solid, which was used in the next step without further purification.

### 1-(4-(4-amino-3-methoxyphenyl)piperazin-1-yl)ethanone (55a)

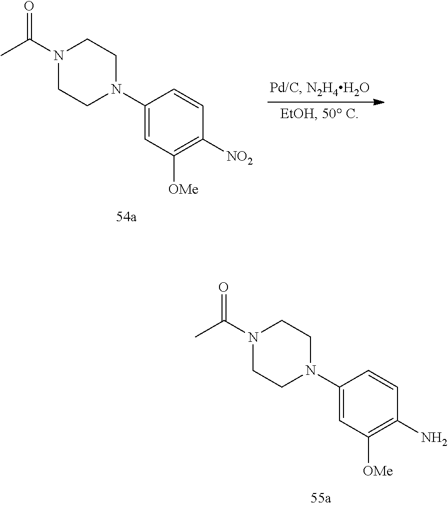 [SMILES: COc1cc(N2CCN(C(C)=O)CC2)ccc1[N+](=O)[O-] ; COc1cc(N2CCN(C(C)=O)CC2)ccc1N]

To a solution of 54a (12.6 g, 45.11 mmol) in ethanol (100 mL), 5% Pd/C (1.84 g, w %=15%) and hydrazine hydrate (80%, 50 mL) were added. The mixture was stirred at 50° C. for 3 h. Then the mixture was concentrated under reduced pressure. The residue was diluted with ethyl acetate (150 mL). The organic phase was washed with H₂O (50 mL*2), dried with Na₂SO₄. After concentration, the residue was purified by flash column chromatography (dichloromethane/methanol=15/1) to give the product 55a (8.3 g, total yield 83.2% over two steps) as an off-white solid.

### Intermediate 55b: 1-(2-fluoro-4-nitrophenyl)piperazine (53b)

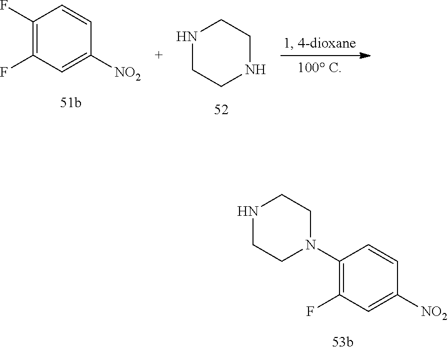 [SMILES: O=[N+]([O-])c1ccc(F)c(F)c1 ; C1CNCCN1 ; O=[N+]([O-])c1ccc(N2CCNCC2)c(F)c1]

Synthesis of 53b was similar to that of 53a. Using piperazine (10.21 g, 118.53 mmol) and 1,2-difluoro-4-nitrobenzene (13.47 g, 84.67 mmol) to give 53b as a yellow oil (14.0 g, yield 52.4%).

### 1-(4-(2-fluoro-4-nitrophenyl)piperazin-1-yl)ethanone (54b)

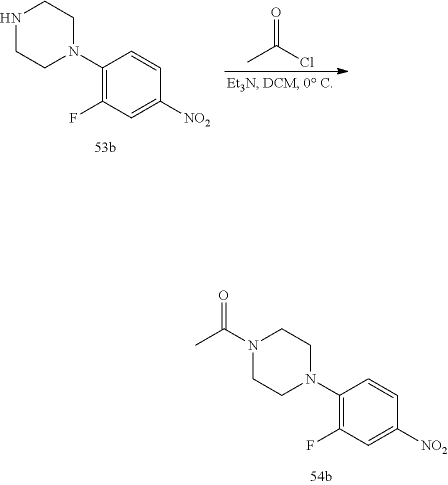

Synthesis of 54b was similar to that of 54a. Using 53b (14.0 g, 62.16 mmol) and acetyl chloride (7.81 g, 99.49 mmol) to give 54b as a yellow solid (15.6 g, yield 93.9%).

### 1-(4-(4-amino-2-fluorophenyl)piperazin-1-yl)ethanone (55b)

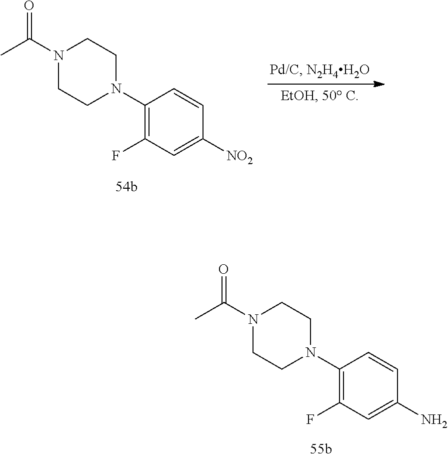 [SMILES: CC(=O)N1CCN(c2ccc([N+](=O)[O-])cc2F)CC1 ; CC(=O)N1CCN(c2ccc(N)cc2F)CC1]

Synthesis of 55b was similar to that of 55a. Using 54b (15.12 g, 56.57 mmol) to give 55b as a light yellow solid (13.1 g, yield 97.6%).

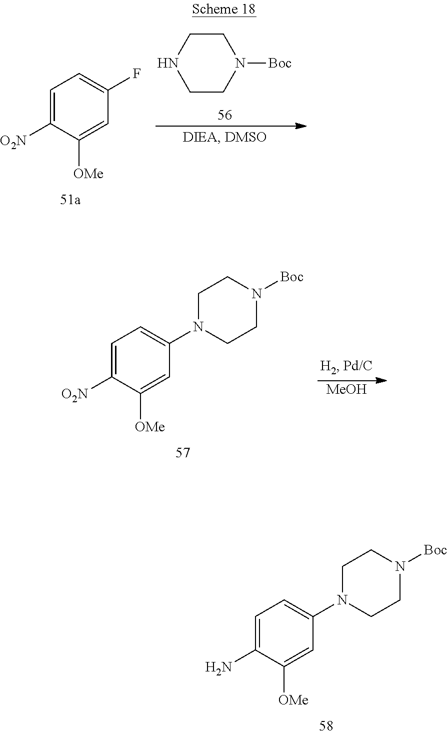

### Intermediate 58: tert-butyl 4-(3-methoxy-4-nitrophenyl)piperazine-1-carboxylate (57)

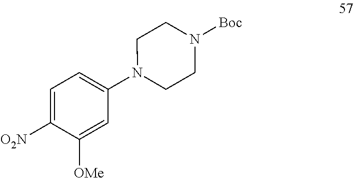 [SMILES: COc1cc(N2CCN(C(=O)OC(C)(C)C)CC2)ccc1[N+](=O)[O-]]

A solution of tert-butyl piperazine-1-carboxylate (3.37 g, 18.12 mmol), 51a (3.10 g, 18.12 mmol) and N,N-diisopropyl ethylamine (6.31 mL, 36.2 mmol) in DMSO (11 mL) was at 95° C. for 20 h in a sealed tube. Upon cooling, the reaction mixture crystallized to a yellow solid. It was diluted with 150 mL of ethyl acetate, washed sequentially with 20 mL of water, 20 mL of NaHCO₃ and brine (20 mL). The organic solution was dried over MgSO₄ and concentrated to afford 57 (5.0 g, yield 82%) as a yellow crystalline solid. (MS-ESI) (M+H)⁺: 338.0. ¹H NMR (400 MHz, CDCl₃) δ 8.00 (dd, J=9.3, 3.2 Hz, 1H), 6.41 (d, J=9.3 Hz, 1H), 6.31 (s, 1H), 3.95 (d, J=1.6 Hz, 3H), 3.63-3.58 (m, 4H), 3.42-3.37 (m, 4H), 1.49 (s, 9H).

### tert-butyl 4-(4-amino-3-methoxyphenyl)piperazine-1-carboxylate (58)

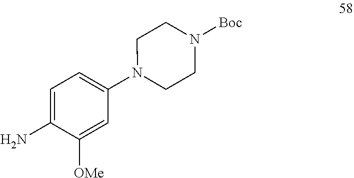 [SMILES: COc1cc(N2CCN(C(=O)OC(C)(C)C)CC2)ccc1N]

Pd/C (10%, 1.4 g, 1.34 mmol) and 57 (3.0 g, 8.9 mmol) were treated with methanol (30 mL) and allowed to stir under an atmosphere of hydrogen (balloon) for 23 h. The reaction mixture was filtered and the resulting purple solution was then concentrated under vacuum to afford 58 (2.9 g, 9.2 mmol) as a yellow solid, which was used to next step directly without further purification. (MS-ESI) (M+H)⁺: m/z 307.9

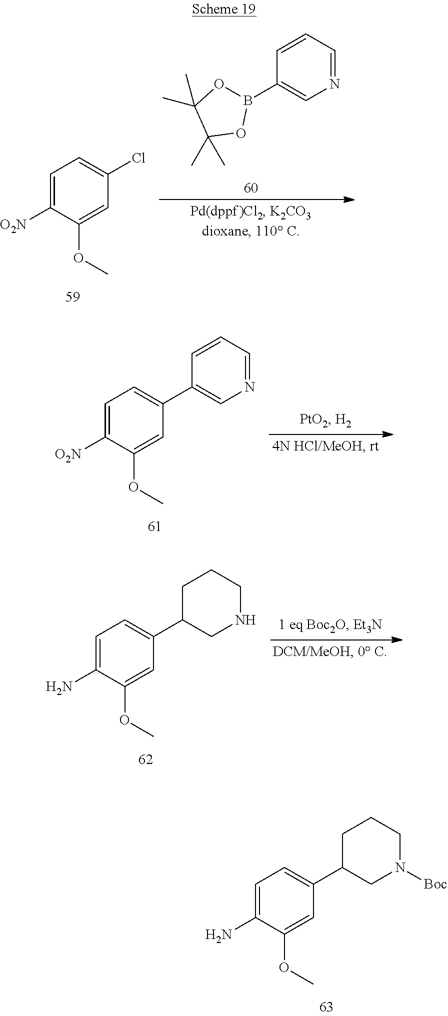 [SMILES: COc1cc(Cl)ccc1[N+](=O)[O-] ; CC1(C)OB(c2cccnc2)OC1(C)C ; COc1cc(-c2cccnc2)ccc1[N+](=O)[O-] ; COc1cc(C2CCCNC2)ccc1N ; COc1cc(C2CCCN(C(=O)OC(C)(C)C)C2)ccc1N]

### Intermediate 63: 3-(3-methoxy-4-nitrophenyl)pyridine (61)

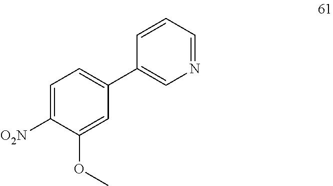 [SMILES: COc1cc(-c2cccnc2)ccc1[N+](=O)[O-]]

To the solution of 59 (5 g, 26.71 mmol) and 3-(4,4,5,5-tetramethyl-1,3,2-dioxaborolan-2-yl)pyridine 60 (10.9 g, 53.3 mmol) in 1,4-dioxane (125 mL) and H₂O (5 mL) were added Pd(dppf)Cl₂ (980 mg, 1.3 mmol) and K₂CO₃ (7.37 g, 53.3 mmol). The mixture was stirred at 110° C. for 16 h. The mixture was concentrated and the residue was purified by flash column chromatography (petroleum ether/ethyl acetate=5/1-2/1-1/1) to give 61 (3 g, 48.89% yield) as white solid. MS-ESI (M+H)⁺: 231.0. ¹H NMR (400 MHz, CDCl₃) δ 8.86 (s, 1H), 8.69 (s, 1H), 7.99 (d, J=8.2 Hz, 1H), 7.90 (d, J=7.9 Hz, 1H), 7.44 (dd, J=7.6, 4.9 Hz, 1H), 7.24-7.20 (m, 2H), 4.05 (s, 3H).

### 2-methoxy-4-(piperidin-3-yl)aniline (62)

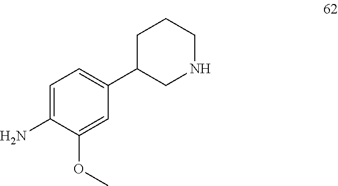

To the mixture of 61 (1 g, 4.34 mmol) in methanol (20 mL) were added PtO₂ (99 mg, 0.43 mmol) and conc. HCl (2 mL, 24 mmol). The mixture was stirred at 25° C. under H₂ for 16 h. The mixture was filtered and concentrated to give 62 (1 g, ˜100% yield) as yellow oil, which was used in the next step without further purification. MS-ESI (M+H)⁺: 207.1.

### tert-butyl 3-(4-amino-3-methoxyphenyl)piperidine-1-carboxylate (63)

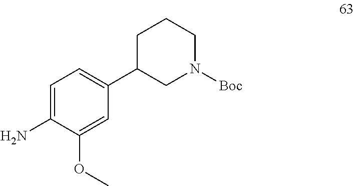 [SMILES: COc1cc(C2CCCN(C(=O)OC(C)(C)C)C2)ccc1N]

To the mixture of 62 (1 g, 8.73 mmol) in methanol (20 mL) and dichloromethane (20 mL) were added Et₃N (1.21 mL, 0.43 mmol) and Boc₂O (952 mg, 4.36 mmol) at 0° C. under Ar. The mixture was stirred at 0° C. under Ar for 2 h. The mixture was quenched with H₂O (1 mL) and concentrated. The residue was purified by flash column chromatography (petroleum ether/ethyl acetate=10/1-5/1) to give 63 (535 mg, 40.02% yield) as yellow oil. MS-ESI (M+H)⁺: 307.3.

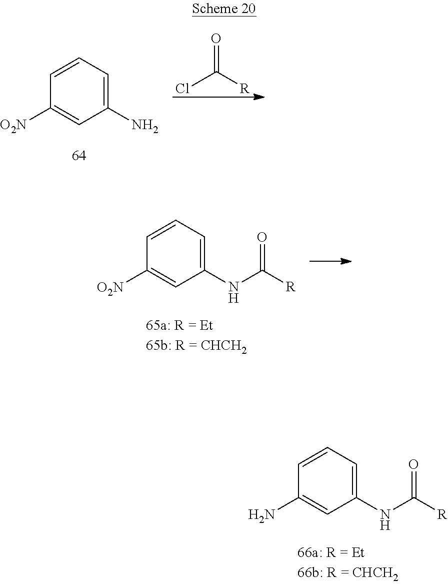

### Intermediate 66a: N-(3-nitrophenyl)propionamide (65a)

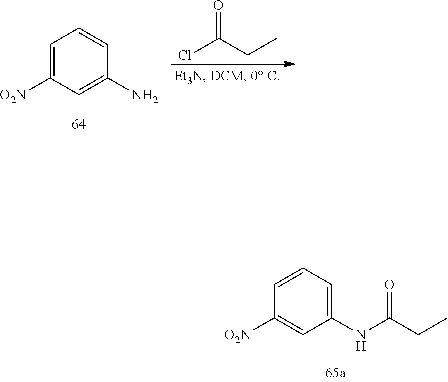

A mixture of 3-nitroaniline 64 (10.12 g, 73.27 mmol), Et₃N (10.11 g, 100.10 mmol) in dichloromethane (100 mL) was stirred at 0° C. for 30 min. Then propionyl chloride (7.78 g, 85.06 mmol) was added dropwise and the mixture was stirred at 0° C. for another 1 h. The reaction mixture was quenched by saturated NaHCO₃ (30 mL) and diluted with dichloromethane (100 mL). The organic phase was washed with H₂O (50 mL*2), dried with Na₂SO₄. The organic phase was concentrated under reduced pressure to give the crude product 65a (12.62 g, yield 88.7%) as a gray solid, which was used in the next step without further purification.

### N-(3-aminophenyl)propionamide (66a)

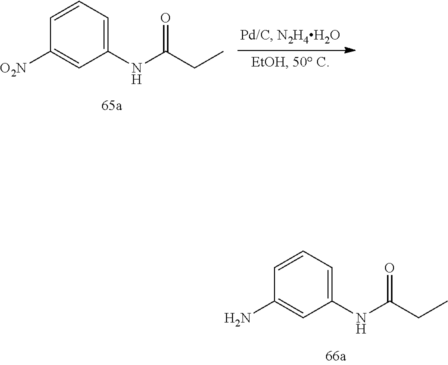

To a solution of 65a (12.0 g, 61.80 mmol) in ethanol (40 mL), 5% Pd/C (1.25 g, w %=10%) and hydrazine hydrate (80%, 50 mL) were added. The mixture was stirred at 60° C. for 6 h. The mixture was filtered off and the filtrate was concentrated under reduced pressure. The residue diluted with ethyl acetate (150 mL). The organic phase was washed with H₂O (50 mL*2), dried with Na₂SO₄. The organic phase was concentrated to give the desired product 66a (8.21 g, yield 83.2%) as a reddish-brown oil.

### Intermediate 66b: N-(3-nitrophenyl)acrylamide (65b)

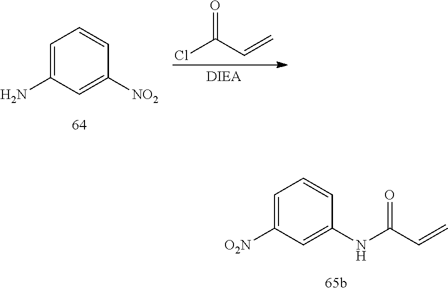

To a reactor (250 mL) was charged with 64 (27 g, 0.2 mol), N,N-diisopropyl ethylamine (50 g, 0.4 mol) and tetrahydrofuran (120 mL). Then the reactor was cooled with ice-bath. When the reactor internal temperature was reduced to −3° C., acryloyl chloride (27 g, 0.3 mol) dissolved in tetrahydrofuran (20 m L) was dropwise added over 1 h. The reaction mixture was stirred for 0.5 h. Aqueous NaOH (1M, 100 ml) was dropped to the reaction mixture. And then the reaction mixture was stirred for 0.5 h. The tetrahydrofuran layer was separated, and the aqueous layer was extracted with ethyl acetate (100 mL). The combined organic phases was concentrated. The crude 65b was dissolved in ethyl acetate (500 ml), and then washed with water (100 ml*3). The ethyl acetate solution was evaporated slowly under vacuum. The crude 65b with 100 ml ethyl acetate was heated to reflux for dissolution. And then the petroleum ether 150 ml was dropped to the solution. The solution was cooled to room temperature to precipitate the product 65b (32.9 g, 85.6% yield).

### N-(3-aminophenyl)acrylamide(66b)

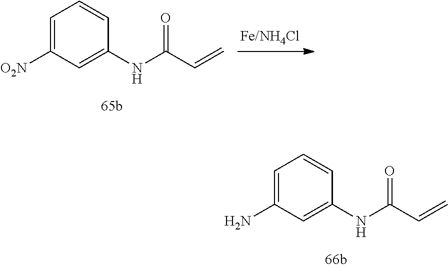

The compound 65b (28.8 g, 150 mmol) was dissolved in tetrahydrofuran (200 ml). Then water (80 ml), NH₄Cl (32.1 g, 600 mmol) and iron powder (42 g, 750 mmol) were added to the flask. The reaction solution was heated to reflux for 2 h. The reaction mixture was filtered, and the filtrate was concentrated. The crude 3 was dissolved in ethyl acetate (400 ml) and adjusted the pH with aqueous NaHCO₃, and then washed by water (100 ml*3). The ethyl acetate solution was evaporated slowly under vacuum to obtain the crude 66b, and then purified by flash column chromatography to obtained the product 66b (16.5 g, 67.6% yield).

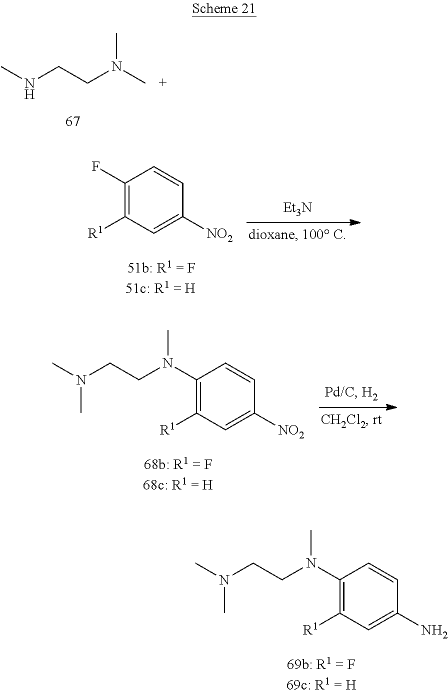

### Intermediate 69b: N¹-(2-fluoro-4-nitrophenyl)-N¹,N²,N²-trimethylethane-1,2-diamine (68b)

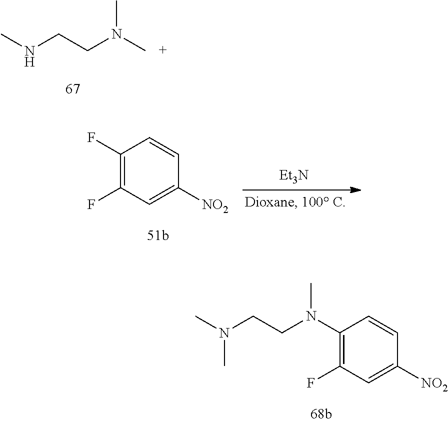

A mixture of 1,2-difluoro-4-nitrobenzene 51b (2.95 g, 18.54 mmol), N¹,N¹,N²-trimethylethane-1,2-diamine 67 (2.05 g, 20.06 mmol) and Et₃N (3.20 g, 31.69 mmol) in dioxane (10 mL) was stirred at 100° C. for 3 h. After cooling to room temperature, the mixture was concentrated under reduced pressure. The residue was diluted with dichloromethane (100 mL) and washed with H₂O (40 mL*2), dried with Na₂SO₄. After concentration, the residue was purified by flash column chromatography (dichloromethane/methanol=20/1) to give the product 68b (3.6 g, yield 74.4%) as a yellow oil.

### N¹-(2-(dimethylamino)ethyl)-2-fluoro-N¹-methylbenzene-1,4-diamine (69b)

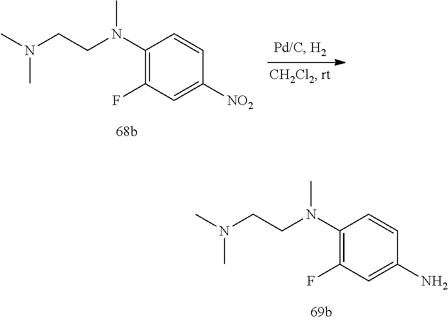 [SMILES: CN(C)CCN(C)c1ccc([N+](=O)[O-])cc1F ; "Pd/C, H2" ; CN(C)CCN(C)c1ccc(N)cc1F]

A mixture of 68b (3.5 g, 14.51 mmol) and 5% Pd/C (0.68 g, W %=19%) in dichloromethane (10 mL) was stirred at 0° C. under H₂ overnight. The reaction mixture was filtered off and the filtrate was concentrated under reduced pressure to give the crude product 69b (2.0 g, yield 65.4%) as a brown oil, which was used in the next step without further purification.

### Intermediate 69c: N¹,N¹,N²-trimethyl-N²-(4-nitrophenyl)ethane-1,2-diamine (68c)

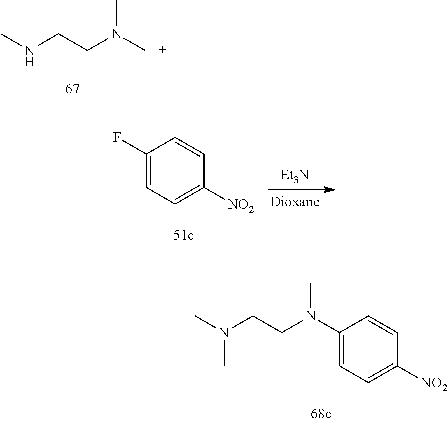

A mixture of 1-fluoro-4-nitrobenzene 51c (3.96 g, 28.07 mmol), N¹,N¹,N²-trimethylethane-1,2-diamine 67 (3.05 g, 29.85 mmol) and Et₃N (5.5 g, 54.4 mmol) in dioxane (20 mL) was stirred at 90° C. for 7.5 h. After cooling to room temperature, the mixture was concentrated under reduced pressure. The residue was diluted with ethyl acetate (100 mL) and washed with H₂O (40 mL*2), dried with Na₂SO₄. After concentration, the residue was purified by flash column chromatography (dichloromethane/methanol=20/1) to give the product 68c (5.50 g, yield 82.5%) as a yellow oil.

### N¹-(2-(dimethylamino)ethyl)-N¹-methylbenzene-1,4-diamine (69c)

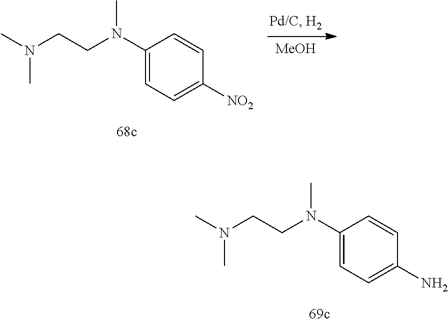 [SMILES: "MeOH" ; CN(C)CCN(C)c1ccc([N+](=O)[O-])cc1 ; "Pd/C, H2" ; CN(C)CCN(C)c1ccc(N)cc1]

A mixture of 68c (5.50 g, 24.63 mmol) and 5% Pd/C (0.54 g, w %=9.8%) in methanol (50 mL) was stirred at room temperature for 4.5 h. The reaction mixture was filtered off and the filtrate was concentrated under reduced pressure to give the crude product 69c (4.30 g, yield 90.3%) as a brown oil, which was used in the next step without further purification.

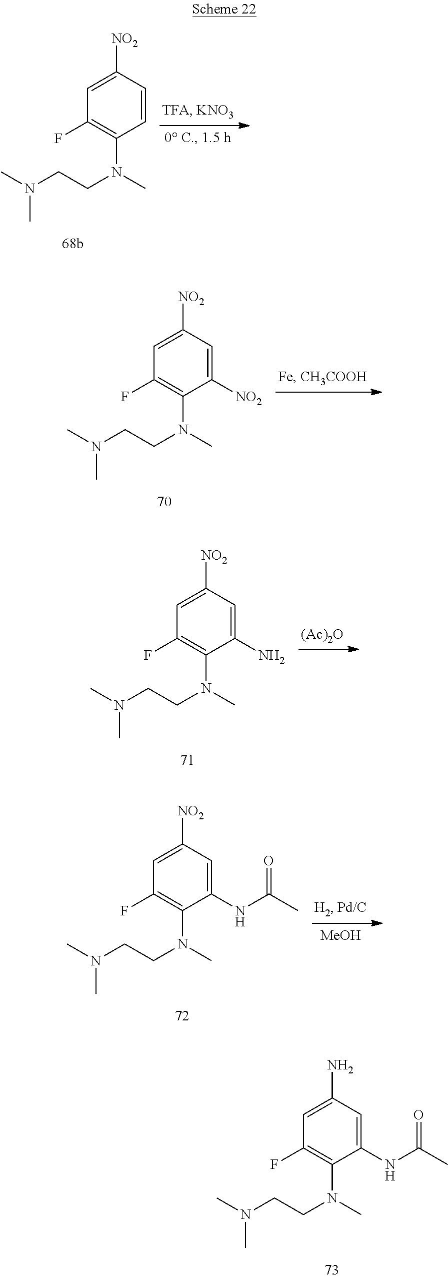

### Intermediate 73: N¹-(2-fluoro-4,6-dinitrophenyl)-N¹,N²,N²-trimethylethane-1,2-diamine (70)

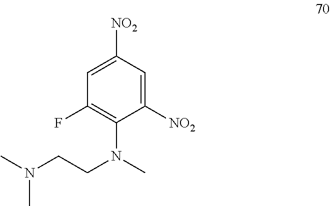 [SMILES: CN(C)CCN(C)c1c(F)cc([N+](=O)[O-])cc1[N+](=O)[O-]]

A solution of 68b (5.0 g, 20.75 mmol) in TFA (50 mL) was stirred at 0° C. for 10 min. Then KNO₃ (4.2 g, 41.49 mmol) was added and the mixture was stirred at 0° C. for another 1.5 h. The reaction mixture was diluted with dichloromethane and evaporated under vacuum. Then the residue was diluted with dichloromethane and filtered. The organic phase was concentrated to give crude 70, which was used to next step directly without further purification.

### N¹-(2-(dimethylamino)ethyl)-6-fluoro-N¹-methyl-4-nitrobenzene-1,2-diamine (71)

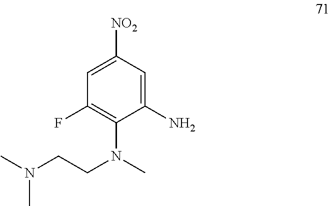 [SMILES: CN(C)CCN(C)c1c(N)cc([N+](=O)[O-])cc1F]

To a solution of 70 (1.19 g, 4.15 mmol) in acetic acid (15 mL), iron powder (930 mg, 8.6 mmol) was added. The mixture was stirred at room temperature overnight. After concentration, the residue was purified by flash column chromatography (dichloromethane to dichloromethane/methanol=50/1) to give 71 as a yellow solid (700 mg, yield 66%). MS-ESI (M+H)⁺: 257.1. ¹H NMR (400 MHz, DMSO-d6) δ 7.39 (s, 1H), 7.18 (d, J=7.2 Hz, 1H), 6.20 (s, 2H), 3.28 (br, 4H), 2.75 (s, 6H), 2.60 (s, 3H).

### N-(2-((2-(dimethylamino)ethyl)(methyl)amino)-3-fluoro-5-nitrophenyl)acetamide (72)

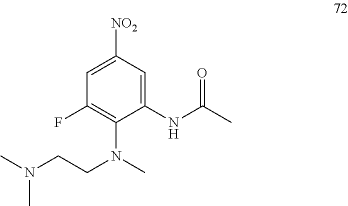 [SMILES: CC(=O)Nc1cc([N+](=O)[O-])cc(F)c1N(C)CCN(C)C]

A solution of 71 (0.7 g, 2.7 mmol) in Ac₂O (5 mL) was stirred at room temperature for overnight. The mixture was concentrated under vacuum and the residue was triturated with Et₂O (10 mL). The solid was collected by filtration and dried under vacuum to give 72 as a yellow solid (440 mg, 54%). MS-ESI (M+H)⁺: 299.1. ¹H NMR (400 MHz, DMSO-d6) δ 9.68 (s, 1H), 8.99 (s, 1H), 7.86 (d, J=7.2 Hz, 1H), 3.35-3.32 (m, 4H), 2.80 (s, 6H), 2.70 (s, 3H), 2.25 (s, 3H).

### N-(5-amino-2-((2-(dimethylamino)ethyl)(methyl)amino)-3-fluorophenyl)acetamide (73)

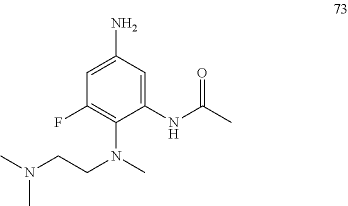 [SMILES: CC(=O)Nc1cc(N)cc(F)c1N(C)CCN(C)C]

To a solution of 72 (1.0 g, 3.3 mmol) in tetrahydrofuran/ethyl acetate=1/1 (20 mL) was added Pd/C (120 mg). The mixture was stirred at room temperature for 1.5 h under hydrogen atmosphere (balloon pressure). The catalyst was filtered off and the filtrate was concentrated under vacuum to give 73 as a light brown solid (840 mg, yield 93%). MS-ESI (M+H)⁺: 269.1. ¹H NMR (400 MHz, DMSO-d6) δ 9.08 (s, 1H), 7.41 (s 1H), 6.04 (d, J=9.6 Hz, 1H), 5.36 (s, 2H), 3.14 (br, 4H), 2.78 (s, 6H), 2.55 (s, 3H), 2.14 (s, 3H).

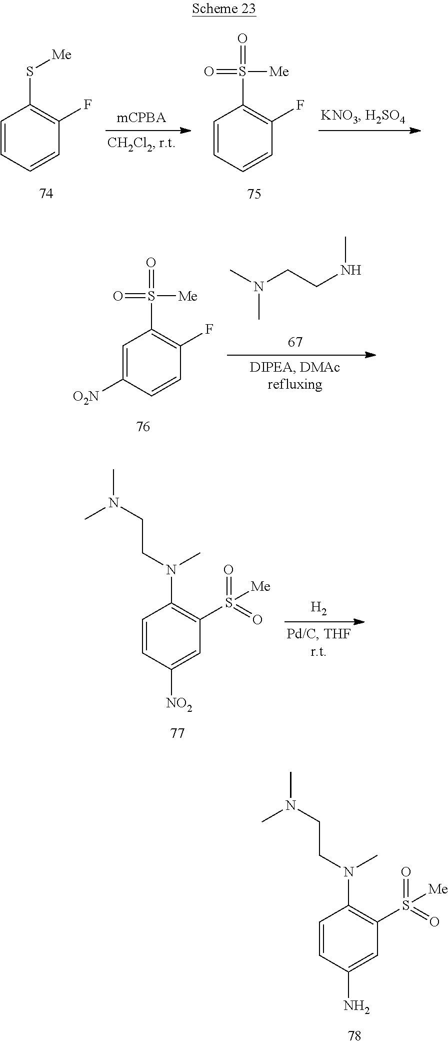

### Intermediate 78: 1-fluoro-2-(methylsulfonyl)benzene (75)

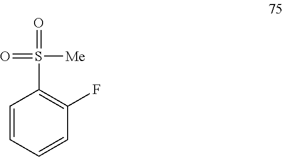 [SMILES: CS(=O)(=O)c1ccccc1F]

3-Chloroperoxybenzoic acid (10.3 g, 59.7 mmol) was added in portions to a solution of 2-Fluorothioanisole 74 (2.8 g, 19.7 mmol) in dichloromethane (20 ml) at 0° C. The mixture was allowed to warm to room temperature and stirred overnight. The reaction was quenched with Na₂SO₃ aqueous. and the mixture was extracted with dichloromethane. The organic layer was washed with saturated NaHCO₃ aqueous, and concentrated in vacuo to give the title compound 75 (4.0 g), which was used directly in the next step without further purification.

### 1-fluoro-2-(methylsulfonyl)-4-nitrobenzene (76)

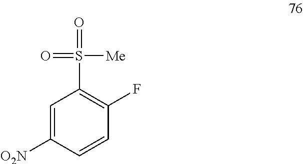 [SMILES: CS(=O)(=O)c1cc([N+](=O)[O-])ccc1F]

A mixture of 1-fluoro-2-(methylsulfonyl)benzene 75 (2.0 g, 11.5 mmol) and conc. H₂SO₄ (15 ml) was cooled with ice-salt bath and stirred for 10 mins. KNO₃ (3.2 g, 31.7 mmol) was added in portions to the mixture while maintaining the temperature below 20° C. The mixture was stirred for 0.5 h and then poured into ice-water. The mixture was extracted with ethyl acetate. The organic layer was separated, washed with water, concentrated in vacuo to afford a yellow solid. This solid was then stirred in ethyl acetate (5 ml) and petroleum ether (5 ml) for 1 h followed by filtration. The filter cake was collected and dried to give the title compound 76 (1.8 g, yield 83.4% over two steps) as a yellow solid.

### N¹,N¹,N²-trimethyl-N²-(2-(methylsulfonyl)-4-nitrophenyl)ethane-1,2-diamine (77)

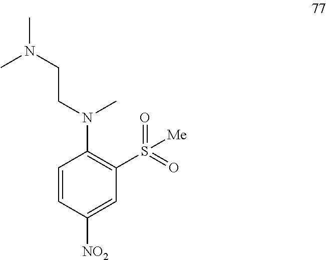 [SMILES: CN(C)CCN(C)c1ccc([N+](=O)[O-])cc1S(C)(=O)=O]

A mixture of 1-fluoro-2-(methylsulfonyl)-4-nitrobenzene 76 (1.0 g, 4.6 mmol), N,N,N′-trimethyl ethylenediamine 67 (0.93 g, 9.1 mmol), N,N-diisopropyl ethylamine (1.47 g, 11.4 mmol) in N,N-dimethylacetamide (10 ml) was heated at refluxing temperature for 2.5 h then cooled to room temperature. Water was added and the mixture was extracted with ethyl acetate. The organic layer was separated, washed with water, dried over Na₂SO₄, filtered and concentrated in vacuo to give the title compound 77 (1.50 g) as an oil, which was used directly in the next step without further purification.

### N¹-(2-(dimethylamino)ethyl)-N¹-methyl-2-(methylsulfonyl)benzene-1,4-diamine (78)

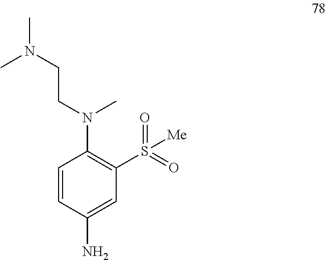 [SMILES: CN(C)CCN(C)c1ccc(N)cc1S(C)(=O)=O]

A mixture of N¹,N¹,N²-trimethyl-N²-(2-(methylsulfonyl)-4-nitrophenyl)ethane-1,2-diamine 77 (1.50 g, 5.0 mmol) and Pd/C (10% on activated carbon, 0.143 g, 0.13 mmol) in tetrahydrofuran (20 ml) was hydrogenated with hydrogen balloon at room temperature for 2 h. After completion of the reaction, the reaction mixture was filtered through Celite®. The filtrate was concentrated in vacuo to give the title compound 78 (1.5 g) as an oil, which was directly used in the next step without further purification.

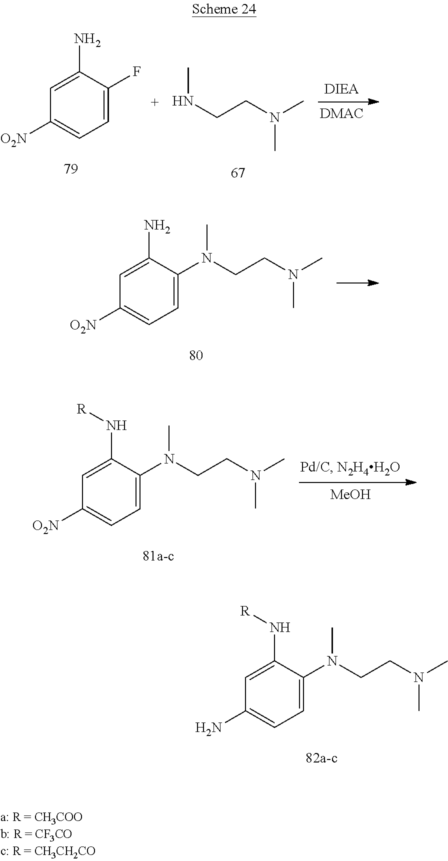

### Intermediate 82a: N¹-(2-(dimethylamino)ethyl)-N¹-methyl-4-nitrobenzene-1,2-diamine (80)

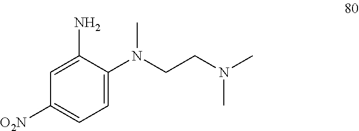 [SMILES: CN(C)CCN(C)c1ccc([N+](=O)[O-])cc1N]

A mixture of 2-fluoro-5-nitroaniline 79 (7.99 g, 51.18 mmol), N¹,N¹,N²-trimethylethane-1,2-diamine 67 (7.63 g, 74.67 mmol) and N,N-diisopropyl ethylamine (8.01 g, 61.98 mmol) in DMAC (20 mL) was stirred at 100° C. overnight. After cooling to room temperature, the mixture was diluted with H₂O (50 mL) and extracted with ethyl acetate (70 mL*3). The organic phase was merged and washed with H₂O (50 mL*3), dried with Na₂SO₄. After concentration, the residue was purified by flash column chromatography (dichloromethane/methanol=20/1) to give the product 80 (10.6 g, yield 86.9%) as a yellow oil.

### N-(2-((2-(dimethylamino)ethyl)(methyl)amino)-5-nitrophenyl) acetamide (81a)

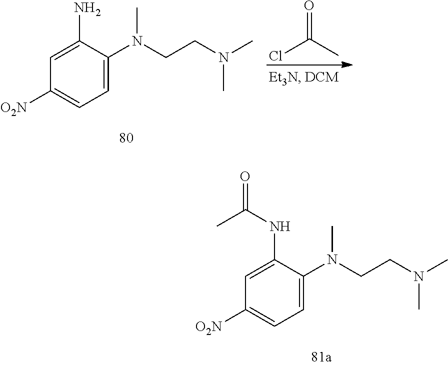

A solution of 80 (10.01 g, 42.01 mmol) and Et₃N (8.45 g, 83.51 mmol) in dichloromethane (50 mL) was stirred at 0° C. for 25 min. Then acetyl chloride (5.12 g, 65.22 mmol) was added dropwise and the mixture was stirred at 0° C. for another 1 h. The reaction mixture was quenched by saturated NaHCO₃ (30 mL) and diluted with ethyl acetate (100 mL). The organic phase was washed with H₂O (40 mL*3). After concentration, the residue was dried in an air-circulating oven at 45° C. to give the product 81a (8.68 g, yield 73.7%) as a light yellow solid.

### N-(5-amino-2-((2-(dimethylamino)ethyl)(methyl)amino) phenyl)acetamide (82a)

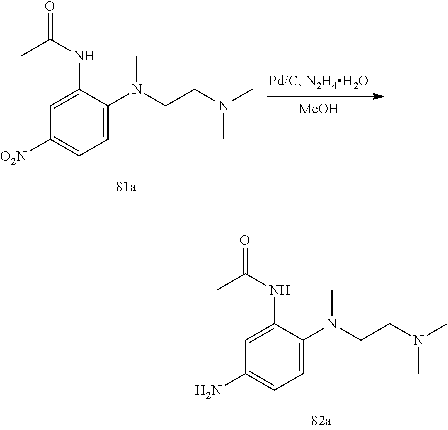 [SMILES: "MeOH" ; CC(=O)Nc1cc([N+](=O)[O-])ccc1N(C)CCN(C)C ; CC(=O)Nc1cc(N)ccc1N(C)CCN(C)C]

To a solution of 81a (2.12 g, 7.56 mmol) in methanol (20 mL), 5% Pd/C (0.25 g, w %=12%) and hydrazine hydrate (80%, 15 mL) were added. The mixture was stirred at 60° C. for 2.5 h. The mixture was filtered off and the filtrate was concentrated under reduced pressure. The residue diluted with ethyl acetate (100 mL). The organic phase was washed with H₂O (30 mL*3), dried with Na₂SO₄. The organic phase was concentrated to give the product 82a (1.80 g, yield 95.1%) as an off-white solid.

### Intermediate 82b: N-(2-((2-(dimethylamino)ethyl)(methyl)amino)-5-nitrophenyl)-2,2,2-trifluoroacetamide (81b)

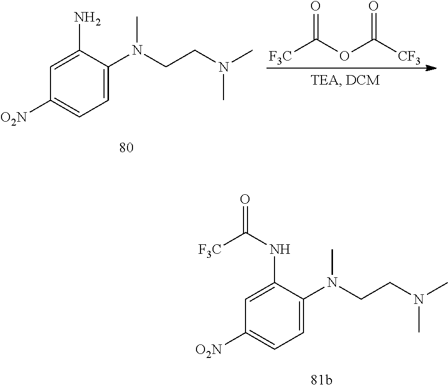

Triethylamine (3.1 g, 30.69 mmol) was added to the solution of compound 80 (3.7 g, 15.54 mmol) in dichloromethane (60 ml). The reaction mixture was cooled by ice-salt baths. Then 2,2,2-trifluoroacetic anhydride (3.92 g, 18.67 mmol) was added dropwise to the reaction mixture. The reaction mixture was stirred for 1 h at room temperature, and then acetic acid (0.4 ml) was added. The reaction mixture was stirred for 0.5 h, washed with water (50 ml*3) and then concentrated under reduced pressure to give the title compound 81b (3.9 g, yield 75.1%). MS-ESI (M+H)⁺:335.2.

### N-(5-amino-2-((2-(dimethylamino)ethyl)(methyl)amino)phenyl)-2,2,2-trifluoroacetamide (82b)

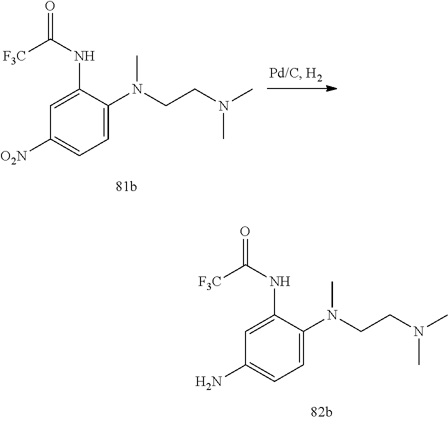 [SMILES: CN(C)CCN(C)c1ccc([N+](=O)[O-])cc1NC(=O)C(F)(F)F ; CN(C)CCN(C)c1ccc(N)cc1NC(=O)C(F)(F)F]

The compound 81b (3.9 g, 11.67 mmol) was mixed with 5% Pd/C (0.2 g, 0.09 mmol) in methanol (50 ml). The resulting mixture was stirred at r.t overnight under hydrogen atmosphere. The reaction mixture was filtered and concentrated. The residue was purified by flash column chromatography (dichloromethane/methanol=10/1) to give the title compound 82b (1.4 g, yield 39.3%). MS-ESI (M+H)⁺:305.4.

### Intermediate 82c: N-(2-((2-(dimethylamino)ethyl)(methyl)amino)-5-nitrophenyl)propionamide (81c)

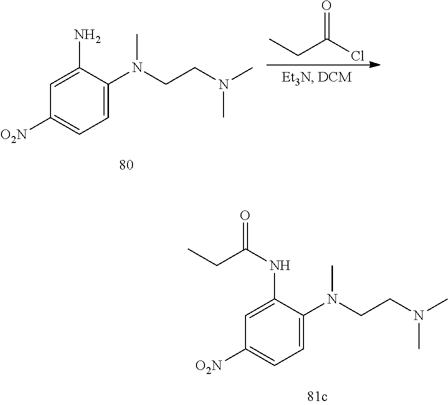

A solution of 80 (5.00 g, 20.98 mmol) and Et₃N (4.22 g, 41.78 mmol) in dichloromethane (20 mL) was stirred at 0° C. for 15 min. Then propionyl chloride (3.05 g, 32.96 mmol) was added dropwise and the mixture was stirred at 0° C. for another 2.5 h. The reaction mixture was quenched by saturated NaHCO₃ (40 mL) and diluted with dichloromethane (40 mL). The organic phase was washed with H₂O (20 mL*2). After concentration, the residue was dried in an air-circulating oven at 45° C. to give the product 81c (4.70 g, yield 76.10%) as a black oil.

### N-(5-amino-2-((2-(dimethylamino)ethyl)(methyl)amino) phenyl)propionamide (82c)

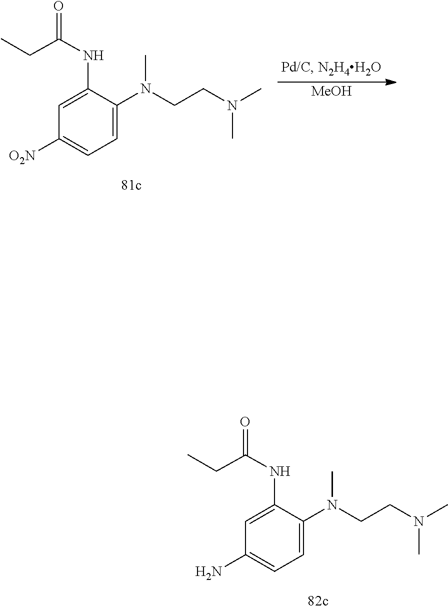 [SMILES: "MeOH" ; CCC(=O)Nc1cc([N+](=O)[O-])ccc1N(C)CCN(C)C ; CCC(=O)Nc1cc(N)ccc1N(C)CCN(C)C]

To a solution of 81c (4.70 g, 15.97 mmol) in methanol (15 mL), 5% Pd/C (0.47 g, w %=10%) and hydrazine hydrate (80%, 40 mL) were added. The mixture was stirred at 55° C. overnight. The mixture was filtered off and the filtrate was concentrated under reduced pressure. The residue diluted with dichloromethane (100 mL). The organic phase was washed with H₂O (25 mL*2), dried with Na₂SO₄. After concentration, the residue was purified by flash column chromatography (dichloromethane/methanol=20/1) to give the product 82c (1.82 g, yield 43.12%) as a light yellow oil.

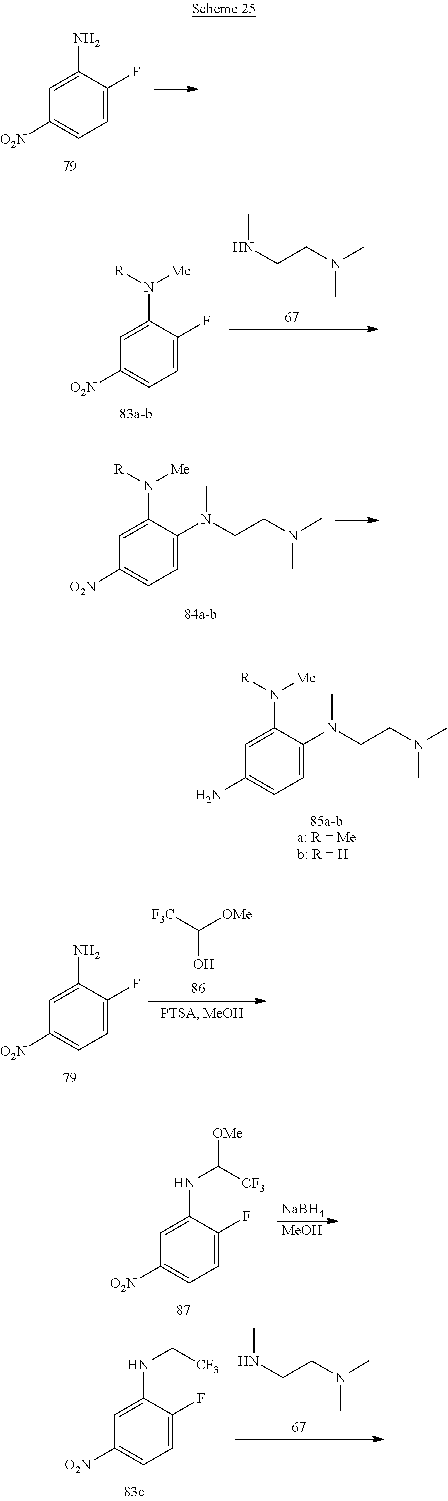

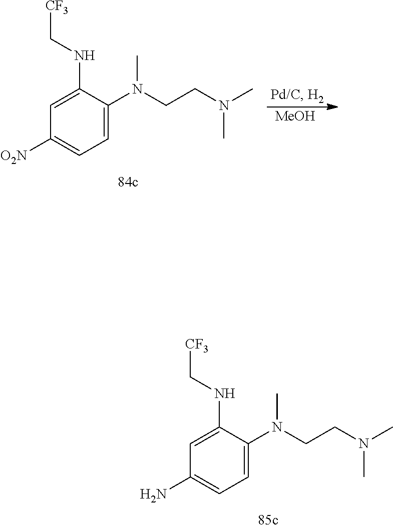

### Intermediate 85a: 2-fluoro-N,N-dimethyl-5-nitroaniline (83a)

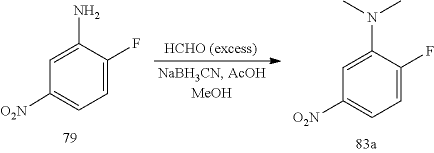

2-fluoro-5-nitro-aniline 79 (1.041 g, 6.7 mmol) was dissolved in methanol (50 ml) and 33% aqueous formaldehyde solution (5 ml, 57.1 mmol) was added at 0° C. After the addition of NaBH₃CN (2.165 g, 35.1 mmol) and acetic acid (7.5 ml), the reaction mixture was stirred at room temperature for 22 h. The reaction was quenched with H₂O (20 mL), and the pH was adjusted to 8˜9 with NaOH (4 g). The resulting mixture was extracted with dichloromethane (100 mL). The organic phase was separated and concentrated under reduced pressure to give the crude title compound 83a (1.2 g, yield 98%) as a yellow oil, which was used in the next step directly without further purification.

### N¹-(2-(dimethylamino)ethyl)-N¹,N²,N²-trimethyl-4-nitrobenzene-1,2-diamine (84a)

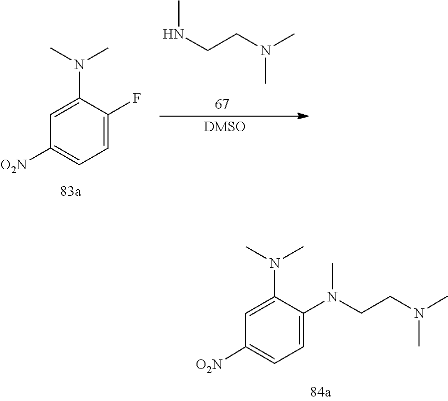

A mixture of 83a (182.6 mg, 0.99 mmol), N¹,N¹,N²-trimethylethane-1,2-diamine 67 (305.2 mg, 2.99 mmol) in DMSO (1 mL) was stirred at 120° C. overnight. After cooling to room temperature, the mixture was diluted with ethyl acetate and washed with H₂O. The organic phase was separated and concentrated under reduced pressure. The residue was purified by flash column chromatography (dichloromethane/methanol=15/1) to give the product 84a (0.23 g, yield 87.1%) as a deep red oil.

### N¹-(2-(dimethylamino)ethyl)-N¹,N²,N²-trimethylbenzene-1,2,4-triamine (85a)

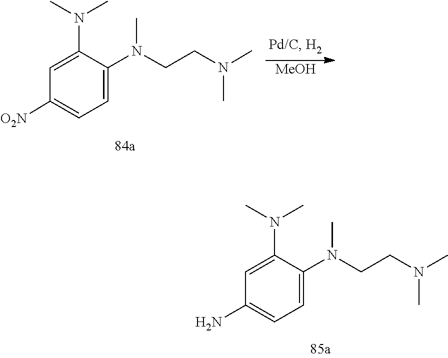 [SMILES: "MeOH" ; CN(C)CCN(C)c1ccc([N+](=O)[O-])cc1N(C)C ; CN(C)CCN(C)c1ccc(N)cc1N(C)C]

A solution of 84a (0.25 g, 0.94 mmol) and 5% Pd/C (105.3 mg, 0.05 mmol) in methanol (10 mL) was hydrogenated with hydrogen balloon at room temperature for 6.5 h. After completion of the reaction, the reaction mixture was filtered through Celite®. The filtrate was concentrated to afford the crude compound 85a (0.15 g, yield 91%) as a dark oil, which was used in the next step directly without further purification.

### Intermediate 85b: 2-fluoro-N-methyl-5-nitroaniline (83b)

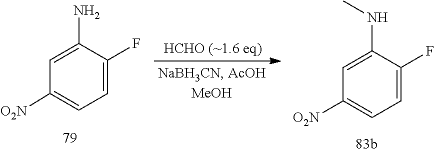

2-fluoro-5-nitro-aniline 79 (1.5947 g, 10.2 mmol) was dissolved in methanol (30 ml) and 33% aqueous formaldehyde solution (1.5 ml, 17.1 mmol) was added at 0° C. After the addition of NaBH₃CN (1.2004 g, 19.4 mmol) and acetic acid (1 ml), the reaction mixture was stirred at room temperature for 19 h. The reaction was quenched with H₂O (10 mL), and the pH was adjusted to 8-9 with NaOH (0.5 g). The desired product 83b was precipitated from the solution as a yellow solid and obtained by filtration (1.2 g, yield 69.0%).

### N¹-(2-(dimethylamino)ethyl)-N¹,N²-dimethyl-4-nitrobenzene-1,2-diamine (84b)

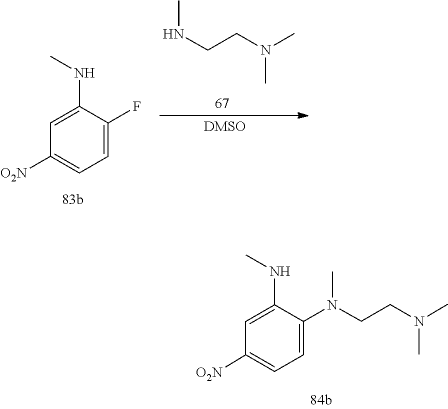

Synthesis of 84b was similar to that of 84a by using 83b (1.558 g, 9.15 mmol) and N¹,N¹,N²-trimethylethane-1,2-diamine 67 (3.0565 g, 29.9 mmol). The crude product 84b (2.378 g) was used directly in the next step without further purification.

### N¹-(2-(dimethylamino)ethyl)-N¹,N²-dimethylbenzene-1,2,4-triamine (85b)

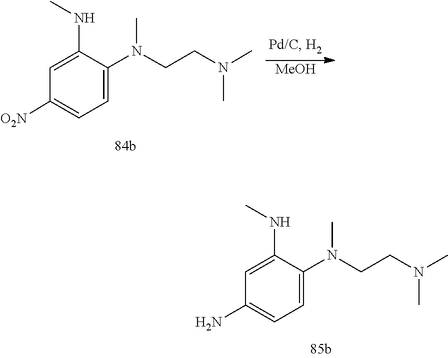 [SMILES: "MeOH" ; CNc1cc([N+](=O)[O-])ccc1N(C)CCN(C)C ; CNc1cc(N)ccc1N(C)CCN(C)C]

Synthesis of 85b was similar to that of 85a by using 84b (2.378 g, 9.4 mmol) and 5% Pd/C (0.993 g, 0.47 mmol). The crude product 85b (2.0 g, yield 95.2%) was used directly in the next step without further purification.

### Intermediate 85c: 2-fluoro-5-nitro-N-(2,2,2-trifluoro-1-methoxyethyl)aniline (87)

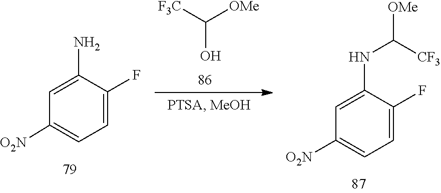 [SMILES: Nc1cc([N+](=O)[O-])ccc1F ; COC(O)C(F)(F)F ; COC(Nc1cc([N+](=O)[O-])ccc1F)C(F)(F)F]

Trifluoroacetaldehyde methyl hemiacetal 86 (412 mg, 3.2 mmol), the primary aniline 79 (156.3 mg, 1.0 mmol) and p-toluenesulfonic acid monohydrate (26.2 mg, 0.14 mmol) were dissolved in methanol (5 mL). The reaction mixture was stirred at refluxing temperature for 24 h. After cooling to room temperature, the contents of the flask were concentrated to remove the methanol, and then diluted with ethyl acetate and aqueous NaHCO₃. The aqueous layer was separated and extracted with ethyl acetate. The combined organic layers were concentrated under reduced pressure. The crude was purified by flash column chromatography (eluted with petroleum ether/ethyl acetate=15/1) to give the product 87 (220 mg, yield 82.0%) as a yellow solid.

### 2-fluoro-5-nitro-N-(2,2,2-trifluoroethyl)aniline (83c)

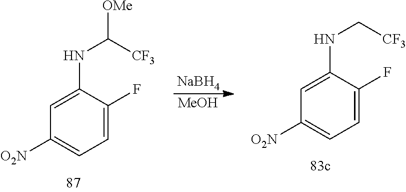

The compound 87 (220 mg, 0.82 mmol) and NaBH₄ (108.6 mg, 2.9 mmol) were dissolved in methanol (5 mL). The reaction mixture was stirred at 60° C. for 2 h. The reaction mixture was concentrated to remove the methanol, and then quenched with H₂O. The aqueous layer was extracted with ethyl acetate. The combined organic layers were concentrated under reduced pressure to give the crude product 83c (170 mg, yield 87%) without further purification.

### N¹-(2-(dimethylamino)ethyl)-N¹-methyl-4-nitro-N²-(2,2,2-trifluoroethyl)benzene-1,2-diamine (84c)

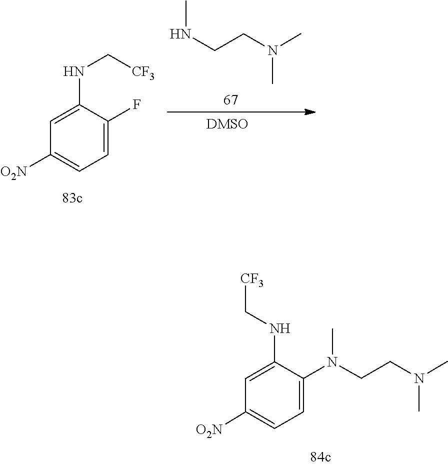

Synthesis of 84c was similar to that of 84a by using 83c (170 mg, 0.71 mmol) and N¹,N¹,N²-trimethylethane-1,2-diamine 67 (228.7 mg, 2.23 mmol). The crude product 84c (330 mg) was used directly in the next step without further purification.

### N¹-(2-(dimethylamino)ethyl)-N¹-methyl-N²-(2,2,2-trifluoroethyl)benzene-1,2,4-triamine (85c)

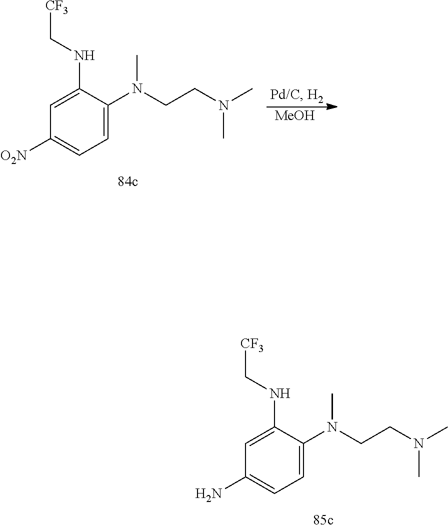

Synthesis of 85c was similar to that of 85a by using 84c (330 mg, 1.0 mmol) and 5% Pd/C (117 mg, 0.05 mmol). The crude product 85c (250 mg) was used directly in the next step without further purification.

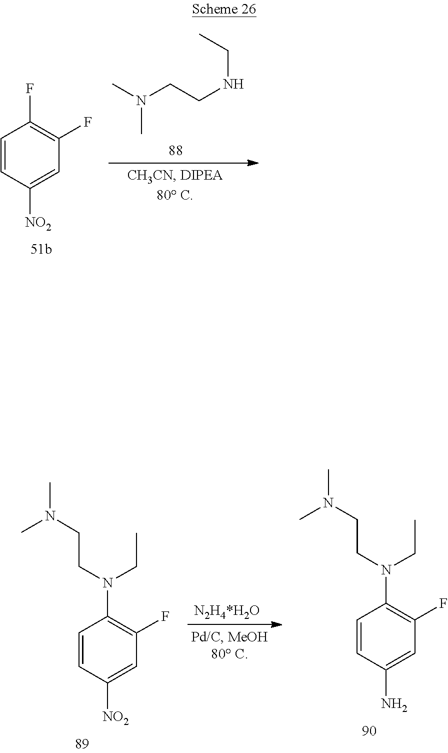

### Intermediate 90: N¹-ethyl-N¹-(2-fluoro-4-nitrophenyl)-N²,N²-dimethylethane-1,2-diamine (89)

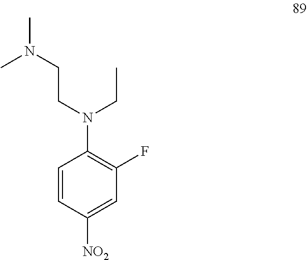 [SMILES: CCN(CCN(C)C)c1ccc([N+](=O)[O-])cc1F]

A mixture of 1,2-difluoro-4-nitrobenzene 51b (1.66 g, 10.4 mmol), N′-ethyl-N,N-dimethyl ethylenediamine 88 (1.28 g, 11.0 mmol) in CH₃CN (20 ml) was heated at 80° C. for 18 h. Then additional N′-ethyl-N,N-dimethyl ethylenediamine (0.3 g, 1.9 mmol) and N,N-diisopropyl ethylamine (1 ml, 6.1 mmol) were added to the reaction, and the mixture was stirred at 80° C. for another 4 h. After cooling to room temperature, the reaction mixtures was extracted with ethyl acetate. The organic layer was separated, washed with saturated NaHCO₃ aqueous, and concentrated to dryness. The crude was purified by flash column chromatography (eluting with ethanol) to give the title compound 89 (1.5 g, yield 56.4%) as a brown oil.

### N¹-(2-(dimethylamino)ethyl)-N¹-ethyl-2-fluorobenzene-1,4-diamine (90)

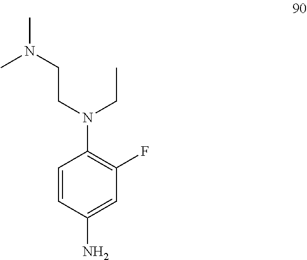 [SMILES: CCN(CCN(C)C)c1ccc(N)cc1F]

A mixture of N¹-ethyl-N¹-(2-fluoro-4-nitrophenyl)-N²,N²-dimethylethane-1,2-diamine 89 (0.3 g, 1.2 mmol), Pd/C (5% on activated carbon, 0.06 g, 0.03 mmol) and hydrazine hydrate (80%, 1 ml, 16 mmol) in methanol (10 ml) was heated at 80° C. for 2.5 h. After cooling to room temperature, the reaction mixtures was filtered through Celite® and the filtrates were concentrated to remove methanol. The residual was extracted with ethyl acetate. The organic layer was separated, washed with water, dried over Na₂SO₄, filtered and concentrated to dryness. The crude 90 (0.22 g, yield 83.0%) was directly used in the next step without further purification.

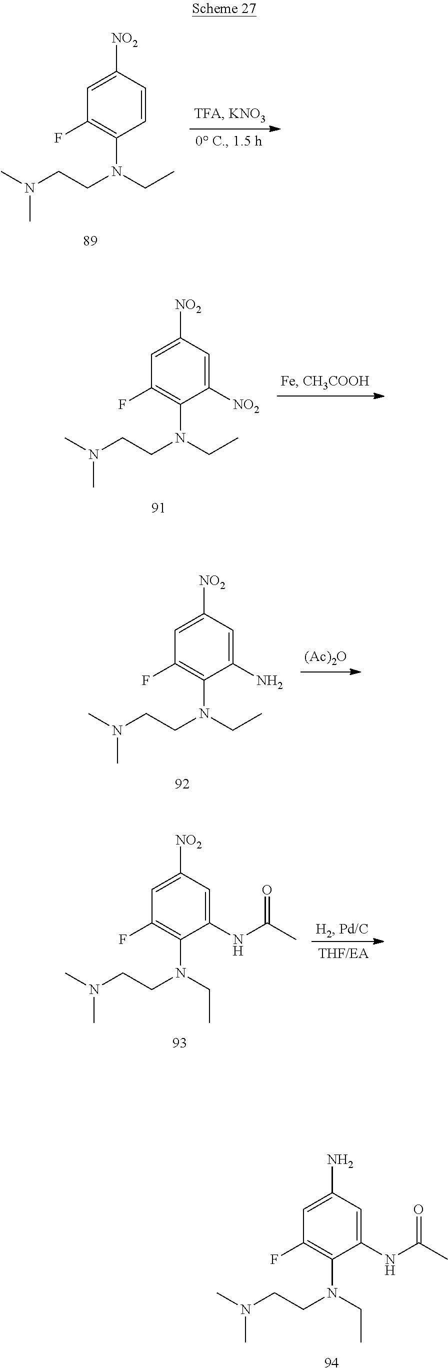

### Intermediate 94: N¹-ethyl-N¹-(2-fluoro-4,6-dinitrophenyl)-N²,N²-dimethylethane-1,2-diamine NO₂

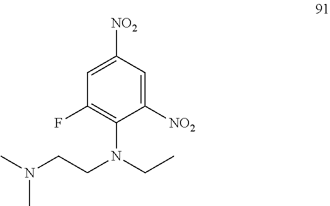 [SMILES: CCN(CCN(C)C)c1c(F)cc([N+](=O)[O-])cc1[N+](=O)[O-]]

A solution of 89 (10.0 g, 39.2 mmol) in TFA (100 mL) was stirred at 0° C. for 10 min. Then KNO₃ (7.9 g, 78.4 mmol) was added and the mixture was stirred at 0° C. for another 1.5 h. The reaction mixture was diluted with dichloromethane (100 mL) and concentrated. The residue was purified by flash column chromatography (dichloromethane to dichloromethane/methanol=50/1) to give 91 as a yellow solid as TFA salt (7.0 g, yield 43%). MS-ESI (M+H)⁺: 301.2. ¹H NMR (400 MHz, DMSO-d6) δ 8.63 (s, 1H), 8.51-8.48 (m, 1H), 3.52-3.49 (m, 2H), 3.27-3.20 (m, 4H), 2.70 (s, 6H), 1.04 (t, J=7.6 Hz, 7.6 Hz, 3H).

### N¹-(2-(dimethylamino)ethyl)-N¹-ethyl-6-fluoro-4-nitrobenzene-1,2-diamine (92)

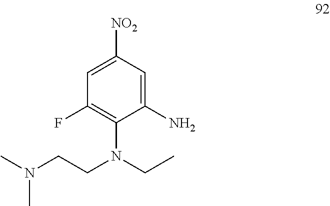 [SMILES: CCN(CCN(C)C)c1c(N)cc([N+](=O)[O-])cc1F]

To a solution of 91 (7.0 g, 23.3 mmol) in acetic acid (150 mL), iron powder (2.61 g, 46.7 mmol) was added. The mixture was stirred at room temperature for overnight. After concentration, the residue was purified by flash column chromatography (dichloromethane/methanol=100/1-15/1) to give 92 as a yellow solid (4.0 g, yield 63.5%). MS-ESI (M+H)⁺: 271.2. ¹H NMR (400 MHz, DMSO-d6) δ 7.38 (s, 1H), 7.18 (d, J=7.2 Hz, 1H), 6.16 (s, 2H), 3.26 (br, 2H), 3.02-2.99 (m, 4H), 2.61 (s, 6H), 0.93 (t, J=7.6 Hz, 3H).

### N-(2-((2-(dimethylamino)ethyl)(ethyl)amino)-3-fluoro-5-nitrophenyl)acetamide (93)

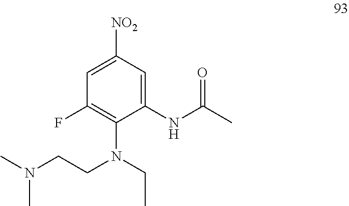 [SMILES: CCN(CCN(C)C)c1c(F)cc([N+](=O)[O-])cc1NC(C)=O]

A solution of 92 (4.0 g, 14.8 mmol) in Ac₂O (20 mL) was stirred at room temperature for overnight. The mixture was concentrated under vacuum to remove acetic anhydride and the residue was triturated with Et₂O (20 mL). The solid was collected by filtration and dried under vacuum to give 93 as a yellow solid (3.8 g, yield 82%). MS-ESI (M+H)⁺: 313.1. ¹H NMR (400 MHz, DMSO-d6) δ 9.68 (s, 1H), 9.01 (s, 1H), 7.85 (d, J=7.2 Hz, 1H), 3.44-3.42 (m, 2H), 3.10-3.68 (m, 4H), 3.08 (s, 6H), 2.24 (s, 3H), 0.89 (t, J=7.6 Hz, 3H).

### N-(5-amino-2-((2-(dimethylamino)ethyl)(ethyl)amino)-3-fluorophenyl)acetamide (94)

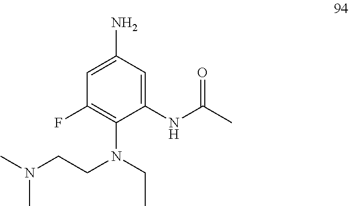 [SMILES: CCN(CCN(C)C)c1c(F)cc(N)cc1NC(C)=O]

To a solution of 93 (624 mg, 2.0 mmol) in tetrahydrofuran/ethyl acetate=1/1 (20 mL) was added Pd/C (70 mg). The mixture was stirred at room temperature for 1.5 h under hydrogen atmosphere (balloon pressure). The catalyst was filtered off and the filtrate was concentrated under vacuum to give 94 as a light brown solid (507 mg, yield 90%). MS-ESI (M+H)⁺: 283.1. ¹H NMR (400 MHz, CD₃OD) δ 7.15 (s, 1H), 6.27-6.22 (m, 1H), 3.35 (br, 2H), 3.09-3.00 (m, 4H), 2.78 (s, 6H), 2.18 (s, 3H), 0.96 (t, J=7.6 Hz, 3H).

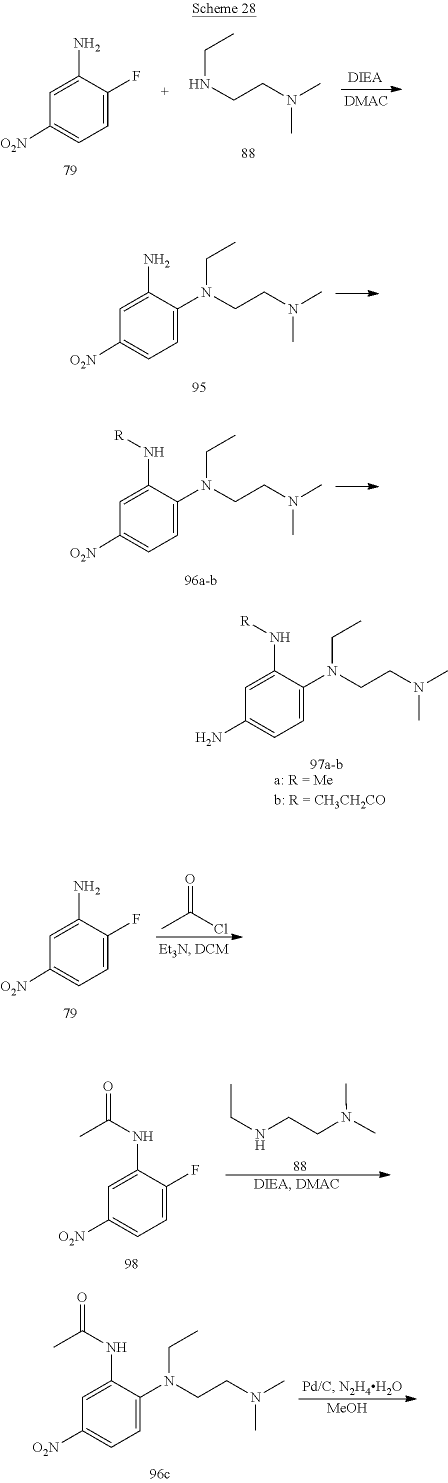

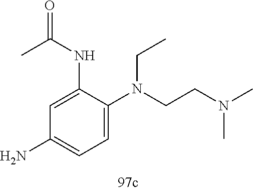 [SMILES: CCN(CCN(C)C)c1ccc(N)cc1NC(C)=O]

### Intermediate 97a: N¹-(2-(dimethylamino)ethyl)-N¹-ethyl-4-nitrobenzene-1,2-diamine (95)

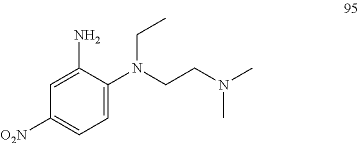 [SMILES: CCN(CCN(C)C)c1ccc([N+](=O)[O-])cc1N]

A mixture of 2-fluoro-5-nitroaniline 79 (3.00 g, 19.22 mmol), N¹-ethyl-N²,N²-dimethylethane-1,2-diamine 88 (4.61 g, 39.67 mmol) and N,N-diisopropyl ethylamine (3.62 g, 28.01 mmol) in DMAC (16 mL) was stirred at 100° C. overnight. After cooling to room temperature, the mixture was diluted with H₂O (35 mL) and extracted with dichloromethane (50 mL*2). The organic phase was merged and washed with H₂O (30 mL*2). After concentration, the residue was purified by flash column chromatography (dichloromethane/methanol=20/1) to give the product 95 (1.01 g, yield 20.83%) as a yellow oil.

### N¹-(2-(dimethylamino)ethyl)-N¹-ethyl-N²-methyl-4-nitrobenzene-1,2-diamine (96a)

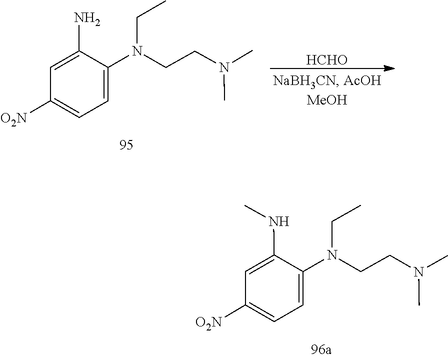

The compound 95 (0.97 g, 3.84 mmol) was dissolved in methanol (15 ml) and 33% aqueous formaldehyde solution (0.4 ml, 4.56 mmol) was added at 0° C. After the addition of NaBH₃CN (0.996 g, 15.9 mmol) and acetic acid (0.5 ml), the reaction mixture was stirred at room temperature for 19.5 h. The reaction was quenched with H₂O (1 mL), and the pH was adjusted to 8˜9 with NaOH (0.3 g). The resulting mixture was extracted with dichloromethane. The organic layers were separated and concentrated to dryness. The residue was purified by flash column chromatography (methanol/dichloromethane=1/20) to afford the title compound 96a (0.54 g, yield 52.7%) as a yellow oil.

### N¹-(2-(dimethylamino)ethyl)-N¹-ethyl-N²-methylbenzene-1,2,4-triamine (97a)

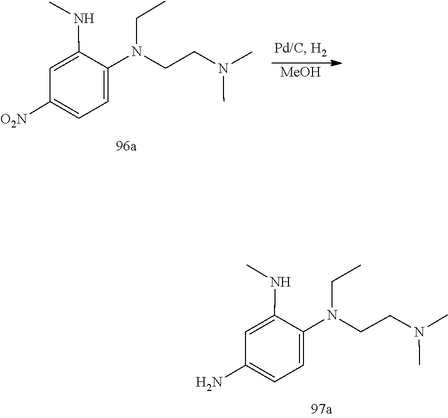 [SMILES: "MeOH" ; CCN(CCN(C)C)c1ccc([N+](=O)[O-])cc1NC ; CCN(CCN(C)C)c1ccc(N)cc1NC]

Synthesis of 97a was similar to that of 85a by using 96a (0.54 g, 2.0 mmol) and 5% Pd/C (0.21 g, 0.1 mmol). The crude product 97a (0.36 g, yield 75.1%) was used directly in the next step without further purification.

### Intermediate 97b: N-(2-((2-(dimethylamino)ethyl)(ethyl)amino)-5-nitrophenyl)propionamide (96b)

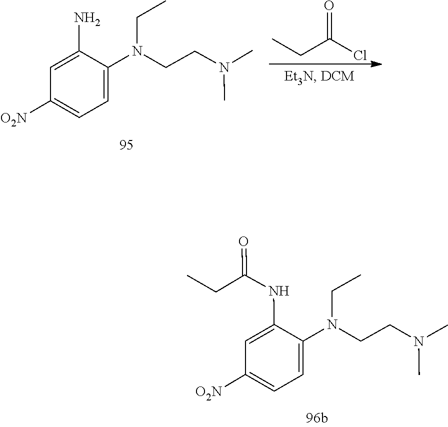

A solution of 95 (1.00 g, 3.96 mmol) and Et₃N (0.86 g, 8.51 mmol) in dichloromethane (15 mL) was stirred at 0° C. for 18 min. Then propionyl chloride (0.72 g, 7.78 mmol) was added dropwise and the mixture was stirred at 0° C. for another 1 h. The reaction mixture was quenched by saturated NaHCO₃ (10 mL) and diluted with dichloromethane (40 mL). The organic phase was washed with H₂O (20 mL*2). After concentration, the residue was dried in an air-circulating oven at 45° C. to give the product 96b (1.01 g, yield 82.64%) as a light yellow oil.

### N-(5-amino-2-((2-(dimethylamino)ethyl)(ethyl)amino) phenyl)propionamide (97b)

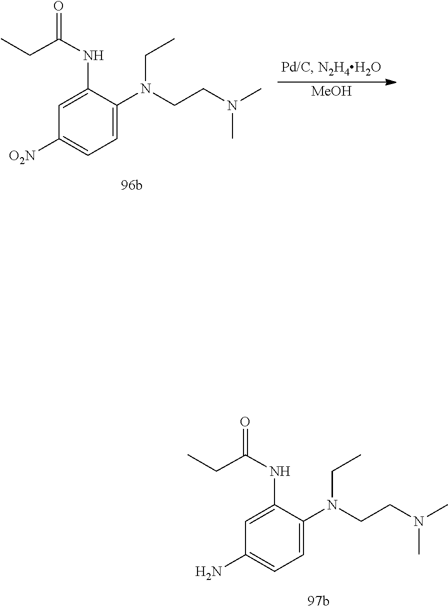 [SMILES: "MeOH" ; CCC(=O)Nc1cc([N+](=O)[O-])ccc1N(CC)CCN(C)C ; CCC(=O)Nc1cc(N)ccc1N(CC)CCN(C)C]

To a solution of 96b (1.00 g, 3.24 mmol) in methanol (10 mL), 5% Pd/C (0.1 g, w %=10%) and hydrazine hydrate (80%, 14 mL) were added. The mixture was stirred at 55° C. overnight. The mixture was filtered off and the filtrate was concentrated under reduced pressure. The residue diluted with dichloromethane (40 mL). The organic phase was washed with H₂O (15 mL*2), dried with Na₂SO₄. After concentration, the residue was purified by flash column chromatography (dichloromethane/methanol=20/1) to give the product 97b (0.68 g, yield 75.32%) as a light yellow oil.

### Intermediate 97c: N-(2-fluoro-5-nitrophenyl)acetamide (98)

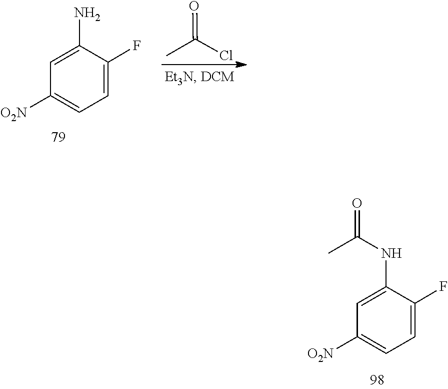

A solution of 2-fluoro-5-nitroaniline 79 (3.04 g, 19.47 mmol) and Et₃N (3.92 g, 38.81 mmol) in dichloromethane (60 mL) was stirred at 0° C. for 15 min. Then acetyl chloride (3.04 g, 38.73 mmol) was added dropwise and the mixture was stirred at 0° C. for another 2 h. The reaction mixture was quenched by saturated NaHCO₃ (20 mL) and diluted with dichloromethane (100 mL). The organic phase was washed with H₂O (20 mL*2). After concentration, the residue was purified by flash column chromatography (petroleum ether/ethyl acetate=10/1) to give the product 98 (2.67 g, yield 82.64%) as a yellow solid.

### N-(2-((2-(dimethylamino)ethyl)(ethyl)amino)-5-nitrophenyl)acetamide (96c)

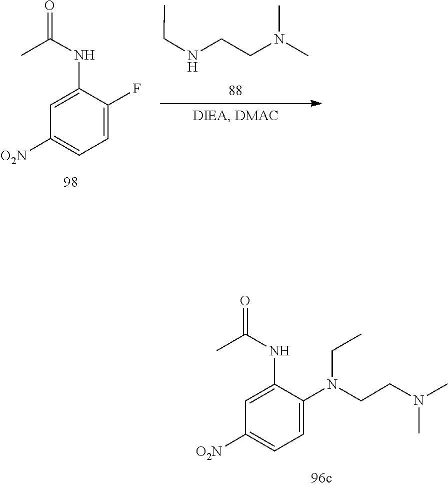

A mixture of 98 (2.50 g, 12.62 mmol), N¹-ethyl-N²,N²-dimethylethane-1,2-diamine 88 (2.81 g, 24.18 mmol) and N,N-diisopropyl ethylamine (3.02 g, 23.37 mmol) in DMAC (40 mL) was stirred at 100° C. overnight. After cooling to room temperature, the mixture was diluted with H₂O (50 mL) and extracted with ethyl acetate (60 mL*3). The organic phase was merged and washed with H₂O (30 mL*3). After concentration, the residue was purified by flash column chromatography (dichloromethane/methanol=20/1) to give the product 96c (2.6 g, yield 70.01%) as a yellow oil.

### N-(5-amino-2-((2-(dimethylamino)ethyl)(ethyl)amino)phenyl)acetamide (97c)

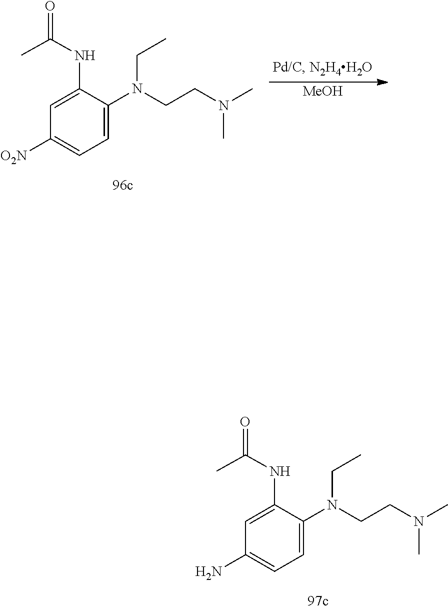 [SMILES: "MeOH" ; CCN(CCN(C)C)c1ccc([N+](=O)[O-])cc1NC(C)=O ; CCN(CCN(C)C)c1ccc(N)cc1NC(C)=O]

To a solution of 96c (2.50 g, 8.49 mmol) in methanol (25 mL), 5% Pd/C (0.28 g, w %=11%) and hydrazine hydrate (80%, 20 mL) were added. The mixture was stirred at 60° C. for 2.5 h. The mixture was filtered off and the filtrate was concentrated under reduced pressure. The residue diluted with ethyl acetate (150 mL). The organic phase was washed with H₂O (30 mL*3), dried with Na₂SO₄. After concentration, the residue was dried in an air-circulating oven at 45° C. to give the product 97c (1.86 g, yield 82.84%) as a light yellow oil.

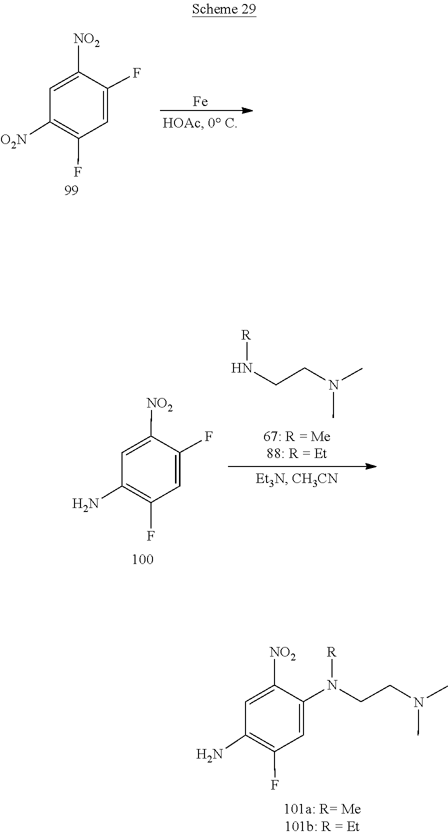

### Intermediate 101a: 2,4-difluoro-5-nitroaniline (100)

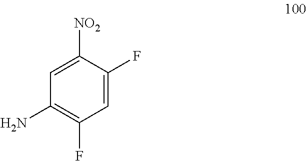 [SMILES: Nc1cc([N+](=O)[O-])c(F)cc1F]

To a solution of 99 (2.065 g, 10.1 mmol) in acetic acid (10 mL), iron powder (2.769 g, 155.1 mmol) was added. The mixture was stirred at 0° C. for 8 h followed by adding dichloromethane (30 mL). The resulting mixture was filtered through Celite® and the filter cake was washed with dichloromethane and methanol. The filtrates were concentrated in vacuo to give the crude compound 100 as a brown oil (3.18 g) without further purification.

### N¹-(2-(dimethylamino)ethyl)-5-fluoro-N¹-methyl-2-nitrobenzene-1,4-diamine (101a)

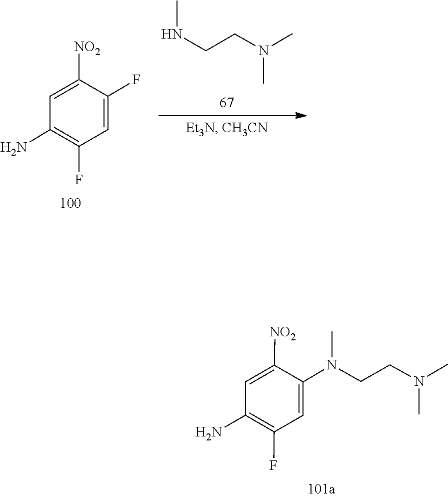

N,N,N′-trimethyl ethylenediamine 67 (1.045 g, 10.2 mmol) was added to a solution of compound 100 (1.351 g, 7.8 mmol) and triethylamine (10 mL) in CH₃CN (30 mL). The reaction mixture was stirred at 50° C. for 40 h. Then solvents were removed in vacuo and the residue was purified by flash column chromatography (dichloromethane/methanol=50/1) to give the product 101a (597 mg, yield 30%) as a red oil.

### Intermediate 101b: N¹-(2-(dimethylamino)ethyl)-N¹-ethyl-5-fluoro-2-nitrobenzene-1,4-diamine (101b)

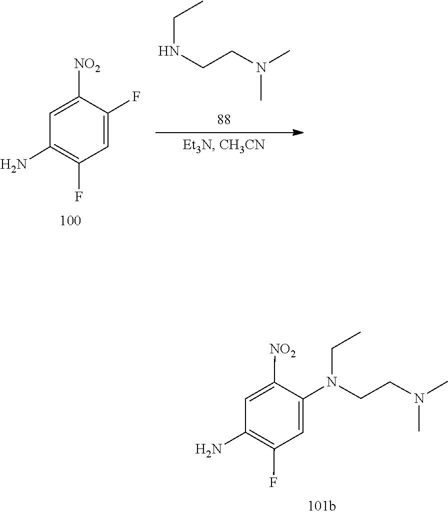

Synthesis of 101b was similar to that of 101a by using compound 100 (1.084 g, 6.2 mmol) and N¹-ethyl-N²,N²-dimethylethane-1,2-diamine 88 (1.529 g, 13.2 mmol) to give the title compound 101b (648 mg, yield 38.6%) as a red oil.

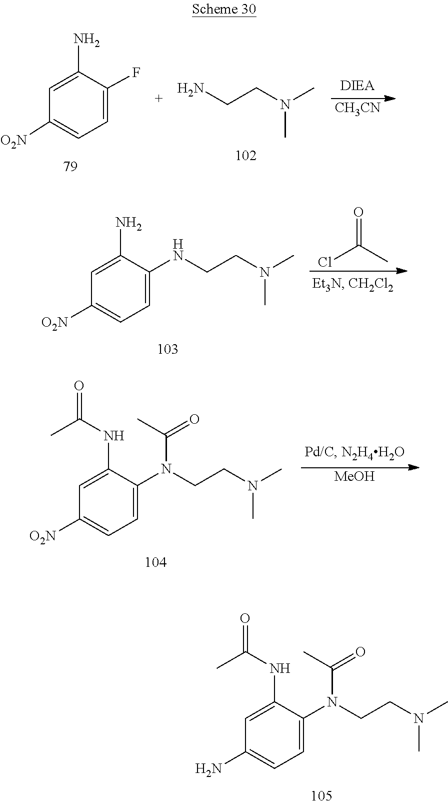

### Intermediate 105: N¹-(2-(dimethylamino)ethyl)-4-nitrobenzene-1,2-diamine (103)

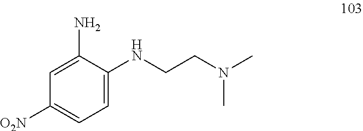 [SMILES: CN(C)CCNc1ccc([N+](=O)[O-])cc1N]

A mixture of 2-fluoro-5-nitroaniline 79 (5.09 g, 32.60 mmol), N¹,N¹-dimethylethane-1,2-diamine 102 (5.67 g, 64.32 mmol), and N,N-diisopropyl ethylamine (6.00 g, 46.15 mmol) in CH₃CN (40 mL) was stirred at 70° C. overnight. After cooling to 40° C., the mixture was concentrated under reduced pressure. The residue was dissolved in dichloromethane (200 mL), and washed with H₂O (50 mL*2), dried with Na₂SO₄. After concentration, the residue was purified by flash column chromatography (petroleum ether/ethyl acetate=2/1) to give the product 103 (3.80 g, yield 52.0%) as a reddish brown solid.

### N-(2-acetamido-4-nitrophenyl)-N-(2-(dimethylamino)ethyl) acetamide (104)

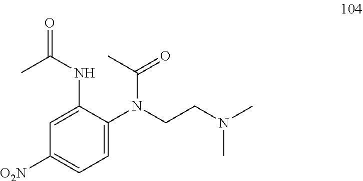 [SMILES: CC(=O)Nc1cc([N+](=O)[O-])ccc1N(CCN(C)C)C(C)=O]

A solution of 103 (3.80 g, 16.94 mmol) and Et₃N (8.55 g, 86.36 mmol) in dichloromethane (80 mL) was stirred at 0° C. for 20 min. Then acetyl chloride (5.32 g, 67.78 mmol) was added dropwise and the mixture was stirred at 0° C. for another 2.5 h. The reaction mixture was quenched by saturated NaHCO₃ (40 mL) and diluted with dichloromethane (150 mL). The organic phase was washed with H₂O (40 mL*2), then concentrated to give the product 104 (4.30 g, yield 82.30%) as a yellow solid.

### N-(2-acetamido-4-aminophenyl)-N-(2-(dimethylamino)ethyl) acetamide (105)

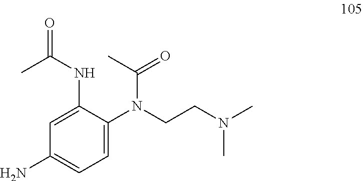 [SMILES: CC(=O)Nc1cc(N)ccc1N(CCN(C)C)C(C)=O]

To a solution of 104 (4.00 g, 12.97 mmol) in methanol (50 mL), 5% Pd/C (0.41 g, w %=10.2%) and hydrazine hydrate (80%, 40 mL) were added. The mixture was stirred at 50° C. overnight. The mixture was filtered off and the filtrate was concentrated under reduced pressure. The residue diluted with dichloromethane (100 mL). The organic phase was washed with H₂O (30 mL*2), dried with Na₂SO₄. The organic phase was concentrated to give the product 105 (3.20 g, yield 88.62%) as an off-white solid.

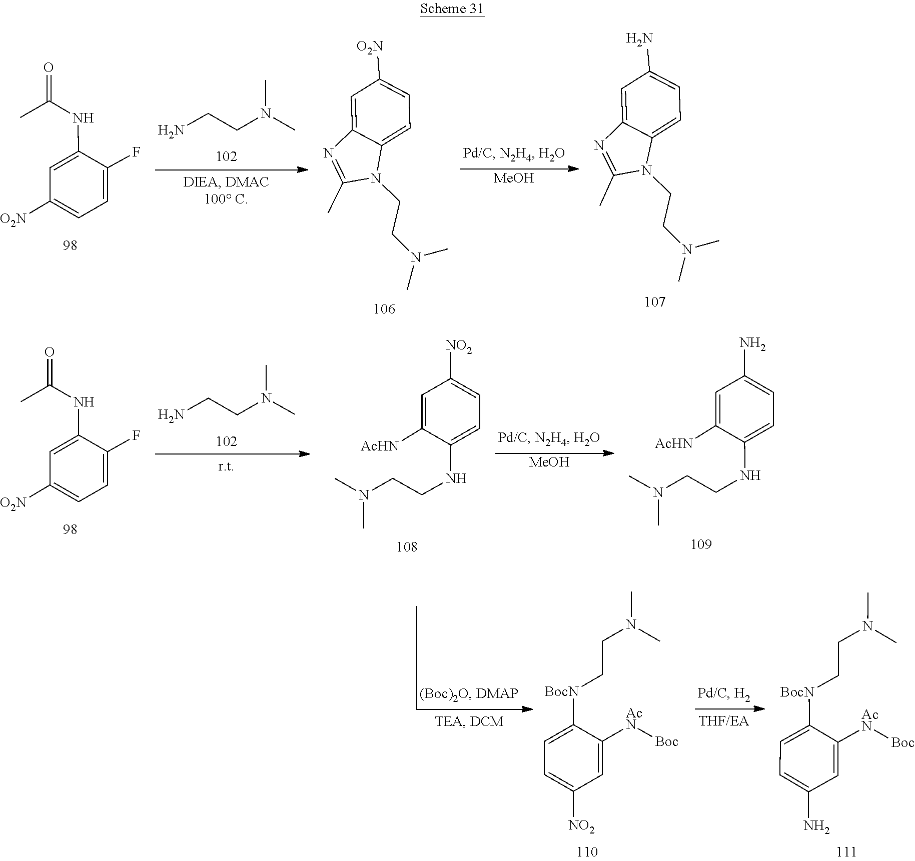

### Intermediate 107: N,N-dimethyl-2-(2-methyl-5-nitro-1H-benzo[d]imidazol-1-yl)ethanamine (106)

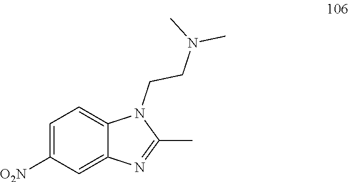 [SMILES: Cc1nc2cc([N+](=O)[O-])ccc2n1CCN(C)C]

A mixture of 98 (2.50 g, 12.62 mmol), N¹,N¹-dimethylethane-1,2-diamine 102 (2.10 g, 23.82 mmol) and N,N-diisopropyl ethylamine (3.23 g, 24.99 mmol) in DMAC (20 mL) was stirred at 100° C. overnight. After cooling to room temperature, the mixture was diluted with H₂O (40 mL) and extracted with dichloromethane (50 mL*3). The combined organic phase was washed with H₂O (30 mL*2). After concentration, the residue was dried in an air-circulating oven at 45° C. to give the product 106 (2.70 g, yield 97.50%) as a reddish-brown oil. 1-(2-(dimethylamino)ethyl)-2-methyl-1H-benzo[d]imidazol-5-amine (107)

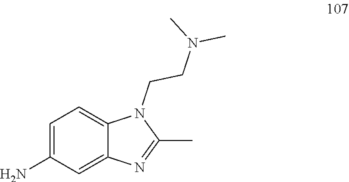 [SMILES: Cc1nc2cc(N)ccc2n1CCN(C)C]

To a solution of 106 (2.51 g, 10.11 mmol) in methanol (10 mL), 5% Pd/C (0.29 g, w %=12%) and hydrazine hydrate (80%, 20 mL) were added. The mixture was stirred at 50° C. overnight. The mixture was filtered off and the filtrate was concentrated under reduced pressure. The residue diluted with dichloromethane (100 mL). The organic phase was washed with H₂O (30 mL*2), dried with Na₂SO₄. After concentration, the residue was dried in an air-circulating oven at 45° C. to give the product 107 (1.30 g, yield 58.91%) as a red oil.

### Intermediate 109: N-(2-((2-(dimethylamino)ethyl)amino)-5-nitrophenyl)acetamide (108)

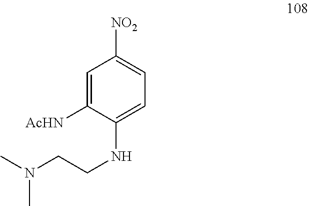 [SMILES: CC(=O)Nc1cc([N+](=O)[O-])ccc1NCCN(C)C]

A solution of 98 (1.0 g, 5.05 mmol) in 102 (3 mL) was stirred at room temperature overnight. The mixture was diluted with dichloromethane (100 mL) washed with NaCl (100 mL*2), dried with Na₂SO₄. After concentration, the crude product 108 was directly used for next step. MS-ESI (M+H)⁺: 267.1

### N-(5-amino-2-((2-(dimethylamino)ethyl)amino)phenyl)acetamide (109)

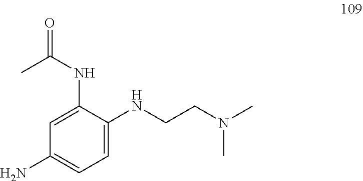 [SMILES: CC(=O)Nc1cc(N)ccc1NCCN(C)C]

To a solution of 108 (1.30 g, 1.88 mmol) in methanol (10 mL), 5% Pd/C (0.14 g, w %=11%) and hydrazine hydrate (80%, 10 mL) were added. The mixture was stirred at 50° C. overnight. The mixture was filtered off and the filtrate was concentrated under reduced pressure. The residue diluted with dichloromethane (50 mL). The organic phase was washed with H₂O (15 mL*2), dried with Na₂SO₄. After concentration, the residue was dried in an air-circulating oven at 45° C. to give the product 109 (0.67 g, yield 58.08%) as a red oil.

### Intermediate 111: tert-butyl(2-(N-(tert-butoxycarbonyl)acetamido)-4-nitrophenyl)(2-(dimethylamino)ethyl)carbamate (110)

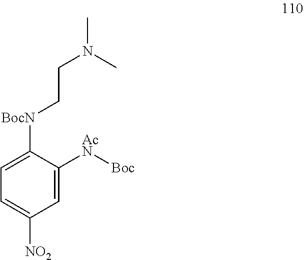

To a solution of 108 (1.2 g, 5.05 mmol) in dichloromethane (50 mL), was added Et₃N (5 mL), DMAP (616 g, 5.05 mmol) and (Boc)₂O (10 mL) and the mixture was stirred at room temperature overnight. The mixture was diluted with dichloromethane (100 mL), washed with H₂O (100 mL) and brine (100 mL), dried with Na₂SO₄. After concentration, the residue was purified by flash column chromatography (dichloromethane-dichloromethane/methanol=50/1) to give 110 as a yellow solid (1.0 g, yield 42%). ¹H NMR (400 MHz, CDCl₃) δ 8.23-8.20 (m, 1H), 8.16 (d, J=8.0 Hz, 1H), 7.55 (d, J=8.0 Hz, 1H), 3.06-2.99 (d, J=7.6 Hz, 2H), 2.41-2.34 (m, 2H), 2.13 (s, 6H), 1.87 (s, 3H), 1.50 (s, 18H). MS-ESI (M+H)⁺: 467.3.

### tert-butyl(4-amino-2-(N-(tert-butoxycarbonyl)acetamido)phenyl)(2-(dimethylamino)ethyl)carbamate (111)

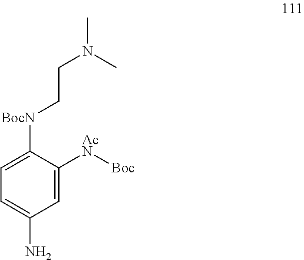

To a solution of 110 (0.75 g, 1.61 mmol) in tetrahydrofuran/ethyl acetate=1/1 (20 mL), was added Pd/C (300 mg). The mixture was stirred under H₂ at room temperature for 1.5 h. The catalyst was filtered out and the filtrate was concentrated to give title product 111 as an off-white solid (1.0 g), which was used for next step without further purification. MS-ESI (M+H)⁺: 437.3

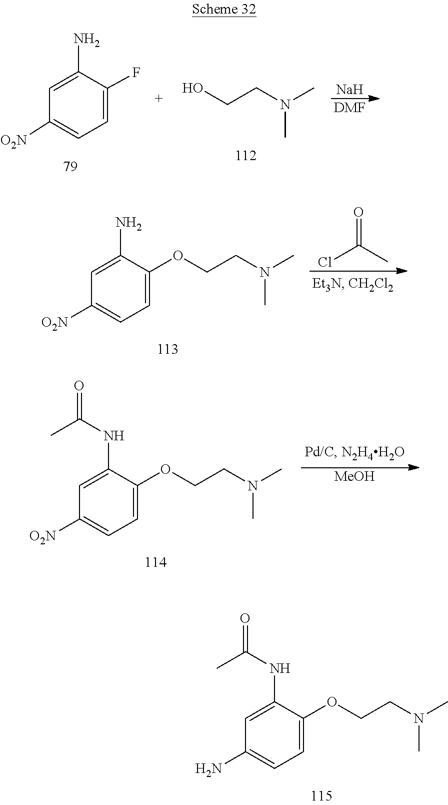

### Intermediate 115: 2-(2-(dimethylamino)ethoxy)-5-nitroaniline (113)

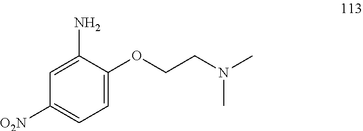 [SMILES: CN(C)CCOc1ccc([N+](=O)[O-])cc1N]

A solution of 2-(dimethylamino)ethanol 112 (8.60 g, 96.48 mmol) in DMF (100 mL) was stirred at 0° C. for 15 min. Then NaH (2.94 g, 122.51 mmol) was added in batches and the mixture was stirred at 0° C. for another 0.5 h followed by addition of 2-fluoro-5-nitroaniline 79 (5.00 g, 32.03 mmol). The mixture was stirred at room temperature for another 3.5 h. The reaction mixture was quenched by H₂O (50 mL) and diluted with ethyl acetate (200 mL). The organic phase was washed with H₂O (40 mL*2), then concentrated to give the product 113 (8.3 g, yield 115%) as a claret oil.

### N-(2-(2-(dimethylamino)ethoxy)-5-nitrophenyl)acetamide (114)

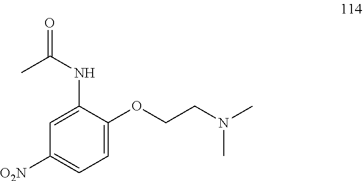 [SMILES: CC(=O)Nc1cc([N+](=O)[O-])ccc1OCCN(C)C]

A solution of 113 (7.21 g, 32.01 mmol) and Et₃N (9.74 g, 96.43 mmol) in dichloromethane (60 mL) was stirred at 0° C. for 25 min. Then acetyl chloride (3.80 g, 54.78 mmol) was added dropwise and the mixture was stirred at 0° C. for another 1.5 h. The reaction mixture was quenched by saturated NaHCO₃ (40 mL) and diluted with dichloromethane (120 mL). The organic phase was washed with H₂O (40 mL*2), then concentrated to give the product 114 (9.20 g, yield 107%) as a brownish red oil.

### N-(5-amino-2-(2-(dimethylamino)ethoxy)phenyl)acetamide (115)

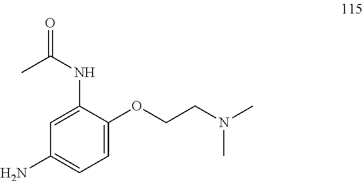 [SMILES: CC(=O)Nc1cc(N)ccc1OCCN(C)C]

To a solution of 114 (8.56 g, 32.03 mmol) in methanol (50 mL), 5% Pd/C (0.82 g, w %=9.6%) and hydrazine hydrate (80%, 80 mL) were added. The mixture was stirred at 50° C. overnight. The mixture was filtered off and the filtrate was concentrated under reduced pressure. The residue diluted with dichloromethane (150 mL). The organic phase was washed with H₂O (40 mL*2), dried with Na₂SO₄. The organic phase was concentrated to give the product 115 (5.00 g, yield 65.8%) as a black mucus.

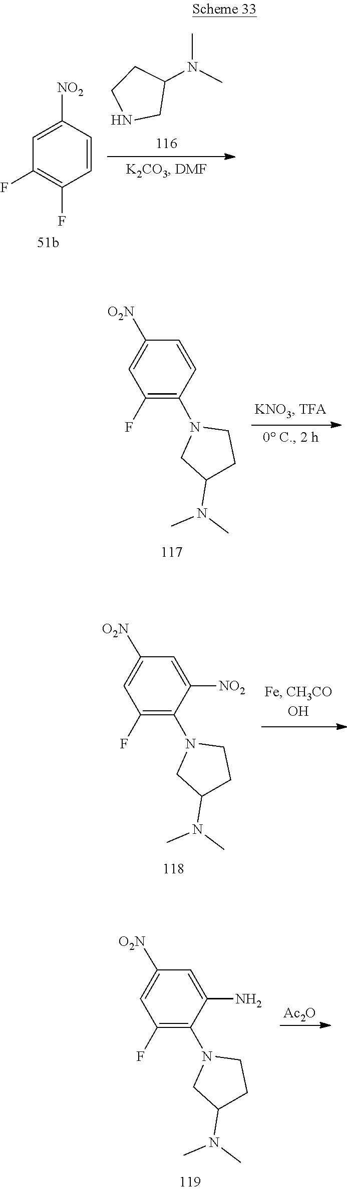

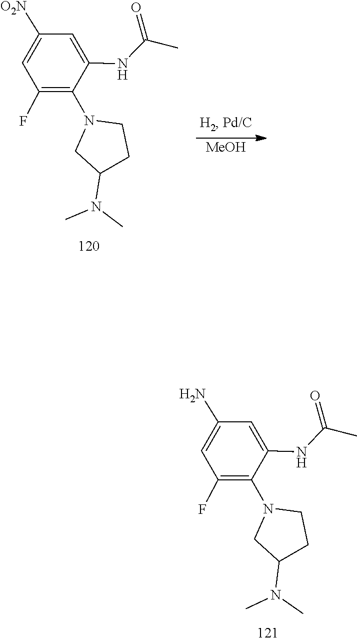 [SMILES: "MeOH" ; CC(=O)Nc1cc([N+](=O)[O-])cc(F)c1N1CCC(N(C)C)C1 ; CC(=O)Nc1cc(N)cc(F)c1N1CCC(N(C)C)C1]

### Intermediate 121: 1-(2-fluoro-4-nitrophenyl)-N,N-dimethylpyrrolidin-3-amine (117)

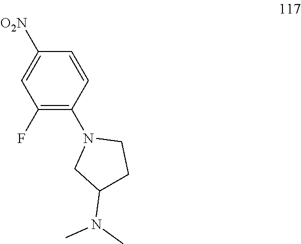 [SMILES: CN(C)C1CCN(c2ccc([N+](=O)[O-])cc2F)C1]

A mixture of 1,2-difluoro-4-nitrobenzene 51b (3 g, 18.9 mmol), N,N-dimethylpyrrolidin-3-amine 116 (2 g, 17.9 mmol) and K₂CO₃ (12 g, 94.3 mmol) in DMF (30 mL) was stirred at 110° C. for 2 h. After cooling to room temperature, the mixture was diluted with 100 mL of water. The aqueous phase was extracted with dichloromethane. The organic phase was washed with brine, dried over MgSO₄ and concentrated under reduced pressure. The resulting product 117 (4.0 g, 83.6% yield) was used to the next step without further purification. ¹H NMR (400 MHz, DMSO-d6) δ 7.96-7.85 (m, 2H), 6.77 (t, J=9.2 Hz, 1H), 3.74-3.64 (m, 2H), 3.61-3.51 (m, 1H), 3.33-3.30 (m, 1H), 2.77-2.74 (m, 1H), 2.20 (s, 6H), 2.15-2.08 (m, 1H), 1.79-1.76 (m, 1H).

### 1-(2-fluoro-4,6-dinitrophenyl)-N,N-dimethylpyrrolidin-3-amine (118)

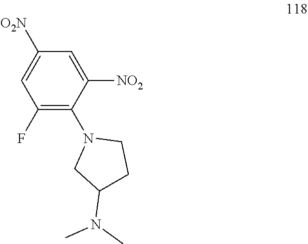 [SMILES: CN(C)C1CCN(c2c(F)cc([N+](=O)[O-])cc2[N+](=O)[O-])C1]

117 (2.5 g, 9.9 mmol) was dissolved in TFA (20 mL) and was cooled to 0° C., then KNO₃ was added and the mixture was stirred for 1 h at 0° C. The mixture was diluted with water and adjusted to pH=7 with solid NaCO₃. The aqueous phase was extracted with ethyl acetate. The organic phase was washed with brine, dried over MgSO₄ and concentrated under reduced pressure. The resulting product 118 (2.0 g, 67.8% yield) was used to the next step without further purification. m/z (MS-ESI) (M+H)⁺: 299.2.

### 1-(2-amino-6-fluoro-4-nitrophenyl)-N,N-dimethylpyrrolidin-3-amine (119)

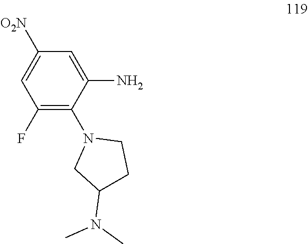 [SMILES: CN(C)C1CCN(c2c(N)cc([N+](=O)[O-])cc2F)C1]

A mixture of 118 (2.0 g, 6.7 mmol) and iron powder (1.1 g, 20.1 mmol) in acetic acid (20 mL) was stirred at room temperature for 16 h. After concentration under vacuum, the residue was triturated with ethyl acetate (200 mL) and filtered. The filtrate was t concentrated under reduced pressure to give crude title product 119, which was used to next step without further purification.

### N-(2-(3-(dimethylamino)pyrrolidin-1-yl)-3-fluoro-5-nitrophenyl)acetamide (120)

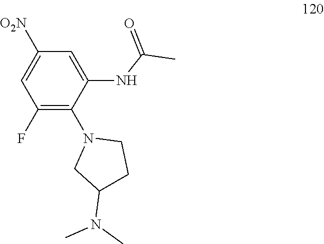 [SMILES: CC(=O)Nc1cc([N+](=O)[O-])cc(F)c1N1CCC(N(C)C)C1]

A mixture of 119 (2.0 g, 7.4 mmol) in Ac₂O (20 mL) was stirred at room temperature for 16 h. Excess solvent was removed in vacuo and the residue was purified by flash column chromatography (methanol/dichloromethane=50/1-30/1) to give the resulting product 120 (1.2 g, 52.5% yield). m/z (MS-ESI) (M+H)⁺: 311.2. ¹H NMR (400 MHz, DMSO-d6) δ 9.67 (s, 1H), 8.19 (s, 1H), 7.82 (dd, J=13.4, 2.6 Hz, 1H), 3.58-3.42 (m, 4H), 2.45 (s, 6H), 2.24-2.16 (m, 1H), 2.12 (s, 3H), 1.97-1.84 (m, 2H).

### N-(5-amino-2-(3-(dimethylamino)pyrrolidin-1-yl)-3-fluorophenyl)acetamide (121)

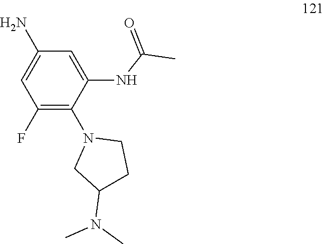 [SMILES: CC(=O)Nc1cc(N)cc(F)c1N1CCC(N(C)C)C1]

A mixture of 10% Pd/C (425 mg, 0.4 mmol) and 120 (1.2 g, 3.8 mmol) in methanol (30 mL) was stirred at room temperature for 16 h under an atmosphere of hydrogen (balloon). The catalyst was filtered off and the filtrate was concentrated under vacuum to afford title product 121 (1.06 g, 98% yield) as a purple solid, which was used to next step without further purification. m/z (MS-ESI) (M+H)⁺: 281.2.

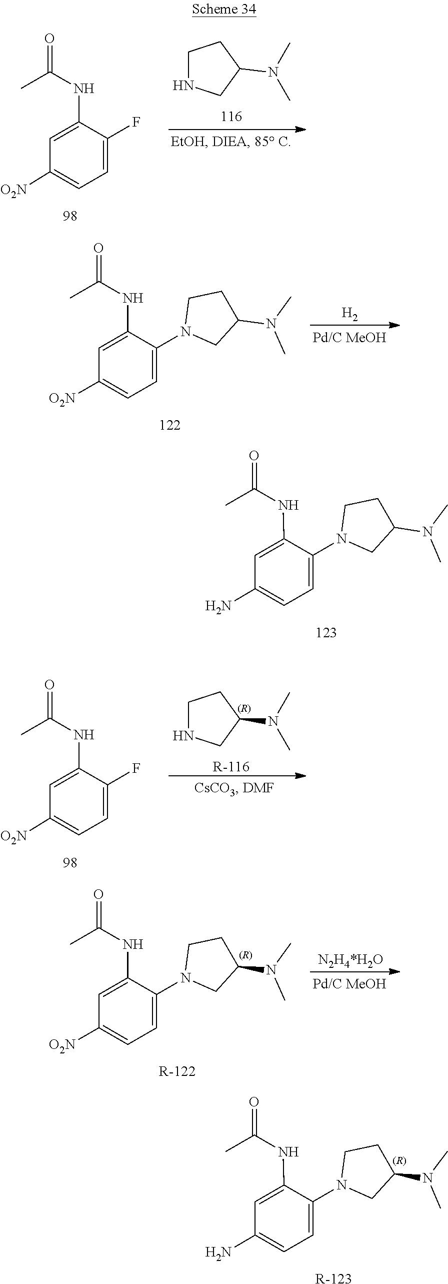

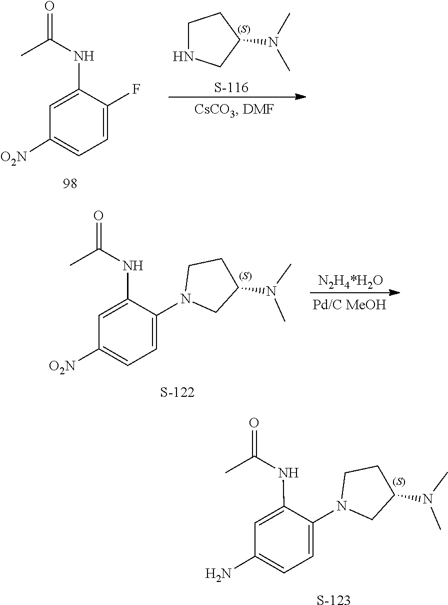

### Intermediate 123: N-(2-(3-(dimethylamino) pyrrolidin-1-yl)-5-nitrophenyl)acetamide (122)

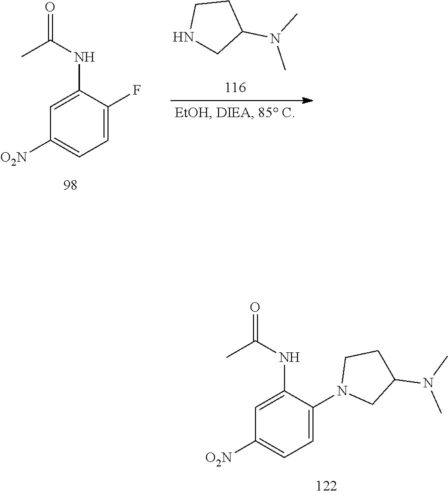

A mixture of 98 (2 g, 10.1 mmol), N,N-diisopropyl ethylamine (2.6 g, 20.2 mmol) and N,N-dimethylpyrrolidin-3-amine 116 (1.1 g, 10.1 mmol) in ethanol (30 mL) was stirred at 85° C. for 16 h. After cooling to room temperature, the mixture was concentrated and the residue was mixed with 100 ml of water and 100 ml of dichloromethane. The organic phase was separated and washed with 50 ml of brine, dried over MgSO₄ and then concentrated under reduced pressure. The resulting product 122 (3.1 g, 104% yield) was used to next step without further purification.

### N-(5-amino-2-(3-(dimethylamino) pyrrolidin-1-yl) phenyl)acetamide (123)

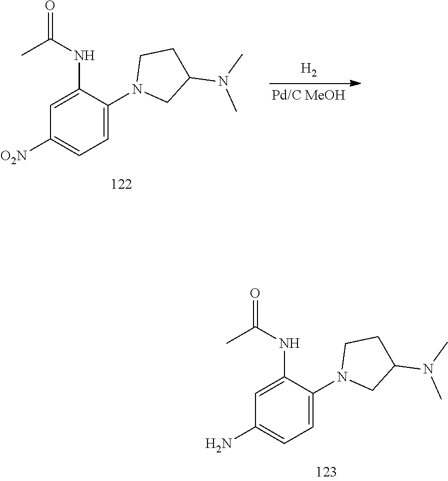 [SMILES: CC(=O)Nc1cc([N+](=O)[O-])ccc1N1CCC(N(C)C)C1 ; "H2" ; CC(=O)Nc1cc(N)ccc1N1CCC(N(C)C)C1]

A mixture of 122 (3.0 g, 10.3 mmol) and 10% Pd/C (1.1 g, 1.03 mmol) in methanol (30 mL) and allowed to stir under an atmosphere of hydrogen (balloon) for 16 h. The reaction mixture was filtered and the filtrate was then concentrated under vacuum to provide 123 (2.8 g), which was used to next step without further purification. (MS-ESI) (M+H)⁺: 263.2. ¹H NMR (400 MHz, DMSO-d6) δ 8.78 (s, 1H), 7.18 (s, 1H), 6.83 (d, J=8.5 Hz, 1H), 6.26 (d, J=6.0 Hz, 1H), 4.77 (s, 2H), 3.03 (dd, J=16.0, 7.6 Hz, 1H), 2.93-2.67 (m, 4H), 2.15 (s, 6H), 2.04-2.02 (m, 2H), 1.83-1.70 (m, 1H).

### Intermediate R-123: (R)—N-(2-(3-(dimethylamino)pyrrolidin-1-yl)-5-nitrophenyl) acetamide (R-122)

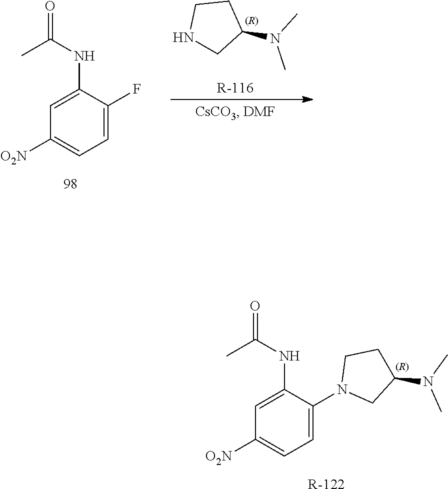

A mixture of 98 (1.8 g, 9.08 mmol), (R)—N,N-dimethylpyrrolidin-3-amine R-116 (1.00 g, 8.76 mmol), and Cs₂CO₃ (4.46 g, 13.69 mmol) in DMF (40 mL) was stirred at 80° C. for 2 h. The reaction mixture was quenched by H₂O (50 mL) and diluted with ethyl acetate (50 mL). The organic phase was washed with H₂O (40 mL*3), then concentrated to give the product R-122 (2.27 g, yield 85.5%) as a yellow solid.

### (R)—N-(5-amino-2-(3-(dimethylamino)pyrrolidin-1-yl)phenyl) acetamide (R-123)

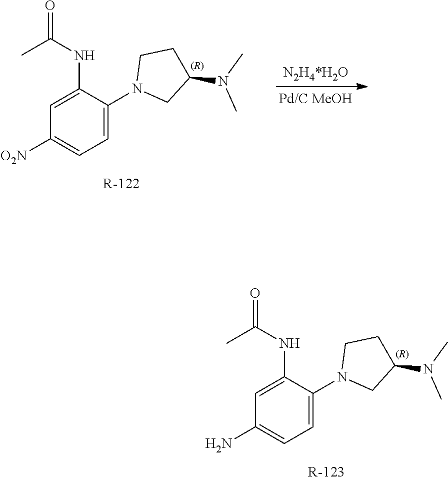

To a solution of R-122 (2.27 g, 7.77 mmol) in methanol (20 mL), 5% Pd/C (0.22 g, w %=9.7%) and hydrazine hydrate (80%, 20 mL) were added. The mixture was stirred at 60° C. overnight. The mixture was filtered off and the filtrate was concentrated under reduced pressure. The residue was diluted with dichloromethane (50 mL). The organic phase was washed with H₂O (20 mL*2), dried with Na₂SO₄. The organic phase was concentrated to give the product R-123 (1.50 g, yield 73.6%) as a dark brown oil.

### Intermediate S-123: (S)—N-(2-(3-(dimethylamino)pyrrolidin-1-yl)-5-nitrophenyl) acetamide (S-122)

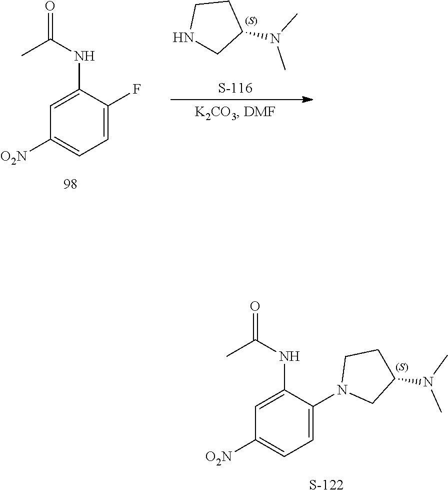

A mixture of 98 (2.0 g, 10.09 mmol), (S)—N,N-dimethylpyrrolidin-3-amine S-116 (1.22 g, 10.68 mmol), and K₂CO₃ (2.82 g, 20.43 mmol) in DMF (30 mL) was stirred at 80° C. for 2 h. The reaction mixture was quenched by H₂O (20 mL) and diluted with ethyl acetate (40 mL). The organic phase was washed with H₂O (20 mL*3), then concentrated to give the product S-122 (2.90 g, yield 98.3%) as a yellow solid.

### (S)—N-(5-amino-2-(3-(dimethylamino)pyrrolidin-1-yl)phenyl) acetamide (S-123)

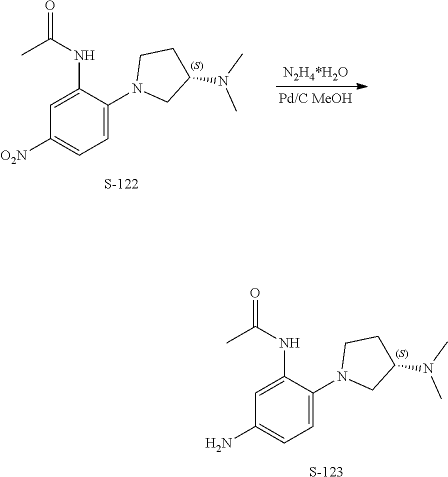

To a solution of S-122 (2.90 g, 9.92 mmol) in methanol (50 mL), 5% Pd/C (0.30 g, w %=10.3%) and hydrazine hydrate (80%, 10 mL) were added. The mixture was stirred at 50° C. overnight. The mixture was filtered off and the filtrate was concentrated under reduced pressure. The residue was diluted with dichloromethane (50 mL). The organic phase was washed with H₂O (20 mL*2), dried with Na₂SO₄. The organic phase was concentrated to give the product S-123 (2.20 g, yield 80.5%) as a light brown oil.

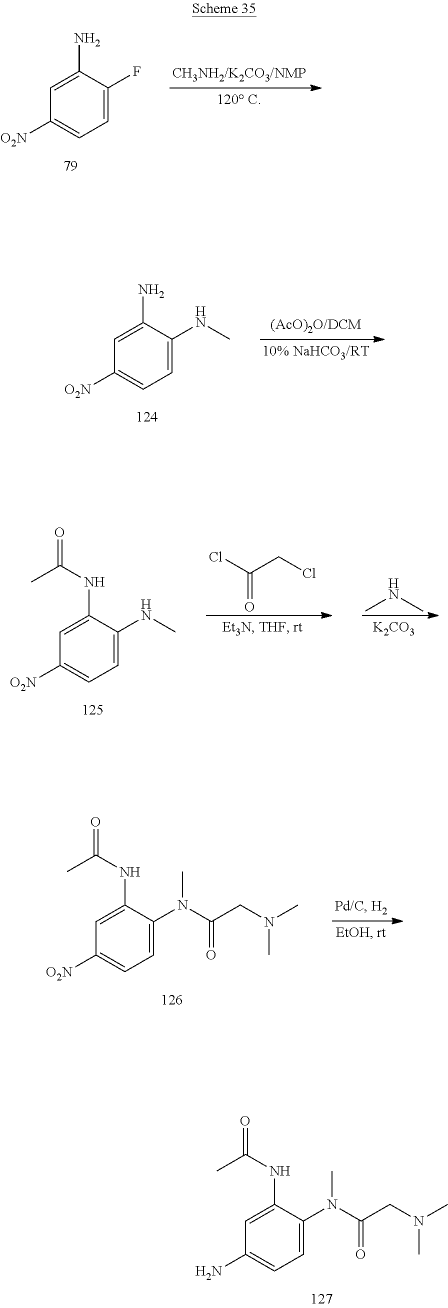

### Intermediate 127: N¹-methyl-4-nitrobenzene-1, 2-diamine (124)

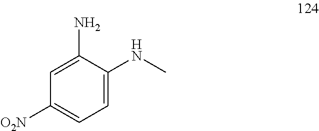

To a solution of 79 (5.0 g, 32 mmol) in 80 mL of DMSO was added Methylamine hydrochloride (4.3 g, 64 mmol), K₂CO₃ (17.7 g, 128 mmol). The mixture was stirred at 120° C. under Ar overnight. The mixture was poured into water (400 mL) to give a suspension. The suspension was filtered and the filter cake was washed with water to give a red solid. The solid was dried under reduced pressure to afford 124 (4.1 g, yield 76.6%) as a red solid. MS-ESI (M+H)⁺: 168.1. ¹H NMR (400 MHz, DMSO-d6) δ 7.55 (dd, J=8.8, 2.6 Hz, 1H), 7.39 (d, J=2.7 Hz, 1H), 6.42 (d, J=8.9 Hz, 1H), 6.11 (d, J=4.4 Hz, 1H), 5.08 (s, 2H), 2.84 (d, J=4.8 Hz, 3H).

### N-(2-(methylamino)-5-nitrophenyl)acetamide (125)

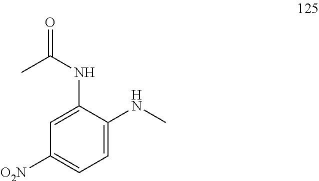

To a solution of 124 (2.0 g, 12 mmol) in 80 mL of dichloromethane and 130 mL of 10% NaHCO₃ was added acetic anhydride (5 mL). The mixture was stirred at room temperature for 3 h. The precipitate was filtered and washed with water to give a yellow solid. The solid was dried under reduced pressure to afford 125 (2.1 g, yield 84%) as a yellow solid. MS-ESI (M+H): 210.0. ¹H NMR (400 MHz, DMSO-d6) δ 9.22 (s, 1H), 8.04 (d, J=2.7 Hz, 1H), 7.98 (dd, J=9.2, 2.7 Hz, 1H), 6.65 (d, J=9.2 Hz, 2H), 2.84 (d, J=4.0 Hz, 3H), 2.06 (s, 3H).

### N-(2-acetamido-4-nitrophenyl)-2-(dimethylamino)-N-methylacetamide (126)

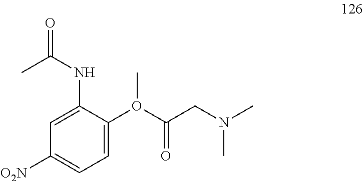

To a solution of 125 (1.0 g, 4.78 mmol) in 30 mL of tetrahydrofuran was added chloroacetyl chloride (0.8 mL, 9.57 mmol) and Et₃N (1.3 mL, 9.57 mmol). The mixture was stirred at room temperature overnight. To the mixture was added 10 mL of dimethylamine (2M solution in tetrahydrofuran) and 1.3 g of K₂CO₃, the resulting mixture was stirred at room temperature for 5 h. After this time the mixture was filtered and the filter cake was washed with dichloromethane. The combined filtrate was concentrated to give a residue. The residue was purified by flash column chromatography (dichloromethane/methanol=100/1-25/1) to afford 126 (210 mg, yield 15%) as a yellow solid. MS-ESI (M+H): 295.0. ¹H NMR (400 MHz, DMSO-d6) δ 9.73 (s, 1H), 8.92 (d, J=2.0 Hz, 1H), 8.00 (dd, J=8.7, 2.5 Hz, 1H), 7.63 (d, J=8.7 Hz, 1H), 3.06 (s, 3H), 2.75 (ABq, J=40.0, 16.0 Hz, 2H), 2.16 (s, 3H), 2.15-2.08 (m, 6H).

### N-(2-acetamido-4-aminophenyl)-2-(dimethylamino)-N-methylacetamide (127)

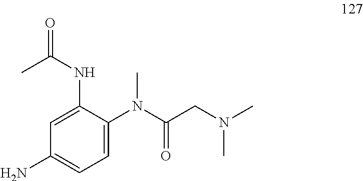 [SMILES: CC(=O)Nc1cc(N)ccc1N(C)C(=O)CN(C)C]

To a solution of 126 (210 mg, 0.71 mmol) in methanol (20 mL) was added Pd/C (30 mg). The mixture was stirred at room temperature for 3 h under H₂. The mixture was filtered over Celite® pad and the filtrate was concentrated to afford 127 (170 mg, yield 90%), which was used to next step without further purification. MS-ESI (M+H): 265.3.

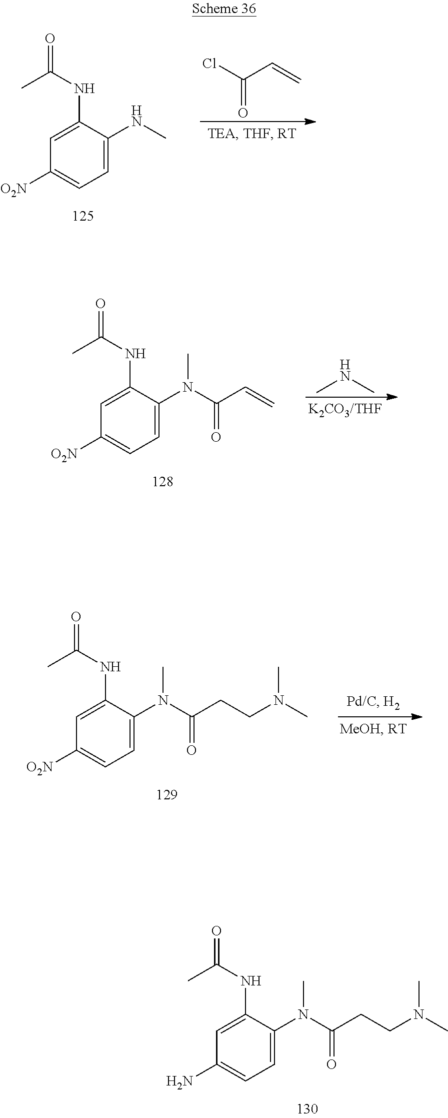

### Intermediate 130: N-(2-acetamido-4-nitrophenyl)-N-methylacrylamide (128)

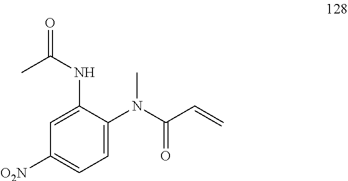 [SMILES: C=CC(=O)N(C)c1ccc([N+](=O)[O-])cc1NC(C)=O]

To a solution of 125 (1.0 g, 4.78 mmol) in 30 mL of tetrahydrofuran was added acryloyl chloride (0.8 mL, 9.57 mmol) and Et₃N (1.3 mL, 9.57 mmol). After stirring at room temperature overnight, the mixture was poured into petroleum ether (200 mL) to give a suspension. The suspension was filtered and washed with petroleum ether to give a residue. The residue was purified by flash column chromatography (petroleum ether/ethyl acetate=100/1˜1/1˜1/4) to afford 128 (1.0 g, yield 79.5%) as a yellow solid. ¹H NMR (400 MHz, CDCl₃) δ 9.41-9.25 (m, 1H), 8.03-7.98 (m, 1H), 7.92-7.81 (m, 1H), 7.32-7.28 (m, 1H), 6.46-6.38 (m, 1H), 5.93-5.83 (m, 1H), 5.69-5.60 (m, 1H), 3.34-3.24 (m, 3H), 2.31-2.23 (m, 3H).

### N-(2-acetamido-4-nitrophenyl)-N-methylacrylamide (129)

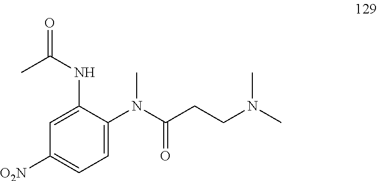 [SMILES: CC(=O)Nc1cc([N+](=O)[O-])ccc1N(C)C(=O)CCN(C)C]

8 mL of dimethylamine (2M solution in tetrahydrofuran) and 1.0 g of K₂CO₃ was placed in 30 ml of tetrahydrofuran and 1.0 g of 128 was added in three batches at room temperature. The mixture was stirred for 12 h at room temperature. After this time, the mixture was filtered and the filter cake was washed with dichloromethane to give a solution. The solution was concentrated to give a residue. The residue was purified by flash column chromatography (dichloromethane/methanol=100/1-25/1) to afford 129 (900 mg, yield 76.9%) as a yellow solid. MS-ESI (M+H): 309.2. ¹H NMR (400 MHz, CD₃OD) δ 8.96 (d, J=2.5 Hz, 1H), 8.08 (dd, J=8.7, 2.6 Hz, 1H), 7.56 (d, J=8.7 Hz, 1H), 3.34 (s, 2H), 3.18 (s, 3H), 2.78 (dt, J=12.6, 7.3 Hz, 1H), 2.47 (dt, J=12.4, 6.2 Hz, 1H), 2.23 (s, 3H), 2.13 (s, 6H).

### N-(2-acetamido-4-aminophenyl)-3-(dimethylamino)-N-methylpropanamide (130)

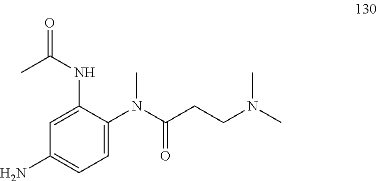 [SMILES: CC(=O)Nc1cc(N)ccc1N(C)C(=O)CCN(C)C]

To a solution of 129 (900 mg, 2.91 mmol) in methanol (20 mL) was added Pd/C (100 mg). The mixture was stirred at room temperature for 3 h under H₂. The mixture was filtered over Celite® to give a solution. The solution was concentrated to afford crude 130 (1.0 g, yield 91%), which was used to next step without further purification. MS-ESI (M+H)⁺: 279.3.

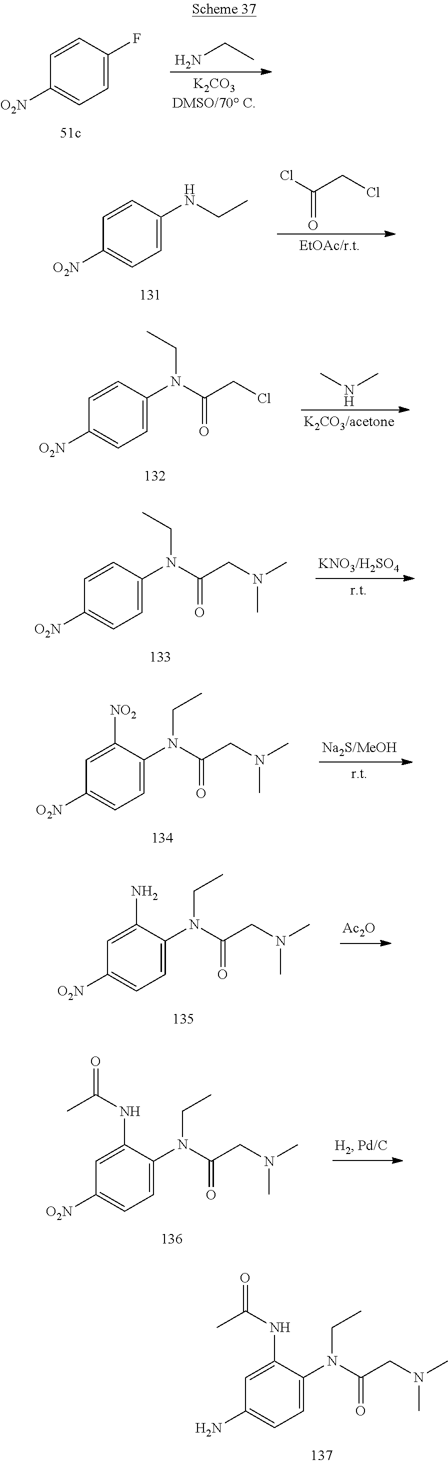

### Intermediate 137: N-ethyl-4-nitroaniline (131)

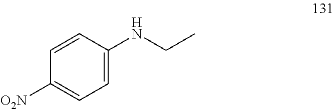

To a solution of 51c (10.0 g, 70.87 mmol) in 35 mL of DMSO was added ethylamine hydrochloride (8.7 g, 106.31 mmol) and K₂CO₃ (14.7 g, 106.31 mmol). The mixture was stirred at 70° C. under Ar overnight. The mixture was poured into water (250 mL) to give a suspension. The suspension was filtered and the filter cake was washed with water to give a yellow solid. The solid was dried under reduced pressure to afford 131 (12 g, yield 84.7%) as a yellow solid. MS-ESI (M+H)⁺: 167.2.

### 2-chloro-N-ethyl-N-(4-nitrophenyl)acetamide (132)

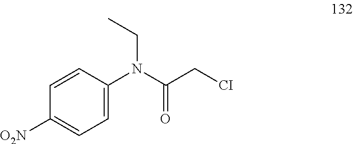

A mixture of 131 (5.0 g, 30.12 mmol) in ethyl acetate (25 mL) was heated to 60° C., chloroacetylchloride (5.1 g, 45.18 mmol) was added over 15 minutes at this temperature. The mixture was refluxed for 3 h. After cooling to room temperature, 60 mL of petroleum ether was added and the crystals were crystallized at 60° C. The mixture was cooled to 0° C. and stirred for 2 h. The precipitate was filtered, washed with petroleum ether and dried to afford 132 (6.3 g, 73% purity) as a yellow solid. MS-ESI (M+H)⁺: 243.2.

### 2-(dimethylamino)-N-ethyl-N-(4-nitrophenyl)acetamide (133)

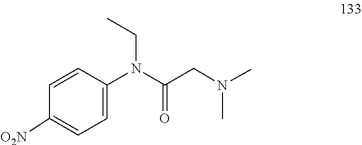 [SMILES: CCN(C(=O)CN(C)C)c1ccc([N+](=O)[O-])cc1]

19 mL of dimethylamine (2M solution in tetrahydrofuran) and 5.23 g of K₂CO₃ were placed in 80 mL of acetone, and 6.3 g of 2-chloro-N-ethyl-N-(4-nitrophenyl)acetamide 132 were added in three batches at room temperature. The mixture was stirred for 12 h at room temperature. After this time, the mixture was filtered and washed with dichloromethane to give a solution. The solution was concentrated to give a residue. The residue was purified by flash column chromatography (dichloromethane/methanol=100/1˜20/1) to afford 133 (3.7 g, yield 77.7%) as a yellow solid. MS-ESI (M+H)⁺: 252.1.

### 2-(dimethylamino)-N-(2, 4-dinitrophenyl)-N-ethylacetamide (134)

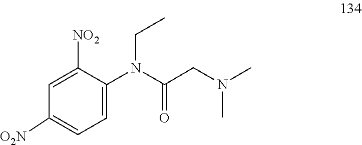 [SMILES: CCN(C(=O)CN(C)C)c1ccc([N+](=O)[O-])cc1[N+](=O)[O-]]

To a solution of 133 (3.7 g, 14.74 mmol) in H₂SO₄ (15 mL) was added KNO₃ (2.23 g, 22.11 mmol) at 0° C. The mixture was stirred at room temperature for 2 h. The mixture was adjusted to pH=7 at 0° C. with saturated Na₂CO₃ aqueous. The aqueous phase was extracted with ethyl acetate (500 mL*2). The combined organic layers were dried over anhydrous Na₂SO₄, filtered and concentrated to afford 134 (4.0 g, yield 91.7%) as a yellow solid. MS-ESI (M+H): 297.1.

### N-(2-amino-4-nitrophenyl)-2-(dimethylamino)-N-ethylacetamide (135)

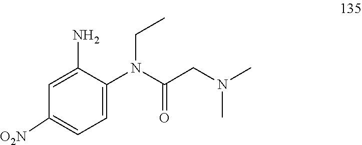 [SMILES: CCN(C(=O)CN(C)C)c1ccc([N+](=O)[O-])cc1N]

To a solution of 134 (4.0 g, 13.51 mmol) in methanol (50 mL) was added Na₂S (2.1 g, 27 mmol). The mixture was stirred at room temperature for 1 h. After concentration, the residue was purified by flash column chromatography (dichloromethane/methanol=100/1˜25/1) to afford 135 (1.7 g, yield 47.2%) as a yellow solid. MS-ESI (M+H)⁺: 267.0.

### N-(2-acetamido-4-nitrophenyl)-2-(dimethylamino)-N-ethylacetamide (136)

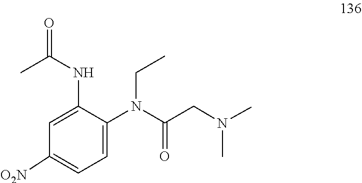 [SMILES: CCN(C(=O)CN(C)C)c1ccc([N+](=O)[O-])cc1NC(C)=O]

A solution of 135 (1.7 g, 6.4 mmol) in acetic anhydride (15 mL) was stirred at room temperature for 16 h. The mixture was concentrated in vacuo and the residue was purified by flash column chromatography (dichloromethane/methanol=100/1-25/1) to afford 136 (1.3 g, yield 66%) as a yellow solid. MS-ESI (M+H)⁺: 309.2

### N-(2-acetamido-4-aminophenyl)-2-(dimethylamino)-N-ethylacetamide (137)

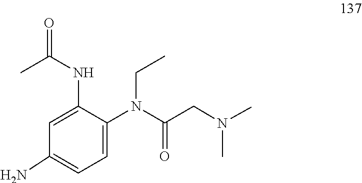 [SMILES: CCN(C(=O)CN(C)C)c1ccc(N)cc1NC(C)=O]

To a solution of 136 (4.0 g, 13.51 mmol) in methanol (50 mL) was added Pd/C (500 mg). The mixture was stirred at room temperature for 5 h under H₂. The catalyst was filtered out and the filtrate was concentrated to afford 137 (1.0 g, yield 85.5%). MS-ESI (M+H)⁺: 279.0. ¹H NMR (400 MHz, CD₃OD) δ 7.22 (d, J=2.3 Hz, 1H), 6.91 (d, J=8.5 Hz, 1H), 6.79 (dd, J=8.5, 2.3 Hz, 1H), 3.95-3.90 (m, 1H), 3.41-3.37 (m, 1H), 3.35 (d, J=16.0 Hz, 1H), 3.13 (d, J=16.1 Hz, 1H), 2.50 (s, 6H), 2.10 (s, 3H), 1.13 (t, J=7.2 Hz, 3H).

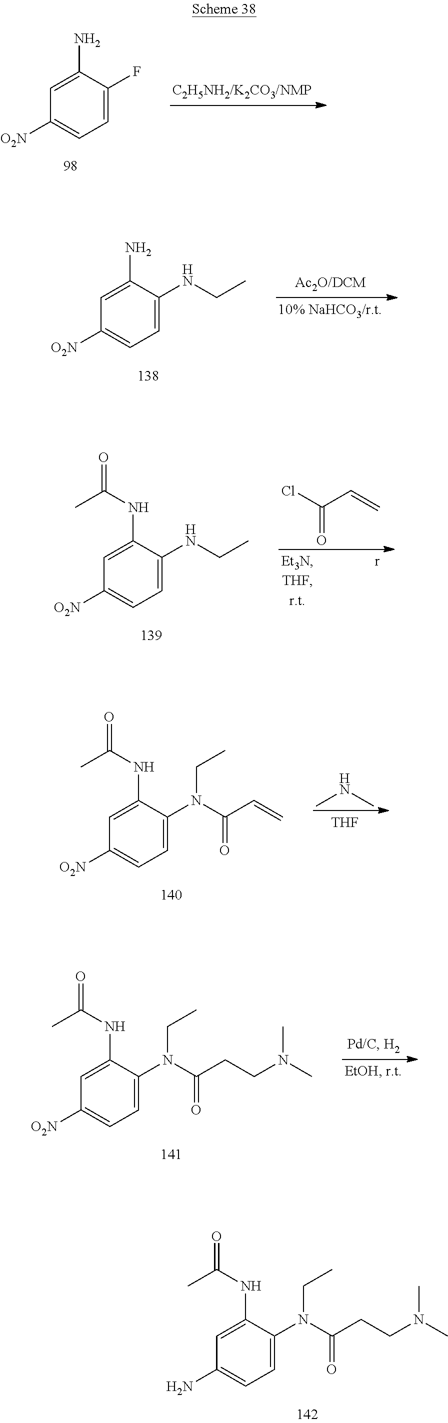

### Intermediate 142: N-1-ethyl-4-nitrobenzene-1,2-diamine (138)

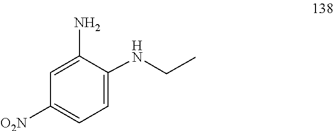

A mixture of 98 (10.0 g, 64.1 mmol), K₂CO₃ (26.5 g, 256.6 mmol) and ethyl amine (9.0 g, 43.4 mmol) in NMP (50 mL) was stirred at 120° C. under Ar overnight. After cooling to room temperature, the mixture was poured into 100 mL of water. The precipitate was filtered and dried under vacuum to give the title product 138 as a red solid (7.1 g, yield 49%). MS-ESI (M+H)⁺: 181.2.

### N-(2-(ethylamino)-5-nitrophenyl)acetamide (139)

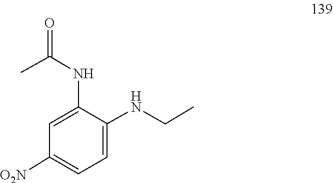 [SMILES: CCNc1ccc([N+](=O)[O-])cc1NC(C)=O]

A solution of 138 (2 g, 1.1 mmol), Ac₂O (1.0 g, 11.0 mmol) and NaHCO₃ (10 mg, 0.12 mmol) in water (10 mL) and dichloromethane (200 mL) was stirred at room temperature for 3 h. The reaction mixture was diluted with water and extracted with dichloromethane. The combined organics were dried over sodium sulfate and concentrated to give a crude product 139 as a brown solid (2.3 g, yield 89.1%), which was used to next step without further purification. MS-ESI (M+H)⁺: 223.1.

### N-(2-acetamido-4-nitrophenyl)-N-ethylacrylamide (140)

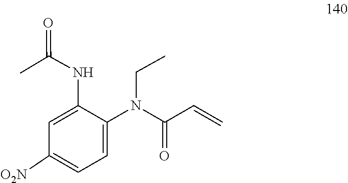 [SMILES: C=CC(=O)N(CC)c1ccc([N+](=O)[O-])cc1NC(C)=O]

A solution of 139 (2.3 g, 10.3 mmol), acryloyl chloride (1.86 g, 20.6 mmol) and Et₃N (2.8 mL, 20.6 mmol) in tetrahydrofuran (60 mL) was stirred at room temperature for 3 h. After diluting with water, the organic phase was extracted with ethyl acetate. The combined organics were dried over sodium sulfate and concentrated to give a crude product as a brown solid 140 (1.7 g, yield 60.2%), which was used to next step without further purification. MS-ESI (M+H)⁺: 292.2.

### N-(2-acetamido-4-nitrophenyl)-3-(dimethylamino)-N-ethylpropanamide (141)

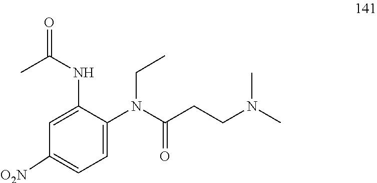 [SMILES: CCN(C(=O)CCN(C)C)c1ccc([N+](=O)[O-])cc1NC(C)=O]

A solution of 140 (1.7 g, 6.2 mmol) and dimethylamine (12.4 mL, 24.8 mmol, 2M in tetrahydrofuran) in tetrahydrofuran (20 mL) was stirred at room temperature for 30 min. The reaction mixture was concentrated to give a crude product as a brown solid 141 (1.8 g, yield 90.2%), which was used to next step without further purification. MS-ESI (M+H)⁺: 322.2.

### N-(2-acetamido-4-aminophenyl)-3-(dimethylamino)-N-ethylpropanamide (142)

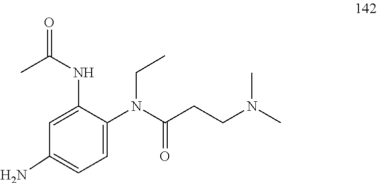 [SMILES: CCN(C(=O)CCN(C)C)c1ccc(N)cc1NC(C)=O]

A mixture of Pd/C (600 mg) and 141 (1.8 g, 5.6 mmol) in methanol (50 mL) was stirred at room temperature under hydrogen (balloon) for 23 h. The catalyst was filtered out and the filtrate was concentrated under vacuum to afford 142 (1.2 g, yield 73.6%). (MS-ESI) (M+H)⁺: 292.2. ¹H NMR (400 MHz, DMSO-d6) δ 8.99 (s, 1H), 7.05 (s, 1H), 6.80 (d, J=12.0 Hz, 1H), 6.37-6.34 (m, 1H), 5.30 (s, 2H), 3.89-3.85 (m, 1H), 3.39-3.33 (m, 2H), 3.02-2.95 (m, 1H), 2.64-2.52 (m, 2H), 2.15 (s, 6H), 2.02 (s, 3H), 0.93 (t, J=8.4 Hz, 3H).

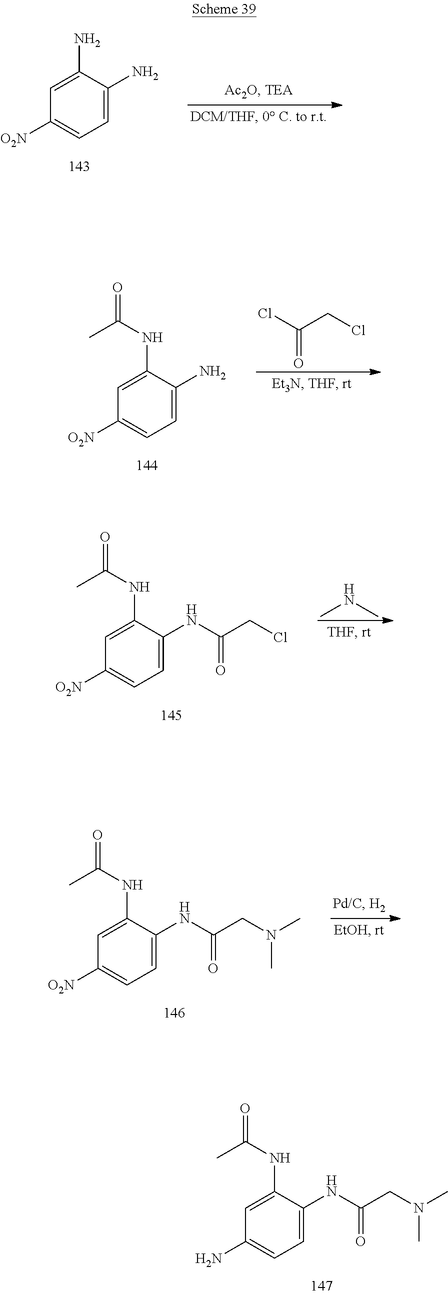

### Intermediate 147: N-(2-amino-5-nitrophenyl)acetamide (144)

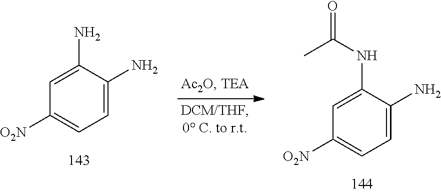

The mixture of 143 (1 g, 6.53 mmol) in dichloromethane (20 mL) and tetrahydrofuran (10 mL) were added Et₃N (1.82 mL, 13.06 mmol) and Ac₂O (0.68 mL, 7.18 mmol) at 0° C. under Ar. The mixture was stirred at 25° C. under Ar for 16 h. The reaction was quenched with H₂O (40 mL) and extracted with ethyl acetate (100 mL*5). The organic phase was dried by Na₂SO₄, filtered and concentrated in vacuo to give 144 (1.3 g, 6.53 mmol, ˜100% yield) as yellow solid, which was used in the next step without further purification. MS-ESI (M+H⁺+MeCN): 237.1. ¹H NMR (400 MHz, DMSO) δ 9.19 (s, 1H), 8.22 (d, J=2.6 Hz, 1H), 7.83 (dd, J=9.0, 2.7 Hz, 1H), 6.74 (d, J=9.1 Hz, 1H), 6.49 (s, 2H), 2.07 (s, 3H).

### N-(2-acetamido-4-nitrophenyl)-2-chloroacetamide (145)

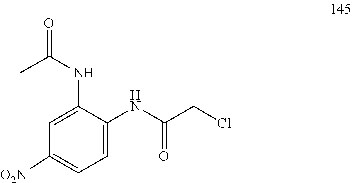 [SMILES: CC(=O)Nc1cc([N+](=O)[O-])ccc1NC(=O)CCl]

To the mixture of 144 (1.1 g, 5.64 mmol) in tetrahydrofuran (30 mL) were added Et₃N (1.57 mL, 11.27 mmol) and 2-chloroacetyl chloride (0.90 mL, 11.27 mmol) under Ar. The mixture was stirred at 25° C. under Ar for 16 h. The mixture was quenched with H₂O (50 mL), extracted with ethyl acetate (50 mL*5). The organic phase was dried by Na₂SO₄, filtered and concentrated in vacuo. The residue was purified by flash column chromatograph (petroleum ether/ethyl acetate=5/1-2/1˜1/1-1:2) to give 145 (1.2 g, 78% yield) as a white solid. MS-ESI (M−H⁺): 270.2. ¹H NMR (400 MHz, CDCl₃) δ 9.23 (s, 1H), 8.20 (d, J=2.5 Hz, 1H), 8.16 (dd, J=8.9, 2.5 Hz, 1H), 8.02 (d, J=8.8 Hz, 1H), 4.23 (s, 2H), 2.29 (s, 3H).

### N-(2-acetamido-4-nitrophenyl)-2-(dimethylamino)acetamide (146)

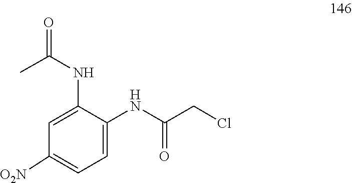 [SMILES: CC(=O)Nc1cc([N+](=O)[O-])ccc1NC(=O)CCl]

To the mixture of 145 (1.2 g, 4.42 mmol) in tetrahydrofuran (22 mL) was added dimethyl amine (4.42 mL, 8.84 mmol, 2M in tetrahydrofuran) under Ar. The mixture was stirred at 25° C. under Ar for 16 h. The mixture was concentrated in vacuo. The residue was purified by flash column chromatograph (dichloromethane/methanol=20/1) to give 146 (1.1 g, 88.69% yield) as a yellow solid. MS-ESI (M+H⁺): 281.2.

### N-(2-acetamido-4-aminophenyl)-2-(dimethylamino)acetamide (147)

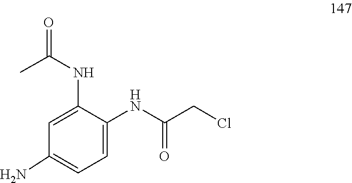 [SMILES: CC(=O)Nc1cc(N)ccc1NC(=O)CCl]

To the mixture of 146 (1.1 g, 3.92 mmol) in ethanol (20 mL) was added Pd/C (418 mg, 0.39 mmol). The mixture was stirred at 25° C. under H₂ (15 psi) for 16 h. The mixture was filtered and concentrated in vacuo to give 147 (1 g, ˜100% yield) as a grey solid, which was used in the next step without further purification. MS-ESI (M+H⁺): 251.2.

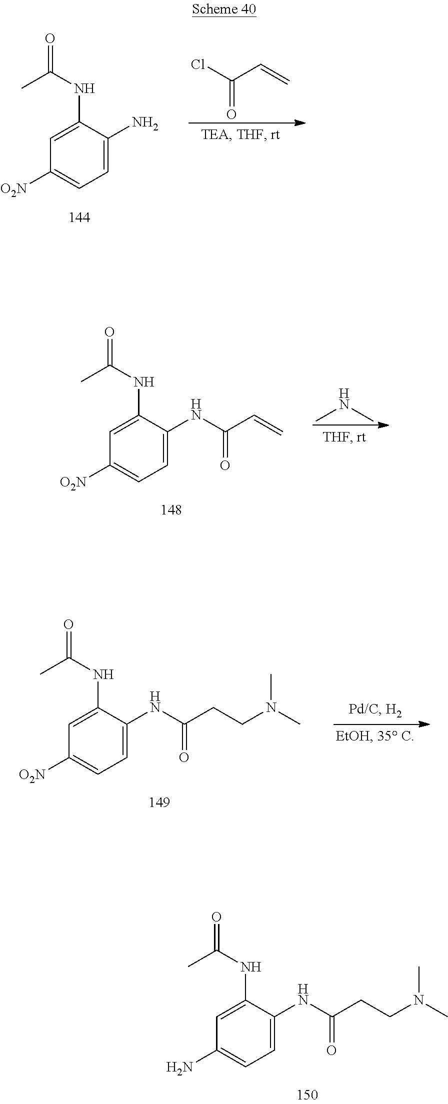

### N-(2-acetamido-4-nitrophenyl)acrylamide (148)

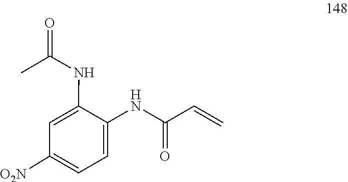 [SMILES: C=CC(=O)Nc1ccc([N+](=O)[O-])cc1NC(C)=O]

To the mixture of 144 (2 g, 10.25 mmol) in tetrahydrofuran (40 mL) were added Et₃N (2.14 mL, 15.37 mmol) and acryloyl chloride (1 mL, 12.30 mmol) under Ar. The mixture was stirred at 25° C. under Ar for 16 h. The reaction was concentrated in vacuo to give 148 (3 g, ˜100% yield) as yellow solid, which was used in the next step without further purification. MS-ESI (M+H⁺): 250.1.

### N-(2-acetamido-4-nitrophenyl)-3-(dimethylamino)propanamide (149)

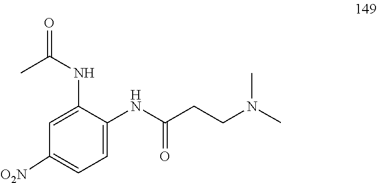 [SMILES: CC(=O)Nc1cc([N+](=O)[O-])ccc1NC(=O)CCN(C)C]

To the mixture of 148 (3 g, 10.25 mmol) in tetrahydrofuran (40 mL) was added dimethylamine (12.81 mL, 25.63 mmol, 2M in tetrahydrofuran). The mixture was stirred at 25° C. under Ar for 6 h. The mixture was concentrated in vacuo and the residue was purified by flash column chromatograph (dichloromethane/methanol=20/1-10/1) to give 149 (1.89 g, 62.65% yield) as a yellow solid. MS-ESI (M+H⁺): 295.2.

### N-(2-acetamido-4-aminophenyl)-3-(dimethylamino)propanamide (150)

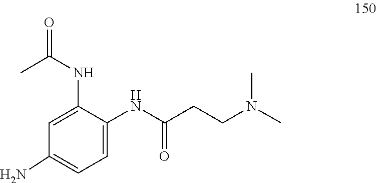 [SMILES: CC(=O)Nc1cc(N)ccc1NC(=O)CCN(C)C]

To the mixture of 149 (1 g, 3.40 mmol) in ethanol (30 mL) was added Pd/C (362 mg, 0.34 mmol, 10%). The mixture was stirred at 35° C. under H₂ (15 psi) for 16 h. The mixture was filtered and concentrated to give 150 (1 g, 3.4 mmol, ˜100% yield) as a white solid, which was used in the next step without further purification. MS-ESI (M+H⁺): 265.1.

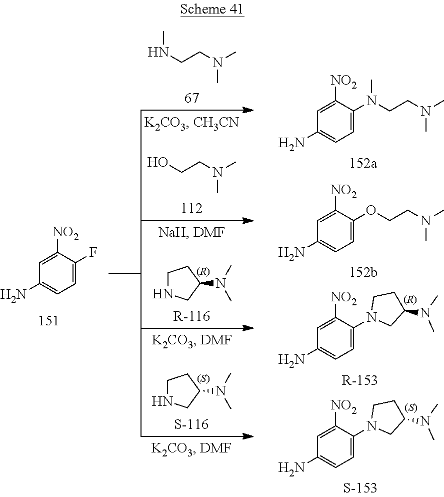

### Intermediate 152a: N¹-(2-(dimethylamino)ethyl)-N¹-methyl-2-nitrobenzene-1,4-diamine(152a)

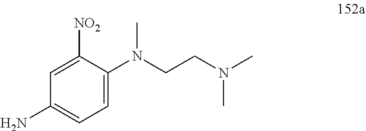 [SMILES: CN(C)CCN(C)c1ccc(N)cc1[N+](=O)[O-]]

A mixture of 151 (5.0 g, 32.02 mmol), N¹,N¹,N²-trimethylethane-1,2-diamine 67 (4.91 g, 48.05 mmol) and K2C03 (8.93 g, 64.61 mmol) in acetonitrile (80 ml) was stirred at refluxing temperature overnight. The reaction mixture was filtered and concentrated. The crude product was dissolved in ethyl acetate (200 ml) and washed by H₂O (50 ml*3). The organic layer was concentrated and the residue was purified by flash column chromatography (dichloromethane/methanol=20/1) to give the compound 152a (4.80 g, yield 62.98%).

### Intermediate 152b: 4-(2-(dimethylamino)ethoxy)-3-nitroaniline (152b)

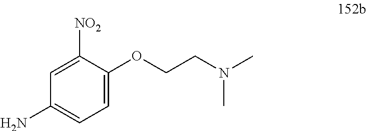 [SMILES: CN(C)CCOc1ccc(N)cc1[N+](=O)[O-]]

A mixture of 151 (497 mg, 3.18 mmol) and 112 (482 mg, 5.41 mmol) in DMF (20 mL) was added NaH (300 mg, 12 mmol). It was stirred at room temperature for 6 h followed by quenching with H₂O. The mixture was purified by flash column chromatography (dichloromethane/methanol=20/1) to give 152b as an oil (0.6 g, ˜84% yield).

### Intermediate R-153: (R)-1-(4-amino-2-nitrophenyl)-N,N-dimethylpyrrolidin-3-amine (R-153)

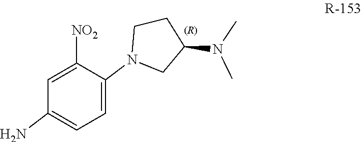

A mixture of 151 (1.62 g, 10.37 mmol), (R)—N,N-dimethylpyrrolidin-3-amine R-116 (1.23 g, 10.77 mmol) and K2CO₃ (2.81 g, 20.33 mmol) in DMF (30 mL) was stirred at 100° C. for 6 h. The reaction mixture was quenched by H₂O (200 mL) and diluted with ethyl acetate (200 mL). The organic layer was washed by H₂O (50 ml*3) and concentrated. The residue was purified by flash column chromatography (dichloromethane/methanol=20/1) to give the compound R-153 (2.6 g, yield ˜100%).

### Intermediate S-153: (S)-1-(4-amino-2-nitrophenyl)-N,N-dimethylpyrrolidin-3-amine (S-153)

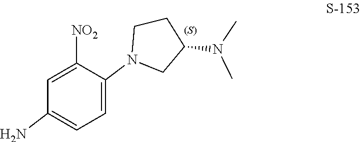

A mixture of 151 (1.23 g, 7.87 mmol), (S)—N,N-dimethylpyrrolidin-3-amine S-116 (1.0 g, 8.75 mmol) and K2CO₃ (2.19 g, 15.84 mmol) in DMF (6 mL) was stirred at 100° C. overnight. The reaction mixture was quenched by H₂O (50 mL) and diluted with ethyl acetate (50 mL). The organic layer was washed by H₂O (50 ml*3) and concentrated. The residue was purified by flash column chromatography (dichloromethane/methanol=20/1) to give the compound S-153 (1.9 g, yield 96.56%).

### Synthesis of Compounds of Formula III

### Procedure for the Synthesis of 156

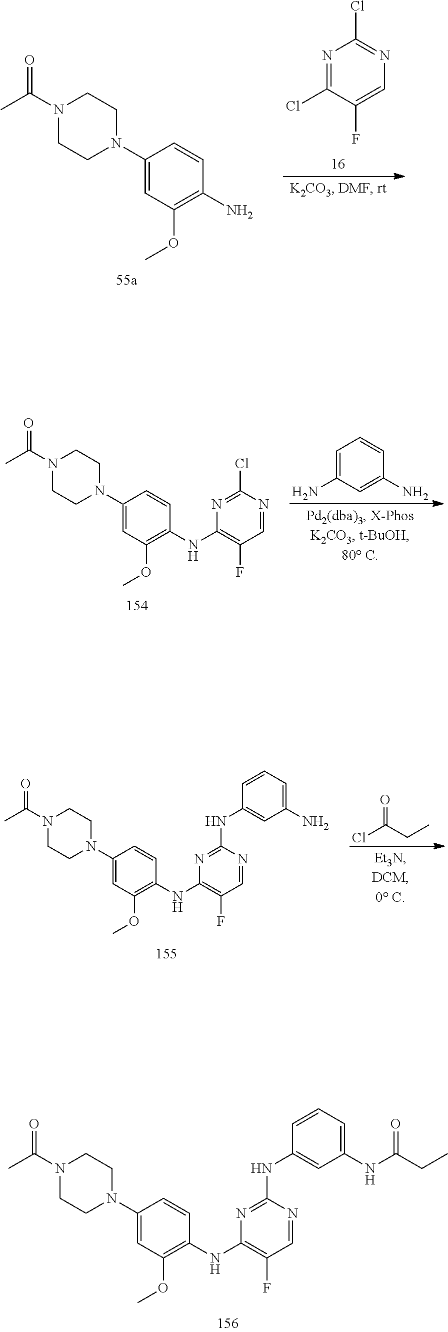

### 1-(4-(4-((2-chloro-5-fluoropyrimidin-4-yl)amino)-3-meth oxyphenyl)piperazin-1-yl)ethanone (154)

To the mixture of 55a (3.02 g, 12.11 mmol) and 2,4-dichloro-5-fluoropyrimidine 16 (2.39 g, 14.31 mmol) in DMF (35 mL) was added K₂CO₃ (3.11 g, 22.54 mmol). The mixture was stirred at room temperature overnight. The mixture was diluted with water (40 mL), then extracted with ethyl acetate (30 mL*3). The organic phase was combined and washed with H₂O (40 mL*2), dried with Na₂SO₄. The organic phase was concentrated to give the product 154 (4.0 g, yield 96.9%) as almost white solid.

### 1-(4-(4-((2-((3-aminophenyl)amino)-5-fluoropyrimidin-4-yl) amino)-3-methoxyphenyl)piperazin-1-yl)ethanone (155)

A mixture of 154 (2.98 g, 7.85 mmol), benzene-1,3-diamine (1.78 g, 16.46 mmol), Pd₂(dba)₃ (1.71 g, w %=57%), X-Phos (1.69 g, w %=57%) and K₂CO₃ (3.42 g 24.78 mmol) in t-BuOH (15 mL) was stirred at 80° C. for 4 h. After cooling to 45° C., the mixture was filtered off and the filtrate was concentrated under reduced pressure. The residue diluted with ethyl acetate (100 mL). The organic phase was washed with H₂O (50 mL*2), dried with Na₂SO₄. After concentration, the residue was purified by flash column chromatography (dichloromethane/methanol=20/1) to give the product 155 (1.5 g, yield 42.3%) as a gray solid.

### N-(3-((4-((4-(4-acetylpiperazin-1-yl)-2-methoxyphenyl) amino)-5-fluoropyrimidin-2-yl)amino)phenyl)propionamide (156)

A mixture of 155 (0.48 g, 1.06 mmol), Et₃N (0.24 g, 2.37 mmol) in dichloromethane (10 mL) was stirred at 0° C. for 15 min. Then propionyl chloride (0.18 g, 1.95 mmol) was added dropwise and the mixture was stirred at 0° C. for another 1 h. The reaction mixture was quenched by saturated NHCO₃ (10 mL) and diluted with dichloromethane (10 mL). The organic phase was washed with H₂O (10 mL*2), dried with Na₂SO₄. After concentration, the residue was purified by flash column chromatography (dichloromethane/methanol=15/1) to give the desired product 156 (0.13 g, yield 24.1%) as a white solid. MS-ESI (M+H)⁺: 508.5. ¹H NMR (500 MHz, DMSO-d₆) δ 9.71 (s, 1H), 9.07 (s, 1H), 8.24 (d, J=2.0 Hz, 1H), 8.00 (d, J=3.6 Hz, 1H), 7.67 (s, 1H), 7.60 (d, J=8.7 Hz, 1H), 7.36 (dd, J=8.2, 1.2 Hz, 1H), 7.16 (d, J=8.1 Hz, 1H), 7.02 (t, J=8.1 Hz, 1H), 6.71 (d, J=2.6 Hz, 1H), 6.51 (dd, J=8.7, 2.6 Hz, 1H), 3.81 (s, 3H), 3.61 (q, J=6.6 Hz, 4H), 3.18 (t, J=5.2 Hz, 2H), 3.12 (t, J=5.3 Hz, 2H), 2.30 (q, J=7.5 Hz, 2H), 2.07 (s, 3H), 1.07 (t, J=7.6 Hz, 3H).

### Procedure for the Synthesis of 158

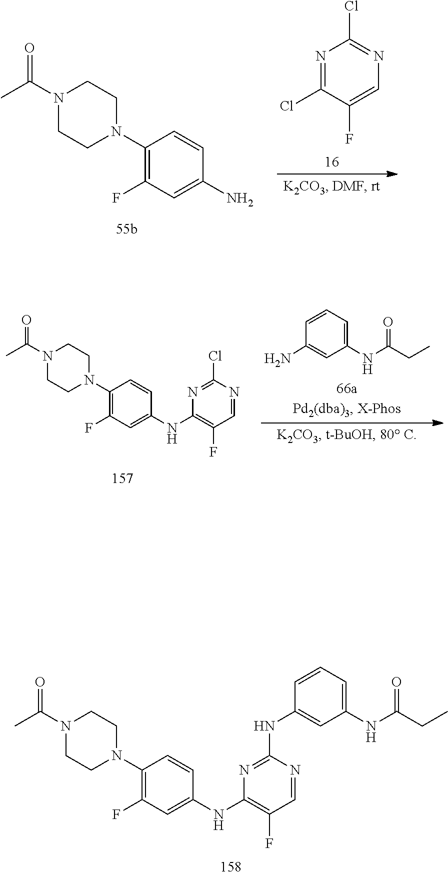 [SMILES: CC(=O)N1CCN(c2ccc(N)cc2F)CC1 ; Fc1cnc(Cl)nc1Cl ; CC(=O)N1CCN(c2ccc(Nc3nc(Cl)ncc3F)cc2F)CC1 ; CCC(=O)Nc1cccc(N)c1 ; CCC(=O)Nc1cccc(Nc2ncc(F)c(Nc3ccc(N4CCN(C(C)=O)CC4)c(F)c3)n2)c1]

### 1-(4-(4-((2-chloro-5-fluoropyrimidin-4-yl)amino)-2-fluoro phenyl)piperazin-1-yl)ethanone (157)

Synthesis of 157 was similar to that of 154. Using 55b (4.02 g, 16.94 mmol) and 2,4-dichloro-5-fluoropyrimidine 16 (3.47 g, 20.78 mmol) to give 157 as a gray solid (1.83 g, yield 29.4%).

### N-(3-((4-((4-(4-acetylpiperazin-1-yl)-3-fluorophenyl) amino)-5-fluoropyrimidin-2-yl)amino)phenyl)propionamide (158)

A mixture of 157 (0.62 g, 1.69 mmol), 66a (0.53 g, 3.23 mmol), Pd₂(dba)₃ (0.57 g, w %=92%), X-Phos (0.55 g, w %=89%) and K₂CO₃ (0.78 g 5.65 mmol) in t-BuOH (30 mL) was stirred at 80° C. for 2.5 h. After cooling to 45° C., the mixture was filtered off and the filtrate was concentrated under reduced pressure. The residue diluted with ethyl acetate (50 mL). The organic phase was washed with H₂O (20 mL*2), dried with Na₂SO₄. After concentration, the residue was purified by flash column chromatography (dichloromethane/methanol=20/1) to give the desired product 158 (0.28 g, yield 33.5%) as an off-white solid. MS-ESI (M+H)⁺: 496.2.

### Procedure for the Synthesis of 160

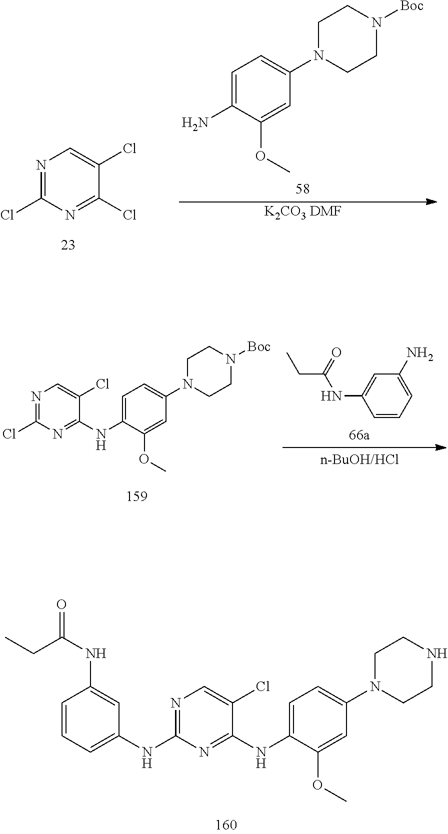

### tert-butyl 4-(4-((2,5-dichloropyrimidin-4-yl)amino)-3-methoxyphenyl) piperazine-1-carboxylate (159)

58 (2.9 g, 9.4 mmol) and 23 (1.8 g, 9.9 mmol), K₂CO₃ (2.4 g, 19.0 mmol) were treated with DMF (20 mL) and allowed to stir at 80° C. for 16 h. The N,N-dimethylformamide of the mixture was removed under reduced pressure and added with water to form a solid. The solid was filtered to obtain the target compound 159 (3.0 g, 69% yield) as a white solid, which was used to next step directly without further purification. (MS-ESI) (M+H)⁺: 454.0.

### N-(3-((5-chloro-4-((2-methoxy-4-(piperazin-1-yl)phenyl)amino)pyrimidin-2-yl)amino)phenyl)propionamide (160)

159 (500 mg, 1.1 mmol) and 66a (195 mg, 1.2 mmol) were treated with butyl alcohol (20 mL) and catalytic amount of HCl. The mixture was allowed to stir at 115° C. for 16 h. After cooling to room temperature, the mixture was diluted with ethyl acetate (200 mL). The organic phase was washed with H₂O (100 mL) and brine (100 mL*2), dried with Na₂SO₄. After concentration, the residue was purified by Pre-HPLC (MeCN/0.05% formic acid in water from 0 to 100%)) to give the title product as FA salt, which was neutralized with saturated NaCO₃ to give 160 as a white solid (109.9 mg, yield 19.1%). MS-ESI (M+H)⁺: 482.1. ¹H NMR (400 MHz, DMSO-d6) δ 9.77 (s, 1H), 9.25 (s, 1H), 8.06 (s, 1H), 7.97 (s, 1H), 7.77 (d, J=8.6 Hz, 1H), 7.68 (s, 1H), 7.33 (d, J=7.9 Hz, 1H), 7.17 (d, J=7.9 Hz, 1H), 7.04 (t, J=8.1 Hz, 1H), 6.67 (s, 1H), 6.45 (d, J=7.6 Hz, 1H), 3.81 (s, 3H), 3.40 (s, 2H), 3.04 (s, 4H), 2.85 (s, 2H), 2.30 (q, J=7.5 Hz, 2H), 1.06 (t, J=7.5 Hz, 3H).

### Procedure for the Synthesis of 161

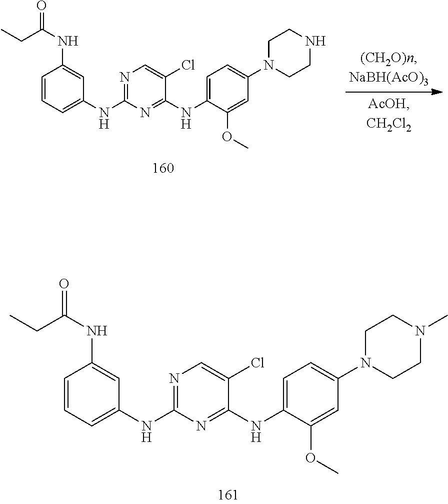 [SMILES: CCC(=O)Nc1cccc(Nc2ncc(Cl)c(Nc3ccc(N4CCNCC4)cc3OC)n2)c1 ; CCC(=O)Nc1cccc(Nc2ncc(Cl)c(Nc3ccc(N4CCN(C)CC4)cc3OC)n2)c1]

### N-(3-((5-chloro-4-((2-methoxy-4-(4-methylpiperazin-1-yl)phenyl)amino)pyrimidin-2-yl)amino)phenyl)propionamide (161)

A mixture of 160 (500 mg, 1.04 mmol) and (CH₂O)n (312 mg, 10.4 mmol) in dichloromethane (50 mL) was treated with catalytic amount of acetic acid and the mixture was allowed to stir at room temperature for 1 h. Then NaBH(OAc)₃ (254 mg 1.2 mmol) was added and the resulting mixture was stirred at room temperature for another 16 h. After concentration, the residue was purified by Pre-HPLC (MeCN/0.05% NH₄OH in water from 0 to 100%) to afford 161 (130 mg, yield 22%) as a yellow solid. MS-ESI (M+H)⁺: 496.2. 1H NMR 1H NMR (400 MHz, DMSO-d6) δ 9.72 (s, 1H), 9.26 (s, 1H), 8.07 (s, 1H), 7.99 (s, 1H), 7.81 (d, J=8.6 Hz, 1H), 7.66 (s, 1H), 7.35 (d, J=8.0 Hz, 1H), 7.17 (d, J=7.8 Hz, 1H), 7.04 (t, J=8.0 Hz, 1H), 6.72 (d, J=1.8 Hz, 1H), 6.49 (d, J=8.7 Hz, 1H), 3.83 (s, 3H), 3.60 (d, J=4.2 Hz, 4H), 3.17 (s, 2H), 3.11 (s, 2H), 2.29 (q, J=7.5 Hz, 2H), 2.06 (s, 3H), 1.06 (t, J=7.5 Hz, 3H).

### Procedure for the Synthesis of 162

### N-(3-((4-((4-(4-acetylpiperazin-1-yl)-2-methoxyphenyl)amino)-5-chloropyrimidin-2-yl)amino)phenyl)propionamide (162)

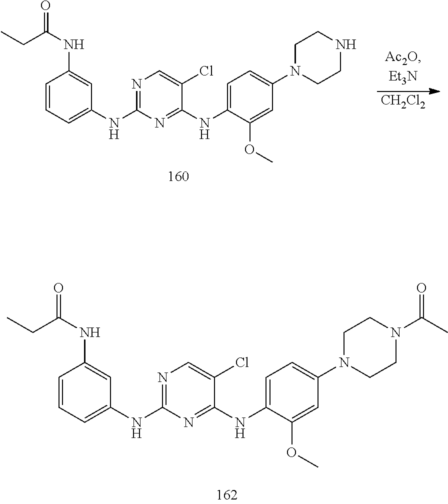

A mixture of 161 (800 mg, 1.6 mmol), Ac₂O (245 mg, 2.4 mmol) and Et₃N (323 g, 3.2 mmol) in dichloromethane (100 mL) was stirred at room temperature for 2 h. After concentration, the residue was purified by Pre-HPLC (MeCN/0.05% NH4OH in water from 0 to 100%) to afford 162 (160 mg, yield 22%) as a yellow solid. MS-ESI (M+H)⁺: 512.2. ¹H NMR (400 MHz, DMSO) δ 9.72 (s, 1H), 9.26 (s, 1H), 8.07 (s, 1H), 7.99 (s, 1H), 7.81 (d, J=8.6 Hz, 1H), 7.66 (s, 1H), 7.35 (d, J=8.1 Hz, 1H), 7.17 (d, J=7.9 Hz, 1H), 7.04 (t, J=8.1 Hz, 1H), 6.72 (d, J=2.0 Hz, 1H), 6.48 (dd, J=8.7, 2.0 Hz, 1H), 3.83 (s, 3H), 3.60 (d, J=4.3 Hz, 4H), 3.17 (s, 2H), 3.11 (s, 2H), 2.29 (q, J=7.5 Hz, 2H), 2.06 (s, 3H), 1.06 (t, J=7.5 Hz, 3H).

### Procedure for the Synthesis of 164

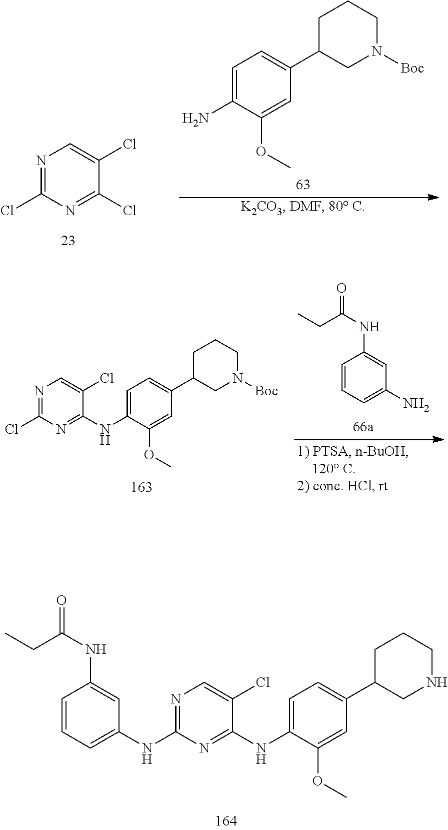 [SMILES: Clc1ncc(Cl)c(Cl)n1 ; COc1cc(C2CCCN(C(=O)OC(C)(C)C)C2)ccc1N ; "K2CO3, DMF, 80° C." ; COc1cc(C2CCCN(C(=O)OC(C)(C)C)C2)ccc1Nc1nc(Cl)ncc1Cl ; CCC(=O)Nc1cccc(N)c1 ; CCC(=O)Nc1cccc(Nc2ncc(Cl)c(Nc3ccc(C4CCCNC4)cc3OC)n2)c1]

### tert-butyl 3-(4-((2,5-dichloropyrimidin-4-yl)amino)-3-methoxyphenyl)piperidine-1-carboxylate (163)

To the mixture of 63 (535 mg, 1.75 mmol) and 2,4,5-trichloropyrimidine 23 (384 mg, 2.10 mmol) in DMF (10 mL) was added K₂CO₃ (483 mg, 3.49 mmol). The mixture was stirred at 80° C. under Ar for 16 h. The mixture was quenched with H₂O (15 mL), extracted with ethyl acetate (30 mL*3). The organic phase was washed with brine (20 mL*3), dried over Na₂SO₄, filtered and concentrated. The residue was purified by column chromatography (petroleum ether/ethyl acetate=10/1-5/1-3/1) to give 163 (630 mg, 79.59% yield) as yellow oil. MS-ESI (M+H)⁺: 453.2.

### N-(3-((5-chloro-4-((2-methoxy-4-(piperidin-3-yl)phenyl)amino)pyrimidin-2-yl)amino)phenyl)propionamide (164)

To the mixture of 163 (150 mg, 0.33 mmol) and N-(3-aminophenyl)propionamide 66a (71 mg, 0.43 mmol) in n-BuOH (3 mL) was added PTSA·H₂O (6 mg, 0.033 mmol). The mixture was stirred at 120° C. under Ar for 16 h. Conc.HCl (0.5 mL) was added to the mixture. The mixture was stirred at 25° C. under Ar for 3 h. The mixture was concentrated and purified by Prep-HPLC (FA) and basified to give 164 (44.09 mg, 27.70% yield) as a white solid. MS-ESI (M+H)⁺: 481.1. ¹H NMR (400 MHz, DMSO-d6): δ 9.76 (s, 1H), 9.34 (s, 1H), 8.13 (s, 1H), 8.04 (d, J=7.2 Hz, 2H), 7.71 (s, 1H), 7.32 (d, J=8.1 Hz, 1H), 7.20 (d, J=7.6 Hz, 1H), 7.06 (t, J=8.1 Hz, 1H), 6.95 (d, J=1.6 Hz, 1H), 6.77 (d, J=8.2 Hz, 1H), 3.86 (s, 3H), 3.04-2.90 (m, 2H), 2.59-2.54 (m, 2H), 2.48-2.44 (m, 1H), 2.30 (q, J=7.6 Hz, 2H), 1.89 (d, J=11.7 Hz, 1H), 1.67 (d, J=12.0 Hz, 1H), 1.62-1.54 (m, 1H), 1.49 (t, J=11.8 Hz, 1H), 1.07 (t, J=7.5 Hz, 3H).

### Procedure for the Synthesis of 165

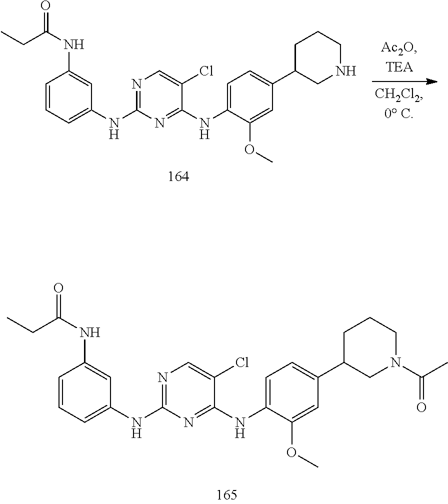 [SMILES: CCC(=O)Nc1cccc(Nc2ncc(Cl)c(Nc3ccc(C4CCCNC4)cc3OC)n2)c1 ; CCC(=O)Nc1cccc(Nc2ncc(Cl)c(Nc3ccc(C4CCCN(C(C)=O)C4)cc3OC)n2)c1]

### N-(3-((4-((4-(1-acetylpiperidin-3-yl)-2-methoxyphenyl)amino)-5-chloropyrimidin-2-yl)amino)phenyl)propionamide (165)

To the mixture of 164 (222 mg, 0.46 mmol) in dichloromethane (5 mL) were added Et₃N (0.08 mL, 0.55 mmol) and Ac₂O (0.05 mg, 0.55 mmol) at 0° C. under Ar. The mixture was stirred at 0° C. under Ar for 2 h. The mixture was quenched with H₂O (0.5 mL) and concentrated. The residue was purified by Prep-TLC (dichloromethane/methanol=20/1) to give 165 (118.02 mg, 48.89% yield) as white solid. MS-ESI (M+H)⁺: 523.2. ¹H NMR (400 MHz, DMSO-d6) δ 9.77 (s, 1H), 9.36 (d, J=4.8 Hz, 1H), 8.14 (d, J=2.1 Hz, 1H), 8.11-8.01 (m, 2H), 7.71 (s, 1H), 7.32 (s, 1H), 7.19 (d, J=8.0 Hz, 1H), 7.12-6.98 (m, 2H), 6.84 (dd, J=18.9, 8.2 Hz, 1H), 4.44 (t, J=11.6 Hz, 1H), 3.87 (s, 3H), 3.84-3.73 (m, 1H), 3.19-3.03 (m, 1H), 2.68 (t, J=11.9 Hz, 1H), 2.56 (t, J=13.1 Hz, 1H), 2.30 (q, J=7.5 Hz, 2H), 2.04 (s, 3H), 1.93 (d, J=11.0 Hz, 1H), 1.78-1.67 (m, 2H), 1.47-1.43 (m, 1H), 1.06 (dd, J=8.6, 6.4 Hz, 3H).

### Procedure for the Synthesis of 166

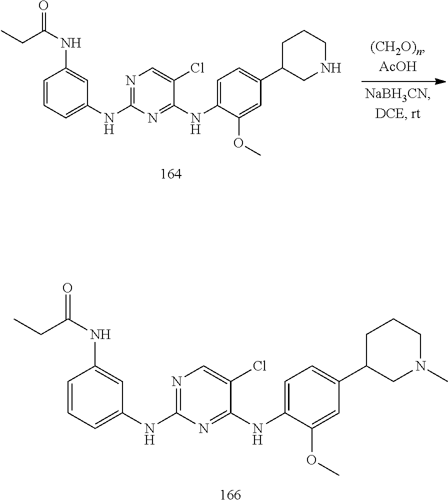 [SMILES: CCC(=O)Nc1cccc(Nc2ncc(Cl)c(Nc3ccc(C4CCCNC4)cc3OC)n2)c1 ; CCC(=O)Nc1cccc(Nc2ncc(Cl)c(Nc3ccc(C4CCCN(C)C4)cc3OC)n2)c1]

### N-(3-((5-chloro-4-((2-methoxy-4-(1-methylpiperidin-3-yl)phenyl)amino)pyrimidin-2-yl)amino)phenyl)propionamide (166)

To the mixture of 164 (222 mg, 0.46 mmol) in DCE (5 mL) were added (CH₂O)ₙ(28 mg, 0.92 mmol) and acetic acid (28 mg, 0.46 mmol). The mixture was stirred at 25° C. under Ar for 0.5 h. NaBH₃CN (44 mg, 0.69 mmol) was added to the mixture. The mixture was stirred at 25° C. under Ar for 16 h. The mixture was quenched with Et₃N (0.05 mL) and concentrated in vacuo. The residue was purified by Prep-TLC (dichloromethane/methanol=20/1) and further prep-HPLC (NH₃·H₂O) to give 166 (46.32 mg, 48.89% yield) as white solid. MS-ESI (M+H)⁺: 495.1. ¹H NMR (400 MHz, DMSO-d6) δ 9.75 (s, 1H), 9.34 (s, 1H), 8.13 (s, 1H), 8.09-7.99 (m, 2H), 7.71 (s, 1H), 7.31 (d, J=8.0 Hz, 1H), 7.20 (d, J=8.2 Hz, 1H), 7.07 (t, J=8.1 Hz, 1H), 6.98 (d, J=1.6 Hz, 1H), 6.79 (d, J=8.2 Hz, 1H), 3.86 (s, 3H), 2.83-2.75 (m, 3H), 2.30 (q, J=7.5 Hz, 2H), 2.19 (s, 3H), 1.92 (t, J=10.9 Hz, 2H), 1.86-1.77 (m, 1H), 1.75-1.70 (m, 1H), 1.63-1.59 (m, 1H), 1.45-1.40 (m, 1H), 1.07 (t, J=7.5 Hz, 3H).

### Procedure for the Synthesis of 169

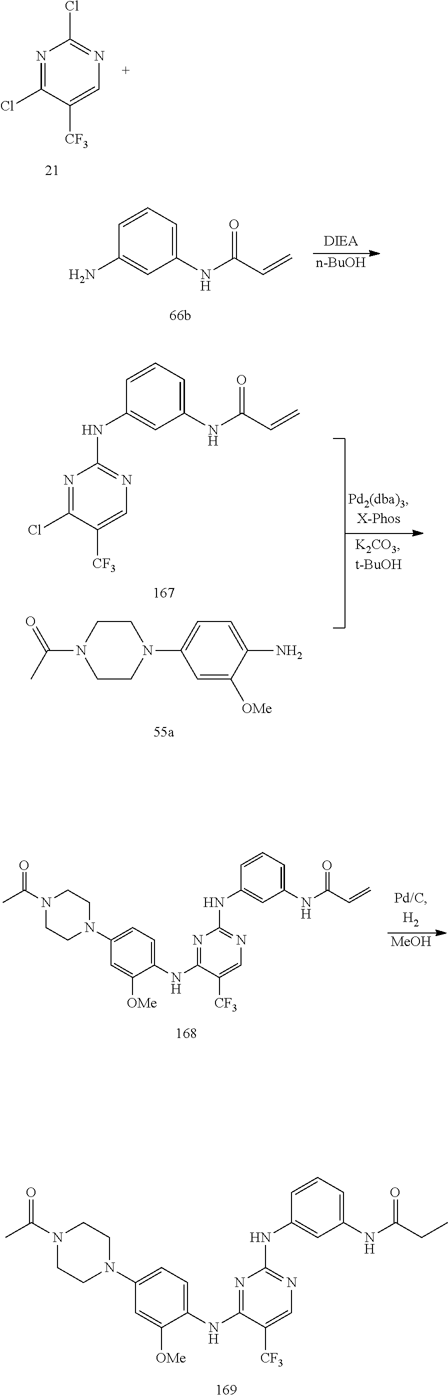

### N-(3-((4-chloro-5-(trifluoromethyl)pyrimidin-2-yl)amino)phenyl)acrylamide (167)

To the mixture of 66b (4.868 g, 30 mmol) in n-BuOH (50 mL) cooled to below 10° C., was slowly added 21 (6.658 g, 30 mmol) mixture in n-BuOH (4 mL) during about 30 min, after then, N,N-diisopropyl ethylamine (8.710 g, 60 mmol) was added. The mixture was warmed to room-temperature and stirred for another 4 h. The mixture was washed with water and extracted with dichloromethane. The organic phase was concentrated. The residue was purified by pre-HPLC to give 167 (1.530 g, 15% yield) as a white to brown solid. MS-ESI (M+H)⁺: 343.5.

### N-(3-((4-((4-(4-acetylpiperazin-1-yl)-2-methoxyphenyl)amino)-5-(trifluoromethyl)pyrimidin-2-yl)amino)phenyl)acrylamide (168)

To the mixture of 167 (1.123 g, 3.274 mmol) and 55a (0.785 g, 3.153 mmol) in t-BuOH (50 mL) were added K₂CO₃ (1.499 g, 10.86 mmol), Pd₂(dba)₃ (0.152 g, 0.166 mmol) and X-Phos (0.140 g, 0.295 mmol). The mixture was stirred at 85° C. overnight. After cooling to room temperature and concentration, the residue was purified by flash column chromatography (dichloromethane/methanol=30/1-20/1) to give 168 (0.172 g, 9.86% yield) as a brown solid. MS-ESI (M+H)⁺: 556.8.

### N-(3-((4-((4-(4-acetylpiperazin-1-yl)-2-methoxyphenyl)amino)-5-(trifluoromethyl)pyrimidin-2-yl)amino)phenyl)propionamide (169)

168 (60 mg, 0.108 mmol), Pd/C (5% w/w, 10 mg) and methanol (5 mL) was added in the flask. Then the mixture was replaced with H₂ for 3 times followed by stirring for 3 h at room temperature under H₂. The reaction mixture was filtered through Celite® and concentrated under reduced pressure to give a residue. The residue was purified by flash column chromatography (dichloromethane/methanol=30/1-20/1) to give 169 (30 mg, 50% yield) as a solid. MS-ESI (M+H)⁺: 558.5.

### Procedure for the Synthesis of 173

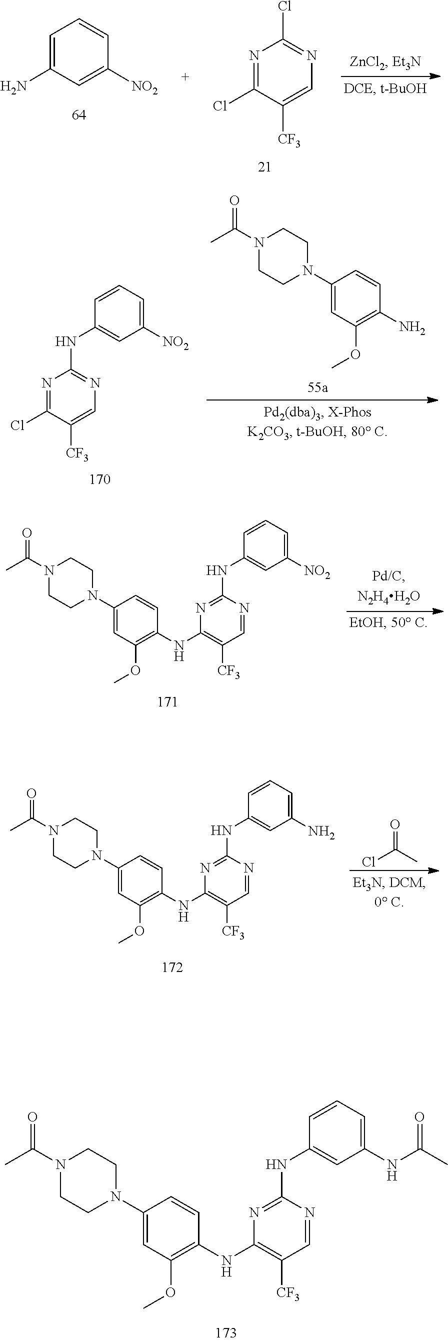

### 4-chloro-N-(3-nitrophenyl)-5-(trifluoromethyl)pyrimidin-2-amine (170)

A solution of 2,4-dichloro-5-(trifluoromethyl)pyrimidine 21 (1.08 g, 4.98 mmol) in DCE/t-BuOH (7.5 mL/7.5 mL) was stirred at 0° C. for 10 min. A solution of ZnCl₂ (1.0 M in THF, 20 ml, 20 mmol) was added dropwise and the mixture was stirred at 0° C. for another 1 h. Then 3-nitroaniline 64 was dissolved in DCE/t-BuOH (2.5 mL/2.5 mL) and slowly dripped into the reaction. After addition of Et₃N (1.0 g, 10 mmol), the mixture was transferred to room temperature overnight. The mixture was concentrated under reduced pressure. The residue dissolved with ethyl acetate (50 mL). The organic phase was washed with H₂O (20 mL*2), dried with Na₂SO₄. After concentration, the residue was purified by flash column chromatography (petroleum ether/ethyl acetate=10/1) to afford the product 170 (0.97 g, yield 61.8%) as a gray solid.

### 1-(4-(3-methoxy-4-((2-((3-nitrophenyl)amino)-5-(trifluoro methyl)pyrimidin-4-yl)amino)phenyl)piperazin-1-yl)ethanone (171)

A mixture of 170 (0.79 g, 2.48 mmol), 55a (0.91 g, 3.65 mmol), Pd₂(dba)₃ (0.30 g, w %=38%), X-Phos (0.32 g, w %=41%) and K₂CO₃ (0.73 g 6.09 mmol) in t-BuOH (25 mL) was stirred at 80° C. for 5 h. After cooling to 45° C., the mixture was filtered off and the filtrate was concentrated under reduced pressure. The residue diluted with ethyl acetate (50 mL). The organic phase was washed with H₂O (20 mL*2), dried with Na₂SO₄. After concentration, the residue was purified by flash column chromatography (dichloromethane/methanol=20/1) to give the desired product 171 (1.30 g, yield 98.7%) as a light yellow solid.

### 1-(4-(4-((2-((3-aminophenyl)amino)-5-(trifluoromethyl)pyri midin-4-yl)amino)-3-methoxyphenyl)piperazin-1-yl)ethanone (172)

To a solution of 171 (1.3 g, 2.45 mmol) in ethanol (10 mL), 5% Pd/C (0.36 g, w %=28%) and hydrazine hydrate (30 mL) were added. The mixture was stirred at 80° C. for 3.5 h. The mixture was concentrated under reduced pressure. The residue diluted with ethyl acetate (30 mL). The organic phase was washed with H₂O (10 mL*2), dried with Na₂SO₄. After concentration, the residue was purified by flash column chromatography (petroleum ether/ethyl acetate=8/1) to afford the product 172 (0.37 g, 30.2%) as an off-white solid.

### N-(3-((4-((4-(4-acetylpiperazin-1-yl)-2-methoxyphenyl)amino)-5-(trifluoromethyl)pyrimidin-2-yl)amino)phenyl)acetamide (173)

A mixture of 172 (0.20 g, 0.40 mmol), Et₃N (0.06 g, 0.59 mmol) in dichloromethane (5 mL) was stirred at 0° C. for 30 min. Then acetyl chloride (0.08 g, 1.02 mmol) was added dropwise and the mixture was stirred at 0° C. for another 1 h. The reaction mixture was quenched by saturated NHCO₃ (50 mL) and diluted with dichloromethane (20 mL). The organic phase was washed with H₂O (50 mL*2), dried with Na₂SO₄. After concentration, the residue was purified by flash column chromatography (dichloromethane/methanol=20/1) to afford the desired product 173 (0.11 g, 50.8%) as an off-white solid. MS-ESI (M+H)⁺: 544.7. ¹H NMR (500 MHz, DMSO-d₆) δ 9.88 (s, 1H), 9.68 (s, 1H), 8.31 (s, 1H), 7.96 (s, 1H), 7.59 (s, 2H), 7.35 (d, J=8.1 Hz, 1H), 7.19 (d, J=8.2 Hz, 1H), 7.01 (t, J=7.8 Hz, 1H), 6.73 (s, 1H), 6.49 (d, J=11.1 Hz, 1H), 3.79 (s, 3H), 3.61 (q, J=5.8 Hz, 4H), 3.20 (t, 2H), 3.13 (t, 2H), 2.07 (s, 3H), 2.02 (s, 3H).

### Procedure for the Synthesis of 177

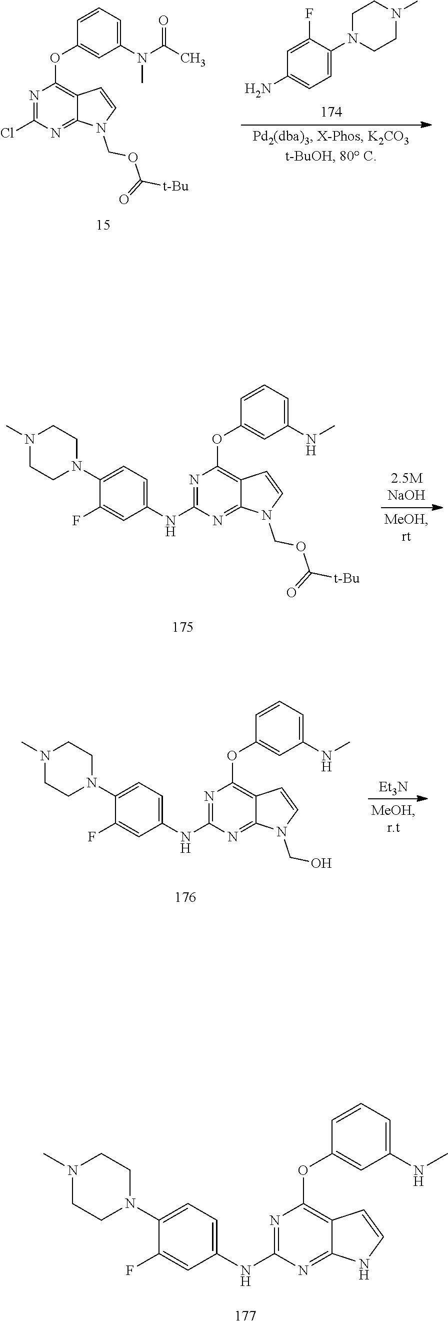

### (2-((3-fluoro-4-(4-methylpiperazin-1-yl)phenyl)amino)-4-(3-(methylamino)phenoxy)-7H-pyrrolo[2,3-d]pyrimidin-7-yl)methyl Pivalate (175)

A mixture of 15 (1.40 g, 2.89 mmol), 3-fluoro-4-(4-methylpiperazin-1-yl)aniline 174 (0.63 g, 3.01 mmol), Pd₂(dba)₃ (0.13 g, w %=9%), X-Phos (0.24 g, w %=17%) and K₂CO₃ (0.75 g 5.43 mmol) in t-BuOH (20 mL) was stirred at 100° C. for 4 h. After cooling to 45° C., the mixture was filtered off and the filtrate was concentrated under reduced pressure. The residue was purified by flash column chromatography (dichloromethane/methanol=50/1) to give the product 175 (1.48 g, yield 91.4%) as a gray solid.

### (2-((3-fluoro-4-(4-methylpiperazin-1-yl)phenyl)amino)-4-(3-(methylamino)phenoxy)-7H-pyrrolo[2,3-d]pyrimidin-7-yl)methanol (176)

A solution of 175 (1.45 g, 2.58 mmol) in methanol (32 mL) was stirred at 0° C. for 10 min. Then 2.5M NaOH (11 mL) was added dropwise and the mixture was stirred at room temperature for another 1.5 h. The reaction mixture was diluted with ethyl acetate (80 mL). The organic phase was washed with H₂O (30 mL*2), dried with Na₂SO₄. After concentration, the residue was purified by flash column chromatography (dichloromethane/methanol=100/1) to give the product 176 (1.10 g, yield 89.22%) as a white solid.

### N-(3-fluoro-4-(4-methylpiperazin-1-yl)phenyl)-4-(3-(methyl amino)phenoxy)-7H-pyrrolo[2,3-d]pyrimidin-2-amine (177)

A solution of 176 (1.10 g, 2.30 mmol) in methanol (6 mL) was stirred at room temperature for 10 min. Then the Et₃N (3.64 g, 36.00 mmol) was added dropwise and the mixture was stirred at room temperature for another 1.5 h. The reaction mixture was concentrated and dissolved in dichloromethane (50 mL). The organic phase was washed with H₂O (20 mL*2), dried with Na₂SO₄. After concentration, the residue was purified by flash column chromatography (dichloromethane/methanol=50/1) to give the desired product 177 (0.99 g, yield 96.4%) as a white solid. MS-ESI (M+H)⁺: 448.7. ¹H NMR (500 MHz, DMSO-d₆) δ 11.56 (s, 1H), 9.14 (s, 1H), 7.70 (d, 1H), 7.31 (dd, 1H), 7.16 (t, J=8.0 Hz, 1H), 7.05 (td, 1H), 6.83 (t, J=9.4 Hz, 1H), 6.47 (dd, 1H), 6.42 (dd, 1H), 6.39 (t, J=2.1 Hz, 1H), 6.15 (dd, J=3.3, 1.8 Hz, 1H), 5.86 (q, J=4.9 Hz, 1H), 2.91 (s, 4H), 2.67 (d, J=5.0 Hz, 3H), 2.45 (s, 4H), 2.21 (s, 3H).

### Procedure for the Synthesis of 180, 181a and 181b

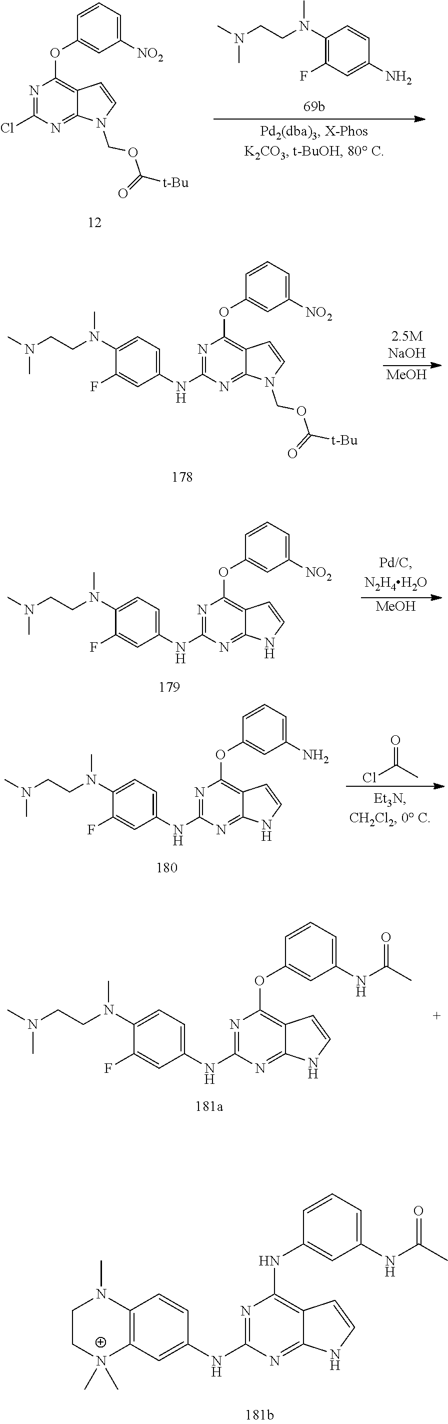

### (2-((4-((2-(dimethylamino)ethyl)(methyl)amino)-3-fluorophen yl)amino)-4-(3-nitrophenoxy)-7H-pyrrolo[2,3-d]pyrimidin-7-yl)methyl Pivalate (178)

A mixture of 69b (1.80 g, 8.52 mmol), 12 (3.79 g, 9.36 mmol), Pd₂(dba)₃ (0.77 g, w %=43%), X-Phos (0.77 g, w %=43%) and K₂CO₃ (3.48 g 25.22 mmol) in t-BuOH (20 mL) was stirred at 80° C. for 7 h. After cooling to 45° C., the mixture was filtered off and the filtrate was concentrated under reduced pressure. The residue was dissolved in ethyl acetate (50 mL), and washed with H₂O (20 mL*2), dried with Na₂SO₄. After concentration, the residue was purified by flash column chromatography (dichloromethane/methanol=15/1) to give the product 178 (2.0 g, yield 40.5%) as a gray solid.

### N¹-(2-(dimethylamino)ethyl)-2-fluoro-N1-methyl-N⁴-(4-(3-nitrophenoxy)-7H-pyrrolo[2,3-d]pyrimidin-2-yl)benzene-1,4-diamine (179)

A solution of 178 (2.00 g, 3.45 mmol) in methanol (10 mL) was stirred at 0° C. for 10 min. Then 2.5M NaOH (10 mL) was added dropwise and the mixture was stirred at room temperature for another 2 h. The reaction mixture was concentrated under reduced pressure. The residue was diluted with ethyl acetate (60 mL) and washed with H₂O (20 mL*2), dried with Na₂SO₄. The organic phase was concentrated to give the crude product 179 (2.0 g), which was used in the next step without future purification.

### N⁴-(4-(3-aminophenoxy)-7H-pyrrolo[2,3-d]pyrimidin-2-yl)-N¹-(2-(dimethylamino)ethyl)-2-fluoro-N1-methylbenzene-1,4-diamine (180)

To a solution of 179 (2.0 g, 4.30 mmol) in methanol (20 mL), 5% Pd/C (1.25 g, w %=10%) and hydrazine hydrate (50 mL) were added. The mixture was stirred at 85° C. for 2 h. The mixture was filtered off and the filtrate was concentrated under reduced pressure. The residue diluted with ethyl acetate (100 mL). The organic phase was washed with H₂O (30 mL*2), dried with Na₂SO₄. The organic phase was concentrated to give the desired product 180 (0.383 g, total yield 25.4% over two steps) as an off-white solid. MS-ESI (M+H)⁺: 436.4. ¹H NMR (500 MHz, DMSO-d₆) δ 11.60 (s, 1H), 9.18 (s, 1H), 7.72 (d, J=15.4 Hz, 1H), 7.32 (d, J=8.1 Hz, 1H), 7.08 (t, J=8.0 Hz, 1H), 7.04 (s, 1H), 6.94 (t, J=9.4 Hz, 1H), 6.49 (d, J=7.9 Hz, 1H), 6.41 (s, 1H), 6.37 (d, J=7.5 Hz, 1H), 6.11 (s, 1H), 5.29 (s, 2H), 3.26 (t, 3H), 2.99 (s, 3H), 2.70 (s, 3H), 2.62 (s, 6H).

### N-(3-((2-((4-((2-(dimethylamino)ethyl)(methyl)amino)-3-fluorophenyl)amino)-7H-pyrrolo[2,3-d]pyrimidin-4-yl)oxy)phenyl)acetamide (181a) & 7-((4-(3-acetamidophenoxy)-7H-pyrrolo[2,3-d]pyrimidin-2-yl) amino)-1,1,4-trimethyl-1,2,3,4-tetrahydroquinoxalin-1-ium (181b)

A solution of 180 (0.17 g, 0.39 mmol) and Et₃N (0.2 g, 1.98 mmol) in dichloromethane (5 mL) was stirred at 0° C. for 15 min. Then acetyl chloride (0.09 g, 1.15 mmol) was added dropwise and the mixture was stirred at 0° C. for another 1 h. The reaction mixture was quenched by saturated NaHCO₃ (10 mL) and diluted with dichloromethane (10 mL). The organic phase was washed with H₂O (5 mL*2). After concentration, the residue was purified by preparation thin liquid chromatography (dichloromethane/methanol=12/1) to give the product 181a (50.6 mg, yield 27.1%) as an off-white solid and 181b (6 mg, yield 8.4%) as a light red solid. For 181a: MS-ESI (M+Na)⁺: 501.0; For 181b: MS-ESI (M)⁺: 458.4.

### Procedure for the Synthesis of 184

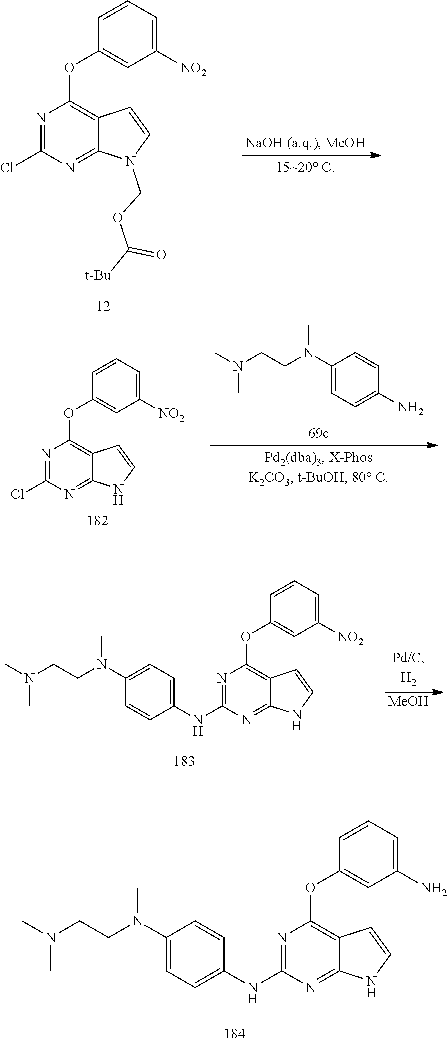

### 2-chloro-4-(3-nitrophenoxy)-7H-pyrrolo[2,3-d]pyrimidine (182)

NaOH aqueous (2.5M, 20 ml, 50 mmol) was slowly added to a solution of 12 (12 g, 29.6 mmol) in methanol (100 ml) at 15-20° C. within 10 min. The mixture was stirred for 12 h. The precipitation in the reaction mixture was collected by filtration to give the product 182 (7.2 g, yield 84%) as a white solid. MS-ESI (M+H)⁺:291.3.

### N¹-(2-(dimethylamino)ethyl)-N¹-methyl-N⁴-(4-(3-nitro phenoxy)-7H-pyrrolo[2,3-d]pyrimidin-2-yl)benzene-1,4-diamine (183)

A mixture of 69c (2.01 g, 10.40 mmol), 2-chloro-4-(3-nitrophenoxy)-7H-pyrrolo [2,3-d]pyrimidine 182 (2.88 g, 9.91 mmol), Pd₂(dba)₃ (0.89 g, w %=31%), X-Phos (0.87 g, w %=31%) and K₂CO₃ (3.89 g 28.19 mmol) in t-BuOH (60 mL) was stirred at 80° C. for 20 h. After cooling to 45° C., the mixture was filtered off and the filtrate was concentrated under reduced pressure. The residue was dissolved in ethyl acetate (60 mL), and washed with H₂O (20 mL*2), dried with Na₂SO₄. After concentration, the residue was purified by flash column chromatography (dichloromethane/methanol=15/1) to give the product 183 (1.00 g, yield 22.6%) as a gray solid.

### N¹-(4-(3-aminophenoxy)-7H-pyrrolo[2,3-d]pyrimidin-2-yl)-N⁴-(2-(dimethylamino)ethyl)-N⁴-methylbenzene-1,4-diamine (184)

To a solution of 183 (1.00 g, 2.23 mmol) in methanol (30 mL), 5% Pd/C (0.31 g, w %=31%). The mixture was stirred under H₂ at 80° C. overnight. The mixture was filtered off and the filtrate was concentrated under reduced pressure. The residue diluted with ethyl acetate (100 mL). The organic phase was washed with H₂O (30 mL*3), dried with Na₂SO₄. After concentration, the residue was purified by flash column chromatography (dichloromethane/methanol=12/1) and by preparation thin liquid chromatography (dichloromethane/methanol=12/1) to give the desired product 184 (0.051 g, yield 5.47%) as an off-white solid. MS-ESI (M+H)⁺: 418.9.

### Synthesis of Compounds of Formula II

### Procedures for the Synthesis of 189

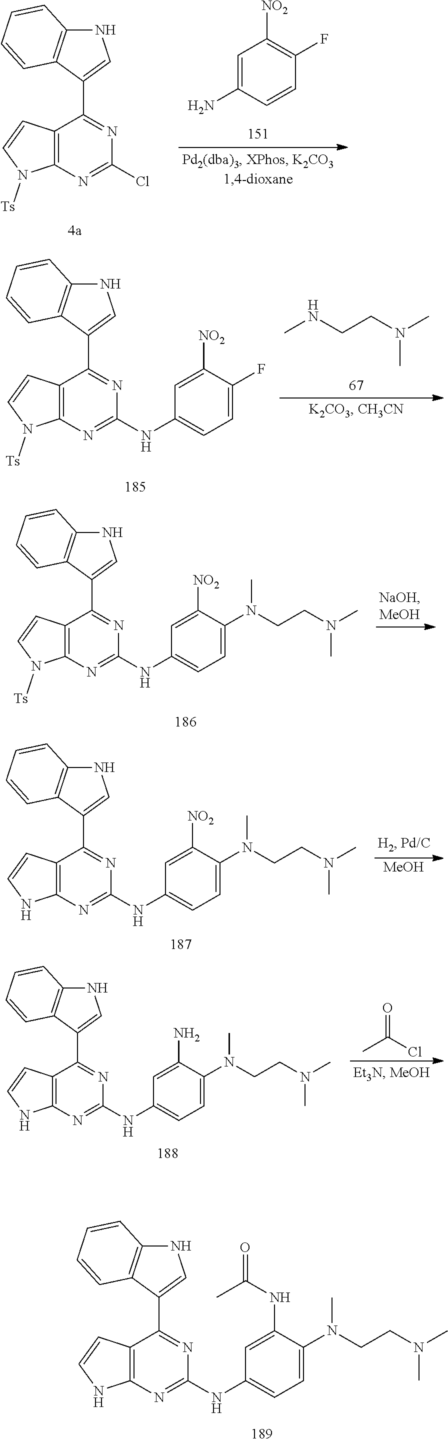

### N-(4-fluoro-3-nitrophenyl)-4-(1H-indol-3-yl)-7-tosyl-7H-pyrrolo[2,3-d]pyrimidin-2-amine (185)

4a (806 mg, 1.91 mmol), 4-fluoro-3-nitroaniline 151 (402 mg, 2.58 mmol), Pd₂(dba)₃ (210 mg, 0.23 mmol), X-phos (220 mg, 0.46 mmol), K₂CO₃ (797 mg, 5.77 mmol) and dioxane (40 mL) were sequentially added into the flask. The reaction mixture was stirred under N₂ at 80° C. for 2 h. The reaction was then cooled to room temperature, filtered through Celite® and concentrated under reduced pressure to give a residue. The residue was purified by flash column chromatography (petroleum ether/ethyl acetate=2/1˜1/5) to afford 185 (800 mg, yield 70%) as a gray solid.

### N⁴-(4-(1H-indol-3-yl)-7-tosyl-7H-pyrrolo[2,3-d]pyrimidin-2-yl)-N¹-(2-(dimethylamino)ethyl)-N¹-methyl-2-nitrobenzene-1,4-diamine (186)

N¹,N¹,N²-trimethylethane-1,2-diamine 67 (235 mg, 2.30 mmol) and K₂CO₃ (600 mg, 4.34 mmol) were added into a solution of 185 (800 mg, 1.47 mmol) in CH₃CN (20 mL) at 20° C. Then the resulting mixture was stirred at 80° C. for 2 h. The reaction was then cooled to room temperature and evaporated the solvent. Ethyl acetate (100 mL) was added to dissolve the residue and the solution was washed with water and brine. The organic layer was dried over Na₂SO₄, filtered and concentrated under reduced pressure to give the crude product of 186, which was used to next step directly without further purification.

### N⁴-(4-(1H-indol-3-yl)-7H-pyrrolo[2,3-d]pyrimidin-2-yl)-N¹-(2-(dimethylamino)ethyl)-N-methyl-2-nitrobenzene-1,4-diamine (187)

NaOH (711 mg, 17.78 mmol) was added into a solution of 186 (920 mg, 1.47 mmol, theory yield) in methanol (10 mL) at 20° C. The mixture was stirred at 40° C. for 1 h. The reaction was then cooled to room temperature. Dichloromethane (100 mL) was added to dilute the reaction and the solution was washed with saturated NH₄Cl, saturated NaHCO₃ and brine. The organic layer was then dried over Na₂SO₄, filtered and concentrated under reduced pressure to give a residue. The residue was purified by flash column chromatography (dichloromethane/methanol=20/1˜10/1) to afford 187 (150 mg, yield 21.7% over two steps) as a red solid.

### N⁴-(4-(1H-indol-3-yl)-7H-pyrrolo[2,3-d]pyrimidin-2-yl)-N¹-(2-(dimethylamino)ethyl)-N¹-methylbenzene-1,2,4-triamine (188)

Pd/C (5% w/w, 55 mg) was added into a solution of 187 (150 mg, 0.32 mmol) in methanol (20 mL) at 20° C. Then mixture was stirred at 40° C. under H₂ for 4 h. The reaction was then cooled to room temperature, and filtered through Celite®. The filtrate was concentrated under reduced pressure to give a residue. The residue was purified by flash column chromatography (dichloromethane/methanol=10/1, 1% Et₃N) to afford 188 (32 mg, yield 22.8%) as a slight yellow solid. MS-ESI (M+H)⁺: 441.5.

### N-(5-((4-(1H-indol-3-yl)-7H-pyrrolo[2,3-d]pyrimidin-2-yl)amino)-2-((2-(dimethylamino)ethyl)(methyl)amino)phenyl)acetamide (189)

CH₃COCl (50 mg, 0.64 mmol) was added into a solution of 188 (30 mg, 0.068 mmol) and Et₃N (50 mg, 0.49 mmol) in methanol (10 mL) at 20° C. The mixture was stirred for 2.5 h. Then the reaction was concentrated under reduced pressure to give a residue. The residue was purified by preparative thin layer chromatography (dichloromethane/methanol=10/1, 1% Et₃N) to afford 189 (30 mg, yield 91.3%) as a white solid. ¹H NMR (500 MHz, DMSO) δ 9.55 (s, 1H), 9.46 (s, 1H), 8.75 (d, J=8.0 Hz, 1H), 8.38 (d, J=3.9 Hz, 1H), 8.34 (s, 1H), 8.25 (t, J=5.4 Hz, 1H), 7.60-7.48 (m, 2H), 7.31 (dt, J=8.2, 5.5 Hz, 1H), 7.25-7.16 (m, 2H), 3.28-3.13 (m, 4H), 2.73 (s, 6H), 2.56 (s, 3H), 2.22 (s, 3H). MS-ESI (M+H)⁺: 483.4.

### Procedure for the Synthesis of 190

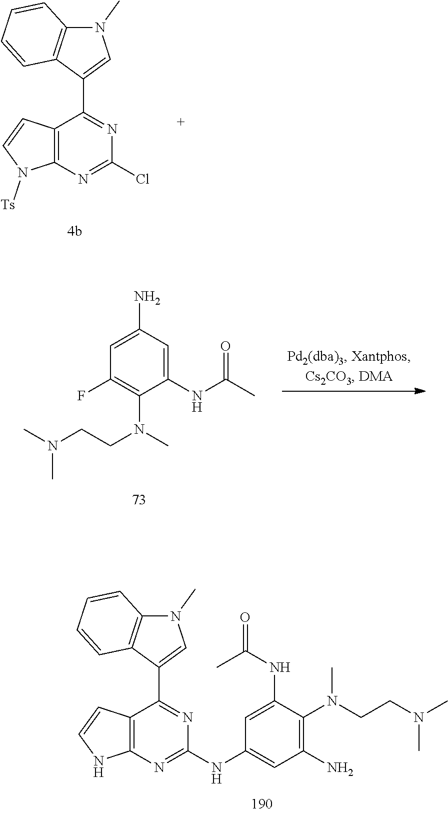 [SMILES: Cc1ccc(S(=O)(=O)n2ccc3c(-c4cn(C)c5ccccc45)nc(Cl)nc32)cc1 ; CC(=O)Nc1cc(N)cc(F)c1N(C)CCN(C)C ; CC(=O)Nc1cc(Nc2nc(-c3cn(C)c4ccccc34)c3cc[nH]c3n2)cc(N)c1N(C)CCN(C)C]

### N-(2-((2-(dimethylamino)ethyl)(methyl)amino)-3-fluoro-5-((4-(1-methyl-1H-indol-3-yl)-7H-pyrrolo[2,3-d]pyrimidin-2-yl)amino)phenyl)acetamide (190)

To a solution of 73 (200 mg, 0.75 mmol) and 2-chloro-4-(1-methyl-1H-indol-3-yl)-7-tosyl-7H-pyrrolo[2,3-d]pyrimidine 4b (310 mg, 0.71 mmol) in DMA (5 mL) were added Cs₂CO₃ (694 mg, 2.1 mmol), Pd₂(dba)₃ (65 mg, 0.71 mmol) and Xant-phos (42 mg, 0.71 mmol). The mixture was stirred at 145° C. under Ar overnight. Six batches were set up in parallel and combined to do the workup. After cooling to room temperature, the mixture was diluted with ethyl acetate (500 mL). The organic phase was washed with H₂O (250 mL) and brine (250 mL*2), dried with Na₂SO₄. After concentration, the residue was purified by Pre-HPLC (MeCN/0.05% formic acid in water from 0 to 100%)) to give the title product as FA salt, which was neutralized with saturated sodium carbonate to give 190 as a yellow solid (147.93 mg, yield 6.8%). MS-ESI (M+H)⁺: 515.2. ¹H NMR (400 MHz, DMSO-d6) δ 11.51 (s, 1H), 10.28 (br, 1H), 9.41 (s, 1H), 8.96 (d, J=7.6 Hz, 1H), 8.45 (s, 1H), 8.20 (s, 1H), 8.02 (d, J=7.6 Hz, 1H), 7.53 (d, J=7.2 Hz, 1H), 7.31-7.27 (m, 1H), 7.23-7.21 (m, 2H), 6.90 (s, 1H), 3.96 (s, 3H), 3.05 (s, 2H), 2.76 (s, 3H), 2.31 (br, 8H), 2.12 (s, 3H).

### Procedure for the Synthesis of 192

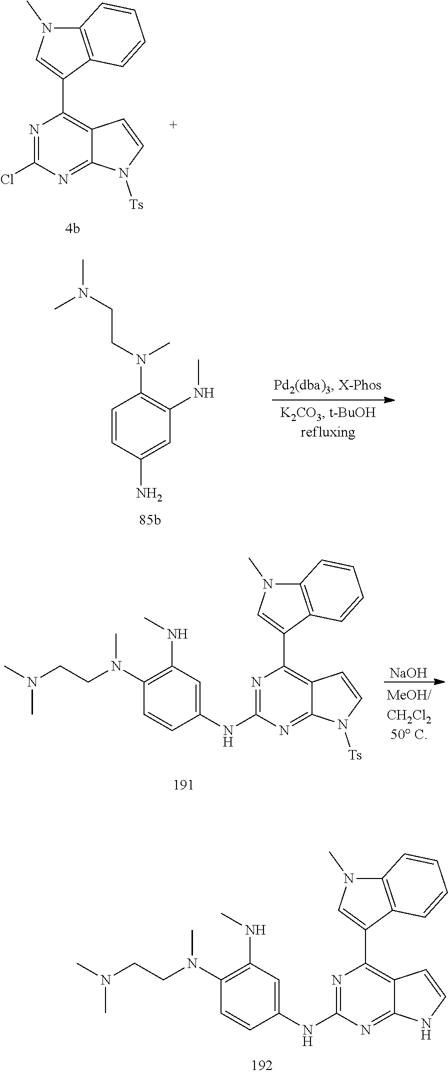

### N¹-(2-(dimethylamino)ethyl)-N¹,N²-dimethyl-N⁴-(4-(1-methyl-1H-indol-3-yl)-7-tosyl-7H-pyrrolo[2,3-d]pyrimidin-2-yl)benzene-1,2,4-triamine (191)

A mixture of intermediate 4b (560.1 mg, 1.28 mmol), intermediate 85b (260 mg, 1.17 mmol), Pd₂(dba)₃ (112.6 mg, 0.12 mmol), X-Phos (108.9 mg, 0.23 mmol) and K₂CO₃ (496.8 mg, 3.59 mmol) in t-BuOH (30 mL) was stirred under N₂ at refluxing temperature for 17 h. After cooling to room temperature, the reaction mixture was filtered through Celite® and the filtrates were concentrated under reduced pressure to dryness. The crude was purified by flash column chromatography (dichloromethane/methanol=20/1) to give the title compound 191 (360 mg, yield 49.4%) as a brown oil.

### N¹-(2-(dimethylamino)ethyl)-N¹,N²-dimethyl-N⁴-(4-(1-methyl-1H-indol-3-yl)-7H-pyrrolo[2,3-d]pyrimidin-2-yl)benzene-1,2,4-triamine (192)

NaOH (296.8 mg, 7.42 mmol) was added into a solution of compound 191 (300 mg, 0.48 mmol) in methanol (4 mL) and dichloromethane (1 mL). The reaction mixture was stirred at 50° C. for 4 h. Solvents were removed in vacuo followed by addition of acetic acid. The mixture was extracted with dichloromethane. The organic layers were separated, dried over anhydrous Na₂SO₄ and then concentrated to dryness. The crude was purified by preparative thin layer chromatography to give the title compound 192 (56 mg, yield 24.8%) as a light-yellow solid.

### Procedure for the Synthesis of 194

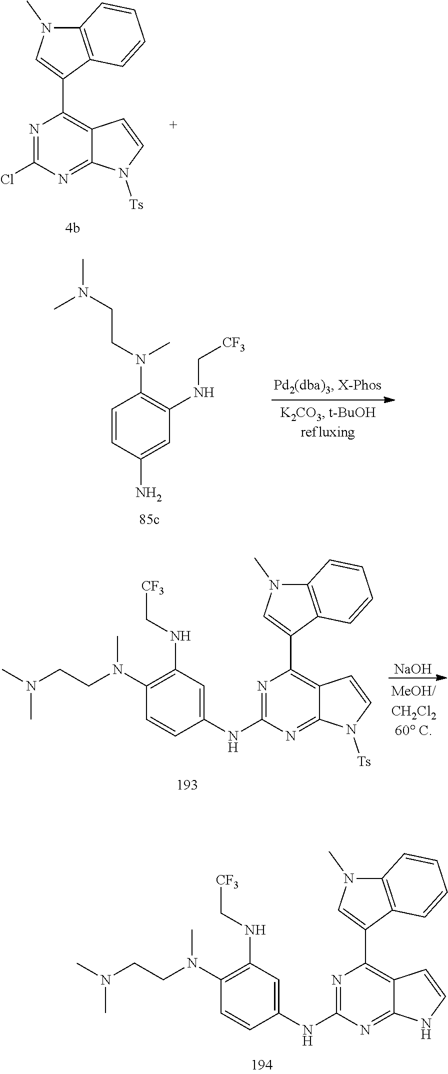

### N¹-(2-(dimethylamino)ethyl)-N¹-methyl-N⁴-(4-(1-methyl-1H-indol-3-yl)-7-tosyl-7H-pyrrolo[2,3-d]pyrimidin-2-yl)-N²-(2,2,2-trifluoroethyl)benzene-1,2,4-triamine (193)

A mixture of intermediate 4b (513.4 mg, 1.18 mmol), intermediate 85c (210 mg, 0.72 mmol), Pd₂(dba)₃ (118.3 mg, 0.13 mmol), X-Phos (121.6 mg, 0.26 mmol) and K₂CO₃ (510.2 mg, 3.69 mmol) in t-BuOH (30 mL) was stirred under N₂ at refluxing temperature for 15.5 h. After cooling to room temperature, the reaction mixture was filtered through Celite®. The filter cake was washed with methanol/ethyl acetate and the filtrates were concentrated under reduced pressure to dryness. The crude was purified by flash column chromatography (dichloromethane/methanol=30/1) to give the title compound 193 (315 mg, yield 63.0%) as a brown oil.

### N¹-(2-(dimethylamino)ethyl)-N¹-methyl-N⁴-(4-(1-methyl-1H-indol-3-yl)-7H-pyrrolo[2,3-d]pyrimidin-2-yl)-N²-(2,2,2-trifluoroethyl)benzene-1,2,4-triamine (194)

NaOH (315 mg, 0.46 mmol) was added into a solution of compound 193 (54 mg, 1.35 mmol) in methanol (5 mL) and dichloromethane (5 mL). The reaction mixture was stirred at 60° C. for 2.5 h. Solvents were removed in vacuo. The crude was purified by flash column chromatography (dichloromethane/methanol=20/1) to give the title compound 194 (20 mg, yield 8.2%) as a light-yellow solid.

### Procedure for the Syntheses of 195a and 195b

### N-(2-((2-(dimethylamino)ethyl)(ethyl)amino)-3-fluoro-5-((4-(1-methyl-1H-indol-3-yl)-7H-pyrrolo[2,3-d]pyrimidin-2-yl)amino)phenyl)acetamide (195a) & N-(2-((2-(dimethylamino)ethyl)(ethyl)amino)-3-fluoro-5-((7-methyl-4-(1-methyl-1H-indol-3-yl)-7H-pyrrolo[2,3-d]pyrimidin-2-yl)amino)phenyl)acetamide (195b)

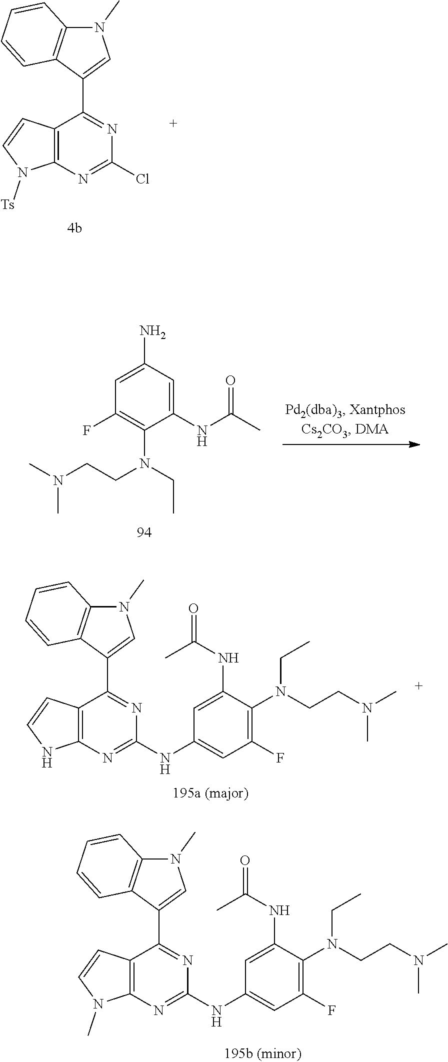 [SMILES: Cc1ccc(S(=O)(=O)n2ccc3c(-c4cn(C)c5ccccc45)nc(Cl)nc32)cc1 ; CCN(CCN(C)C)c1c(F)cc(N)cc1NC(C)=O ; CCN(CCN(C)C)c1c(F)cc(Nc2nc(-c3cn(C)c4ccccc34)c3cc[nH]c3n2)cc1NC(C)=O ; CCN(CCN(C)C)c1c(F)cc(Nc2nc(-c3cn(C)c4ccccc34)c3ccn(C)c3n2)cc1NC(C)=O]

Synthesis of 195a was similar to that of 190. The residue was purified by Pre-HPLC (MeCN/0.05% NH₄OH in water from 0 to 100%) to give 195a as a light red solid (67 mg, yield 6.9%). MS-ESI (M+H)⁺: 529.2; ¹H NMR (400 MHz, DMSO-d6) δ 11.50 (s, 1H), 10.38 (s, 1H), 9.39 (s, 1H), 8.94 (d, J=7.6 Hz, 1H), 8.44 (s, 1H), 8.16 (s, 1H), 7.98 (d, J=7.6 Hz, 1H), 7.52 (d, J=7.2 Hz, 1H), 7.39-7.37 (m, 1H), 7.30-7.26 (m, 1H), 7.21-7.20 (m, 1H), 6.89 (d, J=7.2 Hz, 1H), 3.95 (s, 3H), 3.08-2.99 (m, 4H), 2.22-2.17 (m, 8H), 2.07 (s, 3H), 0.89 (t, J=7.6 Hz, 3H).

A small amount of byproduct 195b (15 mg, yield 1.6%) was also isolated.

### Procedure for the Synthesis of 197

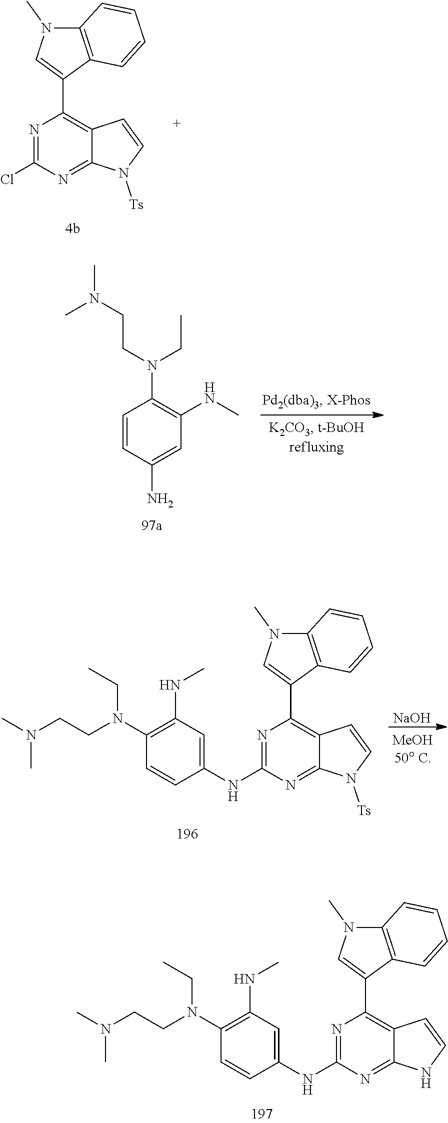

### N¹-(2-(dimethylamino)ethyl)-N¹-ethyl-N²-methyl-N⁴-(4-(1-methyl-1H-indol-3-yl)-7-tosyl-7H-pyrrolo[2,3-d]pyrimidin-2-yl)benzene-1,2,4-triamine (196)

A mixture of 2-chloro-4-(1-methyl-1H-indol-3-yl)-7-tosyl-7H-pyrrolo[2,3-d]pyrimidine 4b (731 mg, 1.67 mmol), N¹-(2-(dimethylamino)ethyl)-N¹-ethyl-N²-methylbenzene-1,2,4-triamine 97a (360 mg, 1.52 mmol), Pd₂(dba)₃ (145.0 mg, 0.16 mmol), X-Phos (148.0 mg, 0.31 mmol) and K₂CO₃ (661 mg, 4.78 mmol) in t-BuOH (30 mL) was stirred under N₂ at refluxing temperature for 17 h. After cooling to room temperature, the reaction mixtures was filtered through Celite® and the filtrates were concentrated under reduced pressure to dryness. The crude was purified by flash column chromatography (dichloromethane/methanol=25/1) to give the title compound 196 (410 mg, yield 42.4%) as a brown solid.

### N¹-(2-(dimethylamino)ethyl)-N¹-ethyl-N²-methyl-N⁴-(4-(1-methyl-1H-indol-3-yl)-7H-pyrrolo[2,3-d]pyrimidin-2-yl)benzene-1,2,4-triamine (197)

NaOH (210.4 mg, 5.26 mmol) was added into a solution of compound 196 (150 mg, 0.24 mmol) in methanol (5 mL). The reaction mixture was stirred at 50° C. for 2 h. Solvent was removed in vacuo followed by addition of aqueous NH₄Cl. The mixture was extracted with dichloromethane. The organic layers were separated, dried over anhydrous Na₂SO₄ and then concentrated to dryness. The crude was purified by preparative thin layer chromatography to give the title compound 197 (23 mg, yield 20.2%) as a light-yellow solid.

### Procedure for the Synthesis of 201

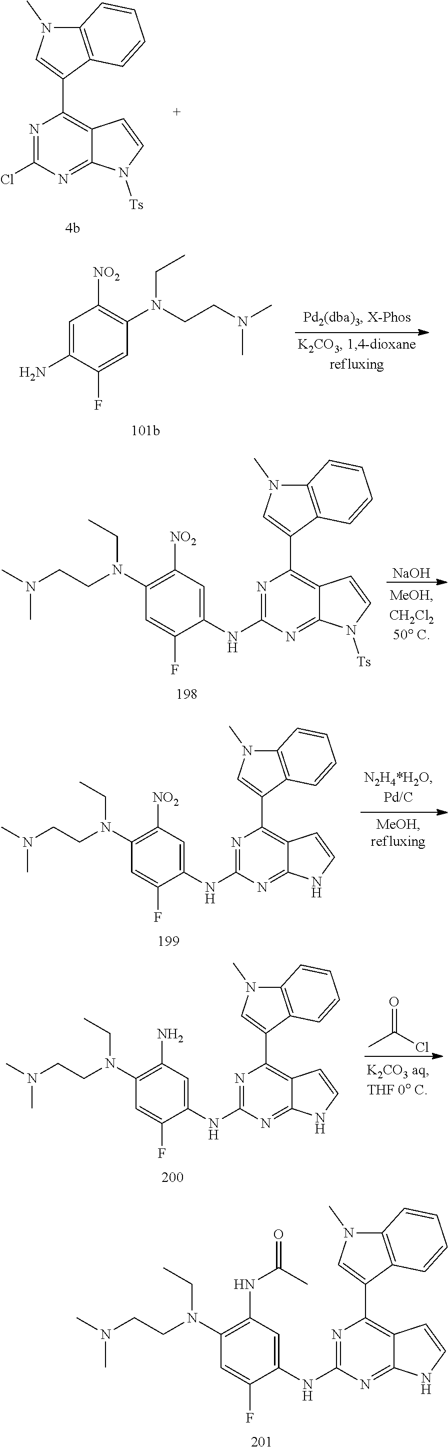

### N¹-(2-(dimethylamino)ethyl)-N¹-ethyl-5-fluoro-N⁴-(4-(1-methyl-1H-indol-3-yl)-7-tosyl-7H-pyrrolo[2,3-d]pyrimidin-2-yl)-2-nitrobenzene-1,4-diamine (198)

A mixture of intermediate 4b (504 mg, 1.15 mmol), intermediate 101b (308 mg, 1.23 mmol), Pd₂(dba)₃ (115 mg, 0.13 mmol), X-Phos (125 mg, 0.26 mmol) and K₂CO₃ (500 mg, 3.62 mmol) in t-BuOH (30 mL) was stirred under N₂ at refluxing temperature for 19 h. After cooling to room temperature, the reaction mixture was filtered through Celite® and the filtrates were concentrated under reduced pressure to dryness. The crude was purified by flash column chromatography (dichloromethane/methanol=20/1) to give the title compound 198 (288 mg, yield 37.2%) as a brown oil.

### N¹-(2-(dimethylamino)ethyl)-N¹-ethyl-5-fluoro-N⁴-(4-(1-methyl-1H-indol-3-yl)-7H-pyrrolo[2,3-d]pyrimidin-2-yl)-2-nitrobenzene-1,4-diamine (199)

NaOH (85 mg, 2.13 mmol) was added into a solution of compound 198 (280 mg, 0.42 mmol) in methanol (10 mL) and dichloromethane (2 mL). The reaction mixture was stirred at 50° C. overnight. Solvents were removed in vacuo followed by addition of dichloromethane (20 mL) and H₂O (10 mL). The water phase was neutralized to pH 7 with acetic acid, and then extracted with dichloromethane. The organic layers were separated, dried over anhydrous Na₂SO₄ and then concentrated to give the title compound 199 (272 mg) as a red solid, which was used in the next step without further purification.

### N¹-(2-(dimethylamino)ethyl)-N¹-ethyl-5-fluoro-N⁴-(4-(1-methyl-1H-indol-3-yl)-7H-pyrrolo[2,3-d]pyrimidin-2-yl)benzene-1,2,4-triamine (200)

The mixture of compound 199 (272 mg, 0.53 mmol), Pd/C (5%, 156 mg, 0.07 mmol) and hydrazine hydrate (80%, 5 ml, 80 mmol) in methanol (20 ml) was stirred at refluxing temperature for 2.5 h. The mixture was filtered, concentrated and then extracted with ethyl acetate (50 ml*3). The organic layers were combined, washed with brine (100 ml*3), and then concentrated under reduced pressure to give the compound 200 (205 mg) as a yellow solid, which was used in the next step without further purification.

### N-(2-((2-(dimethylamino)ethyl)(ethyl)amino)-4-fluoro-5-((4-(1-methyl-1H-indol-3-yl)-7H-pyrrolo[2,3-d]pyrimidin-2-yl)amino)phenyl)acetamide (201)

To a solution of 200 (205 mg, 0.42 mmol) in tetrahydrofuran (10 mL) and saturated K₂CO₃ aqueous (1 mL) in was added acetyl chloride (120 mg, 1.52 mmol) dropwise at 0° C. The reaction was stirred at room temperature for 1 h, and then quenched with H₂O. The mixture was extracted with ethyl acetate (10 mL). The organic layer was washed with brine, dried over Na₂SO₄ and concentrated. The residue was purified by preparative thin layer chromatography (dichloromethane/methanol=100/7) to afford the title compound 201 (9 mg, 4.1% yield over three steps) as a slight yellow solid.

### General Procedures for the Syntheses of 204 and 205a-c

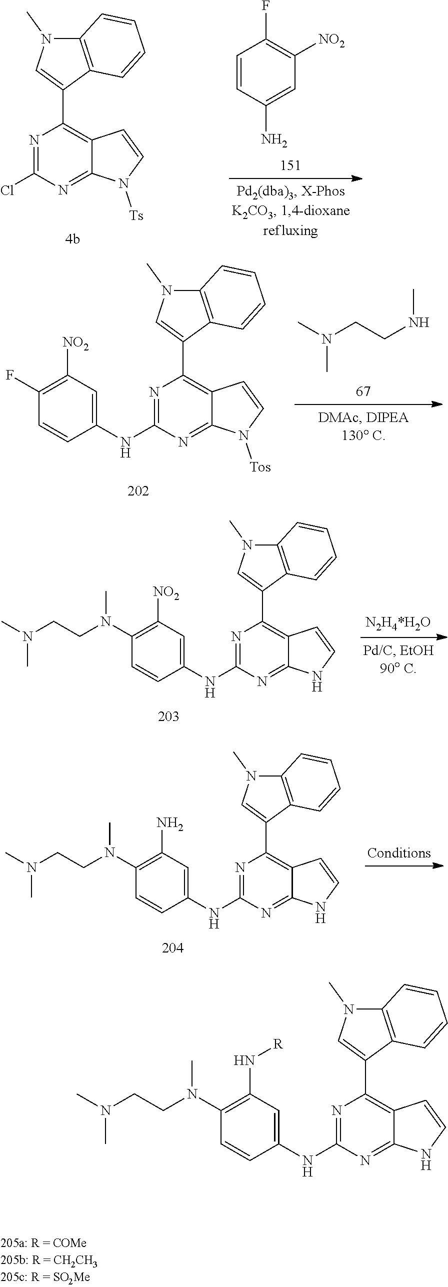

### N-(4-fluoro-3-nitrophenyl)-4-(1-methyl-1H-indol-3-yl)-7-tosyl-7H-pyrrolo[2,3-d]pyrimidin-2-amine (202)

X-Phos (0.30 g, 0.63 mmol), Pd₂(dba)₃ (0.31 g, 0.34 mmol), K₂CO₃ (2.12 g, 15.33 mmol) and compound 4b (3.02 g, 6.91 mmol) was added to a stirred solution of 4-fluoro-3-nitroaniline 151 (0.96 g, 6.15 mmol) in 1,4-dioxane (50 ml). The resulting mixture was heated at 110° C. for 4 h. After cooling to room temperature, the mixture was filtered and the filtrate was concentrated. The residue was stirred with ethyl acetate (100 ml) at 60° C. for 2 h. After cooling to the room temperature, the mixture was filtered and the filter cake was dried in vacuo to give the title product 202 (3.0 g, yield 87.73%). MS-ESI (M+H)⁺:557.6.

### N¹-(2-(dimethylamino)ethyl)-N¹-methyl-N⁴-(4-(1-methyl-1H-indol-3-yl)-7H-pyrrolo[2,3-d]pyrimidin-2-yl)-2-nitrobenzene-1,4-diamine (203)

The mixture of compound 202 (3.0 g, 5.38 mmol), N′,N′,N-Trimethylethane-1,2-diamine 67 (1.31 g, 12.84 mmol) and N, N-diisopropylethylamine (2.10 g, 16.27 mmol) in N,N-dimethyl acetamide (20 mL) was stirred at 140° C. for 4 h. The reaction mixture was cooled down and quenched with water (50 ml). The mixture was extracted with ethyl acetate (50 ml*3). The organic layers were combined, washed with brine (100 ml*3), and then concentrated under reduced pressure to give the tittle compound 203 (2.3 g, yield 88.28%), which was used for next step without further purification. MS-ESI (M+H)⁺:485.6.

### N¹-(2-(dimethylamino)ethyl)-N¹-methyl-N⁴-(4-(1-methyl-1H-indol-3-yl)-7H-pyrrolo[2,3-d]pyrimidin-2-yl)benzene-1,2,4-triamine (204)

The mixture of compound 203 (2.3 g, 4.74 mmol), Pd/C (5%, 0.11 g, 0.05 mmol) and hydrazine hydrate (80%, 15 ml) in methanol (30 ml) was stirred at refluxing temperature for 5 h. The mixture was filtered, concentrated and then extracted with ethyl acetate (50 ml*3). The organic layers were combined, washed with brine (100 ml*3), and then concentrated under reduced pressure. The residue was purified by flash column chromatography (dichloromethane/methanol=20/1) to give the compound 204 (1.0 g, yield 46.44%). MS-ESI (M+H)⁺: 455.5. ¹H NMR (500 MHz, DMSO-d6) δ 11.38 (s, 1H), 8.93 (d, J=7.9 Hz, 1H), 8.72 (s, 1H), 8.41 (s, 1H), 7.54-7.46 (m, 1H), 7.27 (t, J=7.5 Hz, 1H), 7.20 (t, J=7.5 Hz, 1H), 7.17-7.12 (m, 2H), 7.11 (d, J=2.4 Hz, 1H), 6.91 (d, J=8.5 Hz, 1H), 6.86 (d, J=5.3 Hz, 1H), 3.94 (s, 3H), 2.89 (s, 2H), 2.59 (s, 3H), 2.42 (t, J=6.5 Hz, 2H), 2.22 (s, 6H).

### N-(2-((2-(dimethylamino)ethyl)(methyl)amino)-5-((4-(1-methyl-1H-indol-3-yl)-7H-pyrrolo[2,3-d]pyrimidin-2-yl)amino)phenyl)acetamide (205a)

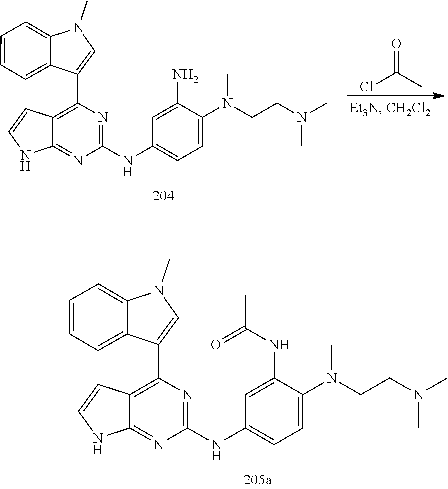

To the mixture of compound 204 (0.20 g, 0.44 mmol) and trimethylamine (0.10 g, 0.99 mmol) in dichloromethane (10 ml), acetyl chloride (0.05 g, 0.64 mmol) was added at 0° C. And then the mixture was stirred for a further 0.5 h. Then the mixture was concentrated and diluted with ethyl acetate (50 ml). The organic layers were combined, washed with brine (50 ml*3), and then concentrated under reduced pressure. The residue was purified by preparative thin layer chromatography (dichloromethane/methanol=20/1) to give the compound 205a (60 mg, yield 27.47%). MS-ESI (M+H)⁺:497.8; ¹H NMR (500 MHz, DMSO-d6) δ 11.43 (s, 1H), 9.08 (s, 1H), 8.91 (d, J=7.9 Hz, 1H), 8.43 (s, 1H), 8.33 (s, 1H), 7.76 (dd, J=8.7, 2.5 Hz, 1H), 7.52 (d, J=8.2 Hz, 1H), 7.27 (t, J=8.1 Hz, 1H), 7.20 (t, J=7.9 Hz, 1H), 7.18-7.14 (m, 2H), 6.87 (dd, J=3.6, 1.8 Hz, 1H), 5.77 (s, 0H), 3.95 (s, 3H), 2.67 (s, 6H), 2.57 (s, 3H), 2.51-2.50 (m, 4H), 2.23 (s, 3H).

### N¹-(2-(dimethylamino)ethyl)-N²-ethyl-N1-methyl-N⁴-(4-(1-methyl-1H-indol-3-yl)-7H-pyrrolo[2,3-d]pyrimidin-2-yl)benzene-1,2,4-triamine (205b)

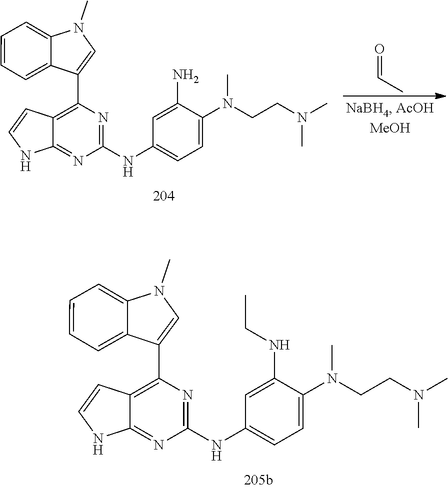

To the mixture of compound 204 (0.15 g, 0.33 mmol), acetic acid (0.1 ml) and acetaldehyde aqueous (3 ml) in methanol (6 ml), NaBH₄ (0.10 g, 2.63 mmol) was slowly added at room temperature. The mixture was stirred for 1.5 h. Ethyl acetate (50 ml) and water (50 ml) was added to the mixture. The organic layer was washed by saturated NaHCO₃ aqueous solution (50 ml), and then concentrated under reduced pressure. The residue was purified by flash column chromatography (dichloromethane/methanol=20/1) to give the compound 205b (110 mg, yield 69.11%). MS-ESI (M+H)⁺:483.29. ¹H NMR (500 MHz, DMSO-d6) δ 11.38 (s, 1H), 8.93 (d, J=7.9 Hz, 1H), 8.41 (s, 1H), 7.52 (d, J=8.2 Hz, 1H), 7.30-7.25 (m, 1H), 7.25-7.17 (m, 2H), 7.16 (dd, J=3.5, 2.3 Hz, 1H), 7.10 (d, J=2.3 Hz, 1H), 6.86 (dd, J=3.6, 1.8 Hz, 1H), 3.95 (s, 3H), 3.13 (q, J=7.1 Hz, 2H), 2.92 (q, J=7.0 Hz, 4H), 2.55 (s, 3H), 2.42 (s, 6H), 1.14 (t, J=7.3 Hz, 3H).

### N-(2-((2-(dimethylamino)ethyl)(methyl)amino)-5-((4-(1-methyl-1H-indol-3-yl)-7H-pyrrolo[2,3-d]pyrimidin-2-yl)amino)phenyl)methanesulfonamide (205c)

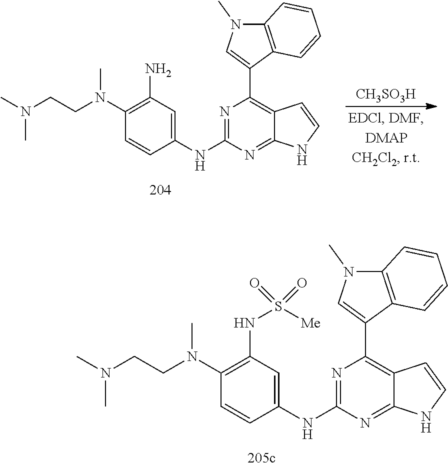

A mixture of 1-(3-Dimethylaminopropyl)-3-ethylcarbodiimide (600 mg, 3.9 mmol), 4-Dimethylaminopyridine (20 mg, 0.16 mmol), methanesulfonic acid (200 mg, 2.1 mmol) in dichloromethane (20 ml) was stirred at room temperature for 0.5 h. Then 204 (1.00 g, 2.2 mmol) and N,N-dimethylformamide (1 drop) were added and the resulting mixture was stirred for 2 h before quenching with water. The organic layer was separated, washed with water, and concentrated to dryness. The crude was purified by preparative thin layer chromatography (methanol/dichloromethane=1/20) to give the title compound 205c (113 mg, yield 9.7%) as a brown solid.

### Procedure for the Synthesis of 206

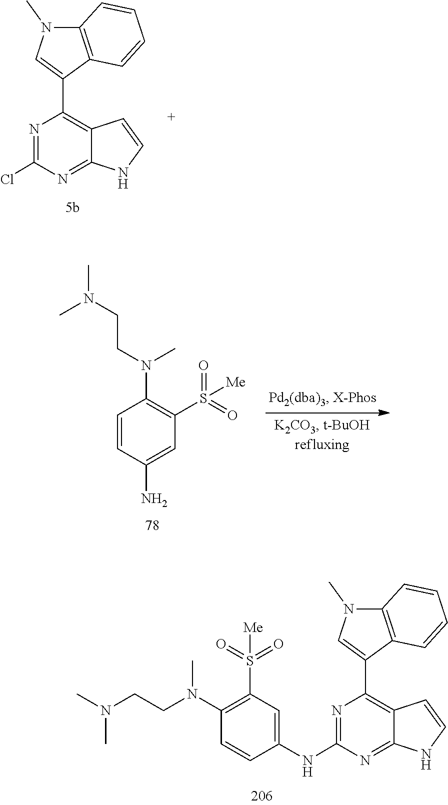 [SMILES: Cn1cc(-c2nc(Cl)nc3[nH]ccc23)c2ccccc21 ; CN(C)CCN(C)c1ccc(N)cc1S(C)(=O)=O ; CN(C)CCN(C)c1ccc(Nc2nc(-c3cn(C)c4ccccc34)c3cc[nH]c3n2)cc1S(C)(=O)=O]

### N¹-(2-(dimethylamino)ethyl)-N¹-methyl-N⁴-(4-(1-methyl-1H-indol-3-yl)-7H-pyrrolo[2,3-d]pyrimidin-2-yl)-2-(methylsulfonyl)benzene-1,4-diamine (206)

A mixture of 5b (0.5 g, 1.8 mmol), 78 (0.479 g, 1.8 mmol), Pd₂(dba)₃ (160 mg, 0.17 mmol), X-Phos (160 mg, 0.34 mmol) and K₂CO₃ (0.73 g, 5.3 mmol) in t-BuOH (20 mL) was stirred under N₂ at refluxing temperature for 8 h. After cooling to room temperature, the reaction mixtures was filtered through Celite® and the filtrates were concentrated under reduced pressure to dryness. The crude was purified by preparative thin layer chromatography to give the title compound 206 (92.5 mg, yield 10.1%) as a light-yellow solid.

### Procedure for the Synthesis of 207

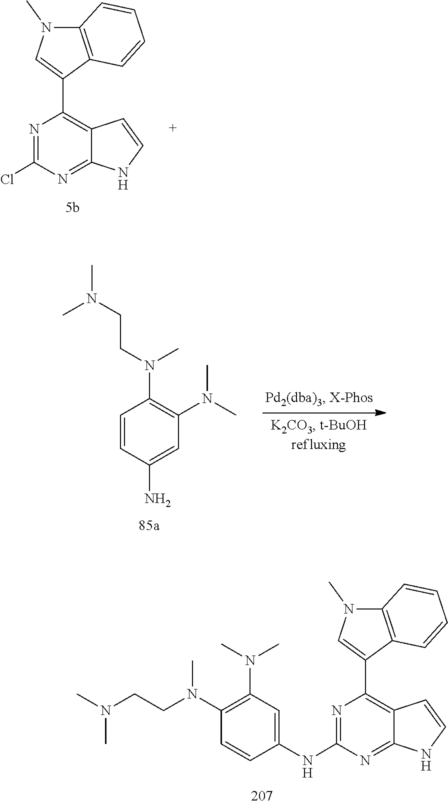

### N¹-(2-(dimethylamino)ethyl)-N¹,N²,N²-trimethyl-N⁴-(4-(1-methyl-1H-indol-3-yl)-7H-pyrrolo[2,3-d]pyrimidin-2-yl)benzene-1,2,4-triamine (207)

A mixture of 5b (250.4 mg, 0.89 mmol), 85a (190 mg, 0.80 mmol), Pd₂(dba)₃ (73.7 mg, 0.08 mmol), X-Phos (78.1 mg, 0.16 mmol) and K₂CO₃ (334.0 mg, 2.42 mmol) in t-BuOH (40 mL) was stirred under N₂ at refluxing temperature for 17 h. After cooling to room temperature, the reaction mixtures was filtered through Celite® and the filtrates were concentrated under reduced pressure to dryness. The crude was purified by preparative thin layer chromatography to give the title compound 207 (22 mg, yield 5.7%) as a light-yellow solid.

### Procedure for the Synthesis of 208

### N¹-(2-(dimethylamino)ethyl)-N¹-ethyl-2-fluoro-N⁴-(4-(1-methyl-1H-indol-3-yl)-7H-pyrrolo[2,3-d]pyrimidin-2-yl)benzene-1,4-diamine (208)

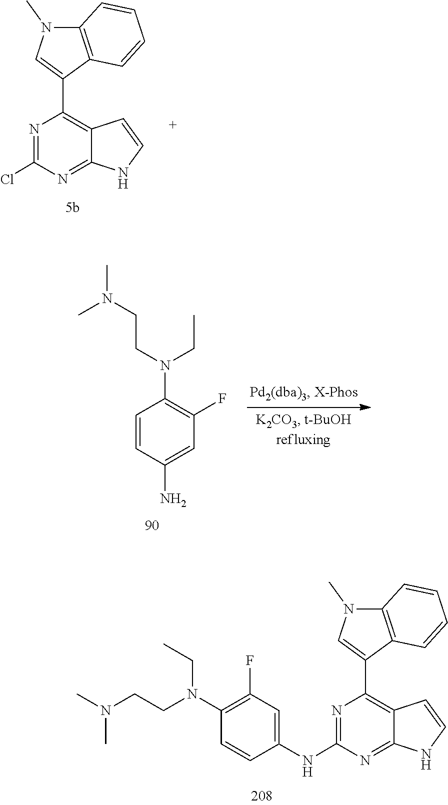 [SMILES: Cn1cc(-c2nc(Cl)nc3[nH]ccc23)c2ccccc21 ; CCN(CCN(C)C)c1ccc(N)cc1F ; CCN(CCN(C)C)c1ccc(Nc2nc(-c3cn(C)c4ccccc34)c3cc[nH]c3n2)cc1F]

A mixture of 5b (0.3215 g, 1.1 mmol), 90 (0.22 g, 0.98 mmol), Pd₂(dba)₃ (0.0910 g, 0.1 mmol), X-Phos (0.1020 g, 0.2 mmol) and K₂CO₃ (0.4350 g, 3.1 mmol) in t-BuOH (40 mL) was stirred under N₂ at refluxing temperature for 20 h. After cooling to room temperature, the reaction mixtures was filtered through Celite® and the filtrates were concentrated under reduced pressure to dryness. The crude was purified by flash column chromatography (eluting with methanol/dichloromethane, from 1/30 to 1/10, v/v) to give the title compound 208 (36 mg, yield 7.8%) as a brown solid. ¹H NMR (500 MHz, DMSO) δ 11.49 (s, 1H), 9.17 (s, 1H), 8.88 (d, J=8.0 Hz, 1H), 8.43 (s, 1H), 7.98 (dd, J=15.5, 2.4 Hz, 1H), 7.54 (d, J=8.2 Hz, 1H), 7.47 (dd, J=8.7, 2.1 Hz, 1H), 7.33-7.26 (m, 1H), 7.24-7.17 (m, 2H), 7.08-7.00 (m, 1H), 6.88 (dd, J=3.6, 1.8 Hz, 1H), 3.95 (s, 3H), 3.18 (t, J=7.0 Hz, 2H), 3.11 (q, J=7.0 Hz, 2H), 2.46 (t, J=6.7 Hz, 2H), 2.25 (s, 6H), 0.99 (t, J=7.0 Hz, 3H). LC-MS: m/z 472.9 [M+H]⁺.

### General Procedures for the Syntheses of 211, 212a-f

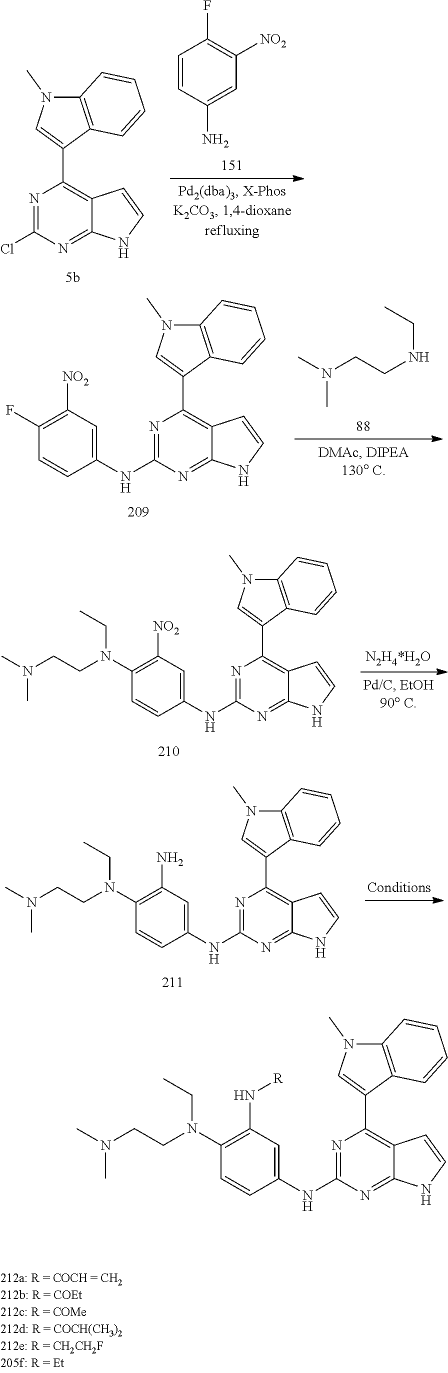

### N-(4-fluoro-3-nitrophenyl)-4-(1-methyl-1H-indol-3-yl)-7H-pyrrolo[2,3-d]pyrimidin-2-amine (209)

A mixture of 5a (4.51 g, 16.0 mmol), 4-fluoro-3-nitroaniline 151 (2.51 g, 16.1 mmol), Pd₂(dba)₃ (1.33 g, 1.5 mmol), X-Phos (1.33 g, 2.8 mmol) and K₂CO₃ (6.0 g, 43.4 mmol) in 1,4-dioxane (45 mL) was stirred under N₂ at 85° C. for 4 h. After cooling to room temperature, the reaction mixtures was filtered through Celite® and the filtrates were concentrated under reduced pressure until the volume of the rest was about 25 ml. The residual solution was stirred for 30 min for precipitation followed by addition of heptane (15 ml). The resulting mixture was stirred at room temperature for 30 min and at 0° C. for another 30 min. The solid was collected by filtration and dried to give the title compound 209 (5.3 g, yield 82.6%) as a yellow solid.

### N¹-(2-(dimethylamino)ethyl)-N¹-ethyl-N⁴-(4-(1-methyl-1H-indol-3-yl)-7H-pyrrolo[2,3-d]pyrimidin-2-yl)-2-nitrobenzene-1,4-diamine (210)

N,N-diisopropyl ethylamine (2.3 g, 17.8 mmol) was added to a solution of 209 (4.01 g, 10.0 mmol) and N′-ethyl-N,N-dimethyl ethylenediamine 88 (2.0 g, 17.2 mmol) in N,N-dimethylacetamide (35 ml) at room temperature. The mixture was stirred at 130° C. for 4.5 h then cooled to room temperature. Water (60 ml) was added and the mixture was extracted with ethyl acetate (100 mL). The organic layer was separated, washed with water for 3 times, dried over Na₂SO₄, filtered and concentrated to dryness. The crude was purified by flash column chromatography (eluting with methanol/dichloromethane, from 1/10 to 1/5, v/v) to give the title compound 210 (3.5 g, yield 70.4%) as a yellow solid.

### N¹-(2-(dimethylamino)ethyl)-N¹-ethyl-N⁴-(4-(1-methyl-1H-indol-3-yl)-7H-pyrrolo[2,3-d]pyrimidin-2-yl)benzene-1,2,4-triamine (211)

A mixture of 210 (3.40 g, 6.8 mmol), Pd/C (5% on activated carbon, 0.40 g, 0.19 mmol) and hydrazine hydrate (80%, 15 ml, 247.2 mmol) in ethanol (35 ml) was heated at 80˜90° C. for 4 h. After cooling to room temperature, the reaction mixtures was filtered through Celite® and the filtrates were concentrated to remove ethanol. The residual was extracted with ethyl acetate. The organic layer was separated, washed with water, dried over Na₂SO₄, filtered and concentrated to dryness. The crude was purified by flash column chromatography (eluting with methanol/dichloromethane, from 1/20 to 1/10, v/v) to give the title compound 211 (2.3 g, yield 72.0%) as an oil. LC-MS: m/z 469.4 [M+H]⁺. ¹H NMR (500 MHz, DMSO) δ 11.35 (s, 1H), 8.91 (d, J=8.0 Hz, 1H), 8.72 (s, 1H), 8.40 (s, 1H), 7.52 (d, J=8.2 Hz, 1H), 7.27 (dd, J=11.2, 4.0 Hz, 1H), 7.18 (t, J=7.4 Hz, 1H), 7.16-7.10 (m, 2H), 7.09 (d, J=2.4 Hz, 1H), 6.93 (d, J=8.5 Hz, 1H), 6.85 (dd, J=3.5, 1.8 Hz, 1H), 4.81 (s, 2H), 3.95 (s, 3H), 2.94-2.89 (m, 4H), 2.26 (t, J=6.9 Hz, 2H), 2.14 (s, 6H), 0.94 (t, J=7.1 Hz, 3H).

### N-(2-((2-(dimethylamino)ethyl)(ethyl)amino)-5-((4-(1-methyl-1H-indol-3-yl)-7H-pyrrolo[2,3-d]pyrimidin-2-yl)amino)phenyl)acrylamide (212a)

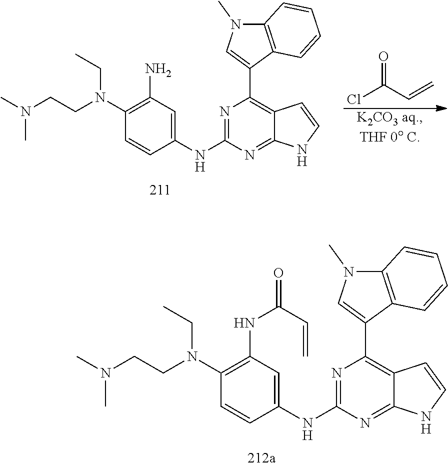

Acryl chloride (0.21 g, 2.33 mmol) was added to the mixture of 211 (0.52 g, 1.11 mmol), K₂CO₃ (0.22 g, 1.59 mmol), water (3 ml) in the tetrahydrofuran (10 ml) at 0° C. The mixture was stirred for 0.5 h. NaOH (0.05 g, 1.25 mmol) was added to the reaction mixture at 0° C. and stirred for 0.5 h. Ethyl acetate (50 ml) was added to the mixture. The organic layer was washed with brine (50 ml*3), and then concentrated under reduced pressure. The residue was purified by flash column chromatography (dichloromethane/methanol=25/1) to give the title product 212a as a white solid (0.184 g, yield 31.73%). LC-MS: m/z 523.4 [M+H]⁺. ¹H NMR (500 MHz, DMSO-d6) δ 11.43 (s, 1H), 10.37 (s, 1H), 9.15 (s, 1H), 8.91 (d, J=8.0 Hz, 1H), 8.54 (d, J=2.6 Hz, 1H), 8.45 (s, 1H), 7.83 (dd, J=8.7, 2.4 Hz, 1H), 7.52 (d, J=8.2 Hz, 1H), 7.33-7.22 (m, 2H), 7.22-7.15 (m, 2H), 6.94-6.87 (m, 1H), 6.40 (dd, J=16.7, 10.2 Hz, 1H), 6.26 (dd, J=17.0, 1.7 Hz, 1H), 5.78 (dd, J=10.2, 1.8 Hz, 1H), 3.95 (s, 3H), 2.98 (q, J=6.7 Hz, 2H), 2.58-2.46 (m, 4H), 2.22 (s, 6H), 0.87 (t, J=7.1 Hz, 3H).

### N-(2-((2-(dimethylamino)ethyl)(ethyl)amino)-5-((4-(1-methyl-1H-indol-3-yl)-7H-pyrrolo[2,3-d]pyrimidin-2-yl)amino)phenyl)propionamide (212b)

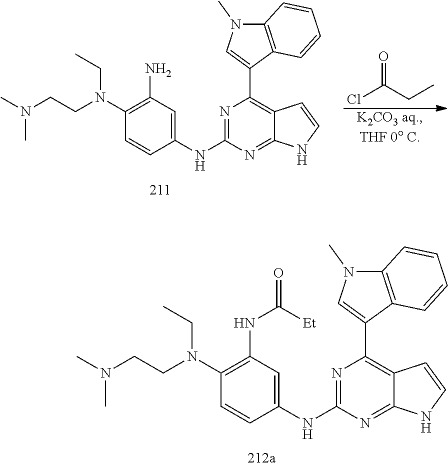

Propionyl chloride (1.0 g, 10.8 mmol) was added dropwise to a mixture of 211 (2.00 g, 4.3 mmol), K₂CO₃ (1.1 g, 8.0 mmol) in tetrahydrofuran (22 ml) and water (15 ml) at 0° C. The reaction mixture was stirred for 1 h. The tetrahydrofuran layer was separated and washed with water twice, then dried over Na₂SO₄ and filtered. The filtrate was concentrated to dryness. The crude was purified by flash column chromatography (eluting with methanol/dichloromethane, from 1/50 to 1/20, v/v) to give the title compound 212b (1.0 g, yield 44.6%) as an off-white solid. LC-MS: m/z 525.8 [M+H]⁺. ¹H NMR (500 MHz, DMSO) δ 11.41 (s, 1H), 10.03 (s, 1H), 9.09 (s, 1H), 8.91 (d, J=8.0 Hz, 1H), 8.44 (d, J=3.7 Hz, 2H), 7.81 (dd, J=8.7, 2.5 Hz, 1H), 7.52 (d, J=8.2 Hz, 1H), 7.30-7.24 (m, 1H), 7.22 (d, J=8.7 Hz, 1H), 7.19-7.14 (m, 2H), 6.88 (dd, J=3.6, 1.8 Hz, 1H), 3.95 (s, 3H), 2.99-2.93 (m, 6.6 Hz, 4H), 2.33 (q, J=7.6 Hz, 2H), 2.20 (s, 6H), 2.20-2.15 (m, 2H), 1.14 (t, J=7.6 Hz, 3H), 0.87 (t, J=7.1 Hz, 3H).

### N-(2-((2-(dimethylamino)ethyl)(ethyl)amino)-5-((4-(1-methyl-1H-indol-3-yl)-7H-pyrrolo[2,3-d]pyrimidin-2-yl)amino)phenyl)acetamide (212c)

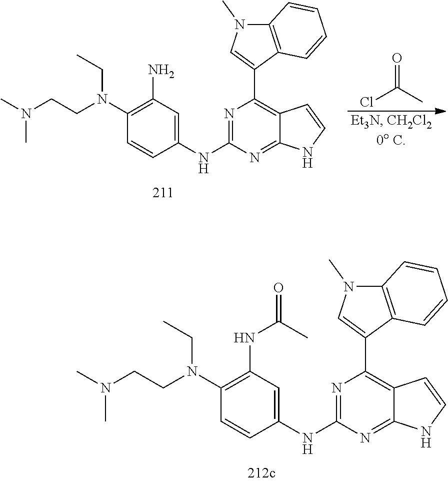

Acetyl chloride (0.05, 0.64 mmol) was dropped to the mixture of 211 (0.21 g, 0.44 mmol) and triethylamine (0.10 g, 0.99 mmol) in dichloromethane at 0° C. And then the mixture was stirred for a further 0.5 h. Then the mixture was concentrated and diluted with ethyl acetate (50 ml). The organic layers were combined, washed with brine (50 ml*3), and then concentrated under reduced pressure. The residue was purified by flash column chromatography (dichloromethane/methanol=20/1) to give the title compound 212c (70 mg, yield 31.19%). LC-MS: m/z 511.5 [M+H]⁺. ¹H NMR (500 MHz, DMSO-d6) δ 11.41 (s, 1H), 10.06 (s, 1H), 9.11 (s, 1H), 8.92 (d, J=8.0 Hz, 1H), 8.43 (d, J=8.6 Hz, 2H), 7.82 (dd, J=8.6, 2.2 Hz, 1H), 7.52 (d, J=8.2 Hz, 1H), 7.27 (t, J=7.2 Hz, 1H), 7.24-7.13 (m, 2H), 6.87 (s, 1H), 3.95 (s, 3H), 2.96 (t, J=7.0 Hz, 4H), 2.51 (s, 2H), 2.26 (s, 6H), 2.08 (s, 3H), 0.88 (t, J=7.0 Hz, 3H).

### N-(2-((2-(dimethylamino)ethyl)(ethyl)amino)-5-((4-(1-methyl-1H-indol-3-yl)-7H-pyrrolo[2,3-d]pyrimidin-2-yl)amino)phenyl)isobutyramide (212d)

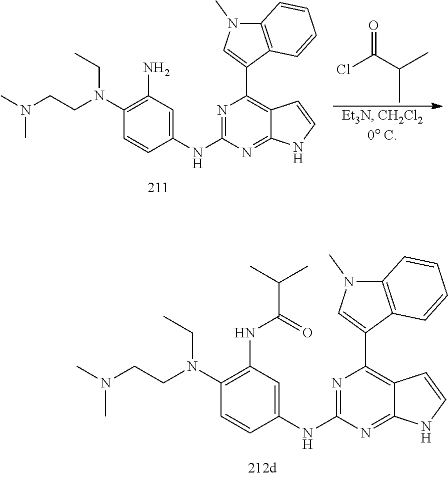

Isobutyryl chloride (0.07 g, 0.66 mmol) was dropped to the mixture of 211 (0.21 g, 0.44 mmol) and triethylamine (0.11 g, 1.08 mol) in dichloromethane at 0° C. And then the mixture was stirred for a further 0.5 h. Then the mixture was concentrated and diluted with ethyl acetate (50 ml). The organic layers were combined, washed with brine (50 ml*3), and then concentrated under reduced pressure. The residue was purified by flash column chromatography (dichloromethane/methanol=20/1) to give the title compound 212d (110 mg, yield 46.44%). LC-MS: m/z 539.8 [M+H]⁺.

### N¹-(2-(dimethylamino)ethyl)-N¹-ethyl-N²-(2-fluoroethyl)-N⁴-(4-(1-methyl-1H-indol-3-yl)-7H-pyrrolo[2,3-d]pyrimidin-2-yl)benzene-1,2,4-triamine (212e)

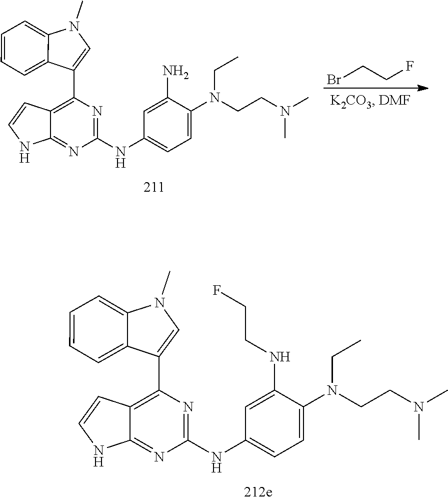

To the mixture of compound 211 (0.22 g, 0.44 mmol), K₂CO₃ (0.13 g, 0.94 mmol) in N,N-dimethylformamide (5 ml), 1-bromo-2-fluoroethan (0.18 g, 1.41 mmol) was added. The reaction mixture was stirred at 45° C. for 4 h. And then the reaction mixture was allowed to cool down, followed by addition of ethyl acetate (50 ml). The organic layer was washed with brine (50 ml*3), and then concentrated under reduced pressure. The residue was purified by preparative thin layer chromatography (dichloromethane/methanol=20/1) to give the compound 212e (74 mg, yield 32.7%). MS-ESI (M+H)⁺:515.9. ¹H NMR (500 MHz, DMSO-d6) δ 10.86 (s, 1H), 8.93 (d, J=7.7 Hz, 2H), 8.46 (s, 1H), 7.52 (d, J=8.2 Hz, 1H), 7.41 (s, 1H), 7.32-7.25 (m, 2H), 7.20 (t, J=7.4 Hz, 1H), 7.07 (d, J=8.0 Hz, 1H), 6.97-6.92 (m, 2H), 5.77 (s, 1H), 4.91 (t, J=4.8 Hz, 1H), 4.82 (t, J=4.8 Hz, 1H), 4.56 (t, J=4.7 Hz, 1H), 4.50 (t, J=4.8 Hz, 1H), 3.95 (s, 3H), 3.20 (t, J=5.5 Hz, 2H), 3.12 (s, 2H), 2.91 (q, J=6.9 Hz, 2H), 2.74 (s, 6H), 0.95 (t, J=7.0 Hz, 3H).

### N¹-(2-(dimethylamino)ethyl)-N¹,N²-diethyl-N⁴-(4-(1-methyl-1H-indol-3-yl)-7H-pyrrolo[2,3-d]pyrimidin-2-yl)benzene-1,2,4-triamine (212f)

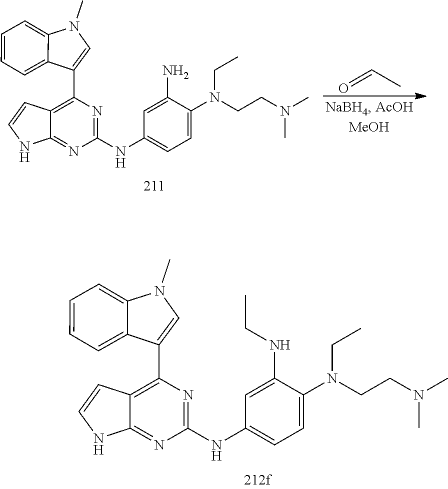

To the mixture of compound 211 (0.14 g, 0.30 mmol), acetic acid (0.2 ml) and acetaldehyde aqueous solution (3 ml) in methanol (6 ml), NaBH₄ (0.30 g, 7.89 mmol) was slowly added at room temperature. The mixture was stirred for 3 h. Ethyl acetate (50 ml) and water (50 ml) was added to the mixture. The organic layer was washed by saturated NaHCO₃ aqueous (50 ml), and then concentrated under reduced pressure. The residue was purified by flash column chromatography (dichloromethane/methanol=20/1) to give the compound 212f (64 mg, yield 42.98%). MS-ESI (M+H)⁺:497.4; ¹H NMR (500 MHz, DMSO-d6) δ 11.38 (s, 1H), 8.93 (d, J=8.0 Hz, 1H), 8.82 (s, 1H), 8.42 (s, 1H), 7.52 (d, J=8.2 Hz, 1H), 7.31-7.23 (m, 2H), 7.22-7.15 (m, 2H), 7.12 (d, J=2.3 Hz, 1H), 6.96 (d, J=8.5 Hz, 1H), 6.87 (dd, J=3.5, 1.8 Hz, 1H), 5.23 (d, J=4.0 Hz, 1H), 3.95 (s, 3H), 3.14 (q, J=7.1 Hz, 2H), 3.01 (q, J=7.2 Hz, 6H), 2.89 (q, J=6.9 Hz, 2H), 2.53-2.49 (m, 4H), 0.93 (t, J=7.0 Hz, 3H).

### Procedure for the Synthesis of 216

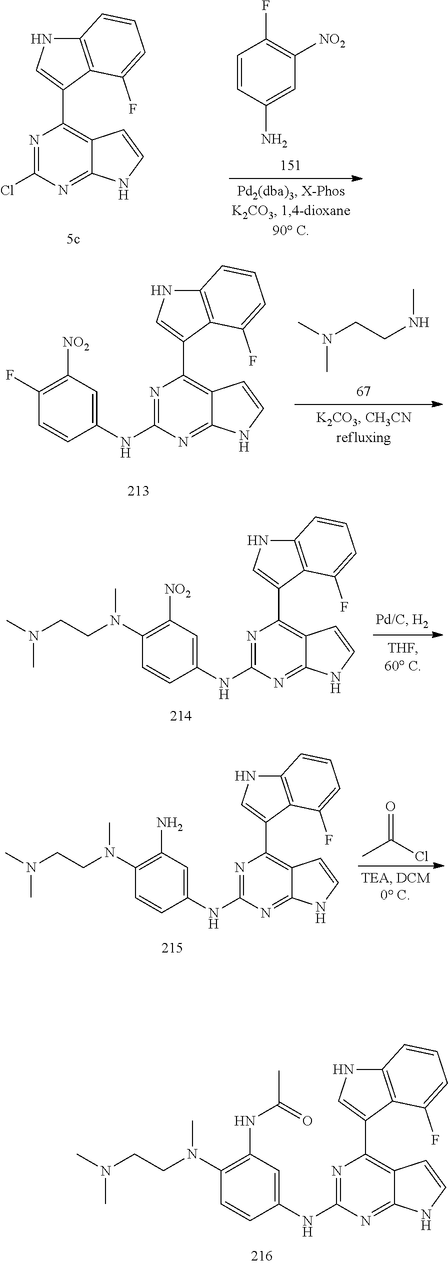

### 4-(4-fluoro-1H-indol-3-yl)-N-(4-fluoro-3-nitrophenyl)-7H-pyrrolo[2,3-d]pyrimidin-2-amine (213)

X-Phos (0.1 g, 0.21 mmol), Pd₂(dba)₃ (0.1 g, 0.11 mmol), K₂CO₃ (0.87 g, 6.29 mmol) and the compound 5c (0.9 g, 3.14 mmol) were added to a solution of 4-fluoro-3-nitroaniline 151 (0.5 g, 3.20 mmol) in 1,4-dioxane (50 ml). The resulting mixture was stirred at 90° C. for 4 h. After cooling to room temperature, the reaction mixture was filtered and concentrated. The residue was dissolved in ethyl acetate (50 ml) and washed with brine (50 ml*3). The organic layer was separated, and then concentrated under reduced pressure to give the crude product 213, which was used in the next step without further purification. LC-MS: m/z 407.6 [M+H]⁺.

### N¹-(2-(dimethylamino)ethyl)-N⁴-(4-(4-fluoro-1H-indol-3-yl)-7H-pyrrolo[2,3-d]pyrimidin-2-yl)-N¹-methyl-2-nitrobenzene-1,4-diamine (214)

N¹,N¹,N-Trimethylethane-1,2-diamine 67 (0.6 g, 6.81 mmol) was added to a mixture of above compound 213 and K₂CO₃ (0.8 g, 5.78 mmol) in acetonitrile (30 mL). The resulting mixture was heated at refluxing temperature for 4 h. Then the reaction mixture was allowed to cool down and quench with water (100 ml). The mixture was extracted with ethyl acetate (50 ml*3). The organic layers were combined, washed with brine (100 ml*3), and then concentrated under reduced pressure to give the title compound 214, which was used in the next step without further purification. LC-MS: m/z 489.6 [M+H]⁺.

### N¹-(2-(dimethylamino)ethyl)-N₄-(4-(4-fluoro-1H-indol-3-yl)-7H-pyrrolo[2,3-d]pyrimidin-2-yl)-N1-methylbenzene-1,2,4-triamine (215)

The above compound 214 was mixed with 5% Pd/C (50 mg, 0.02 mmol) in THF (50 ml). The resulting mixture was heated at 60° C. overnight under hydrogen atmosphere. The reaction mixture was filtered and concentrated. The residue was purified by flash column chromatography (dichloromethane/methanol=10/1) to give the compound 215 (0.2 g, yield 14.0% over three steps). LC-MS: m/z 459.1 [M+H]⁺.

### N-(2-((2-(dimethylamino)ethyl)(methyl)amino)-5-((4-(4-fluoro-1H-indol-3-yl)-7H-pyrrolo[2,3-d]pyrimidin-2-yl)amino)phenyl)acetamide (216)

Acetyl chloride (37.7 mg, 0.48 mmol) was dropped into a solution of compound 215 (0.2 g, 0.43 mmol) and triethylamine (0.10 g, 0.99 mmol) in dichloromethane (20 ml) at 0° C. The reaction mixture was stirred for 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 216 (70 mg, yield 32.55%). LC-MS: m/z 501.9 [M+H]⁺.

### Procedure for the Synthesis of 218

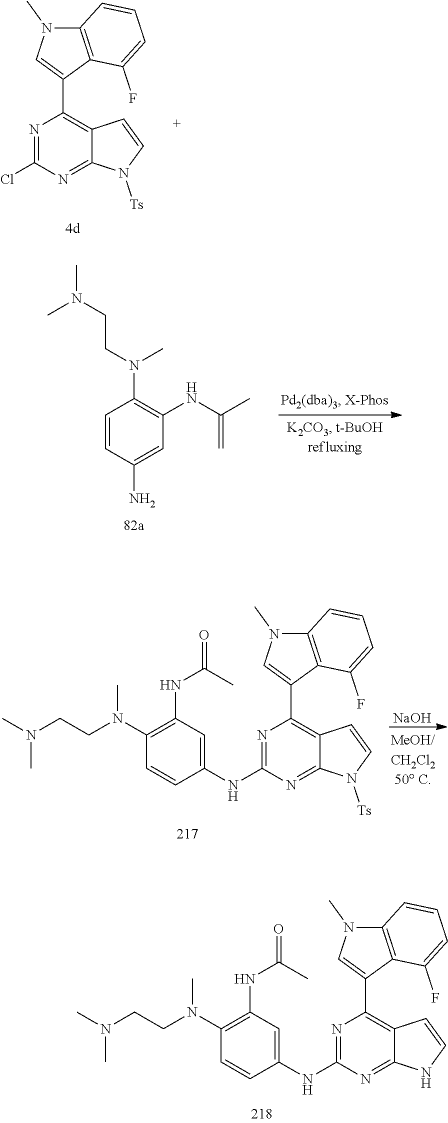

### N-(2-((2-(dimethylamino)ethyl)(methyl)amino)-5-((4-(4-fluoro-1-methyl-1H-indol-3-yl)-7-tosyl-7H-pyrrolo[2,3-d]pyrimidin-2-yl)amino)phenyl)acetamide (217)

A mixture of intermediate 4d (350 mg, 0.77 mmol), intermediate 82a (137.5 mg, 0.55 mmol), Pd₂(dba)₃ (56.1 mg, 0.06 mmol), X-Phos (66.7 mg, 0.14 mmol) and K₂CO₃ (227.6 mg, 1.65 mmol) in t-BuOH (20 mL) was stirred under N₂ at refluxing temperature for 19 h. After cooling to room temperature, the reaction mixtures was filtered through Celite®. The filter cake was washed with methanol/dichloromethane and the filtrates were concentrated under reduced pressure to dryness. The crude was purified by flash column chromatography (dichloromethane/methanol=20/1) to give the title compound 217 (200 mg, yield 54.5%) as a brown solid.

### N-(2-((2-(dimethylamino)ethyl)(methyl)amino)-5-((4-(4-fluoro-1-methyl-1H-indol-3-yl)-7H-pyrrolo[2,3-d]pyrimidin-2-yl)amino)phenyl)acetamide (218)

NaOH (51.0 mg, 5.26 mmol) was added into a solution of compound 217 (200 mg, 0.30 mmol) in methanol (5 mL) and dichloromethane (5 mL). The reaction mixture was stirred at 50° C. for 2.5 h. Solvents were removed in vacuo. The crude was purified by flash column chromatography (dichloromethane/methanol=20/1) to give the title compound 218 (15 mg, yield 9.7%) as a light-yellow solid.

### Procedure for the Synthesis of 222

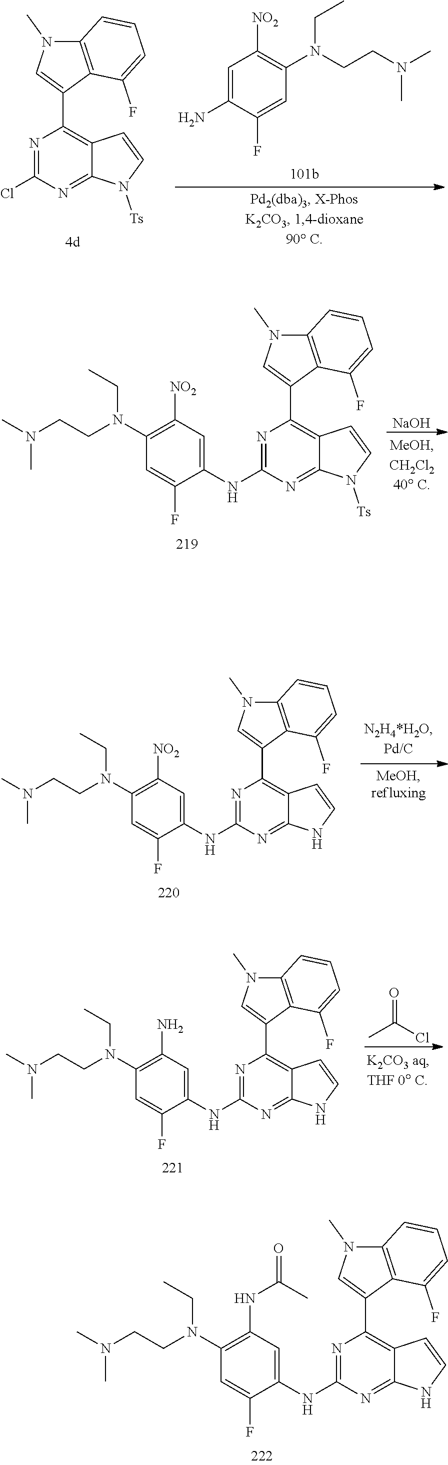

### N¹-(2-(dimethylamino)ethyl)-N¹-ethyl-5-fluoro-N⁴-(4-(4-fluoro-1-methyl-1H-indol-3-yl)-7-tosyl-7H-pyrrolo[2,3-d]pyrimidin-2-yl)-2-nitrobenzene-1,4-diamine (219)

A mixture of intermediate 4d (507 mg, 1.11 mmol), intermediate 101b (308 mg, 1.14 mmol), Pd₂(dba)₃ (113 mg, 0.12 mmol), X-Phos (110 mg, 0.23 mmol) and K₂CO₃ (476 mg, 3.44 mmol) in t-BuOH (30 mL) was stirred under N₂ at 90° C. for 17 h. After cooling to room temperature, the reaction mixture was filtered through Celite® and the filtrates were concentrated under reduced pressure to dryness. The crude was purified by flash column chromatography (dichloromethane/methanol=10/1) to give the title compound 219 (362 mg, yield 47.2%) as a brown oil.

### N¹-(2-(dimethylamino)ethyl)-N¹-ethyl-5-fluoro-N⁴-(4-(4-fluoro-1-methyl-1H-indol-3-yl)-7H-pyrrolo[2,3-d]pyrimidin-2-yl)-2-nitrobenzene-1,4-diamine (220)

NaOH (82 mg, 2.05 mmol) was added into a solution of compound 219 (221 mg, 0.32 mmol) in methanol (10 mL) and dichloromethane (1 mL). The reaction mixture was stirred at 40° C. for 4 h. Solvents were removed in vacuo followed by addition of dichloromethane (20 mL) and H₂O (10 mL). The water phase was neutralized to pH 7 with acetic acid, and then extracted with dichloromethane. The organic layers were separated, dried over anhydrous Na₂SO₄ and then concentrated to give the title compound 220 (132 mg) as a red solid, which was used in the next step without further purification.

### N¹-(2-(dimethylamino)ethyl)-N¹-ethyl-5-fluoro-N⁴-(4-(4-fluoro-1-methyl-1H-indol-3-yl)-7H-pyrrolo[2,3-d]pyrimidin-2-yl)benzene-1,2,4-triamine (221)

The mixture of compound 220 (132 mg, 0.25 mmol), Pd/C (5%, 75 mg, 0.04 mmol) and hydrazine hydrate (80%, 5 ml, 80 mmol) in methanol (20 ml) was stirred at refluxing temperature for 3.5 h. The mixture was filtered, concentrated and then extracted with ethyl acetate (50 ml*3). The organic layers were combined, washed with brine (100 ml*3), and then concentrated under reduced pressure to give the compound 221 (93 mg) as a yellow oil, which was used in the next step without further purification.

### N-(2-((2-(dimethylamino)ethyl)(ethyl)amino)-4-fluoro-5-((4-(4-fluoro-1-methyl-1H-indol-3-yl)-7H-pyrrolo[2,3-d]pyrimidin-2-yl)amino)phenyl)acetamide (222)

To a solution of 221 (93 mg, 0.18 mmol) in tetrahydrofuran (5 mL) and saturated K₂CO₃ aqueous (1 mL) in was added acetyl chloride (120 mg, 1.52 mmol) dropwise at 0° C. The reaction was stirred at room temperature for 3 h, and then quenched with H₂O. The mixture was extracted with ethyl acetate (10 mL). The organic layer was washed with brine, dried over Na₂SO₄ and concentrated. The residue was purified by flash column chromatography (dichloromethane/methanol=20/1) to afford the title compound 222 (52 mg, 31.3% yield over three steps) as a slight yellow solid.

### Procedures for the Syntheses of 227a-d

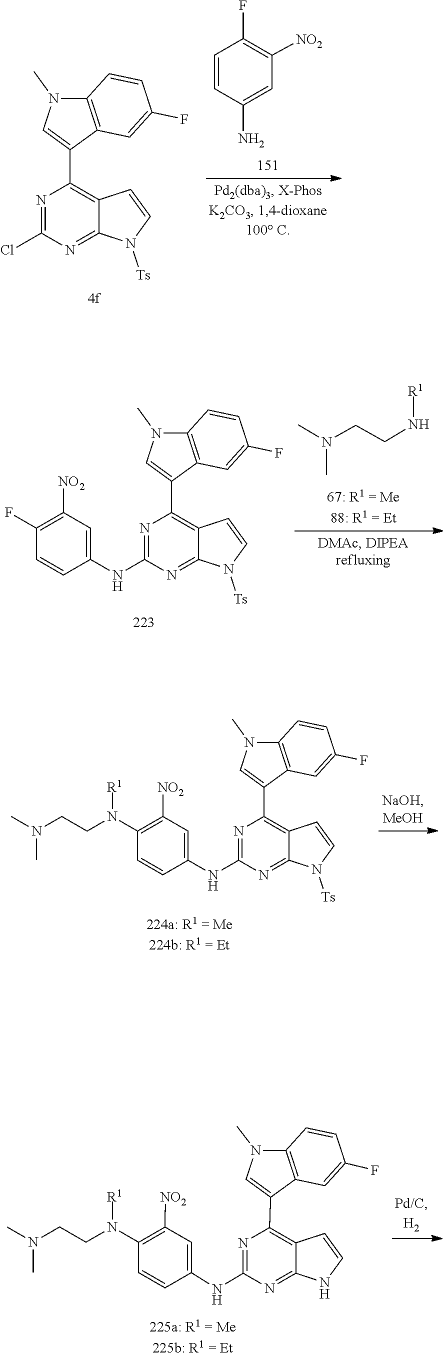

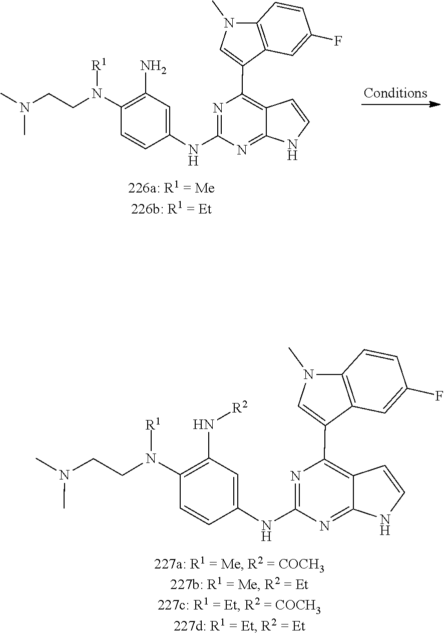

### 4-(5-fluoro-1-methyl-1H-indol-3-yl)-N-(4-fluoro-3-nitrophenyl)-7-tosyl-7H-pyrrolo[2,3-d]pyrimidin-2-amine (223)

X-Phos (0.2 g, 0.42 mmol), Pd₂(dba)₃ (0.2 g, 0.21 mmol), K2CO₃ (1.21 g, 8.70 mmol) and the intermediate 4f (2.0 g, 4.41 mmol) were added to a solution of 4-fluoro-3-nitroaniline 151 (0.68 g, 4.35 mmol) in 1,4-dioxane (40 ml). The resulting mixture was stirred at 100° C. for 4 h. After cooling down, the reaction mixture was filtered and concentrated. The residue was stirred in ethyl acetate (30 ml) for 0.5 h, and then filtered. The filter cake was dried in vacuo to give the title compound 223 (2.2 g, yield 87.9%).

### N⁴-(4-(5-fluoro-1-methyl-1H-indol-3-yl)-7-tosyl-7H-pyrrolo[2,3-d]pyrimidin-2-yl)-N¹-(2-(dimethylamino)ethyl)-N¹-methyl-2-nitrobenzene-1,4-diamine (224a)

N¹,N¹,N-Trimethylethane-1,2-diamine 67 (0.4 g, 4.54 mmol) was added to a mixture of compound 223 (1.18 g, 2.05 mmol) and N, N-diisopropylethylamine (0.4 g, 3.10 mmol) in N,N-dimethyl acetamide (20 mL). The resulting mixture was heated at refluxing temperature for 3.5 h. Then the reaction mixture was allowed to cool down and quench with water (100 ml). The mixture was extracted with ethyl acetate (50 ml*3). The organic layers were combined, washed with brine (100 ml*3), and then concentrated under reduced pressure to give the title compound 224a, which was used for next step without further purification. LC-MS: m/z 657.4 [M+H]⁺.

### N¹-(2-(dimethylamino)ethyl)-N¹-ethyl-N⁴-(4-(5-fluoro-1-methyl-1H-indol-3-yl)-7-tosyl-7H-pyrrolo[2,3-d]pyrimidin-2-yl)-2-nitrobenzene-1,4-diamine (224b)

N¹-ethyl-N²,N²-dimethylethane-1,2-diamine 88 (0.40 g, 3.4 mmol) was added to a mixture of compound 223 (0.94 g, 1.6 mmol) and N, N-diisopropylethylamine (0.5 g, 3.8 mmol) in N,N-dimethyl acetamide (20 mL). The resulting mixture was heated at refluxing temperature for 6 h. Then the reaction mixture was allowed to cool down and quench with water (100 ml). The mixture was extracted with ethyl acetate (50 ml*3). The organic layers were combined, washed with brine (100 ml*3), and then concentrated under reduced pressure to give the title compound 224b, which was used for next step without further purification. LC-MS: m/z 671.2 [M+H]⁺.

### N¹-(2-(dimethylamino)ethyl)-N⁴-(4-(5-fluoro-1-methyl-1H-indol-3-yl)-7H-pyrrolo[2,3-d]pyrimidin-2-yl)-N¹-methyl-2-nitrobenzene-1,4-diamine (225a)

A mixture of the above compound 224a and NaOH (3.0 g, 75 mmol) in methanol (30 ml) was stirred at 40° C. for 0.5 h. Then the reaction mixture was allowed to cool down and quench with water (100 ml). The mixture was extracted with dichloromethane (50 ml*3). The organic layers were combined, washed with brine (100 ml*3), and then concentrated under reduced pressure to give the title compound 225a, which was used for next step without further purification. LC-MS: m/z 503.4 [M+H]⁺.

### N¹-(2-(dimethylamino)ethyl)-N¹-ethyl-N⁴-(4-(5-fluoro-1-methyl-1H-indol-3-yl)-7H-pyrrolo[2,3-d]pyrimidin-2-yl)-2-nitrobenzene-1,4-diamine (225b)

A mixture of the above compound 224b and NaOH (2.0 g, 50 mmol) in methanol (30 ml) was stirred at 40° C. for 0.5 h. Then the reaction mixture was allowed to cool down and quench with water (100 ml). The mixture was extracted with dichloromethane (50 ml*3). The organic layers were combined, washed with brine (100 ml*3), and then concentrated under reduced pressure to give the title compound 225b, which was used for next step without further purification. LC-MS: m/z 517.2 [M+H]⁺.

### N¹-(2-(dimethylamino)ethyl)-N⁴-(4-(5-fluoro-1-methyl-1H-indol-3-yl)-7H-pyrrolo[2,3-d]pyrimidin-2-yl)-N¹-methylbenzene-1,2,4-triamine (226a)

The above compound 225a was mixed with Pd/C (50 mg, 0.24 mmol) in THF (50 ml). The resulting mixture was heated at 60° C. overnight under hydrogen atmosphere. The reaction mixture was filtered and concentrated. The residue was purified by flash column chromatography (dichloromethane/methanol=10/1) to give the compound 226a (0.47 g, yield 48.5% over three steps). LC-MS: m/z 473.7 [M+H]⁺.

### N¹-(2-(dimethylamino)ethyl)-N¹-ethyl-N⁴-(4-(5-fluoro-1-methyl-1H-indol-3-yl)-7H-pyrrolo[2,3-d]pyrimidin-2-yl)benzene-1,2,4-triamine (226b)

The above compound 225b was mixed with Pd/C (0.1 g, 0.48 mmol) in THF (20 ml). The resulting mixture was stirred at room temperature overnight under hydrogen atmosphere. The reaction mixture was filtered and concentrated. The residue was purified by flash column chromatography (dichloromethane/methanol=10/1) to give the compound 226b (0.3 g, yield 38.6% over three steps). LC-MS: m/z 487.3 [M+H]⁺.

### N-(2-((2-(dimethylamino)ethyl)(methyl)amino)-5-((4-(5-fluoro-1-methyl-1H-indol-3-yl)-7H-pyrrolo[2,3-d]pyrimidin-2-yl)amino)phenyl)acetamide (227a)

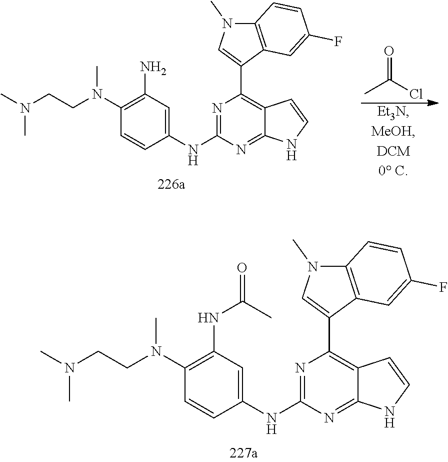

The compound 226a (0.3 g, 0.63 mmol), triethylamine (0.15 g, 1.48 mmol), methanol (10 ml) and dichloromethane (10 ml) were added to the reaction flask. The resulting mixture was stirred at 0° C. Acetyl chloride (0.07 g, 0.73 mmol) was dropped into the reaction. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 227a (108 mg, yield 33.3%). LC-MS: m/z 515.26 [M+H]⁺. ¹H NMR (500 MHz, DMSO-d6) δ 11.40 (s, 1H), 10.01 (s, 1H), 9.14 (s, 1H), 8.69 (d, J=13.2 Hz, 1H), 8.51 (s, 1H), 8.41 (s, 1H), 7.76 (d, J=2.4 Hz, 1H), 7.52 (dd, J=8.9, 4.6 Hz, 1H), 7.22 (d, J=8.7 Hz, 1H), 7.19-7.14 (m, 1H), 7.10 (td, J=9.1, 2.6 Hz, 1H), 6.88 (dd, J=3.5, 1.8 Hz, 1H), 3.95 (s, 3H), 2.82 (t, J=5.5 Hz, 2H), 2.67 (s, 3H), 2.28 (t, J=5.6 Hz, 2H), 2.23 (s, 6H), 2.07 (s, 3H).

### N¹-(2-(dimethylamino)ethyl)-N²-ethyl-N⁴-(4-(5-fluoro-1-methyl-1H-indol-3-yl)-7H-pyrrolo[2,3-d]pyrimidin-2-yl)-N¹-methylbenzene-1,2,4-triamine (227b)

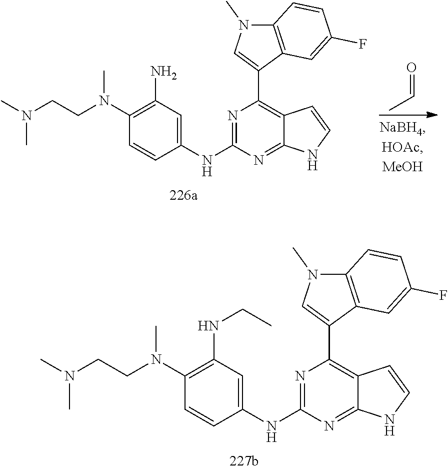

The compound 226a (170 mg, 0.35 mmol), acetic acid (0.1 mL), acetaldehyde aqueous solution (5 ml) and methanol (10 ml) were added to the reaction flask (50 ml). The resulting mixture was cooled by ice-salt bath. Then NaBH₄ (0.15 g, 3.91 mmol) was added to the reaction mixture. The resulting mixture was stirred for 1 h. When TLC (thin layer chromatography dichloromethane/methanol=10/1) indicated that the reaction was completed, the reaction mixture was concentrated. The residue was dissolved in ethyl acetate (50 ml). The organic layer was washed with water (50 ml*3), and then concentrated under reduced pressure. The residue was purified by flash column chromatography (dichloromethane/methanol=20/1) to give the compound 227b (30 mg, yield 17.2%). LC-MS: m/z 501.25 [M+H]⁺. ¹H NMR (500 MHz, DMSO-d6) δ 11.40 (s, 1H), 10.01 (s, 1H), 9.14 (s, 1H), 8.69 (d, J=13.2 Hz, 1H), 8.51 (s, 1H), 8.41 (s, 1H), 7.76 (d, J=11.0 Hz, 1H), 7.52 (dd, J=8.9, 4.6 Hz, 1H), 7.22 (d, J=8.7 Hz, 1H), 7.18-7.15 (m, 1H), 7.10 (td, J=9.1, 2.6 Hz, 1H), 6.88 (dd, J=3.5, 1.8 Hz, 1H), 3.95 (s, 3H), 2.82 (t, J=5.5 Hz, 2H), 2.67 (s, 3H), 2.53-2.49 (m, 2H), 2.28 (t, J=5.6 Hz, 2H), 2.23 (s, 6H), 2.07 (s, 3H).

### N-(2-((2-(dimethylamino)ethyl)(ethyl)amino)-5-((4-(5-fluoro-1-methyl-1H-indol-3-yl)-7H-pyrrolo[2,3-d]pyrimidin-2-yl)amino)phenyl)acetamide (227c)

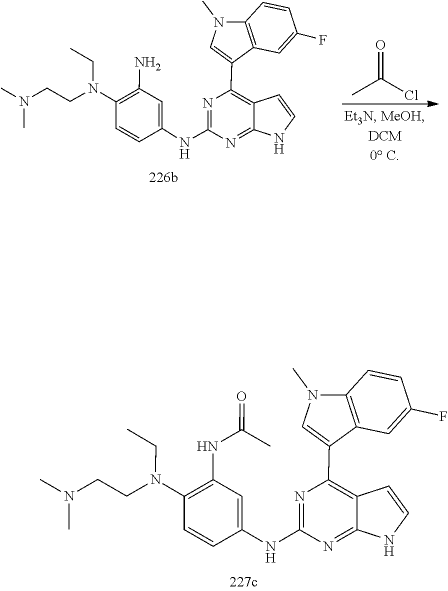

The compound 226b (0.3 g, 0.61 mmol), triethylamine (0.15 g, 1.48 mmol), methanol (20 ml) and dichloromethane (15 ml) were added to the reaction flask. The resulting mixture was stirred at 0° C. Acetyl chloride (0.07 g, 0.73 mmol) was dropped into the reaction mixture. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 227c (28 mg yield 8.7%). LC-MS: m/z 529.5 [M+H]⁺.

### N¹-(2-(dimethylamino)ethyl)-N¹,N²-diethyl-N⁴-(4-(5-fluoro-1-methyl-1H-indol-3-yl)-7H-pyrrolo[2,3-d]pyrimidin-2-yl)benzene-1,2,4-triamine (227d)

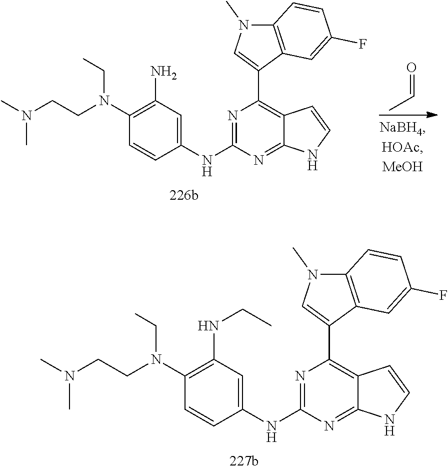

The compound 226b (150 mg, 0.30 mmol), acetic acid (0.1 ml), acetaldehyde aqueous solution (5 ml) and methanol (10 ml) were added to the reaction flask. The resulting mixture was cooled by ice-salt bath. Then NaBH₄ (0.15 g, 3.96 mmol) was added to the reaction mixture. The resulting mixture was stirred for 1 h. When TLC (thin layer chromatography dichloromethane/methanol=10/1) indicated that the reaction was completed, the reaction mixture was concentrated. The residue was dissolved in ethyl acetate (50 ml) and washed with water (50 ml*3). The organic layer was concentrated under reduced pressure. The residue was purified by flash column chromatography (dichloromethane/methanol=20/1) to give the compound 227d (8 mg, yield 5.2%). LC-MS: m/z 515.8 [M+H]⁺.

### General Procedures for the Syntheses of 232a and 232b

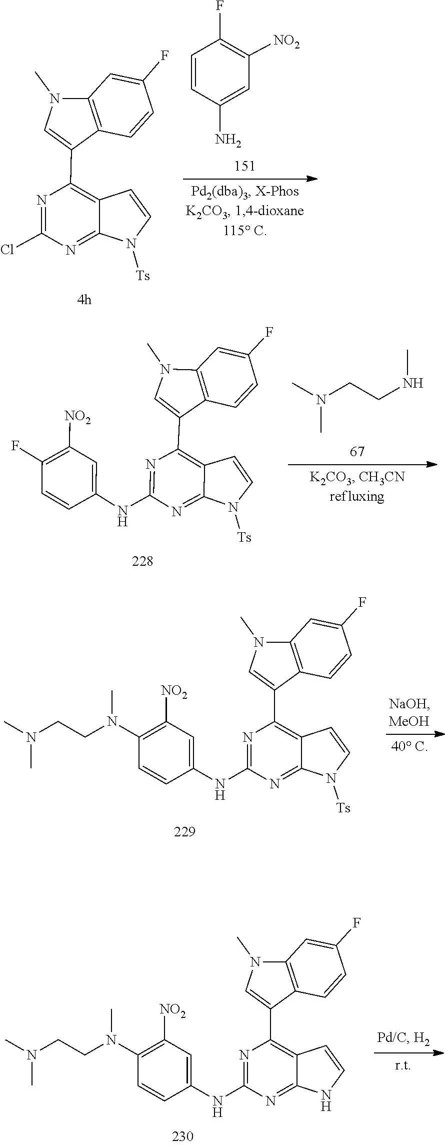

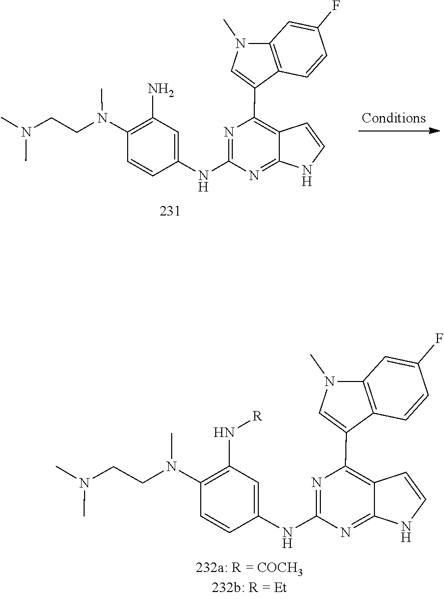

### 4-(6-fluoro-1-methyl-1H-indol-3-yl)-N-(4-fluoro-3-nitrophenyl)-7-tosyl-7H-pyrrolo[2,3-d]pyrimidin-2-amine (228)

X-Phos (0.2 g, 0.42 mmol), Pd₂(dba)₃ (0.2 g, 0.21 mmol), K₂CO₃ (1.21 g, 8.75 mmol) and intermediate 4h (2.0 g, 4.40 mmol) were added to a solution of 4-fluoro-3-nitroaniline 151 (0.68 g, 4.36 mmol) in 1,4-dioxane (40 ml). The resulting mixture was stirred at 115° C. for 4 h. After cooling down, water (100 ml) was added into the reaction mixture. The suspension was filtered and the filter cake was dried in vacuo to give the title compound 228 (2.5 g, yield 98.8%).

### N¹-(2-(dimethylamino)ethyl)-N⁴-(4-(6-fluoro-1-methyl-1H-indol-3-yl)-7-tosyl-7H-pyrrolo[2,3-d]pyrimidin-2-yl)-N¹-methyl-2-nitrobenzene-1,4-diamine (229)

N¹,N¹,N-Trimethylethane-1,2-diamine 67 (0.58 g, 6.59 mmol) was added to a mixture of compound 228 (2.5 g, 4.35 mmol) and K₂CO₃ (1.2 g, 8.68 mmol) in acetonitrile (60 mL). The resulting mixture was heated at refluxing temperature for 2 h. Then the reaction mixture was allowed to cool down followed by filtration and concentration. The residue was dissolved in dichloromethane (100 ml). The organic layers were washed with brine (100 ml*3), and then concentrated under reduced pressure to give the title compound 229 (2.3 g, yield 80.5%), which was used in the next step without further purification. LC-MS: m/z 656.9 [M+H]⁺.

### N¹-(2-(dimethylamino)ethyl)-N⁴-(4-(6-fluoro-1-methyl-1H-indol-3-yl)-7H-pyrrolo[2,3-d]pyrimidin-2-yl)-N¹-methyl-2-nitrobenzene-1,4-diamine (230)

A mixture of the above compound 229 (2.3 g, 3.50 mmol) and NaOH (4.0 g, 100 mmol) in methanol (30 ml) was stirred at 40° C. for 0.5 h. Then the reaction mixture was allowed to cool down and quench with water (100 ml). The mixture was extracted with dichloromethane (50 ml*3). The organic layers were combined, washed with brine (100 ml*3), and then concentrated under reduced pressure to give the title compound 230 (2.1 g), which was used in the next step without further purification. LC-MS: m/z 503.2 [M+H]⁺.

### N¹-(2-(dimethylamino)ethyl)-N⁴-(4-(6-fluoro-1-methyl-1H-indol-3-yl)-7H-pyrrolo[2,3-d]pyrimidin-2-yl)-N¹-methylbenzene-1,2,4-triamine (231)

The above compound 230 was mixed with 5% Pd/C (0.2 g, 0.09 mmol) in methanol (50 ml). The resulting mixture was stirred at room temperature overnight under hydrogen atmosphere. The reaction mixture was filtered and concentrated. The residue was purified by flash column chromatography (dichloromethane/methanol=10/1) to give the compound 231 (0.6 g, yield 36.3%). LC-MS: m/z 473.7 [M+H]⁺.

### N-(2-((2-(dimethylamino)ethyl)(methyl)amino)-5-((4-(6-fluoro-1-methyl-1H-indol-3-yl)-7H-pyrrolo[2,3-d]pyrimidin-2-yl)amino)phenyl)acetamide (232a)

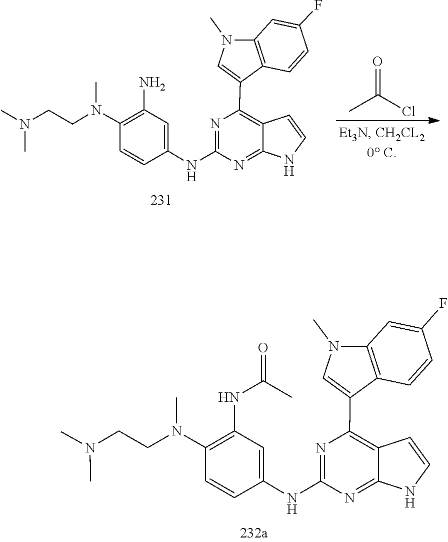

The compound 231 (0.3 g, 0.63 mmol), triethylamine (0.12 g, 1.18 mmol) and dichloromethane (20 ml) were added to the reaction flask. The resulting mixture was stirred at 0° C. Acetyl chloride (0.07 g, 0.90 mmol) was dropped into the reaction mixture. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 232a (140 mg, yield 43.2%). LC-MS: m/z 515.6 [M+H]⁺. ¹H NMR (500 MHz, DMSO-d6) δ 11.42 (s, 1H), 9.88 (s, 1H), 9.10 (s, 1H), 8.94 (dd, J=8.8, 5.9 Hz, 1H), 8.44 (s, 1H), 8.38 (s, 1H), 7.77 (dd, J=8.7, 2.4 Hz, 1H), 7.40 (dd, J=10.1, 2.4 Hz, 1H), 7.20 (d, J=8.7 Hz, 1H), 7.16 (d, J=2.3 Hz, 1H), 7.00 (td, J=9.4, 2.4 Hz, 1H), 6.86 (dd, J=3.6, 1.8 Hz, 1H), 3.91 (s, 3H), 2.64 (s, 3H), 2.52-2.49 (m, 5H), 2.37 (s, 5H), 2.12 (s, 3H).

### N¹-(2-(dimethylamino)ethyl)-N²-ethyl-N⁴-(4-(6-fluoro-1-methyl-1H-indol-3-yl)-7H-pyrrolo[2,3-d]pyrimidin-2-yl)-N¹-methylbenzene-1,2,4-triamine (232b)

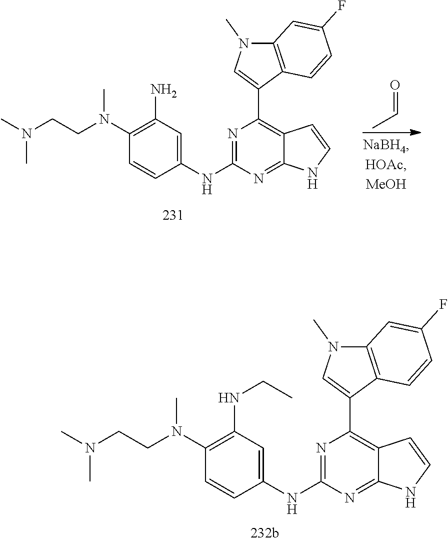

The compound 231 (0.3 g, 0.63 mmol), acetic acid (0.1 ml), acetaldehyde aqueous solution (5 ml) and methanol (10 ml) were added to the reaction flask. The resulting mixture was cooled by ice-salt bath. Then NaBH₄ (0.15 g, 3.94 mmol) was added to the reaction mixture. The resulting mixture was stirred for 1 h. When TLC (thin layer chromatography dichloromethane/methanol=10/1) indicated that the reaction was completed, the reaction mixture was concentrated. The residue was dissolved in ethyl acetate (50 ml) and washed with water (50 ml*3). The organic layer was concentrated under reduced pressure. The residue was purified by flash column chromatography (dichloromethane/methanol=20/1) to give the compound 232b (120 mg, yield 38.1%). LC-MS: m/z 501.8 [M+H]⁺.

### Procedure for the Synthesis of 237

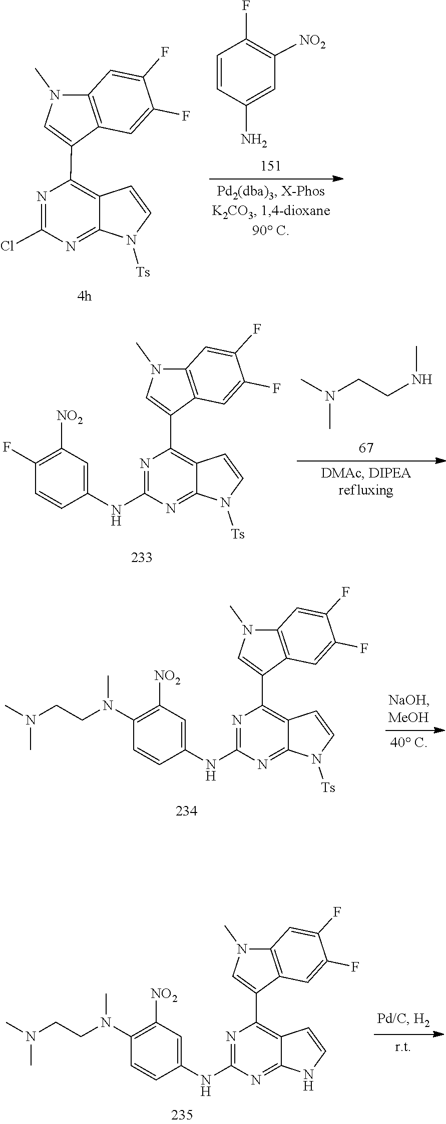

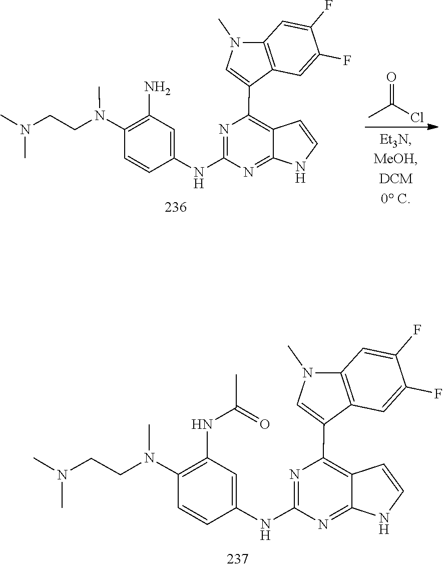

### 4-(5,6-difluoro-1-methyl-1H-indol-3-yl)-N-(4-fluoro-3-nitrophenyl)-7-tosyl-7H-pyrrolo[2,3-d]pyrimidin-2-amine (233)

X-Phos (0.2 g, 0.42 mmol), Pd₂(dba)₃ (0.2 g, 0.21 mmol), K₂CO₃ (1.21 g, 8.75 mmol) and the intermediate 41 (2.0 g, 4.23 mmol) were added to a solution of 4-fluoro-3-nitroaniline 151 (0.61 g, 3.91 mmol) in 1,4-dioxane (50 ml). The resulting mixture was stirred at 90° C. for 4 h. After cooling down, the reaction mixture was filtered. Water (100 ml) was added to the filtrate and stirred for 0.5 h. Then the mixture was filtered and the filter cake was dried in vacuo to give the title compound 233 (2.6 g), which was used for next step without further purification.

### N⁴-(4-(5,6-difluoro-1-methyl-1H-indol-3-yl)-7-tosyl-7H-pyrrolo[2,3-d]pyrimidin-2-yl)-N¹-(2-(dimethylamino)ethyl)-N¹-methyl-2-nitrobenzene-1,4-diamine (234)

The above compound 233 (1.5 g, 2.5 mmol), intermediate 67 (0.6 g, 5.8 mmol), N,N-diisopropylethylamin (0.7 g, 5.4 mmol) and dimethylacetamide (20 ml) were added to the reaction flask. The resulting mixture was heated at refluxing temperature for 6 h. Then the reaction mixture was allowed to cool down and quench with water (100 ml). The mixture was extracted with ethyl acetate (50 ml*3). The organic layers were combined, washed with brine (100 ml*3), and then concentrated under reduced pressure to give the title compound 234 (2.1 g), which was used for next step without further purification. LC-MS: m/z 675.3 [M+H]⁺.

### N⁴-(4-(5,6-difluoro-1-methyl-1H-indol-3-yl)-7H-pyrrolo[2,3-d]pyrimidin-2-yl)-N¹-(2-(dimethylamino)ethyl)-N¹-methyl-2-nitrobenzene-1,4-diamine (235)

A mixture of the above compound 234 and NaOH (3.1 g, 77.5 mmol) in methanol (30 ml) was stirred at 40° C. for 0.5 h. Then the reaction mixture was allowed to cool down and quench with water (100 ml). The mixture was extracted with dichloromethane (50 ml*3). The organic layers were combined, washed with brine (100 ml*3), and then concentrated under reduced pressure. The residue was purified by flash column chromatography (dichloromethane/methanol=20/1) to give the compound 235 (300 mg, yield 25.5% over three steps). LC-MS: m/z 521.5 [M+H]⁺.

### N⁴-(4-(5,6-difluoro-1-methyl-1H-indol-3-yl)-7H-pyrrolo[2,3-d]pyrimidin-2-yl)-N¹-(2-(dimethylamino)ethyl)-N¹-methylbenzene-1,2,4-triamine (236)

The compound 235 (0.3 g, 0.57 mmol) was mixed with 5% Pd/C (20 mg, 0.01 mmol) in methanol (20 ml). The resulting mixture was stirred at room temperature overnight under hydrogen atmosphere. The reaction mixture was filtered and concentrated. The residue was purified by flash column chromatography (dichloromethane/methanol=10/1) to give the compound 236 (0.2 g, yield 71.6%). LC-MS: m/z 491.5 [M+H]⁺.

### N-(5-((4-(5,6-difluoro-1-methyl-1H-indol-3-yl)-7H-pyrrolo[2,3-d]pyrimidin-2-yl)amino)-2-((2-(dimethylamino)ethyl)(methyl)amino)phenyl)acetamide (237)

The compound 236 (0.2 g, 0.41 mmol), triethylamine (0.10 g, 0.99 mmol), methanol (20 ml) and dichloromethane (15 ml) were added to the reaction flask. The resulting mixture was stirred at 0° C. Acetyl chloride (0.07 g, 0.73 mmol) was dropped into the reaction mixture. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 237 (8 mg, yield 3.67%). LC-MS: m/z 533.2 [M+H]⁺.

### Procedure for the Synthesis of 239

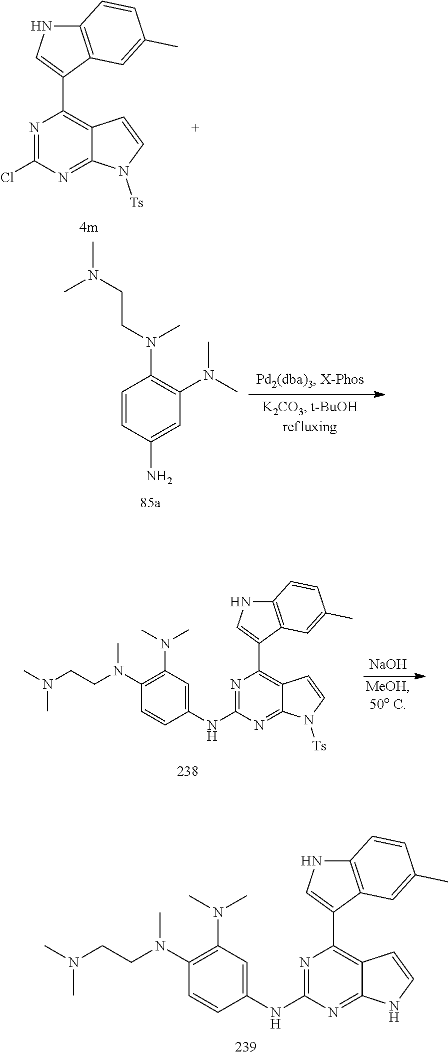

### N¹-(2-(dimethylamino)ethyl)-N¹,N²,N²-trimethyl-N⁴-(4-(5-methyl-1H-indol-3-yl)-7-tosyl-7H-pyrrolo[2,3-d]pyrimidin-2-yl)benzene-1,2,4-triamine (238)

A mixture of intermediate 4m (393 mg, 0.90 mmol), intermediate 85a (150 mg, 0.63 mmol), Pd₂(dba)₃ (85 mg, 0.09 mmol), X-Phos (91 mg, 0.19 mmol) and K₂CO₃ (371 mg, 2.68 mmol) in t-BuOH (20 mL) was stirred under N₂ at refluxing temperature for 18 h. After cooling to room temperature, the reaction mixtures was filtered through Celite® and the filtrates were concentrated under reduced pressure to dryness. The crude was purified by flash column chromatography (dichloromethane/methanol=15/1) to give the title compound 238 (470 mg) as a brown oil.

### N¹-(2-(dimethylamino)ethyl)-N¹,N²,N²-trimethyl-N⁴-(4-(5-methyl-1H-indol-3-yl)-7H-pyrrolo[2,3-d]pyrimidin-2-yl)benzene-1,2,4-triamine (239)

NaOH (316.8 mg, 7.92 mmol) was added into a solution of compound 238 (470 mg, 0.74 mmol) in methanol (8 mL). The reaction mixture was stirred at 50° C. for 5 h. Solvent was removed in vacuo followed by addition of acetic acid. The mixture was extracted with dichloromethane. The organic layers were separated, dried over anhydrous Na₂SO₄ and then concentrated to dryness. The crude was purified by preparative thin layer chromatography to give the title compound 239 (30 mg, yield 9.8% over two steps) as a light-yellow solid.

### Procedures for the Synthesis of 244

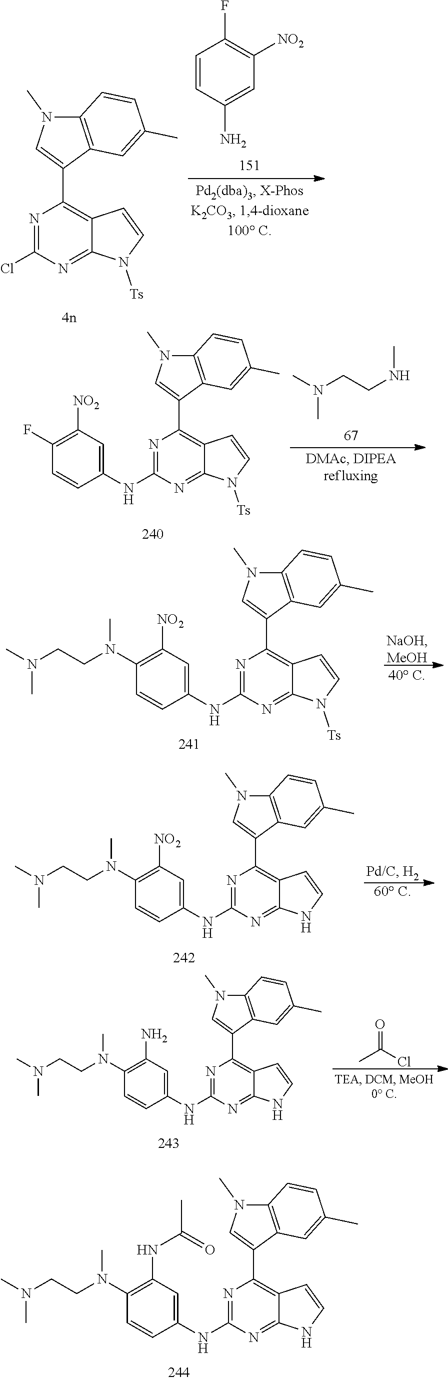

### 4-(1,5-dimethyl-1H-indol-3-yl)-N-(4-fluoro-3-nitrophenyl)-7-tosyl-7H-pyrrolo[2,3-d]pyrimidin-2-amine (240)

X-Phos (0.1 g, 0.21 mmol), Pd₂(dba)₃ (0.1 g, 0.11 mmol), K₂CO₃ (1.22 g, 8.8 mmol) and intermediate 4n (1.97 g, 4.3 mmol) was added to a stirred solution of 4-fluoro-3-nitroaniline 151 (0.61 g, 3.9 mmol) in 1,4-dioxane (40 ml). The resulting mixture was heated at 100° C. for 4 h. Then the reaction mixture was allowed to cool down. Water (100 ml) was added into the reaction mixture. The suspension was filtered and the filter cake was dried in vacuo to give the title compound 240 (1.9 g, yield 85.3%).

### N⁴-(4-(1,5-dimethyl-1H-indol-3-yl)-7-tosyl-7H-pyrrolo[2,3-d]pyrimidin-2-yl)-N¹-(2-(dimethylamino)ethyl)-N¹-methyl-2-nitrobenzene-1,4-diamine (241)

N′,N′,N-Trimethylethane-1,2-diamine 67 (0.71 g, 5.0 mmol) was added to a mixture of compound 240 (1.9 g, 3.3 mmol) and N, N-diisopropylethylamine (0.81 g, 6.2 mmol) in N,N-dimethyl acetamide (20 mL). The resulting mixture was heated at refluxing temperature for 3.5 h. Then the reaction mixture was allowed to cool down and quench with water (100 ml). The mixture was extracted with ethyl acetate (50 ml*3). The organic layers were combined, washed with brine (100 ml*3), and then concentrated under reduced pressure to give the title compound 241, which was used for next step without further purification. LC-MS: m/z 653.5 [M+H]⁺.

### N⁴-(4-(1,5-dimethyl-1H-indol-3-yl)-7H-pyrrolo[2,3-d]pyrimidin-2-yl)-N¹-(2-(dimethylamino)ethyl)-N¹-methyl-2-nitrobenzene-1,4-diamine (242)

A mixture of the above compound 241 and NaOH (3.7 g, 92.5 mmol) in methanol (30 ml) was stirred at 40° C. for 0.5 h. Then the reaction mixture was allowed to cool down and quench with water (100 ml). The mixture was extracted with dichloromethane (50 ml*3). The organic layers were combined, washed with brine (100 ml*3), and then concentrated under reduced pressure to give the title compound 242, which was used for next step without further purification. LC-MS: m/z 499.4 [M+H]⁺.

### N⁴-(4-(1,5-dimethyl-1H-indol-3-yl)-7H-pyrrolo[2,3-d]pyrimidin-2-yl)-N¹-(2-(dimethylamino)ethyl)-N¹-methylbenzene-1,2,4-triamine (243)

The above compound 242 was mixed with 5% Pd/C (50 mg, 0.24 mmol) in THF (50 ml). The resulting mixture was heated at 60° C. overnight under hydrogen atmosphere. The reaction mixture was filtered and concentrated. The residue was purified by flash column chromatography (dichloromethane/methanol=10/1) to give the compound 243 (0.3 g, yield 19.4% over three steps). LC-MS: m/z 469.6 [M+H]⁺

### N-(5-((4-(1,5-dimethyl-1H-indol-3-yl)-7H-pyrrolo[2,3-d]pyrimidin-2-yl)amino)-2-((2-(dimethylamino)ethyl)(methyl)amino)phenyl)acetamide (244)

The compound 243 (0.3 g, 0.6 mmol), triethylamine (0.15 g, 1.5 mmol), methanol (10 ml) and dichloromethane (10 ml) was added to the reaction flask (100 ml). The resulting mixture was stirred at 0° C. Acetyl chloride (0.07 g, 0.73 mmol) was added to the flask, and then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by flash column chromatography (dichloromethane/methanol=20/1) to give the compound 244 (140 mg, yield 45.7%). LC-MS: m/z 511.5 [M+H]⁺. ¹H NMR (500 MHz, Chloroform-d) δ 10.41 (s, 1H), 10.07 (s, 1H), 8.83 (s, 1H), 8.41 (s, 1H), 7.78 (s, 1H), 7.51 (d, J=10.5 Hz, 1H), 7.42 (s, 1H), 7.30-7.19 (m, 1H), 7.17-7.06 (m, 2H), 6.74 (d, J=2.7 Hz, 1H), 6.53 (d, J=3.1 Hz, 1H), 3.80 (s, 3H), 2.88-2.79 (m, 2H), 2.64 (s, 3H), 2.50 (s, 3H), 2.30-2.26 (m, 2H), 2.25 (s, 6H), 2.19 (s, 3H).

### Procedure for the Synthesis of 246

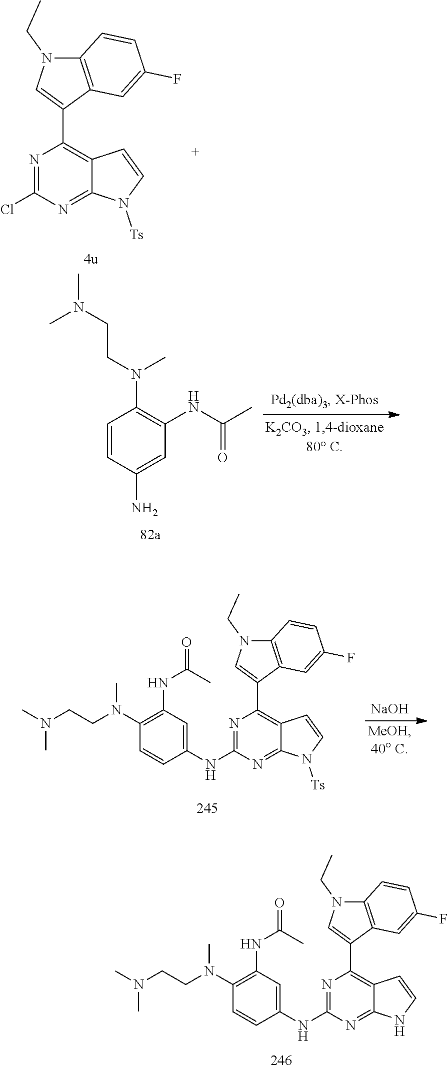 [SMILES: CCn1cc(-c2nc(Cl)nc3c2ccn3S(=O)(=O)c2ccc(C)cc2)c2cc(F)ccc21 ; CC(=O)Nc1cc(N)ccc1N(C)CCN(C)C ; CCn1cc(-c2nc(Nc3ccc(N(C)CCN(C)C)c(NC(C)=O)c3)nc3c2ccn3S(=O)(=O)c2ccc(C)cc2)c2cc(F)ccc21 ; "NaOH" ; CCn1cc(-c2nc(Nc3ccc(N(C)CCN(C)C)c(NC(C)=O)c3)nc3[nH]ccc23)c2cc(F)ccc21]

### N-(2-((2-(dimethylamino)ethyl)(methyl)amino)-5-((4-(1-ethyl-5-fluoro-1H-indol-3-yl)-7-tosyl-7H-pyrrolo[2,3-d]pyrimidin-2-yl)amino)phenyl)acetamide (245)

4u (102 mg, 0.22 mmol), 82a (51 mg, 0.20 mmol), Pd₂(dba)₃ (27 mg, 0.030 mmol), X-phos (57 mg, 0.12 mmol), K₂CO₃ (100 mg, 0.72 mmol) and dioxane (10 mL) were added into the flask. The mixture was stirred at 80° C. for 40 h. The reaction was then cooled to room temperature, filtered through Celite® and concentrated under reduced pressure to give a residue. The residue was purified by flash column chromatography (dichloromethane/methanol=20/1˜10/1, 1% Et₃N) to afford 245 (40 mg, yield 26.9%) as a white solid. MS-ESI (M+H)⁺: 683.4

### N-(2-((2-(dimethylamino)ethyl)(methyl)amino)-5-((4-(1-ethyl-5-fluoro-1H-indol-3-yl)-7H-pyrrolo[2,3-d]pyrimidin-2-yl)amino)phenyl)acetamide (246)

NaOH (30 mg, 0.75 mmol) was added into a solution of 245 (40 mg, 0.059 mmol) in methanol (10 mL) at 20° C. The mixture was stirred at 40° C. for 1 h. The reaction was then cooled to room temperature, and diluted with dichloromethane (100 mL). The organic phase was washed with saturated NH₄Cl, saturated NaHCO₃ and brine. The organic layer was separated, dried over Na₂SO₄, filtered and concentrated under reduced pressure to give a residue. The residue was purified by preparative thin layer chromatography (dichloromethane/methanol=10/1) to afford 246 (12 mg, yield 38.8%) as brown solid. ¹H NMR (500 MHz, CDCl₃) δ 9.96 (s, 1H), 9.58 (s, 1H), 8.89 (s, 1H), 8.26 (d, J=9.0 Hz, 2H), 8.01 (s, 1H), 7.36 (d, J=7.3 Hz, 1H), 7.30 (dd, J=8.9, 4.3 Hz, 2H), 7.09-6.98 (m, 3H), 6.60 (d, J=3.0 Hz, 1H), 4.26 (dd, J=14.7, 7.4 Hz, 2H), 3.19 (m, 2H), 2.93 (m, 2H), 2.66 (m, 9H), 2.41 (s, 3H), 1.55 (t, J=7.3 Hz, 3H). MS-ESI (M+H)⁺: 529.7

### Procedure for the Syntheses of 248a and 248b

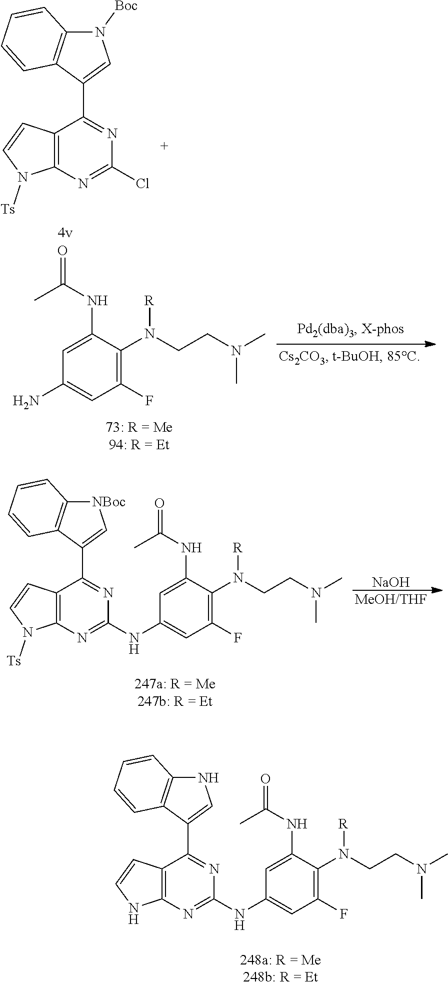

### tert-butyl 3-(2-((3-acetamido-4-((2-(dimethylamino)ethyl)(methyl)amino)-5-fluorophenyl)amino)-7-tosyl-7H-pyrrolo[2,3-d]pyrimidin-4-yl)-1H-indole-1-carboxylate (247a)

To the mixture of 4v (0.5 g, 0.96 mmol) and 73 (254 mg, 0.95 mmol) in t-BuOH (20 mL) were added Cs₂CO₃ (0.62 g, 1.91 mmol), Pd₂(dba)₃ (87 mg, 0.095 mmol) and X-phos (67 mg, 0.14 mmol). The mixture was stirred at 85° C. for 8 h. After cooling to room temperature, the mixture was concentrated and the residue was purified by flash column chromatography (dichloromethane/methanol=100/1-20/1) to give 247a (310 mg, yield 42.96%) as a brown solid. MS-ESI (M+H)⁺: 755.4

### N-(5-((4-(1H-indol-3-yl)-7H-pyrrolo[2,3-d]pyrimidin-2-yl)amino)-2-((2-(dimethylamino)ethyl)(methyl)amino)-3-fluorophenyl)acetamide (248a)

To a solution of 247a (300 mg, 0.40 mmol) in methanol (15 mL) and THF (15 mL) and H₂O (5 mL) was added NaOH (317 mg, 7.95 mmol). The mixture was stirred at 50° C. for 5 h. The mixture was concentrated and acidified to pH=7 with 1N HCl aqueous solution. The residue was purified by reversed phase flash column chromatography and lyophilized to give the 248a (70.45 mg, yield 35.41%) as a brown solid. MS-ESI (M+H)⁺:501.2. ¹H NMR (400 MHz, DMSO-d6) δ 11.75 (d, J=2.1 Hz, 1H), 11.49 (s, 1H), 10.34 (s, 1H), 9.39 (s, 1H), 8.91 (d, J=7.8 Hz, 1H), 8.36 (d, J=2.9 Hz, 1H), 8.18 (s, 1H), 8.01 (dd, J=15.6, 2.3 Hz, 1H), 7.47 (d, J=7.8 Hz, 1H), 7.28-7.07 (m, 3H), 6.84 (dd, J=3.5, 1.8 Hz, 1H), 3.07-2.94 (m, 2H), 2.76 (d, J=0.9 Hz, 3H), 2.23 (s, 8H), 2.08 (s, 3H).

### tert-butyl 3-(2-((3-acetamido-4-((2-(dimethylamino)ethyl)(ethyl)amino)-5-fluorophenyl)amino)-7-tosyl-7H-pyrrolo[2,3-d]pyrimidin-4-yl)-1H-indole-1-carboxylate (247b)

To the mixture of 4v (0.5 g, 0.96 mmol) and 94 (267 mg, 0.95 mmol) in t-BuOH (20 mL) were added Cs₂CO₃ (0.62 g, 1.91 mmol), Pd₂(dba)₃ (87 mg, 0.095 mmol) and X-phos (67 mg, 0.14 mmol). The mixture was stirred at 85° C. for 8 h. After cooling to room temperature and concentration, the residue was purified by flash column chromatography (dichloromethane/methanol=100/1-20/1) to give 247b (280 mg, 0.36 mmol, 38.09% yield) as a brown solid. MS-ESI (M+H)⁺: 769.3.

### N-(5-((4-(1H-indol-3-yl)-7H-pyrrolo[2,3-d]pyrimidin-2-yl)amino)-2-((2-(dimethylamino)ethyl)(methyl)amino)-3-fluorophenyl)acetamide (248b)

To a solution of 247b (260 mg, 0.33 mmol) in methanol (15 mL) and THF (15 mL) and H₂O (5 mL) was added NaOH (270 mg, 6.76 mmol). The mixture was stirred at 50° C. for 5 h. The mixture was concentrated and acidified to pH=7 with 1N HCl aqueous solution. The residue was purified by prep-HPLC and lyophilized to give the 248b (32.35 mg, yield 15.56%) as a brown solid. MS-ESI (M+H)⁺:515.3. ¹H NMR (400 MHz, DMSO-d6) δ 11.75 (s, 1H), 11.49 (s, 1H), 10.38 (s, 1H), 9.39 (s, 1H), 8.90 (d, J=7.8 Hz, 1H), 8.36 (d, J=2.9 Hz, 1H), 8.15 (s, 1H), 7.99 (dd, J=15.5, 2.3 Hz, 1H), 7.47 (d, J=7.9 Hz, 1H), 7.18-7.13 (m, 3H), 6.85 (dd, J=3.5, 1.8 Hz, 1H), 3.05 (d, J=7.2 Hz, 4H), 2.22 (s, 8H), 2.07 (s, 3H), 0.89 (t, J=7.1 Hz, 3H).

### Procedures for the Synthesis of 253

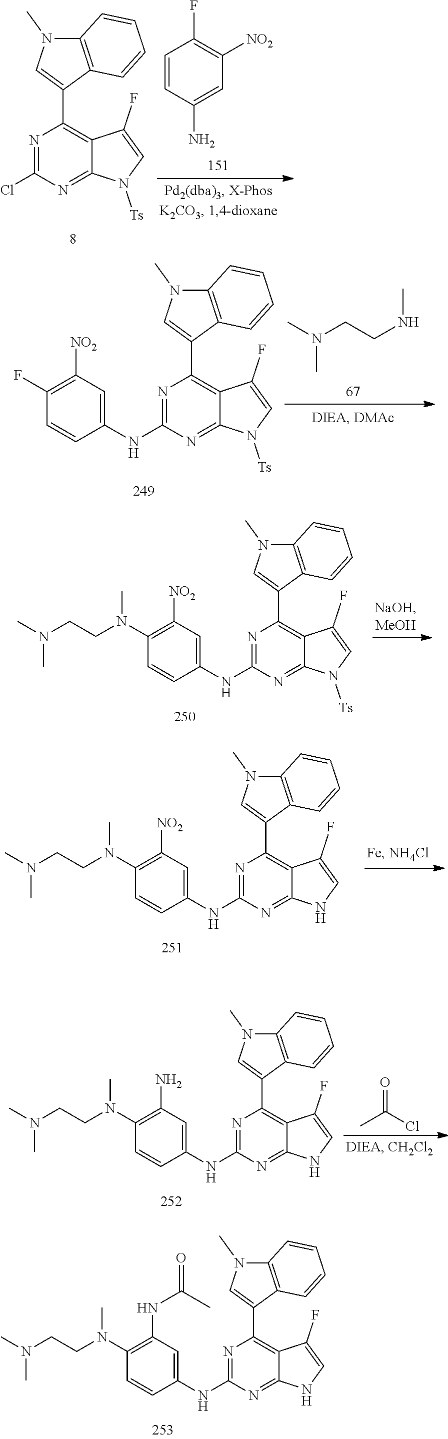

### 5-fluoro-N-(4-fluoro-3-nitrophenyl)-4-(1-methyl-1H-indol-3-yl)-7-tosyl-7H-pyrrolo[2,3-d]pyrimidin-2-amine (249)

A mixture of intermediate 8 (0.50 g, 1.10 mmol), intermediate 151 (0.15 g, 0.96 mmol), Pd₂(dba)₃ (0.20 g, 0.22 mmol), X-Phos (0.20 g, 0.42 mmol) and K₂CO₃ (1.0 g, 7.24 mmol) in 1,4-dioxane (20 mL) was stirred under N₂ at refluxing temperature for 2.5 h. After cooling to room temperature, the reaction mixtures was filtered through Celite® and the filtrates were concentrated under reduced pressure to dryness to give the crude title compound 249 as a red solid, which was used in the next step without further purification.

### N¹-(2-(dimethylamino)ethyl)-N⁴-(5-fluoro-4-(1-methyl-1H-indol-3-yl)-7-tosyl-7H-pyrrolo[2,3-d]pyrimidin-2-yl)-N¹-methyl-2-nitrobenzene-1,4-diamine (250)

N,N-diisopropyl ethylamine (1.5 g, 11.61 mmol) was added to a solution of compound 249 (0.6 g, 1.05 mmol) and N′-ethyl-N,N-dimethyl ethylenediamine 67 (0.6 g, 5.87 mmol) in N,N-dimethylacetamide (30 ml) at room temperature. The mixture was stirred at 90° C. for 4.5 h then cooled to room temperature. Water (60 ml) was added and the mixture was extracted with ethyl acetate (100 mL). The organic layer was separated, washed with water for 3 times, dried over Na₂SO₄, filtered and concentrated to dryness. The crude was purified by flash column chromatography (eluting with methanol/dichloromethane, from 1/10 to 1/5, v/v) to give the title compound 250 (0.6246 g, yield 90.6%) as a red oil.

### N¹-(2-(dimethylamino)ethyl)-N⁴-(5-fluoro-4-(1-methyl-1H-indol-3-yl)-7H-pyrrolo[2,3-d]pyrimidin-2-yl)-N¹-methyl-2-nitrobenzene-1,4-diamine (251)

NaOH (2.0 g, 50 mmol) was added into a solution of compound 250 (0.6246 g, 0.95 mmol) in methanol (10 mL). The reaction mixture was stirred at 50° C. for 0.5 h. Solvent was removed in vacuo. The mixture was extracted with dichloromethane. The organic layers were separated, dried over anhydrous Na₂SO₄ and then concentrated to dryness. The crude was purified by flash column chromatography to give the title compound 251 (300 mg, yield 62.8%) as a red oil.

### N¹-(2-(dimethylamino)ethyl)-N⁴-(5-fluoro-4-(1-methyl-1H-indol-3-yl)-7H-pyrrolo[2,3-d]pyrimidin-2-yl)-N¹-methylbenzene-1,2,4-triamine (252)

A mixture of Compound 251 (300 mg, 0.60 mmol), iron powder (150 mg, 2.67 mmol) and NH₄Cl (300 mg, 5.61 mmol) in methanol/H₂O (15 mL/3 mL) was stirred at refluxing temperature for 2 h. The reaction mixture was cooled to room temperature and filtered through Celite®. The filter cake was washed with methanol. The filtrate was concentrated, then extracted with ethyl acetate (20 mL×3). The combined organic layer was concentrated to give crude product 252 as a gray solid (150 mg, yield in 53.2%) which was used directly in the next step.

### N-(2-((2-(dimethylamino)ethyl)(methyl)amino)-5-((5-fluoro-4-(1-methyl-1H-indol-3-yl)-7H-pyrrolo[2,3-d]pyrimidin-2-yl)amino)phenyl)acetamide (253)

Acetyl chloride (37.6 mg, 0.48 mmol) was added to the mixture of compound 252 (150 mg, 0.32 mmol) and N,N-diisopropyl ethylamine (87 mg, 0.67 mmol) in dichloromethane (15 ml) at 0° C. The reaction mixture was stirred for 0.5 h, and then quenched with H₂O. The organic layers were combined, and concentrated under reduced pressure. The residue was purified by flash column chromatography (dichloromethane/methanol=20/1) to give the title compound 253 (22 mg, yield 13.5%).

### Procedure for the Syntheses of 255a and 255b

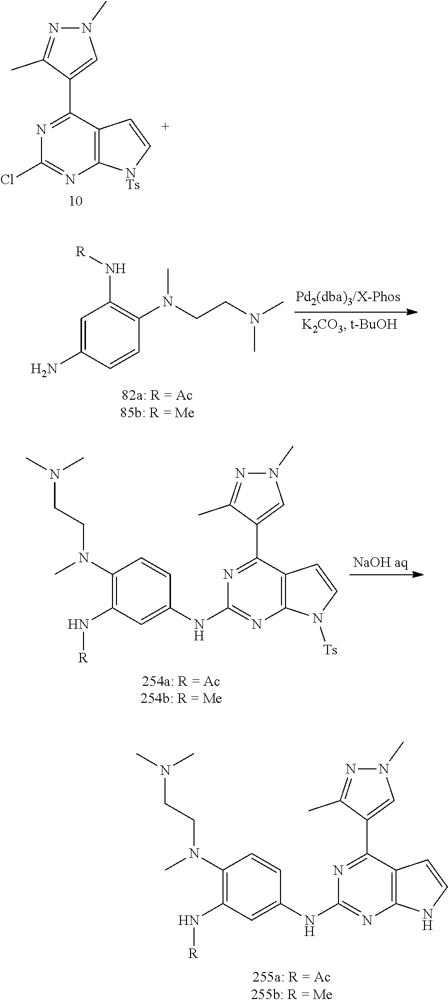

### N-(5-((4-(1,3-dimethyl-1H-pyrazol-4-yl)-7-tosyl-7H-pyrrolo[2,3-d]pyrimidin-2-yl)amino)-2-((2-(dimethylamino)ethyl)(methyl)amino)phenyl)acetamide (254a)

To the mixture of intermediate 10 (0.200 g, 0.5 mmol) and intermediate 82a (0.125 g, 0.5 mmol) in t-BuOH (20 mL) were added K₂CO₃ (0.150 g, 1.2 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) to give crude product 254a (50 mg).

### N-(5-(4-(1,3-dimethyl-1H-pyrazol-4-yl)-7H-pyrrolo[2,3-d]pyrimidin-2-ylamino)-2-((2-(dimethylamino)ethyl)(methyl)amino)phenyl)acetamide (255a)

The crude product 254a was stirred in 1N NaOH (5 mL) and methanol (5 mL) at 40° C. for 3 h. After cooling to room temperature and concentration, the resulting mixture was extracted by dichloromethane. The organic layer was washed with brine and concentrated. The residue was purified by preparative thin layer chromatography to give 255a (20 mg, 8.7% yield over two steps) as a yellow solid. LC-MS: m/z 462.8 [M+H]⁺.

### N⁴-(4-(1,3-dimethyl-1H-pyrazol-4-yl)-7-tosyl-7H-pyrrolo[2,3-d]pyrimidin-2-yl)-N¹-(2-(dimethylamino)ethyl)-N¹,N²-dimethylbenzene-1,2,4-triamine (254b)

To the mixture of intermediate 10 (0.200 g, 0.5 mmol) and intermediate 85b (0.125 g, 0.56 mmol) in t-BuOH (20 mL) were added K₂CO₃ (0.150 g, 1.2 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) to give crude product 254b (40 mg).

### N-(5-(4-(1,3-dimethyl-1H-pyrazol-4-yl)-7H-pyrrolo[2,3-d]pyrimidin-2-ylamino)-2-((2-(dimethylamino)ethyl)(methyl)amino)phenyl)acetamide (255b)

The crude product 254b was then stirred in 1N NaOH (5 mL) and methanol (5 mL) at 40° C. for 3 h. After cooling to room temperature and concentration, the residue was extracted by dichloromethane. The organic layer was washed with brine and concentrated. The residue was purified by preparative thin layer chromatography to give 255b (10 mg, 4.6% yield over two steps) as a yellow solid. LC-MS: m/z 434.3 [M+H]⁺.

### Procedure for the Syntheses of 256a-d

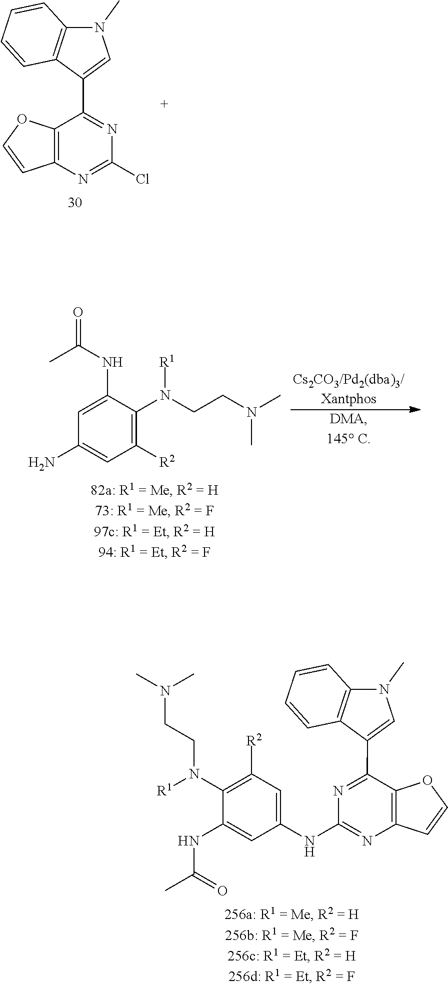

### N-(2-((2-(dimethylamino)ethyl)(methyl)amino)-5-((4-(1-methyl-1H-indol-3-yl)furo[3,2-d]pyrimidin-2-yl)amino)phenyl)acetamide (256a)

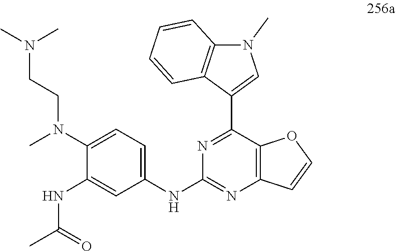 [SMILES: CC(=O)Nc1cc(Nc2nc(-c3cn(C)c4ccccc34)c3occc3n2)ccc1N(C)CCN(C)C]

To a solution of 30 (375 mg, 1.33 mmol) in 10 mL of DMA was added 82a (365 mg, 1.46 mmol), Pd₂ (dba)₃ (122 mg, 0.13 mmol), Xant-Phos(116 mg, 0.199 mmol) and Cs₂CO₃ (650 mg, 1.99 mmol). The mixture was stirred at 120° C. under Ar overnight. After cooling to room temperature, the mixture was poured into water (50 mL). The aqueous phase was extracted with ethyl acetate (200 mL*2) and washed with brine (50 mL*3). The organic layers were dried over Na₂SO₄, filtered and concentrated under reduced pressure. The residue was purified by Pre-HPLC (MeCN/0.05% NH4OH in water from 0 to 100%) to afford 256a (127 mg, yield 19.3%) as a yellow solid. MS-ESI (M+H)⁺: 498.2. ¹H NMR (400 MHz, DMSO-d6) δ 10.02 (s, 1H), 9.30 (s, 1H), 8.89 (d, J=8.0 Hz, 1H), 8.52-8.50 (m, 2H), 8.33 (s, 1H), 7.68 (d, J=7.2 Hz, 1H), 7.58 (d, J=8.0 Hz, 1H), 7.33 (t, J=7.6 Hz, 1H), 7.26-7.24 (m, 2H), 6.95 (s, 1H), 3.98 (s, 3H), 2.84-2.83 (m, 2H), 2.68 (s, 3H), 2.30 (br, 2H), 2.24 (s, 6H), 2.09 (s, 3H).

### N-(2-((2-(dimethylamino) ethyl) (methyl) amino)-3-fluoro-5-((4-(1-methyl-1H-indol-3-yl) furo[3, 2-d]pyrimidin-2-yl)amino)phenyl) acetamide (256b)

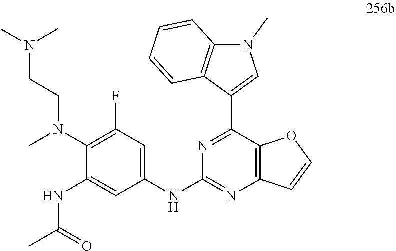 [SMILES: CC(=O)Nc1cc(Nc2nc(-c3cn(C)c4ccccc34)c3occc3n2)cc(F)c1N(C)CCN(C)C]

Synthesis of 256b was similar to that of 256a. The crude was purified by Pre-HPLC (MeCN/0.05% NH4OH in water from 0 to 100%) to afford 256b (144 mg, yield 23%) as a yellow solid. MS-ESI (M+H)⁺: 516.2. ¹H NMR (400 MHz, DMSO-d6) δ 10.35 (s, 1H), 9.60 (s, 1H), 8.92 (d, J=8.0 Hz, 1H), 8.49 (s, 1H), 8.34 (s, 1H), 8.28 (s, 1H), 7.82 (d, J=15.2 Hz, 1H), 7.57 (d, J=8.0 Hz, 1H), 7.33-7.31 (m, 1H), 7.26-7.23 (m, 1H), 7.00 (d, J=2.0 Hz, 1H), 3.96 (s, 3H), 2.99 (br, 2H), 2.75 (s, 3H), 2.21 (br, 8H), 2.07 (s, 3H).

### N-(2-((2-(dimethylamino)ethyl)(ethyl)amino)-5-((4-(1-methyl-1H-indol-3-yl)furo[3,2-d]pyrimidin-2-yl)amino)phenyl)acetamide (256c)

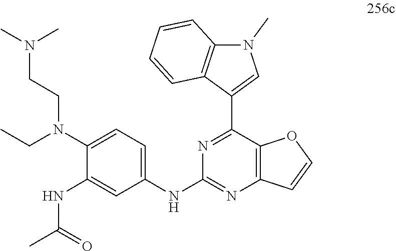 [SMILES: CCN(CCN(C)C)c1ccc(Nc2nc(-c3cn(C)c4ccccc34)c3occc3n2)cc1NC(C)=O]

Synthesis of 256c was similar to that of 256a. The crude was purified by Pre-HPLC (MeCN/0.05% NH4OH in water from 0 to 100%) to afford 256c (160 mg, yield 22%) as a yellow solid. MS-ESI (M+H)⁺: 512.2. ¹H NMR (400 MHz, DMSO-d6) δ 10.10 (s, 1H), 9.32 (s, 1H), 8.88 (d, J=7.6 Hz, 1H), 8.52-8.50 (mz, 2H), 8.32 (d, J=2.0 Hz, 1H), 7.70 (d, J=8.4 Hz, 1H), 7.57 (d, J=8.0 Hz, 1H), 7.31 (d, J=7.2 Hz, 1H), 7.23-7.20 (m, 2H), 6.95 (d, J=2.0 Hz, 1H), 3.97 (s, 3H), 3.01-2.95 (m, 2H), 2.93 (t, J=5.6 Hz, 2H), 2.25-2.13 (m, 8H), 2.06 (s, 3H), 0.88 (t, J=7.2 Hz, 3H).

### N-(2-((2-(dimethylamino)ethyl)(ethyl)amino)-3-fluoro-5-((4-(1-methyl-1H-indol-3-yl)furo[3,2-d]pyrimidin-2-yl)amino)phenyl)acetamide (256d)

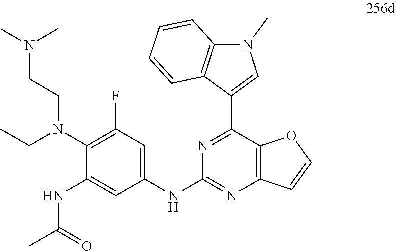 [SMILES: CCN(CCN(C)C)c1c(F)cc(Nc2nc(-c3cn(C)c4ccccc34)c3occc3n2)cc1NC(C)=O]

Synthesis of 256d was similar to that of 256a. The crude was purified by Pre-HPLC (MeCN/0.05% NH4OH in water from 0 to 100%) to afford 256d (133 mg, yield 22%) as a yellow solid. MS-ESI (M+H)⁺: 530.2. ¹H NMR (400 MHz, DMSO-d6) δ 10.40 (s, 1H), 9.62 (s, 1H), 8.92 (d, J=7.6 Hz, 1H), 8.51 (s, 1H), 8.35 (d, J=2.0 Hz, 1H), 8.27 (s, 1H), 7.83 (d, J=15.2 Hz, 1H), 7.59 (d, J=8.0 Hz, 1H), 7.34-7.31 (m, 1H), 7.26-7.24 (m, 1H), 7.02 (d, J=2.0 Hz, 1H), 3.98 (s, 3H), 3.06 (br, 4H), 2.22 (br, 8H), 2.07 (s, 3H), 0.90 (t, J=7.2 Hz, 3H).

### Procedure for the Syntheses of 257a-d

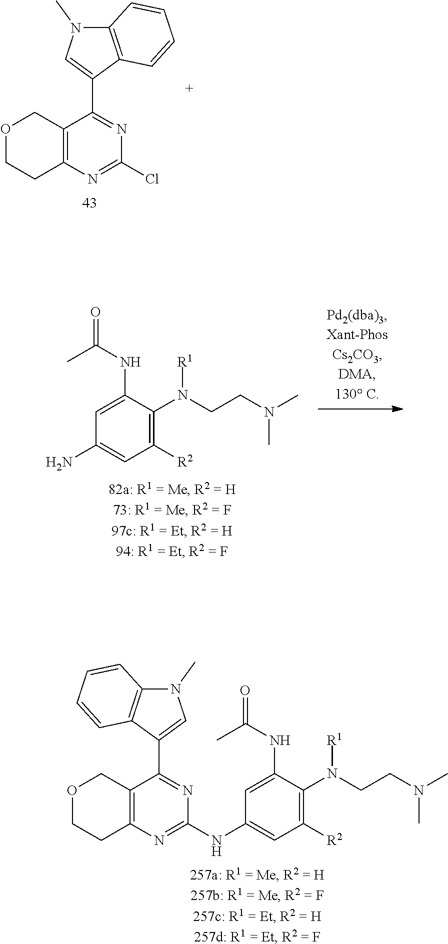

### N-(2-((2-(dimethylamino)ethyl)(methyl)amino)-5-((4-(1-methyl-1H-indol-3-yl)-7,8-dihydro-5H-pyrano[4,3-d]pyrimidin-2-yl)amino)phenyl)acetamide (257a)

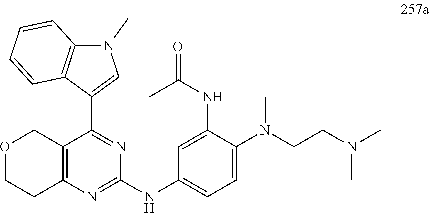 [SMILES: CC(=O)Nc1cc(Nc2nc3c(c(-c4cn(C)c5ccccc45)n2)COCC3)ccc1N(C)CCN(C)C]

Synthesis of 257a was similar to that of 256a. The crude was purified by Pre-HPLC (MeCN/0.05% NH4OH in water from 0 to 100%) to afford 257a (94 mg, yield 28.2%) as a yellow solid. MS-ESI (M+H)⁺: 514.3. ¹H NMR (400 MHz, DMSO) δ 9.96 (s, 1H), 9.22 (s, 1H), 8.59 (d, J=8.1 Hz, 1H), 8.50 (s, 1H), 7.79 (s, 1H), 7.57 (dd, J=8.7, 2.5 Hz, 1H), 7.49 (d, J=8.2 Hz, 1H), 7.25 (t, J=7.1 Hz, 1H), 7.16 (d, J=8.7 Hz, 1H), 7.11 (t, J=7.3 Hz, 1H), 4.80 (s, 2H), 4.00 (t, J=5.8 Hz, 2H), 3.87 (s, 3H), 2.80 (dd, J=14.0, 6.0 Hz, 4H), 2.64 (s, 3H), 2.26 (t, J=5.6 Hz, 2H), 2.21 (s, 6H), 2.04 (s, 3H).

### N-(2-((2-(dimethylamino)ethyl)(methyl)amino)-3-fluoro-5-((4-(1-methyl-1H-indol-3-yl)-7,8-dihydro-5H-pyrano[4,3-d]pyrimidin-2-yl)amino)phenyl)acetamide (257b)

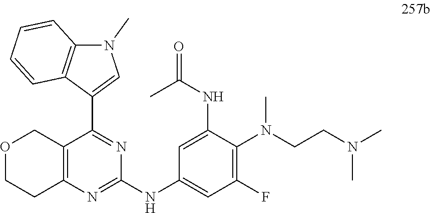 [SMILES: CC(=O)Nc1cc(Nc2nc3c(c(-c4cn(C)c5ccccc45)n2)COCC3)cc(F)c1N(C)CCN(C)C]

Synthesis of 257b was similar to that of 256a. The crude was purified by Pre-HPLC (MeCN/0.05% NH4OH in water from 0 to 100%) to afford 257b (50 mg, yield 28%) as a yellow solid. MS-ESI (M+H)⁺: 532.2. ¹H NMR (400 MHz, DMSO-d6) δ 10.33 (s, 1H), 9.53 (s, 1H), 8.61 (d, J=8.0 Hz, 1H), 8.28 (s, 1H), 7.80 (s, 1H), 7.73 (dd, J=15.2, 2.4 Hz, 1H), 7.50 (d, J=8.0 Hz, 1H), 7.26 (t, J=7.6 Hz, 1H), 7.15 (t, J=7.6 Hz, 1H), 4.81 (s, 2H), 4.01 (t, J=5.6 Hz, 2H), 3.88 (s, 3H), 2.98-2.93 (m, 2H), 2.84 (t, J=5.6 Hz, 2H), 2.72 (s, 3H), 2.20 (br, 8H), 2.04 (s, 3H).

### N-(2-((2-(dimethylamino)ethyl)(ethyl)amino)-5-((4-(1-methyl-1H-indol-3-yl)-7,8-dihydro-5H-pyrano[4,3-d]pyrimidin-2-yl)amino)phenyl)acetamide(257c)

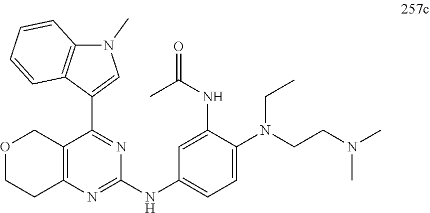 [SMILES: CCN(CCN(C)C)c1ccc(Nc2nc3c(c(-c4cn(C)c5ccccc45)n2)COCC3)cc1NC(C)=O]

Synthesis of 257c was similar to that of 256a. The crude was purified by Pre-HPLC (MeCN/0.05% NH4OH in water from 0 to 100%) to afford 257c (86 mg, yield 20.84%) as a yellow solid. MS-ESI (M+H)⁺: 528.3. ¹H NMR (400 MHz, DMSO) δ 10.08 (s, 1H), 9.26 (s, 1H), 8.61 (d, J=7.9 Hz, 1H), 8.53 (s, 1H), 7.81 (s, 1H), 7.61 (d, J=7.4 Hz, 1H), 7.51 (d, J=8.2 Hz, 1H), 7.26 (t, J=7.4 Hz, 1H), 7.19 (d, J=8.6 Hz, 1H), 7.12 (t, J=7.4 Hz, 1H), 4.83 (s, 2H), 4.02 (d, J=5.2 Hz, 2H), 3.90 (s, 3H), 2.99-2.88 (m, 4H), 2.84 (s, 2H), 2.19 (d, J=11.5 Hz, 8H), 2.05 (s, 3H), 0.86 (t, J=6.9 Hz, 3H).

### N-(2-((2-(dimethylamino)ethyl)(ethyl)amino)-3-fluoro-5-((4-(1-methyl-1H-indol-3-yl)-7,8-dihydro-5H-pyrano[4,3-d]pyrimidin-2-yl)amino)phenyl)acetamide (257d)

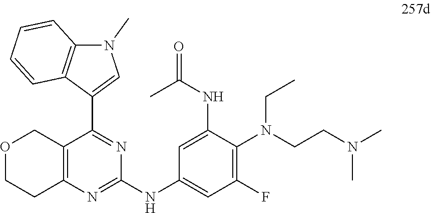 [SMILES: CCN(CCN(C)C)c1c(F)cc(Nc2nc3c(c(-c4cn(C)c5ccccc45)n2)COCC3)cc1NC(C)=O]

Synthesis of 257d was similar to that of 256a. The crude was purified by Pre-HPLC (MeCN/0.05% NH4OH in water from 0 to 100%) to afford 257d (46.5 mg, yield 21.25%) as a yellow solid. MS-ESI (M+H)⁺: 546.2. ¹H NMR (400 MHz, DMSO) δ 10.35 (s, 1H), 9.53 (s, 1H), 8.60 (d, J=8.0 Hz, 1H), 8.24 (s, 1H), 7.80 (s, 1H), 7.71 (dd, J=15.2, 2.2 Hz, 1H), 7.49 (d, J=8.2 Hz, 1H), 7.25 (t, J=7.5 Hz, 1H), 7.12 (t, J=7.5 Hz, 1H), 4.81 (s, 2H), 4.00 (t, J=5.7 Hz, 2H), 3.87 (s, 3H), 3.01 (d, J=7.0 Hz, 4H), 2.83 (t, J=5.6 Hz, 2H), 2.17 (d, J=10.1 Hz, 8H), 2.02 (s, 3H), 0.85 (t, J=7.1 Hz, 3H).

### Procedure for the Syntheses of 258a-d

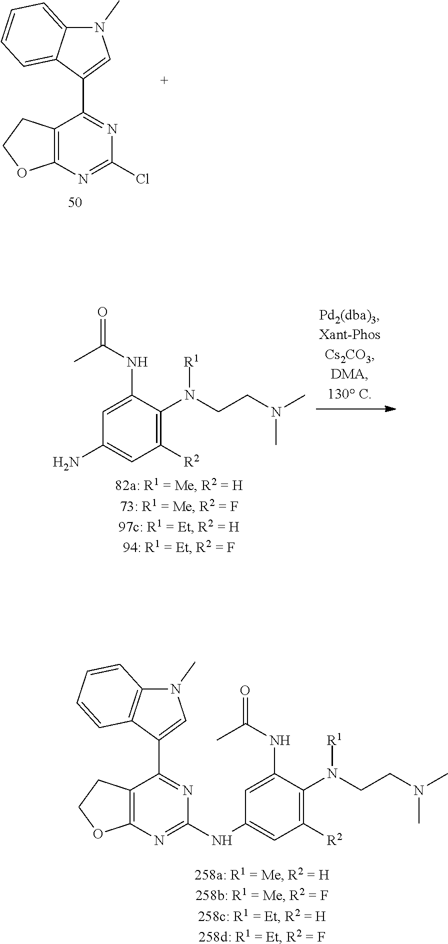

### N-(2-((2-(dimethylamino)ethyl)(methyl)amino)-5-((4-(1-methyl-1H-indol-3-yl)-5,6-dihydrofuro[2,3-d]pyrimidin-2-yl)amino)phenyl)acetamide (258a)

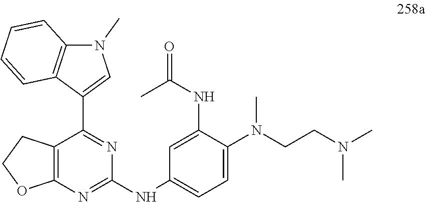 [SMILES: CC(=O)Nc1cc(Nc2nc3c(c(-c4cn(C)c5ccccc45)n2)CCO3)ccc1N(C)CCN(C)C]

To a solution of 50 (200.0 mg, 0.7 mmol) and 82a (175.23 mg, 0.7 mmol) in DMA (20 mL) was added Cs₂CO₃ (684.18 mg, 2.1 mmol), Pd₂(dba)₃ (64 mg, 0.07 mmol) and Xant-phos (40.5 mg, 0.07 mmol). The mixture was stirred at 130° C. for 12 h under N₂ atmosphere. The mixture was filtered and concentrated, the residue was purified by prep-HPLC to afford the desired product 258a (33.47 mg, yield 9.57%) as a brown solid. ¹H NMR (400 MHz, CD₃OD) δ 8.53 (d, J=2.4 Hz, 1H), 8.42 (d, J=8.0 Hz, 1H), 8.06 (s, 1H), 7.40-7.31 (m, 2H), 7.26 (d, J=8.6 Hz, 1H), 7.22-7.16 (m, 1H), 7.04-7.07 (m, 1H), 4.66 (t, J=8.8 Hz, 2H), 3.86 (s, 3H), 3.15 (t, J=8.8 Hz, 2H), 3.05 (t, J=6.0 Hz, 2H), 2.46 (t, J=5.9 Hz, 2H), 2.31 (s, 6H), 2.18 (s, 3H). MS-ESI (M+H)⁺:500.3;

### N-(2-((2-(dimethylamino)ethyl)(methyl)amino)-3-fluoro-5-((4-(1-methyl-1H-indol-3-yl)-5,6-dihydrofuro[2,3-d]pyrimidin-2-yl)amino)phenyl)acetamide (258b)

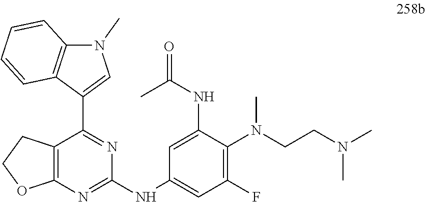 [SMILES: CC(=O)Nc1cc(Nc2nc3c(c(-c4cn(C)c5ccccc45)n2)CCO3)cc(F)c1N(C)CCN(C)C]

Synthesis of 258b was similar to that of 258a. The residue was purified by prep-HPLC (NH₃·H₂O) to afford the desired product 258b (23.39 mg, yield 6.46%) as a brown solid. ¹H NMR (400 MHz, CD₃OD) δ 8.46 (d, J=8.0 Hz, 1H), 8.39 (s, 1H), 8.10 (s, 1H), 7.40 (s, 2H), 7.21 (t, J=7.2 Hz, 1H), 7.08 (t, J=7.4 Hz, 1H), 4.69-4.65 (m, 2H), 3.87 (s, 3H), 3.24-3.14 (m, 4H), 2.83 (d, J=1.1 Hz, 3H), 2.40 (t, J=5.8 Hz, 2H), 2.32 (s, 6H), 2.18 (s, 3H); MS-ESI (M+H)⁺:518.4;

### N-(2-((2-(dimethylamino)ethyl)(ethyl)amino)-5-((4-(1-methyl-1H-indol-3-yl)-5,6-dihydrofuro[2,3-d]pyrimidin-2-yl)amino)phenyl)acetamide (258c)

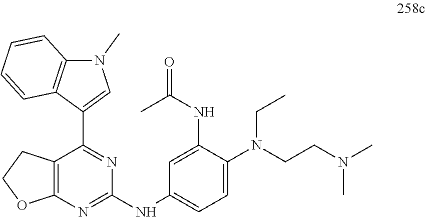 [SMILES: CCN(CCN(C)C)c1ccc(Nc2nc3c(c(-c4cn(C)c5ccccc45)n2)CCO3)cc1NC(C)=O]

Synthesis of 258c was similar to that of 258a. The residue was purified by prep-HPLC to afford the desired product 258c (24.38 mg, yield 6.78%) as a brown solid. ¹H NMR (400 MHz, CD₃OD) δ 8.27 (d, J=8.0 Hz, 1H), 8.14 (s, 1H), 7.95 (d, J=2.4 Hz, 1H), 7.47 (dd, J=9.0, 5.4 Hz, 2H), 7.35 (d, J=8.8 Hz, 1H), 7.29 (s, 1H), 7.15 (s, 1H), 4.90 (t, J=8.8 Hz, 2H), 3.91 (s, 3H), 3.53 (d, J=5.8 Hz, 2H), 3.23 (t, J=8.8 Hz, 2H), 3.15 (d, J=7.2 Hz, 2H), 2.86 (s, 6H), 2.20 (s, 3H), 1.07 (t, J=7.2 Hz, 3H); MS-ESI (M+H)⁺: 514.4.

### N-(2-((2-(dimethylamino)ethyl)(ethyl)amino)-3-fluoro-5-((4-(1-methyl-1H-indol-3-yl)-5,6-dihydrofuro[2,3-d]pyrimidin-2-yl)amino)phenyl)acetamide (258d)

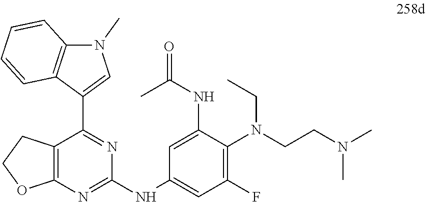 [SMILES: CCN(CCN(C)C)c1c(F)cc(Nc2nc3c(c(-c4cn(C)c5ccccc45)n2)CCO3)cc1NC(C)=O]

Synthesis of 258d was similar to that of 258a. The residue was purified by prep-HPLC (FA) to afford the desired product 258d (18.33 mg, yield 4.93%) as a brown solid. ¹H NMR (400 MHz, CD₃OD) δ 8.48 (d, J=8.0 Hz, 1H), 8.34 (s, 1H), 8.09 (s, 1H), 7.82 (s, 1H), 7.38 (d, J=8.4 Hz, 2H), 7.21 (s, 1H), 7.08 (s, 1H), 4.67 (t, J=8.4 Hz, 2H), 3.87 (s, 3H), 3.19 (t, J=8.4 Hz, 6H), 2.36 (s, 2H), 2.31 (s, 6H), 2.17 (s, 3H), 1.00 (t, J=7.0 Hz, 3H). MS-ESI (M+H)⁺:532.3;

### Synthesis of Compounds of Formula I

### Procedure for the Syntheses of 259a, 259b

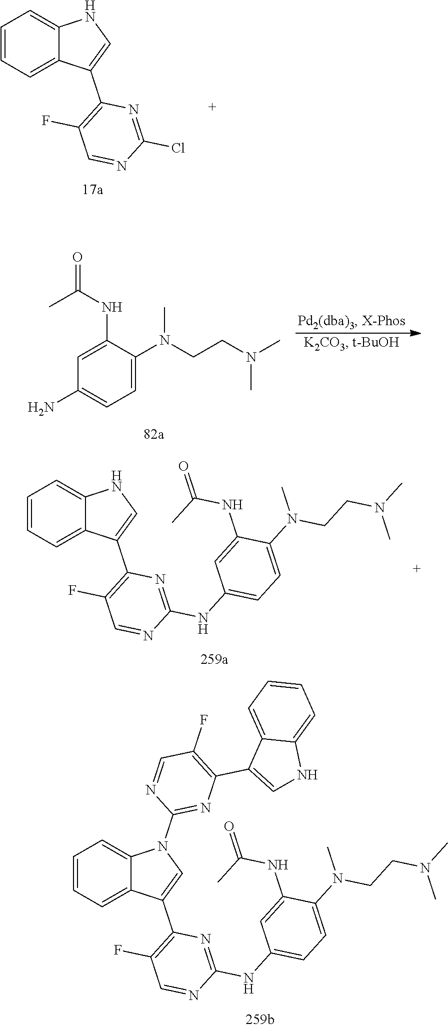

### N-(2-((2-(dimethylamino)ethyl)(methyl)amino)-5-((5-fluoro-4-(1H-indol-3-yl)pyrimidin-2-yl)amino)phenyl)acetamide (259a) & N-(2-((2-(dimethylamino)ethyl)(methyl)amino)-5-((5-fluoro-4-(1-(5-fluoro-4-(1H-indol-3-yl)pyrimidin-2-yl)-1H-indol-3-yl)pyrimidin-2-yl)amino)phenyl)acetamide (259b)

A mixture of 17a (0.21 g, 0.85 mmol), 82a (0.15 g, 0.60 mmol), Pd₂(dba)₃ (0.17 g, 0.19 mmol), X-Phos (0.18 g, 0.38 mmol) and K₂CO₃ (0.31 g 0.22 mmol) in t-BuOH (20 mL) was stirred at 95° C. for 2.5 h. After cooling to 45° C., the mixture was filtered off and the filtrate was concentrated under reduced pressure. The residue was dissolved in ethyl acetate (50 mL), and washed with H₂O (20 mL*3), dried with Na₂SO₄. After concentration, the residue was purified by flash column chromatography (dichloromethane/methanol=15/1) and preparative thin layer chromatography (dichloromethane/methanol=12/1) to give the title product 259a (25 mg, yield 9.1%, a gray solid) and 259b (22 mg, yield 5.5%, a gray solid) For 259a: (MS-ESI (M+H)⁺: 462.3. ¹H NMR (500 MHz, DMSO-d₆) δ 12.04 (s, 1H), 9.61 (s, 1H), 9.44 (s, 1H), 8.77-8.69 (m, 1H), 8.37 (d, J=3.9 Hz, 1H), 8.33 (s, 1H), 8.20-8.15 (m, 1H), 7.52 (dd, J=11.7, 5.3 Hz, 2H), 7.23 (t, J=8.0 Hz, 1H), 7.19 (dd, J=8.3, 2.9 Hz, 2H), 3.19 (s, 4H), 2.71 (s, 6H), 2.57 (s, 3H), 2.23 (s, 3H).) For 259b: (MS-ESI (M+H)⁺: 673.3)

### Procedure for the Synthesis of 260

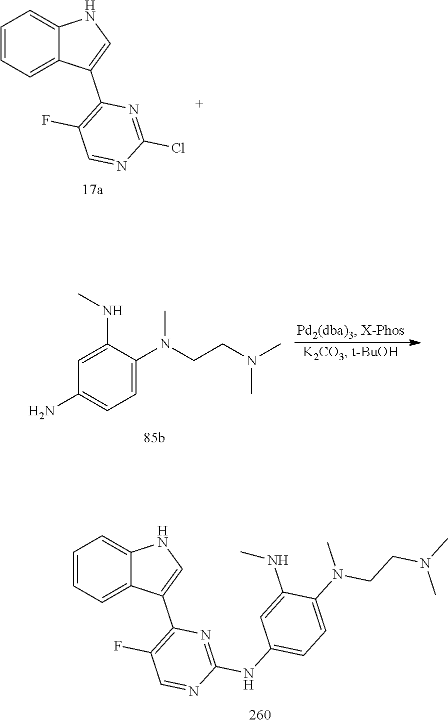

### N¹-(2-(dimethylamino)ethyl)-N⁴-(5-fluoro-4-(1H-indol-3-yl)pyrimidin-2-yl)-N¹,N²-dimethylbenzene-1,2,4-triamine (260)

Synthesis of 260 was similar to that of 259a. The residue was purified by flash column chromatography (dichloromethane/methanol=15/1) and preparative thin layer chromatography (dichloromethane/methanol=12/1) to give the title product 260 (8 mg, yield 1.47%) as a gray solid. LC-MS: m/z 434.4 [M+H]⁺. ¹H NMR (500 MHz, DMSO-d₆) δ 12.04 (s, 1H), 9.18 (s, 1H), 8.76 (d, J=8.0 Hz, 1H), 8.37 (d, J=3.8 Hz, 1H), 8.16 (d, J=3.2 Hz, 1H), 7.51 (d, J=8.1 Hz, 1H), 7.23 (t, J=7.6 Hz, 1H), 7.14 (t, J=7.5 Hz, 1H), 7.06-6.99 (m, 1H), 6.94 (d, J=8.4 Hz, 1H), 6.87 (d, J=2.4 Hz, 1H), 3.34 (s, 4H), 3.21-3.09 (m, 4H), 2.71 (s, 3H), 2.68 (s, 6H).

### Procedure for the Synthesis of 261

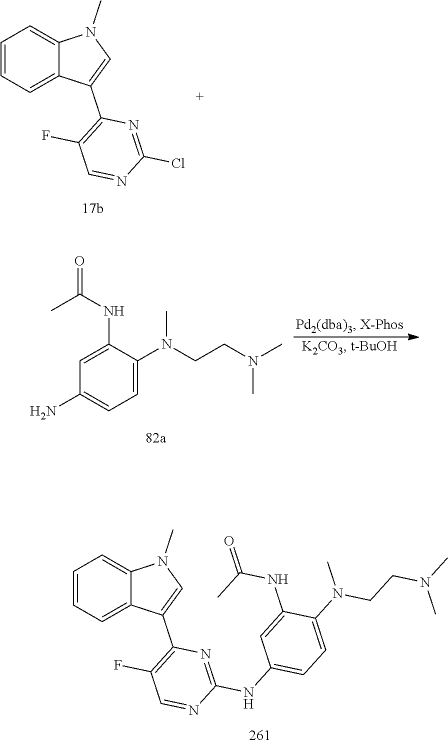

### N-(2-((2-(dimethylamino)ethyl)(methyl)amino)-5-((5-fluoro-4-(1-methyl-1H-indol-3-yl)pyrimidin-2-yl)amino)phenyl)acetamide (261)

Synthesis of 261 was similar to that of 259a. The residue was purified by preparative thin layer chromatography (dichloromethane/methanol=12/1) to give the desired product 261 (54 mg, yield 9.6%) as a gray solid. MS-ESI (M+H)⁺: 476.2. ¹H NMR (500 MHz, DMSO-d₆) δ 9.55 (s, 1H), 9.47 (s, 1H), 8.76 (d, J=8.0 Hz, 1H), 8.38 (d, J=3.9 Hz, 1H), 8.35 (d, J=2.4 Hz, 1H), 8.25 (d, J=2.9 Hz, 1H), 7.56 (d, J=8.2 Hz, 1H), 7.53 (dd, J=8.7, 2.6 Hz, 1H), 7.31 (ddd, J=8.1, 7.0, 1.2 Hz, 1H), 7.23 (ddd, J=8.1, 7.0, 1.1 Hz, 1H), 7.20 (d, J=8.7 Hz, 1H), 3.92 (s, 3H), 3.27-3.11 (m, 4H), 2.74 (s, 6H), 2.57 (s, 3H), 2.23 (s, 3H).

### Procedure for the Synthesis of 262

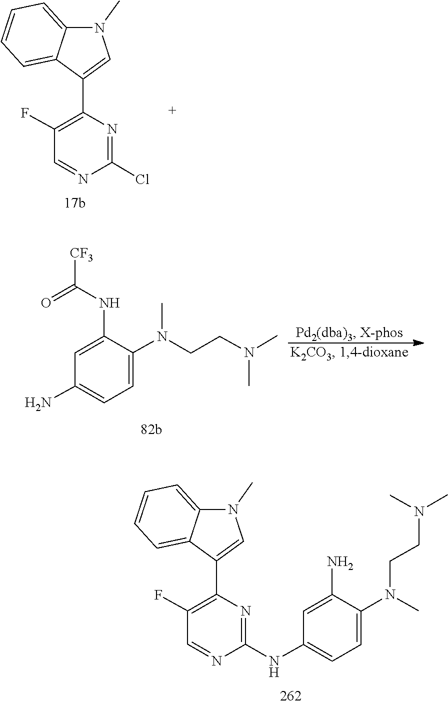 [SMILES: Cn1cc(-c2nc(Cl)ncc2F)c2ccccc21 ; CN(C)CCN(C)c1ccc(N)cc1NC(=O)C(F)(F)F ; CN(C)CCN(C)c1ccc(Nc2ncc(F)c(-c3cn(C)c4ccccc34)n2)cc1N]

### N¹-(2-(dimethylamino)ethyl)-N⁴-(5-fluoro-4-(1-methyl-1H-indol-3-yl)pyrimidin-2-yl)-N-methylbenzene-1,2,4-triamine (262)

X-Phos (0.02 g, 0.04 mmol), Pd₂(dba)₃ (0.02 g, 0.02 mmol), K₂CO₃ (0.16 g, 1.1 mmol) and 82b (0.17 g, 0.55 mmol) were added to a solution of 17b (0.15 g, 0.57 mmol) in 1,4-dioxane (40 ml). The resulting mixture was stirred at 90° C. for 4 hrs. Then the reaction mixture was cooled down and filtered. The filtrate was concentrated. The residue was purified by flash column chromatography (dichloromethane/methanol=20/1) to give the title compound 262 (65 mg, yield 22.3%). MS-ESI (M+H)⁺:530.22; ¹H NMR (500 MHz, DMSO-d6) δ ppm 9.14 (s, 1H), 8.81 (d, J=8.0 Hz, 1H), 8.35 (d, J=3.9 Hz, 1H), 8.21 (d, J=3.0 Hz, 1H), 7.55 (d, J=8.2 Hz, 1H), 7.30 (t, J=8.1 Hz, 1H), 7.21 (t, J=7.9 Hz, 1H), 7.06 (d, J=2.0 Hz, 1H), 6.90 (d, J=5.6 Hz, 2H), 3.92 (s, 3H), 3.05-2.94 (m, 2H), 2.78 (s, 2H), 2.56 (s, 3H), 2.51 (d, J=1.8 Hz, 1H), 2.45 (s, 6H).

### Procedure for the Synthesis of 263

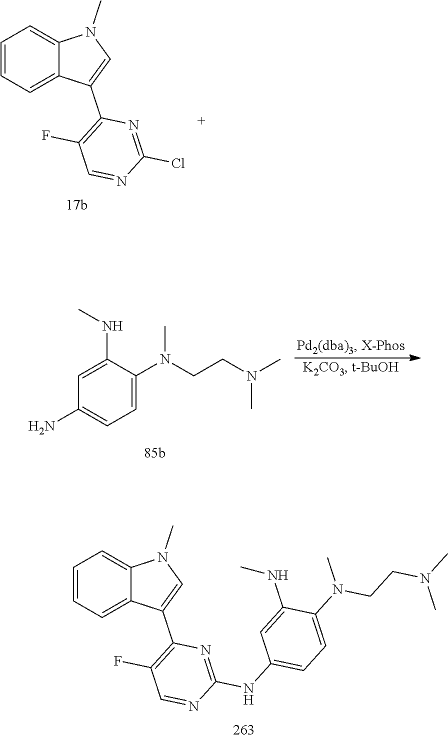

### N¹-(2-(dimethylamino)ethyl)-N⁴-(5-fluoro-4-(1-methyl-1H-indol-3-yl)pyrimidin-2-yl)-N¹,N²-dimethylbenzene-1,2,4-triamine (263)

Synthesis of 263 was similar to that of 259a. The residue was purified by flash column chromatography (dichloromethane/methanol=15/1) to give the title product 263 (13 mg, yield 3.6%) as a gray solid. LC-MS: m/z 448.4 [M+H]⁺. ¹H NMR (500 MHz, Chloroform-d) δ 9.61 (s, 1H), 9.47-9.40 (m, 1H), 8.95 (d, 1H), 8.81 (d, 1H), 8.80 (s, 1H), 8.20 (d, J=8.2 Hz, 1H), 7.99 (t, J=7.5 Hz, 1H), 7.87 (t, J=7.5 Hz, 1H), 7.64 (s, 2H), 7.56 (s, 1H), 3.43 (s, 3H), 3.29 (s, 3H), 3.23 (d, J=9.8 Hz, 4H), 3.01 (s, 6H), 1.93 (s, 3H).

### Procedure for the Synthesis of 264

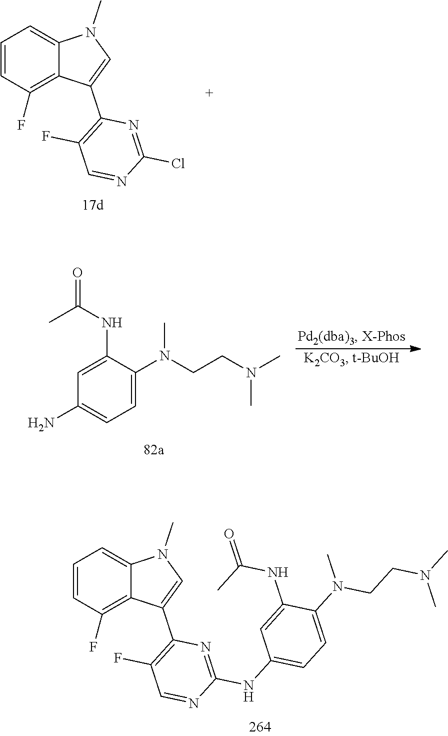

### N-(2-((2-(dimethylamino)ethyl)(methyl)amino)-5-((5-fluoro-4-(4-fluoro-1-methyl-1H-indol-3-yl)pyrimidin-2-yl)amino)phenyl)acetamide (264)

Synthesis of 264 was similar to that of 259a. The residue was purified by preparative thin layer chromatography (dichloromethane/methanol=12/1) to give the title product 264 (101 mg, yield 38.2%) as a gray solid MS-ESI (M+H)⁺: 494.4. ¹H NMR (500 MHz, DMSO-d₆) δ 9.74 (s, 1H), 9.51 (s, 1H), 8.64 (s, 1H), 8.48 (d, J=2.5 Hz, 1H), 8.13 (s, 1H), 7.47-7.42 (m, 1H), 7.40 (s, 1H), 7.26 (td, J=8.0, 4.8 Hz, 1H), 7.14 (d, J=8.8 Hz, 1H), 6.95 (dd, J=11.3, 7.8 Hz, 1H), 3.93 (s, 3H), 3.01 (s, 2H), 2.84 (s, 2H), 2.57 (s, 3H), 2.52 (s, 6H), 2.21 (s, 3H).

### Procedure for the Synthesis of 265

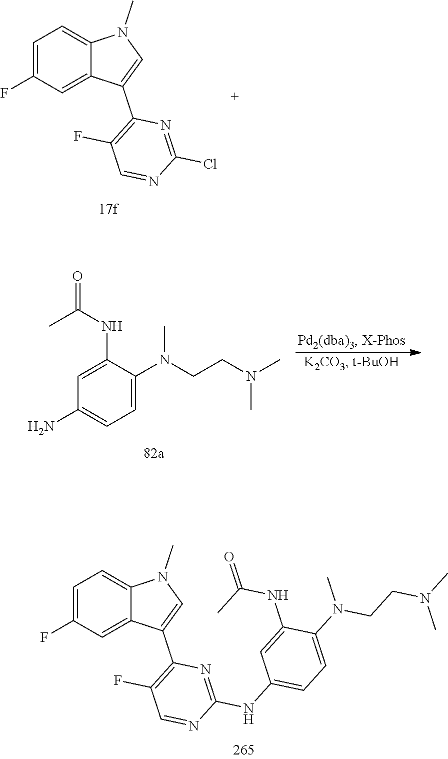

### N-(2-((2-(dimethylamino)ethyl)(methyl)amino)-5-((5-fluoro-4-(5-fluoro-1-methyl-1H-indol-3-yl)pyrimidin-2-yl)amino)phenyl)acetamide (265)

Synthesis of 265 was similar to that of 259a. The residue was purified by preparative thin layer chromatography (dichloromethane/methanol=12/1) to give the title product 265 (22 mg, yield 8.3%) as a gray solid LC-MS: m/z 494.4 [M+H]⁺. ¹H NMR (500 MHz, DMSO-d₆) δ 10.03 (s, 1H), 9.48 (s, 1H), 8.50 (d, J=10.0 Hz, 1H), 8.45 (d, J=2.6 Hz, 1H), 8.38 (d, J=3.8 Hz, 1H), 8.30 (d, J=2.8 Hz, 1H), 7.57 (dd, J=8.9, 4.5 Hz, 1H), 7.47 (dd, J=8.7, 2.6 Hz, 1H), 7.24 (d, J=8.7 Hz, 1H), 7.14 (td, J=9.1, 2.7 Hz, 1H), 3.92 (d, J=1.8 Hz, 4H), 2.81 (t, J=5.6 Hz, 2H), 2.66 (s, 3H), 2.29 (t, J=5.6 Hz, 2H), 2.23 (s, 6H), 2.06 (s, 3H).

### Procedure for the Synthesis of 266

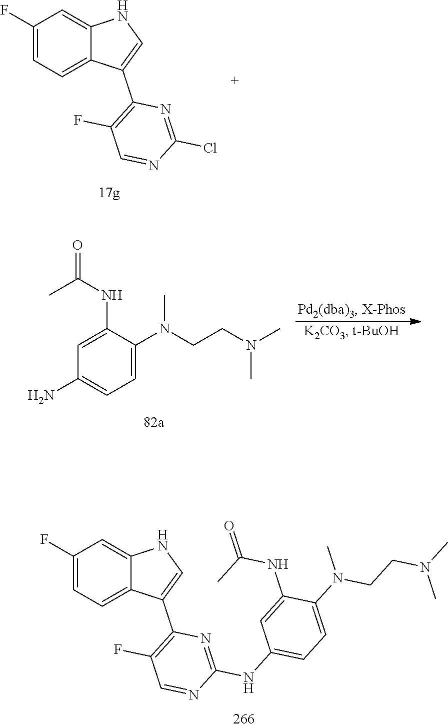

### N-(2-((2-(dimethylamino)ethyl)(methyl)amino)-5-((5-fluoro-4-(6-fluoro-1H-indol-3-yl)pyrimidin-2-yl)amino)phenyl)acetamide (266)

Synthesis of 266 was similar to that of 259a. The residue was purified by preparative thin layer chromatography (dichloromethane/methanol=12/1) to give the title product 266 (16 mg, yield 5.5%) as a gray solid. LC-MS: m/z 480.4 [M+H]⁺. ¹H NMR (500 MHz, DMSO-d₆) δ 12.06 (s, 1H), 9.74 (s, 1H), 9.48 (s, 1H), 8.79-8.71 (m, 1H), 8.39 (d, J=3.7 Hz, 1H), 8.37 (s, 1H), 8.19 (s, 1H), 7.52 (d, J=8.6 Hz, 1H), 7.30 (dd, J=9.6, 2.1 Hz, 1H), 7.21 (d, J=8.7 Hz, 1H), 7.05-6.98 (m, 1H), 3.08 (s, 2H), 2.93 (s, 2H), 2.60 (s, 3H), 2.57 (s, 6H), 2.18 (s, 3H).

### Procedure for the Synthesis of 267

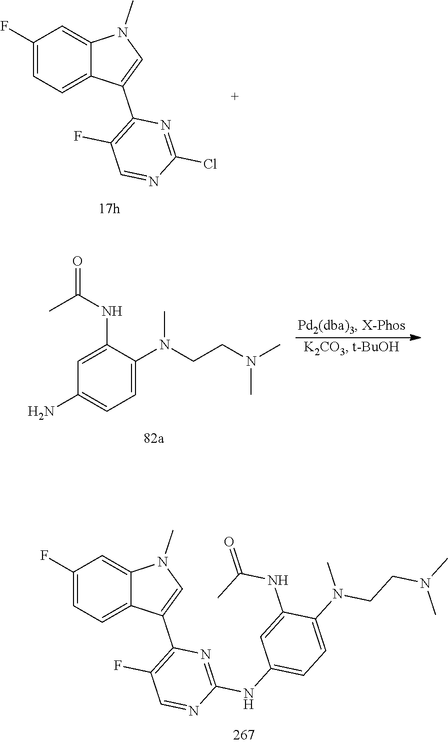

### N-(2-((2-(dimethylamino)ethyl)(methyl)amino)-5-((5-fluoro-4-(6-fluoro-1-methyl-1H-indol-3-yl)pyrimidin-2-yl)amino)phenyl)acetamide (267)

Synthesis of 267 was similar to that of 259a. The residue was purified by preparative thin layer chromatography (dichloromethane/methanol=12/1) to give the title product 267 (27 mg, yield 10.2%) as a gray solid MS-ESI (M+H)⁺: 494.4. ¹H NMR (500 MHz, DMSO-d₆) δ 9.73 (s, 1H), 9.49 (s, 1H), 8.82-8.73 (m, 1H), 8.40 (d, J=3.8 Hz, 1H), 8.38 (s, 1H), 8.25 (d, J=2.7 Hz, 1H), 7.54-7.48 (m, 1H), 7.45 (dd, J=9.9, 2.2 Hz, 1H), 7.22 (d, J=8.7 Hz, 1H), 7.04 (td, J=9.3, 2.1 Hz, 1H), 3.89 (s, 3H), 3.05 (s, 2H), 2.61 (s, 6H), 2.54 (s, 2H), 2.16 (s, 3H).

### Procedure for the Synthesis of 268

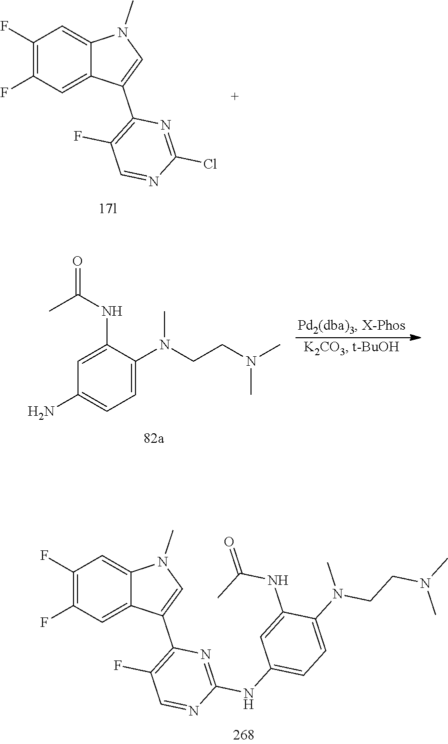

### N-(5-((4-(5,6-difluoro-1-methyl-1H-indol-3-yl)-5-fluoropyrimidin-2-yl)amino)-2-((2-(dimethylamino)ethyl)(methyl)amino)phenyl)acetamide (268)

Synthesis of 268 was similar to that of 259a. The residue was purified by flash column chromatography (dichloromethane/methanol=15/1) and preparative thin layer chromatography (dichloromethane/methanol=12/1) to give the title product 268 (10 mg, yield 2.8%) as a gray solid. LC-MS: m/z 512.5 [M+H]⁺.

### Procedure for the Synthesis of 269

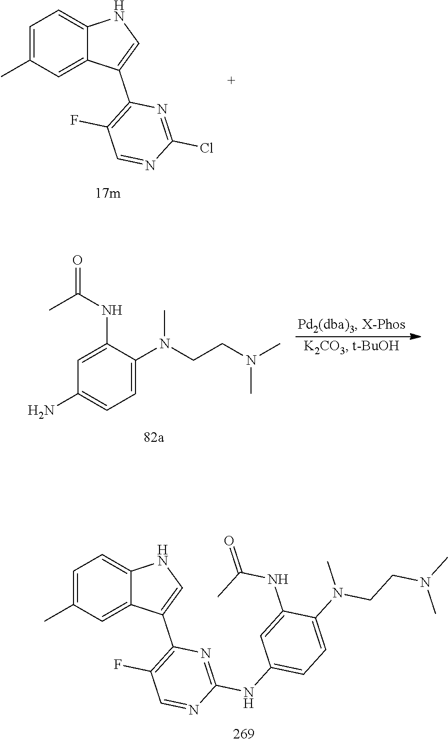

### N—N-(2-((2-(dimethylamino)ethyl)(methyl)amino)-5-((5-fluoro-4-(5-methyl-1H-indol-3-yl)pyrimidin-2-yl)amino)phenyl)acetamide (269)

Synthesis of 269 was similar to that of 259a. The residue was purified by flash column chromatography (dichloromethane/methanol=15/1) to give the title product 269 (26 mg, yield 7.2%) as a gray solid. LC-MS: m/z 476.2 [M+H]⁺. ¹H NMR (500 MHz, DMSO-d₆) δ 11.83 (d, J=3.0 Hz, 1H), 10.02 (s, 1H), 9.39 (s, 1H), 8.50 (s, 1H), 8.39-8.35 (m, 2H), 8.15-8.11 (m, 1H), 7.61 (dd, J=8.7, 2.6 Hz, 1H), 7.38 (d, J=8.3 Hz, 1H), 7.26 (d, J=8.7 Hz, 1H), 7.05 (dd, J=8.2, 1.7 Hz, 1H), 2.82 (t, J=5.6 Hz, 2H), 2.67 (s, 3H), 2.41 (s, 3H), 2.29 (t, J=5.7 Hz, 2H), 2.23 (s, 6H), 2.07 (s, 3H).

### Procedure for the Synthesis of 270

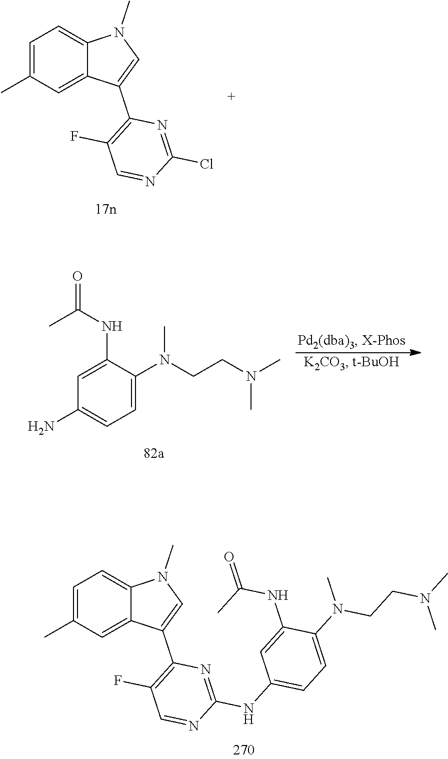

### N-(5-((4-(1,5-dimethyl-1H-indol-3-yl)-5-fluoropyrimidin-2-yl)amino)-2-((2-(dimethylamino)ethyl)(methyl)amino)phenyl)acetamide (270)

Synthesis of 270 was similar to that of 259a. The residue was purified by flash column chromatography (dichloromethane/methanol=15/1) to give the title product 270 (160 mg, yield 42.9%) as a gray solid. LC-MS: m/z 490.6 [M+H]⁺. ¹H NMR (500 MHz, DMSO-d₆) δ 10.02 (s, 1H), 9.39 (s, 1H), 8.50 (s, 1H), 8.38 (d, J=2.5 Hz, 1H), 8.36 (d, J=3.9 Hz, 1H), 8.19 (d, J=2.9 Hz, 1H), 7.57 (dd, J=8.7, 2.6 Hz, 1H), 7.43 (d, J=8.4 Hz, 1H), 7.26 (d, J=8.6 Hz, 1H), 7.11 (dd, J=8.4, 1.7 Hz, 1H), 3.89 (s, 3H), 2.81 (t, J=5.6 Hz, 2H), 2.67 (s, 3H), 2.42 (s, 3H), 2.28 (t, J=5.6 Hz, 2H), 2.23 (s, 6H), 2.06 (s, 3H).

### Procedure for the Synthesis of 271

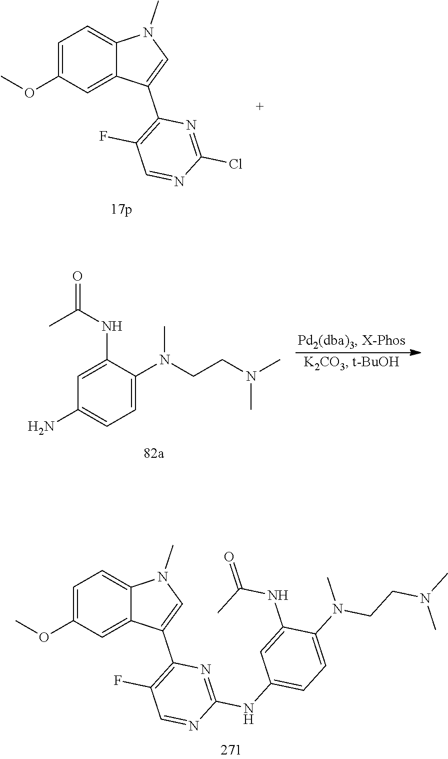

### N-(2-((2-(dimethylamino)ethyl)(methyl)amino)-5-((5-fluoro-4-(4-methoxy-1-methyl-1H-indol-3-yl)pyrimidin-2-yl)amino)phenyl)acetamide (271)

Synthesis of 271 was similar to that of 259a. The residue was purified by preparative thin layer chromatography (dichloromethane/methanol=12/1) to give the title product 271 (126 mg, yield 45.4%) as a gray solid MS-ESI (M+H)⁺: 506.3. ¹H NMR (500 MHz, DMSO-d₆) δ 9.83 (s, 1H), 9.42 (s, 1H), 8.36 (d, J=3.9 Hz, 1H), 8.34 (d, J=2.6 Hz, 1H), 8.19 (d, J=3.0 Hz, 1H), 8.17 (d, J=2.6 Hz, 1H), 7.56 (dd, J=8.7, 2.6 Hz, 1H), 7.45 (d, J=8.9 Hz, 1H), 7.20 (d, J=8.7 Hz, 1H), 6.94 (dd, J=8.9, 2.6 Hz, 1H), 3.89 (s, 3H), 3.75 (s, 3H), 2.93 (s, 2H), 2.62 (s, 3H), 2.41 (s, 6H), 2.12 (s, 3H).

### Procedure for the Syntheses of 272a and 272b

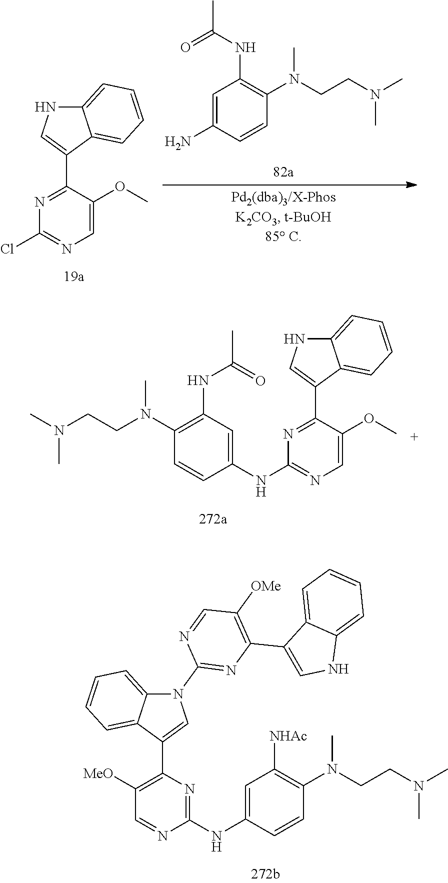

### N-(5-(4-(1H-indol-3-yl)-5-methoxypyrimidin-2-ylamino)-2-((2-(dimethylamino)ethyl)(methyl)amino)phenyl)acetamide (272a) & N-(5-(4-(1-(4-(1H-indol-3-yl)-5-methoxypyrimidin-2-yl)-1H-indol-3-yl)-5-methoxypyrimidin-2-ylamino)-2-((2-(dimethylamino)ethyl)(methyl)amino) phenyl)acetamide (272b)

To the mixture of intermediate 19a (0.270 g, 1.0 mmol) and intermediate 82a (0.250 g, 1.0 mmol) in t-BuOH (30 mL) were added K₂CO₃ (0.200 g, 1.5 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) and preparative thin layer chromatography to give 272a (20 mg, 4.2% yield, a brown solid) and 272b (15 mg, 2.2% yield, a brown solid). For 272a: MS-ESI (M+H)⁺: 474.5; For 272b: MS-ESI (M+H)⁺: 697.5

### Procedure for the Syntheses of 273a and 273b

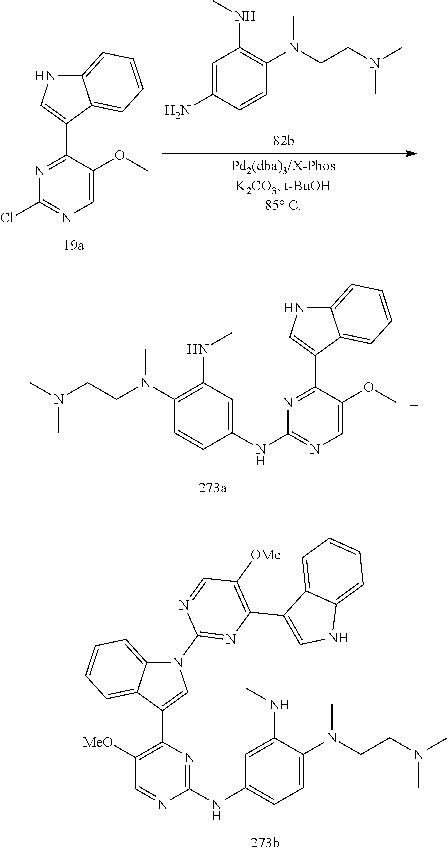

### N⁴-(4-(1H-indol-3-yl)-5-methoxypyrimidin-2-yl)-N1-(2-(dimethylamino)ethyl)-N¹,N²-dimethylbenzene-1,2,4-triamine (273a) & N⁴-(4-(1-(4-(1H-indol-3-yl)-5-methoxypyrimidin-2-yl)-1H-indol-3-yl)-5-methoxypyrimidin-2-yl)-N¹-(2-(dimethylamino)ethyl)-N¹,N²-dimethylbenzene-1,2,4-triamine (273b)

To the mixture of intermediate 19a (0.130 g, 0.5 mmol) and intermediate 85b (0.111 g, 0.5 mmol) in t-BuOH (30 mL) were added K₂CO₃ (0.200 g, 1.5 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) and preparative thin layer chromatography to give 273a (20 mg, 8.9% yield, a brown solid) and 273b (10 mg, 3.0% yield, a brown solid). For 273a: LC-MS: m/z 446.6 [M+H]⁺; For 273b: LC-MS: m/z 669.8 [M+H]⁺

### Procedure for the Synthesis of 274

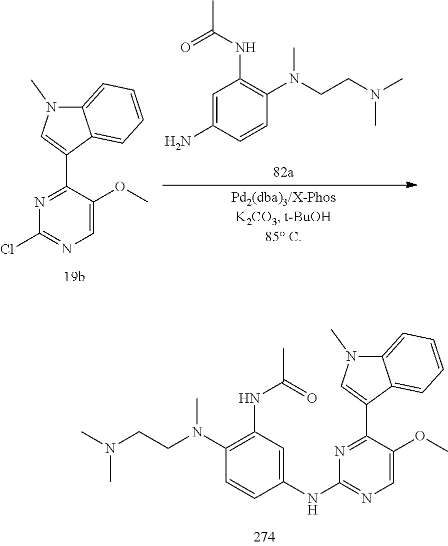

### N-(2-((2-(dimethylamino)ethyl)(methyl)amino)-5-(5-methoxy-4-(1-methyl-1H-indol-3-yl)pyrimidin-2-ylamino)phenyl)acetamide (274)

To the mixture of intermediate 19b (0.220 g, 0.8 mmol) and intermediate 82a (0.200 g, 0.8 mmol) in t-BuOH (20 mL) were added K₂CO₃ (0.150 g, 1.2 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) and preparative thin layer chromatography to give 274 (28 mg, 7% yield) as a brown solid. LC-MS: m/z 488.5 [M+H]⁺.

### Procedure for the Synthesis of 275

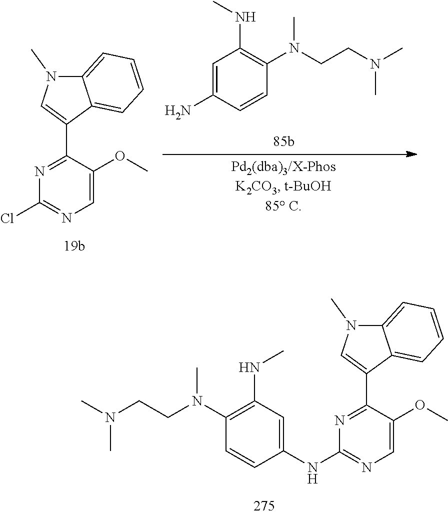

### N¹-(2-(dimethylamino)ethyl)-N⁴-(5-methoxy-4-(1-methyl-1H-indol-3-yl)pyrimidin-2-yl)-N¹,N²-dimethylbenzene-1,2,4-triamine (275)

To the mixture of intermediate 19b (0.140 g, 0.5 mmol) and intermediate 85b (0.111 g, 0.5 mmol) in t-BuOH (30 mL) were added K₂CO₃ (0.200 g, 1.5 mmol), Pd₂(dba)₃ (52 mg, 0.05 mmol) and X-Phos (51 mg, 0.1 mmol). The mixture was stirred at 85° C. overnight. After cooling to room temperature and concentration, the residue was purified by flash column chromatography (dichloromethane/methanol=30/1-20/1) and preparative thin layer chromatography to give 275 (10 mg, 4.3% yield) as a brown solid. LC-MS: m/z 460.7 [M+H]⁺.

### Procedure for the Synthesis of 276

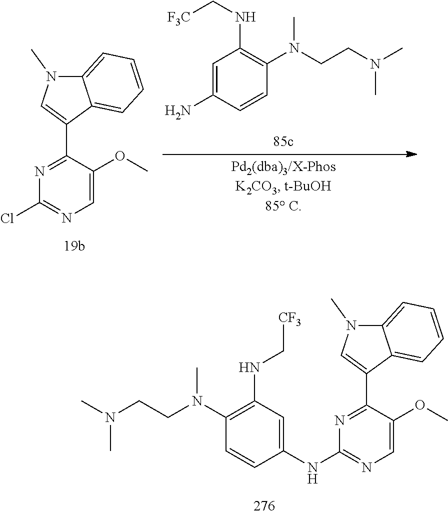

### N¹-(2-(dimethylamino)ethyl)-N⁴-(5-methoxy-4-(1-methyl-1H-indol-3-yl)pyrimidin-2-yl)-N¹-methyl-N²-(2,2,2-trifluoroethyl)benzene-1,2,4-triamine (276)

To the mixture of intermediate 19b (0.200 g, 0.8 mmol) and intermediate 85c (0.120 g, 0.4 mmol) in t-BuOH (20 mL) were added K₂CO₃ (0.150 g, 1.2 mmol), Pd₂(dba)₃ (50 mg, 0.05 mmol) and X-Phos (50 mg, 0.1 mmol). The mixture was stirred at 85° C. overnight. After cooling to room temperature and concentration, the residue was purified by flash column chromatography (dichloromethane/methanol=30/1-20/1) and preparative thin layer chromatography to give 276 (15 mg, 6.9% yield) as a brown solid. MS-ESI (M+H)⁺: 528.3.

### Procedure for the Synthesis of 277

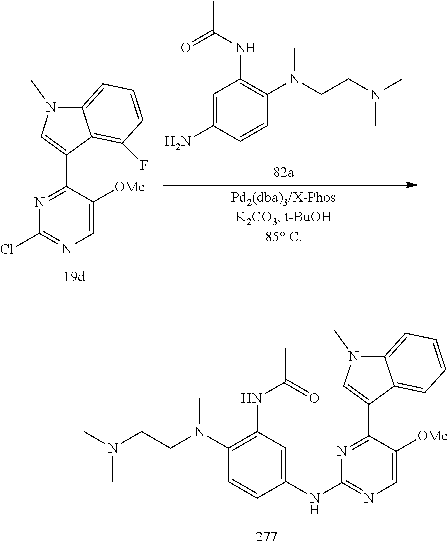 [SMILES: COc1cnc(Cl)nc1-c1cn(C)c2cccc(F)c12 ; CC(=O)Nc1cc(N)ccc1N(C)CCN(C)C ; COc1cnc(Nc2ccc(N(C)CCN(C)C)c(NC(C)=O)c2)nc1-c1cn(C)c2ccccc12]

### N-(2-((2-(dimethylamino)ethyl)(methyl)amino)-5-((4-(4-fluoro-1-methyl-1H-indol-3-yl)-5-methoxypyrimidin-2-yl)amino)phenyl)acetamide (277)

A mixture of intermediate 19d (420 mg, 1.44 mmol), intermediate 82a (199.1 mg, 0.80 mmol), Pd₂(dba)₃ (152.8 mg, 0.17 mmol), X-Phos (144.1 mg, 0.34 mmol) and K₂CO₃ (606.5 mg, 4.39 mmol) in t-BuOH (30 mL) was stirred under N₂ at refluxing temperature for 15.5 hrs. After cooling to room temperature, the reaction mixtures was filtered through Celite® and the filtrates were concentrated under reduced pressure to dryness. The crude was purified by flash column chromatography (dichloromethane/methanol=20/1) to give the title compound 277 (30 mg, yield 7.5%) as a brown solid.

### Procedure for the Synthesis of 278: N-(2-acetamido-4-((4-(7-fluoro-1H-indol-3-yl)-5-methoxypyrimidin-2-yl)amino)phenyl)-N-(2-(dimethylamino)ethyl)acetamide (278)

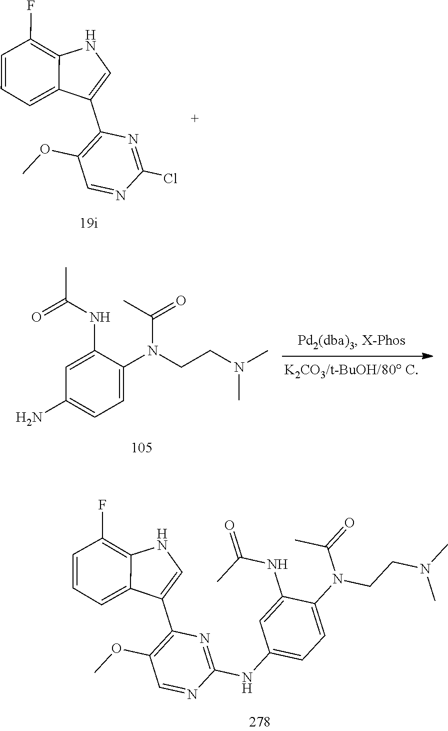 [SMILES: COc1cnc(Cl)nc1-c1c[nH]c2c(F)cccc12 ; CC(=O)Nc1cc(N)ccc1N(CCN(C)C)C(C)=O ; COc1cnc(Nc2ccc(N(CCN(C)C)C(C)=O)c(NC(C)=O)c2)nc1-c1c[nH]c2c(F)cccc12]

Synthesis of 278 was similar to that of 272a. The residue was purified by preparative thin layer chromatography (dichloromethane/methanol=12/1) to give the title product 278 (64 mg, yield 26.2%) as a yellow solid. MS-ESI (M+Na)⁺: 542.6. ¹H NMR (500 MHz, Chloroform-d) δ 11.23 (s, 1H), 10.02 (s, 1H), 8.48 (d, J=8.1 Hz, 1H), 8.43 (d, J=2.1 Hz, 1H), 8.22 (d, J=2.7 Hz, 1H), 7.95 (s, 1H), 7.54 (dd, J=8.6, 2.3 Hz, 1H), 7.17 (s, 1H), 7.11 (dd, J=7.9, 5.1 Hz, 1H), 7.08 (d, J=8.6 Hz, 1H), 6.95 (dd, J=10.8, 7.9 Hz, 1H), 4.96 (dt, J=14.2, 6.9 Hz, 1H), 3.86 (s, 3H), 2.94 (d, J=14.6 Hz, 1H), 2.46 (s, 2H), 2.34 (s, 6H), 2.17 (s, 3H), 1.83 (s, 3H).

### Procedure for the Synthesis of 279

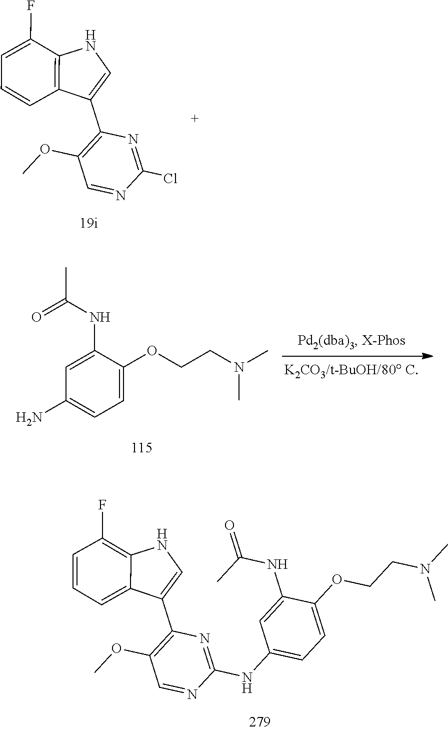 [SMILES: COc1cnc(Cl)nc1-c1c[nH]c2c(F)cccc12 ; CC(=O)Nc1cc(N)ccc1OCCN(C)C ; COc1cnc(Nc2ccc(OCCN(C)C)c(NC(C)=O)c2)nc1-c1c[nH]c2c(F)cccc12]

### N-(2-(2-(dimethylamino)ethoxy)-5-((4-(7-fluoro-1H-indol-3-yl)-5-methoxypyrimidin-2-yl)amino)phenyl)acetamide (279)

A mixture of 19i (155 mg, 0.56 mmol), 115 (129 mg, 0.54 mmol), X-Phos (65 mg, 0.13 mmol), K₂CO₃ (237 g, 1.72 mmol), and Pd₂(dba)₃ (63 mg, 0.068 mmol) in t-BuOH (10 mL) was stirred at 80° C. overnight. The reaction was cooled to room temperature and then filtered. The filtrate was concentrated and purified by preparative thin layer chromatography to give 279 (7.6 mg, ˜3% yield). MS-ESI (M+H⁺): 479.2; ¹H NMR (500 MHz, DMSO-d₆) δ 12.32 (s, 1H), 9.68 (s, 1H), 9.07 (s, 1H), 8.67 (d, J=8.0 Hz, 1H), 8.35 (d, J=2.9 Hz, 1H), 8.33-8.27 (m, 1H), 8.23 (s, 1H), 7.51 (m, 1H), 7.13-6.96 (m, 3H), 4.15 (m, 2H), 3.97 (s, 3H), 3.01-2.76 (m, 2H), 2.46 (s, 6H), 2.10 (s, 3H).

### Procedure for the Synthesis of R-280: (R)—N-(2-(3-(dimethylamino)pyrrolidin-1-yl)-5-((4-(7-fluoro-1H-indol-3-yl)-5-methoxypyrimidin-2-yl)amino)phenyl)acetamide (R-280)

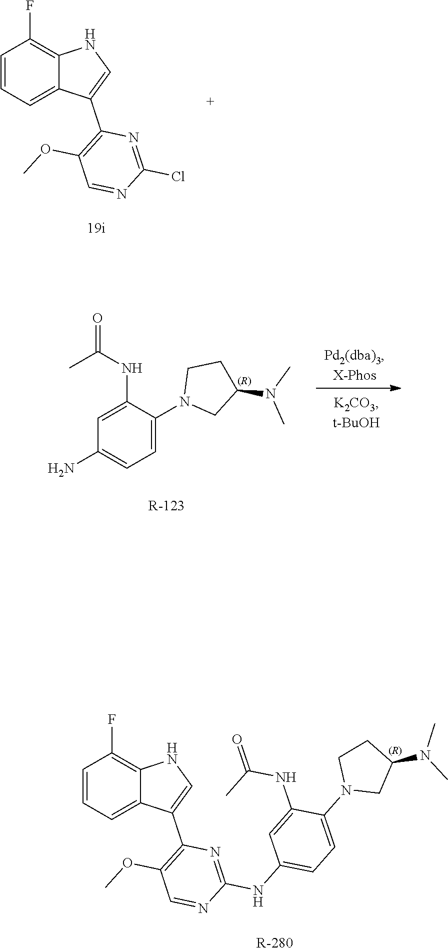

Synthesis of R-280 was similar to that of 272a. The residue was purified by preparative thin layer chromatography (dichloromethane/methanol=12/1) to give the title product R-280 (35 mg, yield 18.5%) as a brown solid. MS-ESI (M+H)⁺: 504.6. ¹H NMR (500 MHz, DMSO-d₆) δ 12.25 (s, 1H), 9.11 (s, 1H), 9.07 (s, 1H), 8.68 (d, J=8.0 Hz, 1H), 8.36 (s, 1H), 8.24 (s, 1H), 8.02 (s, 1H), 7.54 (d, J=8.2 Hz, 1H), 7.11 (td, J=7.9, 5.3 Hz, 1H), 7.05 (dd, J=11.1, 7.9 Hz, 1H), 6.98 (d, J=8.5 Hz, 1H), 3.97 (s, 3H), 3.22-3.03 (m, 4H), 2.59 (s, 5H), 2.22 (s, 1H), 2.10 (s, 3H), 2.04-1.96 (m, 1H).

### Procedure for the Synthesis of S-280: (S)—N-(2-(3-(dimethylamino)pyrrolidin-1-yl)-5-((4-(7-fluoro-1H-indol-3-yl)-5-methoxypyrimidin-2-yl)amino)phenyl)acetamide (S-280)

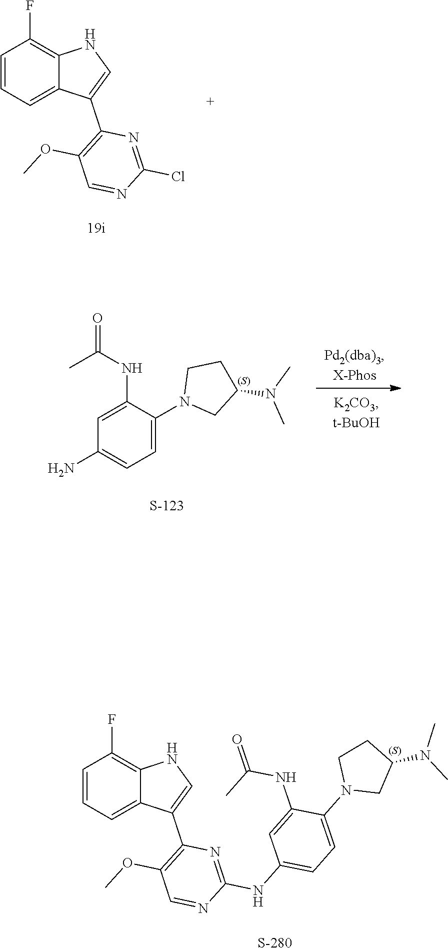

Synthesis of S-280 was similar to that of R-280. The residue was purified by preparative thin layer chromatography (dichloromethane/methanol=12/1) to give the title product S-280 (47 mg, yield 24.7%) as a brown solid. MS-ESI (M+H)⁺: 504.5. ¹H NMR (500 MHz, DMSO-d₆) δ 12.24 (s, 1H), 9.15 (s, 1H), 9.07 (s, 1H), 8.67 (d, J=8.2 Hz, 1H), 8.36 (s, 1H), 8.24 (s, 1H), 8.09 (s, 1H), 7.55 (d, J=8.5 Hz, 1H), 7.11 (q, J=7.8 Hz, 1H), 7.08-6.98 (m, 2H), 3.98 (s, 3H), 3.18 (s, 2H), 2.99 (s, 1H), 2.71 (s, 6H), 2.27 (s, 1H), 2.14 (s, 3H), 2.07-1.94 (m, 1H).

### Procedure for the Synthesis of R-281: (R)—N-(2-(3-(dimethylamino)pyrrolidin-1-yl)-5-((4-(7-fluoro-1-methyl-1H-indol-3-yl)-5-methoxypyrimidin-2-yl)amino)phenyl)acetamide (R-281)

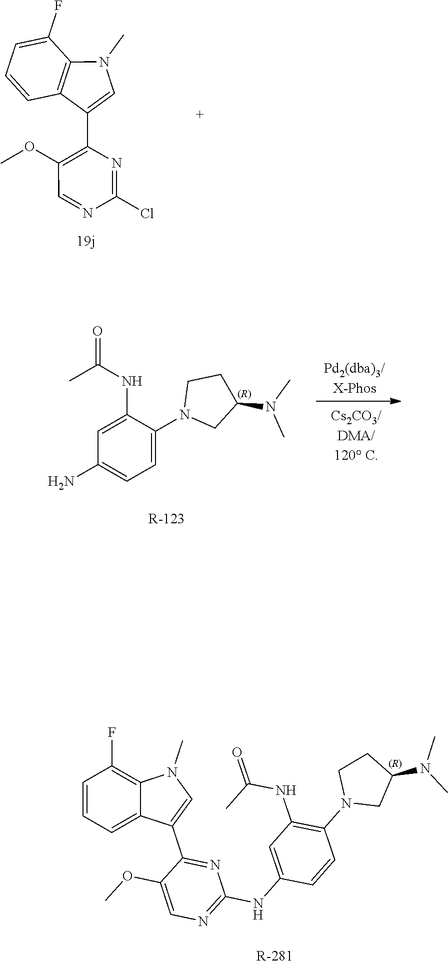

A mixture of 19j (200 mg, 0.68 mmol), X-Phos (65 mg, 0.13 mmol), Cs₂CO₃ (477 g, 1.72 mmol), R-123 (180 mg, 0.68 mmol) and Pd₂(dba)₃ (63 mg, 0.068 mmol) in DMA (10 mL) was stirred at 120° C. overnight. After cooling to room temperature, the mixture was filtered. The filtrate was concentrated and purified by preparative thin layer chromatography (dichloromethane/methanol=10/1) to give R-281 as an off-white solid (10 mg, ˜2.8% yield). MS-ESI (M+H⁺): 518.4; ¹H NMR (500 MHz, DMSO-d₆) δ 9.09 (s, 1H), 8.98 (s, 1H), 8.68 (d, J=8.0 Hz, 1H), 8.34 (s, 1H), 8.22 (s, 1H), 7.88 (s, 1H), 7.48 (d, J=8.2 Hz, 1H), 7.15-7.07 (m, 1H), 7.03 (m, 1H), 6.89 (d, J=8.6 Hz, 1H), 4.07 (s, 3H), 3.96 (s, 3H), 3.27-3.20 (m, 2H), 3.17 (s, 3H), 3.12 (m, 2H), 3.09-3.01 (m, 1H), 2.22 (s, 6H), 2.11-2.06 (m, 1H), 1.85-1.69 (m, 1H)

### Procedure for the Synthesis of 283

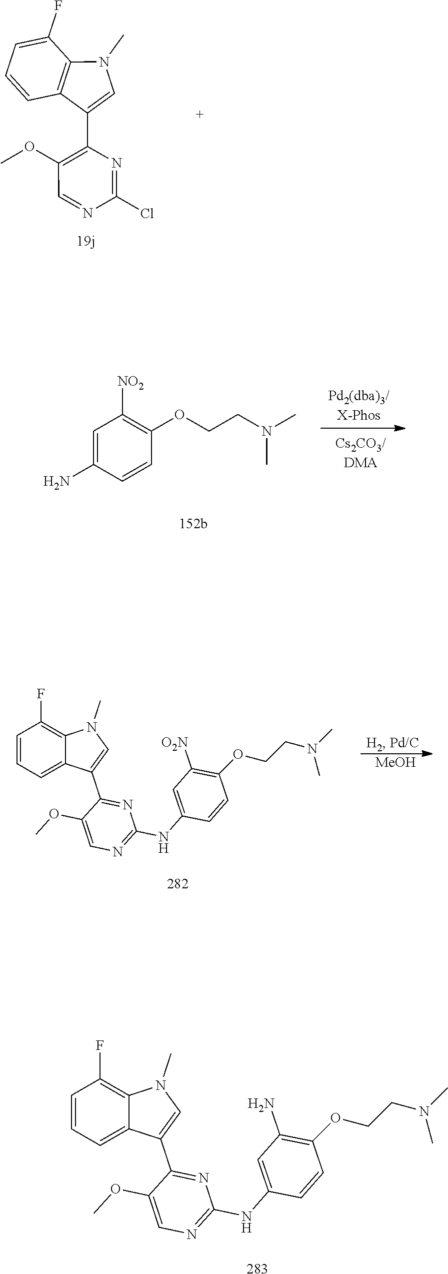

### N-(4-(2-(dimethylamino)ethoxy)-3-nitrophenyl)-4-(7-fluoro-1-methyl-1H-indol-3-yl)-5-methoxypyrimidin-2-amine (282)

A mixture of 19j (300 mg, 1.0 mmol), X-Phos (100 mg, 0.2 mmol), Cs₂CO₃ (677 g, 2.5 mmol), 152b (233 mg, 1.0 mmol) and Pd₂(dba)₃ (94 mg, 0.1 mmol) in DMA (10 mL) was stirred at 120° C. overnight. After cooling to room temperature, the mixture was filtered. The filtrate was concentrated and purified by flash column chromatography (dichloromethane/methanol=10/1) to give 282 as a yellow solid (400 mg, ˜83% yield). MS-ESI (M+H⁺): 481.5

### 4-(2-(dimethylamino)ethoxy)-N¹-(4-(7-fluoro-1-methyl-1H-indol-3-yl)-5-methoxypyrimidin-2-yl)benzene-1,3-diamine (283)

A mixture of 282 (400 mg, 0.83 mmol), Pd/C (5%, 40 mg) in MeOH (50 mL) and was stirred at 50° C. overnight under an atmosphere of hydrogen (balloon). The reaction mixture was filtered and the filtrate was then concentrated under vacuum to provide 283 (110 mg, 28.9% yield) as off-white solid by preparative thin layer chromatography. MS-ESI (M+H⁺): 451.2; ¹H NMR (500 MHz, DMSO-d₆) δ 8.74 (s, 1H), 8.70 (d, J=8.0 Hz, 1H), 8.33 (s, 1H), 8.19 (s, 1H), 7.27-6.96 (m, 3H), 6.93-6.84 (m, 1H), 6.74 (d, J=8.5 Hz, 1H), 4.05 (m, 5H), 3.95 (s, 3H), 2.90 (s, 2H), 2.50 (m, 2H), 2.44 (s, 6H).

### Procedure for the Synthesis of 284: N-(2-(2-(dimethylamino)ethoxy)-5-((5-methoxy-4-(7-methyl-1H-indol-3-yl)pyrimidin-2-yl)amino)phenyl)acetamide (284)

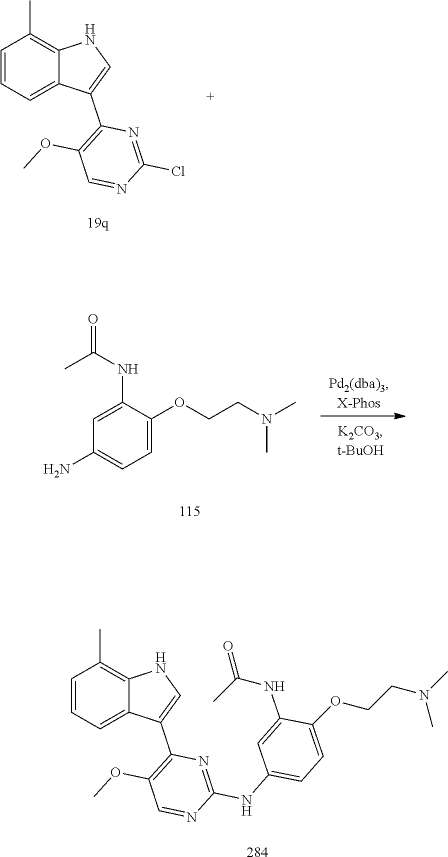 [SMILES: COc1cnc(Cl)nc1-c1c[nH]c2c(C)cccc12 ; CC(=O)Nc1cc(N)ccc1OCCN(C)C ; "115" ; COc1cnc(Nc2ccc(OCCN(C)C)c(NC(C)=O)c2)nc1-c1c[nH]c2c(C)cccc12 ; "284"]

A mixture of 19q (0.15 g, 0.55 mmol), 115 (0.13 g, 0.55 mmol), Pd₂(dba)₃ (0.10 g, w %=66%), X-Phos (0.11 g, w %=73%) and K₂CO₃ (0.30 g 2.17 mmol) in t-BuOH (15 mL) was stirred at 90° C. overnight. After cooling to 45° C., the mixture was filtered off and the filtrate was concentrated under reduced pressure. The residue was dissolved in ethyl acetate (40 mL), and washed with H₂O (20 mL*2), dried with Na₂SO₄. After concentration, the residue was purified by preparative thin layer chromatography (dichloromethane/methanol=12/1) to give the desired product 284 (20 mg, yield 7.7%) as a dark yellow solid. MS-ESI (M+H)⁺: 475.5. ¹H NMR (500 MHz, DMSO-d₆) δ 11.70 (s, 1H), 9.62 (s, 1H), 9.03 (s, 1H), 8.69 (d, J=7.9 Hz, 1H), 8.31 (d, J=3.0 Hz, 2H), 8.19 (s, 1H), 7.53 (dd, J=8.8, 2.5 Hz, 1H), 7.07-6.97 (m, 3H), 4.15 (t, J=5.1 Hz, 2H), 3.97 (s, 3H), 2.86 (s, 2H), 2.52 (s, 3H), 2.46 (s, 6H), 2.09 (s, 3H).

### Procedure for the Synthesis of 285: N-(5-((4-(1,7-dimethyl-1H-indol-3-yl)-5-methoxypyrimidin-2-yl) amino)-2-(2-(dimethylamino)ethoxy)phenyl)acetamide (285)

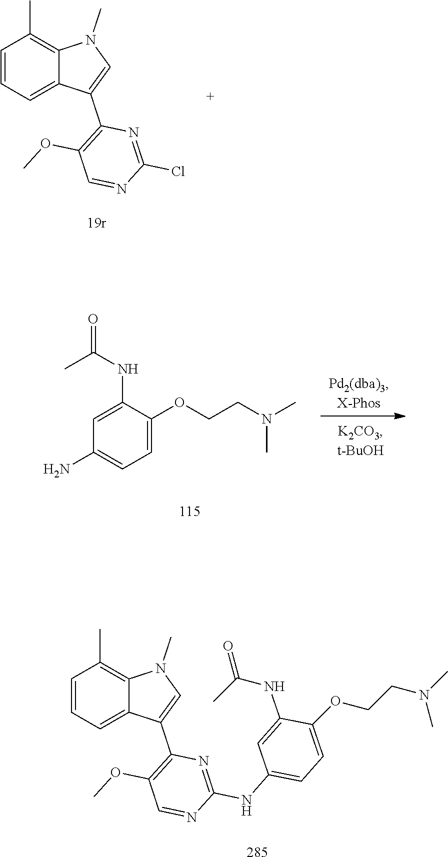 [SMILES: COc1cnc(Cl)nc1-c1cn(C)c2c(C)cccc12 ; CC(=O)Nc1cc(N)ccc1OCCN(C)C ; "115" ; COc1cnc(Nc2ccc(OCCN(C)C)c(NC(C)=O)c2)nc1-c1cn(C)c2c(C)cccc12 ; "285"]

Synthesis of 285 was similar to that of 284. The residue was purified by preparative thin layer chromatography (dichloromethane/methanol=12/1) to give the title product 285 (28 mg, yield 11.5%) as a yellow solid. MS-ESI (M+H)⁺: 489.7. ¹H NMR (500 MHz, DMSO-d₆) δ 9.62 (s, 1H), 8.99 (s, 1H), 8.75 (d, J=8.0 Hz, 1H), 8.31 (s, 1H), 8.27 (s, 1H), 8.18 (s, 1H), 7.50 (dd, J=8.8, 2.3 Hz, 1H), 7.05-6.98 (m, 2H), 6.94 (d, J=7.0 Hz, 1H), 4.16 (s, 3H), 3.95 (s, 3H), 2.77 (s, 4H), 2.40 (s, 6H), 2.08 (s, 3H).

### Procedure for the Synthesis of 286: N-(2-acetamido-4-((5-methoxy-4-(7-methoxy-1H-indol-3-yl) pyrimidin-2-yl)amino)phenyl)-N-(2-(dimethylamino)ethyl)acetamide (286)

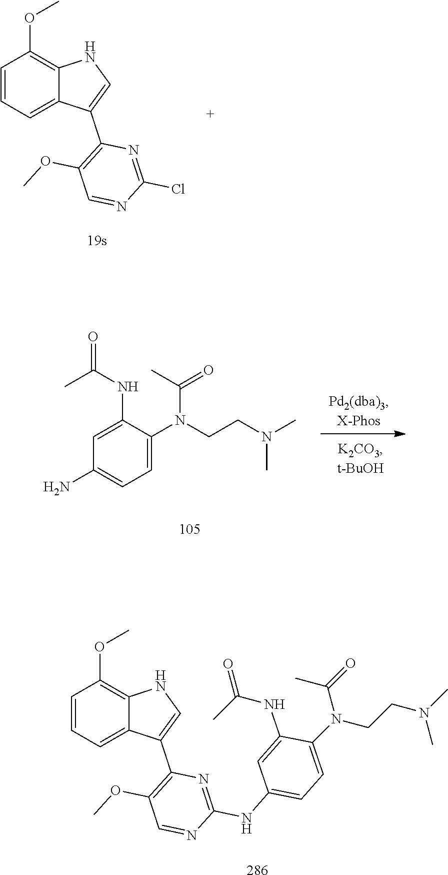 [SMILES: COc1cnc(Cl)nc1-c1c[nH]c2c(OC)cccc12 ; CC(=O)Nc1cc(N)ccc1N(CCN(C)C)C(C)=O ; "105" ; COc1cnc(Nc2ccc(N(CCN(C)C)C(C)=O)c(NC(C)=O)c2)nc1-c1c[nH]c2c(OC)cccc12 ; "286"]

A mixture of 19s (0.20 g, 0.69 mmol), 105 (0.10 g, 0.36 mmol), Pd₂(dba)₃ (0.11 g, w %=110%), X-Phos (0.11 g, w %=110%) and K₂CO₃ (0.31 g 2.25 mmol) in t-BuOH (15 mL) was stirred at 100° C. overnight. After cooling to 45° C., the mixture was filtered off and the filtrate was concentrated under reduced pressure. The residue was dissolved in DCM (40 mL), and washed with H₂O (20 mL*2), dried with Na₂SO₄. After concentration, the residue was purified by preparative thin layer chromatography (dichloromethane/methanol=12/1) to give the desired product 286 (8 mg, yield 4.4%) as a light brown solid. MS-ESI (M+H)⁺: 532.4. ¹H NMR (500 MHz, Chloroform-d) δ 11.05 (s, 1H), 8.89 (s, 1H), 8.34 (d, J=8.2 Hz, 1H), 8.29 (s, 1H), 8.25 (s, 1H), 8.12 (s, 1H), 7.82 (d, J=8.3 Hz, 1H), 7.23 (s, 1H), 7.17 (t, J=7.8 Hz, 1H), 7.08 (d, J=8.5 Hz, 1H), 6.75 (d, J=7.6 Hz, 1H), 5.01-4.89 (m, 1H), 3.99 (s, 3H), 3.97 (s, 3H), 2.93 (d, J=14.3 Hz, 1H), 2.45 (s, 2H), 2.33 (s, 6H), 2.12 (s, 3H), 1.79 (s, 3H).

### Procedure for the Synthesis of 287

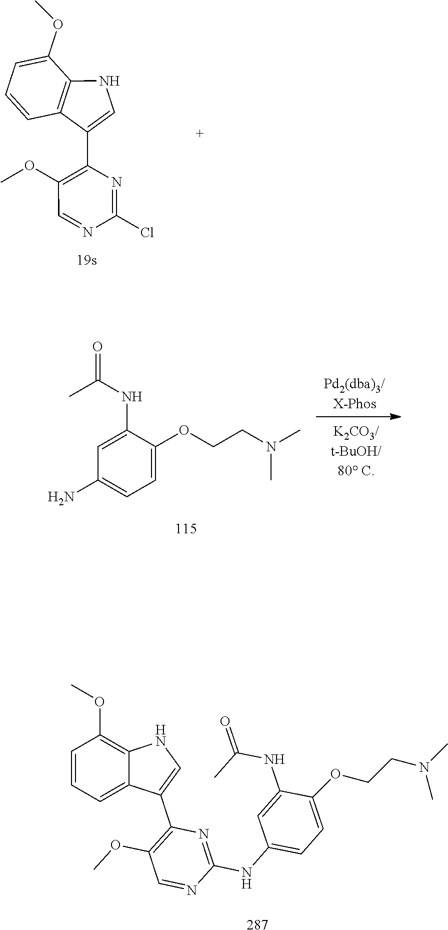 [SMILES: COc1cnc(Cl)nc1-c1c[nH]c2c(OC)cccc12 ; CC(=O)Nc1cc(N)ccc1OCCN(C)C ; "115" ; COc1cnc(Nc2ccc(OCCN(C)C)c(NC(C)=O)c2)nc1-c1c[nH]c2c(OC)cccc12 ; "287"]

### N-(2-(2-(dimethylamino)ethoxy)-5-((5-methoxy-4-(7-methoxy-1H-indol-3-yl)pyrimidin-2-yl)amino)phenyl)acetamide (287)

A mixture of 19s (159 mg, 0.55 mmol), 115 (115 mg, 0.48 mmol), X-Phos (65 mg, 0.13 mmol), K₂CO₃ (237 g, 1.72 mmol), and Pd₂(dba)₃ (63 mg, 0.068 mmol) in t-BuOH (10 mL) was stirred at 80° C. overnight. The reaction mixture was then cooled to room temperature and filtered. The filtrate was concentrated and purified by preparative thin layer chromatography to give 287 (22.7 mg, ˜9.64% yield). MS-ESI (M+H⁺): 491.4; ¹H NMR (500 MHz, DMSO-d₆) δ 9.64 (s, 1H), 8.96 (s, 1H), 8.41 (d, J=8.1 Hz, 1H), 8.33-8.26 (m, 1H), 8.23 (s, 1H), 8.13 (s, 1H), 7.52 (dd, J=8.8, 2.5 Hz, 1H), 7.00 (m, 2H), 6.73 (d, J=7.7 Hz, 1H), 5.75 (s, 1H), 4.07 (t, J=5.5 Hz, 2H), 3.94 (s, 6H), 2.55 (t, J=5.5 Hz, 2H), 2.28 (s, 6H), 2.05 (s, 3H).

### Procedure for the Synthesis of R-288

### (R)—N-(2-(3-(dimethylamino)pyrrolidin-1-yl)-5-((5-methoxy-4-(7-methoxy-1H-indol-3-yl)pyrimidin-2-yl)amino)phenyl)acetamide (R-288)

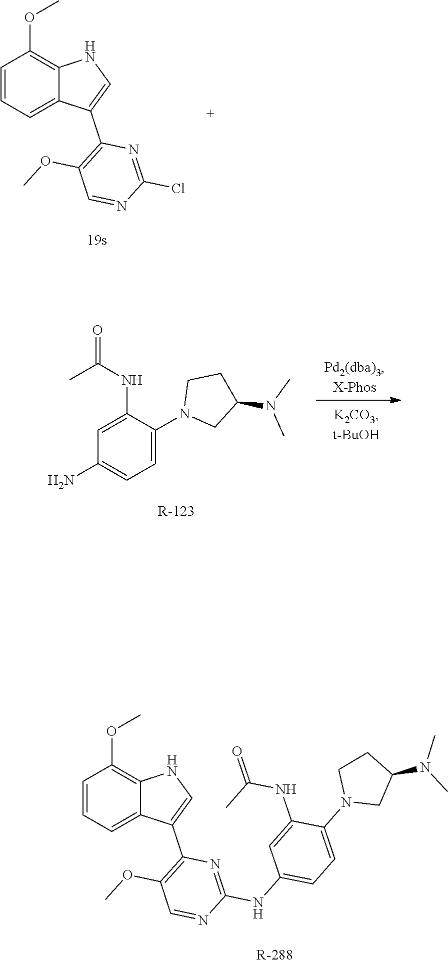 [SMILES: COc1cnc(Cl)nc1-c1c[nH]c2c(OC)cccc12 ; CC(=O)Nc1cc(N)ccc1N1CC[C@@H](N(C)C)C1 ; "R-123" ; COc1cnc(Nc2ccc(N3CC[C@@H](N(C)C)C3)c(NC(C)=O)c2)nc1-c1c[nH]c2c(OC)cccc12 ; "R-288"]

A mixture of 19s (0.12 g, 0.41 mmol), R-123 (0.10 g, 0.38 mmol), Pd₂(dba)₃ (0.10 g, w %=100%), X-Phos (0.15 g, w %=150%) and K₂CO₃ (0.30 g 2.17 mmol) in t-BuOH (15 mL) was stirred at 90° C. overnight. After cooling to 45° C., the mixture was filtered off and the filtrate was concentrated under reduced pressure. The residue was dissolved in ethyl acetate (50 mL), and washed with H₂O (20 mL*2), dried with Na₂SO₄. After concentration, the residue was purified by preparative thin layer chromatography (dichloromethane/methanol=12/1) to give the desired product R-288 (13 mg, yield 6.7%) as a yellow solid. MS-ESI (M+H)⁺: 516.6. ¹H NMR (500 MHz, DMSO-d₆) δ 11.85 (s, 1H), 9.10 (s, 1H), 9.01 (s, 1H), 8.44 (d, J=8.1 Hz, 1H), 8.22 (d, J=2.9 Hz, 1H), 8.19 (s, 1H), 8.01 (s, 1H), 7.56 (d, J=8.5 Hz, 1H), 7.07 (t, J=7.9 Hz, 1H), 6.97 (d, J=8.7 Hz, 1H), 6.78 (d, J=7.8 Hz, 1H), 3.96 (s, 5H), 3.14 (dd, J=21.6, 12.3 Hz, 4H), 2.55 (s, 6H), 2.26-2.16 (m, 1H), 2.10 (s, 3H), 2.00 (dq, J=12.8, 7.1, 6.1 Hz, 2H).

### Procedure for the Synthesis of S-288

### (S)—N-(2-(3-(dimethylamino)pyrrolidin-1-yl)-5-((5-methoxy-4-(7-methoxy-1H-indol-3-yl)pyrimidin-2-yl)amino)phenyl)acetamide (S-288)

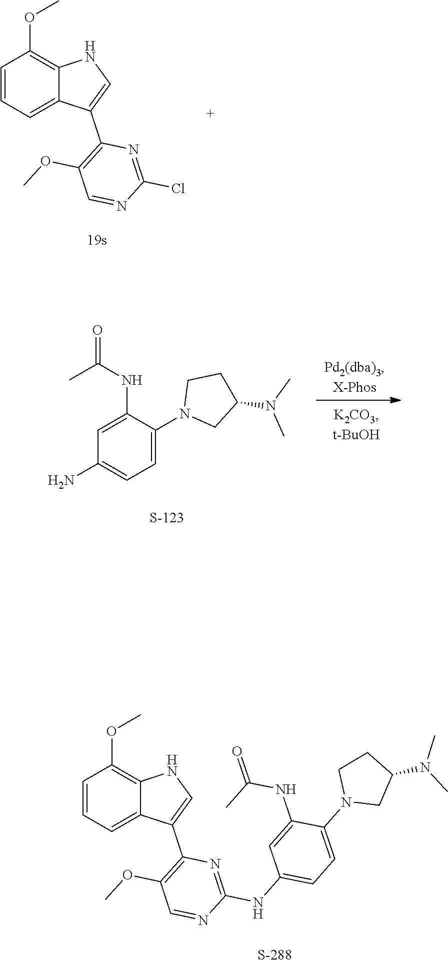

Synthesis of S-288 was similar to that of R-288. The residue was purified by preparative thin layer chromatography (dichloromethane/methanol=12/1) to give the title product S-288 (16 mg, yield 8.0%) as a brown solid. MS-ESI (M+H)⁺: 516.6. ¹H NMR (500 MHz, DMSO-d₆) δ 11.85 (s, 1H), 9.12 (s, 1H), 9.02 (s, 1H), 8.44 (d, J=8.1 Hz, 1H), 8.23 (d, J=3.0 Hz, 1H), 8.19 (s, 1H), 8.04 (s, 1H), 7.56 (dd, J=8.7, 2.2 Hz, 1H), 7.07 (t, J=7.9 Hz, 1H), 6.98 (d, J=8.7 Hz, 1H), 6.78 (d, J=7.7 Hz, 1H), 3.96 (s, 6H), 3.25-3.02 (m, 4H), 2.62 (s, 5H), 2.31-2.21 (m, 1H), 2.12 (s, 3H), 2.02-1.96 (m, 1H).

### Procedure for the Synthesis of 289

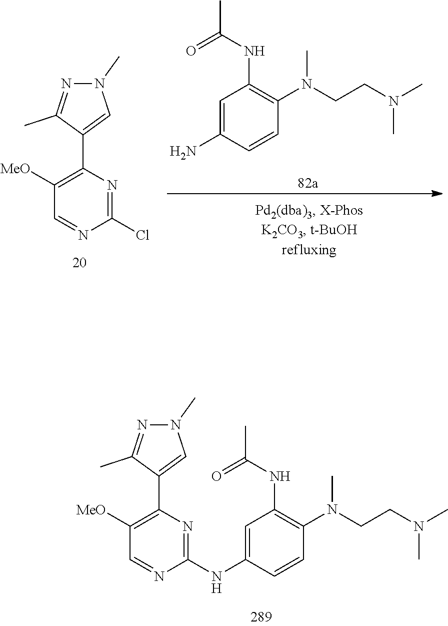

### N-(5-((4-(1,3-dimethyl-1H-pyrazol-4-yl)-5-methoxypyrimidin-2-yl)amino)-2-((2-(dimethylamino)ethyl)(methyl)amino)phenyl)acetamide (289)

A mixture of intermediate 20 (0.191 g, 0.8 mmol), intermediate 82a (0.20 g, 0.8 mmol), Pd2(dba)3 (0.1 g, 0.11 mmol), X-Phos (0.1 g, 0.21 mmol) and K₂CO₃ (0.58 g, 4.2 mmol) in t-BuOH (10 mL) was stirred under N₂ at refluxing temperature for 72 hrs. After cooling to room temperature, the reaction mixtures was filtered through Celite® and the filtrates were concentrated under reduced pressure to dryness. The crude was purified by preparative thin layer chromatography to give the title compound 289 (23.8 mg, yield 6.6%) as a light-yellow solid.

### Procedure for the Synthesis of 290

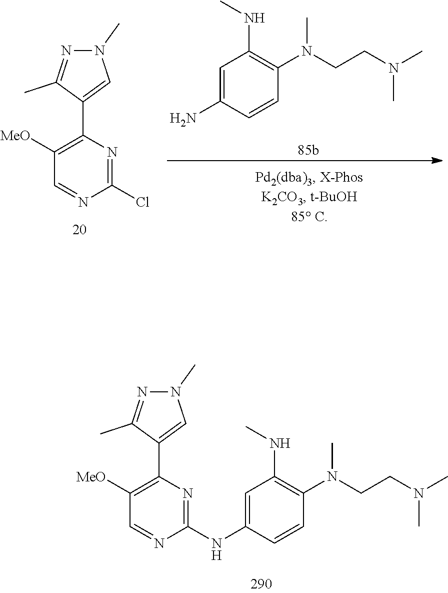

### N⁴-(4-(1,3-dimethyl-1H-pyrazol-4-yl)-5-methoxypyrimidin-2-yl)-N1-(2-(dimethylamino)ethyl)-N¹,N²-dimethylbenzene-1,2,4-triamine (290)

To the mixture of intermediate 20 (0.060 g, 0.25 mmol) and intermediate 85b (0.050 g, 0.25 mmol) in t-BuOH (20 mL) were added K₂CO₃ (0.100 g, 0.72 mmol), Pd₂(dba)₃ (10 mg, 0.01 mmol) and X-Phos (10 mg, 0.02 mmol). The mixture was stirred at 85° C. overnight. After cooling to room temperature and concentration, the residue was purified by flash column chromatography (dichloromethane/methanol=30/1-20/1) and preparative thin layer chromatography to give 290 (20 mg, 4.6% yield) as a yellow solid. LC-MS: m/z 425.3 [M+H]⁺.

### Procedure for the Syntheses of 291a-d

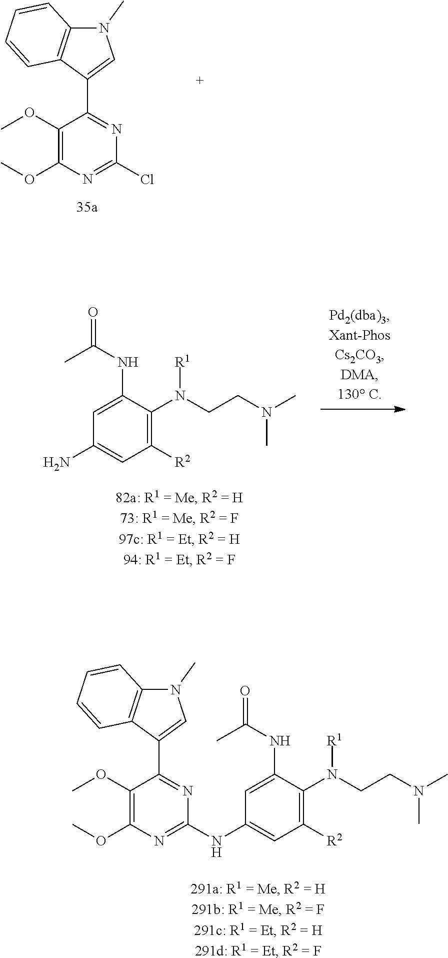

### N-(5-((4, 5-dimethoxy-6-(1-methyl-1H-indol-3-yl) pyrimidin-2-yl) amino)-2-((2-(dimethylamino) ethyl) (methyl) amino) phenyl)acetamide (291a)

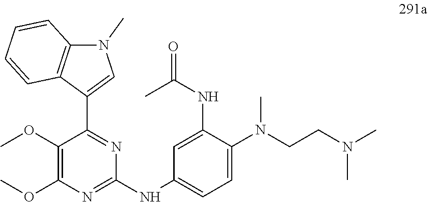

Synthesis of 291a was similar to that of 256a. The crude was purified by Pre-HPLC (MeCN/0.05% NH4OH in water from 0 to 100%) to afford 291a (53 mg, yield 34%) as a yellow solid. MS-ESI (M+H)⁺: 518.4. ¹H NMR (400 MHz, DMSO-d6) δ 10.07 (s, 1H), 9.04 (s, 1H), 8.54-8.51 (m, 2H), 8.44 (d, J=8.0 Hz, 1H), 7.46 (d, J=8.0 Hz, 1H), 7.33 (d, J=8.4 Hz, 1H), 7.24 (d, J=8.4 Hz, 1H), 7.21-7.17 (m, 1H), 7.13-7.10 (m, 1H), 4.07 (s, 3H), 3.87 (s, 3H), 3.75 (s, 3H), 2.81 (d, J=5.2 Hz, 2H), 2.66 (s, 3H), 2.26 (t, J=5.2 Hz, 2H), 2.22 (s, 6H), 2.12 (s, 3H).

### N-(5-((4, 5-dimethoxy-6-(1-methyl-1H-indol-3-yl)pyrimidin-2-yl)amino)-2-((2-(dimethylamino)ethyl)(methyl)amino)-3-fluorophenyl)acetamide (291b)

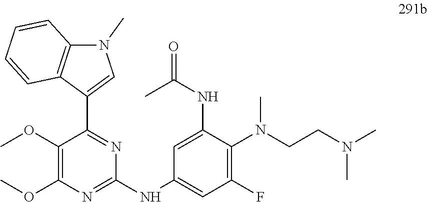

Synthesis of 291b was similar to that of 256a. The crude was purified by Pre-HPLC (MeCN/0.05% NH4OH in water from 0 to 100%) to afford 291b (53 mg, yield 30%) as a yellow solid. MS-ESI (M+H)⁺: 536.2. ¹H NMR (400 MHz, DMSO-d6) δ 10.49 (s, 1H), 8.97 (s, 1H), 8.77 (s, 1H), 8.55 (s, 1H), 8.47 (d, J=7.6 Hz, 1H), 7.49 (d, J=8.0 Hz, 1H), 7.32 (dd, J=14.4, 2.4 Hz, 1H), 7.22-7.20 (m, 1H), 7.16-7.13 (m, 1H), 4.10 (s, 3H), 3.90 (s, 3H), 3.76 (s, 3H), 3.01 (br, 2H), 2.77 (s, 3H), 2.26 (br, 8H), 2.14 (s, 3H).

### N-(5-((4, 5-dimethoxy-6-(1-methyl-1H-indol-3-yl) pyrimidin-2-yl) amino)-2-((2-(dimethylamino) ethyl) (ethyl) amino) phenyl) acetamide (291c)

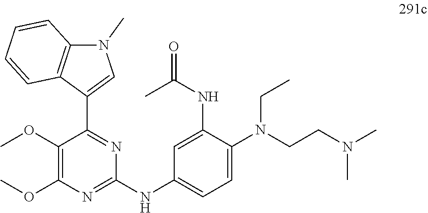

Synthesis of 291c was similar to that of 256a. The crude was purified by Pre-HPLC (MeCN/0.05% NH4OH in water from 0 to 100%) to afford 291c (59 mg, 0.11 mmol, yield 20%) as a yellow solid. MS-ESI (M+H)⁺: 532.3. ¹H NMR (400 MHz, DMSO-d6) δ 10.20 (s, 1H), 9.08 (s, 1H), 8.58-8.56 (m, 2H), 8.45 (d, J=8.0 Hz, 1H), 7.48 (d, J=8.0 Hz, 1H), 7.36 (d, J=8.0 Hz, 1H), 7.26 (d, J=8.4 Hz, 1H), 7.23-7.17 (m, 1H), 7.13-7.10 (m, 1H), 4.09 (s, 3H), 3.89 (s, 3H), 3.76 (s, 3H), 3.01-2.90 (m, 4H), 2.23-2.14 (m, 11H), 0.87 (t, J=7.2 Hz, 3H).

### N-(5-((4, 5-dimethoxy-6-(1-methyl-1H-indol-3-yl) pyrimidin-2-yl) amino)-2-((2-(dimethylamino) ethyl) (ethyl) amino)-3-fluorophenyl)acetamide (291d)

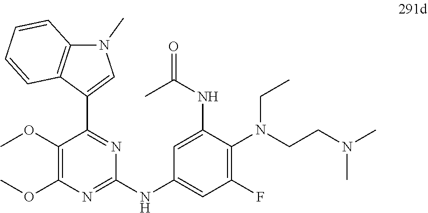

Synthesis of 291d was similar to that of 256a. The crude was purified by Pre-HPLC (MeCN/0.05% NH4OH in water from 0 to 100%) to afford 291d (55 mg, 0.1 mmol, yield 30.3%) as a yellow solid. MS-ESI (M+H)⁺: 550.7. ¹H NMR (400 MHz, DMSO-d6) δ 10.51 (s, 1H), 8.93 (s, 1H), 8.76 (s, 1H), 8.54 (s, 1H), 8.45 (d, J=7.6 Hz, 1H), 7.47 (d, J=8.0 Hz, 1H), 7.30 (dd, J=14.4, 2.4 Hz, 1H), 7.20 (t, J=7.2 Hz, 1H), 7.11 (t, J=7.4 Hz, 1H), 4.08 (s, 3H), 3.88 (s, 3H), 3.75 (s, 3H), 3.07-3.02 (m, 4H), 2.20 (br, 8H), 2.11 (s, 3H), 0.88 (t, J=7.2 Hz, 3H).

### Procedure for the Synthesis of 294 and 295

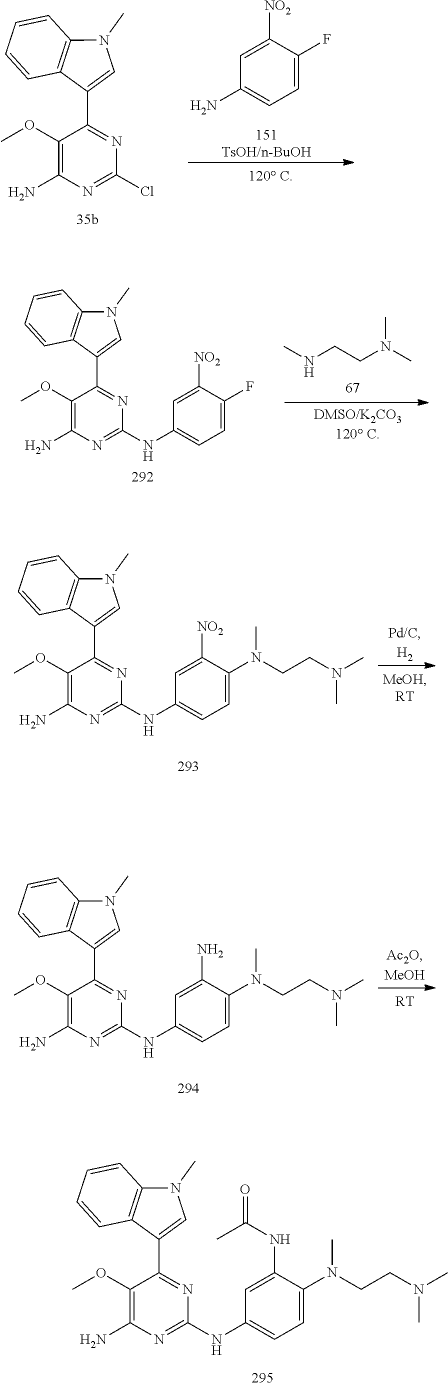

### N²-(4-fluoro-3-nitrophenyl)-5-methoxy-6-(1-methyl-1H-indol-3-yl) pyrimidine-2, 4-diamine (292)

To a solution of 35b (500 mg, 2.58 mmol) and 4-fluoro-3-nitroaniline 151 (512 mg, 3.28 mmol) in n-BuOH (15 mL) and was added TsOH (499 mg, 2.625 mmol). The mixture was stirred at 120° C. for 16 h under Ar. The mixture was concentrated to give a residue. The residue was purified by reverse phase column (0.1% FA/CAN=0-40%-60%) to afford 292 (700 mg) as a yellow solid. MS-ESI (M+H): 408.8.

### N²-(4-((2-(dimethylamino) ethyl) (methyl) amino)-3-nitrophenyl)-5-methoxy-6-(1-methyl-1H-indol-3-yl) pyrimidine-2, 4-diamine (293)

To a solution of 292 (450 mg, 1.1 mmol) in DMSO (10 mL) was added 67 (225 mg, 2.2 mmol) and K₂CO₃ (305 mg, 2.2 mmol). The mixture was stirred at 120° C. for 16 h under Ar. After cooling to rt, the mixture was purified by reverse phase column (0.1% FA/MeCN=0-30%-100%) to afford 293 (300 mg) as a red solid. MS-ESI (M+H)⁺: 491.3.

### N⁴-(4-amino-5-methoxy-6-(1-methyl-1H-indol-3-yl)pyrimidin-2-yl)-N¹-(2-(dimethylamino)ethyl)-N¹-methylbenzene-1,2,4-triamine (294)

To a solution of 293 (300 mg, 0.612 mmol) in methanol (20 mL) was added Pd/C (50 mg). The mixture was stirred at room temperature for 3 h under H₂. The mixture was filtered through Celite® pad and the filtrate was concentrated. The residue was purified by Prep-HPLC (0.1% FA/MeCN=0˜30%˜100%) to afford 294 (formic acid salt, 10 mg, yield 21.7%) as a green solid and 294 (100 mg, impure). MS-ESI (M+H)⁺: 461.3. ¹H NMR (400 MHz, DMSO-d6) δ 8.74 (d, J=8.0 Hz, 1H), 8.30 (s, 4H), 8.13 (s, 1H), 7.47 (d, J=8.2 Hz, 1H), 7.21 (t, J=7.5 Hz, 1H), 7.15-7.06 (m, 2H), 6.95 (dd, J=8.5, 2.1 Hz, 1H), 6.85 (d, J=8.5 Hz, 1H), 6.30 (s, 2H), 3.89 (s, 3H), 3.58 (s, 3H), 2.92 (t, J=6.3 Hz, 2H), 2.61-2.55 (m, 5H), 2.33 (s, 6H).

### N-(5-((4-amino-5-methoxy-6-(1-methyl-1H-indol-3-yl) pyrimidin-2-yl) amino)-2-((2-(dimethylamino) ethyl) (methyl) amino) phenyl) acetamide (295)

To a solution of 294 (100 mg, 0.217 mmol) in methanol (15 mL) was added Ac₂O (0.5 mL, 0.5M in methanol). The mixture was stirred at room temperature for 16 h. After concentration, the residue was purified by Prep-HPLC (0.05% NH₄OH/MeCN=0˜100%) to afford 295 (55 mg, yield 55%). MS-ESI (M+H)⁺: 503.3. ¹H NMR (400 MHz, DMSO-d6) δ 9.96 (s, 1H), 8.65 (d, J=8.1 Hz, 1H), 8.61 (s, 1H), 8.42 (s, 1H), 8.20 (s, 1H), 7.56 (dd, J=8.7, 2.5 Hz, 1H), 7.46 (d, J=8.2 Hz, 1H), 7.20 (t, J=7.1 Hz, 1H), 7.15 (d, J=8.7 Hz, 1H), 7.07 (t, J=7.2 Hz, 1H), 6.33 (s, 2H), 3.89 (s, 3H), 3.56 (s, 3H), 2.81 (s, 2H), 2.65 (s, 3H), 2.28 (s, 2H), 2.23 (s, 6H), 2.06 (s, 3H).

### Procedure for the Synthesis of 296

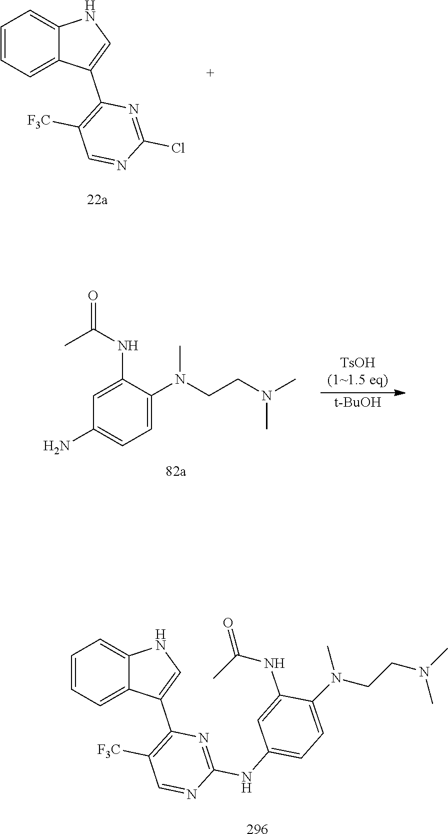

### N-(2-((2-(dimethylamino)ethyl)(methyl)amino)-5-((4-(1-methyl-1H-indol-3-yl)-5-(trifluoromethyl)pyrimidin-2-yl)amino)phenyl)acetamide (296)

A mixture of 22a (0.18 g, 0.60 mmol), 82a (0.10 g, 0.40 mmol) and TsOH (0.09 g, 0.47 mmol) in t-BuOH (15 mL) was stirred at 70° C. overnight. After cooling to room temperature, the mixture was neutralized with saturated NaHCO₃ and concentrated under reduced pressure. The residue was dissolved in ethyl acetate (40 mL), and washed with H₂O (10 mL*3), dried with Na₂SO₄. After concentration, the residue was purified by preparative thin layer chromatography (dichloromethane/methanol=12/1) to give the title product 296 (31 mg, yield 14.8%) as a gray solid. MS-ESI (M+H)⁺: 512.6. ¹H NMR (500 MHz, DMSO-d₆) δ 11.97 (s, 2H), 10.01 (s, 2H), 9.68 (s, 2H), 8.71 (s, 2H), 8.32 (s, 4H), 7.88 (d, J=2.9 Hz, 2H), 7.56 (dd, J=8.6, 2.6 Hz, 2H), 7.50 (d, J=8.1 Hz, 2H), 7.21 (t, J=7.5 Hz, 2H), 7.17 (d, J=8.7 Hz, 2H), 7.13 (t, J=7.7 Hz, 2H), 3.16 (s, 8H), 2.66 (s, 12H), 2.57 (s, 6H), 2.23 (s, 6H).

### Procedure for the Synthesis of 297

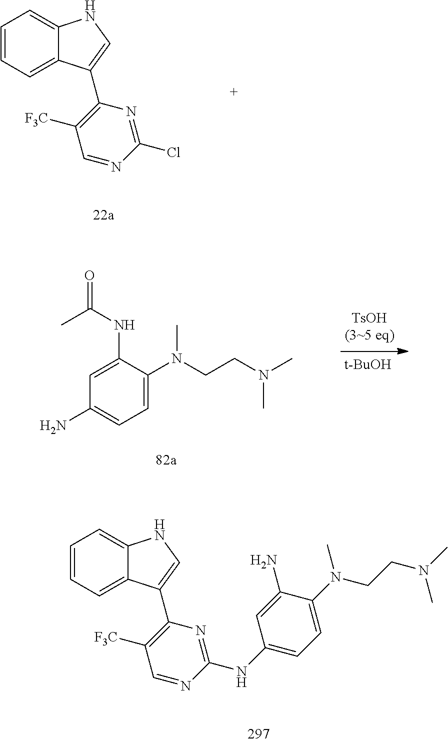

### N⁴-(4-(1H-indol-3-yl)-5-(trifluoromethyl)pyrimidin-2-yl)-N¹-(2-(dimethylamino)ethyl)-N¹-methylbenzene-1,2,4-triamine (297)

A mixture of 22a (0.20 g, 0.67 mmol), 82a (0.17 g, 0.68 mmol) and TsOH (0.36 g, 2.09 mmol) in t-BuOH (15 mL) was stirred at 100° C. overnight. After cooling to room temperature, the mixture was neutralized with saturated NaHCO₃ and concentrated under reduced pressure. The residue was dissolved in dichloromethane (40 mL), and washed with H₂O (10 mL*2), dried with Na₂SO₄. After concentration, the residue was purified by flash column chromatography (dichloromethane/methanol=12/1) to give the desired product 297 (38 mg, yield 12.0%) as a gray solid. MS-ESI (M+H)⁺: 470.5. ¹H NMR (500 MHz, DMSO-d₆) δ 11.93 (s, 1H), 9.75 (s, 1H), 8.67 (s, 1H), 8.34 (s, 1H), 7.87 (s, 1H), 7.50 (d, J=8.0 Hz, 1H), 7.21 (t, J=7.5 Hz, 1H), 7.15-7.09 (m, 1H), 7.02 (s, 1H), 6.98-6.93 (m, 1H), 6.90 (d, J=8.4 Hz, 1H), 3.00 (d, J=5.7 Hz, 2H), 2.86 (s, 2H), 2.54 (s, 3H), 2.50 (s, 6H).

### Procedure for the Synthesis of 298

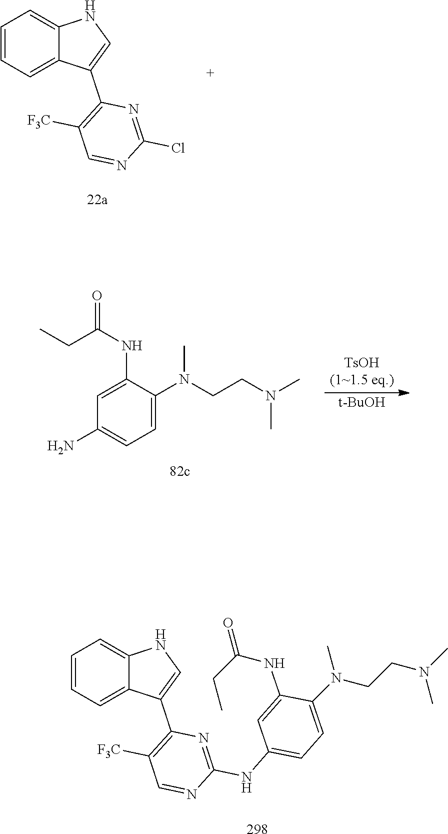

### N-(5-((4-(1H-indol-3-yl)-5-(trifluoromethyl)pyrimidin-2-yl)amino)-2-((2-(dimethylamino)ethyl)(methyl)amino)phenyl)propionamide (298)

Synthesis of 298 was similar to that of 296. The residue was purified by preparative thin layer chromatography (dichloromethane/methanol=12/1) to give the title product 298 (14 mg, yield 4.0%) as a gray solid. MS-ESI (M+H)⁺: 526.6. ¹H NMR (500 MHz, DMSO-d₆) δ 12.02 (s, 1H), 10.76 (s, 1H), 9.99 (s, 1H), 9.64 (s, 1H), 8.70 (s, 1H), 8.33 (s, 2H), 7.88 (s, 1H), 7.56 (d, J=7.6 Hz, 1H), 7.50 (d, J=7.8 Hz, 1H), 7.21 (s, 1H), 7.17 (d, J=8.2 Hz, 1H), 7.12 (s, 1H), 3.14 (s, 4H), 2.63 (s, 4H), 2.57 (s, 6H), 1.08 (t, J=6.8 Hz, 3H).

### Procedure for the Synthesis of 299a and 299b

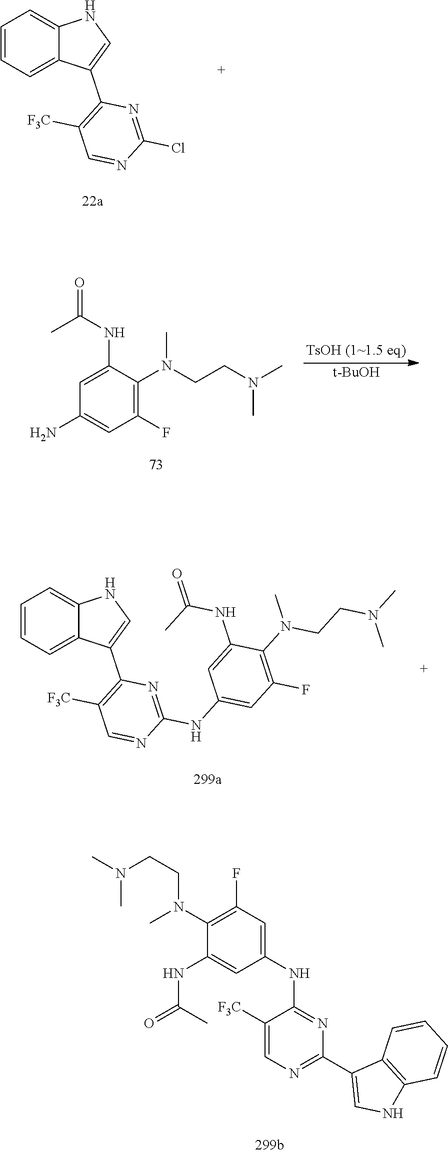

### N-(5-((4-(1H-indol-3-yl)-5-(trifluoromethyl)pyrimidin-2-yl)amino)-2-((2-(dimethylamino)ethyl)(methyl)amino)-3-fluorophenyl)acetamide (299a) & N-(5-((2-(1H-indol-3-yl)-5-(trifluoromethyl)pyrimidin-4-yl)amino)-2-((2-(dimethylamino)ethyl)(methyl)amino)-3-fluorophenyl)acetamide (299b)

Synthesis of 299a and 299b were similar to that of 296. The residue was purified by preparative thin layer chromatography (dichloromethane/methanol=12/1) to give the title product 299a (11 mg, yield 5.5%) as a gray solid and products 299b (9 mg, yield 4.5%) as a gray solid.

For 299a: MS-ESI (M+H)⁺: 530.5. ¹H NMR (500 MHz, DMSO-d₆) δ 11.94 (s, 1H), 10.27 (s, 1H), 9.88 (s, 1H), 8.78 (s, 1H), 8.38 (s, 1H), 8.29 (s, 1H), 7.90 (s, 1H), 7.74 (dd, J=14.9, 2.2 Hz, 1H), 7.52 (d, J=8.1 Hz, 1H), 7.23 (t, J=7.9 Hz, 1H), 7.14 (t, J=7.4 Hz, 1H), 3.12 (s, 2H), 2.70 (s, 3H), 2.55 (s, 2H), 2.51 (d, J=3.4 Hz, 6H), 2.18 (s, 3H);

For 299b: MS-ESI (M+H)⁺: 530.3. ¹H NMR (500 MHz, DMSO-d₆) δ 11.85 (d, J=3.0 Hz, 1H), 9.96 (s, 1H), 8.97 (s, 1H), 8.64 (s, 1H), 8.39 (s, 1H), 8.25 (d, J=2.9 Hz, 1H), 8.13 (d, J=8.0 Hz, 1H), 7.44 (d, J=8.1 Hz, 1H), 7.28-7.19 (m, 2H), 7.15 (t, J=7.6 Hz, 1H), 6.97 (t, J=7.5 Hz, 1H), 3.22 (s, 2H), 2.79 (s, 3H), 2.60 (s, 2H), 2.54 (s, 6H), 2.18 (s, 3H).

### Procedure for the Syntheses of 300a and 300b

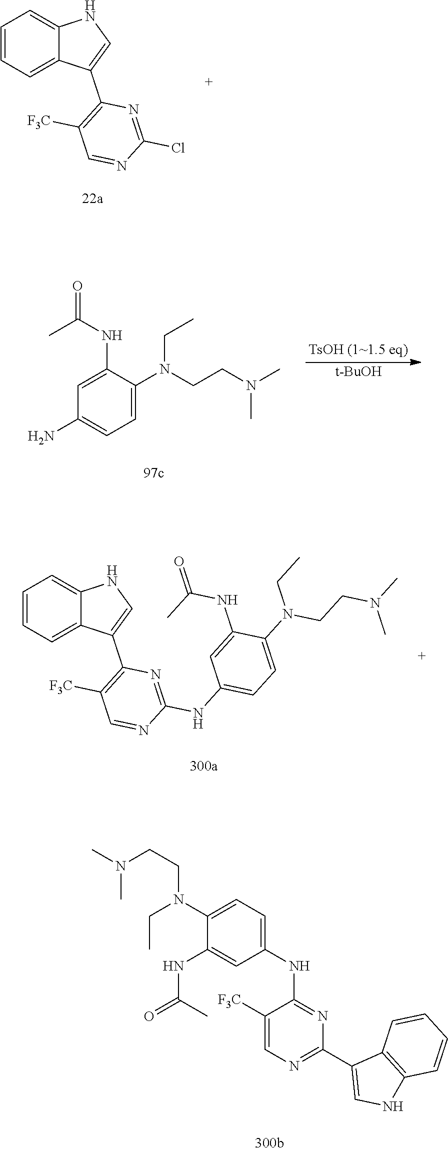

### N-(5-((4-(1H-indol-3-yl)-5-(trifluoromethyl)pyrimidin-2-yl)amino)-2-((2-(dimethylamino)ethyl)(ethyl)amino)phenyl)acetamide (300a) & N-(5-((2-(1H-indol-3-yl)-5-(trifluoromethyl)pyrimidin-4-yl)amino)-2-((2-(dimethylamino)ethyl)(ethyl)amino)phenyl)acetamide (300b)

Synthesis of 300a and 300b were similar to that of 296. The residue was purified by preparative thin layer chromatography (dichloromethane/methanol=12/1) to give the title product 300a (33 mg, yield 8.9%) as a gray solid and products 300b (18 mg, yield 5.2%) as a gray solid.

For 300a: MS-ESI (M+H)⁺: 526.6. ¹H NMR (500 MHz, DMSO-d₆) δ 11.89 (d, J=3.1 Hz, 1H), 10.02 (s, 1H), 9.99 (s, 1H), 8.71 (s, 1H), 8.40 (s, 1H), 8.37-8.22 (m, 1H), 7.89 (d, J=2.8 Hz, 1H), 7.57 (dd, J=8.6, 2.6 Hz, 1H), 7.50 (d, J=8.1 Hz, 1H), 7.22 (d, J=2.3 Hz, 1H), 7.20 (d, J=8.2 Hz, 1H), 7.09 (t, J=7.5 Hz, 1H), 3.05-2.97 (m, 2H), 2.95 (t, J=7.1 Hz, 2H), 2.47-2.37 (m, 2H), 2.33 (s, 6H), 2.09 (s, 3H), 0.86 (t, J=7.1 Hz, 3H);

For 300b: MS-ESI (M+H)⁺: 526.5. ¹H NMR (500 MHz, DMSO-d₆) δ 11.81 (d, J=3.0 Hz, 1H), 9.95 (s, 1H), 8.85 (s, 1H), 8.59 (s, 1H), 8.44-8.33 (m, 1H), 8.23 (d, J=2.9 Hz, 1H), 8.07 (d, J=8.0 Hz, 1H), 7.41 (d, J=8.1 Hz, 1H), 7.36 (d, J=8.5 Hz, 1H), 7.25 (dd, J=8.6, 2.5 Hz, 1H), 7.11 (t, J=7.6 Hz, 1H), 6.93 (t, J=7.6 Hz, 1H), 3.15 (s, 2H), 3.05 (q, J=7.1 Hz, 2H), 2.46 (s, 4H), 2.15 (s, 3H), 0.95 (t, J=7.0 Hz, 3H).

### Procedure for the Synthesis of 301

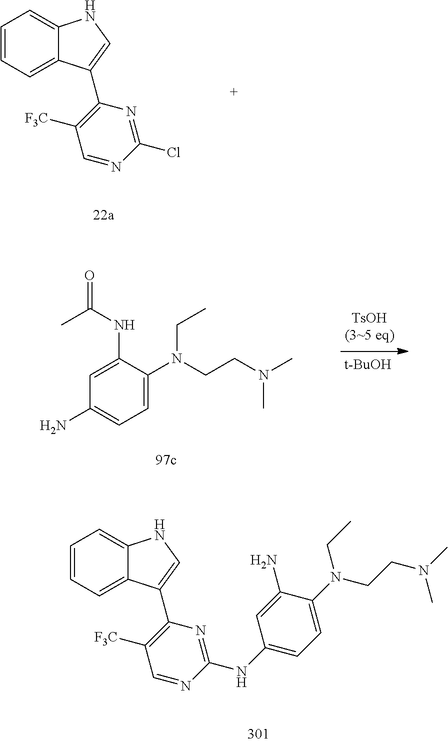

### N⁴-(4-(1H-indol-3-yl)-5-(trifluoromethyl)pyrimidin-2-yl)-N¹-(2-(dimethylamino)ethyl)-N-ethylbenzene-1,2,4-triamine (301)

Synthesis of 301 was similar to that of 297. The residue was purified by preparative thin layer chromatography (dichloromethane/methanol=12/1) to give the title product 301 (85 mg, yield 26.2%) as a gray solid. MS-ESI (M+H)⁺: 484.3. ¹H NMR (500 MHz, DMSO-d₆) δ 11.93 (s, 1H), 9.76 (s, 1H), 8.68 (s, 1H), 8.35 (s, 1H), 7.88 (s, 1H), 7.50 (d, J=8.0 Hz, 1H), 7.21 (t, J=7.4 Hz, 1H), 7.15-7.08 (m, 1H), 7.04 (s, 1H), 6.98 (d, J=8.3 Hz, 1H), 6.93 (d, J=8.4 Hz, 1H), 3.17 (s, 1H), 3.13 (s, 2H), 2.99-2.86 (m, 4H), 2.62 (s, 6H), 0.91 (t, J=6.8 Hz, 3H).

### Procedure for the Synthesis of 302

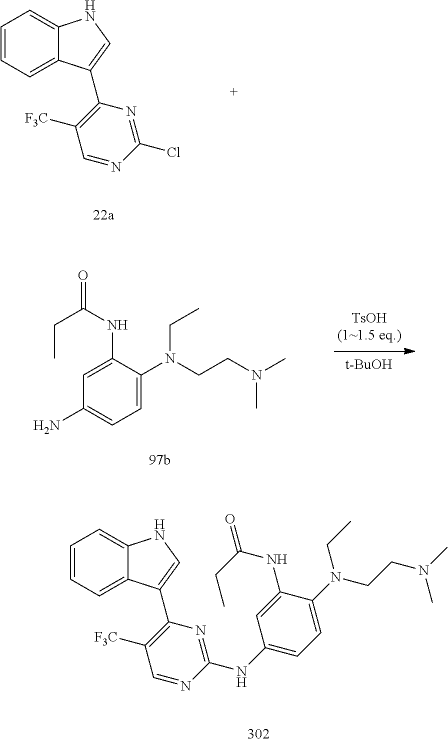

### N-(5-((4-(1H-indol-3-yl)-5-(trifluoromethyl)pyrimidin-2-yl)amino)-2-((2-(dimethylamino)ethyl)(ethyl)amino)phenyl)propionamide (302)

Synthesis of 302 was similar to that of 296. The residue was purified by preparative thin layer chromatography (dichloromethane/methanol=12/1) to give the title product 296 (27 mg, yield 7.1%) as a gray solid. MS-ESI (M+H)⁺: 540.6. ¹H NMR (500 MHz, DMSO-d₆) δ 11.90 (s, 1H), 10.02 (s, 1H), 9.82 (s, 1H), 8.72 (s, 1H), 8.36 (d, J=20.9 Hz, 2H), 7.89 (s, 1H), 7.58 (d, J=10.8 Hz, 1H), 7.50 (d, J=8.1 Hz, 1H), 7.23 (s, 1H), 7.19 (s, 1H), 7.09 (s, 1H), 3.06 (s, 2H), 2.95 (q, J=7.0 Hz, 2H), 2.58 (s, 2H), 2.41 (s, 6H), 1.11 (t, J=7.5 Hz, 3H), 0.87 (t, J=7.0 Hz, 3H).

### Procedure for the Syntheses of 303a and 303b

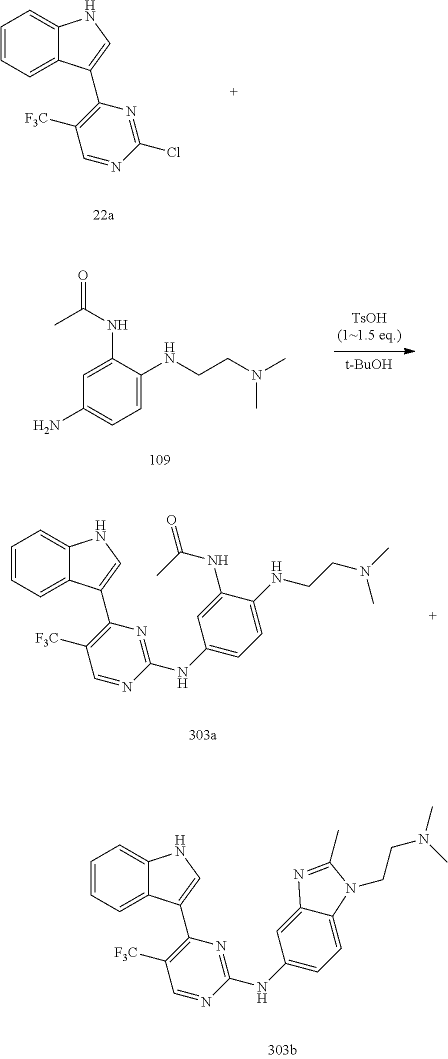

### N-(5-((4-(1H-indol-3-yl)-5-(trifluoromethyl)pyrimidin-2-yl)amino)-2-((2-(dimethylamino)ethyl)amino)phenyl)acetamide (303a) & N¹—N-(4-(1H-indol-3-yl)-5-(trifluoromethyl)pyrimidin-2-yl)-1-(2-(dimethylamino)ethyl)-2-methyl-1H-benzo[d]imidazol-5-amine (303b)

Synthesis of 303a and 303b was similar to that of 296. The residue was purified by preparative thin layer chromatography (dichloromethane/methanol=12/1) to give the title product 303a (5 mg, yield 1.5%) as a gray solid and 303b (7 mg, yield 2.2%) as a gray solid.

For 303a: MS-ESI (M+H)⁺: 498.4.

For 303b: MS-ESI (M+H)⁺: 480.4. ¹H NMR (500 MHz, DMSO-d₆) δ 11.86 (s, 1H), 9.96 (s, 1H), 8.71 (s, 1H), 7.94 (s, 1H), 7.88 (s, 1H), 7.53 (dd, J=8.7, 1.8 Hz, 1H), 7.49 (d, J=8.2 Hz, 1H), 7.43 (d, J=8.6 Hz, 1H), 7.23-7.17 (m, 1H), 7.05 (s, 1H), 4.27 (t, J=6.5 Hz, 2H), 2.64 (s, 2H), 2.54 (s, 3H), 2.26 (s, 6H).

### Procedure for the Synthesis of 304

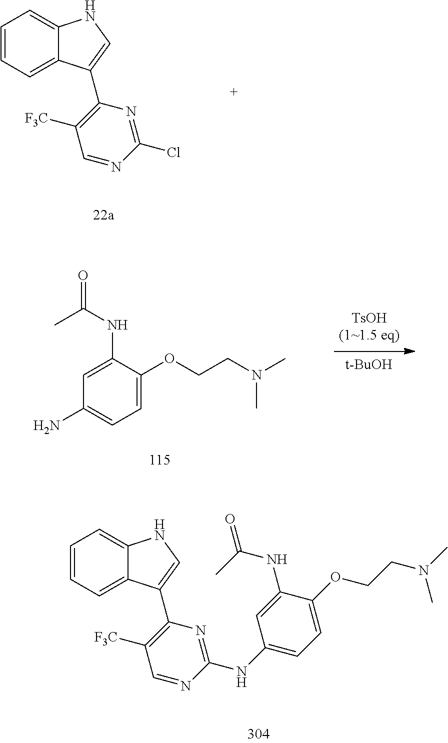

### N-(5-((4-(1H-indol-3-yl)-5-(trifluoromethyl)pyrimidin-2-yl) amino)-2-(2-(dimethylamino)ethoxy)phenyl)acetamide (304)

A mixture of 22a (0.20 g, 0.67 mmol), 115 (0.17 g, 0.72 mmol), and TsOH (0.14 g, 0.81 mmol) in t-BuOH (15 mL) was stirred at 100° C. for 7 h. After cooling to room temperature, the mixture was neutralized with saturated NaHCO₃ and concentrated under reduced pressure. The residue was dissolved in ethyl acetate (40 mL), and washed with H₂O (15 mL*2), dried with Na₂SO₄. After concentration, the residue was purified by preparative thin layer chromatography (dichloromethane/methanol=12/1) to give the desired product 304 (5 mg, yield 1.5%) as a gray solid. MS-ESI (M+H)⁺: 499.6.

### Procedure for the Synthesis of 305

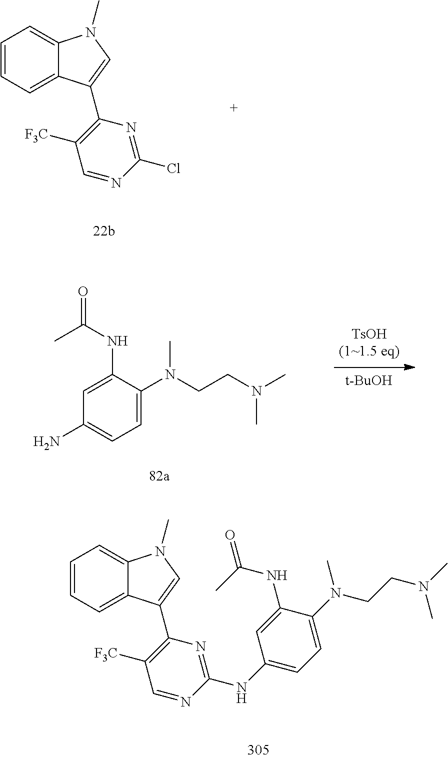

### N-(2-((2-(dimethylamino)ethyl)(methyl)amino)-5-((4-(1-methyl-1H-indol-3-yl)-5-(trifluoromethyl)pyrimidin-2-yl)amino)phenyl)acetamide (305)

A mixture of intermediate 22b (0.21 g, 0.67 mmol), intermediate 82a (0.16 g, 0.64 mmol) and TsOH (0.16 g, 0.93 mmol) in t-BuOH (15 mL) was stirred at 110° C. for 7 h. After cooling to room temperature, the mixture was neutralized with saturated NaHCO₃ and concentrated under reduced pressure. The residue was dissolved in ethyl acetate (40 mL), and washed with H₂O (15 mL*3), dried with Na₂SO₄. After concentration, the residue was purified by flash column chromatography (dichloromethane/methanol=15/1) to give the desired product 305 (143 mg, yield 42.4%) as a gray solid. LC-MS: m/z 526.5 [M+H]⁺. ¹H NMR (500 MHz, DMSO-d₆) δ 10.57 (s, 1H), 10.03 (s, 1H), 9.60 (s, 1H), 8.71 (s, 1H), 8.32 (s, 1H), 7.91 (s, 1H), 7.57 (dd, J=8.6, 2.5 Hz, 1H), 7.54 (d, J=8.3 Hz, 1H), 7.29 (ddd, J=8.2, 6.9, 1.2 Hz, 1H), 7.18 (dd, J=12.4, 8.0 Hz, 2H), 3.92 (s, 3H), 3.29-3.15 (m, 4H), 2.73 (s, 6H), 2.56 (s, 3H), 2.24 (s, 3H).

### Procedure for the Synthesis of 306

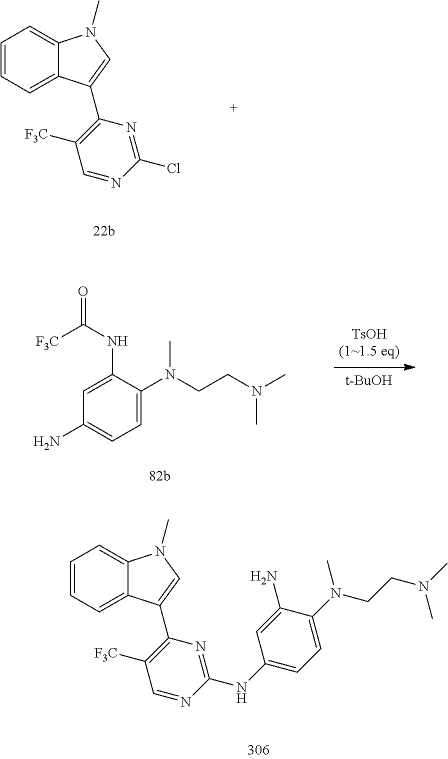

### N¹-(2-(dimethylamino)ethyl)-N¹-methyl-N⁴-(4-(1-methyl-1H-indol-3-yl)-5-(trifluoromethyl)pyrimidin-2-yl)benzene-1,2,4-triamine (306)

Synthesis of 306 was similar to that of 305. The residue was purified by preparative thin layer chromatography (dichloromethane/methanol=12/1) to give the title product 306 (103 mg, yield 41.5%) as a gray solid. MS-ESI (M+H)⁺: 483.9. ¹H NMR (500 MHz, DMSO-d₆) δ 9.75 (s, 1H), 8.67 (s, 1H), 8.38 (s, 1H), 7.88 (s, 1H), 7.53 (d, J=8.2 Hz, 1H), 7.28 (t, J=7.6 Hz, 1H), 7.17 (t, J=7.6 Hz, 1H), 7.02 (s, 1H), 6.95 (dd, J=8.4, 2.4 Hz, 1H), 6.90 (d, J=8.5 Hz, 1H), 3.91 (s, 3H), 3.06 (d, J=5.6 Hz, 2H), 3.02 (d, J=6.0 Hz, 2H), 2.52 (s, 3H).

### Procedure for the Synthesis of 307

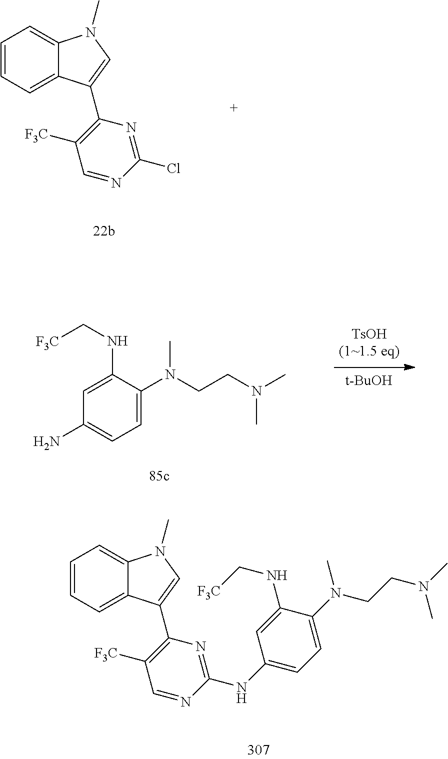

### N-(2-((2-(dimethylamino)ethyl)(methyl)amino)-5-((4-(1-methyl-1H-indol-3-yl)-5-(trifluoromethyl)pyrimidin-2-yl)amino)phenyl)acetamide (307)

Synthesis of 307 was similar to that of 305 by using intermediate 22b (150 mg, 0.48 mmol) and intermediate 85c (144 mg, 0.50 mmol). The residue was purified by flash column chromatography (dichloromethane/methanol=20/1) to give the title product 307 (98 mg, yield 36.0%) as a gray solid.

### Procedure for the Synthesis of 308

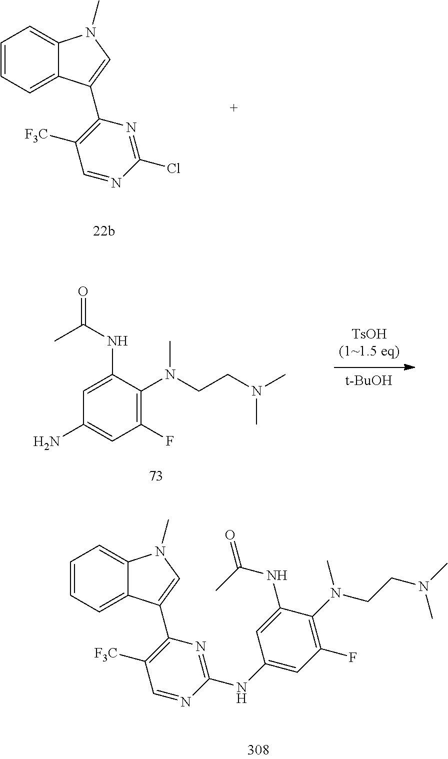

### N-(2-((2-(dimethylamino)ethyl)(methyl)amino)-3-fluoro-5-((4-(1-methyl-1H-indol-3-yl)-5-(trifluoromethyl)pyrimidin-2-yl)amino)phenyl)acetamide (308)

Synthesis of 308 was similar to that of 305. The residue was purified by preparative thin layer chromatography (dichloromethane/methanol=12/1) to give the title product 308 (38 mg, yield 18.2%) as a gray solid. MS-ESI (M+H)⁺: 544.5. ¹H NMR (500 MHz, DMSO-d₆) δ 10.25 (s, 2H), 8.77 (s, 1H), 8.41 (s, 1H), 8.29 (s, 1H), 7.92 (s, 1H), 7.71 (dd, J=14.8, 2.4 Hz, 1H), 7.55 (d, J=8.1 Hz, 1H), 7.30 (ddd, J=8.2, 7.0, 1.2 Hz, 1H), 7.19 (t, J=7.6 Hz, 1H), 3.93 (s, 3H), 3.02 (t, J=5.6 Hz, 2H), 2.72 (d, J=1.4 Hz, 3H), 2.40 (s, 2H), 2.31 (s, 6H), 2.10 (s, 3H).

### Procedure for the Synthesis of 309

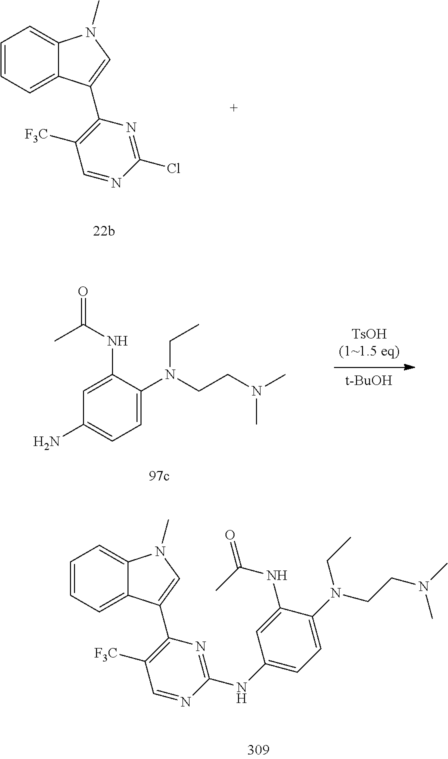

### N-(2-((2-(dimethylamino)ethyl)(ethyl)amino)-5-((4-(1-methyl-1H-indol-3-yl)-5-(trifluoromethyl)pyrimidin-2-yl)amino)phenyl)acetamide (309)

Synthesis of 309 was similar to that of 305. The residue was purified by preparative thin layer chromatography (dichloromethane/methanol=12/1) to give the title product 309 (60 mg, yield 23.1%) as a gray solid. MS-ESI (M+H)⁺: 540.6. ¹H NMR (500 MHz, DMSO-d₆) δ 10.03 (s, 1H), 9.93 (s, 1H), 8.71 (s, 1H), 7.91 (s, 1H), 7.57 (dd, J=8.7, 2.6 Hz, 1H), 7.53 (d, J=8.2 Hz, 1H), 7.28 (t, J=7.6 Hz, 1H), 7.21 (d, J=8.7 Hz, 1H), 7.14 (s, 1H), 3.92 (s, 3H), 3.02 (s, 2H), 2.95 (q, J=7.0 Hz, 2H), 2.38 (s, 6H), 2.11 (s, 3H), 0.87 (t, J=7.0 Hz, 3H).

### Procedure for the Synthesis of 310

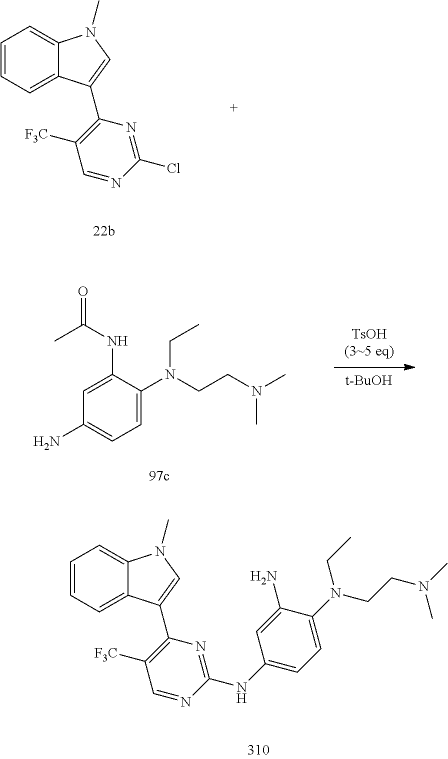

### N¹-(2-(dimethylamino)ethyl)-N¹-ethyl-N⁴-(4-(1-methyl-1H-indol-3-yl)-5-(trifluoromethyl)pyrimidin-2-yl)benzene-1,2,4-triamine (310)

Synthesis of 310 was similar to that of 297. The residue was purified by preparative thin layer chromatography (dichloromethane/methanol=12/1) to give the title product 310 (50 mg, yield 20.6%) as a gray solid. MS-ESI (M+H)⁺: 498.4. ¹H NMR (500 MHz, DMSO-d₆) δ 9.77 (s, 1H), 8.68 (s, 1H), 8.37 (s, 1H), 7.89 (s, 1H), 7.54 (d, J=8.1 Hz, 1H), 7.28 (t, J=7.4 Hz, 1H), 7.16 (s, 1H), 7.04 (s, 1H), 6.97 (d, J=8.3 Hz, 1H), 6.93 (d, J=8.2 Hz, 1H), 3.92 (s, 3H), 3.16 (s, 2H), 3.03 (s, 2H), 2.91 (d, J=6.8 Hz, 2H), 2.69 (s, 6H), 0.91 (t, J=6.6 Hz, 3H).

### Procedure for the Synthesis of 313

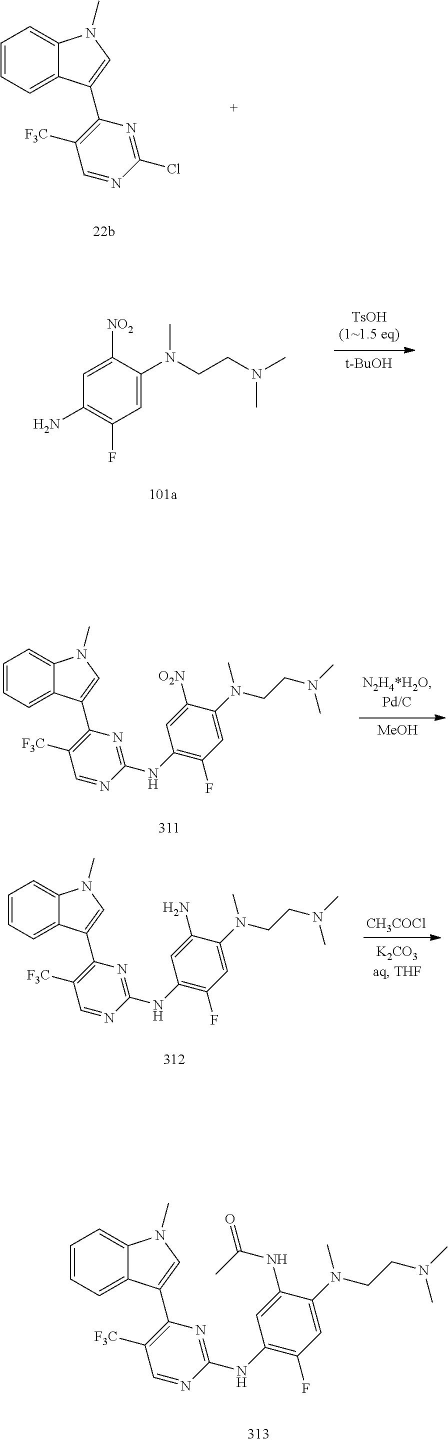

### N¹-(2-(dimethylamino)ethyl)-5-fluoro-N¹-methyl-N⁴-(4-(1-methyl-1H-indol-3-yl)-5-(trifluoromethyl)pyrimidin-2-yl)-2-nitrobenzene-1,4-diamine (311)

Synthesis of 311 was similar to that of 305 using 22b (112.4 mg, 0.36 mmol) and 101a (87 mg, 0.34 mmol). The reaction mixture was concentrated and the residue was directly used in the next step without further purification.

### N¹-(2-(dimethylamino)ethyl)-5-fluoro-N¹-methyl-N⁴-(4-(1-methyl-1H-indol-3-yl)-5-(trifluoromethyl)pyrimidin-2-yl)benzene-1,2,4-triamine (312)

A mixture of above crude 311, Pd/C (5% on activated carbon, 92.1 mg, 0.04 mmol) and hydrazine hydrate (80%, 5 ml, 80 mmol) in methanol (20 ml) was heated at refluxing temperature for 3 hrs. After cooling to room temperature, the reaction mixtures was filtered through Celite® and the filtrates were concentrated to remove methanol. The residual was extracted with ethyl acetate. The organic layer was separated, washed with water, dried over Na₂SO₄, filtered and concentrated to dryness. The crude was purified by flash column chromatography (eluting with methanol/dichloromethane, from 1/50 to 1/4) to give the title compound 312 (40 mg, yield 23.5% over two steps) as an oil. LC-MS: m/z 502.5 [M+H]⁺.

### N-(2-((2-(dimethylamino)ethyl)(methyl)amino)-4-fluoro-5-((4-(1-methyl-1H-indol-3-yl)-5-(trifluoromethyl)pyrimidin-2-yl)amino)phenyl)acetamide (313)

Acetyl chloride (20 mg, 0.25 mmol) was dropped to the mixture of 312 (30 mg, 0.06 mmol) and saturated K₂CO₃ aqueous (0.5 ml) in tetrahydrofuran (1 mL) at 0° C. The mixture was stirred for 1 hr. Then the mixture was concentrated and diluted with ethyl acetate (50 ml). The organic layers were combined, washed with brine (50 ml*3), and then concentrated under reduced pressure. The residue was purified by preparative thin layer chromatography (dichloromethane/methanol=20/1) to give the title compound 313 (20 mg, yield 61.5%) as a gray solid. LC-MS: m/z 544.5 [M+H]⁺.

### Procedure for the Syntheses of 314a and 314b

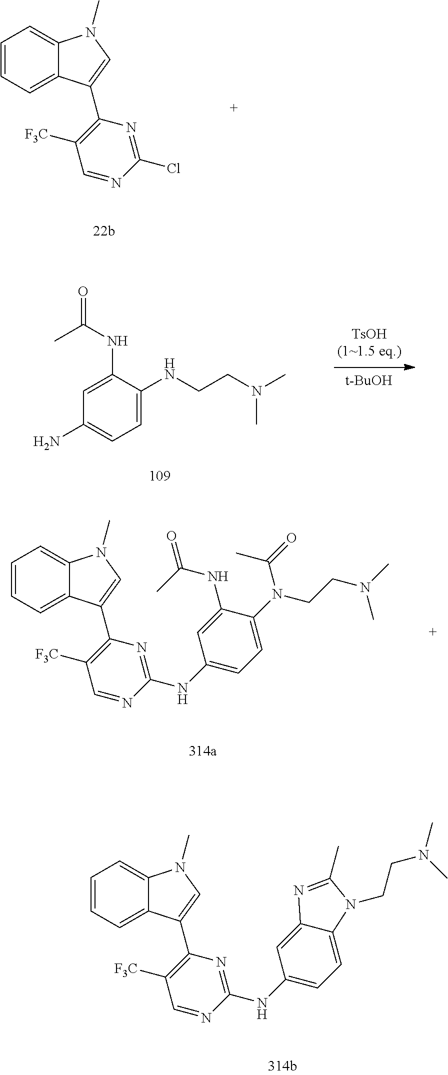

### N-(2-acetamido-4-((4-(1-methyl-1H-indol-3-yl)-5-(trifluoromethyl)pyrimidin-2-yl)amino)phenyl)-N-(2-(dimethylamino)ethyl)acetamide (314a) & 1-(2-(dimethylamino)ethyl)-2-methyl-N-(4-(1-methyl-1H-indol-3-yl)-5-(trifluoromethyl)pyrimidin-2-yl)-1H-benzo[d]imidazol-5-amine (314b)

Synthesis of 314a and 314b were similar to that of 305. The residue was purified by flash column chromatography (dichloromethane/methanol=15/1) to give the title product 314a (29 mg, yield 8.2%) as a gray solid and products 314b (47 mg, yield 14.8%) as a gray solid.

For 314a: MS-ESI (M+H)⁺: 554.6. ¹H NMR (500 MHz, DMSO-d₆) δ 10.84 (s, 1H), 10.25 (s, 1H), 8.77 (s, 1H), 8.38 (s, 1H), 8.32 (s, 1H), 7.92 (s, 1H), 7.75 (d, J=6.9 Hz, 1H), 7.55 (d, J=8.2 Hz, 1H), 7.29 (t, J=7.4 Hz, 1H), 7.23 (d, J=8.6 Hz, 1H), 7.17 (t, J=7.2 Hz, 1H), 4.52 (t, J=11.8 Hz, 1H), 3.92 (s, 3H), 3.09 (d, J=14.5 Hz, 1H), 2.41-2.27 (m, 2H), 2.24 (s, 6H), 2.00 (s, 3H), 1.60 (s, 3H);

For 314b: MS-ESI (M+H)⁺: 494.4. ¹H NMR (500 MHz, DMSO-d₆) δ 9.96 (s, 1H), 8.71 (s, 1H), 7.93 (s, 1H), 7.89 (s, 1H), 7.56-7.48 (m, 2H), 7.41 (d, J=8.6 Hz, 1H), 7.31-7.21 (m, 1H), 7.09 (s, 1H), 4.25 (t, J=6.5 Hz, 2H), 3.92 (s, 3H), 2.59 (t, J=6.4 Hz, 2H), 2.53 (s, 3H), 2.23 (s, 6H).

### Procedure for the Synthesis of 315

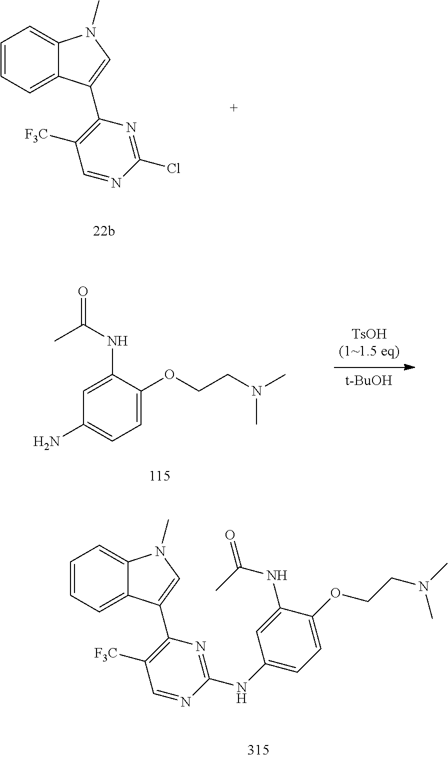

### N-(2-(2-(dimethylamino)ethoxy)-5-((4-(1-methyl-1H-indol-3-yl)-5-(trifluoromethyl)pyrimidin-2-yl)amino)phenyl)acetamide (315)

Synthesis of 315 was similar to that of 305. The residue was purified by preparative thin layer chromatography (dichloromethane/methanol=12/1) to give the title product 315 (56 mg, yield 17.0%) as a gray solid. MS-ESI (M+H)⁺: 513.6. ¹H NMR (500 MHz, DMSO-d₆) δ 9.95 (s, 1H), 9.66 (s, 1H), 8.69 (s, 1H), 8.31 (s, 2H), 7.90 (s, 1H), 7.52 (dd, J=15.5, 8.1 Hz, 2H), 7.28 (t, J=7.3 Hz, 1H), 7.16 (s, 1H), 7.03 (d, J=8.5 Hz, 1H), 4.16 (s, 2H), 3.91 (s, 3H), 2.87 (s, 2H), 2.44 (s, 6H), 2.11 (s, 3H).

### Procedure for the Synthesis of 316

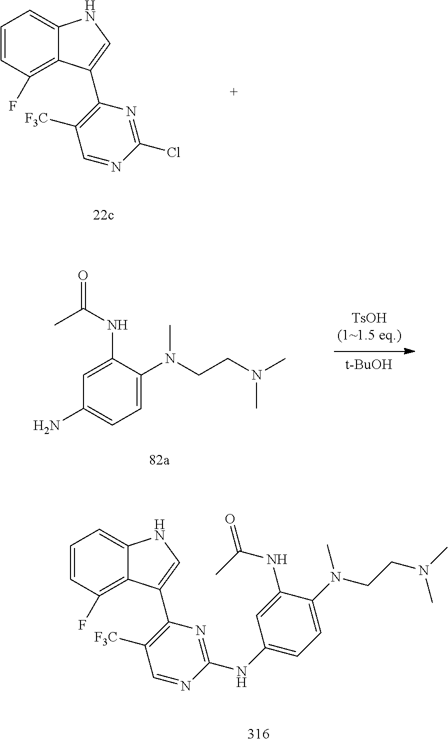

### N-(2-((2-(dimethylamino)ethyl)(methyl)amino)-5-((4-(4-fluoro-1H-indol-3-yl)-5-(trifluoromethyl)pyrimidin-2-yl)amino)phenyl)acetamide (316)

Synthesis of 316 was similar to that of 296. The residue was purified by preparative thin layer chromatography (dichloromethane/methanol=12/1) to give the title product 316 (50 mg, yield 19.9%) as a gray solid. ¹H NMR (500 MHz, DMSO-d₆) δ 11.96 (s, 1H), 10.13 (s, 1H), 9.75 (s, 1H), 8.78 (s, 1H), 8.32 (s, 1H), 7.64 (s, 1H), 7.57 (s, 1H), 7.34 (d, J=8.1 Hz, 1H), 7.16 (dt, J=17.8, 8.7 Hz, 2H), 6.90-6.77 (m, 1H), 3.00 (s, 2H), 2.81 (s, 2H), 2.56 (s, 3H), 2.49 (s, 6H), 2.16 (s, 3H).

### Procedure for the Synthesis of 317

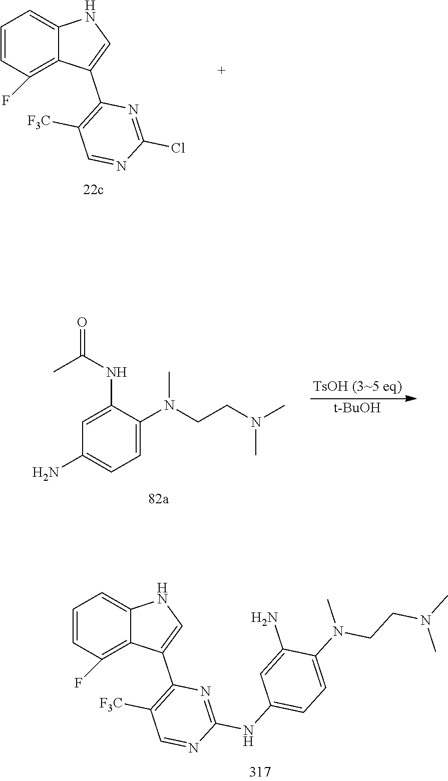

### N¹-(2-(dimethylamino)ethyl)-N⁴-(4-(4-fluoro-1H-indol-3-yl)-5-(trifluoromethyl)pyrimidin-2-yl)-N¹-methylbenzene-1,2,4-triamine (317)

Synthesis of 317 was similar to that of 297. The residue was purified by preparative thin layer chromatography (dichloromethane/methanol=12/1) to give the title product 317 (62 mg, yield 20.1%) as a gray solid. ¹H NMR (500 MHz, DMSO-d₆) δ 12.00 (s, 1H), 9.90 (s, 1H), 8.74 (s, 1H), 7.62 (s, 1H), 7.34 (d, J=8.2 Hz, 1H), 7.16 (td, J=7.9, 5.3 Hz, 1H), 6.98 (s, 2H), 6.92-6.79 (m, 2H), 3.13 (s, 2H), 3.08 (s, 2H), 2.67 (s, 6H), 2.47 (s, 3H).

### Procedure for the Synthesis of 318

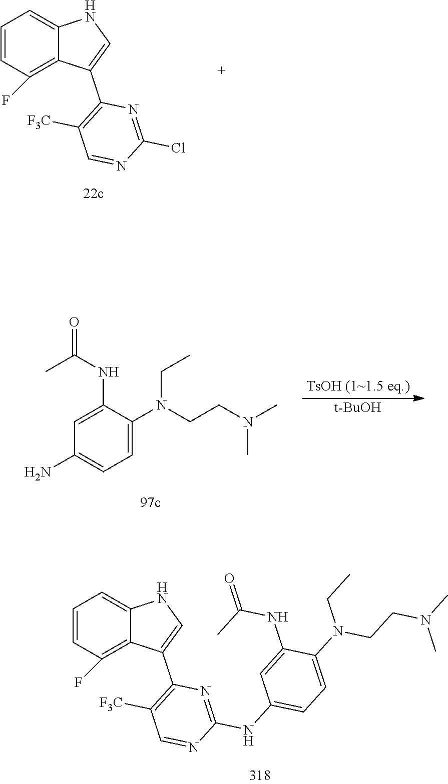

### N-(2-((2-(dimethylamino)ethyl)(ethyl)amino)-5-((4-(4-fluoro-1H-indol-3-yl)-5-(trifluoromethyl)pyrimidin-2-yl)amino)phenyl)acetamide (318)

Synthesis of 318 was similar to that of 296. The residue was purified by preparative thin layer chromatography (dichloromethane/methanol=12/1) to give the title product 318 (30 mg, yield 14.5%) as a gray solid. ¹H NMR (500 MHz, DMSO-d₆) δ 12.05 (s, 1H), 10.15 (s, 1H), 9.92 (s, 1H), 8.78 (s, 1H), 8.36 (s, 1H), 7.64 (s, 1H), 7.59 (s, 1H), 7.34 (d, J=8.2 Hz, 1H), 7.16 (td, J=8.0, 5.0 Hz, 2H), 6.84 (dd, J=11.1, 7.8 Hz, 1H), 3.00 (s, 2H), 2.91 (q, J=6.8 Hz, 2H), 2.51 (s, 2H), 2.37 (s, 6H), 2.11 (s, 3H), 0.83 (t, J=7.0 Hz, 3H).

### Procedure for the Synthesis of 319

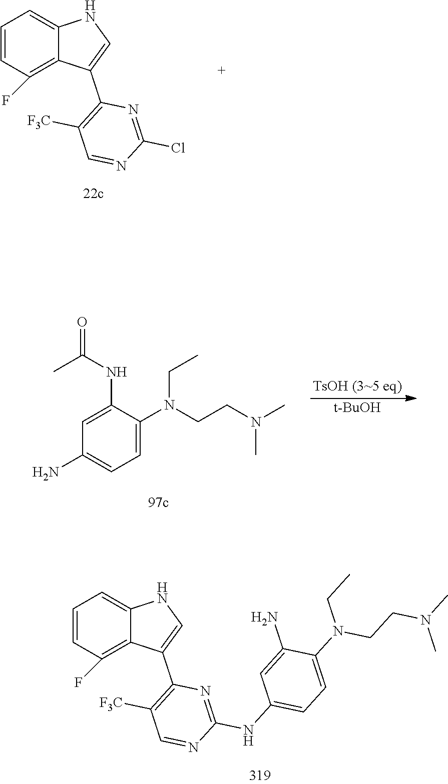

### N¹-(2-(dimethylamino)ethyl)-N¹-ethyl-N⁴-(4-(4-fluoro-1H-indol-3-yl)-5-(trifluoromethyl)pyrimidin-2-yl)benzene-1,2,4-triamine (319)

Synthesis of 319 was similar to that of 297. The residue was purified by preparative thin layer chromatography (dichloromethane/methanol=12/1) to give the title product 319 (51 mg, yield 21.4%) as a gray solid. ¹H NMR (500 MHz, DMSO-d₆) δ 11.97 (s, 1H), 9.92 (s, 1H), 8.75 (s, 1H), 7.62 (s, 1H), 7.34 (d, J=8.2 Hz, 1H), 7.16 (q, J=7.9 Hz, 1H), 7.01 (s, 2H), 6.94-6.79 (m, 2H), 3.10 (s, 2H), 2.92 (s, 2H), 2.87 (d, J=6.9 Hz, 2H), 2.61 (s, 6H), 0.88 (t, J=6.8 Hz, 3H).

### Procedure for the Synthesis of 320

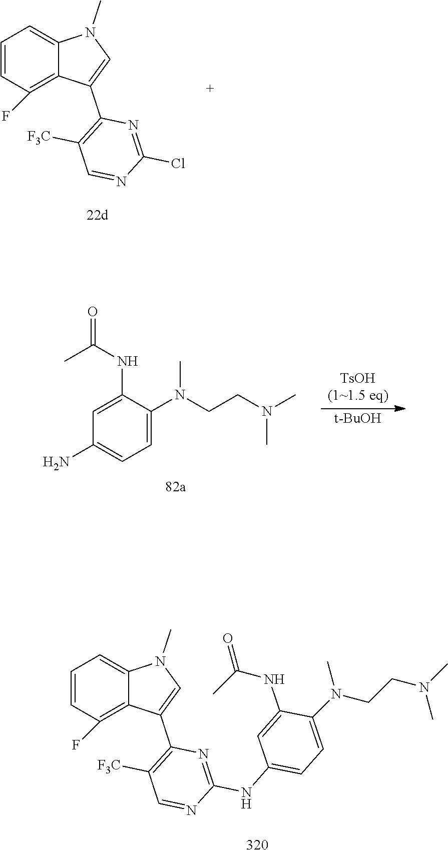

### N-(2-((2-(dimethylamino)ethyl)(methyl)amino)-5-((4-(4-fluoro-1-methyl-1H-indol-3-yl)-5-(trifluoromethyl)pyrimidin-2-yl)amino)phenyl)acetamide (320)

Synthesis of 320 was similar to that of 296. The residue was purified by preparative thin layer chromatography (dichloromethane/methanol=12/1) to give the title product 320 (101 mg, yield 40.8%) as a gray solid. MS-ESI (M+H)⁺: 544.6. ¹H NMR (500 MHz, DMSO-d₆) δ 10.13 (s, 1H), 9.80 (s, 1H), 8.78 (s, 1H), 8.38 (s, 1H), 7.71 (s, 1H), 7.53 (d, J=8.7 Hz, 1H), 7.39 (d, J=8.3 Hz, 1H), 7.24 (td, J=8.1, 4.9 Hz, 1H), 7.14 (d, J=8.7 Hz, 1H), 6.90 (dd, J=11.1, 7.8 Hz, 1H), 3.90 (s, 3H), 2.93 (s, 2H), 2.64 (s, 2H), 2.58 (s, 3H), 2.42 (s, 6H), 2.13 (s, 3H).

### Procedure for the Synthesis of 321

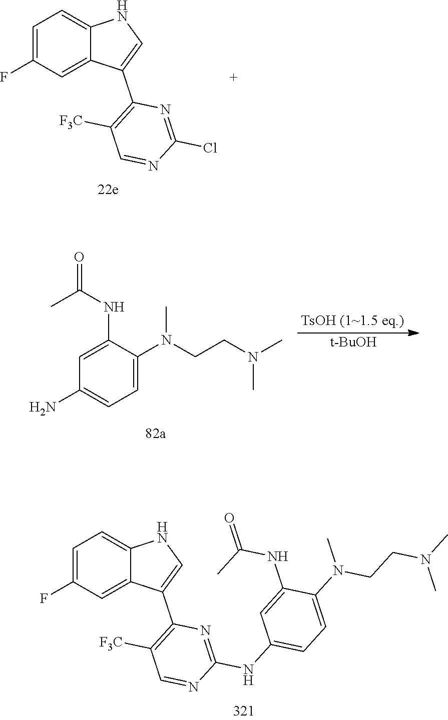 [SMILES: Fc1ccc2[nH]cc(-c3nc(Cl)ncc3C(F)(F)F)c2c1 ; "t-BuOH" ; CC(=O)Nc1cc(N)ccc1N(C)CCN(C)C ; CC(=O)Nc1cc(Nc2ncc(C(F)(F)F)c(-c3c[nH]c4ccc(F)cc34)n2)ccc1N(C)CCN(C)C]

### N-(2-((2-(dimethylamino)ethyl)(methyl)amino)-5-((4-(5-fluoro-1H-indol-3-yl)-5-(trifluoromethyl)pyrimidin-2-yl)amino)phenyl)acetamide (321)

Synthesis of 321 was similar to that of 296. The residue was purified by preparative thin layer chromatography (dichloromethane/methanol=12/1) to give the title product 321 (123 mg, yield 36.7%) as a gray solid. MS-ESI (M+H)⁺: 530.5. ¹H NMR (500 MHz, DMSO-d₆) δ 11.96 (s, 1H), 10.03 (d, J=7.8 Hz, 2H), 8.69 (s, 1H), 8.45 (s, 1H), 7.99 (s, 1H), 7.55-7.45 (m, 2H), 7.23 (d, J=8.5 Hz, 1H), 7.05 (t, J=8.7 Hz, 1H), 2.80 (s, 2H), 2.66 (s, 3H), 2.30 (s, 2H), 2.23 (s, 6H), 2.06 (s, 3H).

### Procedure for the Synthesis of 322

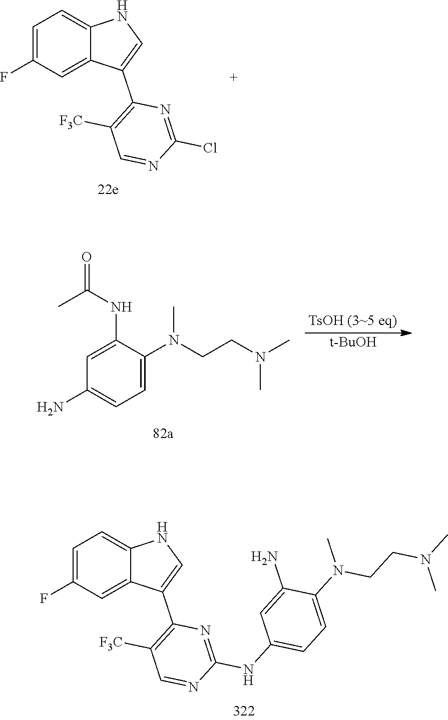 [SMILES: Fc1ccc2[nH]cc(-c3nc(Cl)ncc3C(F)(F)F)c2c1 ; "t-BuOH" ; CC(=O)Nc1cc(N)ccc1N(C)CCN(C)C ; CN(C)CCN(C)c1ccc(Nc2ncc(C(F)(F)F)c(-c3c[nH]c4ccc(F)cc34)n2)cc1N]

### N¹-(2-(dimethylamino)ethyl)-N⁴-(4-(5-fluoro-1H-indol-3-yl)-5-(trifluoromethyl)pyrimidin-2-yl)-N¹-methylbenzene-1,2,4-triamine (322)

Synthesis of 322 was similar to that of 297. The residue was purified by preparative thin layer chromatography (dichloromethane/methanol=12/1) to give the title product 322 (51 mg, yield 15.7%) as a gray solid. ¹H NMR (500 MHz, DMSO-d₆) δ 12.06 (s, 1H), 9.80 (s, 1H), 8.67 (s, 1H), 8.13 (s, 1H), 7.97 (s, 1H), 7.51 (dd, J=8.8, 4.7 Hz, 1H), 7.06 (td, J=9.0, 2.3 Hz, 1H), 7.00-6.90 (m, 3H), 3.25 (t, J=5.5 Hz, 2H), 3.14 (t, J=5.7 Hz, 2H), 2.75 (s, 6H), 2.51 (s, 3H).

### Procedure for the Synthesis of 323

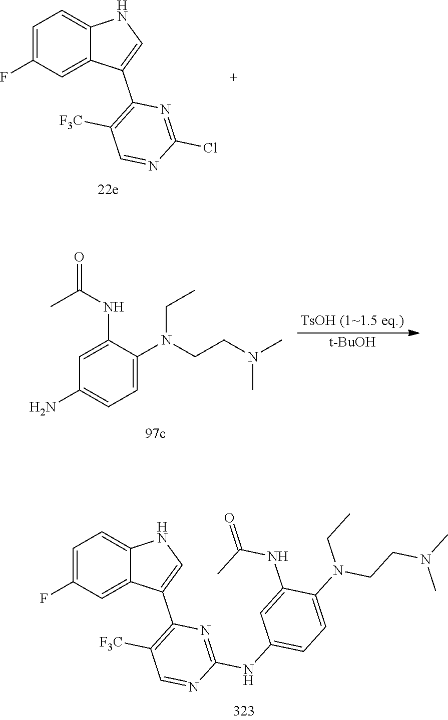 [SMILES: Fc1ccc2[nH]cc(-c3nc(Cl)ncc3C(F)(F)F)c2c1 ; "t-BuOH" ; CCN(CCN(C)C)c1ccc(N)cc1NC(C)=O ; CCN(CCN(C)C)c1ccc(Nc2ncc(C(F)(F)F)c(-c3c[nH]c4ccc(F)cc34)n2)cc1NC(C)=O]

### N-(2-((2-(dimethylamino)ethyl)(ethyl)amino)-5-((4-(5-fluoro-1H-indol-3-yl)-5-(trifluoromethyl)pyrimidin-2-yl)amino)phenyl)acetamide (323)

Synthesis of 323 was similar to that of 296. The residue was purified by preparative thin layer chromatography (dichloromethane/methanol=12/1) to give the title product 323 (23 mg, yield 8.9%) as a gray solid. ¹H NMR (500 MHz, DMSO-d₆) δ 12.01 (s, 1H), 10.07 (s, 1H), 9.95 (s, 1H), 8.71 (s, 1H), 8.39 (s, 1H), 8.15 (s, 1H), 7.99 (s, 1H), 7.57 (d, J=7.1 Hz, 1H), 7.50 (dd, J=8.8, 4.7 Hz, 1H), 7.23 (d, J=8.6 Hz, 1H), 7.06 (t, J=7.8 Hz, 1H), 3.01 (s, 2H), 2.96 (q, J=7.0 Hz, 2H), 2.36 (s, 6H), 2.09 (s, 3H), 0.86 (t, J=7.0 Hz, 3H).

### Procedure for the Synthesis of 324

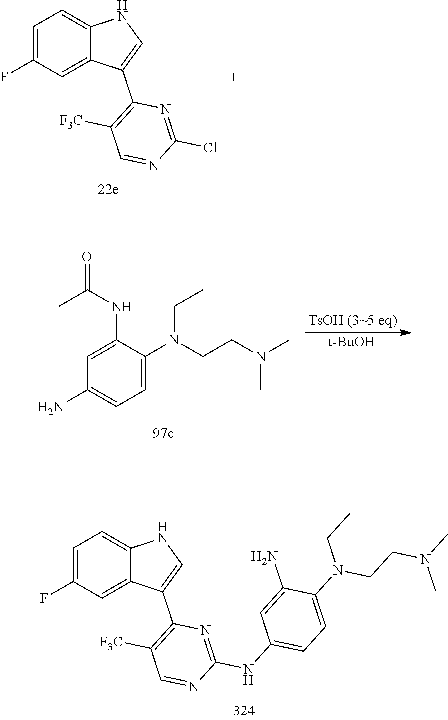 [SMILES: Fc1ccc2[nH]cc(-c3nc(Cl)ncc3C(F)(F)F)c2c1 ; "t-BuOH" ; CCN(CCN(C)C)c1ccc(N)cc1NC(C)=O ; CCN(CCN(C)C)c1ccc(Nc2ncc(C(F)(F)F)c(-c3c[nH]c4ccc(F)cc34)n2)cc1N]

### N¹-(2-(dimethylamino)ethyl)-N¹-ethyl-N⁴-(4-(5-fluoro-1H-indol-3-yl)-5-(trifluoromethyl)pyrimidin-2-yl)benzene-1,2,4-triamine (324)

Synthesis of 324 was similar to that of 297. The residue was purified by preparative thin layer chromatography (dichloromethane/methanol=12/1) to give the title product 324 (23 mg, yield 9.6%) as a gray solid. ¹H NMR (500 MHz, DMSO-d₆) δ 12.06 (s, 1H), 9.82 (s, 1H), 8.68 (s, 1H), 8.16 (s, 1H), 7.97 (s, 1H), 7.51 (dd, J=8.8, 4.7 Hz, 1H), 7.06 (td, J=9.0, 2.5 Hz, 1H), 6.99 (d, J=7.9 Hz, 2H), 6.95 (d, J=8.6 Hz, 1H), 3.19 (t, J=5.7 Hz, 2H), 3.12 (d, J=5.2 Hz, 2H), 2.91 (q, J=7.0 Hz, 2H), 2.75 (s, 6H), 0.91 (t, J=7.0 Hz, 3H).

### Procedure for the Synthesis of 325

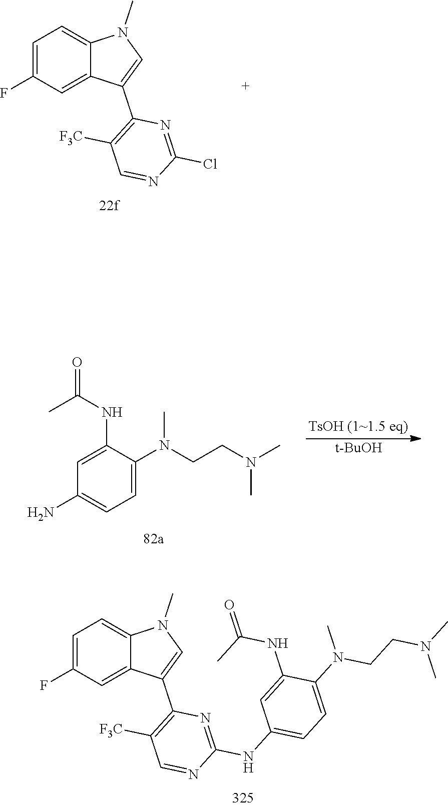

### N-(2-((2-(dimethylamino)ethyl)(methyl)amino)-5-((4-(5-fluoro-1-methyl-1H-indol-3-yl)-5-(trifluoromethyl)pyrimidin-2-yl)amino)phenyl)acetamide (325)

Synthesis of 325 was similar to that of 296. The residue was purified by flash column chromatography (dichloromethane/methanol=15/1) to give the title product 325 (41 mg, yield 16.6%) as a gray solid. LC-MS: m/z 544.4 [M+H]⁺. ¹H NMR (500 MHz, DMSO-d₆) δ 10.02 (s, 1H), 9.99 (s, 1H), 8.69 (s, 1H), 8.43 (s, 1H), 7.99 (s, 1H), 7.55 (dd, J=8.9, 4.6 Hz, 2H), 7.49 (d, J=9.0 Hz, 1H), 7.23 (d, J=8.6 Hz, 1H), 7.15-7.11 (m, 1H), 3.93 (s, 3H), 2.83 (s, 2H), 2.65 (s, 3H), 2.27 (s, 6H), 2.07 (s, 3H).

### Procedure for the Synthesis of 326

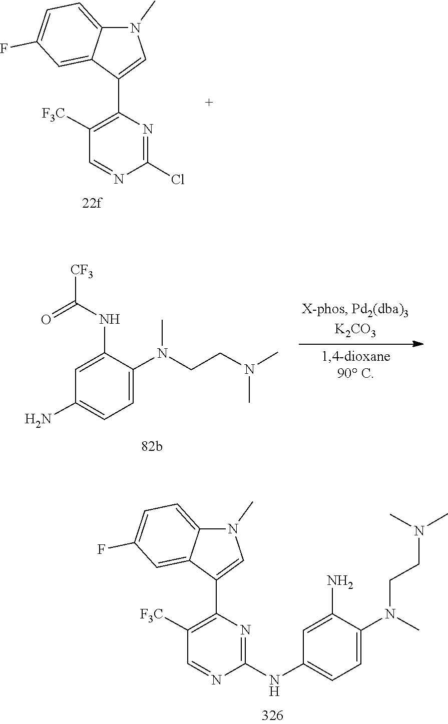 [SMILES: Cn1cc(-c2nc(Cl)ncc2C(F)(F)F)c2cc(F)ccc21 ; CN(C)CCN(C)c1ccc(N)cc1NC(=O)C(F)(F)F ; CN(C)CCN(C)c1ccc(Nc2ncc(C(F)(F)F)c(-c3cn(C)c4ccc(F)cc34)n2)cc1N]

### N¹-(2-(dimethylamino)ethyl)-N⁴-(4-(5-fluoro-1-methyl-1H-indol-3-yl)-5-(trifluoromethyl)pyrimidin-2-yl)-N¹-methylbenzene-1,2,4-triamine (326)

X-Phos (0.02 g, 0.04 mmol), Pd₂(dba)₃ (0.02 g, 0.02 mmol), K₂CO₃ (0.16 g, 1.16 mmol) and 82b (0.17 g, 0.55 mmol) was added to a solution of 22f (0.15 g, 0.45 mmol) in 1,4-dioxane (40 ml). The resulting mixture was stirred at 90° C. for 4 hrs. Then the reaction mixture was cooled down and filtered. The filtrate was concentrated. The residue was purified by flash column chromatography (dichloromethane/methanol=20/1) to give the title compound 326 (70 mg, yield 31.0%). ¹H NMR (500 MHz, DMSO-d6) δ 9.79 (s, 1H), 8.66 (s, 1H), 7.97 (s, 1H), 7.62-7.52 (m, 1H), 7.13 (t, J=10.3 Hz, 1H), 7.00-6.86 (m, 3H), 5.76 (s, 1H), 3.93 (s, 3H), 2.99 (s, 3H), 2.54 (s, 3H), 2.52-2.49 (m, 2H), 2.47 (s, 6H).

### Procedure for the Synthesis of 327

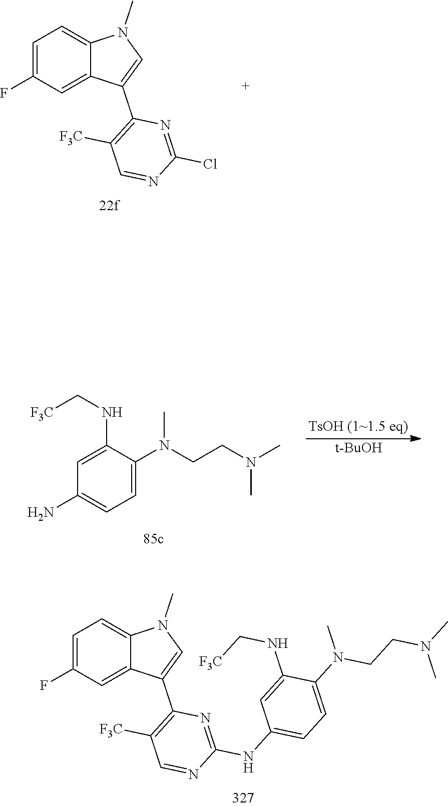

### N¹-(2-(dimethylamino)ethyl)-N⁴-(4-(5-fluoro-1-methyl-1H-indol-3-yl)-5-(trifluoromethyl)pyrimidin-2-yl)-N¹-methyl-N²-(2,2,2-trifluoroethyl)benzene-1,2,4-triamine (327)

Synthesis of 327 was similar to that of 296. The residue was purified by preparative thin layer chromatography (dichloromethane/methanol=12/1) to give the title product 327 (21 mg, yield 6.6%) as a gray solid. LC-MS: m/z 584.3 [M+H]⁺. ¹H NMR (500 MHz, DMSO-d₆) δ 9.89 (s, 1H), 8.69 (s, 1H), 8.13 (s, 1H), 7.98 (s, 1H), 7.57 (dd, J=9.0, 4.6 Hz, 1H), 7.14 (dt, J=9.4, 4.4 Hz, 2H), 7.11 (s, 1H), 7.02 (d, J=8.4 Hz, 1H), 6.52 (t, J=6.9 Hz, 1H), 3.93 (s, 3H), 3.80 (d, J=12.3 Hz, 2H), 3.16 (d, J=11.1 Hz, 2H), 3.10 (t, J=5.1 Hz, 2H), 2.64 (s, 6H).

### Procedure for the Syntheses of 328a and 328b

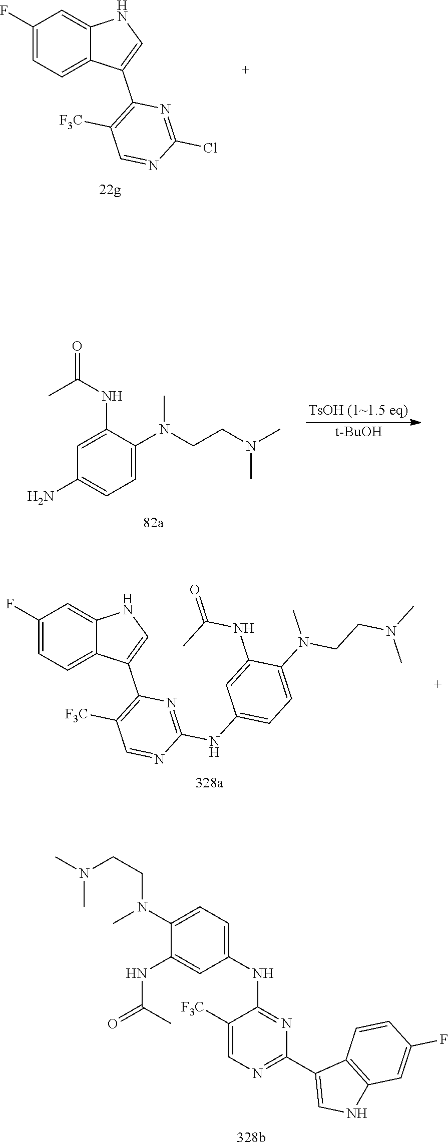

### N-(2-((2-(dimethylamino)ethyl)(methyl)amino)-5-((4-(6-fluoro-1H-indol-3-yl)-5-(trifluoromethyl)pyrimidin-2-yl)amino)phenyl)acetamide (328a) & N-(2-((2-(dimethylamino)ethyl)(methyl)amino)-5-((2-(6-fluoro-1H-indol-3-yl)-5-(trifluoromethyl)pyrimidin-4-yl)amino)phenyl)acetamide (328b)

Synthesis of 328a and 328b were similar to that of 296. The residue was purified by flash column chromatography (dichloromethane/methanol=15/1) to give the title product 328a (51 mg, yield 14.5%) as a gray solid and product 328b (33 mg, yield 9.4%) as a gray solid.

For 328a: MS-ESI (M+H)⁺: 530.5. ¹H NMR (500 MHz, DMSO-d₆) δ 11.94 (s, 1H), 10.04 (s, 1H), 9.66 (s, 1H), 8.71 (s, 1H), 8.33 (s, 2H), 7.90 (s, 1H), 7.54 (d, J=7.2 Hz, 1H), 7.29 (d, J=9.1 Hz, 1H), 7.20 (d, J=8.6 Hz, 1H), 6.96 (s, 1H), 3.20-2.89 (m, 4H), 2.62 (s, 6H), 2.59 (s, 3H), 2.18 (s, 3H);

For 328b: MS-ESI (M+H)⁺: 530.7. ¹H NMR (500 MHz, DMSO-d₆) δ 11.85 (s, 1H), 9.84 (s, 1H), 8.89 (s, 1H), 8.59 (s, 1H), 8.37 (s, 1H), 8.22 (d, J=2.8 Hz, 1H), 8.00 (dd, J=8.9, 5.7 Hz, 1H), 7.33 (d, J=8.6 Hz, 1H), 7.21 (d, J=4.8 Hz, 1H), 7.19 (t, J=3.0 Hz, 1H), 6.79 (t, J=9.2 Hz, 1H), 3.15 (d, J=21.0 Hz, 4H), 2.69 (s, 3H), 2.56 (s, 6H), 2.18 (s, 3H).

### Procedure for the Synthesis of 329

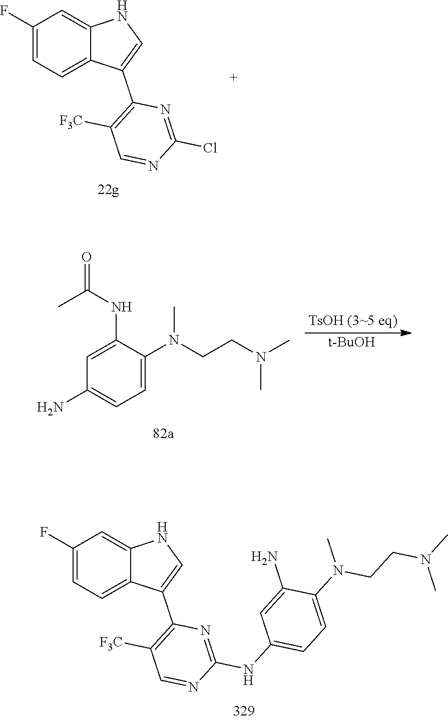 [SMILES: Fc1ccc2c(-c3nc(Cl)ncc3C(F)(F)F)c[nH]c2c1 ; "t-BuOH" ; CC(=O)Nc1cc(N)ccc1N(C)CCN(C)C ; CN(C)CCN(C)c1ccc(Nc2ncc(C(F)(F)F)c(-c3c[nH]c4cc(F)ccc34)n2)cc1N]

### N¹-(2-(dimethylamino)ethyl)-N⁴-(4-(6-fluoro-1H-indol-3-yl)-5-(trifluoromethyl)pyrimidin-2-yl)-N¹-methylbenzene-1,2,4-triamine (329)

Synthesis of 329 was similar to that of 297. The residue was purified by preparative thin layer chromatography (dichloromethane/methanol=12/1) to give the title product 329 (82 mg, yield 27.9%) as a gray solid. MS-ESI (M+H)⁺: 488.5. ¹H NMR (500 MHz, DMSO-d₆) δ 11.99 (s, 1H), 9.76 (s, 1H), 8.68 (s, 1H), 7.89 (s, 1H), 7.29 (d, J=9.6 Hz, 1H), 7.00 (s, 1H), 6.96 (d, J=9.0 Hz, 1H), 6.92 (s, 2H), 3.06 (s, 2H), 3.00 (s, 2H), 2.58 (s, 6H), 2.53 (s, 3H).

### Procedure for the Syntheses of 330a and 330b

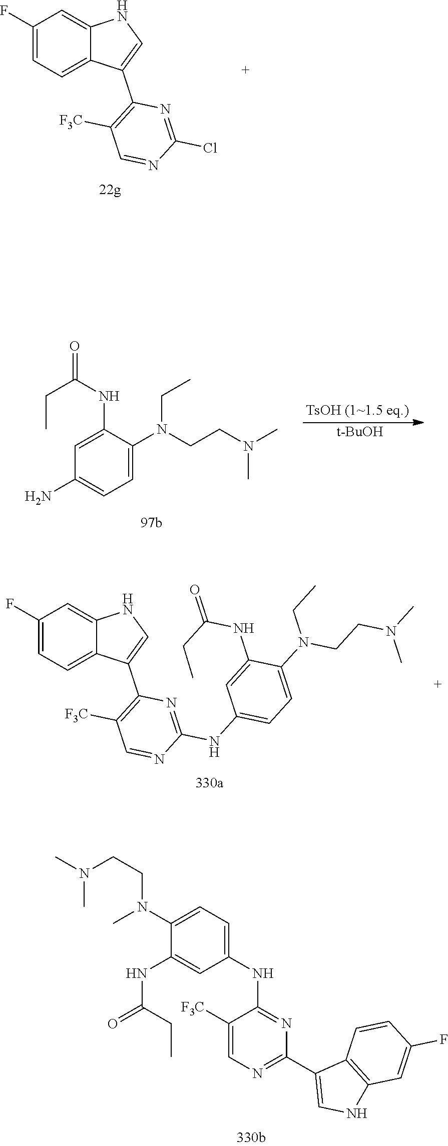

### N-(2-((2-(dimethylamino)ethyl)(ethyl)amino)-5-((4-(6-fluoro-1H-indol-3-yl)-5-(trifluoromethyl)pyrimidin-2-yl)amino)phenyl)propionamide (330a) & N-(2-((2-(dimethylamino)ethyl)(ethyl)amino)-5-((2-(6-fluoro-1H-indol-3-yl)-5-(trifluoromethyl)pyrimidin-4-yl)amino)phenyl)propionamide (330b)

Synthesis of 330a and 330b were similar to that of 296. The residue was purified by preparative thin layer chromatography (dichloromethane/methanol=12/1) to give the title product 330a (21 mg, yield 5.0%) as a gray solid and products 330b (21 mg, yield 5.0%) as a gray solid.

For 330a: MS-ESI (M+H)⁺: 558.6. ¹H NMR (500 MHz, DMSO-d₆) δ 11.91 (s, 1H), 10.04 (s, 1H), 9.87 (s, 1H), 8.72 (s, 1H), 8.41 (s, 1H), 8.35 (s, 1H), 7.91 (s, 1H), 7.53 (d, J=7.6 Hz, 1H), 7.29 (d, J=9.7 Hz, 1H), 7.24 (d, J=8.7 Hz, 1H), 6.91 (s, 1H), 3.03 (s, 2H), 2.95 (q, J=7.0 Hz, 2H), 2.39 (s, 2H), 2.38-2.26 (m, 6H), 1.11 (t, J=7.5 Hz, 3H), 0.87 (t, J=7.1 Hz, 3H);

For 330b: MS-ESI (M+H)⁺: 558.5. ¹H NMR (500 MHz, DMSO-d₆) δ 11.85 (s, 1H), 9.90 (s, 1H), 8.88 (s, 1H), 8.60 (s, 1H), 8.40 (s, 1H), 8.23 (d, J=2.8 Hz, 1H), 8.11-7.99 (m, 1H), 7.38 (d, J=8.5 Hz, 1H), 7.26-7.22 (m, 1H), 7.21 (dd, J=9.8, 2.4 Hz, 1H), 6.73 (t, J=8.8 Hz, 1H), 3.14 (s, 2H), 3.04 (q, J=7.0 Hz, 2H), 2.42 (s, 6H), 1.08 (t, J=7.5 Hz, 3H), 0.94 (t, J=7.0 Hz, 3H).

### Procedure for the Syntheses of 331a and 331b

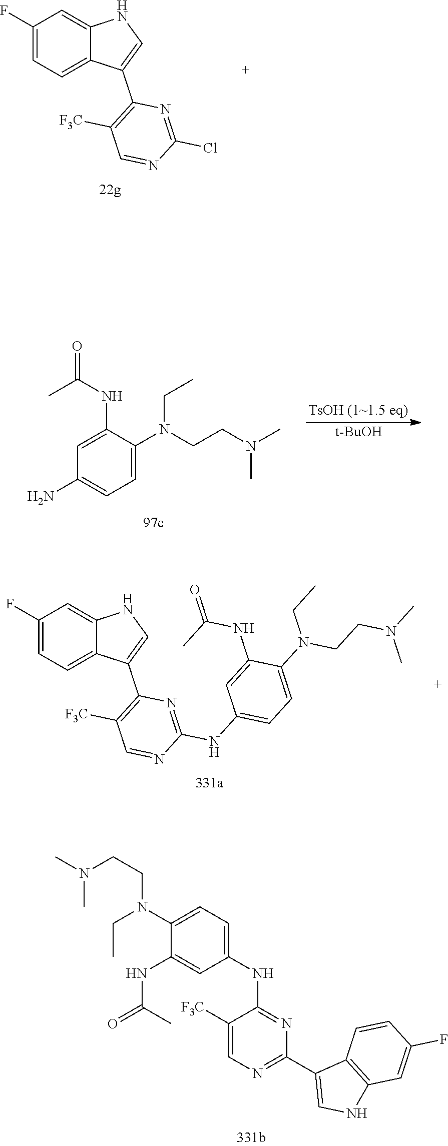

### N-(2-((2-(dimethylamino)ethyl)(ethyl)amino)-5-((4-(6-fluoro-1H-indol-3-yl)-5-(trifluoromethyl)pyrimidin-2-yl)amino)phenyl)acetamide (331a) & N-(2-((2-(dimethylamino)ethyl)(ethyl)amino)-5-((2-(6-fluoro-1H-indol-3-yl)-5-(trifluoromethyl)pyrimidin-4-yl)amino)phenyl)acetamide (331b)

Synthesis of 331a and 331b were similar to that of 296. The residue was purified by preparative thin layer chromatography (dichloromethane/methanol=12/1) to give the title product 331a (14 mg, yield 4.1%) as a gray solid and product 331b (18 mg, yield 5.2%) as a gray solid.

For 331a: MS-ESI (M+H)⁺: 544.4. ¹H NMR (500 MHz, DMSO-d₆) δ 11.94 (s, 1H), 10.04 (s, 1H), 9.89 (s, 1H), 8.72 (s, 1H), 8.35 (d, J=23.0 Hz, 2H), 7.91 (s, 1H), 7.54 (d, J=8.6 Hz, 1H), 7.29 (d, J=9.3 Hz, 1H), 7.23 (d, J=8.7 Hz, 1H), 6.93 (d, J=10.6 Hz, 1H), 3.06 (s, 2H), 2.96 (q, J=7.1 Hz, 2H), 2.61 (s, 2H), 2.11 (s, 3H), 0.87 (t, J=7.0 Hz, 3H).

For 331b: MS-ESI (M+H)⁺: 544.6. ¹H NMR (500 MHz, DMSO-d₆) δ 11.86 (d, J=3.0 Hz, 1H), 9.93 (s, 1H), 8.88 (s, 1H), 8.60 (s, 1H), 8.45-8.33 (m, 1H), 8.23 (d, J=2.9 Hz, 1H), 8.05 (dd, J=8.8, 5.7 Hz, 1H), 7.37 (d, J=8.5 Hz, 1H), 7.26-7.22 (m, 1H), 7.22-7.17 (m, 1H), 6.75 (s, 1H), 3.15 (s, 2H), 3.05 (q, J=7.0 Hz, 2H), 2.74 (s, 2H), 2.51-2.28 (m, 6H), 2.14 (s, 3H), 0.95 (t, J=7.0 Hz, 3H).

### Procedure for the Synthesis of 332

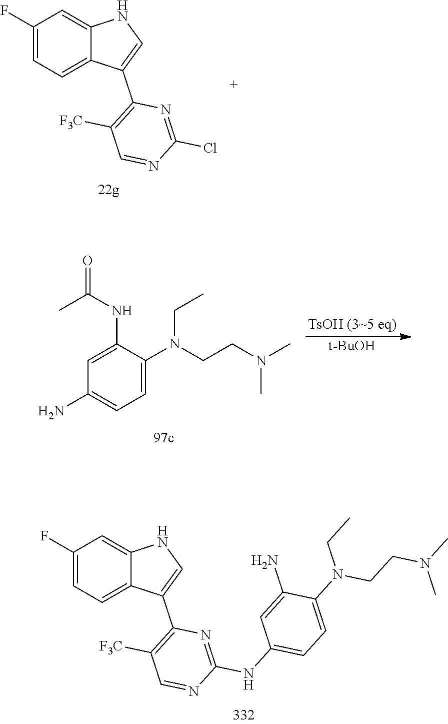

### N¹-(2-(dimethylamino)ethyl)-N¹-ethyl-N⁴-(4-(6-fluoro-1H-indol-3-yl)-5-(trifluoromethyl)pyrimidin-2-yl)benzene-1,2,4-triamine (332)

Synthesis of 332 was similar to that of 297. The residue was purified by preparative thin layer chromatography (dichloromethane/methanol=12/1) to give the title product 332 (51 mg, yield 20.1%) as a gray solid. MS-ESI (M+H)⁺: 502.6. ¹H NMR (500 MHz, DMSO-d₆) δ 11.99 (s, 1H), 9.79 (s, 1H), 8.69 (s, 1H), 8.36 (s, 1H), 7.89 (s, 1H), 7.29 (dd, J=9.7, 2.3 Hz, 1H), 7.02 (s, 1H), 6.95 (d, J=8.0 Hz, 3H), 3.16 (t, J=6.0 Hz, 2H), 3.02 (s, 2H), 2.91 (q, J=7.0 Hz, 2H), 2.68 (s, 6H), 0.91 (t, J=7.0 Hz, 3H).

### Procedure for the Synthesis of 333

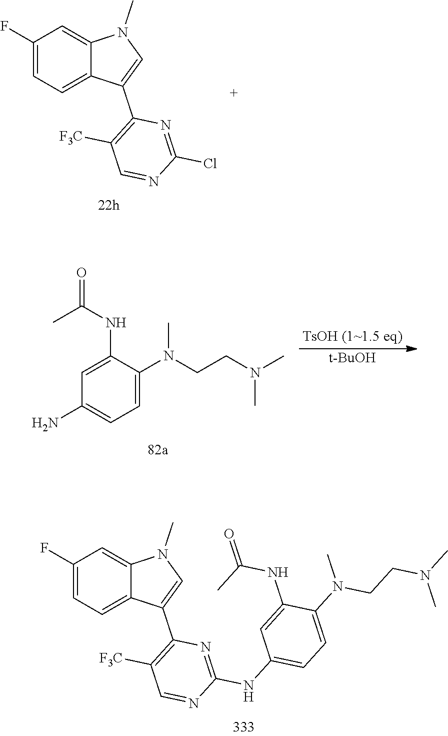 [SMILES: Cn1cc(-c2nc(Cl)ncc2C(F)(F)F)c2ccc(F)cc21 ; "t-BuOH" ; CC(=O)Nc1cc(N)ccc1N(C)CCN(C)C ; CC(=O)Nc1cc(Nc2ncc(C(F)(F)F)c(-c3cn(C)c4cc(F)ccc34)n2)ccc1N(C)CCN(C)C]

### N-(2-((2-(dimethylamino)ethyl)(methyl)amino)-5-((4-(6-fluoro-1-methyl-1H-indol-3-yl)-5-(trifluoromethyl)pyrimidin-2-yl)amino)phenyl)acetamide (333)

Synthesis of 333 was similar to that of 296. The residue was purified by preparative thin layer chromatography (dichloromethane/methanol=12/1) to give the title product 333 (36 mg, yield 14.6%) as a gray solid. MS-ESI (M+H)⁺: 544.6. ¹H NMR (500 MHz, DMSO-d₆) δ 10.53 (s, 1H), 10.05 (s, 1H), 9.71 (s, 1H), 8.71 (s, 1H), 8.35 (s, 1H), 7.91 (s, 1H), 7.53 (d, J=8.6 Hz, 1H), 7.44 (dd, J=9.9, 2.5 Hz, 1H), 7.19 (d, J=8.7 Hz, 1H), 7.01 (d, J=9.7 Hz, 1H), 3.89 (s, 3H), 3.11 (s, 2H), 2.63 (s, 2H), 2.60 (d, J=10.8 Hz, 6H), 2.19 (s, 3H).

### Procedure for the Synthesis of 334

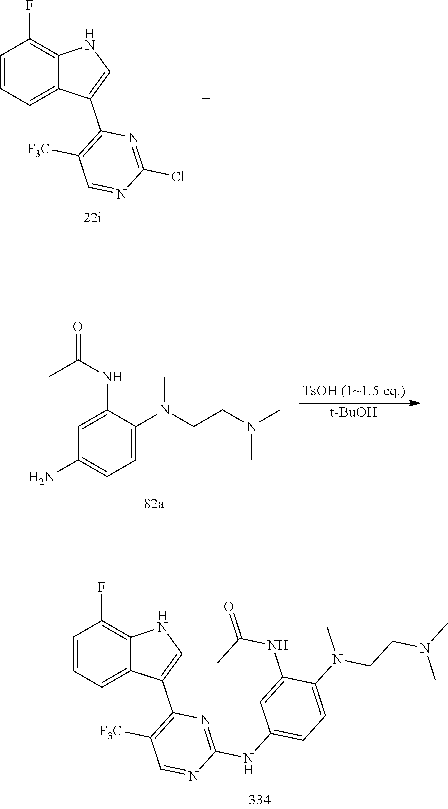

### N-(2-((2-(dimethylamino)ethyl)(methyl)amino)-5-((4-(7-fluoro-1H-indol-3-yl)-5-(trifluoromethyl)pyrimidin-2-yl)amino)phenyl)acetamide (334)

Synthesis of 334 was similar to that of 296. The residue was purified by preparative thin layer chromatography (dichloromethane/methanol=12/1) to give the title product 334 (38 mg, yield 10.8%) as a gray solid. MS-ESI (M+H)⁺: 530.3. ¹H NMR (500 MHz, DMSO-d₆) δ 12.46 (s, 1H), 10.07 (s, 1H), 9.78 (s, 1H), 8.74 (s, 1H), 8.36 (s, 1H), 8.12 (s, 1H), 7.86 (s, 1H), 7.53 (d, J=7.7 Hz, 1H), 7.19 (d, J=8.6 Hz, 1H), 7.13-7.01 (m, 2H), 3.03 (s, 2H), 2.85 (s, 2H), 2.59 (s, 4H), 2.50 (s, 6H), 2.17 (s, 3H).

### Procedure for the Synthesis of 335

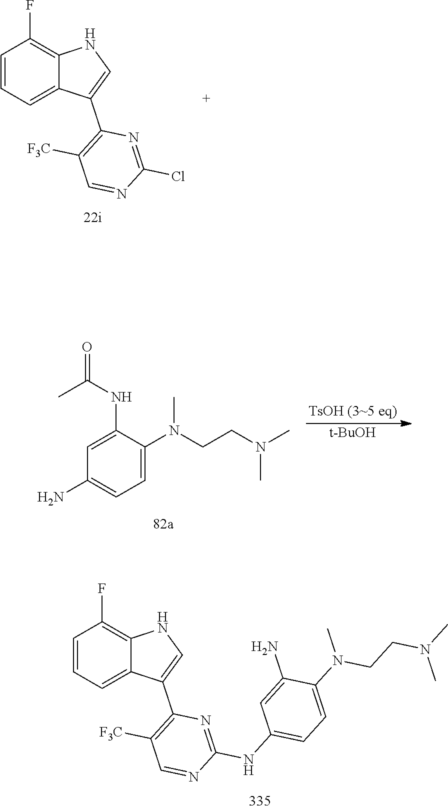

### N¹-(2-(dimethylamino)ethyl)-N⁴-(4-(7-fluoro-1H-indol-3-yl)-5-(trifluoromethyl)pyrimidin-2-yl)-N¹-methylbenzene-1,2,4-triamine (335)

Synthesis of 335 was similar to that of 297. The residue was purified by preparative thin layer chromatography (dichloromethane/methanol=12/1) to give the title product 335 (60 mg, yield 19.4%) as a light yellow solid. MS-ESI (M+H)⁺: 488.5. ¹H NMR (500 MHz, DMSO-d₆) δ 12.48 (s, 1H), 9.82 (s, 1H), 8.70 (s, 1H), 8.10 (s, 1H), 7.84 (s, 1H), 7.13-7.03 (m, 2H), 7.00 (s, 1H), 6.94 (d, J=8.5 Hz, 1H), 6.90 (d, J=8.5 Hz, 1H), 3.16-3.04 (m, 4H), 2.65 (s, 6H).

### Procedure for the Synthesis of 336

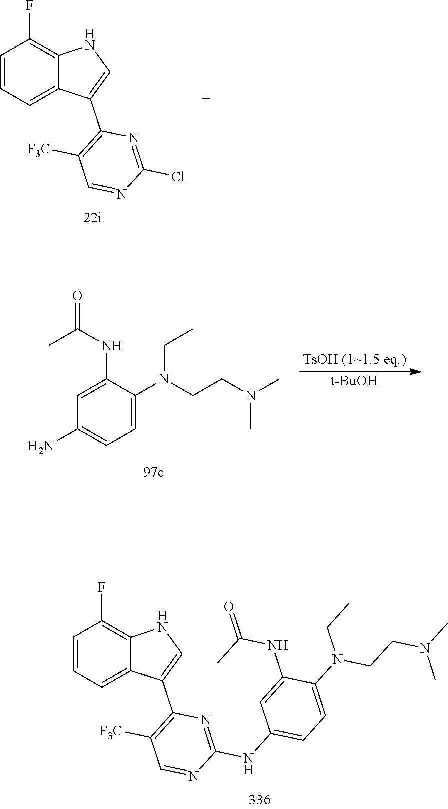

### N-(2-((2-(dimethylamino)ethyl)(ethyl)amino)-5-((4-(7-fluoro-1H-indol-3-yl)-5-(trifluoromethyl)pyrimidin-2-yl)amino)phenyl)acetamide (336)

Synthesis of 336 was similar to that of 296. The residue was purified by preparative thin layer chromatography (dichloromethane/methanol=12/1) to give the title product 336 (38 mg, yield 15.2%) as a yellow solid. ¹H NMR (500 MHz, DMSO-d₆) δ 12.54 (s, 1H), 10.08 (s, 1H), 9.74 (s, 1H), 8.74 (s, 1H), 8.32 (s, 1H), 8.11 (s, 1H), 7.85 (s, 1H), 7.55 (d, J=8.1 Hz, 1H), 7.18 (d, J=8.7 Hz, 1H), 7.12-7.03 (m, 2H), 3.19 (s, 2H), 2.94 (q, J=6.8 Hz, 3H), 2.61 (s, 6H), 2.20 (s, 3H), 0.89 (t, J=6.9 Hz, 3H).

### Procedure for the Synthesis of 337

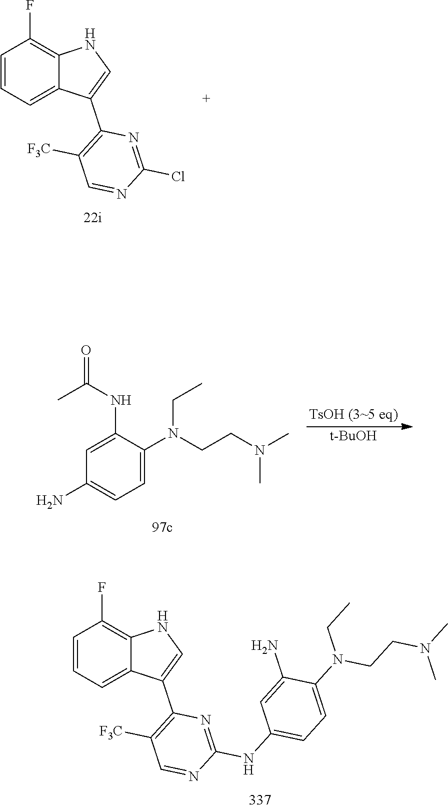

### N¹-(2-(dimethylamino)ethyl)-N¹-ethyl-N⁴-(4-(7-fluoro-1H-indol-3-yl)-5-(trifluoromethyl)pyrimidin-2-yl)benzene-1,2,4-triamine (337)

Synthesis of 337 was similar to that of 297. The residue was purified by preparative thin layer chromatography (dichloromethane/methanol=12/1) to give the title product 337 (36 mg, yield 16.2%) as a light yellow solid. MS-ESI (M+H)⁺: 502.6. ¹H NMR (500 MHz, DMSO-d₆) δ 12.47 (s, 1H), 9.83 (s, 1H), 8.71 (s, 1H), 8.10 (s, 1H), 7.85 (s, 1H), 7.07 (d, J=9.0 Hz, 2H), 7.04 (d, J=7.5 Hz, 1H), 6.96 (d, J=8.5 Hz, 1H), 6.93 (d, J=8.5 Hz, 1H), 3.13 (t, J=6.0 Hz, 2H), 2.93 (s, 2H), 2.89 (d, J=6.9 Hz, 2H), 2.61 (s, 6H), 0.91 (t, J=7.0 Hz, 3H).

### Procedure for the Syntheses of 339 and 340

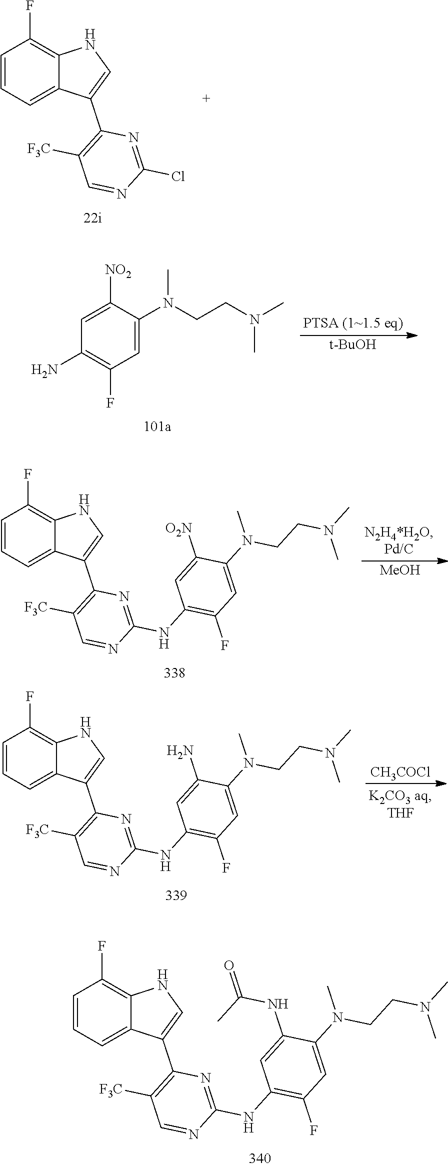

### N¹-(2-(dimethylamino)ethyl)-5-fluoro-N⁴-(4-(7-fluoro-1H-indol-3-yl)-5-(trifluoromethyl)pyrimidin-2-yl)-N¹-methyl-2-nitrobenzene-1,4-diamine (338)

Synthesis of 338 was similar to that of 296 by using 22i (436 mg, 1.38 mmol) and 101a (320 mg, 1.25 mmol) to afford the title compound 338 (338 mg, yield 50.6%).

### N¹-(2-(dimethylamino)ethyl)-5-fluoro-N⁴-(4-(7-fluoro-1H-indol-3-yl)-5-(trifluoromethyl)pyrimidin-2-yl)-N¹-methylbenzene-1,2,4-triamine (339)

A mixture of above compound 338 (338 mg, 0.63 mmol), Pd/C (5% on activated carbon, 143 mg, 0.07 mmol) and hydrazine hydrate (80%, 1 ml, 16 mmol) in methanol (25 ml) was heated at refluxing temperature for 4 hrs. After cooling to room temperature, the reaction mixtures was filtered through Celite® and the filtrates were concentrated to remove methanol. The residual was extracted with ethyl acetate. The organic layer was separated, washed with water, dried over Na₂SO₄, filtered and concentrated to afford the crude title compound 339 (360 mg). Part of this crude 339 (85 mg) was purified by preparative thin layer chromatography to afford the title compound 339 (26 mg, yield 34.5%) as a brown solid. The rest was directly used in the next step without further purification. LC-MS: m/z 506.5 [M+H]⁺.

### N-(2-((2-(dimethylamino)ethyl)(methyl)amino)-4-fluoro-5-((4-(7-fluoro-1H-indol-3-yl)-5-(trifluoromethyl)pyrimidin-2-yl)amino)phenyl)acetamide (340)

Acetyl chloride (80 mg, 1.02 mmol) was dropped to the mixture of 339 (270 mg, 0.53 mmol) and saturated K₂CO₃ aqueous (0.5 ml) in tetrahydrofuran (5 mL) at 0° C. The mixture was stirred for 1 hr. Then the mixture was quenched with H₂O and extracted with ethyl acetate. The organic layer was separated, dried over Na₂SO₄, and then concentrated under reduced pressure. The residue was purified by preparative thin layer chromatography (dichloromethane/methanol=8/1) to give the title compound 340 (13 mg, yield 5.0% over two steps) as an off-white solid. LC-MS: m/z 548.5 [M+H]⁺.

### Procedure for the Synthesis of 341

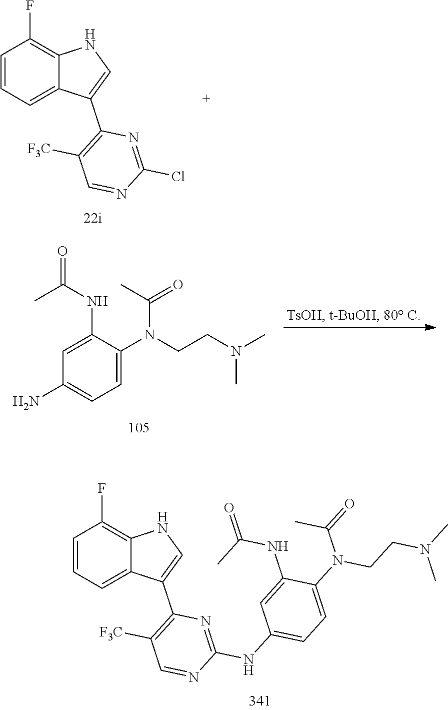 [SMILES: Fc1cccc2c(-c3nc(Cl)ncc3C(F)(F)F)c[nH]c12 ; CC(=O)Nc1cc(N)ccc1N(CCN(C)C)C(C)=O ; CC(=O)Nc1cc(Nc2ncc(C(F)(F)F)c(-c3c[nH]c4c(F)cccc34)n2)ccc1N(CCN(C)C)C(C)=O]

### N-(2-acetamido-4-((4-(7-fluoro-1H-indol-3-yl)-5-(trifluoromethyl)pyrimidin-2-yl)amino)phenyl)-N-(2-(dimethylamino)ethyl)acetamide (341)

To the mixture of 105 (186 mg, 0.67 mmol) and 22i (210 mg, 0.67 mmol) in t-BuOH (10 mL) was added TsOH (126 mg, 0.66 mmol). The mixture was stirred at 80° C. for 16 h. The mixture was concentrated and the residue was purified by preparative thin layer chromatography. The product was basified by aqueous K₂CO₃ to give 341 (13.3 mg, 3.6% yield) as off-white solid. MS-ESI (M+H⁺): 558.3; ¹H NMR (500 MHz, DMSO-d₆) δ 12.42 (s, 1H), 10.80 (s, 1H), 10.28 (s, 1H), 8.81 (s, 1H), 8.30 (s, 1H), 8.09 (s, 1H), 7.86 (s, 1H), 7.73 (dd, J=8.6, 2.1 Hz, 1H), 7.23 (d, J=8.6 Hz, 1H), 7.13-7.01 (m, 2H), 4.51 (t, J=9.2 Hz, 1H), 3.08 (dd, J=10.6, 3.8 Hz, 1H), 2.36 (dd, J=8.5, 4.2 Hz, 1H), 2.30 (dd, J=9.5, 4.0 Hz, 1H), 2.23 (s, 6H), 2.00 (s, 3H), 1.59 (s, 3H).

### Procedure for the Synthesis of 342

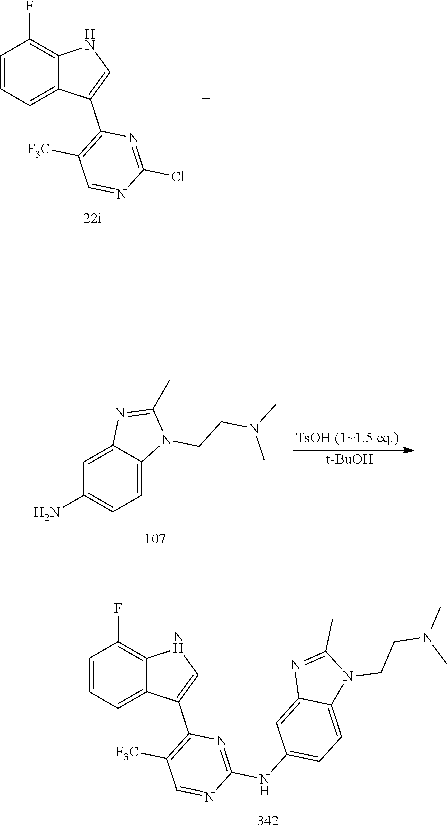

### 1-(2-(dimethylamino)ethyl)-N-(4-(7-fluoro-1H-indol-3-yl)-5-(trifluoromethyl)pyrimidin-2-yl)-2-methyl-1H-benzo[d]imidazol-5-amine (342)

Synthesis of 342 was similar to that of 296. The residue was purified by preparative thin layer chromatography (dichloromethane/methanol=12/1) to give the title product 342 (12 mg, yield 3.8%) as a light yellow solid. MS-ESI (M+H)⁺: 498.5. ¹H NMR (500 MHz, DMSO-d₆) δ 12.43 (s, 1H), 10.03 (s, 1H), 8.75 (s, 1H), 7.93 (s, 1H), 7.85 (s, 1H), 7.50 (dd, J=8.7, 1.9 Hz, 1H), 7.41 (d, J=8.6 Hz, 1H), 7.15-6.96 (m, 2H), 4.23 (t, J=6.5 Hz, 2H), 2.55 (t, J=6.5 Hz, 2H), 2.53 (s, 3H), 2.20 (s, 6H).

### Procedure for the Synthesis of 343

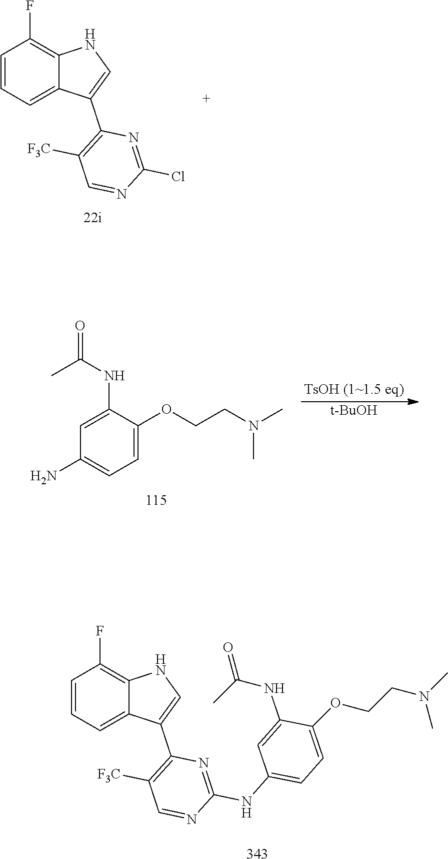

### N-(2-(2-(dimethylamino)ethoxy)-5-((4-(7-fluoro-1H-indol-3-yl)-5-(trifluoromethyl)pyrimidin-2-yl)amino)phenyl)acetamide (343)

Synthesis of 343 was similar to that of 296. The residue was purified by preparative thin layer chromatography (dichloromethane/methanol=12/1) to give the title product 343 (43 mg, yield 12.5%) as a rice white solid. MS-ESI (M+H)⁺: 517.6. ¹H NMR (500 MHz, DMSO-d₆) δ 12.41 (s, 1H), 10.01 (s, 1H), 9.63 (s, 1H), 8.73 (s, 1H), 8.29 (s, 1H), 7.85 (s, 1H), 7.48 (d, J=7.0 Hz, 1H), 7.05 (t, J=9.2 Hz, 3H), 4.13 (t, J=5.4 Hz, 2H), 2.73 (s, 2H), 2.37 (s, 6H), 2.08 (s, 3H).

### Procedure for the Synthesis of R-344

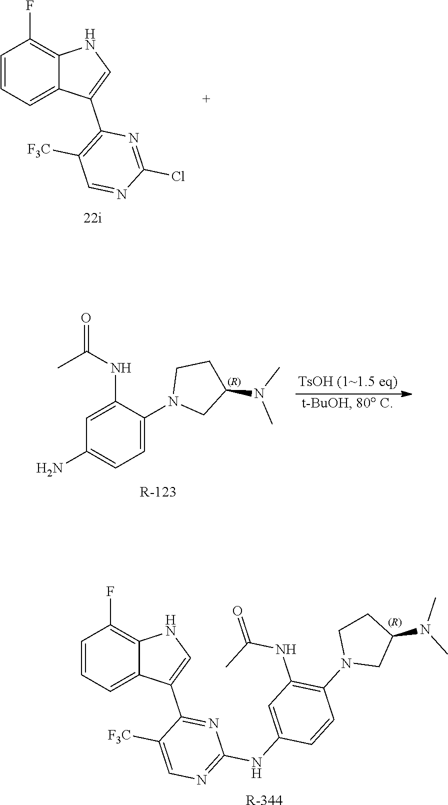

### (R)—N-(2-(3-(dimethylamino)pyrrolidin-1-yl)-5-((4-(7-fluoro-1H-indol-3-yl)-5-(tri-fluoromethyl)pyrimidin-2-yl)amino)phenyl)acetamide (R-344)

To the mixture of 22i (206 mg, 0.65 mmol) and R-123 (169 mg, 0.65 mmol) in t-BuOH (10 mL) was added TsOH (100 mg, 0.52 mmol). The mixture was stirred at 80° C. for 16 h. The mixture was concentrated and the residue was purified by preparative thin layer chromatography. The product was basified by aqueous K₂CO₃ to give R-344 (56 mg, 16% yield) as off-white solid. MS-ESI (M+H⁺): 542.7; ¹H NMR (500 MHz, DMSO-d₆) δ 12.44 (s, 1H), 9.99 (s, 1H), 9.24 (s, 1H), 8.73 (s, 1H), 8.10 (s, 1H), 7.89 (s, 1H), 7.85 (s, 1H), 7.51 (d, J=7.7 Hz, 1H), 7.19-7.00 (m, 2H), 6.91 (d, J=7.3 Hz, 1H), 3.28-3.02 (m, 5H), 2.49 (s, 6H), 2.22-2.14 (m, 1H), 2.08 (s, 3H), 2.03-1.93 (m, 1H).

### Procedure for the Synthesis of R-345

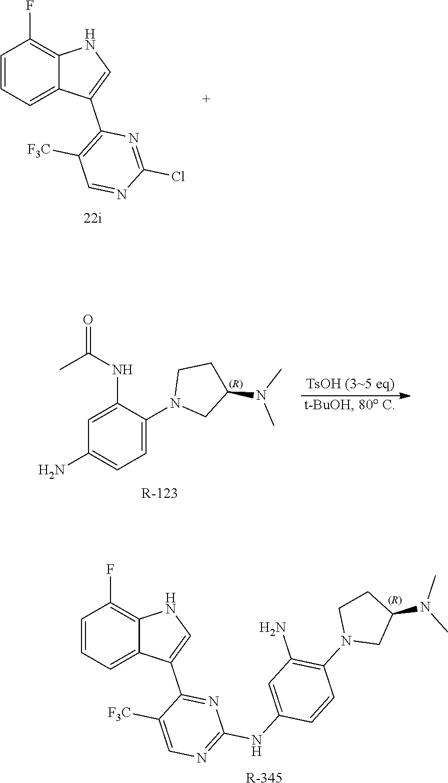

### (R)-4-(3-(dimethylamino)pyrrolidin-1-yl)-N¹-(4-(7-fluoro-1H-indol-3-yl)-5-(trifluoromethyl)pyrimidin-2-yl)benzene-1,3-diamine (R-345)

To the mixture of 22i (150 mg, 0.47 mmol) and R-123 (130 mg, 0.49 mmol) in t-BuOH (10 mL) was added TsOH (375 mg, 1.97 mmol). The mixture was stirred at 80° C. for 24 h. The mixture was concentrated and the residue was purified by preparative thin layer chromatography. The product was basified by aqueous K₂CO₃ to give R-345 (11.1 mg, 4.72% yield) as off-white solid. MS-ESI (M+H⁺): 500.4; ¹H NMR (500 MHz, DMSO-d₆) δ 12.41 (s, 1H), 9.78 (s, 1H), 8.70 (s, 1H), 8.10 (s, 1H), 7.85 (s, 1H), 7.17-6.99 (m, 3H), 6.94 (d, J=8.3 Hz, 1H), 6.85 (d, J=8.4 Hz, 1H), 4.76 (s, 2H), 3.09 (m, 4H), 2.98-2.91 (m, 1H), 2.50 (s, 6H), 2.21-2.09 (m, 1H), 1.96 (m, 1H).

### Procedure for the Synthesis of S-344

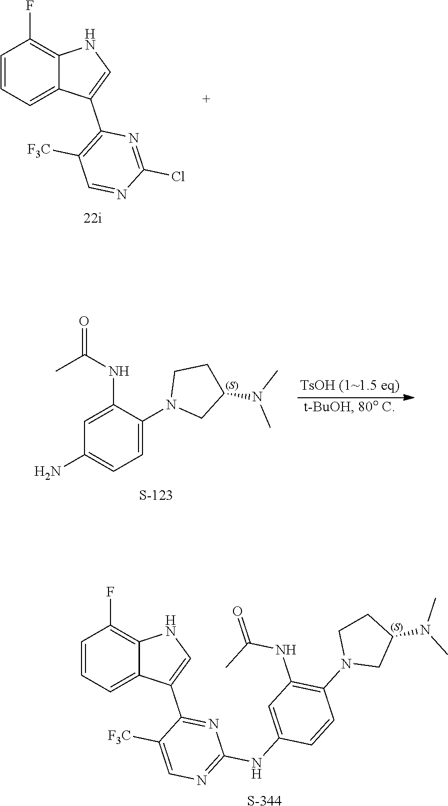

### (S)—N-(2-(3-(dimethylamino)pyrrolidin-1-yl)-5-((4-(7-fluoro-1H-indol-3-yl)-5-(tri-fluoromethyl)pyrimidin-2-yl)amino)phenyl)acetamide (S-344)

To the mixture of 22i (203 mg, 0.64 mmol) and S-123 (170 mg, 0.64 mmol) in t-BuOH (10 mL) was added TsOH (116 mg, 0.61 mmol). The mixture was stirred at 80° C. for 16 h. The mixture was concentrated and the residue was purified by preparative thin layer chromatography. The product was basified by aqueous NaHCO₃ to give S-344 (19 mg, 5.5% yield) as off-white solid. MS-ESI (M+H⁺): 542.5; ¹H NMR (500 MHz, DMSO-d₆) δ 12.42 (s, 1H), 9.99 (s, 1H), 9.22 (s, 1H), 8.73 (s, 1H), 8.09 (s, 1H), 7.88 (s, 1H), 7.85 (s, 1H), 7.51 (d, J=7.9 Hz, 1H), 7.15-7.02 (m, 2H), 6.92 (s, 1H), 3.28-3.02 (m, 5H), 2.51 (s, 6H), 2.23-2.15 (m, 1H), 2.07 (s, 3H), 1.99 (d, J=20.5 Hz, 1H).

### Procedure for the Synthesis of S-345

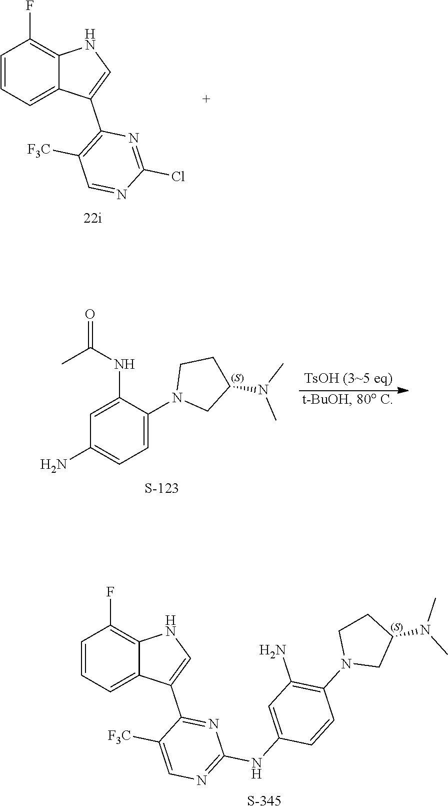

### (S)-4-(3-(dimethylamino)pyrrolidin-1-yl)-N¹-(4-(7-fluoro-1H-indol-3-yl)-5-(trifluoromethyl)pyrimidin-2-yl)benzene-1,3-diamine

To the mixture of 22i (150 mg, 0.48 mmol) and S-123 (125 mg, 0.48 mmol) in t-BuOH (10 mL) was added TsOH (360 mg, 1.89 mmol). The mixture was stirred at 80° C. for 16 h. The mixture was concentrated and the residue was purified by preparative thin layer chromatography. The product was basified by aqueous K₂CO₃ to give S-345 (31.4 mg, 0.063 mmol, 13% yield) as off-white solid. MS-ESI (M+H⁺): 500.4; ¹H NMR (500 MHz, Chloroform-d) δ 9.07 (s, 1H), 8.66 (s, 1H), 8.05 (d, J=8.1 Hz, 1H), 7.82 (d, J=2.1 Hz, 1H), 7.45 (s, 1H), 7.25-7.18 (m, 1H), 7.13 (m, 1H), 6.98 (m, 1H), 6.90 (d, J=8.4 Hz, 1H), 6.76 (m, 1H), 4.09 (s, 2H), 3.14 (m, 1H), 3.04-2.92 (m, 1H), 2.65 (s, 6H), 2.40-2.16 (m, 3H), 2.01 (m, 1H), 1.68-1.57 (m, 1H).

### Procedure for the Synthesis of 346

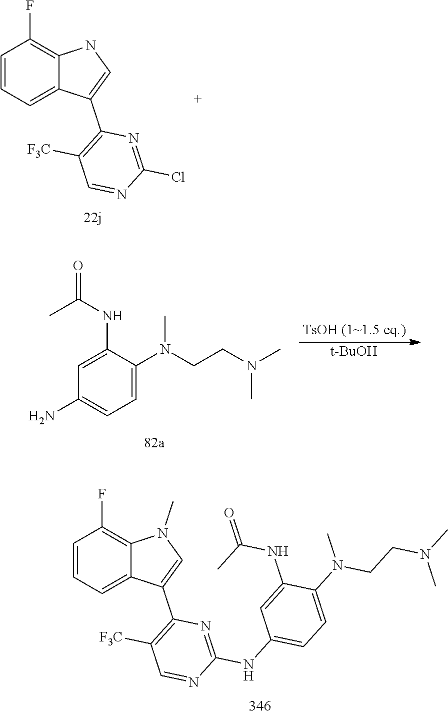

### N-(2-((2-(dimethylamino)ethyl)(methyl)amino)-5-((4-(7-fluoro-1-methyl-1H-indol-3-yl)-5-(trifluoromethyl)pyrimidin-2-yl)amino)phenyl)acetamide (346)

Synthesis of 346 was similar to that of 926. The residue was purified by preparative thin layer chromatography (dichloromethane/methanol=12/1) to give the title product 346 (96 mg, yield 29.1%) as a gray solid. ¹H NMR (500 MHz, DMSO-d₆) δ 10.05 (s, 1H), 9.93 (s, 1H), 8.72 (s, 1H), 8.39 (s, 1H), 8.09 (s, 1H), 7.86 (s, 1H), 7.50 (d, J=7.6 Hz, 1H), 7.18 (d, J=8.5 Hz, 1H), 7.06 (d, J=8.0 Hz, 2H), 4.08 (s, 3H), 2.91 (s, 2H), 2.61 (s, 3H), 2.57 (s, 1H), 2.36 (s, 6H), 2.12 (s, 3H).

### Procedure for the Synthesis of 347

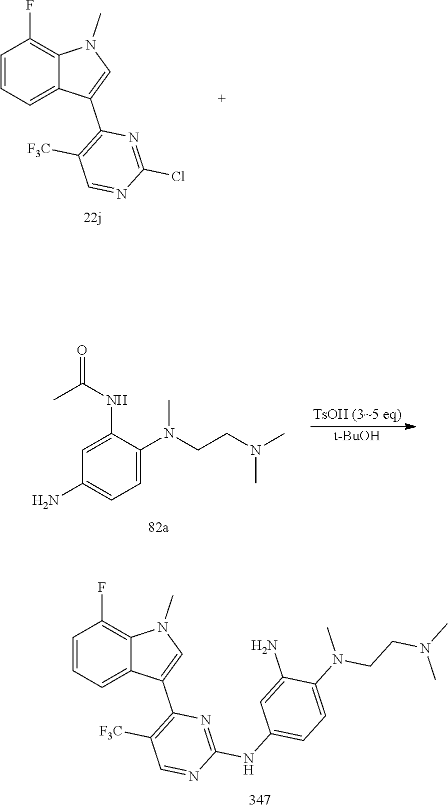

### N¹-(2-(dimethylamino)ethyl)-N⁴-(4-(7-fluoro-1-methyl-1H-indol-3-yl)-5-(trifluoromethyl)pyrimidin-2-yl)-N¹-methylbenzene-1,2,4-triamine (347)

Synthesis of 347 was similar to that of 927. The residue was purified by preparative thin layer chromatography (dichloromethane/methanol=12/1) to give the title product 347 (64 mg, yield 20.0%) as a gray solid. ¹H NMR (500 MHz, DMSO-d₆) δ 9.81 (s, 1H), 8.70 (s, 1H), 8.10 (s, 1H), 7.84 (s, 1H), 7.12-7.02 (m, 2H), 7.00 (s, 1H), 6.95-6.91 (m, 1H), 6.88 (d, J=8.3 Hz, 1H), 4.07 (s, 3H), 2.99 (s, 2H), 2.83 (s, 2H), 2.53 (s, 3H), 2.48 (s, 6H).

### Procedure for the Synthesis of 348

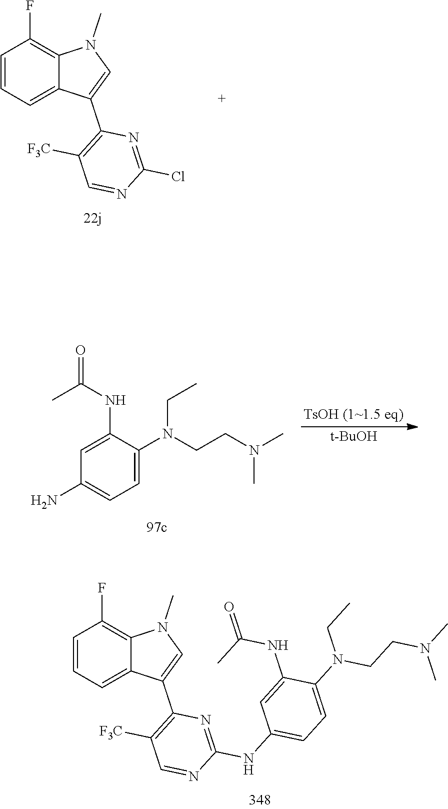

### N-(2-((2-(dimethylamino)ethyl)(ethyl)amino)-5-((4-(7-fluoro-1-methyl-1H-indol-3-yl)-5-(trifluoromethyl)pyrimidin-2-yl)amino)phenyl)acetamide (348)

Synthesis of 348 was similar to that of 296. The residue was purified by preparative thin layer chromatography (dichloromethane/methanol=12/1) to give the title product 348 (44 mg, yield 16.3%) as a gray solid. ¹H NMR (500 MHz, DMSO-d₆) δ 10.08 (s, 1H), 9.94 (s, 1H), 8.74 (s, 1H), 8.38 (s, 1H), 8.10 (s, 1H), 7.87 (s, 1H), 7.54 (d, J=6.9 Hz, 1H), 7.21 (d, J=8.4 Hz, 1H), 7.06 (d, J=8.3 Hz, 2H), 4.08 (s, 3H), 3.02 (s, 2H), 2.95 (q, J=6.7 Hz, 2H), 2.37 (s, 6H), 2.10 (s, 3H), 0.86 (t, J=6.8 Hz, 3H).

### Procedure for the Synthesis of 349

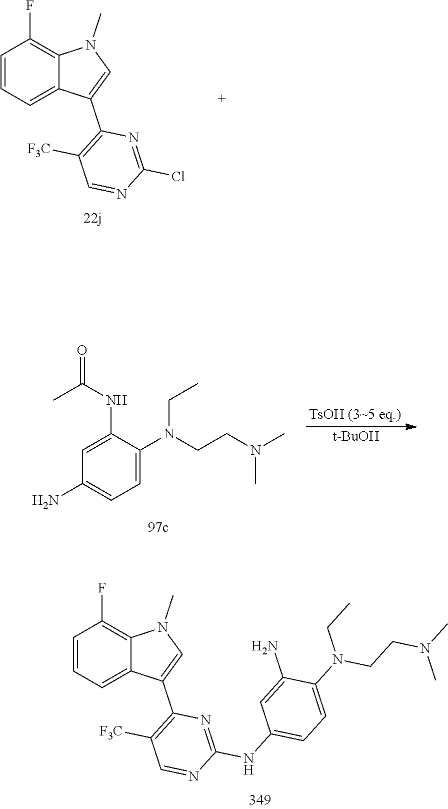

### N¹-(2-(dimethylamino)ethyl)-N¹-ethyl-N⁴-(4-(7-fluoro-1-methyl-1H-indol-3-yl)-5-(trifluoromethyl)pyrimidin-2-yl)benzene-1,2,4-triamine (349)

Synthesis of 349 was similar to that of 297. The residue was purified by preparative thin layer chromatography (dichloromethane/methanol=12/1) to give the title product 349 (50 mg, yield 21.3%) as a gray solid. ¹H NMR (500 MHz, DMSO-d₆) δ 9.83 (s, 1H), 8.71 (s, 1H), 8.10 (s, 1H), 7.85 (s, 1H), 7.12-7.03 (m, 2H), 7.01 (s, 1H), 6.95 (d, J=8.5 Hz, 1H), 6.92 (d, J=8.5 Hz, 1H), 4.08 (s, 3H), 3.11 (t, J=5.8 Hz, 2H), 2.90 (q, J=6.7 Hz, 4H), 2.59 (s, 6H), 0.90 (t, J=7.0 Hz, 3H).

### Procedure for the Synthesis of 350

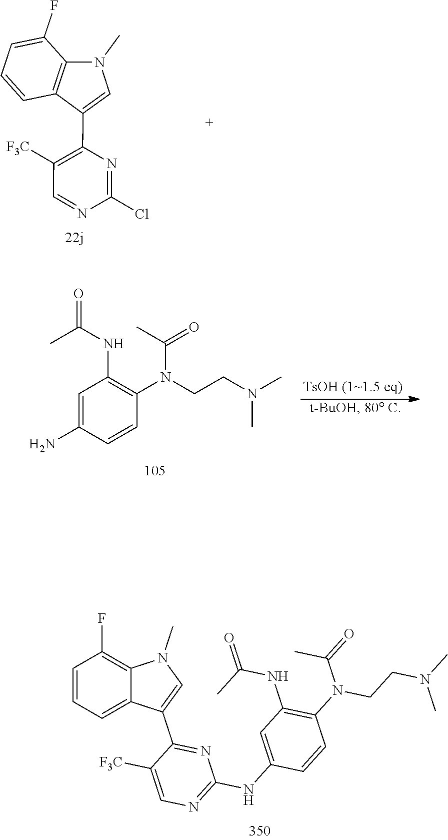

### N-(2-acetamido-4-((4-(7-fluoro-1-methyl-1H-indol-3-yl)-5-(trifluoromethyl)pyrimidin-2-yl)amino)phenyl)-N-(2-(dimethylamino)ethyl)acetamide (350)

To the mixture of 105 (175 mg, 0.63 mmol) and 22j (207 mg, 0.63 mmol) in t-BuOH (10 mL) was added TsOH (114 mg, 0.60 mmol). The mixture was stirred at 80° C. for 16 h. The mixture was concentrated and the residue was purified by preparative thin layer chromatography. The product was basified by aqueous NaHCO₃ to give 350 (70 mg, 0.12 mmol, 19.05% yield) as off-white solid. MS-ESI (M+H⁺): 572.4; ¹H NMR (500 MHz, DMSO-d₆) δ 10.79 (s, 1H), 10.27 (s, 1H), 8.80 (s, 1H), 8.30 (s, 1H), 8.08 (s, 1H), 7.87 (s, 1H), 7.72 (d, J=7.9 Hz, 1H), 7.22 (d, J=8.2 Hz, 1H), 7.16-6.92 (m, 2H), 4.49 (s, 1H), 4.08 (s, 3H), 3.09 (s, 1H), 2.44-2.33 (m, 1H), 2.32 (m, 1H), 2.27 (s, 6H), 2.00 (s, 3H), 1.59 (s, 3H).

### Procedure for the Synthesis of 351

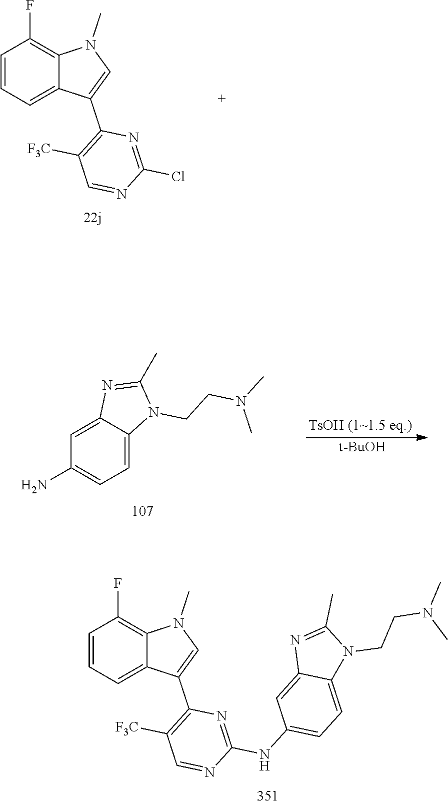

### 1-(2-(dimethylamino)ethyl)-N-(4-(7-fluoro-1-methyl-1H-indol-3-yl)-5-(trifluoromethyl)pyrimidin-2-yl)-2-methyl-1H-benzo[d]imidazol-5-amine (351)

Synthesis of 351 was similar to that of 296. The residue was purified by preparative thin layer chromatography (dichloromethane/methanol=12/1) to give the title product 351 (22 mg, yield 7.1%) as a yellow solid. MS-ESI (M+H)⁺: 512.5. ¹H NMR (500 MHz, DMSO-d₆) δ 10.03 (s, 1H), 8.74 (s, 1H), 7.92 (s, 1H), 7.85 (s, 1H), 7.53-7.47 (m, 1H), 7.40 (d, J=8.6 Hz, 1H), 7.14-6.93 (m, 2H), 4.23 (t, J=6.5 Hz, 2H), 4.08 (s, 3H), 2.55 (t, J=6.5 Hz, 2H), 2.52 (s, 3H), 2.20 (s, 6H).

### Procedure for the Synthesis of 352

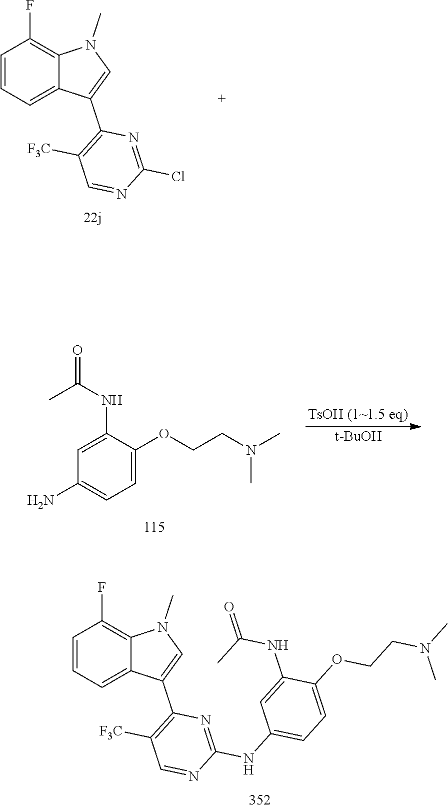

### N-(2-(2-(dimethylamino)ethoxy)-5-((4-(7-fluoro-1-methy-1H-indol-3-yl)-5-(trifluoromethyl)pyrimidin-2-yl)amino)phenyl)acetamide (352)

Synthesis of 352 was similar to that of 296. The residue was purified by preparative thin layer chromatography (dichloromethane/methanol=12/1) to give the title product 352 (41 mg, yield 12.7%) as a light yellow solid. MS-ESI (M+H)⁺: 531.3. ¹H NMR (500 MHz, DMSO-d₆) δ 10.01 (s, 1H), 9.64 (s, 1H), 8.72 (s, 1H), 8.29 (s, 1H), 7.86 (s, 1H), 7.47 (d, J=7.0 Hz, 1H), 7.04 (s, 3H), 4.11 (t, J=5.4 Hz, 2H), 4.07 (s, 3H), 2.70 (s, 2H), 2.34 (s, 6H), 2.08 (s, 3H).

### Procedure for the Synthesis of R-353

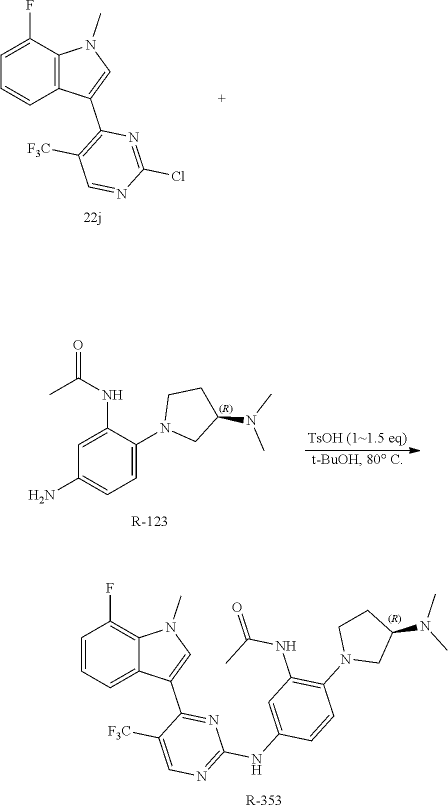

### (R)—N-(2-(3-(dimethylamino)pyrrolidin-1-yl)-5-((4-(7-fluoro-1-methyl-1H-indol-3-yl)-5-(trifluoromethyl)pyrimidin-2-yl)amino)phenyl)acetamide (R-353)

To the mixture of 22j (210 mg, 0.64 mmol) and R-123 (167 mg, 0.64 mmol) in t-BuOH (10 mL) was added TsOH (109 mg, 0.57 mmol). The mixture was stirred at 80° C. for 16 h. The mixture was concentrated and the residue was purified by preparative thin layer chromatography. The product was basified by aqueous K₂CO₃ to give R-353 (83 mg, 0.15 mmol, 23.3% yield) as off-white solid. MS-ESI (M+H⁺): 556.4; ¹H NMR (500 MHz, DMSO-d₆) δ 9.99 (s, 1H), 9.24 (s, 1H), 8.71 (s, 1H), 8.08 (s, 1H), 7.92 (s, 1H), 7.85 (s, 1H), 7.50 (d, J=7.4 Hz, 1H), 7.21-7.00 (m, 2H), 6.98-6.80 (m, 1H), 4.08 (s, 3H), 3.32-2.97 (m, 5H), 2.56 (s, 6H), 2.28-2.14 (m, 1H), 2.09 (s, 3H), 2.02 (d, J=17.8 Hz, 1H).

### Procedure for the Synthesis of R-354

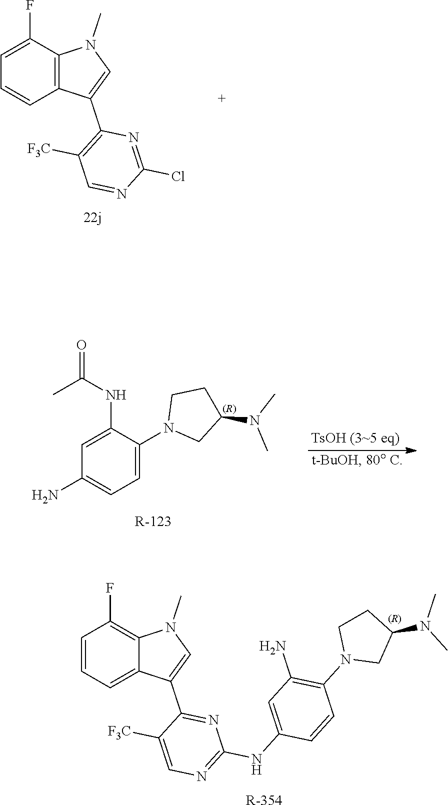

### (R)-4-(3-(dimethylamino)pyrrolidin-1-yl)-N¹-(4-(7-fluoro-1-methyl-1H-indol-3-yl)-5-(trifluoromethyl)pyrimidin-2-yl)benzene-1,3-diamine (R-354)

To the mixture of 22j (185 mg, 0.56 mmol) and R-123 (122 mg, 0.46 mmol) in t-BuOH (10 mL) was added TsOH (360 mg, 1.89 mmol). The mixture was stirred at 80° C. for 24 h. The mixture was concentrated and the residue was purified by preparative thin layer chromatography. The product was basified by aqueous NaHCO₃ to give R-354 (24.9 mg, 0.048 mmol, 10.5% yield) as off-white solid. MS-ESI (M+H⁺): 514.6; ¹H NMR (500 MHz, Chloroform-d) δ 8.63 (s, 1H), 8.08 (d, J=8.1 Hz, 1H), 7.58 (s, 1H), 7.37 (s, 1H), 7.29 (s, 1H), 7.11-7.05 (m, 1H), 6.95 (dd, J=13.7, 8.1 Hz, 2H), 6.80 (d, J=7.1 Hz, 1H), 4.07 (s, 3H), 3.90 (s, 2H), 3.22-3.12 (m, 2H), 3.08-3.00 (m, 2H), 2.88 (m, 1H), 2.28 (s, 6H), 2.12 (m, 1H), 1.87 (m, 1H).

### Procedure for the Synthesis of S-353

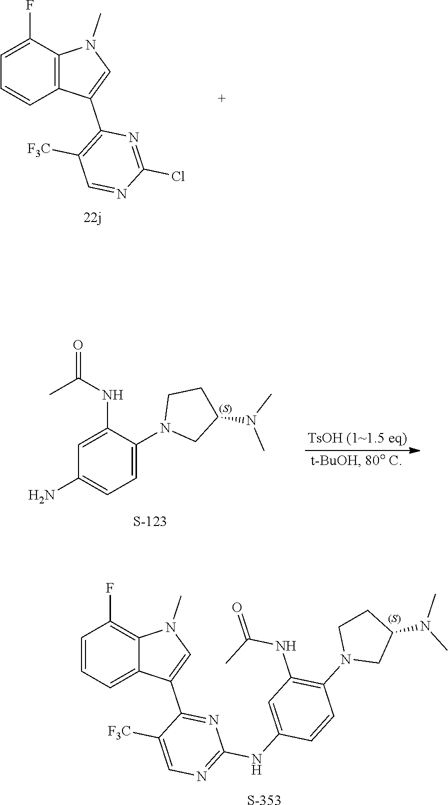

### (S)—N-(2-(3-(dimethylamino)pyrrolidin-1-yl)-5-((4-(7-fluoro-1-methyl-1H-indol-3-yl)-5-(trifluoromethyl)pyrimidin-2-yl)amino)phenyl)acetamide (S-353)

To the mixture of 22j (202 mg, 0.62 mmol) and S-123 (169 mg, 0.62 mmol) in t-BuOH (10 mL) was added TsOH (116 mg, 0.61 mmol). The mixture was stirred at 80° C. for 16 h. The mixture was concentrated and the residue was purified by preparative thin layer chromatography. The product was basified by aqueous NaHCO₃ to give S-353 (48 mg, 0.086 mmol, 14% yield) as off-white solid. MS-ESI (M+H⁺): 556.8; ¹H NMR (500 MHz, DMSO-d₆) δ 9.93 (s, 1H), 9.17 (s, 1H), 8.70 (s, 1H), 8.08 (s, 1H), 7.84 (s, 1H), 7.73 (s, 1H), 7.47 (d, J=6.6 Hz, 1H), 7.13-6.98 (m, 2H), 6.80 (s, 1H), 4.07 (s, 3H), 3.29 (q, J=8.7 Hz, 1H), 3.14 (d, J=7.4 Hz, 2H), 3.06 (t, J=7.4 Hz, 1H), 2.75-2.62 (m, 1H), 2.16 (s, 6H), 2.09-2.03 (m, 1H), 2.00 (s, 3H), 1.76-1.66 (m, 1H).

### Procedure for the Synthesis of S-354

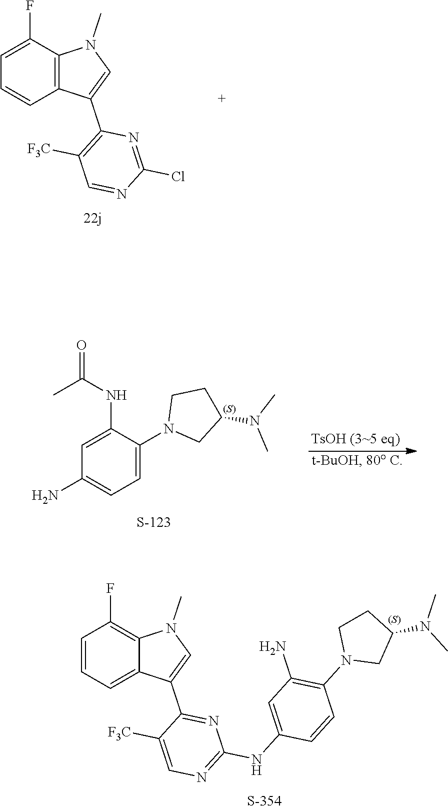

### (S)-4-(3-(dimethylamino)pyrrolidin-1-yl)-N¹-(4-(7-fluoro-1-methyl-1H-indol-3-yl)-5-(trifluoromethyl)pyrimidin-2-yl)benzene-1,3-diamine (S-354)

To the mixture of 22j (185 mg, 0.56 mmol) and S-123 (122 mg, 0.46 mmol) in t-BuOH (10 mL) was added TsOH (360 mg, 1.89 mmol). The mixture was stirred at 80° C. for 24 h. The mixture was concentrated and the residue was purified by preparative thin layer chromatography. The product was basified by aqueous NaHCO₃ to give S-354 (27.9 mg, 0.054 mmol, 11.7% yield) as off-white solid. MS-ESI (M+H⁺): 514.7; ¹H NMR (500 MHz, Chloroform-d) δ 8.63 (s, 1H), 8.08 (d, J=8.0 Hz, 1H), 7.58 (s, 1H), 7.39 (s, 1H), 7.29 (s, 1H), 7.08 (m, 1H), 6.95 (dd, J=13.1, 8.1 Hz, 2H), 6.80 (d, J=7.5 Hz, 1H), 4.07 (s, 3H), 3.90 (s, 2H), 3.17 (m, 2H), 3.05 (m, 2H), 2.88 (m, 1H), 2.29 (s, 6H), 2.13 (m, 1H), 1.87 (m, 1H).

### Procedure for the Synthesis of 355

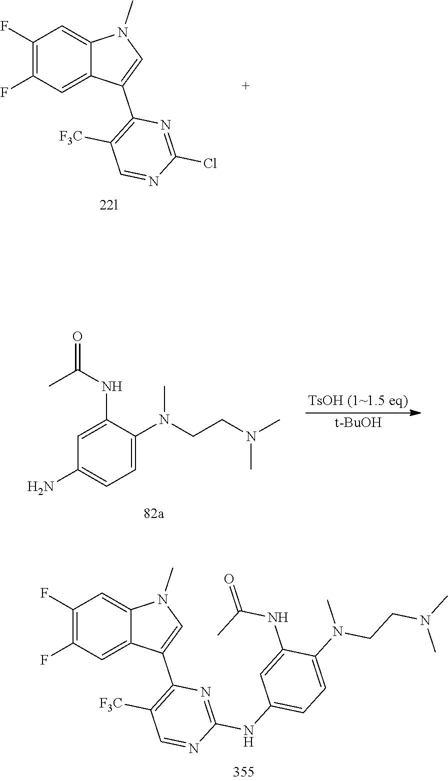

### N-(5-((4-(5,6-difluoro-1-methyl-1H-indol-3-yl)-5-(trifluoromethyl) pyrimidin-2-yl)amino)-2-((2-(dimethylamino)ethyl)(methyl)amino)phenyl)acetamide (355)

Synthesis of 355 was similar to that of 296. The residue was purified by preparative thin layer chromatography (dichloromethane/methanol=12/1) to give the title product 355 (57 mg, yield 23.5%) as a gray solid. MS-ESI (M+H)⁺: 562.3. ¹H NMR (500 MHz, DMSO-d₆) δ 10.86 (s, 1H), 10.07 (s, 1H), 9.68 (s, 1H), 8.70 (s, 1H), 8.34 (s, 1H), 7.99 (s, 1H), 7.71 (dd, J=11.1, 7.0 Hz, 1H), 7.50 (s, 1H), 7.20 (d, J=8.7 Hz, 1H), 3.90 (s, 3H), 3.20-3.10 (m, 4H), 2.65 (s, 6H), 2.57 (s, 3H), 2.23 (s, 3H).

### Procedure for the Synthesis of 356

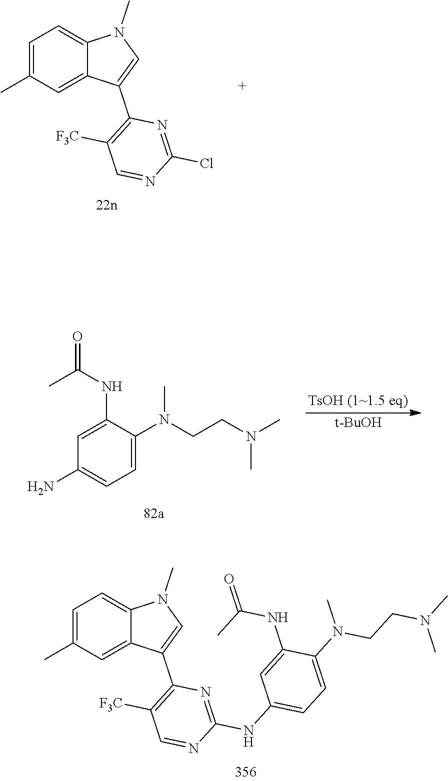

### N-(5-((4-(1,5-dimethyl-1H-indol-3-yl)-5-(trifluoromethyl)pyrimidin-2-yl)amino)-2-((2-(dimethylamino)ethyl)(methyl)amino)phenyl)acetamide (356)

Synthesis of 356 was similar to that of 296. The residue was purified by flash column chromatography (dichloromethane/methanol=15/1) and by preparative thin layer chromatography (dichloromethane/methanol=12/1) to give the title product 356 (40 mg, yield 15.1%) as a gray solid. LC-MS: m/z 540.4 [M+H]⁺. ¹H NMR (500 MHz, DMSO-d₆) δ 9.99 (s, 2H), 8.68 (s, 1H), 8.38 (s, 1H), 8.13 (s, 1H), 7.84 (s, 1H), 7.64 (d, J=8.5 Hz, 1H), 7.41 (d, J=8.3 Hz, 1H), 7.20 (d, J=8.7 Hz, 1H), 7.10 (dd, J=8.3, 1.7 Hz, 1H), 3.88 (s, 3H), 2.81 (t, J=5.3 Hz, 2H), 2.65 (s, 3H), 2.36 (s, 2H), 2.24 (s, 6H), 2.06 (s, 3H).

### Procedure for the Synthesis of 357

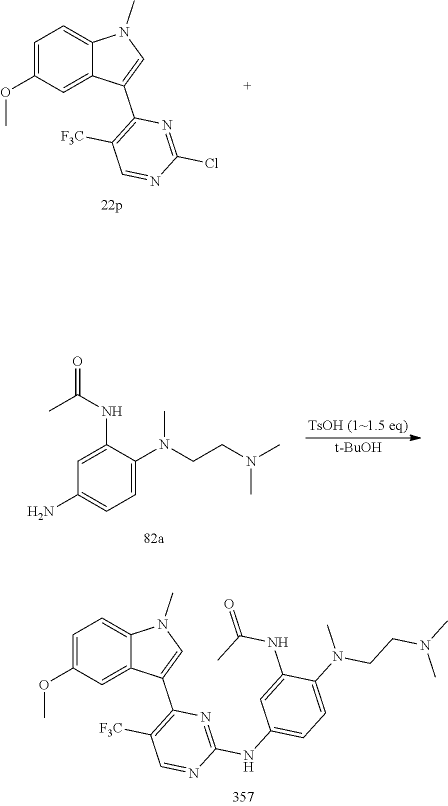

### N-(2-((2-(dimethylamino)ethyl)(methyl)amino)-5-((4-(5-methoxy-1-methyl-1H-indol-3-yl) 5-(trifluoromethyl)pyrimidin-2-yl)amino)phenyl)acetamide (357)

Synthesis of 357 was similar to that of 296. The residue was purified by preparative thin layer chromatography (dichloromethane/methanol=12/1) to give the title product 357 (56 mg, yield 25.3%) as a gray solid. MS-ESI (M+H)⁺: 556.6. ¹H NMR (500 MHz, DMSO-d₆) δ 10.05 (s, 1H), 9.89 (s, 1H), 8.70 (s, 1H), 8.36 (s, 1H), 7.84 (s, 1H), 7.81 (s, 1H), 7.65 (d, J=9.2 Hz, 1H), 7.43 (d, J=8.9 Hz, 1H), 7.16 (d, J=8.7 Hz, 1H), 6.90 (dd, J=8.8, 2.5 Hz, 1H), 3.88 (s, 3H), 3.64 (s, 3H), 2.89 (t, J=5.6 Hz, 2H), 2.55 (s, 1H), 2.36 (s, 6H), 2.10 (s, 3H).

### Procedure for the Synthesis of 358

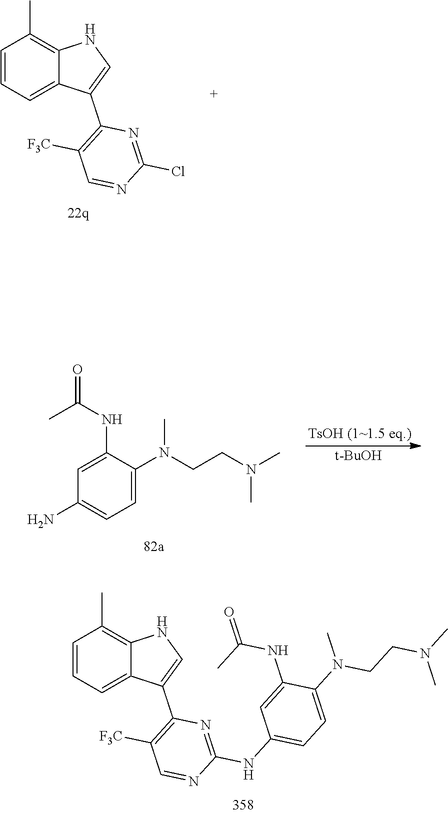

### N-(2-((2-(dimethylamino)ethyl)(methyl)amino)-5-((4-(7-methyl-1H-indol-3-yl)-5-(trifluoromethyl)pyrimidin-2-yl)amino)phenyl)acetamide (358)

Synthesis of 358 was similar to that of 296. The residue was purified by preparative thin layer chromatography (dichloromethane/methanol=12/1) to give the title product 358 (15 mg, yield 4.4%) as a white solid. MS-ESI (M+H)⁺: 526.7. ¹H NMR (500 MHz, DMSO-d₆) δ 11.84 (s, 1H), 10.03 (s, 1H), 9.98 (s, 1H), 8.70 (s, 1H), 8.43 (s, 1H), 8.14 (s, 1H), 7.82 (s, 1H), 7.54 (d, J=6.5 Hz, 1H), 7.21 (d, J=8.5 Hz, 1H), 7.01 (s, 2H), 2.84-2.76 (m, 2H), 2.66 (s, 3H), 2.32-2.26 (m, 2H), 2.23 (s, 6H), 2.07 (s, 3H).

### Procedure for the Synthesis of 359

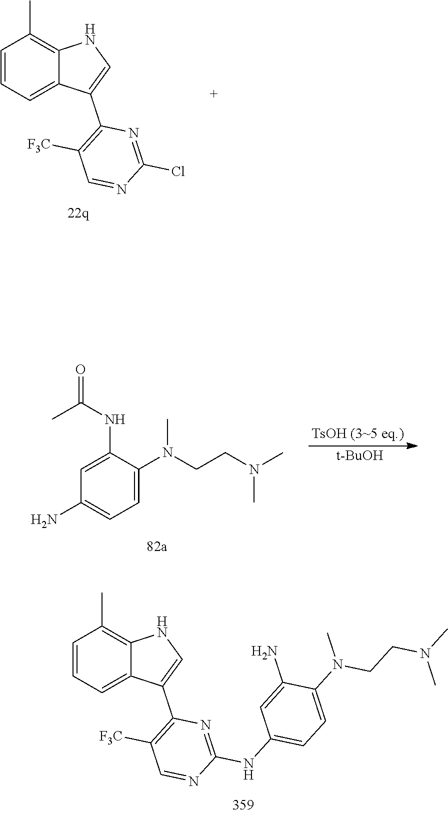

### N¹-(2-(dimethylamino)ethyl)-N¹-methyl-N⁴-(4-(7-methyl-1H-indol-3-yl)-5-(trifluoromethyl)pyrimidin-2-yl)benzene-1,2,4-triamine (359)

Synthesis of 359 was similar to that of 297. The residue was purified by preparative thin layer chromatography (dichloromethane/methanol=12/1) to give the title product 359 (18 mg, yield 7.8%) as a dark brown solid. MS-ESI (M+H)⁺: 484.3. ¹H NMR (500 MHz, DMSO-d₆) δ 11.91 (s, 1H), 9.76 (s, 1H), 8.68 (s, 1H), 8.15 (s, 1H), 7.80 (s, 1H), 7.03 (q, J=7.1 Hz, 3H), 6.97 (dd, J=8.4, 1.9 Hz, 1H), 6.90 (d, J=8.5 Hz, 1H), 3.17 (s, 2H), 3.10 (s, 2H), 2.69 (s, 6H), 2.52 (s, 3H), 2.50 (s, 3H).

### Procedure for the Synthesis of 362

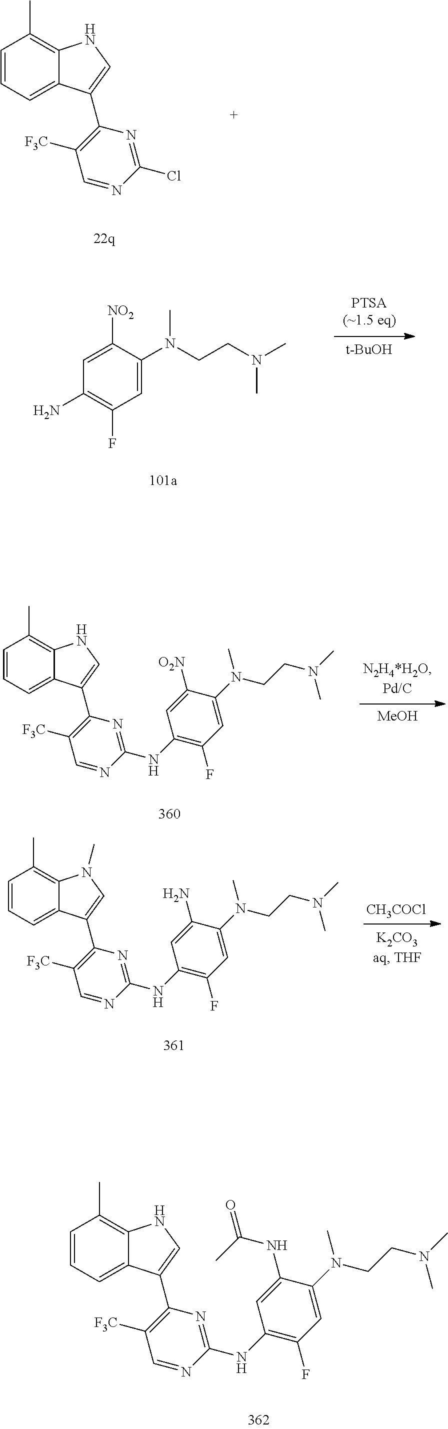

### N¹-(2-(dimethylamino)ethyl)-5-fluoro-N¹-methyl-N⁴-(4-(7-methyl-1H-indol-3-yl)-5-(trifluoromethyl)pyrimidin-2-yl)-2-nitrobenzene-1,4-diamine (360)

Synthesis of 360 was similar to that of 296 by using 22q (223 mg, 0.72 mmol) and 101a (162 mg, 0.63 mmol) to afford the title compound 360 (130 mg, yield 38.7%).

### N¹-(2-(dimethylamino)ethyl)-5-fluoro-N¹-methyl-N⁴-(4-(7-methyl-1H-indol-3-yl)-5-(trifluoromethyl)pyrimidin-2-yl)benzene-1,2,4-triamine (361)

A mixture of above compound 360 (130 mg, 0.24 mmol), Pd/C (5% on activated carbon, 72 mg, 0.03 mmol) and hydrazine hydrate (80%, 1 ml, 16 mmol) in methanol (10 ml) was heated at refluxing temperature for 2 hrs. After cooling to room temperature, the reaction mixtures was filtered through Celite® and the filtrates were concentrated to remove methanol. The residual was extracted with ethyl acetate. The organic layer was washed with brine, dried over Na₂SO₄, filtered and concentrated to afford the crude title compound 361 (125 mg), which was directly used in the next step without further purification. LC-MS: m/z 502.5 [M+H]⁺.

### N-(2-((2-(dimethylamino)ethyl)(methyl)amino)-4-fluoro-5-((4-(7-methyl-1H-indol-3-yl)-5-(trifluoromethyl)pyrimidin-2-yl)amino)phenyl)acetamide (362)

Acetyl chloride (40 mg, 0.51 mmol) was dropped to the mixture of 361 (125 mg, 0.25 mmol) and saturated K₂CO₃ aqueous (0.5 ml) in tetrahydrofuran (5 mL) at 0° C. The mixture was stirred for 1 hr. Then the mixture was quenched with H₂O and extracted with ethyl acetate. The organic layer was separated, dried over Na₂SO₄, and then concentrated under reduced pressure. The residue was purified by preparative thin layer chromatography (dichloromethane/methanol=8/1) to give the title compound 362 (37 mg, yield 27.8% over two steps) as an off-white solid. LC-MS: m/z 544.5 [M+H]⁺.

### Procedure for the Synthesis of 363

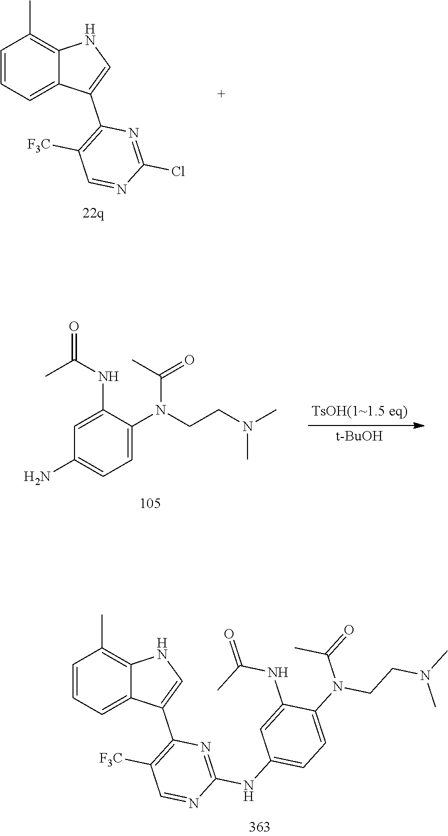

### N-(2-acetamido-4-((4-(7-methyl-1H-indol-3-yl)-5-(trifluoro methyl)pyrimidin-2-yl)amino)phenyl)-N-(2-(dimethylamino)ethyl)acetamide (363)

A mixture of 22q (0.15 g, 0.48 mmol), 105 (0.13 g, 0.47 mmol), and TsOH (0.08 g, 0.47 mmol) in t-BuOH (20 mL) was stirred at 100° C. overnight. After cooling to room temperature, the mixture was neutralized with saturated NaHCO₃ and concentrated under reduced pressure. The residue was dissolved in dichloromethane (40 mL), and washed with H₂O (15 mL*2), dried with Na₂SO₄. After concentration, the residue was purified by preparative thin layer chromatography (dichloromethane/methanol=12/1) to give the desired product 363 (27 mg, yield 11.3%) as a yellow solid. MS-ESI (M+Na)⁺: 576.3. ¹H NMR (500 MHz, DMSO-d₆) δ 11.87 (s, 1H), 10.77 (s, 1H), 10.20 (s, 1H), 8.77 (s, 1H), 8.30 (s, 1H), 8.15 (s, 1H), 7.83 (d, J=2.1 Hz, 1H), 7.77 (dd, J=8.6, 2.3 Hz, 1H), 7.22 (d, J=8.6 Hz, 1H), 7.06-6.99 (m, 2H), 4.50 (t, J=9.6 Hz, 1H), 3.10 (d, J=14.4 Hz, 1H), 2.53 (s, 3H), 2.38 (dd, J=12.9, 4.3 Hz, 1H), 2.34-2.27 (m, 1H), 2.24 (s, 6H), 2.01 (s, 3H), 1.61 (s, 3H).

### Procedure for the Synthesis of 364

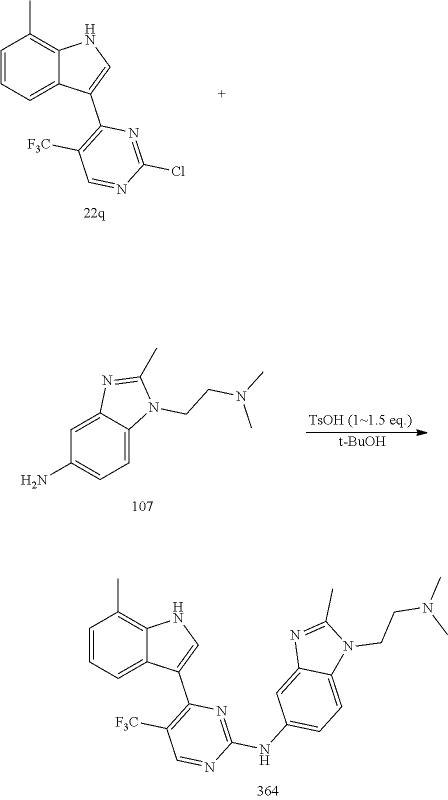

### 1-(2-(dimethylamino)ethyl)-2-methyl-N-(4-(7-methyl-1H-indol-3-yl)-5-(trifluoromethyl)pyrimidin-2-yl)-1H-benzo[d]imidazol-5-amine (364)

Synthesis of 364 was similar to that of 926. The residue was purified by preparative thin layer chromatography (dichloromethane/methanol=12/1) to give the title product 364 (29 mg, yield 9.2%) as a yellow solid. MS-ESI (M+H)⁺: 494.4. ¹H NMR (500 MHz, DMSO-d₆) δ 11.94 (s, 1H), 9.98 (s, 1H), 8.72 (s, 1H), 7.95 (s, 1H), 7.81 (s, 1H), 7.53 (dd, J=8.7, 1.8 Hz, 1H), 7.41 (d, J=8.6 Hz, 1H), 7.08-6.86 (m, 2H), 4.26 (s, 2H), 2.60 (t, J=5.3 Hz, 2H), 2.53 (s, 3H), 2.52 (s, 3H), 2.23 (s, 6H).

### Procedure for the Synthesis of 365

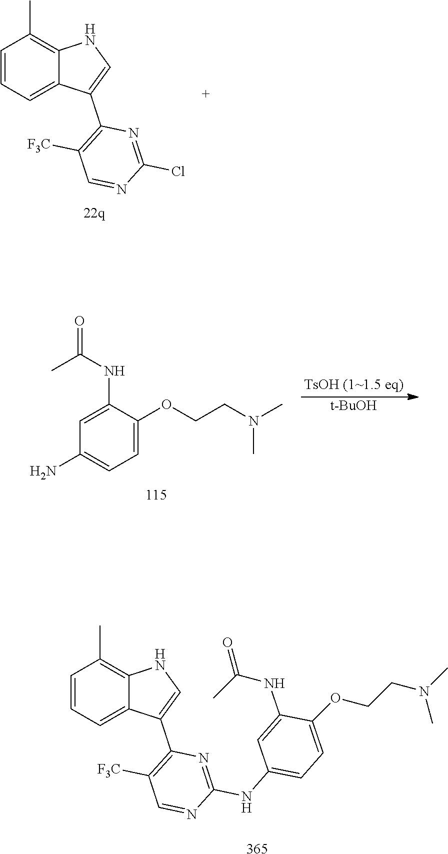

### N-(2-(2-(dimethylamino)ethoxy)-5-((4-(7-methy-1H-indol-3-yl)-5-(trifluoromethyl)pyrimidin-2-yl)amino)phenyl)acetamide (365)

Synthesis of 365 was similar to that of 296. The residue was purified by preparative thin layer chromatography (dichloromethane/methanol=12/1) to give the title product 365 (112 mg, yield 34.1%) as a gray solid. MS-ESI (M+H)⁺: 513.6. ¹H NMR (500 MHz, DMSO-d₆) δ 11.84 (s, 1H), 9.95 (s, 1H), 9.62 (s, 1H), 8.70 (s, 1H), 8.28 (s, 1H), 8.10 (s, 1H), 7.81 (s, 1H), 7.59-7.45 (m, 1H), 7.12-6.94 (m, 3H), 4.09 (t, J=5.6 Hz, 2H), 2.57 (t, J=5.4 Hz, 2H), 2.51 (s, 3H), 2.27 (s, 6H), 2.06 (s, 3H).

### Procedure for the Synthesis of R-366

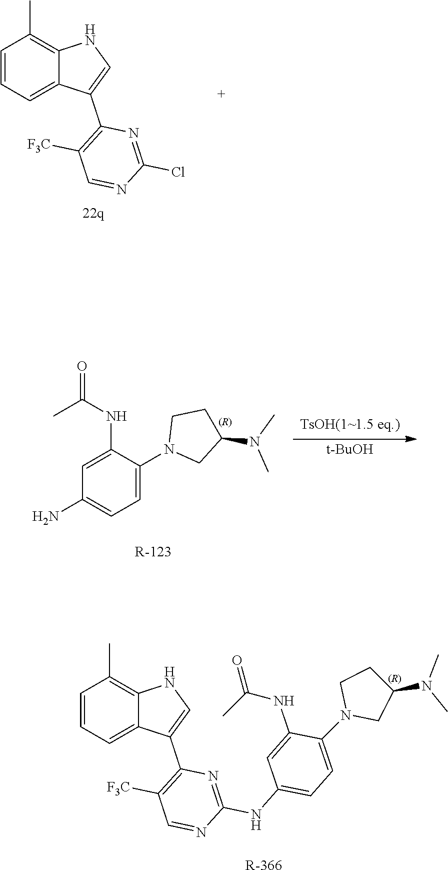

### (R)—N-(2-(3-(dimethylamino)pyrrolidin-1-yl)-5-((4-(7-methyl-1H-indol-3-yl)-5-(trifluoromethyl)pyrimidin-2-yl)amino)phenyl)acetamide (R-366)

Synthesis of R-366 was similar to that of 296. The residue was purified by preparative thin layer chromatography (dichloromethane/methanol=12/1) to give the title product R-366 (36 mg, yield 11.0%) as a light yellow solid. MS-ESI (M+H)⁺: 538.3. ¹H NMR (500 MHz, DMSO-d₆) δ 11.90 (s, 1H), 9.91 (s, 1H), 9.22 (s, 1H), 8.69 (s, 1H), 8.12 (s, 1H), 7.84 (s, 1H), 7.81 (s, 1H), 7.53 (d, J=8.4 Hz, 1H), 7.03 (t, J=9.4 Hz, 2H), 6.87 (d, J=5.1 Hz, 1H), 3.26 (s, 2H), 3.16 (s, 4H), 2.52 (s, 3H), 2.40 (s, 6H), 2.15 (s, 1H), 2.06 (s, 3H), 1.91 (s, 1H).

### Procedure for the Synthesis of S-366

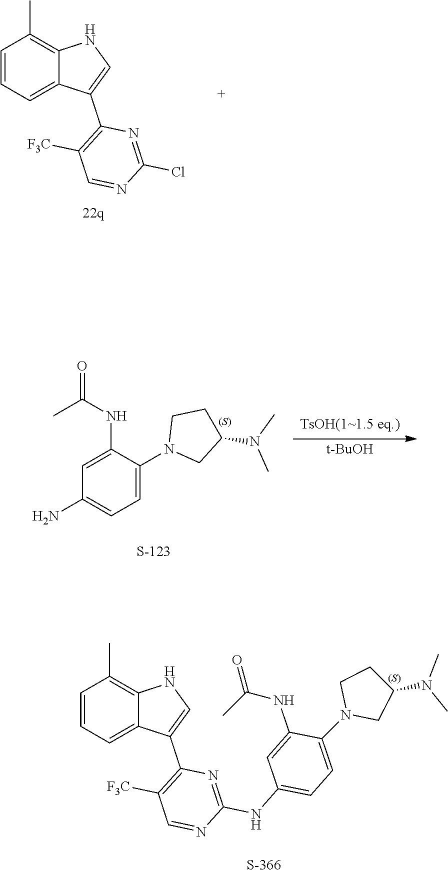

### (S)—N-(2-(3-(dimethylamino)pyrrolidin-1-yl)-5-((4-(7-methyl-1H-indol-3-yl)-5-(trifluoromethyl)pyrimidin-2-yl)amino)phenyl)acetamide (S-366)

Synthesis of S-366 was similar to that of R-366. The residue was purified by preparative thin layer chromatography (dichloromethane/methanol=12/1) to give the title product S-366 (113 mg, yield 32.8%) as a yellow solid. MS-ESI (M+H)⁺: 538.5. ¹H NMR (500 MHz, DMSO-d₆) δ 11.92 (s, 1H), 9.92 (s, 1H), 9.24 (s, 1H), 8.70 (s, 1H), 8.12 (s, 1H), 7.89 (s, 1H), 7.81 (s, 1H), 7.54 (d, J=8.6 Hz, 1H), 7.03 (t, J=9.7 Hz, 2H), 6.93-6.86 (m, 1H), 3.31 (s, 1H), 3.17 (dd, J=18.8, 11.3 Hz, 3H), 3.12-3.04 (m, 1H), 2.52 (s, 3H), 2.47 (s, 5H), 2.18 (s, 1H), 2.08 (s, 3H), 1.97 (s, 1H).

### Procedure for the Synthesis of R-367

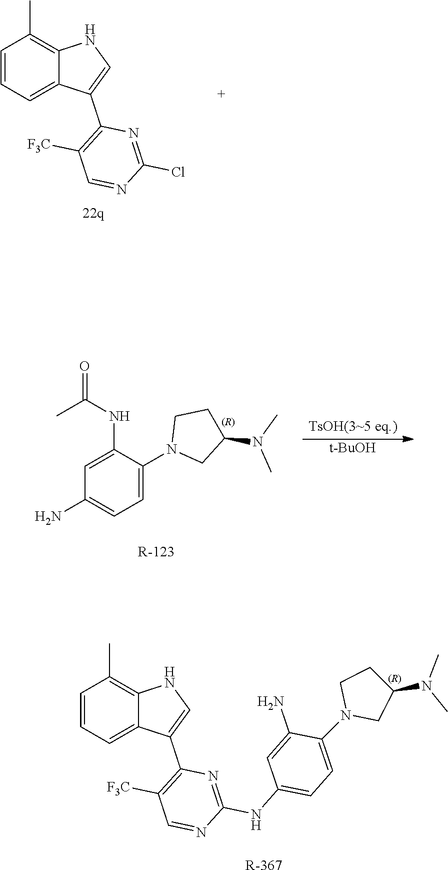

### (R)-4-(3-(dimethylamino)pyrrolidin-1-yl)-N¹-(4-(7-methyl-1H-indol-3-yl)-5-(trifluoromethyl)pyrimidin-2-yl)benzene-1,3-diamine (R-367)

A mixture of 22q (0.20 g, 0.64 mmol), R-123 (0.17 g, 0.65 mmol), and TsOH (0.44 g, 2.56 mmol) in t-BuOH (15 mL) was stirred at 100° C. overnight. After cooling to room temperature, the mixture was neutralized with saturated NaHCO₃ and concentrated under reduced pressure. The residue was dissolved in dichloromethane (40 mL), and washed with H₂O (10 mL*2), dried with Na₂SO₄. After concentration, the residue was purified by preparative thin layer chromatography (dichloromethane/methanol=11/1) to give the desired product R-367 (44 mg, yield 13.84%) as a light yellow solid. MS-ESI (M+H)⁺: 496.6. ¹H NMR (500 MHz, DMSO-d₆) δ 11.85 (s, 1H), 9.70 (s, 1H), 8.67 (s, 1H), 8.13 (s, 1H), 7.80 (s, 1H), 7.07-7.03 (m, 2H), 7.01 (d, J=6.8 Hz, 1H), 6.96 (dd, J=8.5, 2.2 Hz, 1H), 6.83 (d, J=8.5 Hz, 1H), 4.68 (s, 2H), 3.04 (ddt, J=35.5, 14.2, 7.0 Hz, 5H), 2.52 (s, 3H), 2.42 (s, 6H), 2.12 (dq, J=12.8, 7.2 Hz, 1H), 1.89 (dq, J=13.6, 7.1 Hz, 1H).

### Procedure for the Synthesis of S-367

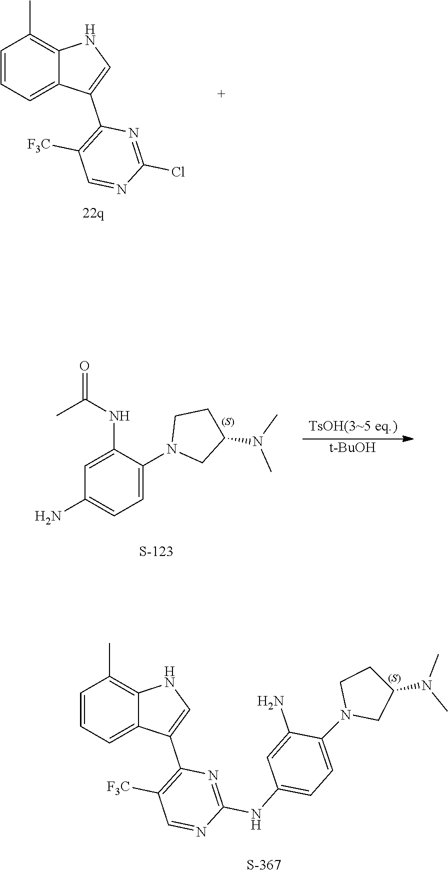

### (S)-4-(3-(dimethylamino)pyrrolidin-1-yl)-N¹-(4-(7-methyl-1H-indol-3-yl)-5-(trifluoromethyl)pyrimidin-2-yl)benzene-1,3-diamine (S-367)

Synthesis of S-367 was similar to that of R-367. The residue was purified by preparative thin layer chromatography (dichloromethane/methanol=12/1) to give the title product S-367 (36 mg, yield 11.3%) as a light yellow solid. ¹H NMR (500 MHz, DMSO-d₆) δ 11.86 (s, 1H), 9.71 (s, 1H), 8.67 (s, 1H), 8.14 (s, 1H), 7.80 (d, J=2.1 Hz, 1H), 7.06 (s, 1H), 7.05-6.99 (m, 2H), 6.96 (dd, J=8.5, 2.2 Hz, 1H), 6.83 (d, J=8.5 Hz, 1H), 4.69 (s, 2H), 3.25 (s, 1H), 3.07 (q, J=8.1, 6.3 Hz, 2H), 3.03-2.96 (m, 2H), 2.52 (s, 3H), 2.42 (s, 6H), 2.12 (dq, J=12.6, 7.3 Hz, 1H), 1.90 (dq, J=13.5, 7.2 Hz, 1H).

### Procedure for the Synthesis of 368

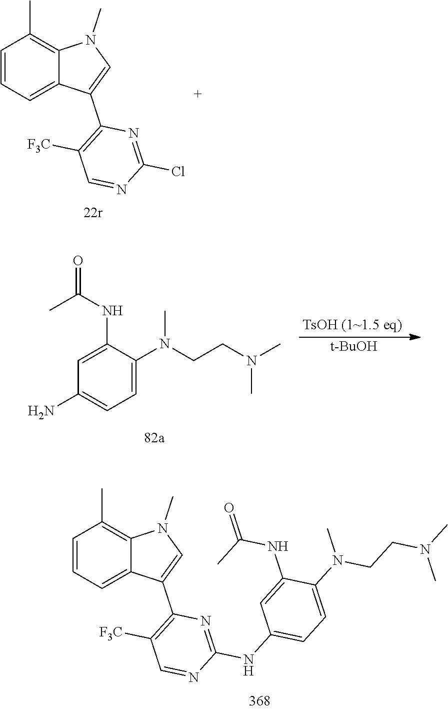

### N-(5-((4-(1,7-dimethyl-1H-indol-3-yl)-5-(trifluoromethyl)pyrimidin-2-yl)amino)-2-((2-(dimethylamino)ethyl)(methyl)amino)phenyl)acetamide (368)

Synthesis of 368 was similar to that of 296. The residue was purified by preparative thin layer chromatography (dichloromethane/methanol=12/1) to give the title product 368 (35 mg, yield 10.8%) as a light purple solid. MS-ESI (M+H)⁺: 540.4. ¹H NMR (500 MHz, DMSO-d₆) δ 10.01 (s, 1H), 9.85 (s, 1H), 8.70 (s, 1H), 8.38 (s, 1H), 8.12 (s, 1H), 7.75 (s, 1H), 7.59-7.51 (m, 1H), 7.18 (d, J=8.3 Hz, 1H), 6.96 (s, 2H), 4.17 (s, 3H), 2.94 (s, 2H), 2.77 (s, 3H), 2.64 (s, 2H), 2.61 (s, 3H), 2.42 (s, 6H), 2.13 (s, 3H).

### Procedure for the Synthesis of 369

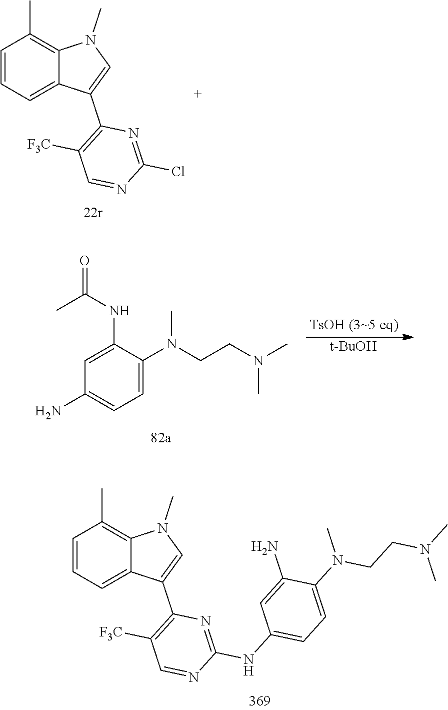

### N⁴-(4-(1,7-dimethyl-1H-indol-3-yl)-5-(trifluoromethyl)pyrimidin-2-yl)-N¹-(2-(dimethylamino)ethyl)-N¹-methylbenzene-1,2,4-triamine (369)

Synthesis of 369 was similar to that of 297. The residue was purified by preparative thin layer chromatography (dichloromethane/methanol=12/1) to give the title product 369 (42 mg, yield 15.1%) as a off-white solid. MS-ESI (M+H)⁺: 498.5. ¹H NMR (500 MHz, DMSO-d₆) δ 9.76 (s, 1H), 8.67 (s, 1H), 8.13 (s, 1H), 7.72 (s, 1H), 7.06-6.98 (m, 2H), 6.98-6.92 (m, 2H), 6.88 (d, J=8.4 Hz, 1H), 4.16 (s, 3H), 3.02 (d, J=22.0 Hz, 4H), 2.77 (s, 3H), 2.58 (s, 6H), 2.51 (s, 3H).

### Procedure for the Synthesis of 370

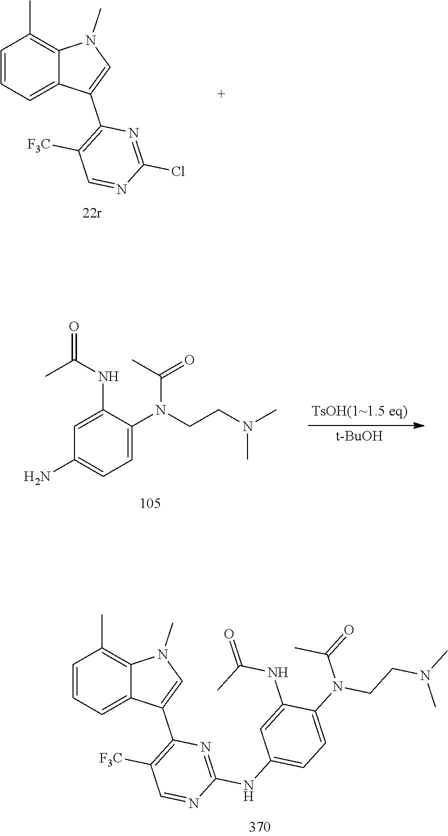

### N-(2-acetamido-4-((4-(1,7-dimethyl-1H-indol-3-yl)-5-(trifluoro methyl)pyrimidin-2-yl)amino)phenyl)-N-(2-(dimethylamino)ethyl)acetamide (370)

Synthesis of 370 was similar to that of 296. The residue was purified by preparative thin layer chromatography (dichloromethane/methanol=12/1) to give the title product 370 (88 mg, yield 25.2%) as a yellow solid. MS-ESI (M+H)⁺: 568.7. ¹H NMR (500 MHz, DMSO-d₆) δ 10.73 (s, 1H), 10.22 (s, 1H), 8.77 (s, 1H), 8.31 (s, 1H), 8.14 (s, 1H), 7.77 (d, J=2.4 Hz, 1H), 7.75 (s, 1H), 7.21 (d, J=8.6 Hz, 1H), 7.00 (t, J=7.5 Hz, 1H), 6.96 (d, J=6.9 Hz, 1H), 4.46 (s, 1H), 4.16 (s, 3H), 3.13 (d, J=14.2 Hz, 1H), 2.77 (s, 3H), 2.39 (d, J=12.6 Hz, 1H), 2.33 (t, J=10.8 Hz, 1H), 2.25 (s, 6H), 2.02 (s, 3H), 1.61 (s, 3H).

### Procedure for the Synthesis of 371

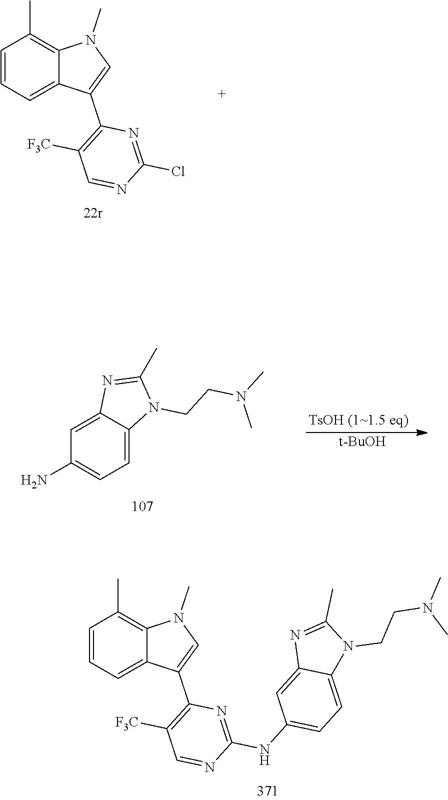

### N-(4-(1,7-dimethyl-1H-indol-3-yl)-5-(trifluoromethyl)pyrimidin-2-yl)-1-(2-(dimethylamino)ethyl)-2-methyl-1H-benzo[d]imidazol-5-amine (371)

Synthesis of 371 was similar to that of 296. The residue was purified by preparative thin layer chromatography (dichloromethane/methanol=12/1) to give the title product 371 (44 mg, yield 14.1%) as a yellow solid. MS-ESI (M+H)⁺: 508.5. ¹H NMR (500 MHz, DMSO-d₆) δ 9.98 (s, 1H), 8.72 (s, 1H), 7.95 (s, 1H), 7.73 (s, 1H), 7.52 (dd, J=8.7, 1.8 Hz, 1H), 7.40 (d, J=8.5 Hz, 1H), 7.05-6.78 (m, 2H), 4.24 (t, J=6.5 Hz, 2H), 4.16 (s, 3H), 2.77 (s, 3H), 2.59 (t, J=6.0 Hz, 2H), 2.52 (s, 3H), 2.23 (s, 6H).

### Procedure for the Synthesis of 372

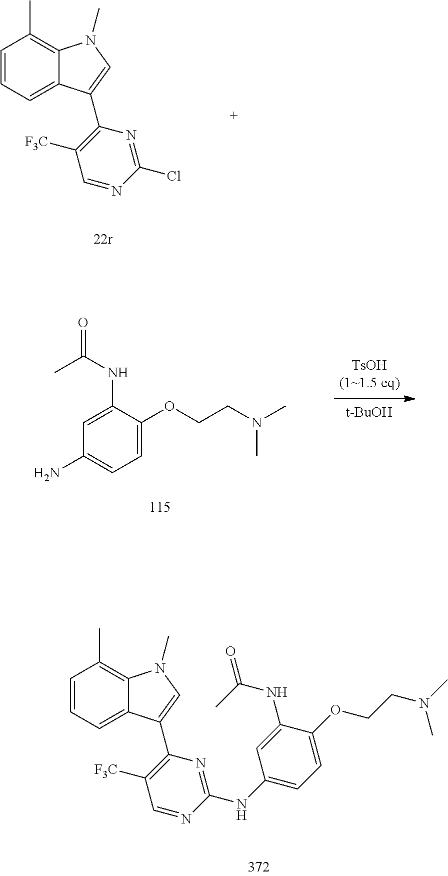

### N-(5-((4-(1,7-dimethyl-1H-indol-3-yl)-5-(trifluoromethyl)pyrimidin-2-yl)amino)-2-(2-(dimethylamino)ethoxy)phenyl)acetamide (372)

Synthesis of 372 was similar to that of 296. The residue was purified by preparative thin layer chromatography (dichloromethane/methanol=12/1) to give the title product 372 (34 mg, yield 10.5%) as a light purple solid. MS-ESI (M+H)⁺: 527.6. ¹H NMR (500 MHz, DMSO-d₆) δ 9.95 (s, 1H), 9.63 (s, 1H), 8.69 (s, 1H), 8.28 (s, 1H), 7.73 (s, 1H), 7.50 (d, J=8.5 Hz, 1H), 7.01 (d, J=8.8 Hz, 1H), 7.00-6.90 (m, 2H), 4.16 (s, 3H), 4.12 (t, J=5.3 Hz, 2H), 2.76 (s, 3H), 2.74 (s, 2H), 2.37 (s, 6H), 2.08 (s, 3H).

### Procedure for the Synthesis of R-373

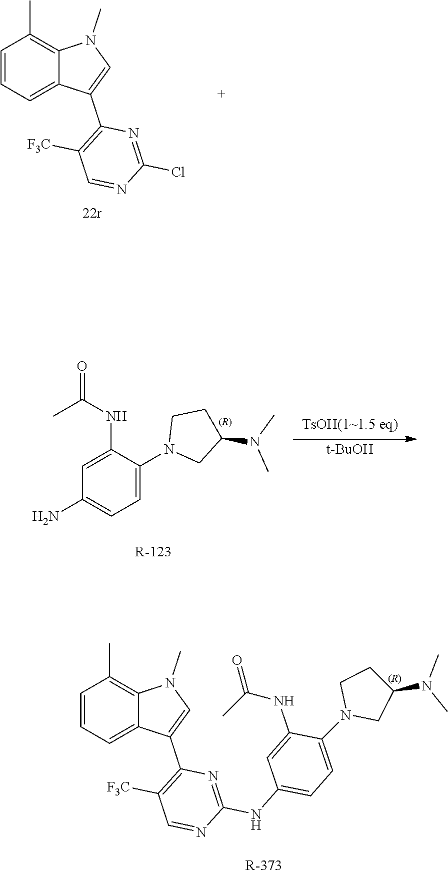

### (R)—N-(5-((4-(1,7-dimethyl-1H-indol-3-yl)-5-(trifluoromethyl) pyrimidin-2-yl)amino)-2-(3-(dimethylamino)pyrrolidin-1-yl)phenyl)acetamide (R-373)

Synthesis of R-373 was similar to that of 296. The residue was purified by preparative thin layer chromatography (dichloromethane/methanol=12/1) to give the title product R-373 (79 mg, yield 23.3%) as a off-white solid. MS-ESI (M+H)⁺: 552.6. ¹H NMR (500 MHz, DMSO-d₆) δ 9.92 (s, 1H), 9.19 (s, 1H), 8.69 (s, 1H), 8.10 (s, 1H), 7.87 (s, 1H), 7.73 (s, 1H), 7.53 (d, J=7.9 Hz, 1H), 7.00 (d, J=6.9 Hz, 1H), 6.96 (d, J=6.7 Hz, 1H), 6.88 (d, J=5.2 Hz, 1H), 4.17 (s, 3H), 3.29 (s, 2H), 3.21-3.12 (m, 3H), 2.77 (s, 3H), 2.48 (s, 5H), 2.18 (s, 1H), 2.07 (s, 3H), 1.96 (s, 1H).

### Procedure for the Synthesis of S-373

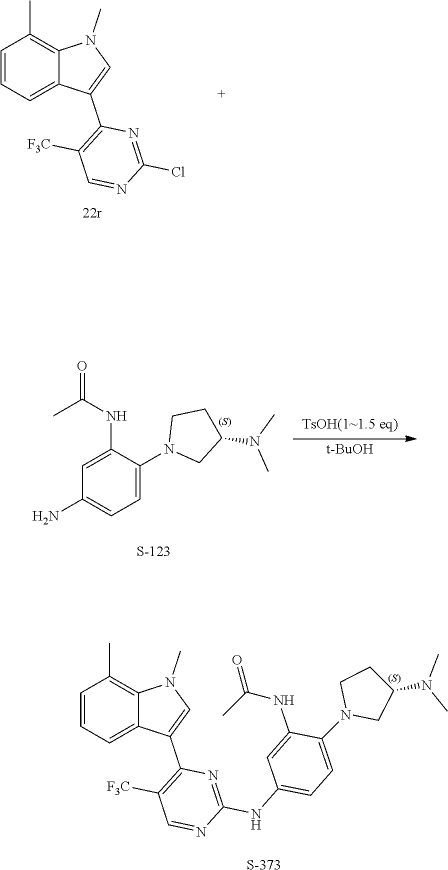

### (S)—N-(5-((4-(1,7-dimethyl-1H-indol-3-yl)-5-(trifluoromethyl) pyrimidin-2-yl)amino)-2-(3-(dimethylamino)pyrrolidin-1-yl)phenyl)acetamide (S-373)

Synthesis of S-373 was similar to that of R-373. The residue was purified by preparative thin layer chromatography (dichloromethane/methanol=12/1) to give the title product S-373 (85 mg, yield 25.1%) as an off-white solid. MS-ESI (M+H)⁺: 552.3. ¹H NMR (500 MHz, DMSO-d₆) δ 9.93 (s, 1H), 9.19 (s, 1H), 8.69 (s, 1H), 8.10 (s, 1H), 7.89 (s, 1H), 7.73 (s, 1H), 7.53 (d, J=7.0 Hz, 1H), 7.04-6.98 (m, 1H), 6.96 (d, J=6.9 Hz, 1H), 6.90 (d, J=4.1 Hz, 1H), 4.17 (s, 3H), 3.30 (s, 2H), 3.24-3.14 (m, 3H), 3.10 (s, 1H), 2.77 (s, 3H), 2.54 (s, 4H), 2.20 (s, 1H), 2.07 (s, 3H), 2.00 (s, 1H).

### Procedure for the Synthesis of 374

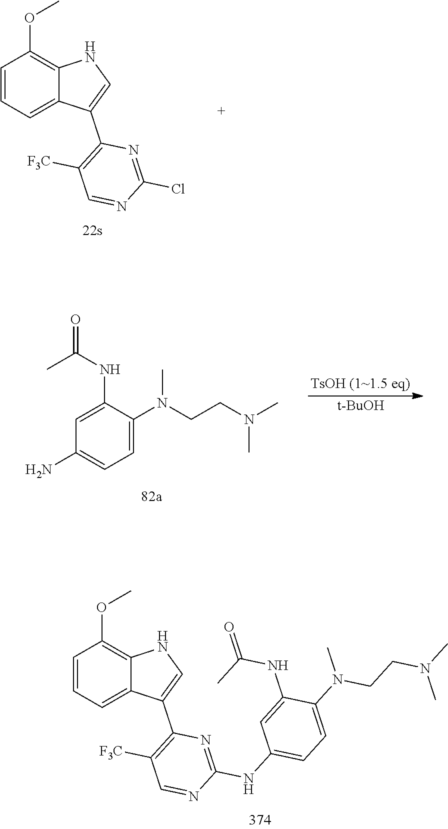

### N-(2-((2-(dimethylamino)ethyl)(methyl)amino)-5-((4-(7-methoxy-1H-indol-3-yl)-5-(trifluoromethyl)pyrimidin-2-yl)amino)phenyl)acetamide (374)

Synthesis of 374 was similar to that of 296. The residue was purified by preparative thin layer chromatography (dichloromethane/methanol=12/1) to give the title product 374 (47 mg, yield 16.9%) as a dark brown solid. MS-ESI (M+H)⁺: 542.6. ¹H NMR (500 MHz, DMSO-d₆) δ 12.03 (s, 1H), 10.00 (s, 1H), 9.85 (s, 1H), 8.70 (s, 1H), 8.38 (s, 1H), 7.90 (s, 1H), 7.73 (s, 1H), 7.56 (dd, J=8.6, 2.3 Hz, 1H), 7.19 (d, J=8.6 Hz, 1H), 7.03 (s, 1H), 6.79 (d, J=7.7 Hz, 1H), 3.95 (s, 3H), 2.94 (s, 2H), 2.62 (s, 5H), 2.41 (s, 6H), 2.13 (s, 3H).

### Procedure for the Synthesis of 375

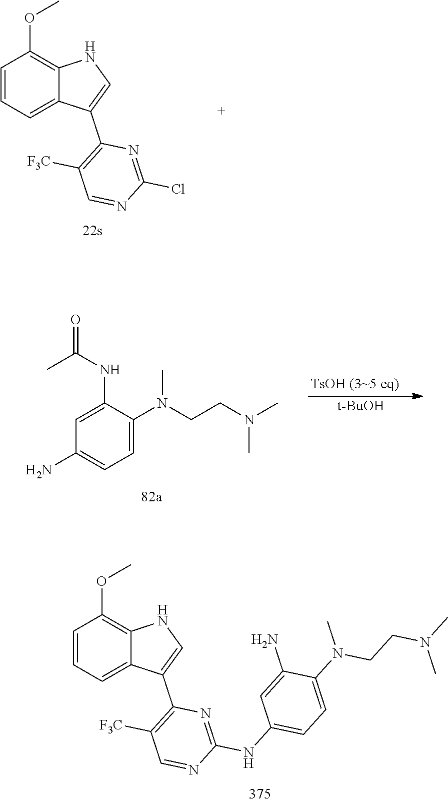

### N¹-(2-(dimethylamino)ethyl)-N⁴-(4-(7-methoxy-1H-indol-3-yl)-5-(trifluoromethyl)pyrimidin-2-yl)-N¹-methylbenzene-1,2,4-triamine (375)

Synthesis of 375 was similar to that of 297. The residue was purified by preparative thin layer chromatography (dichloromethane/methanol=12/1) to give the title product 375 (37 mg, yield 14.6%) as a dark brown solid. MS-ESI (M+H)⁺: 500.6. ¹H NMR (500 MHz, DMSO-d₆) δ 12.01 (s, 1H), 9.76 (s, 1H), 8.67 (s, 1H), 7.90 (s, 1H), 7.71 (s, 1H), 7.09-7.00 (m, 2H), 6.97 (d, J=8.0 Hz, 1H), 6.90 (d, J=8.2 Hz, 1H), 6.79 (d, J=7.6 Hz, 1H), 3.96 (s, 3H), 3.09 (s, 4H), 2.66 (s, 6H), 2.51 (s, 3H).

### Procedure for the Synthesis of 378

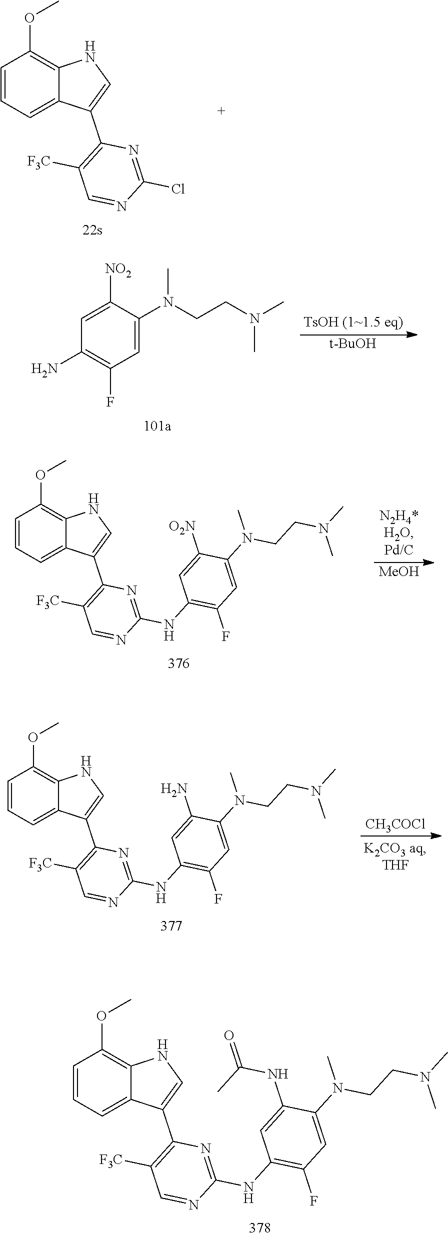

### N¹-(2-(dimethylamino)ethyl)-5-fluoro-N⁴-(4-(7-methoxy-1H-indol-3-yl)-5-(trifluoromethyl)pyrimidin-2-yl)-N¹-methyl-2-nitrobenzene-1,4-diamine (376)

Synthesis of 376 was similar to that of 296 by using 22s (153 mg, 0.47 mmol) and 101a (113 mg, 0.44 mmol) to afford the title compound 376 (84 mg, yield 34.9%).

### N¹-(2-(dimethylamino)ethyl)-5-fluoro-N⁴-(4-(7-methoxy-1H-indol-3-yl)-5-(trifluoromethyl)pyrimidin-2-yl)-N¹-methylbenzene-1,2,4-triamine (377)

A mixture of above compound 376 (84 mg, 0.15 mmol), Pd/C (5% on activated carbon, 59 mg, 0.03 mmol) and hydrazine hydrate (80%, 2 ml, 32 mmol) in methanol (10 ml) was heated at refluxing temperature for 3 hrs. After cooling to room temperature, the reaction mixtures was filtered through Celite® and the filtrates were concentrated to remove methanol. The residual was extracted with ethyl acetate. The organic layer was separated, washed with water, dried over Na₂SO₄, filtered and concentrated to dryness. The crude title compound 377 (73 mg) was directly used in the next step without further purification. LC-MS: m/z 518.2 [M+H]⁺.

### N-(2-((2-(dimethylamino)ethyl)(methyl)amino)-4-fluoro-5-((4-(7-methoxy-1H-indol-3-yl)-5-(trifluoromethyl)pyrimidin-2-yl)amino)phenyl)acetamide (378)

Acetyl chloride (30 mg, 0.38 mmol) was dropped to the mixture of 377 (73 mg, 0.14 mmol) and saturated K₂CO₃ aqueous (0.5 ml) in tetrahydrofuran (5 mL) at 0° C. The mixture was stirred for 1 hr. Then the mixture was concentrated and diluted with ethyl acetate (50 ml). The organic layers were combined, washed with brine (50 ml*3), and then concentrated under reduced pressure. The residue was purified by preparative thin layer chromatography (dichloromethane/methanol=100/7) to give the title compound 378 (26 mg, yield 30.3% over two steps) as a brown solid. LC-MS: m/z 560.2 [M+H]⁺.

### Procedure for the Synthesis of 379

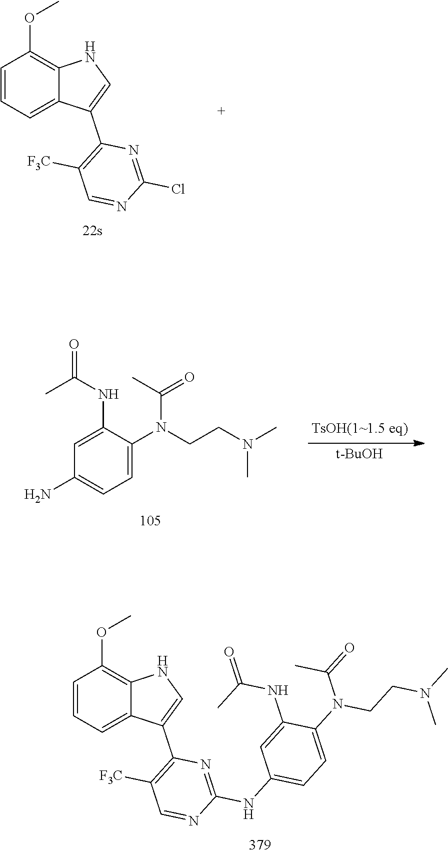

### N-(2-acetamido-4-((4-(7-methoxy-1H-indol-3-yl)-5-(trifluoromethyl) pyrimidin-2-yl)amino)phenyl)-N-(2-(dimethylamino)ethyl)acetamide (379)

Synthesis of 379 was similar to that of 296. The residue was purified by preparative thin layer chromatography (dichloromethane/methanol=12/1) to give the title product 379 (20 mg, yield 6.9%) as a light brown solid. MS-ESI (M+Na)⁺: 592.3. ¹H NMR (500 MHz, DMSO-d) δ 12.01 (s, 1H), 10.76 (s, 1H), 10.20 (s, 1H), 8.77 (s, 1H), 8.30 (s, 1H), 7.91 (s, 1H), 7.81-7.70 (m, 2H), 7.23 (d, J=8.6 Hz, 1H), 7.05 (t, J=7.7 Hz, 1H), 6.80 (d, J=7.6 Hz, 1H), 4.50 (s, 1H), 3.96 (s, 3H), 3.13 (s, 1H), 2.35 (d, J=28.4 Hz, 2H), 2.25 (s, 6H), 2.02 (s, 3H), 1.61 (s, 3H).

### Procedure for the Synthesis of 380

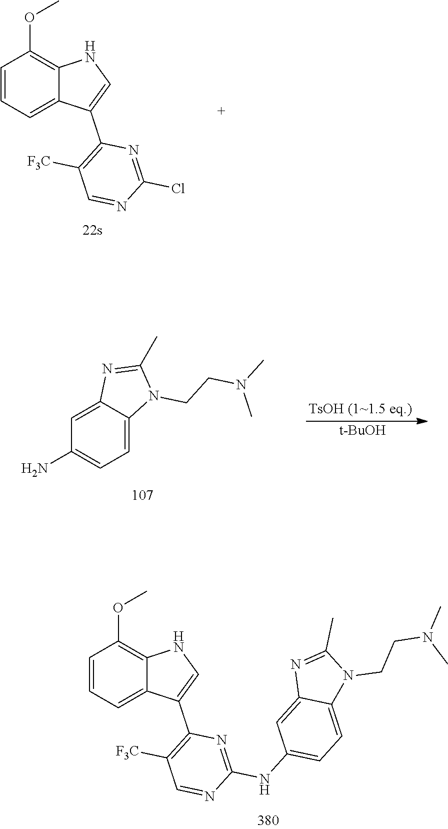

### 1-(2-(dimethylamino)ethyl)-N-(4-(7-methoxy-1H-indol-3-yl)-5-(trifluoromethyl)pyrimidin-2-yl)-2-methyl-1H-benzo[d]imidazol-5-amine (380)

Synthesis of 380 was similar to that of 296. The residue was purified by preparative thin layer chromatography (dichloromethane/methanol=12/1) to give the title product 380 (3 mg, yield 1.0%) as a yellow solid. MS-ESI (M+H)⁺: 510.4. ¹H NMR (500 MHz, DMSO-d₆) δ 12.01 (s, 1H), 9.97 (s, 1H), 8.71 (s, 1H), 7.94 (s, 1H), 7.71 (s, 1H), 7.52 (dd, J=8.7, 1.8 Hz, 1H), 7.41 (d, J=8.8 Hz, 1H), 6.99 (s, 1H), 6.77 (d, J=7.5 Hz, 1H), 4.25 (t, J=6.5 Hz, 2H), 3.95 (s, 3H), 2.62 (s, 2H), 2.53 (s, 3H), 2.25 (s, 4H).

### Procedure for the Synthesis of 381

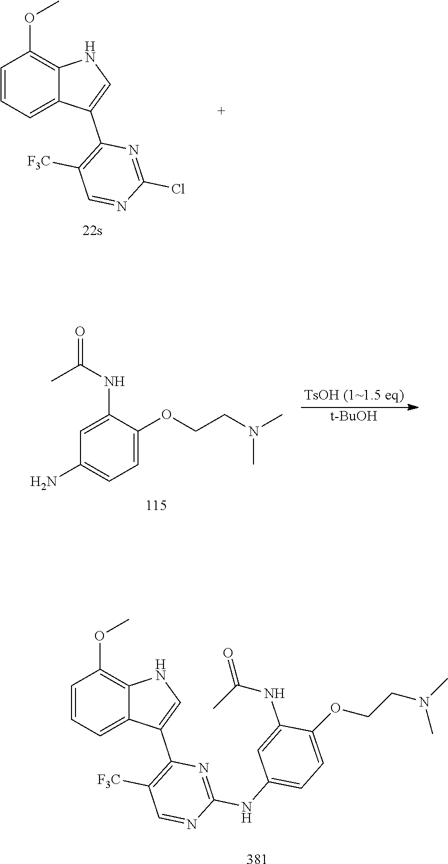

### N-(2-(2-(dimethylamino)ethoxy)-5-((4-(7-methoxy-1H-indol-3-yl)-5-(trifluoromethyl)pyrimidin-2-yl)amino)phenyl)acetamide (381)

Synthesis of 381 was similar to that of 296. The residue was purified by preparative thin layer chromatography (dichloromethane/methanol=12/1) to give the title product 381 (11 mg, yield 3.4%) as a brown yellow solid. MS-ESI (M+H)⁺: 529.5. ¹H NMR (500 MHz, DMSO-d₆) δ 12.03 (s, 1H), 9.96 (s, 1H), 9.64 (s, 1H), 8.70 (s, 1H), 8.28 (s, 1H), 7.88 (s, 1H), 7.72 (s, 1H), 7.52 (d, J=7.2 Hz, 1H), 7.04 (d, J=8.6 Hz, 2H), 6.79 (d, J=7.7 Hz, 1H), 4.12 (t, J=5.3 Hz, 2H), 3.96 (s, 3H), 2.70 (s, 2H), 2.35 (s, 6H), 2.08 (s, 3H).

### Procedure for the Synthesis of R-382

### (R)—N-(2-(3-(dimethylamino)pyrrolidin-1-yl)-5-((4-(7-methoxy-1H-indol-3-yl)-5-(trifluoromethyl)pyrimidin-2-yl)amino)phenyl)acetamide (R-382)

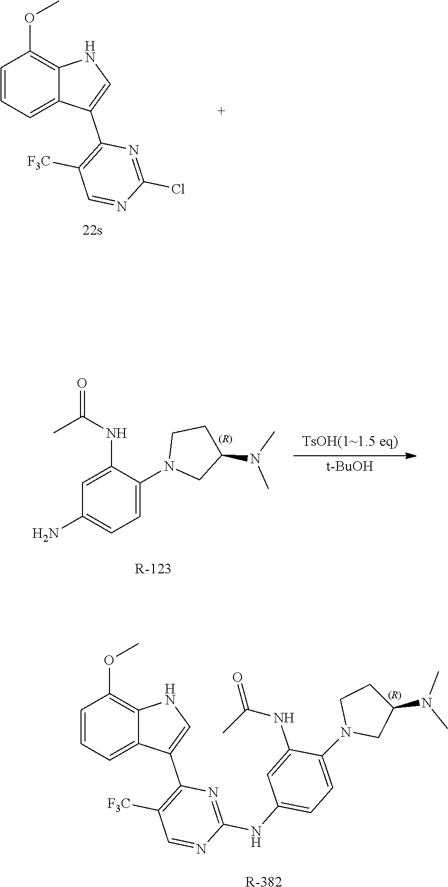

A mixture of 22s (0.22 g, 0.67 mmol), R-123 (0.16 g, 0.61 mmol), and TsOH (0.12 g, 0.70 mmol) in t-BuOH (15 mL) was stirred at 100° C. overnight. After cooling to room temperature, the mixture was neutralized with saturated NaHCO₃ and concentrated under reduced pressure. The residue was dissolved in dichloromethane (50 mL), and washed with H₂O (20 mL*2), dried with Na₂SO₄. After concentration, the residue was purified by preparative thin layer chromatography (dichloromethane/methanol=12/1) to give the desired product R-382 (18 mg, yield 5.3%) as a dark brown solid. MS-ESI (M+H)⁺: 554.7. ¹H NMR (500 MHz, DMSO-d₆) δ 12.03 (s, 1H), 9.96 (s, 1H), 9.22 (s, 1H), 8.69 (s, 1H), 7.98 (s, 1H), 7.71 (s, 1H), 7.60-7.53 (m, 1H), 7.06 (t, J=7.1 Hz, 1H), 6.97 (d, J=8.3 Hz, 1H), 6.79 (d, J=7.7 Hz, 1H), 3.96 (s, 3H), 3.28 (s, 2H), 3.19 (dd, J=10.6, 7.0 Hz, 1H), 3.00 (d, J=6.7 Hz, 1H), 2.75 (s, 6H), 2.28 (s, 1H), 2.17 (s, 1H), 2.12 (s, 3H).

### Procedure for the Synthesis of R-383

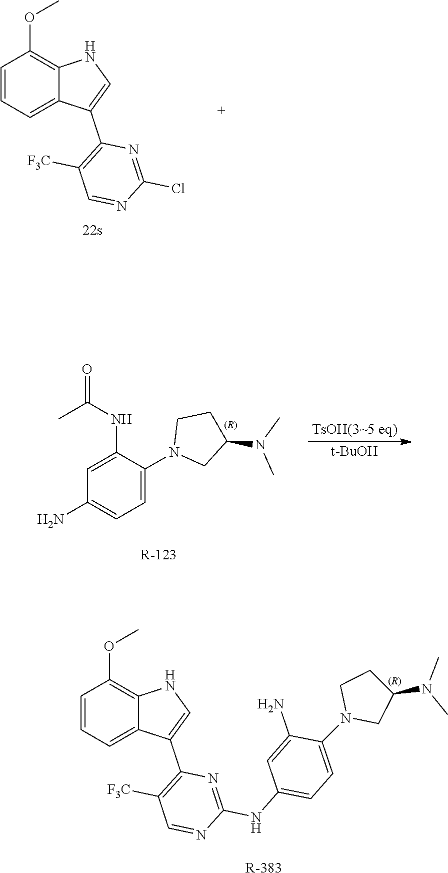

### (R)-4-(3-(dimethylamino)pyrrolidin-1-yl)-N¹-(4-(7-methoxy-1H-indol-3-yl)-5-(trifluoromethyl)pyrimidin-2-yl)benzene-1,3-diamine (R-383)

Synthesis of R-383 was similar to that of 297. The residue was purified by preparative thin layer chromatography (dichloromethane/methanol=12/1) to give the title product R-383 (22 mg, yield 11.3%) as a brown solid. MS-ESI (M+H)⁺: 512.5. ¹H NMR (500 MHz, DMSO-d₆) δ 11.98 (s, 1H), 9.73 (s, 1H), 8.66 (s, 1H), 7.89 (s, 1H), 7.71 (s, 1H), 7.05 (s, 2H), 7.01-6.92 (m, 1H), 6.84 (d, J=6.0 Hz, 1H), 6.82-6.72 (m, 1H), 4.81 (s, 2H), 3.96 (s, 3H), 3.71 (s, 1H), 3.18-3.06 (m, 3H), 2.92-2.81 (m, 1H), 2.70 (s, 7H), 2.24 (s, 1H), 2.08 (s, 1H).

### Procedure for the Synthesis of S-382a and S-382b

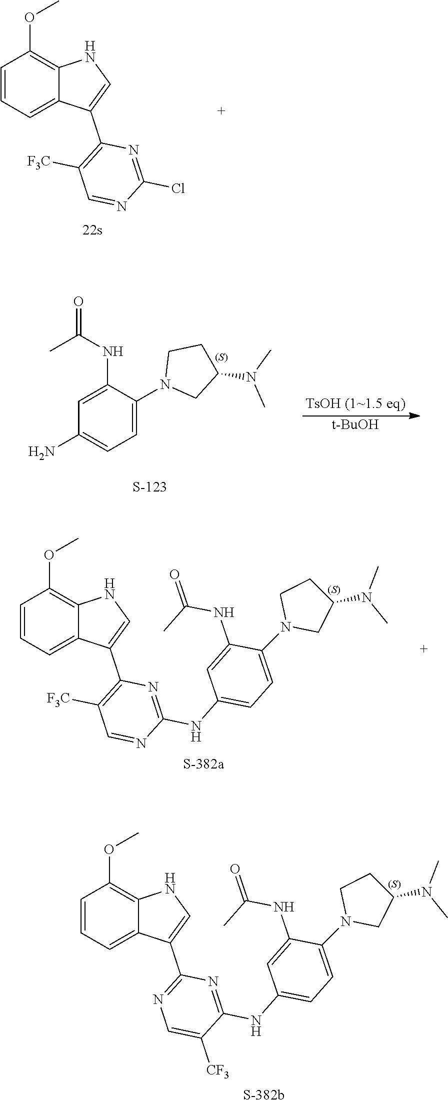

### (S)—N-(2-(3-(dimethylamino)pyrrolidin-1-yl)-5-((4-(7-methoxy-1H-indol-3-yl)-5-(trifluoromethyl)pyrimidin-2-yl)amino)phenyl)acetamide (S-382a) & (S)—N-(2-(3-(dimethylamino)pyrrolidin-1-yl)-5-((2-(7-methoxy-1H-indol-3-yl)-5-(trifluoromethyl)pyrimidin-4-yl)amino)phenyl)acetamide (S-382b)

Synthesis of S-382a and S-382b were similar to that of R-382. The residue was purified by preparative thin layer chromatography (dichloromethane/methanol=12/1) to give the title product S-382a (37 mg, yield 11.0%) as an orangey solid and products S-382b (11 mg, yield 3.3%) as an light yellow solid.

For S-382a: MS-ESI (M+H)⁺: 554.7.

For S-382b: MS-ESI (M+H)⁺: 554.5. ¹H NMR (500 MHz, DMSO-d₆) δ 11.86 (s, 1H), 9.27 (s, 1H), 8.73 (s, 1H), 8.56 (s, 1H), 8.03 (d, J=2.3 Hz, 1H), 7.82 (s, 1H), 7.68 (d, J=8.0 Hz, 1H), 7.24 (d, J=7.4 Hz, 1H), 7.03 (d, J=8.5 Hz, 1H), 6.93 (t, J=7.8 Hz, 1H), 6.71 (d, J=7.7 Hz, 1H), 3.92 (s, 3H), 3.29-3.21 (m, 5H), 2.51 (s, 6H), 2.24 (s, 1H), 2.11 (s, 3H), 2.00 (dq, J=14.0, 6.5 Hz, 1H).

### Procedure for the Synthesis of S-383

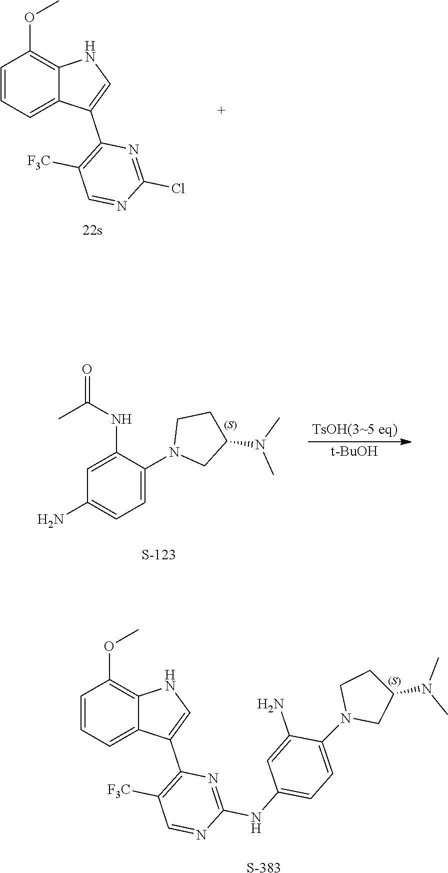

### (S)-4-(3-(dimethylamino)pyrrolidin-1-yl)-N¹-(4-(7-methoxy-1-1H-indol-3-yl)-5-(trifluoromethyl)pyrimidin-2-yl)benzene-1,3-diamine (S-383)

Synthesis of S-383 was similar to that of R-383. The residue was purified by preparative thin layer chromatography (dichloromethane/methanol=12/1) to give the title product S-383 (33 mg, yield 16.9%) as a brown solid. MS-ESI (M+H)⁺: 512.4. ¹H NMR (500 MHz, DMSO-d₆) δ 11.99 (s, 1H), 9.74 (s, 1H), 8.67 (s, 1H), 7.90 (s, 1H), 7.71 (s, 1H), 7.05 (d, J=10.9 Hz, 2H), 6.98 (d, J=7.7 Hz, 1H), 6.85 (d, J=8.3 Hz, 1H), 6.79 (d, J=7.7 Hz, 1H), 4.83 (s, 2H), 3.96 (s, 3H), 3.81 (s, 1H), 3.23-3.15 (m, 2H), 3.14-3.05 (m, 1H), 2.83 (q, J=7.3 Hz, 1H), 2.75 (s, 6H), 2.32-2.21 (m, 1H), 2.12 (dq, J=13.2, 6.4 Hz, 1H).

### Procedure for the Synthesis of 384

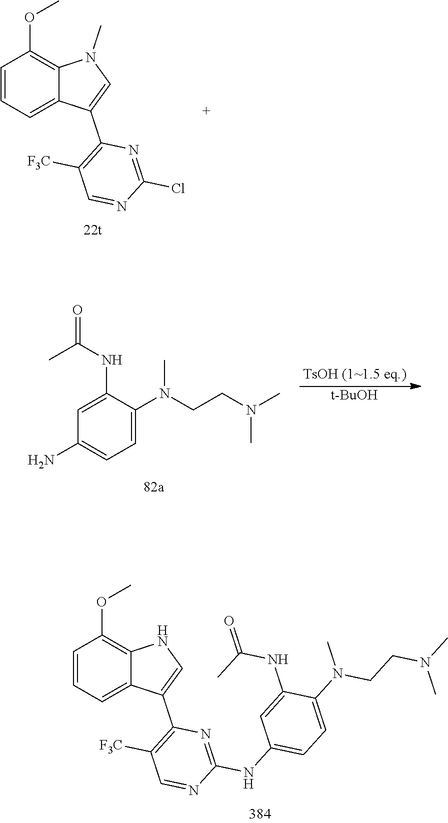

### N-(2-((2-(dimethylamino)ethyl)(methyl)amino)-5-((4-(7-methoxy-1-methyl-1H-indol-3-yl)-5-(trifluoromethyl)pyrimidin-2-yl)amino)phenyl)acetamide (384)

Synthesis of 384 was similar to that of 296. The residue was purified by preparative thin layer chromatography (dichloromethane/methanol=12/1) to give the title product 384 (48 mg, yield 14.1%) as a light yellow solid. MS-ESI (M+H)⁺: 556.5. ¹H NMR (500 MHz, DMSO-d₆) δ 10.02 (s, 1H), 9.83 (s, 1H), 8.70 (s, 1H), 8.37 (s, 1H), 7.87 (s, 1H), 7.71 (s, 1H), 7.55 (dd, J=8.7, 2.4 Hz, 1H), 7.18 (d, J=8.6 Hz, 1H), 7.03 (t, J=7.0 Hz, 1H), 6.78 (d, J=7.8 Hz, 1H), 4.10 (s, 3H), 3.91 (s, 3H), 2.97 (s, 2H), 2.71 (s, 1H), 2.61 (s, 3H), 2.45 (s, 6H), 2.15 (s, 3H).

### Procedure for the Synthesis of 385

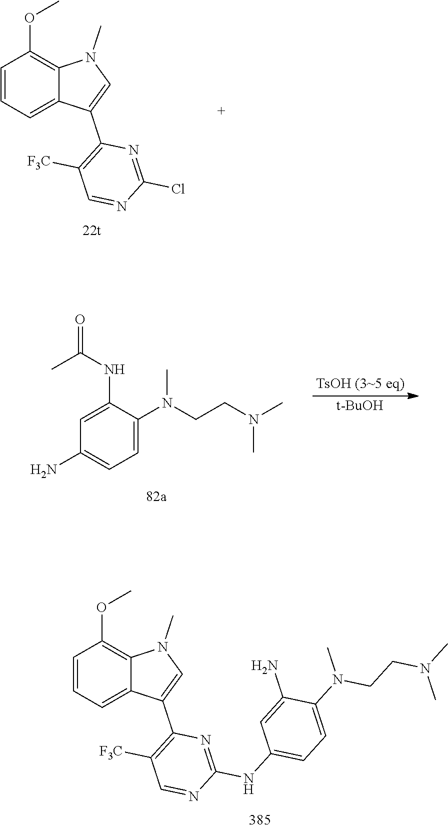

### N¹-(2-(dimethylamino)ethyl)-N⁴-(4-(7-methoxy-1-methyl-1H-indol-3-yl)-5-(trifluoromethyl)pyrimidin-2-yl)-N¹-methylbenzene-1,2,4-triamine (385)

Synthesis of 385 was similar to that of 297. The residue was purified by preparative thin layer chromatography (dichloromethane/methanol=12/1) to give the title product 385 (55 mg, yield 18.3%) as a light brown solid. MS-ESI (M+H)⁺: 514.6. ¹H NMR (500 MHz, DMSO-d₆) δ 9.77 (s, 1H), 8.67 (s, 1H), 7.87 (s, 1H), 7.69 (s, 1H), 7.08-6.99 (m, 2H), 6.96 (d, J=8.4 Hz, 1H), 6.88 (d, J=8.1 Hz, 1H), 6.78 (d, J=7.6 Hz, 1H), 4.10 (s, 3H), 3.91 (s, 3H), 3.05 (s, 4H), 2.61 (s, 6H), 2.51 (s, 3H).

### Procedure for the Synthesis of 386

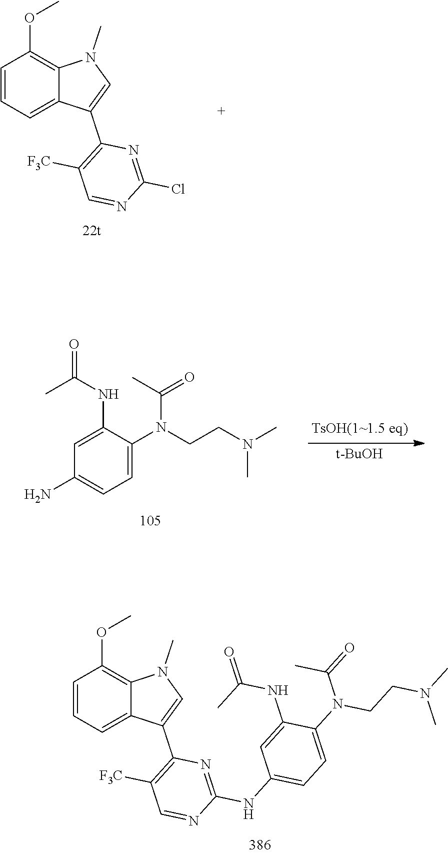

### N-(2-acetamido-4-((4-(7-methoxy-1-methyl-1H-indol-3-yl)-5-(trifluoromethyl)pyrimidin-2-yl)amino)phenyl)-N-(2-(dimethylamino)ethyl)acetamide (386)

Synthesis of 386 was similar to that of 296. The residue was purified by preparative thin layer chromatography (dichloromethane/methanol=12/1) to give the title product 386 (47 mg, yield 13.8%) as a light yellow solid. MS-ESI (M+Na)⁺: 606.5. ¹H NMR (500 MHz, Chloroform-d) δ 11.01 (s, 1H), 8.69 (s, 1H), 8.23 (s, 1H), 8.00-7.93 (m, 1H), 7.90 (d, J=7.7 Hz, 1H), 7.81 (s, 1H), 7.54 (s, 1H), 7.07 (t, J=9.8 Hz, 2H), 6.69 (d, J=7.3 Hz, 1H), 4.99-4.86 (m, 1H), 4.12 (s, 3H), 3.94 (s, 3H), 2.91 (d, J=13.9 Hz, 1H), 2.43 (d, J=16.0 Hz, 2H), 2.31 (s, 6H), 2.11 (s, 3H), 1.76 (s, 3H).

### Procedure for the Synthesis of 387

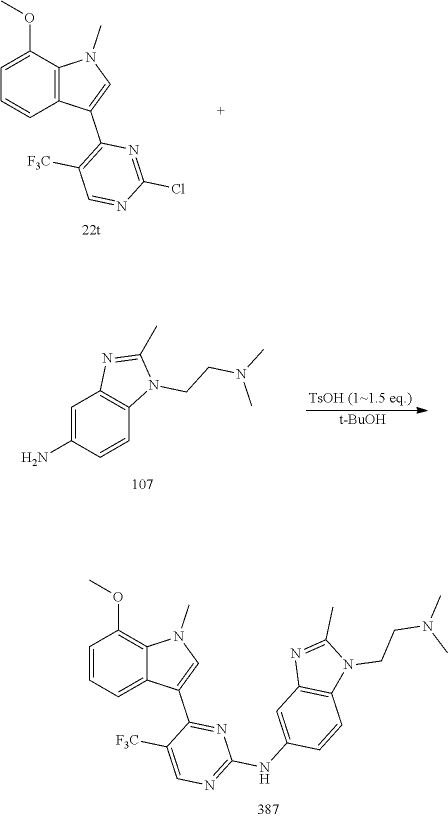

### 1-(2-(dimethylamino)ethyl)-N-(4-(7-methoxy-1-methyl-1H-indol-3-yl)-5-(trifluoromethyl)pyrimidin-2-yl)-2-methyl-1H-benzo[d]imidazol-5-amine (387)

To the mixture of 22t (211 mg, 0.62 mmol) and 107 (131 mg, 0.60 mmol) in t-BuOH (10 mL) was added TsOH (116 mg, 0.60 mmol). The mixture was stirred at 80° C. for 24 h. The mixture was concentrated and the residue was purified by preparative thin layer chromatography. The product was basified by aqueous NaHCO₃ to give 387 (17 mg, 0.032 mmol, 5.4% yield) as off-white solid. MS-ESI (M+H⁺): 524.4; ¹H NMR (500 MHz, DMSO-d₆) δ 9.97 (s, 1H), 8.70 (s, 1H), 7.94 (s, 1H), 7.69 (s, 1H), 7.51 (dd, J=8.6, 1.7 Hz, 1H), 7.39 (d, J=8.5 Hz, 1H), 6.97 (s, 1H), 6.76 (d, J=7.5 Hz, 1H), 4.23 (t, J=6.5 Hz, 2H), 4.10 (s, 3H), 3.91 (s, 3H), 2.58 (t, J=5.6 Hz, 2H), 2.52 (s, 3H), 2.22 (s, 6H).

### Procedure for the Synthesis of 388

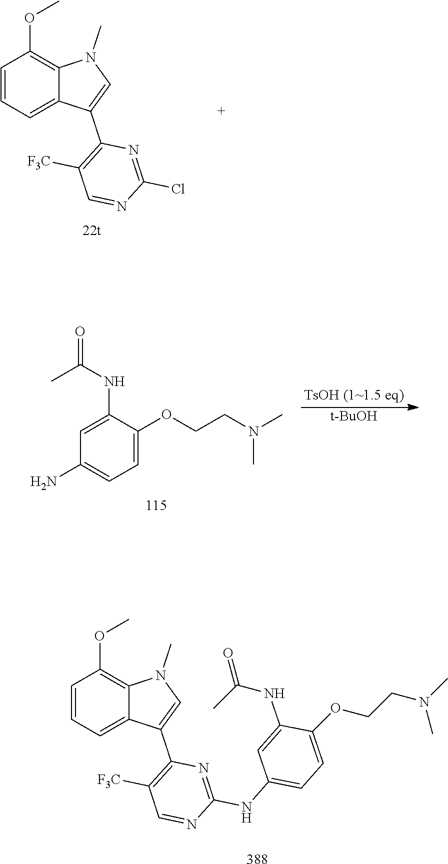

### N-(2-(2-(dimethylamino)ethoxy)-5-((4-(7-methoxy-1-methyl-1H-indol-3-yl)-5-(trifluoromethyl)pyrimidin-2-yl)amino)phenyl)acetamide (388)

Synthesis of 388 was similar to that of 296. The residue was purified by preparative thin layer chromatography (dichloromethane/methanol=12/1) to give the title product 388 (24 mg, yield 8.1%) as a light brown solid. MS-ESI (M+H)⁺: 543.4. ¹H NMR (500 MHz, DMSO-d₆) δ 9.96 (s, 1H), 9.62 (s, 1H), 8.69 (s, 1H), 8.28 (s, 1H), 7.71 (s, 1H), 7.55-7.48 (m, 1H), 7.02 (d, J=8.4 Hz, 2H), 6.78 (d, J=7.7 Hz, 1H), 4.17-4.12 (m, 2H), 4.10 (s, 3H), 3.91 (s, 3H), 2.80 (s, 2H), 2.41 (s, 6H), 2.09 (s, 3H).

### Procedure for the Synthesis of R-389

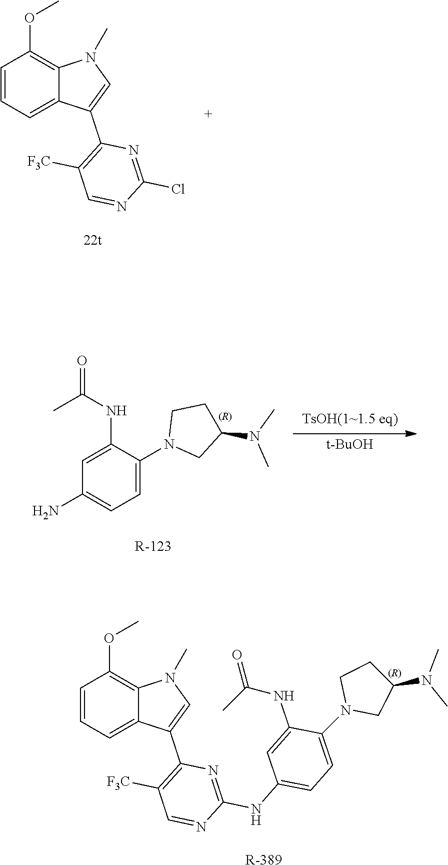

### (R)—N-(2-(3-(dimethylamino)pyrrolidin-1-yl)-5-((4-(7-methoxy-1-methyl-1H-indol-3-yl)-5-(trifluoromethyl)pyrimidin-2-yl)amino)phenyl)acetamide (R-389)

Synthesis of R-389 was similar to that of 296. The residue was purified by preparative thin layer chromatography (dichloromethane/methanol=12/1) to give the title product R-389 (83 mg, yield 25.7%) as a light yellow solid. MS-ESI (M+H)⁺: 568.4. ¹H NMR (500 MHz, DMSO-d₆) δ 9.94 (s, 1H), 9.24 (s, 1H), 8.69 (s, 1H), 7.91 (s, 1H), 7.70 (s, 1H), 7.58-7.49 (m, 1H), 7.05 (t, J=7.4 Hz, 1H), 6.94-6.85 (m, 1H), 6.78 (d, J=7.8 Hz, 1H), 4.10 (s, 3H), 3.91 (s, 3H), 3.33 (s, 2H), 3.21 (s, 2H), 3.17 (d, J=8.2 Hz, 2H), 3.12-3.03 (m, 1H), 2.51 (s, 6H), 2.19 (s, 1H), 2.10 (s, 3H), 2.00 (s, 1H).

### Procedure for the Synthesis of S-389

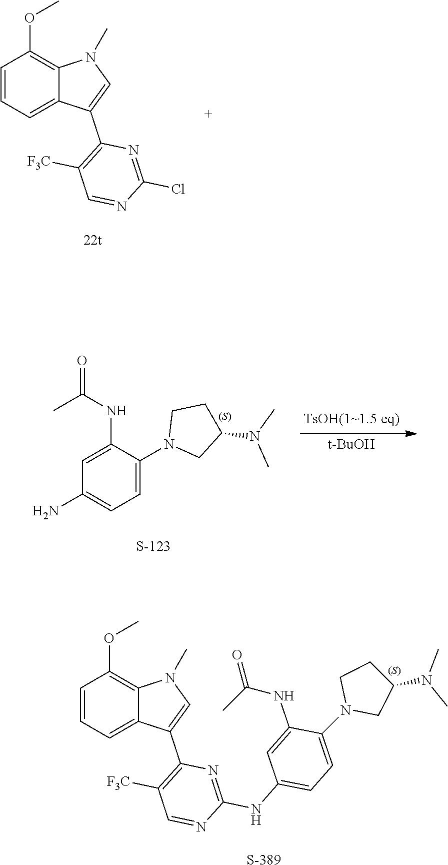

### (S)—N-(2-(3-(dimethylamino)pyrrolidin-1-yl)-5-((4-(7-methoxy-1-methyl-1H-indol-3-yl)-5-(trifluoromethyl)pyrimidin-2-yl)amino)phenyl)acetamide (S-389)

Synthesis of S-389 was similar to that of R-389. The residue was purified by preparative thin layer chromatography (dichloromethane/methanol=12/1) to give the title product S-389 (94 mg, yield 28.3%) as a light yellow solid. MS-ESI (M+H)⁺: 568.6. ¹H NMR (500 MHz, DMSO-d₆) δ 9.93 (s, 1H), 9.21 (s, 1H), 8.69 (s, 1H), 7.87 (s, 1H), 7.70 (s, 1H), 7.53 (d, J=7.8 Hz, 1H), 7.05 (t, J=6.5 Hz, 1H), 6.95-6.83 (m, 1H), 6.78 (d, J=7.7 Hz, 1H), 4.10 (s, 3H), 3.92 (s, 3H), 3.23-3.07 (m, 5H), 2.48 (s, 6H), 2.17 (s, 1H), 2.08 (s, 3H), 1.97 (s, 1H).

### Procedure for the Synthesis of S-390

### (S)-4-(3-(dimethylamino)pyrrolidin-1-yl)-N¹-(4-(7-methoxy-1-methyl-1H-indol-3-yl)-5-(trifluoromethyl)pyrimidin-2-yl)benzene-1,3-diamine (S-390)

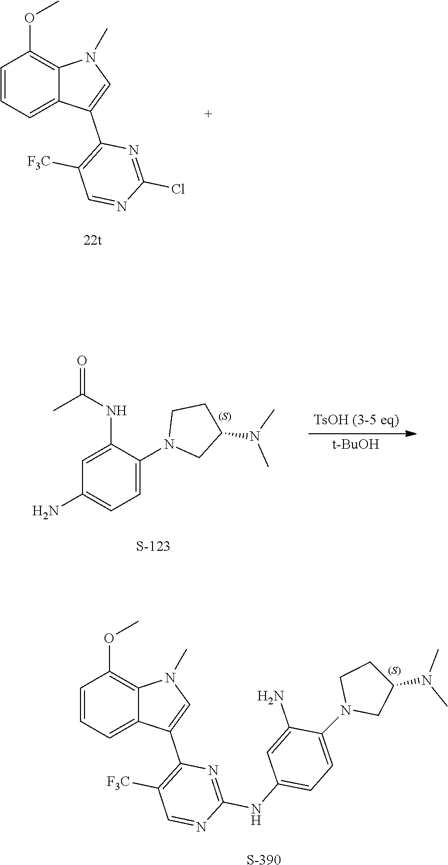

Synthesis of S-390 was similar to that of 297. The residue was purified by preparative thin layer chromatography (dichloromethane/methanol=12/1) to give the title product S-390 (79 mg, yield 36.0%) as a brown solid. MS-ESI (M+H)⁺: 526.4. ¹H NMR (500 MHz, DMSO-d₆) δ 9.74 (s, 1H), 8.66 (s, 1H), 7.87 (s, 1H), 7.68 (s, 1H), 7.04 (d, J=10.3 Hz, 2H), 6.97 (d, J=8.1 Hz, 1H), 6.83 (d, J=8.2 Hz, 1H), 6.78 (d, J=7.7 Hz, 1H), 4.82 (s, 2H), 4.10 (s, 3H), 3.92 (s, 3H), 3.77 (s, 1H), 3.18 (s, 2H), 3.13-3.06 (m, 1H), 2.84 (q, J=7.4 Hz, 1H), 2.73 (s, 6H), 2.31-2.18 (m, 1H), 2.15-2.04 (m, 1H).

### Procedure for the Synthesis of 391

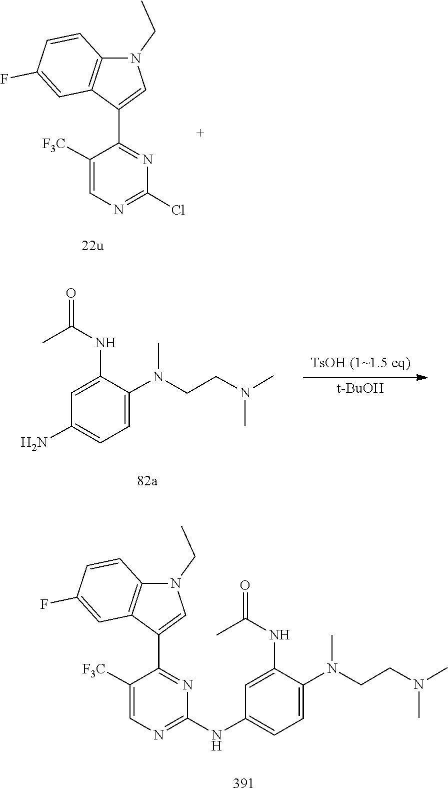

### N-(2-((2-(dimethylamino)ethyl)(methyl)amino)-5-((4-(1-ethyl-5-fluoro-1H-indol-3-yl)-5-(trifluoromethyl)pyrimidin-2-yl)amino)phenyl)acetamide (391)

A mixture of 22u (198 mg, 0.58 mmol), 82a (151 mg, 0.60 mmol) and TsOH (106 mg, 0.062 mmol) in t-BuOH (10 mL) was stirred at 80° C. for 22 h. The reaction was then cooled to room temperature and diluted with dichloromethane (100 mL). Then the resulting solution was washed with saturated NaHCO₃, brine. The organic layer was dried over Na₂SO₄, filtered and concentrated under reduced pressure to give a residue. The residue was purified by flash column chromatography (dichloromethane/methanol=20/1-10/1, 1% Et₃N) to afford 391 (78 mg, yield 24.3%) as slight yellow solid. ¹H NMR (500 MHz, CDCl₃) δ 10.04 (s, 1H), 8.65 (s, 1H), 8.48 (s, 1H), 8.06 (dd, J=10.4, 2.4 Hz, 1H), 7.82 (s, 1H), 7.63 (d, J=6.5 Hz, 1H), 7.39 (s, 1H), 7.29 (dd, J=8.9, 4.4 Hz, 1H), 7.20 (d, J=8.6 Hz, 1H), 7.02 (td, J=8.9, 2.5 Hz, 1H), 4.23 (q, J=7.3 Hz, 2H), 2.90 (m, 2H), 2.70 (s, 3H), 2.33 (m, 8H), 2.20 (s, 3H), 1.52 (t, J=7.3 Hz, 3H). MS-ESI (M+H)⁺: 558.5.

### Procedure for the Synthesis of 392

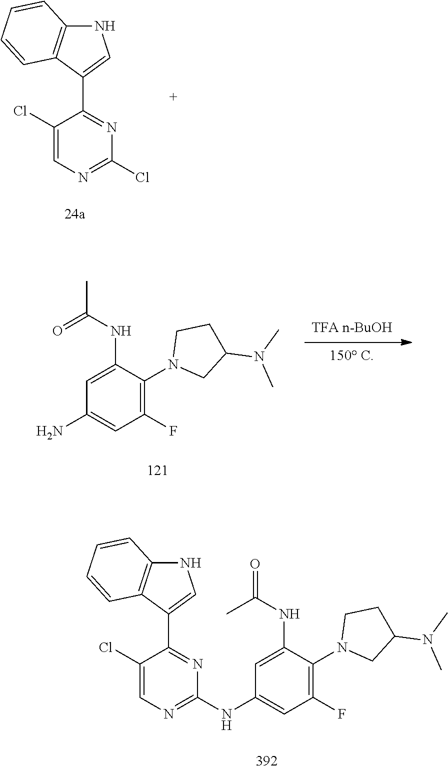 [SMILES: Clc1ncc(Cl)c(-c2c[nH]c3ccccc23)n1 ; CC(=O)Nc1cc(N)cc(F)c1N1CCC(N(C)C)C1 ; CC(=O)Nc1cc(Nc2ncc(Cl)c(-c3c[nH]c4ccccc34)n2)cc(F)c1N1CCC(N(C)C)C1]

### N-(5-((5-chloro-4-(1H-indol-3-yl)pyrimidin-2-yl)amino)-2-(3-(dimethylamino)pyrrolidin-1-yl)-3-fluorophenyl)acetamide (392)

To a mixture of 121 (300.0 mg, 1.7 mmol) and 24a (475.0 mg, 1.8 mmol) in n-BuOH (30 mL) was added TFA (0.1 mL). The mixture was allowed to stir at 150° C. under Ar for 30 h. The reaction mixture was concentrated under vacuum to afford crude 392, which was purified with prep-HPLC (MeCN/0.05% formic acid in water from 0 to 100%) to give the title product 392 as a brown solid (35.2 mg, yield 4.7%). MS-ESI (M+H)⁺: 508.2. ¹H NMR (400 MHz, CD3OD) δ 8.59 (d, J=8.0 Hz, 1H), 8.54 (s, 1H), 8.43 (d, J=3.2 Hz, 1H), 8.34 (s, 1H), 8.19 (s, 1H), 7.65 (dd, J=15.2, 2.4 Hz, 1H), 7.45 (d, J=8.0 Hz, 1H), 7.21 (dd, J=11.1, 4.0 Hz, 1H), 7.15 (d, J=7.2 Hz, 1H), 3.59-3.57 (m, 1H), 3.43-3.39 (m, 1H), 3.35-3.21 (m, 3H), 2.67 (s, 6H), 2.34 (m, 1H), 2.18 (s, 3H), 2.14-2.07 (m, 1H).

### Procedure for the Synthesis of 393

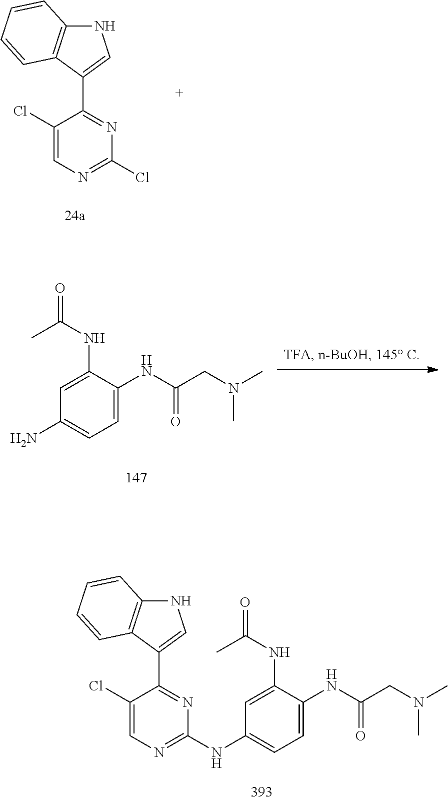 [SMILES: Clc1ncc(Cl)c(-c2c[nH]c3ccccc23)n1 ; CC(=O)Nc1cc(N)ccc1NC(=O)CN(C)C ; CC(=O)Nc1cc(Nc2ncc(Cl)c(-c3c[nH]c4ccccc34)n2)ccc1NC(=O)CN(C)C]

### N-(2-acetamido-4-((5-chloro-4-(1H-indol-3-yl)pyrimidin-2-yl)amino)phenyl)-2-(dimethylamino)acetamide (393)

The mixture of 147 (200 mg, 0.80 mmol) and 3-(2,5-dichloropyrimidin-4-yl)-1H-indole 24a (222 mg, 0.84 mmol) in n-BuOH (6 mL) was added TFA (0.12 mL, 1.60 mmol). The mixture was stirred at 145° C. under Ar for 48 h. The reaction was concentrated in vacuo and purified by Prep-HPLC (NH₃—H₂O) to give 994 (45.00 mg, 11.78% yield) as a white solid. MS-ESI (M+H⁺): 278.2. ¹H NMR (400 MHz, DMSO) δ 11.93 (s, 1H), 9.71 (s, 2H), 9.35 (s, 1H), 8.59 (d, J=7.9 Hz, 1H), 8.51 (d, J=3.0 Hz, 1H), 8.47 (s, 1H), 7.83 (s, 1H), 7.62 (d, J=8.7 Hz, 1H), 7.55 (d, J=8.9 Hz, 1H), 7.50 (s, 1H), 7.23 (t, J=7.1 Hz, 1H), 7.14 (t, J=7.4 Hz, 1H), 3.12 (s, 2H), 2.34 (s, 6H), 2.03 (s, 3H).

### Procedure for the Synthesis of 394a and 394b

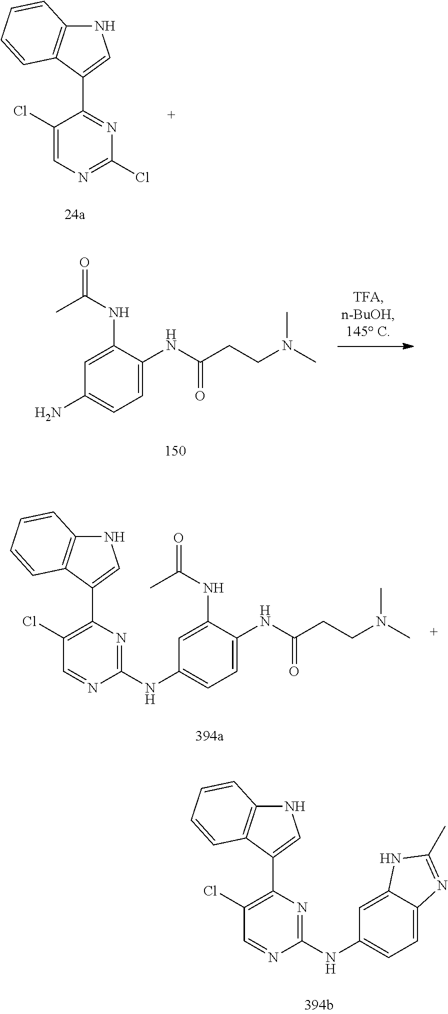 [SMILES: Clc1ncc(Cl)c(-c2c[nH]c3ccccc23)n1 ; CC(=O)Nc1cc(N)ccc1NC(=O)CCN(C)C ; CC(=O)Nc1cc(Nc2ncc(Cl)c(-c3c[nH]c4ccccc34)n2)ccc1NC(=O)CCN(C)C ; Cc1nc2ccc(Nc3ncc(Cl)c(-c4c[nH]c5ccccc45)n3)cc2[nH]1]

### N-(2-acetamido-4-((5-chloro-4-(1H-indol-3-yl)pyrimidin-2-yl)amino)phenyl)-3-(dimethylamino)propanamide (394a) & N-(5-chloro-4-(1H-indol-3-yl)pyrimidin-2-yl)-2-methyl-1H-benzo[d]imidazol-6-amine (394b)

To the mixture of 150 (200 mg, 0.76 mmol) and 3-(2,5-dichloropyrimidin-4-yl)-1H-indole 24a (210 mg, 0.79 mmol) in n-BuOH (5 mL) was added TFA (0.11 mL, 1.51 mmol). The mixture was stirred at 145° C. under Ar for 16 h. The mixture was concentrated and the residue was purified by Prep-HPLC (formic acid), then basified by aq. Na₂CO₃ to give 394a (19.90 mg, 5.35% yield) as a white solid and 394b (23.88 mg, 8.42% yield) as a white solid.

For 394a: MS-ESI (M+H⁺): 492.2. ¹H NMR (400 MHz, DMSO-d₆) δ 11.88 (s, 1H), 9.65 (s, 2H), 9.24 (s, 1H), 8.60 (d, J=7.7 Hz, 1H), 8.52 (s, 1H), 8.45 (s, 1H), 7.95 (s, 1H), 7.62-7.45 (m, 3H), 7.22 (t, J=7.4 Hz, 1H), 7.14 (t, J=7.5 Hz, 1H), 2.58-2.53 (m, 2H), 2.47-2.38 (m, 2H), 2.20 (s, 6H), 2.02 (s, 3H).

For 394b: MS-ESI (M+H⁺): 375.1. ¹H NMR (400 MHz, DMSO-d₆) δ 12.01 (s, 1H), 11.88 (s, 1H), 9.51 (s, 1H), 8.56 (s, 1H), 8.49 (d, J=2.7 Hz, 1H), 8.43 (s, 1H), 8.17 (s, 1H), 7.91 (s, 1H), 7.49 (d, J=8.1 Hz, 1H), 7.39 (s, 2H), 7.20 (t, J=7.5 Hz, 1H), 7.10-6.99 (m, 1H), 2.46 (s, 3H).

### Procedure for the Synthesis of 395

### N-(5-((5-chloro-4-(1-methyl-1H-indol-3-yl)pyrimidin-2-yl)amino)-2-((2-(dimethylamino)ethyl)(methyl)amino)phenyl)acetamide (395)

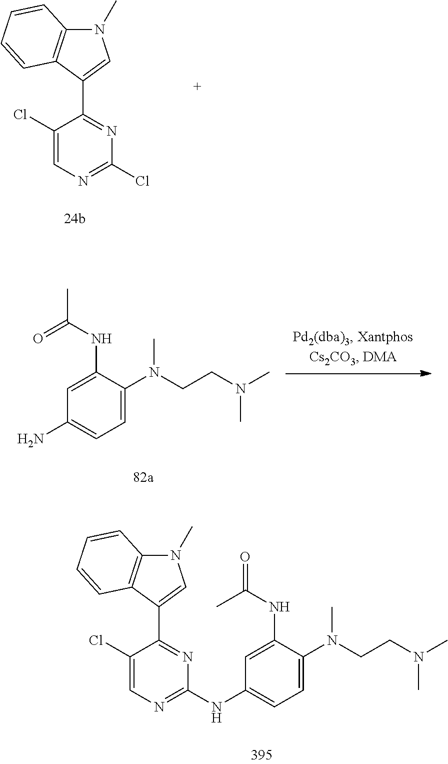 [SMILES: Cn1cc(-c2nc(Cl)ncc2Cl)c2ccccc21 ; CC(=O)Nc1cc(N)ccc1N(C)CCN(C)C ; CC(=O)Nc1cc(Nc2ncc(Cl)c(-c3cn(C)c4ccccc34)n2)ccc1N(C)CCN(C)C]

To a solution of 24b (264 mg, 1.0 mmol) and 82a (334 mg, 1.2 mmol) in DMA (5 mL) were added Cs₂CO₃ (978 mg, 3.0 mmol), Pd₂(dba)₃ (92 mg, 0.1 mmol) and Xant-phos (116 mg, 0.2 mmol). The mixture was stirred at 120° C. under Ar overnight. After cooling to room temperature, the mixture was diluted with ethyl acetate (500 mL). The organic phase was washed with H₂O (250 mL) and NaCl (250 mL*2), dried with Na₂SO₄. After concentration, the residue was purified by Pre-HPLC (MeCN/0.05% formic acid in water from 0 to 100%) to give 395 as a white solid (62.71 mg, yield 12.8%). MS-ESI (M+H)⁺: 492.3. ¹H NMR (400 MHz, DMSO-d₆) δ 10.01 (s, 1H), 9.54 (s, 1H), 8.65 (d, J=7.6 Hz, 1H), 8.58 (s, 1H), 8.44 (d, J=7.6 Hz, 1H), 8.40 (s, 1H), 7.55-7.48 (m, 2H), 7.30-7.28 (m, 1H), 7.26 (t, J=7.6 Hz, 1H), 7.22 (d, J=7.6 Hz, 1H), 7.15 (t, J=7.6 Hz, 1H), 3.92 (s, 3H), 2.82 (t, J=7.6 Hz, 7.6 Hz, 2H), 2.67 (s, 3H), 2.29 (t, J=7.6 Hz, 7.6 Hz, 2H), 2.23 (s, 6H), 2.06 (s, 3H).

### Procedure for the Synthesis of 396

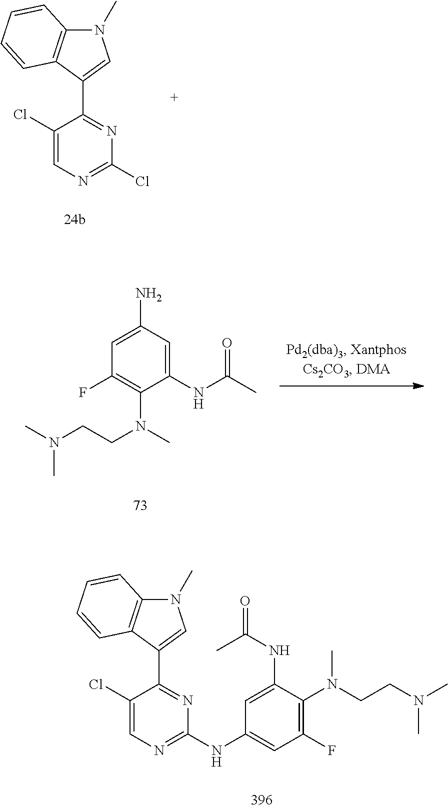 [SMILES: Cn1cc(-c2nc(Cl)ncc2Cl)c2ccccc21 ; CC(=O)Nc1cc(N)cc(F)c1N(C)CCN(C)C ; CC(=O)Nc1cc(Nc2ncc(Cl)c(-c3cn(C)c4ccccc34)n2)cc(F)c1N(C)CCN(C)C]

### N-(5-((5-chloro-4-(1-methyl-1H-indol-3-yl)pyrimidin-2-yl)amino)-2-((2-(dimethylamino)ethyl)(methyl)amino)-3-fluorophenyl)acetamide (396)

To a solution of 24b (414 mg, 1.49 mmol) and 73 (350 mg, 1.24 mmol) in DMA (10 mL) were added Cs₂CO₃ (1.21 g, 3.72 mmol), Pd₂(dba)₃ (114 mg, 0.12 mmol) and Xant-phos (144 mg, 0.24 mmol). The mixture was stirred at 130° C. under Ar for overnight. After cooling to room temperature, the mixture was diluted with dichloromethane (500 mL). The organic phase was washed with H₂O (250 mL) and brine (250 mL*2), dried with Na₂SO₄. After concentration, the residue was purified by Pre-HPLC (MeCN/0.05% NH3·H2O in water from 0 to 100%) to give 396 as a yellow solid (45 mg, yield 7.2%). MS-ESI (M+H)⁺: 510.2. ¹H NMR (400 MHz, DMSO-d6) δ 10.38 (s, 1H), 9.83 (s, 1H), 8.70 (d, J=7.6 Hz, 1H), 8.59 (s, 1H), 8.47 (s, 1H), 8.25 (s, 1H), 7.65-7.61 (m, 1H), 7.56 (d, J=7.6 Hz, 1H), 7.29 (t, J=7.6 Hz, 7.6 Hz, 1H), 7.19 (t, J=7.6 Hz, 7.6 Hz, 1H), 3.93 (s, 3H), 2.99 (m, 2H), 2.75 (s, 3H), 2.22 (s, 8H), 2.07 (s, 3H).

### Procedure for the Synthesis of 397

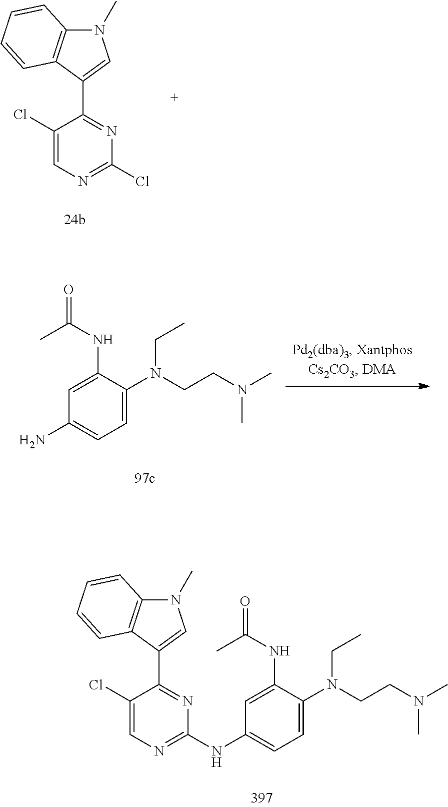 [SMILES: Cn1cc(-c2nc(Cl)ncc2Cl)c2ccccc21 ; CCN(CCN(C)C)c1ccc(N)cc1NC(C)=O ; CCN(CCN(C)C)c1ccc(Nc2ncc(Cl)c(-c3cn(C)c4ccccc34)n2)cc1NC(C)=O]

### N-(5-((5-chloro-4-(1-methyl-1H-indol-3-yl)pyrimidin-2-yl)amino)-2-((2-(dimethylamino)ethyl)(ethyl)amino)phenyl)acetamide (397)

To a solution of 97c (640 mg, 2.42 mmol) and 24b (670 mg, 2.42 mmol) in DMA (10 mL) were added Cs₂CO₃ (2.36 g, 7.26 mmol), Pd₂(dba)₃ (220 mg, 0.24 mmol) and Xant-phos (208 mg, 0.36 mmol). The mixture was stirred at 120° C. under Ar overnight. After cooling to room temperature, the mixture was diluted with dichloromethane (500 mL). The organic phase was washed with H₂O (250 mL) and brine (250 mL*2), dried with Na₂SO₄. After concentration, the residue was purified by Pre-HPLC (MeCN/0.05% NH3·H2O in water from 0 to 100%) to give 397 as a yellow solid (172.57 mg, yield 14.1%). MS-ESI (M+H)⁺: 506.6. ¹H NMR (400 MHz, DMSO-d₆) δ 10.12 (s, 1H), 9.58 (s, 1H), 8.66 (d, J=7.6 Hz, 1H), 8.59 (s, 1H), 8.45-8.42 (m, 2H), 7.55-7.52 (m, 2H), 7.31-7.22 (m, 2H), 7.14 (t, J=7.6 Hz, 1H), 3.93 (s, 3H), 2.99-2.90 (m, 4H), 2.21-2.17 (m, 8H), 2.05 (s, 3H), 0.87 (t, J=7.6 Hz, 3H).

### Procedure for the Synthesis of 398

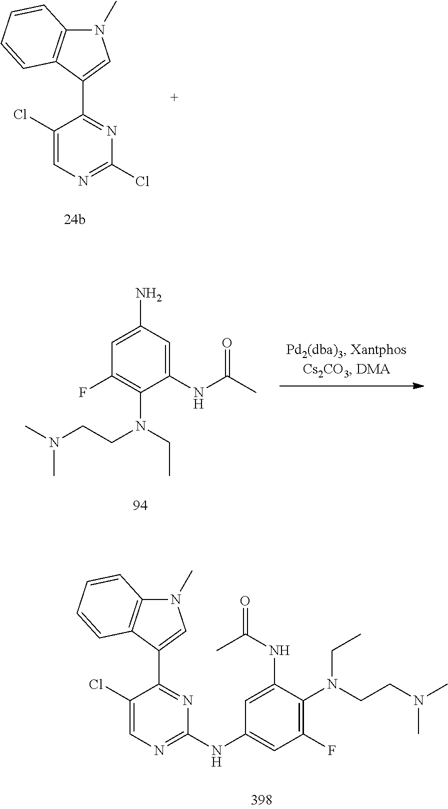 [SMILES: Cn1cc(-c2nc(Cl)ncc2Cl)c2ccccc21 ; CCN(CCN(C)C)c1c(F)cc(N)cc1NC(C)=O ; CCN(CCN(C)C)c1c(F)cc(Nc2ncc(Cl)c(-c3cn(C)c4ccccc34)n2)cc1NC(C)=O]

### N-(5-((5-chloro-4-(1-methyl-1H-indol-3-yl)pyrimidin-2-yl)amino)-2-((2-(dimethylamino)ethyl)(ethyl)amino)-3-fluorophenyl)acetamide (398)

To a solution of 94 (282 mg, 1.0 mmol) and 24b (278 mg, 1.0 mmol) in DMA (5 mL) were added Cs₂CO₃ (0.98 g, 3.0 mmol), Pd₂(dba)₃ (92 mg, 0.1 mmol) and Xant-phos (116 mg, 0.0.2 mmol). The mixture was stirred at 120° C. under Ar overnight. After cooling to room temperature, the mixture was diluted with dichloromethane (200 mL). The organic phase was washed with H₂O (200 mL) and brine (200 mL), dried with Na₂SO₄. After concentration, the residue was purified by Pre-HPLC (MeCN/0.05% NH3·H2O in water from 0 to 100%) to give 398 as a yellow solid (102.95 mg, yield 19.6%). MS-ESI (M+H)⁺: 524.2. ¹H NMR (400 MHz, DMSO-d6) δ 10.42 (s, 1H), 9.83 (s, 1H), 8.70 (d, J=7.6 Hz, 1H), 8.60 (s, 1H), 8.48 (s, 1H), 8.22 (s, 1H), 7.65-7.61 (m, 1H), 7.56 (d, J=7.6 Hz, 1H), 7.30 (t, J=7.6 Hz, 1H), 7.18 (t, J=7.6 Hz, 1H), 3.93 (s, 3H), 3.07-3.02 (m, 4H), 2.21 (s, 8H), 2.06 (s, 3H), 0.88 (t, J=7.6 Hz, 3H).

### Procedure for the Syntheses of 400a and 400b

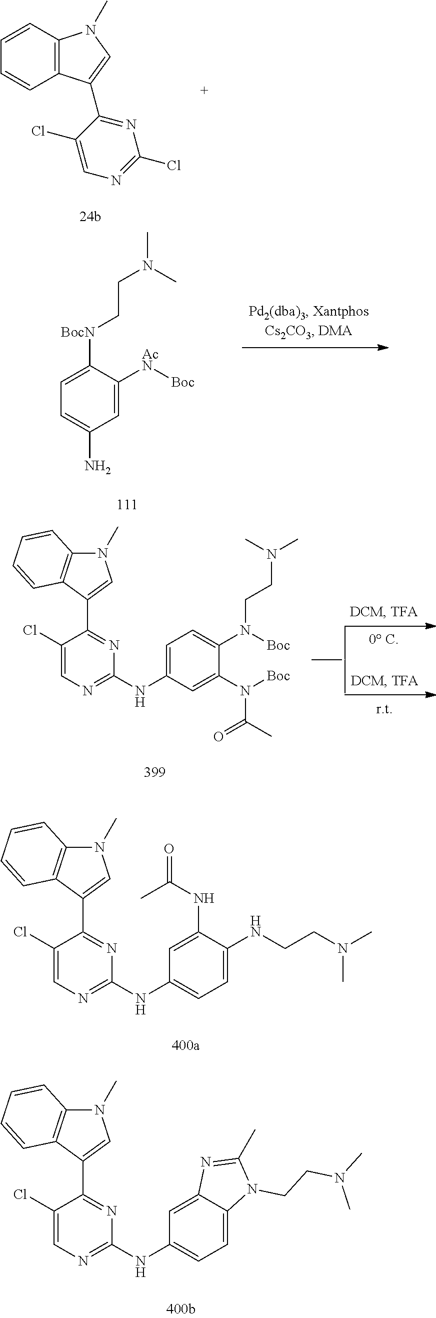

### tert-butylacetyl(2-((tert-butoxycarbonyl)(2-(dimethylamino)ethyl)amino)-5-((5-chloro-4-(1-methyl-1H-indol-3-yl)pyrimidin-2-yl)amino)phenyl)carbamate (399)

To a solution of 111 (702 mg, 1.61 mmol) and 24b (446 mg, 1.61 mmol) in DMA (5 mL) were added Cs₂CO₃ (1.04 g, 3.2 mmol), Pd₂(dba)₃ (147 mg, 0.16 mmol) and Xant-phos (93 mg, 0.16 mmol). The mixture was stirred at 120° C. under microwave for 2 h. After cooling to room temperature, the mixture was diluted with dichloromethane (100 mL). The organic phase was washed with H₂O (100 mL) and brine (100 mL), dried over Na₂SO₄. After concentration, the residue was purified by pre-TLC (dichloromethane/methanol=12/1) to give 399 as a yellow solid (220 mg, yield 20%). MS-ESI (M+H)⁺: 678.3. ¹H NMR (400 MHz, DMSO-d₆) δ 9.99 (s, 1H), 8.62 (d, J=7.6 Hz, 1H), 8.57 (s, 1H), 8.52 (s, 1H), 7.92 (s, 1H), 7.82-7.79 (m, 1H), 7.58 (d, J=7.6 Hz, 1H), 7.31 (t, J=7.6 Hz, 1H), 7.21 (t, J=7.6 Hz, 1H), 3.94 (s, 3H), 2.95 (t, J=,7.6 Hz, 2H), 2.26 (s, 6H), 2.03-1.97 (m, 2H), 1.75 (s, 3H), 1.39 (s, 9H), 1.23 (s, 9H).

### N-(5-((5-chloro-4-(1-methyl-1H-indol-3-yl)pyrimidin-2-yl)amino)-2-((2-(dimethylamino)ethyl)amino)phenyl)acetamide (400a)

To a solution of 399 (200 mg, 0.3 mmol) in dichloromethane (100 mL) was added TFA (50 mL) at 0° C. The mixture was stirred at this temperature for 2 h. After diluting with dichloromethane (100 mL), the mixture was neutralized to pH=7 with saturated Na2CO3. The organic phase was washed with brine (100 mL), dried with Na₂SO₄. After concentration, the residue was purified by pre-TLC (dichloromethane/methanol=15/1) to give 400a as a yellow solid (34.34 mg, yield 23%). MS-ESI (M+H)⁺: 478.3. ¹H NMR (400 MHz, DMSO-d₆) δ 9.49 (s, 1H), 8.79 (d, J=7.6 Hz, 1H), 8.57 (s, 1H), 8.43 (s, 1H), 7.55 (d, J=7.6 Hz, 1H), 7.30 (t, J=7.6 Hz, 1H), 7.19-7.16 (m, 2H), 7.00 (t, J=7.6 Hz, 1H), 6.90 (d, J=7.6 Hz, 1H), 3.93 (br, 5H), 2.27 (br, 8H), 1.74 (s, 3H).

### N-(5-chloro-4-(1-methyl-1H-indol-3-yl)pyrimidin-2-yl)-1-(2-(dimethylamino)ethyl)-2-methyl-1H-benzo[d]imidazol-5-amine (400b)

To a solution of 399 (120 mg, 0.18 mmol) in dichloromethane (15 mL) was added TFA (5 mL) and the mixture was stirred at r t for 2 h. After diluting with dichloromethane (100 mL), the mixture was neutralized to pH=7 with saturated Na2CO3. The organic phase was washed with brine (100 mL), dried with Na₂SO₄. After concentration, the residue was purified by Pre-HPLC (MeCN/0.05% NH₃—H₂O in water from 0 to 100%) to give 400b as a yellow solid (37.29 mg, yield 46%). MS-ESI (M+H)⁺: 460.3. ¹H NMR (400 MHz, DMSO-d₆) δ 9.47 (s, 1H), 8.60 (d, J=7.6 Hz, 1H), 8.55 (s, 1H), 8.41 (s, 1H), 7.89 (s, 1H), 7.52 (d, J=7.6 Hz, 1H), 7.49-7.47 (m, 1H), 7.40 (d, J=7.6 Hz, 1H), 7.27 (t, J=7.6 Hz, 1H), 7.09 (t, J=7.6 Hz, 1H), 4.23 (t, J=7.6 Hz, 2H), 3.93 (s, 3H), 2.58 (t, J=7.6 Hz, 2H), 2.53 (s, 3H), 2.22 (s, 6H).

### Procedure for the Synthesis of 401

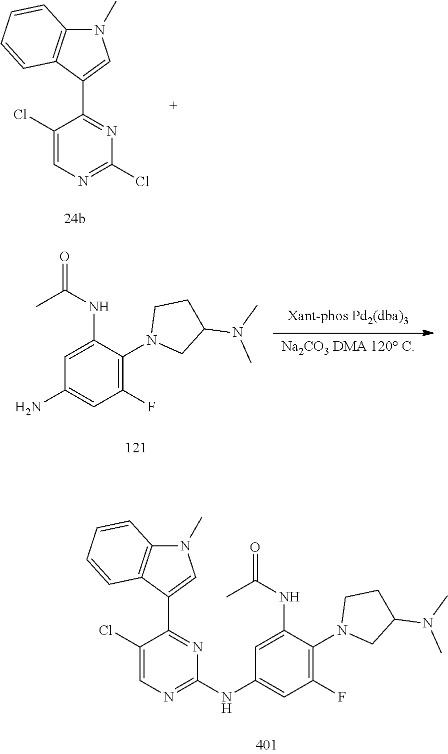 [SMILES: Cn1cc(-c2nc(Cl)ncc2Cl)c2ccccc21 ; CC(=O)Nc1cc(N)cc(F)c1N1CCC(N(C)C)C1 ; CC(=O)Nc1cc(Nc2ncc(Cl)c(-c3cn(C)c4ccccc34)n2)cc(F)c1N1CCC(N(C)C)C1]

### N-(5-((5-chloro-4-(1-methyl-1H-indol-3-yl)pyrimidin-2-yl)amino)-2-(3-(dimethylamino)pyrrolidin-1-yl)-3-fluorophenyl)acetamide (401)

To a mixture of 121 (200 mg, 0.72 mmol), 24b (220 mg, 0.8 mmol) in DMA (15 mL) were added Xant-phos (24 mg, 0.04 mmol), Pd₂(dba)₃ (36 mg, 0.04 mmol) and Na₂CO₃ (200 mg, 1.8 mmol). The mixture was stirred at 120° C. under Ar overnight. After cooling to room temperature, the mixture was diluted with ethyl acetate (200 mL). The organic phase was washed with H₂O (250 mL) and brine (250 mL*2), dried with Na₂SO₄. After concentration, the residue was purified by pre-HPLC to give the title product 401 as a yellow solid (65 mg, yield 16%) m/z (MS-ESI) (M+H)⁺: 522.3. ¹H NMR (400 MHz, CD₃OD) δ 8.61 (d, J=8.0 Hz, 1H), 8.40 (s, 1H), 8.33 (s, 1H), 8.23 (s, 1H), 7.60 (d, J=14.4 Hz, 1H), 7.54 (d, J=7.6 Hz, 1H), 7.29 (t, J=8.0 Hz, 1H), 7.17 (t, J=7.6 Hz, 1H), 3.90 (s, 3H), 3.28-3.18 (m, 4H), 3.04-2.97 (m, 1H), 2.32 (s, 6H), 2.22-2.18 (m, 4H), 2.00-1.93 (m, 1H).

### Procedure for the Synthesis of 402

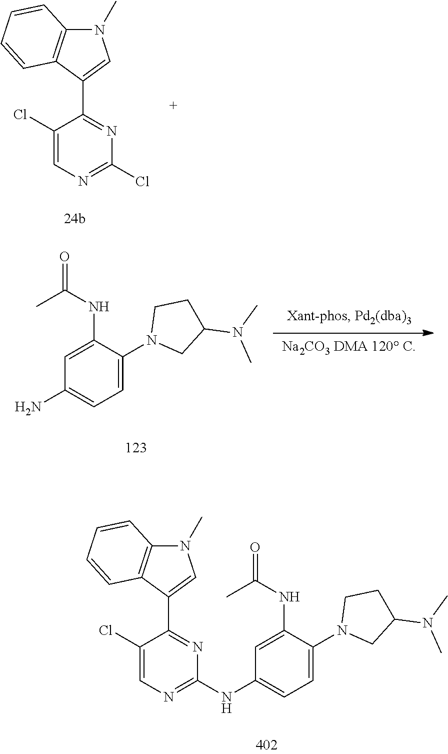 [SMILES: Cn1cc(-c2nc(Cl)ncc2Cl)c2ccccc21 ; CC(=O)Nc1cc(N)ccc1N1CCC(N(C)C)C1 ; CC(=O)Nc1cc(Nc2ncc(Cl)c(-c3cn(C)c4ccccc34)n2)ccc1N1CCC(N(C)C)C1]

### N-(5-((5-chloro-4-(1-methyl-1H-indol-3-yl) pyrimidin-2-yl)amino)-2-(3-(dimethylamino)pyrrolidin-1-yl)phenyl)acetamide (402)

Synthesis of 402 was similar to that of 401. After concentration, the residue was purified by Pre-HPLC (MeCN/0.05% NH₄OH in water from 0 to 100%) to give the title product as a yellow solid (105.7 mg, 0.21 mmol, 19%). ¹H NMR (400 MHz, DMSO-d₆) δ 9.40 (s, 1H), 9.13 (s, 1H), 8.62 (d, J=6.5 Hz, 1H), 8.55 (d, J=7.1 Hz, 1H), 8.38 (s, 1H), 7.77 (s, 1H), 7.54 (d, J=8.2 Hz, 1H), 7.45 (d, J=7.2 Hz, 1H), 7.29 (t, J=7.1 Hz, 1H), 7.19 (t, J=7.4 Hz, 1H), 6.86 (d, J=8.8 Hz, 1H), 3.92 (s, 3H), 3.19-3.04 (m, 3H), 2.80-2.70 (m, 1H), 2.18 (s, 6H), 2.01 (s, 2H), 1.76-1.72 (m, 1H).

### Procedure for the Synthesis of 403

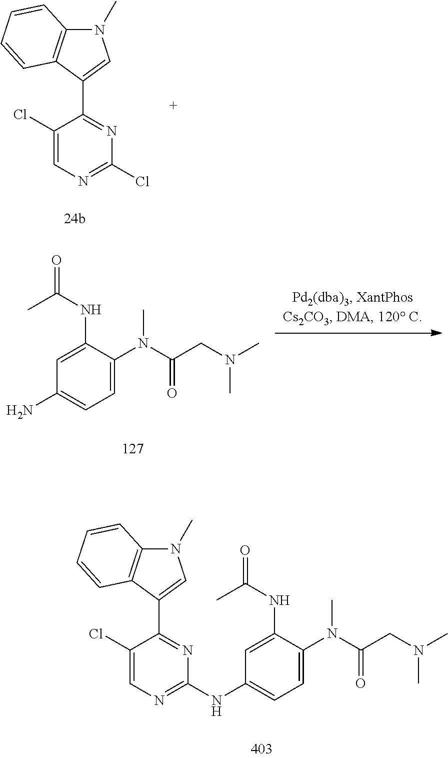 [SMILES: Cn1cc(-c2nc(Cl)ncc2Cl)c2ccccc21 ; CC(=O)Nc1cc(N)ccc1N(C)C(=O)CN(C)C ; CC(=O)Nc1cc(Nc2ncc(Cl)c(-c3cn(C)c4ccccc34)n2)ccc1N(C)C(=O)CN(C)C]

### N-(2-acetamido-4-((5-chloro-4-(1-methyl-1H-indol-3-yl)pyrimidin-2-yl)amino)phenyl)-2-(dimethylamino)-N-methylacetamide (403)

Synthesis of 403 was similar to that of 395. The residue was purified by pre-HPLC (MeCN/0.1% FA in water from 0 to 100%) to afford 403 (36 mg, yield 11%) as a yellow solid. MS-ESI (M+H)⁺: 506.3. ¹H NMR (400 MHz, CD3OD) δ 8.59 (dd, J=11.2, 5.2 Hz, 2H), 8.44 (s, 1H), 8.36 (s, 1H), 7.64 (dd, J=8.6, 2.5 Hz, 1H), 7.47 (d, J=8.2 Hz, 1H), 7.29 (t, J=7.5 Hz, 1H), 7.22 (d, J=8.6 Hz, 1H), 7.16 (t, J=7.6 Hz, 1H), 3.92 (s, 3H), 3.21 (s, 3H), 3.06 (s, 2H), 2.34 (s, 6H), 1.88 (s, 3H).

### Procedure for the Synthesis of 404

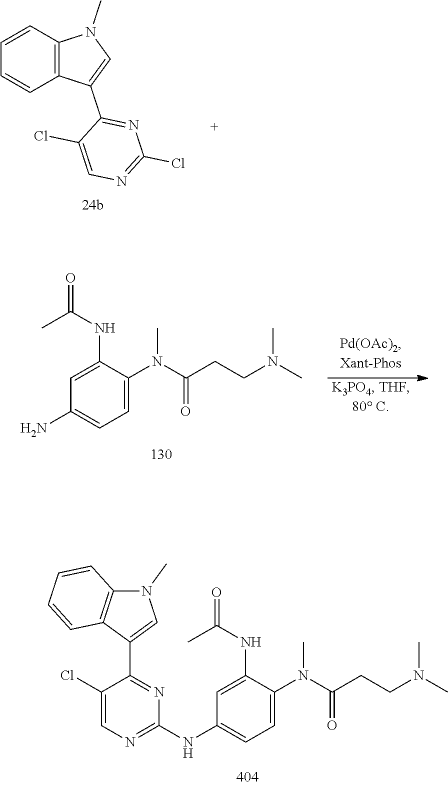 [SMILES: Cn1cc(-c2nc(Cl)ncc2Cl)c2ccccc21 ; CC(=O)Nc1cc(N)ccc1N(C)C(=O)CCN(C)C ; "K3PO4, THF, 80° C." ; CC(=O)Nc1cc(Nc2ncc(Cl)c(-c3cn(C)c4ccccc34)n2)ccc1N(C)C(=O)CCN(C)C]

### N-(2-acetamido-4-((5-chloro-4-(1-methyl-1H-indol-3-yl) pyrimidin-2-yl) amino) phenyl)-3-(dimethylamino)-N-methylpropanamide (404)

To a solution of 24b (250 mg, 0.9 mmol) in 25 mL of THF was added 130 (250 mg, 0.9 mmol), Pd(OAc)₂ (20 mg, 0.09 mmol), Xant-Phos (78 mg, 0.135 mmol) and K₃PO₄ (380 mg, 1.8 mmol). The mixture was stirred at 80° C. under Ar overnight. After cooling to room temperature, the mixture was filtered over Celite and washed with THF to give a solution. The solution was concentrated to give a residue. The residue was purified by Prep-HPLC (MeCN/0.1% formic acid in water from 0 to 100%) to give the title product as a formate, which was neutralized with saturated Na₂CO₃ to afford 404 (110 mg, yield 25.6%) as a yellow solid. MS-ESI (M+H): 520.3. ¹H NMR (400 MHz, CD3OD) δ 8.61 (d, J=8.0 Hz, 1H), 8.41 (s, 1H), 8.36 (s, 1H), 8.33 (d, J=2.2 Hz, 1H), 7.64 (dd, J=8.6, 2.5 Hz, 1H), 7.47 (d, J=8.2 Hz, 1H), 7.32-7.27 (m, 1H), 7.22-7.16 (m, 2H), 3.91 (s, 3H), 3.17 (s, 3H), 2.74 (dt, J=12.5, 7.4 Hz, 1H), 2.51 (dt, J=12.6, 6.5 Hz, 1H), 2.34-2.27 (m, 2H), 2.14 (s, 6H), 2.10 (s, 3H).

### Procedure for the Synthesis of 405

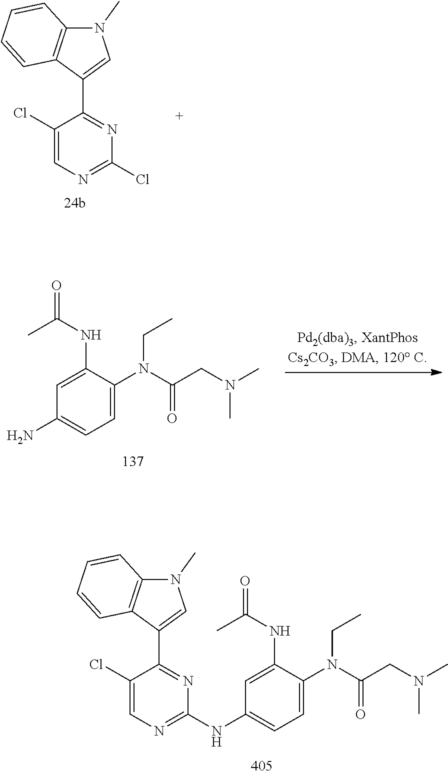 [SMILES: Cn1cc(-c2nc(Cl)ncc2Cl)c2ccccc21 ; CCN(C(=O)CN(C)C)c1ccc(N)cc1NC(C)=O ; CCN(C(=O)CN(C)C)c1ccc(Nc2ncc(Cl)c(-c3cn(C)c4ccccc34)n2)cc1NC(C)=O]

### N-(2-acetamido-4-((5-chloro-4-(1-methyl-1H-indol-3-yl) pyrimidin-2-yl) amino) phenyl)-2-(dimethylamino)-N-ethylacetamide (405)

Synthesis of 405 was similar to that of 395. The residue was purified by Pre-HPLC (MeCN/0.05% NH₄OH in water from 0 to 100%) to afford 405 (33.6 mg, yield 28.2%) as a yellow solid. MS-ESI (M+H)⁺: 520.3. ¹H NMR (400 MHz, CD₃OD) δ 8.50 (s, 1H), 8.31 (s, 1H), 8.21 (d, J=8.2 Hz, 1H), 8.17 (d, J=2.2 Hz, 1H), 7.66 (dd, J=8.6, 2.3 Hz, 1H), 7.44 (d, J=8.2 Hz, 1H), 7.25 (dd, J=11.9, 7.1 Hz, 2H), 6.98 (t, J=7.6 Hz, 1H), 3.96 (dd, J=13.6, 7.0 Hz, 1H), 3.90 (s, 3H), 3.41 (dd, J=13.7, 7.1 Hz, 1H), 2.96 (d, J=15.2 Hz, 1H), 2.85 (d, J=15.2 Hz, 1H), 2.07 (s, 3H), 2.01 (s, 6H), 1.11 (t, J=7.1 Hz, 3H).

### Procedure for the Synthesis of 406

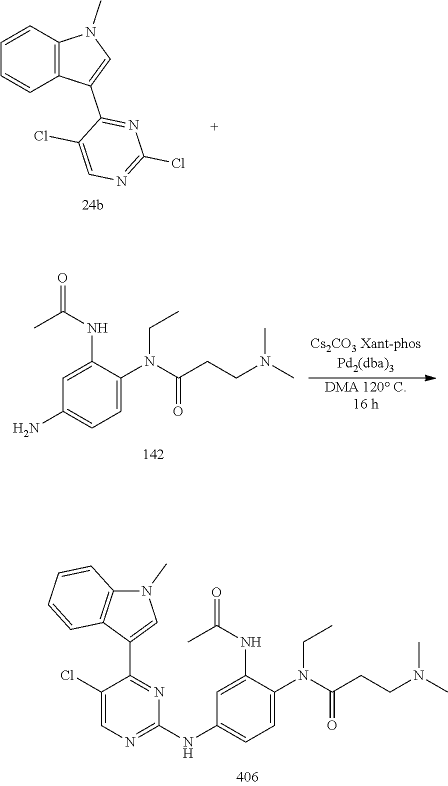 [SMILES: Cn1cc(-c2nc(Cl)ncc2Cl)c2ccccc21 ; CCN(C(=O)CCN(C)C)c1ccc(N)cc1NC(C)=O ; "DMA 120° C.16 h" ; CCN(C(=O)CCN(C)C)c1ccc(Nc2ncc(Cl)c(-c3cn(C)c4ccccc34)n2)cc1NC(C)=O]

### N-(2-acetamido-4-((5-chloro-4-(1-methyl-1H-indol-3-yl)pyrimidin-2-yl)amino)phenyl)-3-(dimethylamino)-N-ethylpropanamid (406)

A mixture of 142 (584.0 mg, 2.0 mmol), Xant-phos (175 mg, 0.3 mmol), Cs₂CO₃ (1.2 g, 4.0 mmol), 24b (556 mg, 2.0 mmol) and Pd₂(dba)₃ (185 mg, 0.1 mmol) in DMA (50 mL) was stirred at 120° C. under Ar overnight. After cooling to room temperature, the mixture was filtered and the filtrate was purified by pre-HPLC (MeCN/0.05% NH₃ in water from 0 to 100%) to give the title product 406 as a yellow solid (122.0 mg, yield 11.5%). MS-ESI (M+H)⁺: 533.2. ¹H NMR (400 MHz, CDCl₃) δ 9.80 (s, 1H), 8.07 (s, 1H), 7.93 (d, J=6.3 Hz, 1H), 7.67 (d, J=11.6 Hz, 1H), 7.43 (d, J=8.6 Hz, 1H), 7.28-7.20 (m, 1H), 6.94 (d, J=8.0 Hz, 1H), 6.64 (d, J=6.2 Hz, 1H), 6.55 (d, J=8.7 Hz, 1H), 6.17 (d, J=8.1 Hz, 1H), 6.06 (s, 1H), 4.06-3.81 (m, 4H), 3.49-3.46 (m, 1H), 3.27 (s, 4H), 2.52-2.50 (m, 2H), 2.23 (s, 2H), 2.12-2.10 (m, 2H), 2.05-2.03 (m, 7H), 1.05-1.00 (m, 3H).

### Procedure for the Synthesis of 407

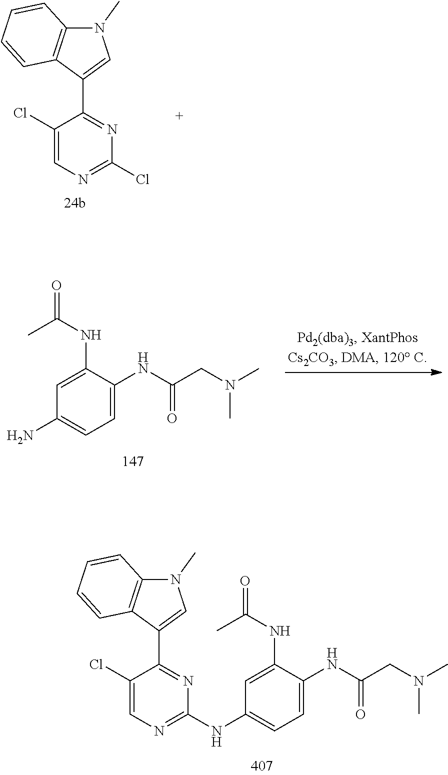 [SMILES: Cn1cc(-c2nc(Cl)ncc2Cl)c2ccccc21 ; CC(=O)Nc1cc(N)ccc1NC(=O)CN(C)C ; CC(=O)Nc1cc(Nc2ncc(Cl)c(-c3cn(C)c4ccccc34)n2)ccc1NC(=O)CN(C)C]

### N-(2-acetamido-4-((5-chloro-4-(1-methyl-1H-indol-3-yl)pyrimidin-2-yl)amino)phenyl)-2-(dimethylamino)acetamide (407)

To the mixture of 147 (400 mg, 1.60 mmol) and 3-(2,5-dichloropyrimidin-4-yl)-1-methyl-1H-indole (488 mg, 1.76 mmol) 24b in DMA (12 mL) were added Pd₂(dba)₃ (146 mg, 0.16 mmol), XantPhos (184 mg, 0.32 mmol) and Cs₂CO₃ (1.042 g, 3.20 mmol) under Ar. The mixture was stirred at 120° C. under Ar for 16 h. The mixture was purified by Prep-HPLC (NH₃—H₂O) to give 407 (56.24 mg, yield 7.15%) as grey solid. MS-ESI (M+H⁺): 492.1. ¹H NMR (400 MHz, DMSO) δ 9.72 (d, J=13.5 Hz, 2H), 9.31 (s, 1H), 8.62 (d, J=8.0 Hz, 1H), 8.56 (s, 1H), 8.46 (s, 1H), 7.81 (s, 1H), 7.63 (d, J=8.8 Hz, 1H), 7.54 (t, J=10.0 Hz, 2H), 7.30 (t, J=7.6 Hz, 1H), 7.18 (t, J=7.4 Hz, 1H), 3.93 (s, 3H), 3.03 (s, 2H), 2.29 (s, 6H), 2.02 (s, 3H).

### Procedure for the Synthesis of 408

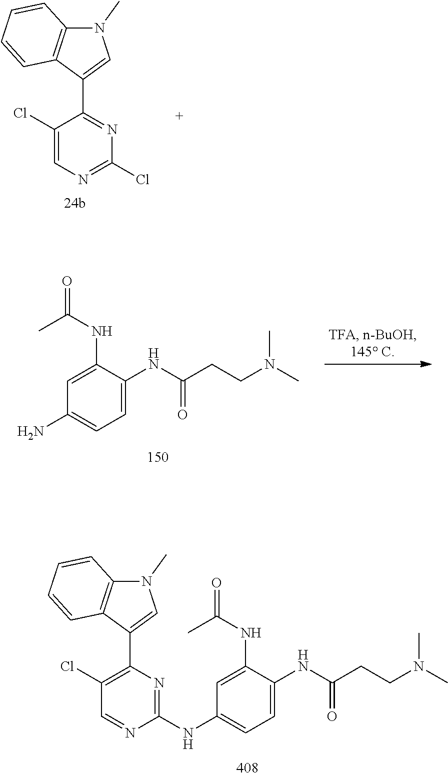 [SMILES: Cn1cc(-c2nc(Cl)ncc2Cl)c2ccccc21 ; CC(=O)Nc1cc(N)ccc1NC(=O)CCN(C)C ; CC(=O)Nc1cc(Nc2ncc(Cl)c(-c3cn(C)c4ccccc34)n2)ccc1NC(=O)CCN(C)C]

### N-(2-acetamido-4-((5-chloro-4-(1-methyl-1H-indol-3-yl)pyrimidin-2-yl)amino)phenyl)-3-(dimethylamino)propanamide (408)

To the mixture of 147 (200 mg, 0.76 mmol) and 3-(2,5-dichloropyrimidin-4-yl)-1-methyl-1H-indole 24b (221 mg, 0.79 mmol) in n-BuOH (6 mL) was added TFA (0.11 mL, 1.51 mmol). The mixture was stirred at 145° C. under Ar for 16 h. The mixture was concentrated and the residue was purified by Prep-HPLC (FA) to give the product. The product was basified by aqueous Na₂CO₃ to give 408 (39.92 mg, 0.078 mmol, 10.43% yield) as white solid. MS-ESI (M+H⁺): 506.2. ¹H NMR (400 MHz, DMSO-d₆) δ 9.66 (s, 2H), 9.23 (s, 1H), 8.64 (d, J=7.9 Hz, 1H), 8.57 (s, 1H), 8.45 (s, 1H), 7.96 (s, 1H), 7.55 (d, J=8.2 Hz, 2H), 7.49 (d, J=8.8 Hz, 1H), 7.30 (t, J=7.4 Hz, 1H), 7.20 (t, J=7.5 Hz, 1H), 3.93 (s, 3H), 2.56 (t, J=6.5 Hz, 2H), 2.45 (t, J=6.6 Hz, 2H), 2.20 (s, 6H), 2.02 (s, 3H).

### Procedure for the Synthesis of S-409

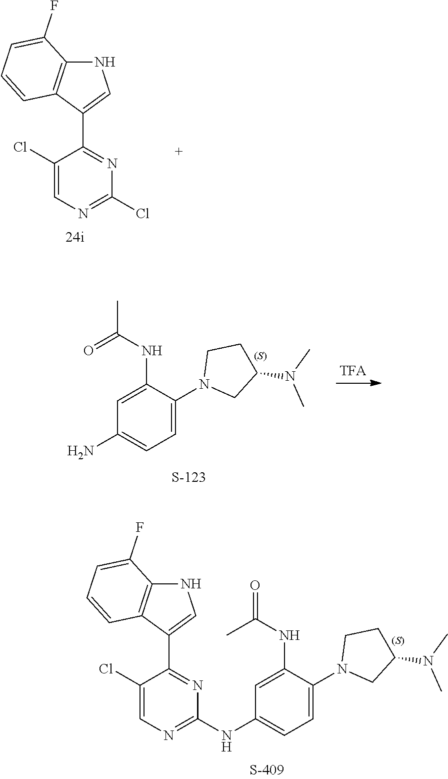

### (S)—N-(5-((5-chloro-4-(7-fluoro-1H-indol-3-yl)pyrimidin-2-yl)amino)-2-(3-(dimethylamino)pyrrolidin-1-yl)phenyl)acetamide (S-409)

The compound 24i (0.12 g, 0.42 mmol), S-123 (0.10 g, 0.38 mmol), trifluoroacetic acid (0.10 g, 0.88 mmol) and 1,4-dioxane (5 ml) were added to the flask. The reaction mixture was stirred for 48 h at refluxing temperature. The reaction mixture was concentrated and the residue was dissolved with ethyl acetate (100 ml) and washed with water (50 ml*3). The organic layer was concentrated and the residue was purified by flash column chromatography (dichloromethane/methanol=20/1) to give the compound S-409 (22 mg, yield 11.41%). MS-ESI (M+H)⁺:508.3.

### Procedure for the Syntheses of 411 and 412

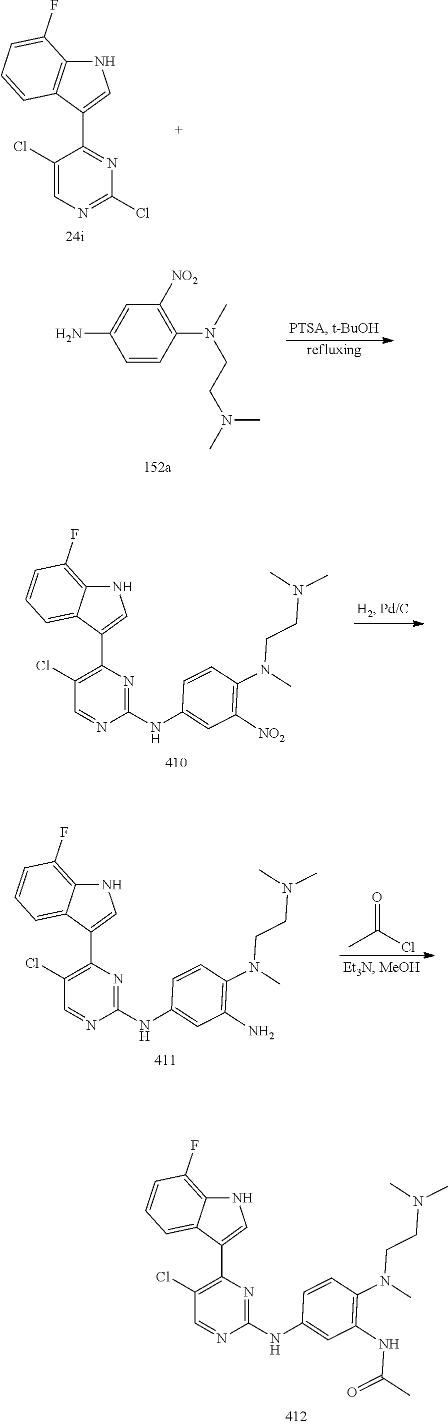

### N⁴-(5-chloro-4-(7-fluoro-1H-indol-3-yl)pyrimidin-2-yl)-N-(2-(dimethylamino)ethyl)-N¹-methyl-2-nitrobenzene-1,4-diamine (410)

The compound 24i (0.35 g, 1.24 mmol), N¹-(2-(dimethylamino) ethyl)-N¹-methyl-2-nitrobenzene-1,4-diamine 152a (0.30 g, 1.26 mmol), PTSA (0.51 g, 3.69 mmol) and t-BuOH (10 ml) were added to the flask. The resulting mixture was stirred over night at 85° C. The reaction mixture was cooled down and filtered. The filtrate was concentrated. The residue was dissolved with ethyl acetate (200 ml) and washed with water (100 ml*3). The organic layer was concentrated and the residue was purified by silica gel column (dichloromethane/methanol=20/1) to give the title compound 410 (0.23 g, yield 38.31%). MS-ESI (M+H)⁺:484.3.

### N⁴-(5-chloro-4-(7-fluoro-1H-indol-3-yl)pyrimidin-2-yl)-N¹-(2-(dimethylamino)ethyl)-N¹-methylbenzene-1,2,4-triamine (411)

The compound 410 (0.23 g, 0.47 mmol) was mixed with Pd/C (5%, 0.5 g, 0.23 mmol) in methanol (20 ml). The resulting mixture was stirred at room temperature overnight under hydrogen atmosphere. The reaction mixture was filtered and concentrated. The residue was purified by silica gel column (dichloromethane/methanol=15/1) to give the title compound 411 (0.24 g, yield 100%). MS-ESI (M+H)⁺: 454.3. ¹H NMR (500 MHz, DMSO-d₆) δ 12.54 (s, 1H), 9.29 (s, 1H), 8.47 (s, 1H), 8.41 (s, 2H), 7.10-7.02 (m, 2H), 7.00 (d, J=1.8 Hz, 1H), 6.92-6.86 (m, 2H), 3.17 (s, 2H), 2.96 (s, 2H), 2.56 (s, 3H), 2.51 (d, J=5.3 Hz, 8H), 2.39 (s, 6H).

### N-(5-((5-chloro-4-(7-fluoro-1H-indol-3-yl)pyrimidin-2-yl)amino)-2-((2-(dimethylamino)ethyl)(methyl)amino)phenyl)acetamide (412)

Triethylamine (0.1 g, 0.99 mmol) was added to the solution of compound 411 (100 mg, 0.22 mmol) in methanol (10 ml). And then the mixture was cooled by ice-salt bath. Acetyl chloride (0.06 g, 0.76 mmol) was added dropwise to the mixture. After stirring for 0.5 h, the reaction mixture was concentrated. The residue was dissolved in ethyl acetate (50 ml) and washed by NaHCO₃ aqueous. The organic layer was separated and concentrated in vacuo. The residue was purified by silica gel column (dichloromethane/methanol=20/1) to give the title compound 412 (54 mg, yield 49.58%). MS-ESI (M+H)⁺: 496.19. ¹H NMR (500 MHz, DMSO-d₆) δ 12.60 (s, 1H), 9.62 (s, 1H), 8.51 (s, 1H), 8.45 (s, 1H), 8.37 (s, 1H), 7.52 (dd, J=8.6, 2.4 Hz, 1H), 7.20 (d, J=8.7 Hz, 1H), 7.11-7.04 (m, 2H), 3.39 (s, 7H), 3.17 (s, 1H), 2.99 (s, 3H), 2.61 (s, 3H), 2.51 (d, J=3.5 Hz, 4H), 2.45 (s, 5H), 2.15 (s, 3H).

### Procedure for the Synthesis of R-414

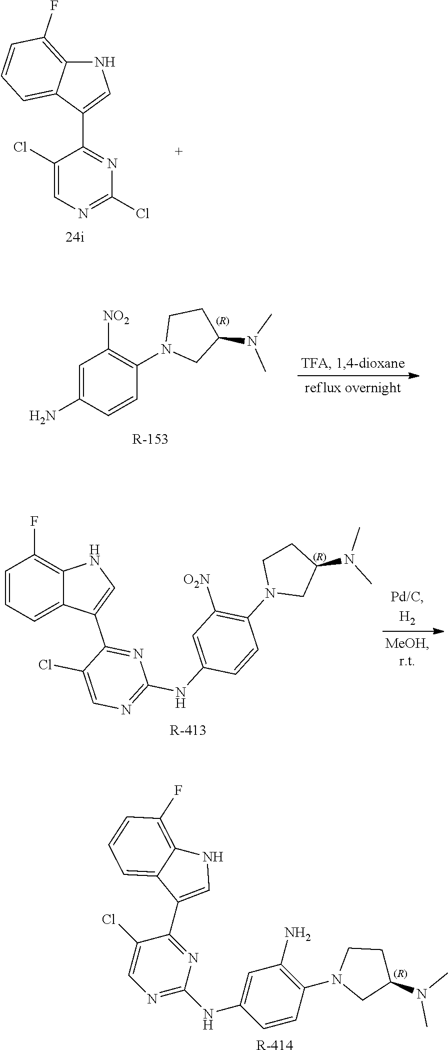

### (R)-5-chloro-N-(4-(3-(dimethylamino)pyrrolidin-1-yl)-3-nitrophenyl)-4-(7-fluoro-1-methyl-1H-indol-3-yl)pyrimidin-2-amine (R-413)

To the mixture of 24i (0.310 g, 1.0 mmol) and R-153 (0.250 g, 1.0 mmol) in 1,4-dioxane (20 mL) was added TFA (0.25 mL), then the mixture was refluxed overnight. After cooling to room temperature and concentration, the residue was purified by flash column chromatography (dichloromethane/methanol=30/1-20/1) to give crude, followed by recrystallization with ethanol to give R-413 as brownish red solid (100 mg, yield 19.2%). MS-ESI (M+H)⁺:522.4.

### (R)—N¹-(5-chloro-4-(7-methoxy-1-methyl-1H-indol-3-yl)pyrimidin-2-yl)-4-(3-(dimethylamino)pyrrolidin-1-yl)benzene-1,3-diamine (R-414)

To the mixture of R-413 (0.060 g, 0.196 mmol) in methanol (30 mL) were added Pd/C (5% w/w, 20 mg), then the mixture was stirred under H₂ at room temperature for 3 h. The reaction mixture was filtered through celite and the filtrate was concentrated under reduced pressure to give a residue. The residue was purified by flash column chromatography (dichloromethane/methanol=30/1-20/1) and preparative thin layer chromatography to give R-414 (8 mg, 9% yield) as a brown solid. MS-ESI (M+H)⁺: 466.3.

### Procedure for the Synthesis of S-414

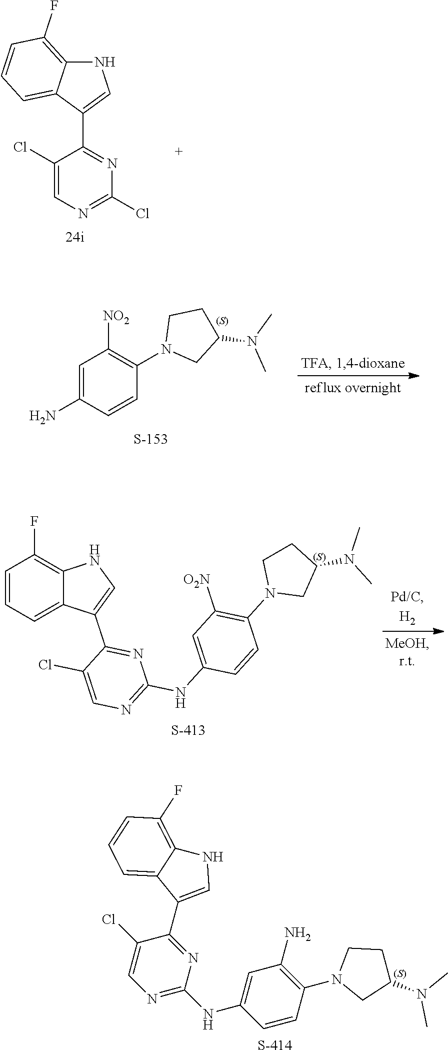

### (S)-5-chloro-N-(4-(3-(dimethylamino)pyrrolidin-1-yl)-3-nitrophenyl)-4-(7-fluoro-1-methyl-1H-indol-3-yl)pyrimidin-2-amine (S-413)

Synthesis of S-413 was similar to that of R-413. The residue was purified by preparative thin layer chromatography (dichloromethane/methanol=15/1) to give the title product S-413 (80 mg, yield 16.1%) as a gray solid. MS-ESI (M+H)⁺:496.4.

### (S)—N¹-(5-chloro-4-(7-fluoro-1H-indol-3-yl)pyrimidin-2-yl)-4-(3-(dimethylamino)pyrrolidin-1-yl)benzene-1,3-diamine (S-414)

S-413 (80 mg, 0.15 mmol) was mixed with Pd/C (0.02 g) in methanol (10 ml). The resulting mixture was stirred at room temperature overnight under hydrogen atmosphere. The reaction mixture was filtered and concentrated. The residue was purified by flash column chromatography (dichloromethane/methanol=15/1) to give the compound S-414 (35.9 mg, yield 51.46%) MS-ESI (M+H)+: 466.2.

### Procedure for the Synthesis of R-415

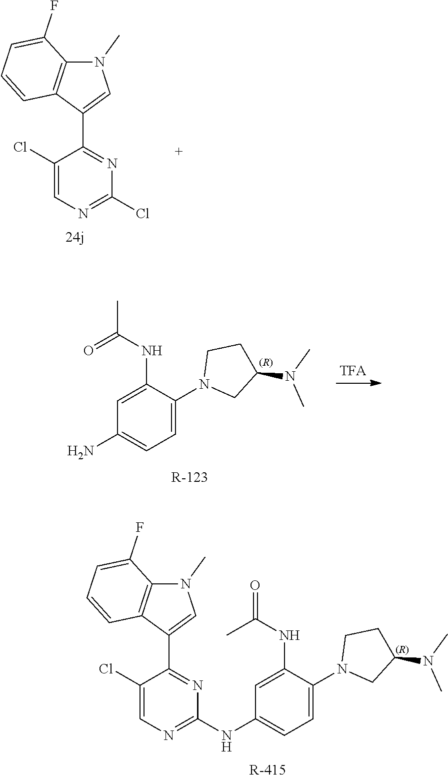

### (R)—N-(5-((5-chloro-4-(7-fluoro-1-methyl-1H-indol-3-yl)pyrimidin-2-yl)amino)-2-(3-(dimethylamino)pyrrolidin-1-yl)phenyl)acetamide (R-415)

24j (0.15 g, 0.44 mmol), S-123 (0.14 g, 0.53 mmol), trifluoroacetic acid (0.30 g, 2.60 mmol) and 1,4-dioxane (30 ml) was added to the flask. The reaction mixture was stirred over night at refluxing temperature. The reaction mixture was concentrated and the residue was dissolved with ethyl acetate (100 ml) and washed with water (50 ml*3). The organic layer was concentrated and the residue was purified by flash column chromatography (dichloromethane/methanol=15:1) to give the compound R-415 (17 mg, yield 7.4%). MS-ESI (M+H)⁺:522.5.

### Procedure for the Synthesis of S-415

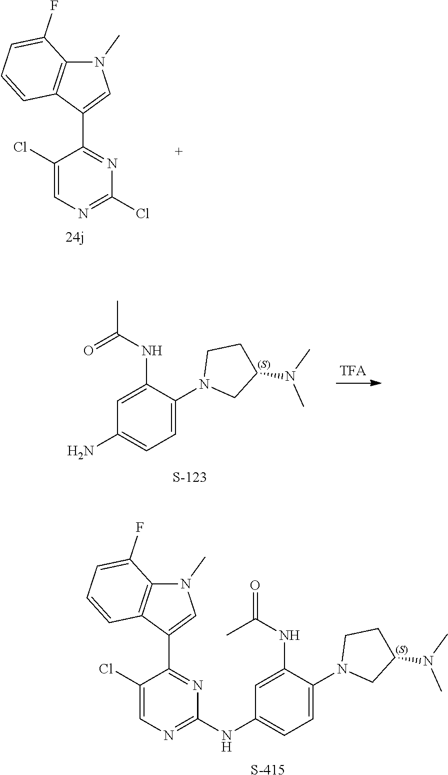

### (S)—N-(5-((5-chloro-4-(7-fluoro-1-methyl-1H-indol-3-yl)pyrimidin-2-yl)amino)-2-(3-(dimethylamino)pyrrolidin-1-yl)phenyl)acetamide (S-415)

24j (0.13 g, 0.43 mmol), S-123 (0.10 g, 0.38 mmol), trifluoroacetic acid (0.10 g, 0.88 mmol) and 1,4-dioxane (5 ml) was added to the flask. The reaction mixture was stirred for 48 h at refluxing temperature. The reaction mixture was concentrated and the residue was dissolved with ethyl acetate (100 ml) and washed with water (50 ml*3). The organic layer was concentrated and the residue was purified by flash column chromatography (dichloromethane/methanol=20/1) to give the compound S-415 (15 mg, yield 7.57%). MS-ESI (M+H)⁺:522.4.

### Procedure for the Syntheses of 417 and 418

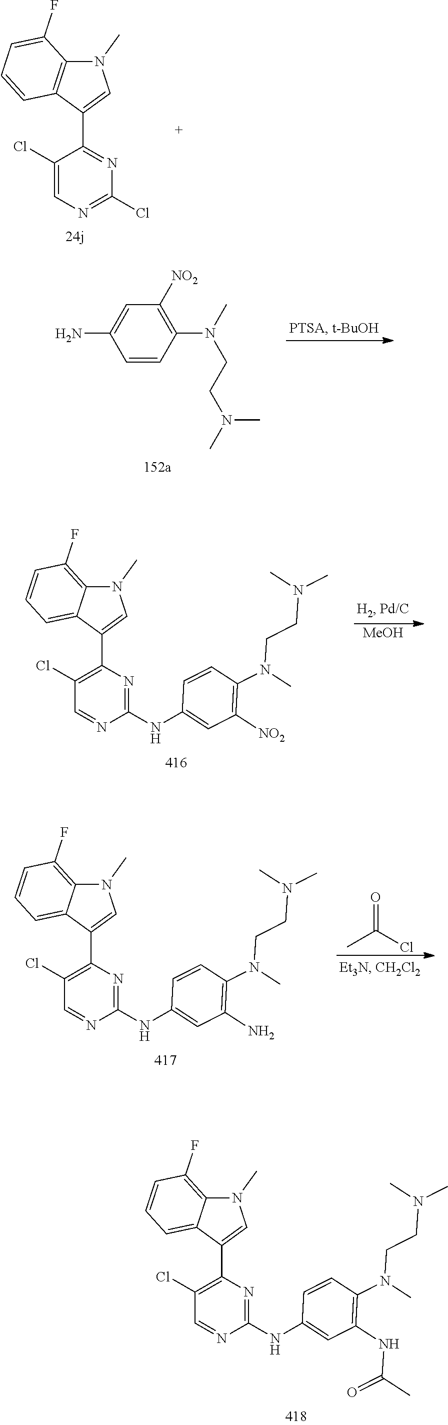

### N⁴-(5-chloro-4-(7-fluoro-1-methyl-1H-indol-3-yl)pyrimidin-2-yl)-N-(2-(dimethylamino)ethyl)-N¹-methyl-2-nitrobenzene-1,4-diamine (416)

A mixture of 24j (410 mg, 1.38 mmol), 152 (406 mg, 1.70 mmol), and PTSA (1.22 g, 7.08 mmol) in t-BuOH (50 mL) was stirred for 21 h at 80° C. The reaction was cooled to room temperature, and diluted with ethyl acetate (150 mL). The organic solution was washed with saturated NaHCO₃ (100 mL) and water (100 mL). Then organic phase was separated and concentrated under reduced pressure. The residue was purified by silica gel column (dichloromethane/methanol=20/1-10/1) to afford 416 (250 mg, yield 36.3%) as a red solid. MS-ESI (M+H)⁺: 498.4.

### N⁴-(5-chloro-4-(7-fluoro-1-methyl-1H-indol-3-yl)pyrimidin-2-yl)-N¹-(2-(dimethylamino)ethyl)-N¹-methylbenzene-1,2,4-triamine (417)

Pd/C (5% w/w, 25 mg, 0.01 mmol) was added into a solution of 416 (150 mg, 0.32 mmol) in methanol (20 mL) at 20° C. The resulting mixture was stirred at 40° C. under hydrogen atmosphere for 5 h. The reaction was then cooled to room temperature and filtered through celite. The filtrate was concentrated under reduced pressure. The residue was washed with dichloromethane and filtered to afford 417 (147 mg, yield 62.6%) as a yellow solid. ¹H NMR (500 MHz, DMSO) δ 9.34 (s, 1H), 8.53 (s, 1H), 8.46-8.44 (m, 1H), 8.41 (s, 1H), 7.07 (m, 3H), 7.01 (m, 1H), 6.90 (m, 2H), 4.09 (d, J=2.3 Hz, 3H), 3.32 (m, 9H), 3.06 (m, 2H), 2.67 (m, 2H). MS-ESI (M+H)⁺: 468.4

### N-(5-((5-chloro-4-(7-fluoro-1-methyl-1H-indol-3-yl)pyrimidin-2-yl)amino)-2-((2-(dimethylamino)ethyl)(methyl)amino)phenyl)acetamide (418)

Acetyl chloride (58 mg, 0.74 mmol) was added into a solution of 417 (120 mg, 0.26 mmol) and triethylamine (79 mg, 0.78 mmol) in dichloromethane (10 mL) at 20° C. The resulting mixture was stirred for 1.5 h. Then the reaction mixture was concentrated under reduced pressure. The residue was purified by preparative thin layer chromatography (dichloromethane/methanol=10/1) to afford 418 (41 mg, yield 31.4%) as a gray solid. MS-ESI (M+H)⁺: 510.4. ¹H NMR (500 MHz, CDCl₃) δ 9.82 (s, 1H), 8.45 (d, J=2.4 Hz, 1H), 8.35 (s, 1H), 8.28 (d, J=8.1 Hz, 1H), 8.17 (s, 1H), 7.66 (dd, J=8.6, 2.4 Hz, 1H), 7.26 (s, 1H), 7.10 (ddd, J=14.9, 10.2, 5.6 Hz, 2H), 6.95 (dt, J=19.8, 9.9 Hz, 1H), 4.09 (d, J=1.9 Hz, 3H), 3.03 (m, 2H), 2.70 (s, 3H), 2.63 (m, 2H), 2.48 (s, 6H), 2.29 (s, 3H).

### Procedure for the Synthesis of R-420

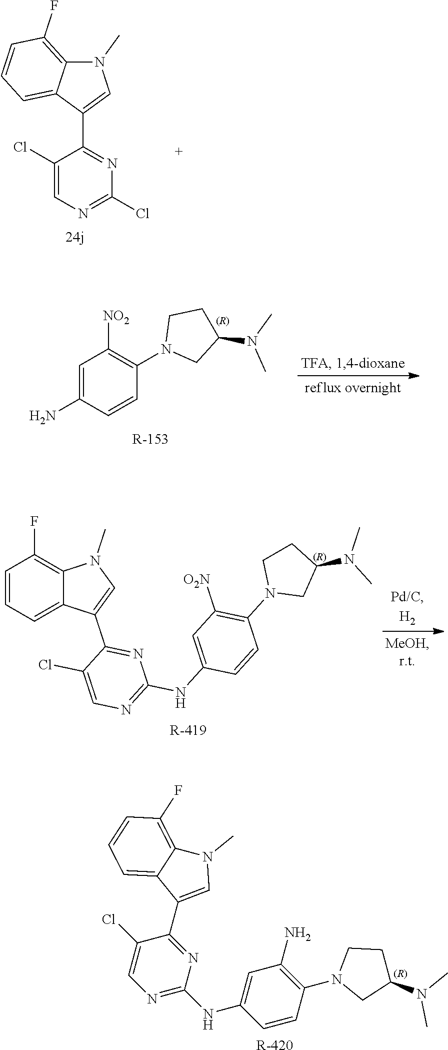

### (R)-5-chloro-N-(4-(3-(dimethylamino)pyrrolidin-1-yl)-3-nitrophenyl)-4-(7-fluoro-1-methyl-1H-indol-3-yl)pyrimidin-2-amine (R-419)

To the mixture of 24j (0.310 g, 1.0 mmol) and R-153 (0.250 g, 1.0 mmol) in 1,4-dioxane (20 mL) were added TFA (0.25 mL), then the mixture was refluxed overnight. After cooling to room temperature and concentration, the residue was purified by flash column chromatography (dichloromethane/methanol=30/1-20/1) to give crude, followed by recrystallization with ethanol to give R-419 as brownish red solid (100 mg, 19.2% yield). MS-ESI (M+H)⁺:522.4.

### (R)—N¹-(5-chloro-4-(7-methoxy-1-methyl-1H-indol-3-yl)pyrimidin-2-yl)-4-(3-(dimethylamino)pyrrolidin-1-yl)benzene-1,3-diamine (R-420)

To the mixture of R-419 (0.100 g, 0.196 mmol) in THF/methanol (20 mL/10 mL) were added Pd/C (5% w/w, 10 mg), then the mixture was stirred under H₂ at room temperature for 3 h. The reaction mixture was filtered through celite and concentrated under reduced pressure to give a residue. The residue was purified by flash column chromatography (dichloromethane/methanol=30/1-20/1) and preparative thin layer chromatography to give R-420 (6.8 mg, 5% yield) as a brown solid. MS-ESI (M+H)⁺: 480.2.

### Procedure for the Synthesis of S-420

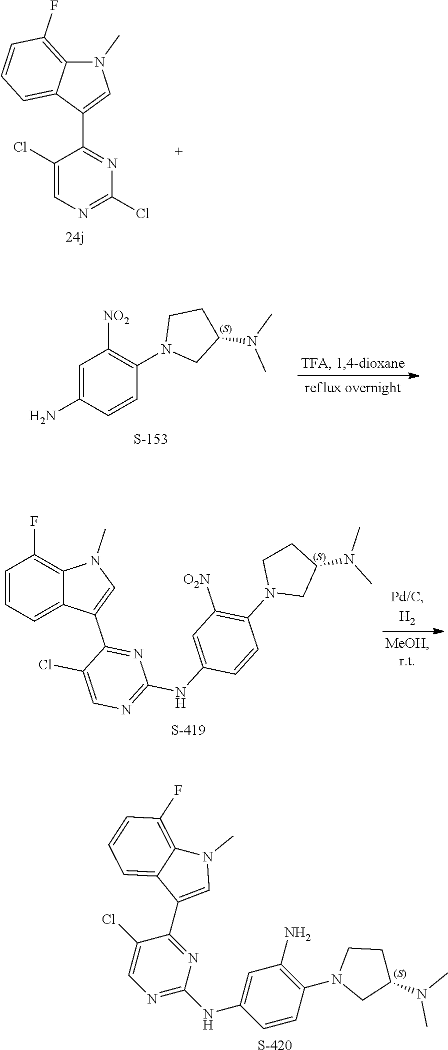

### (S)-5-chloro-N-(4-(3-(dimethylamino)pyrrolidin-1-yl)-3-nitrophenyl)-4-(7-fluoro-1-methyl-1H-indol-3-yl)pyrimidin-2-amine (S-419)

24j (0.25 g, 0.84 mmol), S-153 (0.23 g, 0.92 mmo), trifluoroacetic acid (0.25 g, 2.2 mmol) and 1,4-dioxane (20 ml) was added to the flask. The reaction mixture was stirred for 48 h at refluxing temperature. The reaction mixture was concentrated and the residue was dissolved with ethyl acetate (100 ml) and washed with water (50 ml*3). The organic layer was concentrated and the residue was purified by flash column chromatography (dichloromethane/methanol=20/1) to give the compound S-419 (100 mg, yield 23.38%). MS-ESI (M+H)⁺:510.3.

### (S)—N¹-(5-chloro-4-(7-fluoro-1-methyl-1H-indol-3-yl)pyrimidin-2-yl)-4-(3-(dimethylamino)pyrrolidin-1-yl)benzene-1,3-diamine (S-420)

The compound S-419 (100 mg, 0.19 mmol) was mixed with Pd/C (0.02 g) in methanol (10 ml). The resulting mixture was stirred at room temperature overnight under hydrogen atmosphere. The reaction mixture was filtered and concentrated. The residue was purified by flash column chromatography (dichloromethane/methanol=15/1) to give the compound S-420 (10 mg, yield 10.8%). MS-ESI (M+H)⁺:480.4.

### Procedure for the Syntheses of 422 and 423

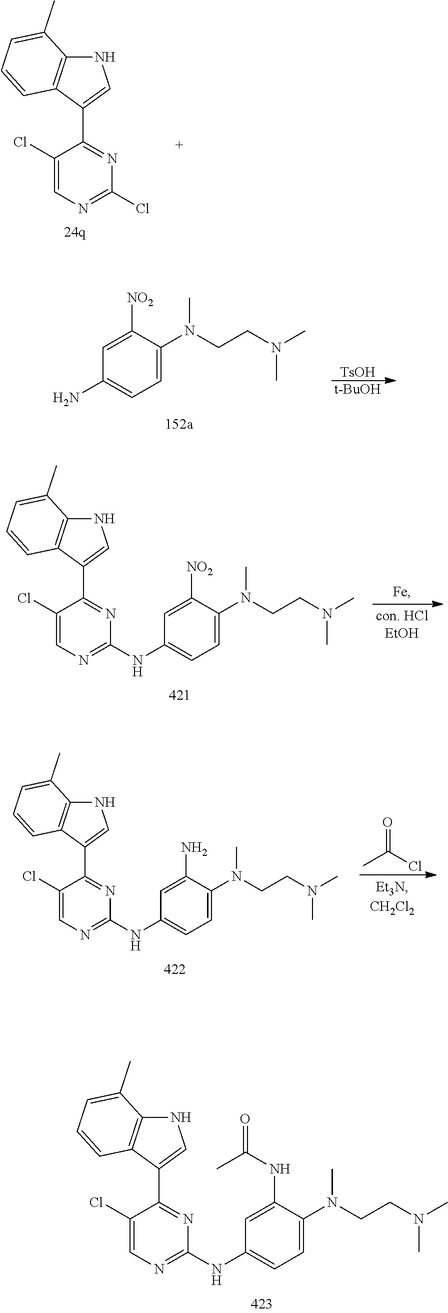

### N⁴-(5-chloro-4-(7-methyl-1H-indol-3-yl)pyrimidin-2-yl)-N¹-(2-(dimethylamino)ethyl)-N¹-methyl-2-nitrobenzene-1,4-diamine (421)

A mixture of 24q (509 mg, 1.83 mmol), 152a (417 mg, 1.75 mmol), and TsOH (629 mg, 3.65 mmol) in t-BuOH (20 mL) was stirred at 80° C. for 16 h. The reaction was cooled to 20° C. and diluted with dichloromethane (100 mL). The organic phase was washed with sat. NaHCO₃ (50 mL) and separated. Then organic solution was filtered through gel silica and concentrated to obtain crude product. The crude was slurried by ethyl acetate/petroleum ether (1/1, 20 mL) to afford 421 (550 mg, yield 62.6%) as a black solid. MS-ESI (M+H)⁺: 480.4.

### N⁴-(5-chloro-4-(7-methyl-1H-indol-3-yl)pyrimidin-2-yl)-N¹-(2-(dimethylamino)ethyl)-N¹-methylbenzene-1,2,4-triamine (422)

A mixture of 421 (404 mg, 0.84 mmol) and con. HCl (0.5 mL, 6.00 mL) in ethanol (20 mL) was added iron powder (452 mg, 8.09 mmol) at 20° C. Then the reaction was stirred at 80° C. for 4 h. The reaction was then cooled to 20° C. and quenched with saturated NaHCO₃ (20 mL). The reaction was filtered and concentrated under reduced pressure to give a crude product. The residue was slurried with dichloromethane and filtered to afford 422 (100 mg, yield 26.4%) as gray solid. ¹H NMR (500 MHz, DMSO) δ 11.96 (d, J=2.2 Hz, 1H), 9.30 (s, 1H), 8.45 (d, J=7.3 Hz, 1H), 8.43 (d, J=3.1 Hz, 1H), 8.38 (s, 1H), 7.07-6.99 (m, 3H), 6.94-6.88 (m, 2H), 3.19 (m, 2H), 3.12 (m, 2H), 2.72 (s, 5H), 2.52 (s, 3H), 2.50 (s, 3H). MS-ESI (M+H)⁺: 450.3

### N-(5-((5-chloro-4-(7-methyl-1H-indol-3-yl)pyrimidin-2-yl)amino)-2-((2-(dimethylamino)ethyl)(methyl)amino)phenyl)acetamide (423)

A solution of 422 (30 mg, 0.07 mmol) and Et₃N (100 mg, 0.99 mmol) in dichloromethane (10 mL) was added CH₃COCl (35 mg, 0.45 mmol) under 20° C. and the reaction was stirred for 1.5 h. The reaction was quenched with sat. NaHCO₃ (30 mL) and diluted with dichloromethane (50 mL). The organic phase was washed with water (30 mL), brine (30 mL), dried over Na₂SO₄ and concentrated under reduced pressure to give the residue. The residue was purified by preparative thin layer chromatography (dichloromethane/methanol=10/1) to afford 423 (5 mg, yield 15.2%) as white solid. ¹H NMR (500 MHz, DMSO) δ 11.95 (s, 1H), 9.83 (s, 1H), 9.57 (s, 1H), 8.45 (d, J=3.1 Hz, 1H), 8.41 (s, 1H), 8.36 (s, 1H), 7.53 (dd, J=8.6, 2.3 Hz, 1H), 7.19 (d, J=8.7 Hz, 1H), 7.05-6.99 (m, 2H), 3.02-2.98 (m, 2H), 2.61 (s, 3H), 2.52 (s, 3H), 2.47 (m, 2H), 2.44 (s, 3H), 2.13 (s, 6H). MS-ESI (M+H)⁺: 492.4

### Procedure for the Syntheses of R-425 and R-426

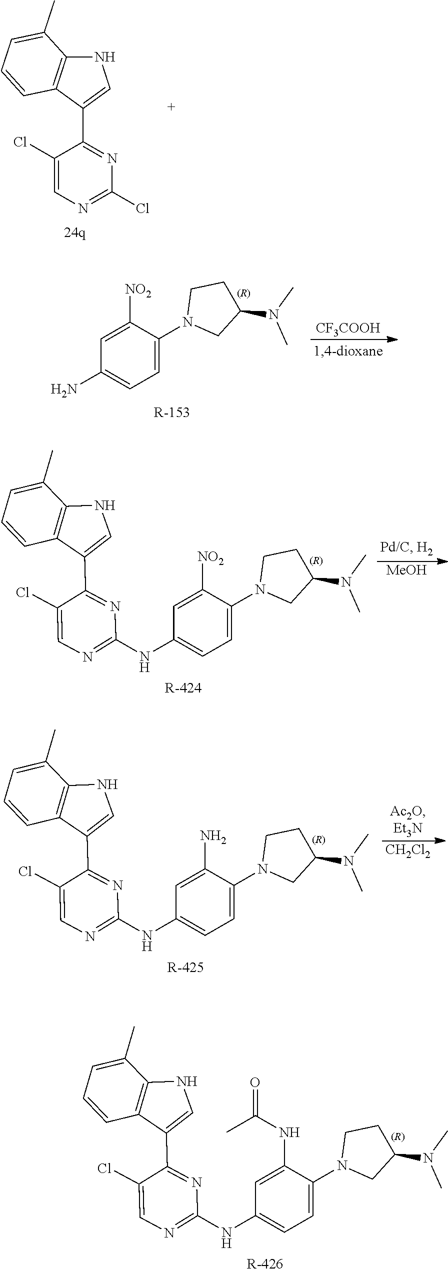

### (R)-5-chloro-N-(4-(3-(dimethylamino)pyrrolidin-1-yl)-3-nitrophenyl)-4-(7-methyl-1H-indol-3-yl)pyrimidin-2-amine (R-424)

A mixture of 24q (348 mg, 1.25 mmol), R-153 (320 mg, 1.28 mmol), and CF₃COOH (441 mg, 3.87 mmol) in 1,4-dioxane (20 mL) was stirred at 80° C. for 22 h. The reaction was cooled to room temperature and diluted with dichloromethane/methanol solution (10/1, 50 mL). The organic solution was filtered through gel silica to obtain the crude product. The crude was purified by preparative thin layer chromatography (dichloromethane/methanol=10/1) to afford R-424 (150 mg, yield 24.4%) as red solid. MS-ESI (M+H)⁺: 492.4.

### (R)—N¹-(5-chloro-4-(7-methyl-1H-indol-3-yl)pyrimidin-2-yl)-4-(3-(dimethylamino)pyrrolidin-1-yl)benzene-1,3-diamine (R-425)

Pd/C (5% w/w, 50 mg) was added into a solution of R-424 (150 mg, 0.30 mmol) in methanol (20 mL) at 10° C. Then reaction was stirred under H₂ atmosphere for 16 h at 10° C. Then the reaction was filtered through celite and concentrated under reduced pressure to give a residue. The residue was purified by preparative thin layer chromatography (dichloromethane/methanol=10/1) to afford R-425 (28 mg, yield 19.9%) as yellow solid. ¹H NMR (500 MHz, DMSO) δ 11.84 (s, 1H), 9.23 (s, 1H), 8.42 (t, J=5.7 Hz, 2H), 8.37 (s, 1H), 7.04 (dt, J=14.5, 4.8 Hz, 3H), 6.93 (dd, J=8.5, 2.3 Hz, 1H), 6.86 (d, J=8.5 Hz, 1H), 3.24-3.08 (m, 4H), 2.88-2.81 (m, 1H), 2.77 (s, 6H), 2.52 (s, 3H), 2.27 (m, 1H), 2.04 (m, 1H). MS-ESI (M+H)⁺: 462.5

### (R)—N-(5-((5-chloro-4-(7-methyl-1H-indol-3-yl)pyrimidin-2-yl)amino)-2-(3-(dimethylamino)pyrrolidin-1-yl)phenyl)acetamide (R-426)

CH₃COCl (50 mg, 0.49) was added into a solution of R-425 (20 mg, 0.04 mmol) and Et₃N (50 mg, 0.49 mmol) in dichloromethane (5 mL) at 20° C. Then reaction was stirred for 4 h. The reaction was quenched with methanol and concentrated under reduced pressure to give a residue. The residue was purified by preparative thin layer chromatography (dichloromethane/methanol=10/1) to afford R-426 (10 mg, yield 45.83%) as white solid. MS-ESI (M+H)⁺: 504.5

### Procedure for the Syntheses of S-425 and S-426

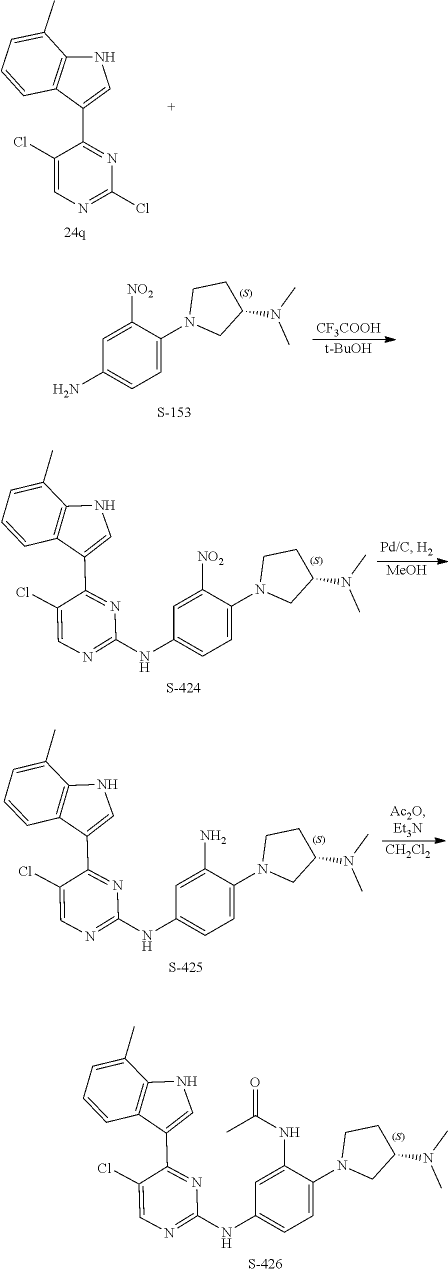

### (S)-5-chloro-4-(1,7-dimethyl-1H-indol-3-yl)-N-(4-(3-(dimethylamino)pyrrolidin-1-yl)-3-nitrophenyl)pyrimidin-2-amine (S-424)

A mixture of 24q (506 mg, 1.73 mmol), S-153 (410 mg, 1.64 mmol), and TsOH (610 mg, 3.54 mmol) in t-BuOH (20 mL) and 1,4-dioxane (10 mL) was stirred at 80° C. for 70 h. The reaction was cooled to room temperature and diluted with dichloromethane (100 mL). The organic solution was filtered through celite and concentrated under reduced pressure to give a residue. The residue was slurried by petroleum ether/ethyl acetate (1/1, 20 mL) to afford S-424 (150 mg, yield 17.1%) as gray solid.

### (S)—N¹-(5-chloro-4-(1,7-dimethyl-1H-indol-3-yl)pyrimidin-2-yl)-4-(3-(dimethylamino)pyrrolidin-1-yl)benzene-1,3-diamine (S-425)

Pd/C (5% w/w, 100 mg) was added into a solution of S-424 (150 mg, 0.30 mmol) in methanol (20 mL) at 10° C. Then reaction was stirred under H₂ atmosphere for 16 h at 10° C. Then the reaction was filtered through celite and concentrated under reduced pressure to give a residue. The residue was purified by preparative thin layer chromatography (dichloromethane/methanol=10/1) to afford S-425 (40 mg, yield 28.4%) as yellow solid. MS-ESI (M+H)⁺: 462.5.

### (S)—N-(5-((5-chloro-4-(1,7-dimethyl-1H-indol-3-yl)pyrimidin-2-yl)amino)-2-(3-(dimethylamino)pyrrolidin-1-yl)phenyl)acetamide (S-426)

CH₃COCl (50 mg, 0.49) was added into a solution of S-425 (25 mg, 0.06 mmol) and Et₃N (50 mg, 0.49 mmol) in dichloromethane (10 mL) at 20° C. Then reaction was stirred for 4 h. The reaction was quenched with methanol and concentrated under reduced pressure to give a residue. The residue was purified by preparative thin layer chromatography (dichloromethane/methanol=10/1) to afford S-426 (10 mg, yield 36.8%) as white solid. MS-ESI (M+H)⁺: 504.5

### Procedure for the Syntheses of 428 and 429

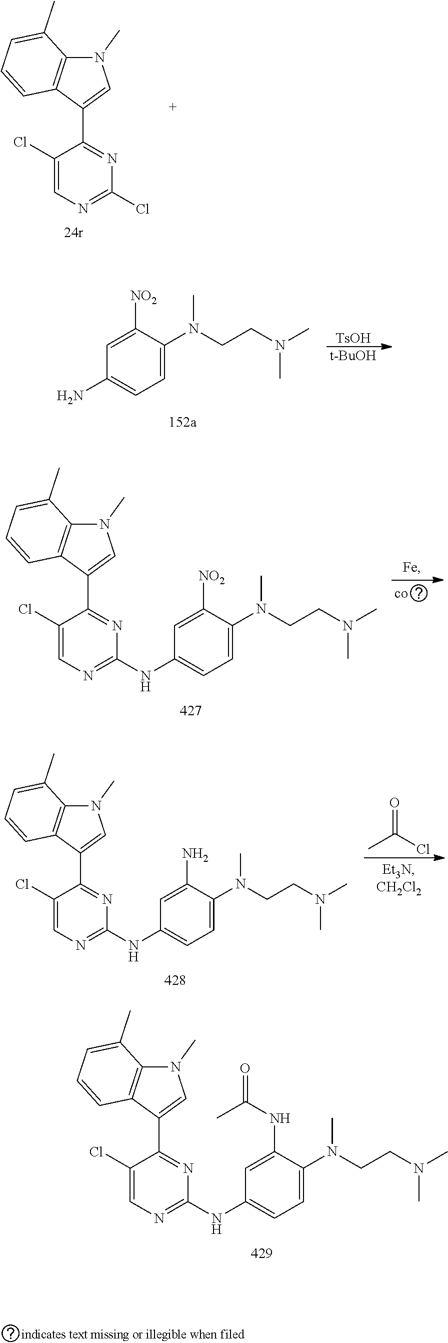

### N⁴-(5-chloro-4-(1,7-dimethyl-1H-indol-3-yl)pyrimidin-2-yl)-N¹-(2-(dimethylamino)ethyl)-N¹-methyl-2-nitrobenzene-1,4-diamine (427)

A mixture of 24r (505 mg, 1.73 mmol), 152a (396 mg, 1.66 mmol), and TsOH (612 mg, 3.55 mmol) in t-BuOH (20 mL) was stirred at 80° C. for 21 h. The reaction was cooled to 20° C. and diluted with dichloromethane (100 mL). The organic phase was washed with sat. NaHCO₃ (100 mL) and water (100 mL). Then organic solution was filtered through gel silica and concentrated to obtain crude product. The residue was slurried by ethyl acetate/petroleum ether (1/1, 20 mL) to afford 427 (680 mg, yield 80.4%) as orange solid. MS-ESI (M+H)⁺: 494.4.

### N⁴-(5-chloro-4-(1,7-dimethyl-1H-indol-3-yl)pyrimidin-2-yl)-N¹-(2-(dimethylamino)ethyl)-N¹-methylbenzene-1,2,4-triamine (428)

A mixture of 427 (401 mg, 0.81 mmol) and con. HCl (0.5 mL, 6.00 mL) in ethanol (20 mL) was added iron powder (446 mg, 7.99 mmol) at 20° C. Then the reaction was stirred at 80° C. for 4 h. The reaction was then cooled to 20° C. and quenched with sat NaHCO₃ (20 mL). The reaction was filtered and concentrated under reduced pressure to give a crude product. The residue was slurried with methanol and filtered to afford 428 (200 mg, yield 53.1%) as yellow solid. ¹H NMR (500 MHz, CDCl₃) δ 8.47 (d, J=8.0 Hz, 1H), 8.30 (s, 1H), 8.09 (s, 1H), 7.32 (d, J=2.4 Hz, 1H), 7.12-7.07 (m, 1H), 7.00 (dd, J=7.2, 3.3 Hz, 3H), 6.78 (dd, J=8.4, 2.5 Hz, 1H), 4.15 (s, 3H), 2.94 (t, J=6.7 Hz, 2H), 2.80 (s, 3H), 2.67 (s, 3H), 2.41 (t, J=6.7 Hz, 2H), 2.26 (s, 6H). MS-ESI (M+H)⁺: 464.3.

### N-(5-((5-chloro-4-(1,7-dimethyl-1H-indol-3-yl)pyrimidin-2-yl)amino)-2-((2-(dimethylamino)ethyl)(methyl)amino)phenyl)acetamide (429)

A solution of 428 (50 mg, 0.11 mmol) and Et₃N (100 mg, 0.99 mmol) in dichloromethane (10 mL) was added CH₃COCl (30 mg, 0.38 mmol) under 20° C. and the reaction was stirred for 1.5 h. The reaction was quenched with sat. NaHCO₃ (30 mL) and diluted with dichloromethane (50 mL). The organic phase was washed with water (30 mL), brine (30 mL), dried over Na₂SO₄ and concentrated under reduced pressure to give the residue. The residue was purified by preparative thin layer chromatography (dichloromethane/methanol=10/1) to afford 429 (13 mg, yield 23.8%) as white solid. ¹H NMR (500 MHz, CDCl₃) δ 9.92 (s, 1H), 8.52 (s, 1H), 8.49 (d, J=2.3 Hz, 1H), 8.20 (s, 1H), 7.92 (d, J=7.3 Hz, 1H), 7.24 (d, J=8.4 Hz, 1H), 7.07 (dd, J=8.4, 2.5 Hz, 1H), 6.95-6.88 (m, 2H), 4.09 (s, 3H), 2.93 (m, 2H), 2.74 (s, 3H), 2.73 (s, 3H), 2.51 (m, 2H), 2.38 (s, 3H), 2.36 (s, 6H). MS-ESI (M+H)⁺: 506.3

### Procedure for the Syntheses of R-431 and R-432

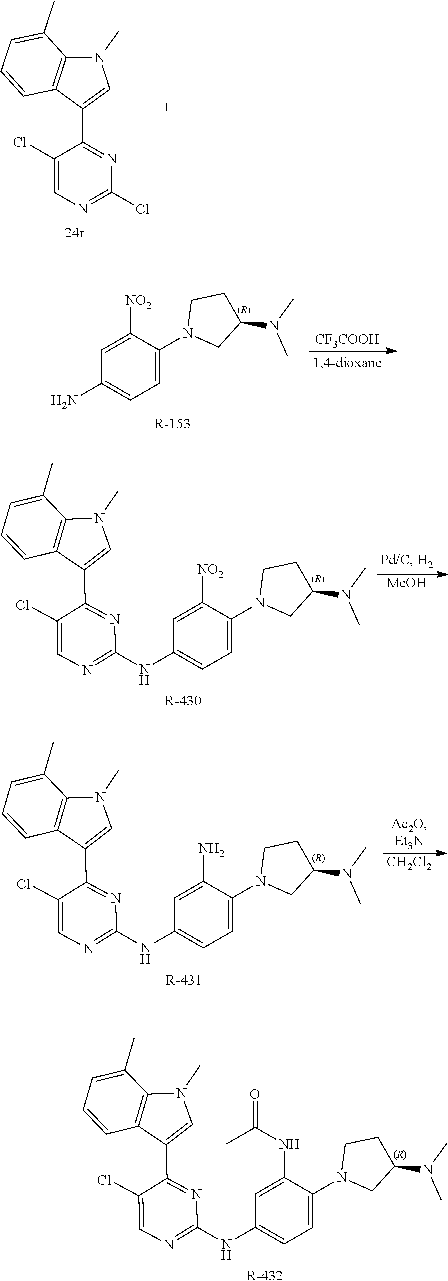

### (R)-5-chloro-4-(1,7-dimethyl-1H-indol-3-yl)-N-(4-(3-(dimethylamino)pyrrolidin-1-yl)-3-nitrophenyl)pyrimidin-2-amine (R-430)

A mixture of 24r (353 mg, 1.21 mmol), R-153 (308 mg, 1.23 mmol), and CF₃COOH (419 mg, 3.67 mmol) in 1,4-dioxane (10 mL) was stirred at 80° C. for 18 h. The reaction was cooled to room temperature and diluted with dichloromethane (100 mL). The organic solution was washed with sat. NaHCO₃ (100 mL) and water (100 mL). Then organic phase was concentrated under reduced pressure to give a residue. The residue was purified by flash column chromatography (dichloromethane/methanol=50/1-20/1) to afford R-430 (206 mg, yield 33.7%) as red solid. MS-ESI (M+H)⁺: 506.2.

### (R)—N¹-(5-chloro-4-(1,7-dimethyl-1H-indol-3-yl)pyrimidin-2-yl)-4-(3-(dimethylamino)pyrrolidin-1-yl)benzene-1,3-diamine (R-431)

Pd/C (5% w/w, 80 mg) was added into a solution of R-430 (206 mg, 0.41 mmol) in methanol (20 mL) at 10° C. Then reaction was stirred under H₂ atmosphere for 16 h at 10° C. Then the reaction was filtered through celite and concentrated under reduced pressure to give a residue. The residue was purified by preparative thin layer chromatography (dichloromethane/methanol=10/1) to afford R-431 (60 mg, yield 31.0%) as yellow solid. ¹H NMR (500 MHz, DMSO) δ 9.22 (s, 1H), 8.48 (d, J=7.4 Hz, 1H), 8.38 (s, 1H), 8.36 (s, 1H), 7.05 (d, J=2.3 Hz, 1H), 7.03-6.95 (m, 2H), 6.91 (dd, J=8.5, 2.4 Hz, 1H), 6.85 (d, J=8.5 Hz, 1H), 4.18 (s, 3H), 3.09 (m, 4H), 2.94 (m, 1H), 2.78 (s, 3H), 2.59-2.53 (m, 6H), 2.22-2.13 (m, 1H), 2.05-1.93 (m, 1H). MS-ESI (M+H)⁺: 476.5.

### (R)—N-(5-((5-chloro-4-(1,7-dimethyl-1H-indol-3-yl)pyrimidin-2-yl)amino)-2-(3-(dimethylamino)pyrrolidin-1-yl)phenyl)acetamide (R-432)

CH₃COCl (50 mg, 0.49) was added into a solution of R-431 (30 mg, 0.06 mmol) and Et₃N (50 mg, 0.49 mmol) in dichloromethane (10 mL) at 20° C. Then reaction was stirred for 4 h. The reaction was quenched with methanol and concentrated under reduced pressure to give a residue. The residue was purified by preparative thin layer chromatography (dichloromethane/methanol=10/1) to afford R-432 (20 mg, yield 61.3%) as white solid. ¹H NMR (500 MHz, CDCl₃) δ 9.06 (s, 1H), 8.36 (d, J=8.0 Hz, 1H), 8.30 (s, 1H), 8.27 (s, 1H), 8.09 (s, 1H), 7.67 (d, J=6.5 Hz, 1H), 7.12 (s, 1H), 7.07 (dd, J=14.3, 6.4 Hz, 1H), 6.99 (t, J=8.3 Hz, 2H), 4.15 (s, 3H), 3.60 (m, 4H), 3.23 (m, 1H), 2.84 (s, 6H), 2.78 (s, 3H), 2.41 (s, 3H), 2.33 (m, 2H). MS-ESI (M+H)⁺: 518.5.

### Procedure for the Synthesis of S-431

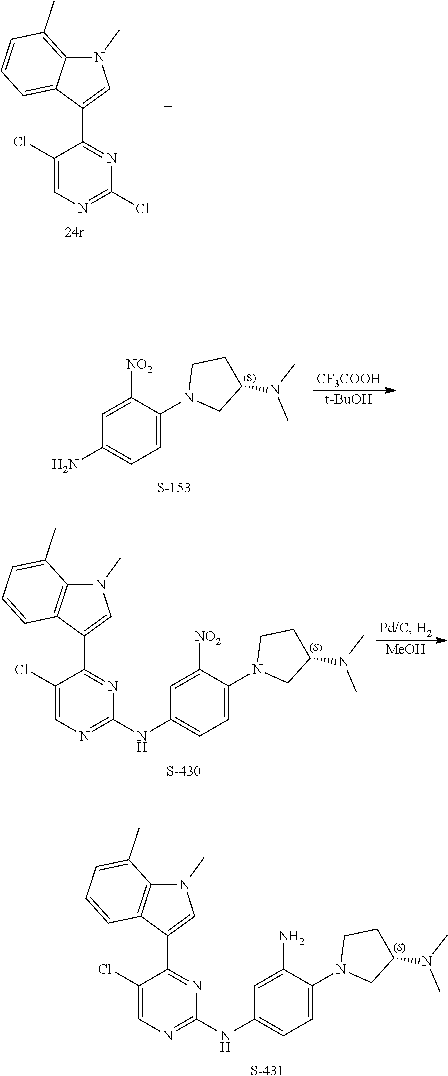

### (S)-5-chloro-4-(1,7-dimethyl-1H-indol-3-yl)-N-(4-(3-(dimethylamino)pyrrolidin-1-yl)-3-nitrophenyl)pyrimidin-2-amine (S-430)

A mixture of 24r (401 mg, 1.37 mmol), S-153 (303 mg, 1.21 mmol), and TsOH (498 mg, 2.89 mmol) in t-BuOH (20 mL) was stirred at 80° C. for 70 h. The reaction was cooled to 20° C. and diluted with dichloromethane (100 mL). The organic phase was washed with sat. NaHCO₃ (50 mL) and concentrated to obtain crude product. The residue was purified by flash column chromatography (dichloromethane/methanol=100/1-10/1) to afford S-430 (202 mg, yield 29.09%) as red solid. MS-ESI (M+H)⁺: 506.2

### (S)—N-(5-chloro-4-(1,7-dimethyl-1H-indol-3-yl)pyrimidin-2-yl)-4-(3-(dimethylamino)pyrrolidin-1-yl)benzene-1,3-diamine (S-431)

A mixture of S-430 (201 mg, 0.40 mmol) and con. HCl (0.5 mL, 6.00 mmoL) in ethanol (20 mL) was added iron powder (214 mg, 3.83 mmol) at 20° C. Then the reaction was stirred at 80° C. for 4 h. The reaction was then cooled to 20° C. The mixture was quenched with saturated NaHCO₃ (50 mL), diluted with dichloromethane (100 mL). Then organic solution was separated and filtered through gel silica to give a crude product. The residue was purified by preparative thin layer chromatography to afford S-431 (20 mg, yield 10.6%) as yellow solid. ¹H NMR (500 MHz, DMSO) δ 9.25 (s, 1H), 8.48 (d, J=7.6 Hz, 1H), 8.39 (s, 1H), 8.36 (s, 1H), 7.08-6.89 (m, 4H), 6.84 (d, J=8.5 Hz, 1H), 4.17 (s, 3H), 3.19-3.04 (m, 4H), 2.85 (m, 1H), 2.77 (s, 3H), 2.69 (s, 6H), 2.22 (m, 1H), 2.04 (m, 1H). MS-ESI (M+H)⁺: 476.5

### Procedure for the Synthesis of S-432

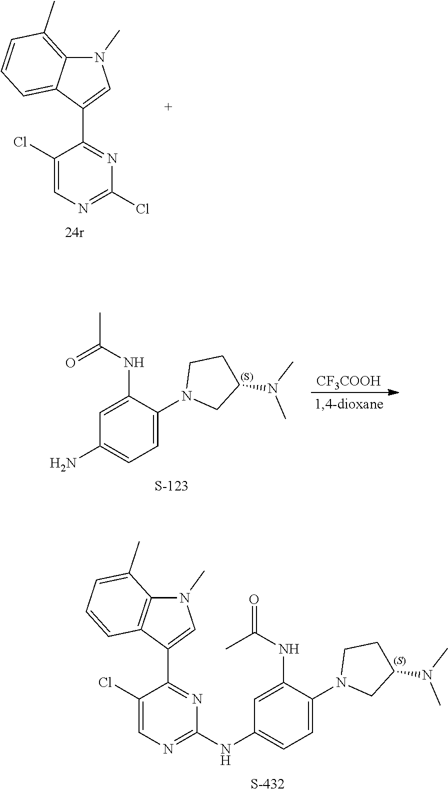

### (S)—N-(5-((5-chloro-4-(1,7-dimethyl-1H-indol-3-yl)pyrimidin-2-yl)amino)-2-(3-(dimethylamino)pyrrolidin-1-yl)phenyl)acetamide (S-432)

A mixture of 24r (101 mg, 0.35 mmol), S-123 (65 mg, 0.25 mmol), and CF₃COOH (89 mg, 0.78 mmol) in 1,4-dioxane (10 mL) was stirred at 80° C. for 20 h. The reaction was cooled to 20° C. and diluted with dichloromethane (100 mL). The organic phase was washed with sat. NaHCO₃ (50 mL) and concentrated to obtain crude product. The residue was purified by preparative thin layer chromatography (dichloromethane/methanol=10/1) to afford S-432 (35 mg, yield 19.54%) as white solid. ¹H NMR (500 MHz, CDCl₃) δ 8.96 (s, 1H), 8.37 (d, J=8.0 Hz, 1H), 8.31 (s, 1H), 8.28 (d, J=1.9 Hz, 1H), 8.10 (s, 1H), 7.67-7.64 (m, 1H), 7.18 (s, 1H), 7.08 (t, J=7.6 Hz, 1H), 6.99 (m, 2H), 4.14 (s, 3H), 3.62-3.36 (m, 4H), 2.83 (m, 1H), 2.78 (s, 3H), 2.73 (s, 6H), 2.38 (s, 3H), 2.35-2.26 (m, 2H). MS-ESI (M+H)⁺: 518.4.

### Procedure for the Synthesis of 433

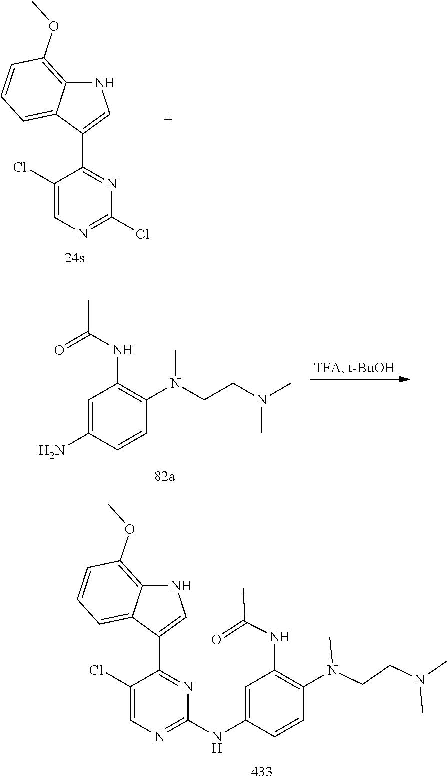 [SMILES: COc1cccc2c(-c3nc(Cl)ncc3Cl)c[nH]c12 ; CC(=O)Nc1cc(N)ccc1N(C)CCN(C)C ; COc1cccc2c(-c3nc(Nc4ccc(N(C)CCN(C)C)c(NC(C)=O)c4)ncc3Cl)c[nH]c12]

### N-(5-(5-chloro-4-(7-methoxy-1H-indol-3-yl)pyrimidin-2-ylamino)-2-((2-(dimethylamino)ethyl)(methyl)amino)phenyl)acetamide (433)

To the mixture of 24s (0.403 g, 1.38 mmol) and 82a (0.320 g, 1.28 mmol) in t-BuOH (30 mL) was added TFA (0.2 mL). The mixture was stirred at refluxing temperature for 2 days. After cooling to room temperature and concentration, the residue was purified by flash column chromatography (dichloromethane/methanol=30/1-20/1) and preparative thin layer chromatography to give 433 (13 mg, 1.17% yield) as a gray solid. MS-ESI (M+H)⁺: 508.3.

### Procedure for the Synthesis of 434

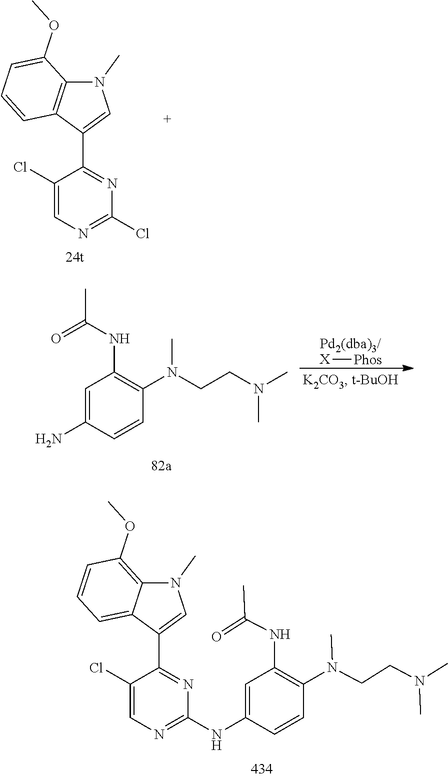

### N-(5-(5-chloro-4-(7-methoxy-1-methyl-1H-indol-3-yl)pyrimidin-2-ylamino)-2-((2-(dimethylamino)ethyl)(methyl)amino)phenyl)acetamide (434)

To the mixture of 24t (0.150 g, 0.49 mmol) and 82a (0.125 g, 0.50 mmol) in t-BuOH (30 mL) were added K₂CO₃ (0.301 g, 2.18 mmol), Pd₂(dba)₃ (85 mg, 0.095 mmol) and X-phos (80 mg, 0.17 mmol). The mixture was stirred at 85° C. overnight. After cooling to room temperature and concentration, the residue was purified by flash column chromatography (dichloromethane/methanol=30/1-20/1) and preparative thin layer chromatography to give 434 (15 mg, 6% yield) as a brown solid. MS-ESI (M+H)⁺: 522.

### Procedure for the Synthesis of R-435

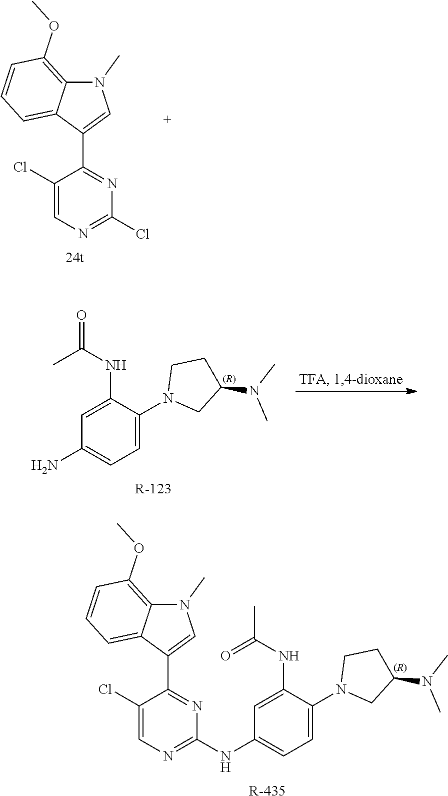

### (R)—N-(5-(5-chloro-4-(7-methoxy-1-methyl-1H-indol-3-yl)pyrimidin-2-ylamino)-2-(3-(dimethylamino)pyrrolidin-1-yl)phenyl)acetamide (R-435)

To the mixture of 24t (0.090 g, 0.293 mmol) and R-123 (0.100 g, 0.383 mmol) in 1,4-dioxane (20 mL) were added TFA (0.2 mL), the mixture was refluxed for 2 days. After cooling to room temperature and concentration, the residue was purified by flash column chromatography (dichloromethane/methanol=30/1-20/1) and preparative thin layer chromatography to give R-435 (25 mg, 12.3% yield) as a brown solid. MS-ESI (M+H)⁺: 534.4.

### Procedure for the Synthesis of S-435

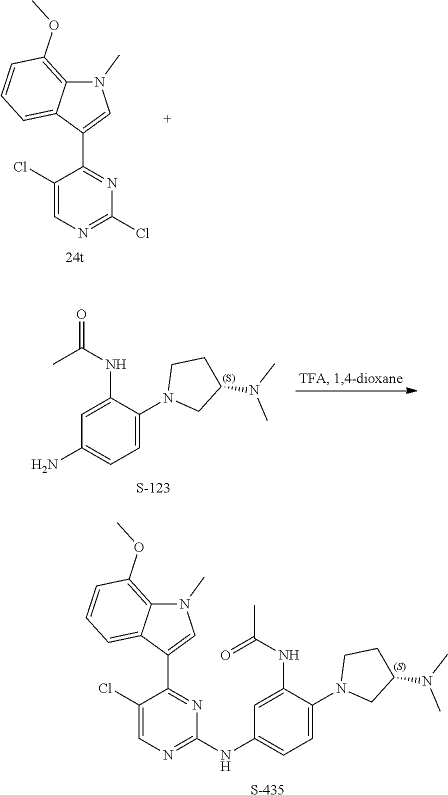

### (S)—N-(5-(5-chloro-4-(7-methoxy-1-methyl-1H-indol-3-yl)pyrimidin-2-ylamino)-2-(3-(dimethylamino)pyrrolidin-1-yl)phenyl)acetamide (S-435)

To the mixture of 24t (0.180 g, 0.586 mmol) and S-123 (0.133 g, 0.510 mmol) in 1,4-dioxane (30 mL) were added TFA (0.3 mL), the mixture was refluxed for 2 days. After cooling to room temperature and concentration, the residue was purified by flash column chromatography (dichloromethane/methanol=30/1-20/1) and preparative thin layer chromatography to give S-435 (17 mg, 6.3% yield) as a brown solid. MS-ESI (M+H)⁺: 534.4.

### Procedure for the Synthesis of 437

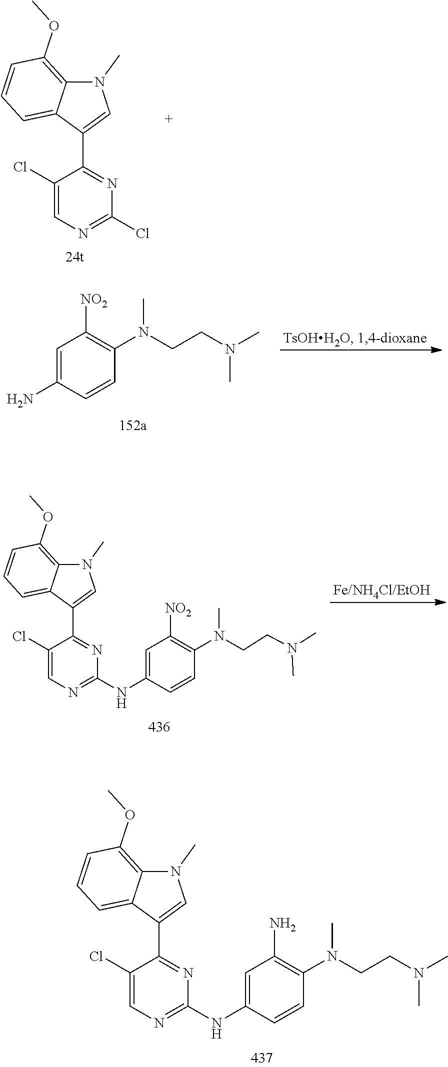 [SMILES: COc1cccc2c(-c3nc(Cl)ncc3Cl)cn(C)c12 ; CN(C)CCN(C)c1ccc(N)cc1[N+](=O)[O-] ; COc1cccc2c(-c3nc(Nc4ccc(N(C)CCN(C)C)c([N+](=O)[O-])c4)ncc3Cl)cn(C)c12 ; COc1cccc2c(-c3nc(Nc4ccc(N(C)CCN(C)C)c(N)c4)ncc3Cl)cn(C)c12]

### N⁴-(5-chloro-4-(7-methoxy-1-methyl-1H-indol-3-yl)pyrimidin-2-yl)-N¹-(2-(dimethylamino)ethyl)-N¹-methyl-2-nitrobenzene-1,4-diamine (436)

To the mixture of 24t (0.236 g, 0.98 mmol) and 152a (0.236 g, 0.99 mmol) in 1,4-dioxane (20 mL) was added TsOH H₂ (0.300 g, 1.50 mmol), then the mixture was refluxed for 2 days. After cooling to room temperature and concentration, the residue was purified by flash column chromatography (dichloromethane/methanol 30/1-20/1) to give crude 436 (350 mg, 70% yield) as brownish red solid. MS-ES (M+H)⁺: 510.3.

### N⁴-(5-chloro-4-(7-methoxy-1-methyl-H-indol-3-yl)pyrimidin-2-yl)-N¹-(2-(dimethylamino)ethyl)-N¹-methylbenzene-1,2,4-triamine (437)

To the mixture of 436 (0.300 g, 0.589 mmol) in solution ethanol/1H₂O (30 mL/10 mL) was added iron powder (0.260 g, 4.64 mmol) and NH₄C1 (0.500 g, 9.35 mmol), then the mixture was refluxed for 2 h. After cooling to room temperature and concentration, the residue was extracted by dichloromethane, washed with brine and concentrated followed by purifying through preparative thin layer chromatography to give 437 (40 mg, 14% yield) as a brown solid. MS-ESI (M+H)⁺: 480.0.

### Procedure for the Synthesis of R-439

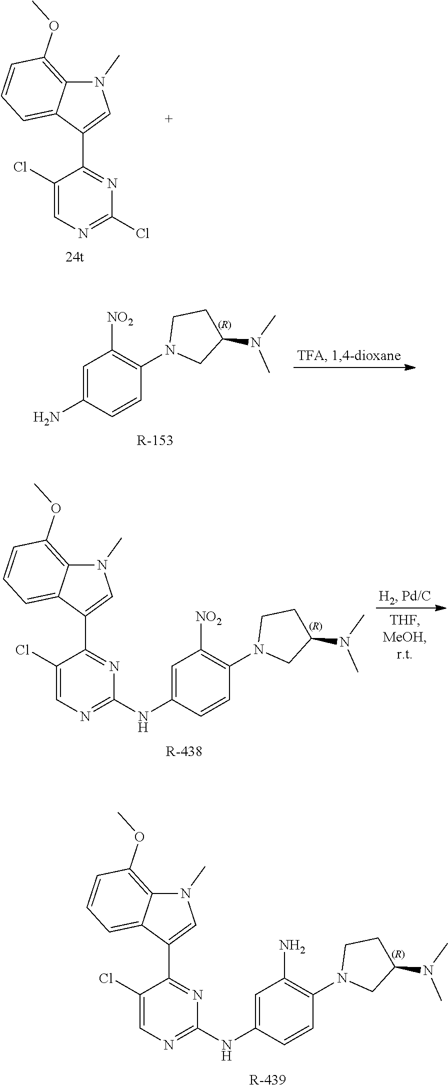

### (R)-5-chloro-N-(4-(3-(dimethylamino)pyrrolidin-1-yl)-3-nitrophenyl)-4-(7-methoxy-1-methyl-1H-indol-3-yl)pyrimidin-2-amine (R-438)

To the mixture of 24t (0.310 g, 1.0 mmol) and R-153 (0.250 g, 1.0 mmol) in 1,4-dioxane (20 mL) was added TFA (0.25 mL), then the mixture was refluxed overnight. After cooling to room temperature and concentration, the residue was purified by flash column chromatography (dichloromethane/methanol=30/1-20/1) to give crude, followed by recrystallization with ethanol to give R-438 as brownish red solid (100 mg, 19.2% yield). MS-ESI (M+H)⁺:522.4.

### (R)—N¹-(5-chloro-4-(7-methoxy-1-methyl-1H-indol-3-yl)pyrimidin-2-yl)-4-(3-(dimethylamino)pyrrolidin-1-yl)benzene-1,3-diamine (R-439)

To the mixture of R-438 (0.100 g, 0.192 mmol) in THF/methanol (20 mL/10 mL) were added Pd/C (5% w/w, 55 mg), then the mixture was stirred under H₂ atmosphere for 3 h at room temperature. The reaction mixture was filtered through celite and concentrated under reduced pressure to give a residue. The residue was purified by flash column chromatography (dichloromethane/methanol=30/1-20/1) and preparative thin layer chromatography to give R-439 (5 mg, 0.010 mmol, 5% yield) as a brown solid. MS-ESI (M+H)⁺: 492.5. ¹H NMR (500 MHz, CDCl3) δ 8.29 (s, 1H), 8.17 (d, J=8.1 Hz, 1H), 8.07 (s, 1H), 7.35 (d, J=1.9 Hz, 1H), 7.11 (t, J=8.0 Hz, 1H), 7.02-6.92 (m, 2H), 6.79 (dd, J=8.4, 2.2 Hz, 1H), 6.70 (d, J=7.7 Hz, 1H), 5.35 (t, J=4.7 Hz, 1H), 4.14 (s, 3H), 3.95 (s, 3H), 3.30-3.01 (m, 5H), 2.47 (s, 6H), 2.27-2.15 (m, 2H).

### Procedure for the Synthesis of S-439

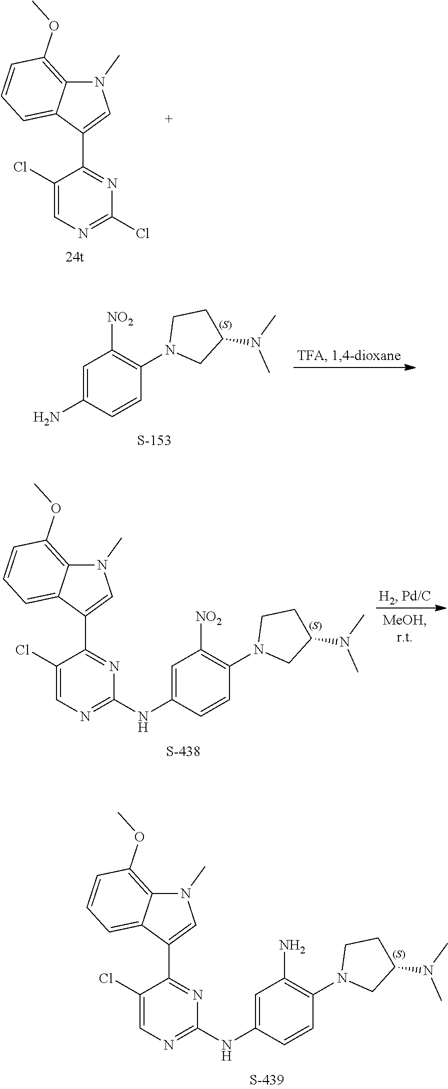

### (S)-5-chloro-N-(4-(3-(dimethylamino)pyrrolidin-1-yl)-3-nitrophenyl)-4-(7-methoxy-1-methyl-1H-indol-3-yl)pyrimidin-2-amine (S-438)

Synthesis of S-438 was similar to that of R-438. The residue was purified by preparative thin layer chromatography (dichloromethane/methanol=15/1) to give the title product S-438 (50 mg, yield 6.0%) as a gray solid. MS-ESI (M+H)⁺:522.4.

### (S)—N¹-(5-chloro-4-(7-methoxy-1-methyl-1H-indol-3-yl)pyrimidin-2-yl)-4-(3-(dimethylamino)pyrrolidin-1-yl)benzene-1,3-diamine (S-439)

S-438 (50 mg, 0.09 mmol) was mixed with 5% Pd/C (0.02 g, 0.01 mmol) in methanol (10 ml). The resulting mixture was stirred at room temperature overnight under hydrogen atmosphere. The reaction mixture was filtered and concentrated. The residue was purified by flash column chromatography (dichloromethane/methanol=15/1) to give the compound S-439 (17 mg, yield 35.1%). MS-ESI (M+H)⁺:492.22.

### Procedure for the Synthesis of 440

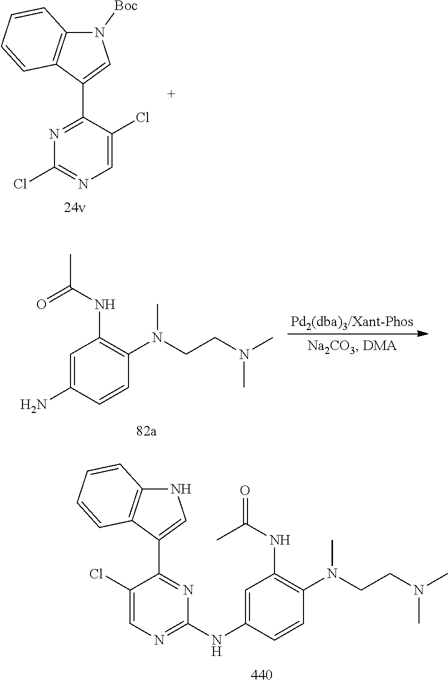 [SMILES: CC(C)(C)OC(=O)n1cc(-c2nc(Cl)ncc2Cl)c2ccccc21 ; CC(=O)Nc1cc(N)ccc1N(C)CCN(C)C ; CC(=O)Nc1cc(Nc2ncc(Cl)c(-c3c[nH]c4ccccc34)n2)ccc1N(C)CCN(C)C]

### N-(5-((5-chloro-4-(1H-indol-3-ylpyrimidin-2-yl) amino)-2-((2-(dimethylamino) ethyl)(methyl)amino)phenyl)acetamide (440)

To a solution of 24v (350 mg, 0.9615 mmol) in DMA (6 mL) was added 82a (240 mg, 0.96 mmol), Pd₂ (dba)₃ (88 mg, 0.096 mmol), Xant-Phos (84 mg, 0.144 mmol) and Na₂CO₃ (204 mg, 1.923 mmol). The mixture was stirred at 120° C. under Ar overnight. The mixture was cooled to room temperature, and filtered to give a solution. The solution was purified by Pre-HPLC (MeCN/0.05% HCOOH in water from 0 to 100%) to give the title product as formate, which was neutralized with saturated Na₂CO₃ to afford 440 (28.73 mg, yield 6.24%) as a yellow solid. MS-ESI (M+H)⁺: 478.2. ¹H NMR (400 MHz, DMSO-d₆) δ 11.88 (s, 1H), 10.00 (s, 1H), 9.52 (s, 1H), 8.61 (d, J=7.9 Hz, 1H), 8.52 (s, 1H), 8.41 (d, J=2.2 Hz, 1H), 8.40 (s, 1H), 7.53-7.46 (m, 2H), 7.20 (dd, J=12.7, 4.8 Hz, 2H), 7.09 (t, J=7.3 Hz, 1H), 2.81 (t, J=5.6 Hz, 2H), 2.66 (s, 3H), 2.30-2.26 (m, 2H), 2.22 (s, 6H), 2.05 (s, 3H).

### Procedure for the Synthesis of 441

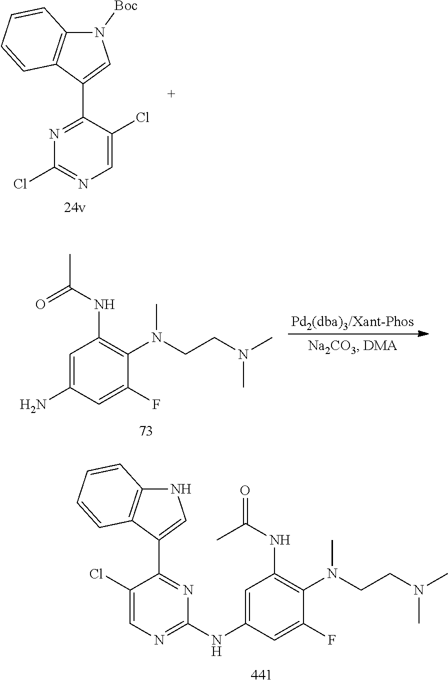 [SMILES: CC(C)(C)OC(=O)n1cc(-c2nc(Cl)ncc2Cl)c2ccccc21 ; CC(=O)Nc1cc(N)cc(F)c1N(C)CCN(C)C ; CC(=O)Nc1cc(Nc2ncc(Cl)c(-c3c[nH]c4ccccc34)n2)cc(F)c1N(C)CCN(C)C]

### N-(5-((5-chloro-4-(1H-indol-3-yl) pyrimidin-2-yl) amino)-2-((2-(dimethylamino) ethyl)(methyl)amino)-3-fluorophenyl)acetamide (441)

Synthesis of 441 was similar to that of 440. The solution was purified by Pre-HPLC (MeCN/0.05% HCOOH in water from 0 to 100%) to give the title product as formate, which was neutralized with saturated Na₂CO₃ to afford 441 (37.24 mg, yield 7.8%) as a yellow solid. MS-ESI (M+H): 496.2. ¹H NMR (400 MHz, DMSO-d₆) δ 10.39 (s, 1H), 9.80 (s, 1H), 8.66 (d, J=8.0 Hz, 1H), 8.57 (s, 1H), 8.45 (s, 1H), 8.23 (s, 1H), 7.66 (dd, J=14.9, 2.2 Hz, 1H), 7.54 (s, 1H), 7.23-7.18 (m, 1H), 7.15-7.10 (m, 1H), 2.98 (br, 2H), 2.75 (s, 3H), 2.22 (br, 8H), 2.07 (s, 3H).

### Procedure for the Synthesis of 442

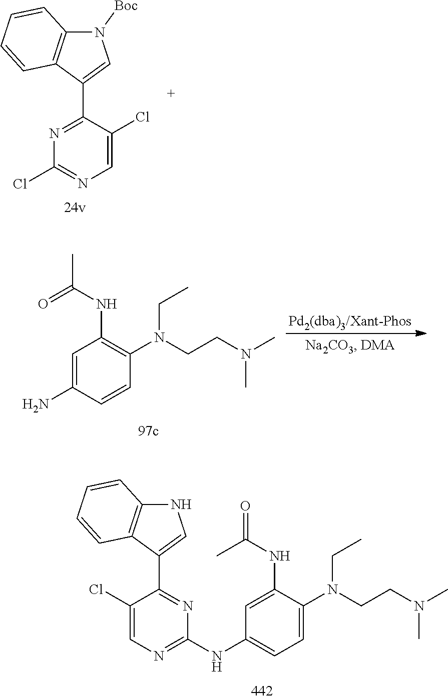 [SMILES: CC(C)(C)OC(=O)n1cc(-c2nc(Cl)ncc2Cl)c2ccccc21 ; CCN(CCN(C)C)c1ccc(N)cc1NC(C)=O ; CCN(CCN(C)C)c1ccc(Nc2ncc(Cl)c(-c3c[nH]c4ccccc34)n2)cc1NC(C)=O]

### N-(5-((5-chloro-4-(1H-indol-3-yl) pyrimidin-2-yl) amino)-2-((2-(dimethylamino) ethyl)(ethyl)amino)phenyl)acetamide (442)

Synthesis of 442 was similar to that of 440. The residue was purified by Pre-HPLC (MeCN/0.05% HCOOH in water from 0 to 100%) to give the title product as formate, which was neutralized with saturated Na₂CO₃ to afford 442 (29.1 mg, yield 4.3%) as a yellow solid. MS-ESI (M+H)⁺: 492.3. ¹H NMR (400 MHz, DMSO-d₆) δ 10.10 (s, 1H), 9.53 (s, 1H), 8.60 (d, J=8.0 Hz, 1H), 8.52 (s, 1H), 8.39 (s, 2H), 7.53 (dd, J=8.6, 2.5 Hz, 1H), 7.49 (d, J=7.9 Hz, 1H), 7.21 (d, J=8.6 Hz, 1H), 7.18 (d, J=7.2 Hz, 1H), 7.06 (t, J=7.5 Hz, 1H), 2.98-2.93 (m, 2H), 2.90 (t, J=5.7 Hz, 2H), 2.20-2.14 (m, 8H), 2.03 (s, 3H), 0.85 (t, J=7.1 Hz, 3H).

### Procedure for the Synthesis of 443

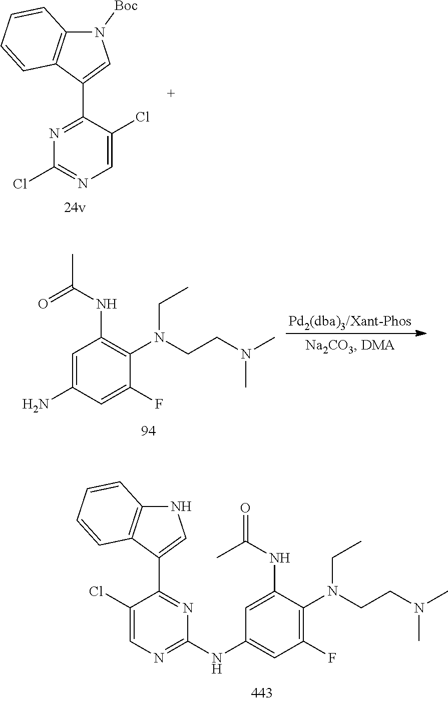 [SMILES: CC(C)(C)OC(=O)n1cc(-c2nc(Cl)ncc2Cl)c2ccccc21 ; CCN(CCN(C)C)c1c(F)cc(N)cc1NC(C)=O ; CCN(CCN(C)C)c1c(F)cc(Nc2ncc(Cl)c(-c3c[nH]c4ccccc34)n2)cc1NC(C)=O]

### N-(5-((5-chloro-4-(1H-indol-3-yl) pyrimidin-2-yl) amino)-2-((2-(dimethylamino) ethyl) (ethyl) amino)-3-fluorophenyl)acetamide (443)

Synthesis of 443 was similar to that of 440. The residue was purified by Pre-HPLC (MeCN/0.05% HCOOH in water from 0 to 100%) to give the title product as formate, which was neutralized with saturated Na₂CO₃ to afford 443 (80 mg, yield 11.4%) as a yellow solid. MS-ESI (M+H)⁺: 510.3. ¹H NMR (400 MHz, DMSO-d₆) δ 10.42 (s, 1H), 9.82 (s, 1H), 8.66 (d, J=7.9 Hz, 1H), 8.55 (d, J=2.0 Hz, 1H), 8.47 (s, 1H), 8.20 (s, 1H), 7.65 (dd, J=14.9, 2.4 Hz, 1H), 7.53 (d, J=8.3 Hz, 1H), 7.22 (t, J=7.5 Hz, 1H), 7.12 (t, J=7.4 Hz, 1H), 3.04 (d, J=7.0 Hz, 4H), 2.20 (d, J=8.7 Hz, 6H), 2.06 (s, 3H), 0.88 (t, J=7.1 Hz, 3H).

### Procedure for the Synthesis of 444

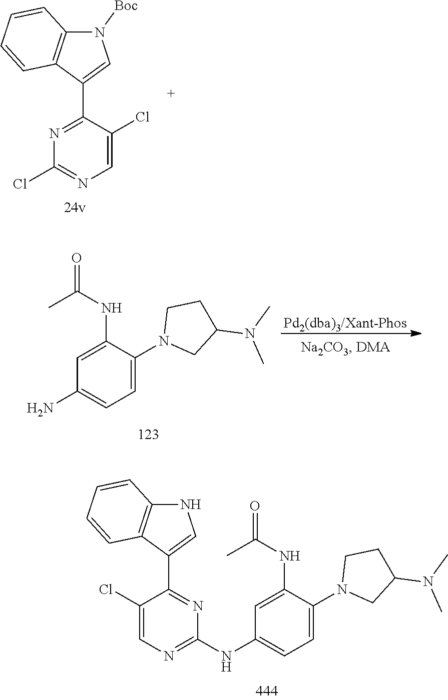 [SMILES: CC(C)(C)OC(=O)n1cc(-c2nc(Cl)ncc2Cl)c2ccccc21 ; CC(=O)Nc1cc(N)ccc1N1CCC(N(C)C)C1 ; CC(=O)Nc1cc(Nc2ncc(Cl)c(-c3c[nH]c4ccccc34)n2)ccc1N1CCC(N(C)C)C1]

### N-(5-((5-chloro-4-(1H-indol-3-yl) pyrimidin-2-yl) amino)-2-(3-(dimethylamino) pyrrolidin-1-yl) phenyl)acetamide (444)

Synthesis of 444 was similar to that of 440. The residue was purified by Pre-HPLC (MeCN/0.05% HCOOH in water from 0 to 100%) to give the title product as formate, which was neutralized with saturated Na₂CO₃ to afford 444 (28.6 mg, yield 4.2%) as a black solid. MS-ESI (M+H)⁺: 490.3. ¹H NMR (400 MHz, DMSO-d₆) δ 11.88 (s, 1H), 9.40 (s, 1H), 9.14 (s, 1H), 8.59 (d, J=7.6 Hz, 1H), 8.51 (s, 1H), 8.39 (s, 1H), 7.77 (s, 1H), 7.53-7.45 (m, 2H), 7.22 (t, J=7.1 Hz, 1H), 7.14 (t, J=7.3 Hz, 1H), 6.87 (d, J=8.7 Hz, 1H), 3.13-3.10 (m, 4H), 2.77-2.71 (m, 1H), 2.19 (s, 6H), 2.02 (s, 3H), 1.77-1.75 (m, 2H).

### Procedure for the Synthesis of 446

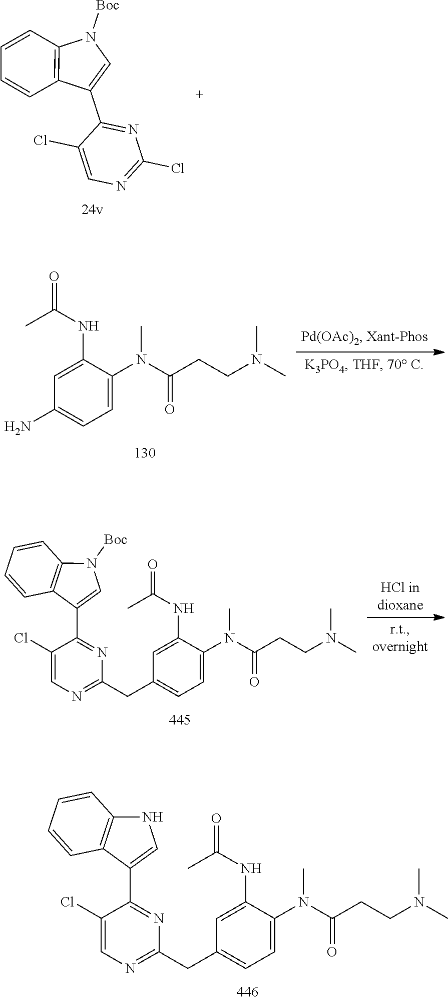

### tert-butyl 3-(2-((3-acetamido-4-(3-(dimethylamino)-N-methyl propanamido) phenyl) amino)-5-chloropyrimidin-4-yl)-1H-indole-1-carboxylate (445)

To a solution of 130 (300 mg, 1.08 mmol) in 15 mL of THF was added 24v (393 mg, 1.08 mmol), Pd(OAc)₂ (24 mg, 0.108 mmol), Xant-Phos (94 mg, 0.162 mmol) and K₃PO₄ (456 mg, 2.16 mmol). The mixture was stirred at 70° C. under Ar overnight. After cooling to room temperature, the mixture was filtered over Celite and washed with THF to give a solution. The solution was concentrated to give a residue. The residue was purified by flash column chromatography (MeCN/0.1% formic acid in water from 0 to 100%) to afford 445 (100 mg, yield 20%) as a yellow solid. MS-ESI (M+H): 606.3.

### N-(2-acetamido-4-((5-chloro-4-(1H-indol-3-yl)pyrimidin-2-yl) amino) phenyl)-3-(dimethylamino)-N-methylpropanamide (446)

A solution of 445 (120 mg, 0.198 mmol) in HCl (10 mL, 4M in dioxane) was stirred at rt overnight. The mixture was concentrated under reduced pressure to give a residue. The residue was purified by Pre-HPLC (MeCN/0.1% formic acid in water from 0 to 100%) to give the title product as formate, which was neutralized with saturated Na₂CO₃ to afford 446 (54.2 mg, yield 54.1%). MS-ESI (M+H): 506.3. ¹H NMR (400 MHz, CD₃OD) δ 8.58 (d, J=8.0 Hz, 1H), 8.45 (s, 1H), 8.37 (s, 1H), 8.33 (t, J=4.2 Hz, 1H), 7.66 (dd, J=8.6, 2.5 Hz, 1H), 7.46 (d, J=8.0 Hz, 1H), 7.22 (dd, J=14.7, 7.8 Hz, 2H), 7.15 (dd, J=11.1, 4.0 Hz, 1H), 3.17 (s, 3H), 2.73 (dt, J=12.5, 7.4 Hz, 1H), 2.54-2.47 (m, 1H), 2.33-2.27 (m, 2H), 2.13 (s, 6H), 2.10 (s, 3H).

### Procedure for the Syntheses of 448 and 449

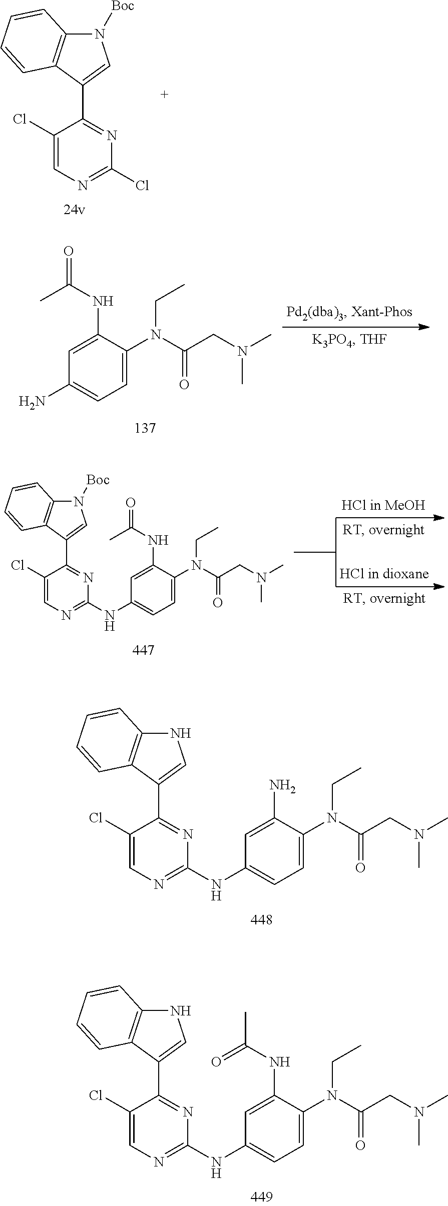 [SMILES: CC(C)(C)OC(=O)n1cc(-c2nc(Cl)ncc2Cl)c2ccccc21 ; CCN(C(=O)CN(C)C)c1ccc(N)cc1NC(C)=O ; CCN(C(=O)CN(C)C)c1ccc(Nc2ncc(Cl)c(-c3cn(C(=O)OC(C)(C)C)c4ccccc34)n2)cc1NC(C)=O ; CCN(C(=O)CN(C)C)c1ccc(Nc2ncc(Cl)c(-c3c[nH]c4ccccc34)n2)cc1N ; CCN(C(=O)CN(C)C)c1ccc(Nc2ncc(Cl)c(-c3c[nH]c4ccccc34)n2)cc1NC(C)=O]

### tert-butyl 3-(2-((3-acetamido-4-(2-(dimethylamino)-N-ethyla cetamido) phenyl) amino)-5-chloropyrimidin-4-yl)-1H-indole-1-carboxylate (447)

Synthesis of 447 was similar to that of 445. The residue was purified by flash column chromatography (MeCN/0.1% FA in water from 0 to 100%) to afford 447 (100 mg, yield 45%) as a yellow solid. MS-ESI (M+H)⁺: 606.3.

### N-(2-amino-4-((5-chloro-4-(1H-indol-3-yl)pyrimidin-2-yl)amino)phenyl)-2-(dimethylamino)-N-ethylacetamide (448)

A solution of 447 (100 mg, 0.165 mmol) in HCl (7 mL, in methanol) was stirred at rt overnight. The mixture was concentrated and the residue was purified by Pre-HPLC (MeCN/0.1% formic acid in water from 0 to 100%) to give the title product as formate, which was neutralized with saturated Na₂CO₃ to afford 448 (32 mg, yield 41.7%) as a yellow solid. MS-ESI (M+H)⁺: 464.3. ¹H NMR (400 MHz, CD₃OD) δ 8.50 (s, 1H), 8.33-8.27 (m, 2H), 7.42 (d, J=8.1 Hz, 1H), 7.35 (d, J=2.3 Hz, 1H), 7.18 (t, J=7.4 Hz, 1H), 7.05 (t, J=7.5 Hz, 1H), 6.97 (d, J=8.4 Hz, 1H), 6.64 (dd, J=8.4, 2.4 Hz, 1H), 3.92 (dd, J=13.5, 7.1 Hz, 1H), 3.40-3.36 (m, 1H), 2.96 (d, J=15.2 Hz, 1H), 2.82 (d, J=15.2 Hz, 1H), 1.98 (s, 6H), 1.10 (t, J=7.1 Hz, 3H).

### N-(2-acetamido-4-((5-chloro-4-(1H-indol-3-yl)pyrimidin-2-yl)amino)phenyl)-2-(dimethylamino)-N-ethylacetamide (449)

A solution of 447 (120 mg, 0.198 mmol) in HCl (10 mL, 4 M in dioxane) was stirred at rt overnight. The mixture was concentrated and the residue was purified by Pre-HPLC (MeCN/0.1% formic acid in water from 0 to 100%) to give the title product as formate, which was neutralized with saturated Na₂CO₃ to afford 449 (77 mg, yield 77%) as a white solid. MS-ESI (M+H)⁺: 506.3. ¹H NMR (400 MHz, CD₃OD) δ 8.52 (s, 1H), 8.31 (s, 1H), 8.18 (t, J=5.8 Hz, 2H), 7.67 (dd, J=8.6, 2.4 Hz, 1H), 7.42 (d, J=8.1 Hz, 1H), 7.25 (d, J=8.6 Hz, 1H), 7.16 (t, J=7.2 Hz, 1H), 6.94 (t, J=7.5 Hz, 1H), 3.96 (dd, J=13.6, 7.1 Hz, 1H), 3.42 (dd, J=13.6, 7.1 Hz, 1H), 2.93 (d, J=15.2 Hz, 1H), 2.82 (d, J=15.2 Hz, 1H), 2.07 (s, 3H), 1.99 (s, 6H), 1.11 (t, J=7.2 Hz, 3H).

### Procedure for the Synthesis of 451

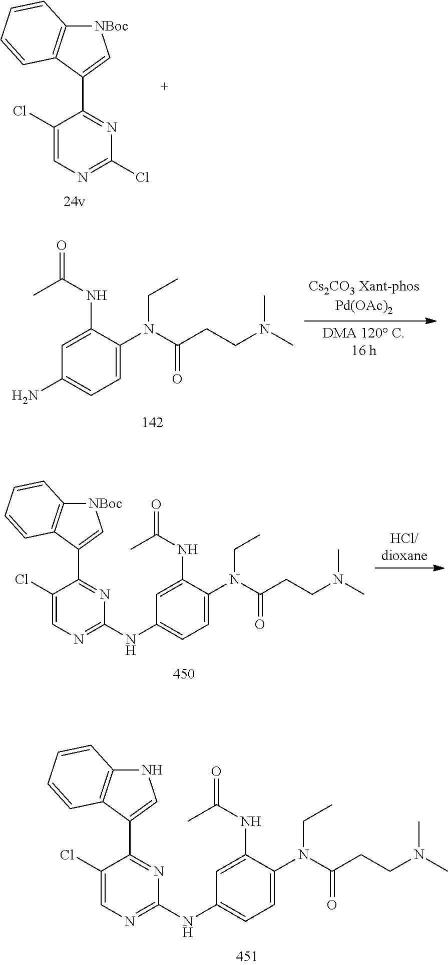 [SMILES: CC(C)(C)OC(=O)n1cc(-c2nc(Cl)ncc2Cl)c2ccccc21 ; CCN(C(=O)CCN(C)C)c1ccc(N)cc1NC(C)=O ; CCN(C(=O)CCN(C)C)c1ccc(Nc2ncc(Cl)c(-c3cn(C(=O)OC(C)(C)C)c4ccccc34)n2)cc1NC(C)=O ; CCN(C(=O)CCN(C)C)c1ccc(Nc2ncc(Cl)c(-c3c[nH]c4ccccc34)n2)cc1NC(C)=O]

### tert-butyl3-(2-((3-acetamido-4-(3-(dimethylamino)-N-ethylpropanamido)phenyl)amino)-5-chloropyrimidin-4-yl)-1H-indole-1-carboxylate (450)

A mixture of 142 (292.0 mg, 1.0 mmol), K₃PO₄ (422.0 mg, 2.0 mmol), 24v (360 mg, 1.0 mmol), Xant-phos (87.0 mg, 0.15 mmol) and Pd(OAc)₂ (25 mg, 0.1 mmol) in THF (20 mL) was stirred at 80° C. under Ar overnight. After cooling to room temperature, the mixture was filtered and the filtrate was concentrated and purified by flash column chromatography (methanol/dichloromethane=10/1) to give 450 as a white solid (200 mg, yield 32.0%). MS-ESI (M+H)⁺: 620.2.

### N-(2-acetamido-4-((5-chloro-4-(1H-indol-3-yl)pyrimidin-2-yl)amino)phenyl)-3-(dimethylamino)-N-ethylpropanamide (451)

A solution of 450 (200 mg, 0.32 mmol) in 4M HCl/dixoane (10 mL) was stirred for 1 h at room temperature. The mixture was concentrated and the residue was purified by pre-HPLC (MeCN/0.05% NH₃ in water from 0 to 100%)) to give the title product 451 as a white solid (95.9 mg, yield 47%). MS-ESI (M+H)⁺: 520.2. ¹H NMR (400 MHz, DMSO-d₆) δ 11.93 (s, 1H), 9.81 (s, 1H), 9.28 (s, 1H), 8.62 (d, J=7.9 Hz, 1H), 8.54-8.47 (m, 2H), 8.18 (s, 1H), 7.71 (dd, J=8.6, 2.2 Hz, 1H), 7.50 (d, J=8.0 Hz, 1H), 7.22 (t, J=7.5 Hz, 1H), 7.14 (dd, J=11.9, 6.9 Hz, 2H), 3.94 (dd, J=13.6, 7.1 Hz, 1H), 3.06 (dd, J=13.7, 7.0 Hz, 1H), 2.47-2.43 (m, 2H), 2.13-2.08 (m, 2H), 2.04 (s, 3H), 2.01 (s, 6H), 0.98 (t, J=7.1 Hz, 3H).

### Procedure for the Synthesis of 452

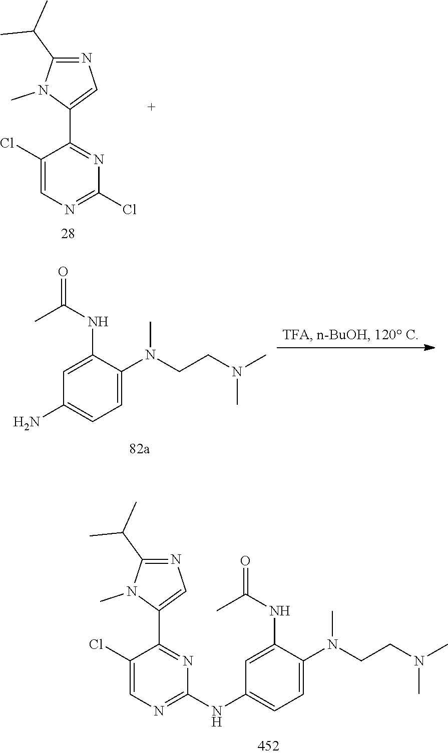 [SMILES: CC(C)c1ncc(-c2nc(Cl)ncc2Cl)n1C ; CC(=O)Nc1cc(N)ccc1N(C)CCN(C)C ; CC(=O)Nc1cc(Nc2ncc(Cl)c(-c3cnc(C(C)C)n3C)n2)ccc1N(C)CCN(C)C]

### N-(5-((5-chloro-4-(2-isopropyl-1-methyl-1H-imidazol-5-yl)pyrimidin-2-yl)amino)-2-((2-(dimethylamino)ethyl)(methyl)amino)phenyl)acetamide (452)

To the mixture of 28 (85 mg, 0.31 mmol) and 82a (78 mg, 0.31 mmol) in n-BuOH (4 mL) was added TFA (0.05 mL, 0.63 mmol). The mixture was stirred at 120° C. under Ar for 16 h. The mixture was concentrated in vacuo and the residue was purified by Prep-HPLC (NH₃H₂O) to give 452 (32 mg, 0.066 mmol, 21.05% yield) as white solid. MS-ESI (M+H)⁺: 485.2; ¹H NMR (400 MHz, DMSO-d₆) δ 10.09 (s, 1H), 9.97 (s, 1H), 8.28 (s, 1H), 7.52-7.33 (m, 2H), 7.29 (d, J=8.7 Hz, 1H), 6.95 (s, 1H), 3.84 (s, 3H), 3.15 (dt, J=13.7, 6.8 Hz, 1H), 2.79 (t, J=5.2 Hz, 2H), 2.66 (s, 3H), 2.35-2.27 (m, 2H), 2.23 (s, 6H), 2.09 (s, 3H), 1.24 (d, J=6.8 Hz, 6H).

### Synthesis of Compounds of Formula IV

### Experimental Section for the Syntheses of Aryl Amines

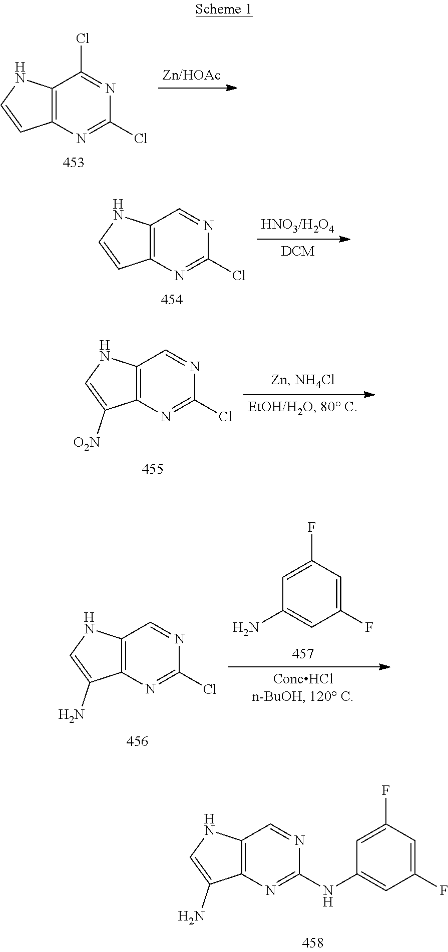 [SMILES: Clc1nc(Cl)c2[nH]ccc2n1 ; Clc1ncc2[nH]ccc2n1 ; O=[N+]([O-])c1c[nH]c2cnc(Cl)nc12 ; Nc1c[nH]c2cnc(Cl)nc12 ; Nc1cc(F)cc(F)c1 ; Nc1c[nH]c2cnc(Nc3cc(F)cc(F)c3)nc12]

### 2-chloro-5H-pyrrolo[3,2-d]pyrimidine (454)

To a solution of 2,4-dichloro-5H-pyrrolo[3,2-d]pyrimidine 453 (15 g, 80.25 mmol) in MeOH (300 mL) was added CH₃COOH (39 mL) and Zn (26.1 g, 400 mmol). The mixture was stirred at 80° C. for 12 h. The mixture was filtered and the filtrate was concentrated. The residue was extracted with dichloromethane/methanol=10/1. The combined organic layers were concentrated to give the desired product 454 (10.2 g, yield 83.6%) as a yellow solid. ¹H NMR (400 MHz, DMSO-d₆) δ 12.08 (s, 1H), 8.84 (s, 1H), 8.03 (d, J=2.8 Hz, 1H), 6.61 (d, J=2.8 Hz, 1H).

### 2-chloro-7-nitro-5H-pyrrolo[3,2-d]pyrimidine (455)

A mixture of fuming nitric acid (17 mL) and concentrated sulfuric acid (17 mL) was prepared by dropwise addition of H₂SO₄ to ice-cooled and stirred HNO₃, and immediately added dropwise to the solution of 454 (9 g, 0.059 mmol) in dichloromethane over 10 min. After stirring for another 1 h, the reaction was quenched by addition of chilled 20% aqueous K₂CO₃ to adjusted the pH=7. The mixture was concentrated, the residue was extracted with dichloromethane/methanol=10/1. The organic phase was concentrated to give the desired product. 455 (10.5 g. yield 90.23%). ¹H NMR (400 MHz, DMSO-d₆) δ 8.59 (s, 1H), 8.41 (s, 1H).

### 2-chloro-5H-pyrrolo[3,2-d]pyrimidin-7-amine (456)

To the mixture of 455 (5 g, 25.18 mmol) in EtOH (120 mL) and H₂O (20 mL) were added Zn powder (9.88 g, 151.08 mmol) and NH₄Cl (16.16 g, 302.17 mmol). The mixture was stirred at 80° C. under Ar for 3 h. The mixture was concentrated under reduced pressure. The residue was washed with EtOH (70 mL*5). The organic phase was concentrated in vacuo. The residue was purified by flash column chromatography (dichloromethane/methanol=20/1-10/1) to give 456 (1.9 g, yield 45%) as black solid. MS-ESI (M+H)⁺: 169.1.

### N²-(3,5-difluorophenyl)-5H-pyrrolo[3,2-d]pyrimidine-2,7-diamine (458)

To the mixture of 456 (1.7 g, 11.07 mmol) and 3,5-difluoroaniline 457 (2.14 g, 16.60 mmol) in n-BuOH (25 mL) was added conc. HCl (5.07 mL, 60.88 mmol). The mixture was stirred at 120° C. under Ar for 16 h. The mixture was concentrated in vacuo. The residue was dissolved in dichloromethane/methanol (10/1, 100 mL) and H₂O (30 mL). Sat. NaHCO₃ (20 mL) was added to adjust the pH=8. The mixture was extracted with dichloromethane/methanol (10/1, 50 mL*3). The organic phase was dried over Na₂SO₄, filtered and concentrated. The residue was purified by flash column chromatography (dichloromethane/methanol=20/1-10/1) to give 458 (250 mg, 0.96 mmol, yield 9.49%) as yellow solid. MS-ESI (M+H)⁺: 262.0. ¹H NMR (400 MHz, DMSO) δ 10.69 (s, 1H), 9.58 (s, 1H), 8.55 (s, 1H), 7.67 (d, J=9.6 Hz, 2H), 7.11 (d, J=1.8 Hz, 1H), 6.56 (t, J=9.2 Hz, 1H), 4.12 (s, 2H).

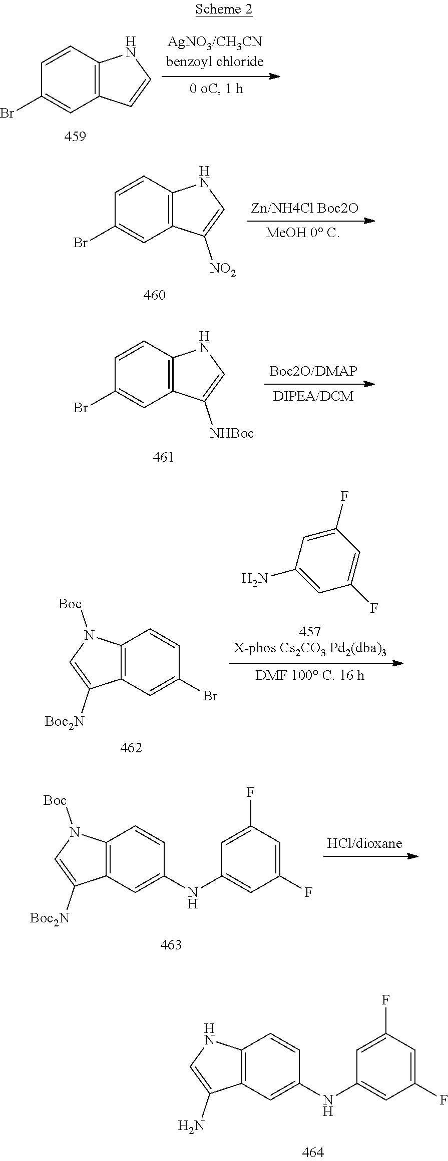

### 5-bromo-3-nitro-1H-indole (460)

To a mixture of 459 (10.0 g, 51.0 mmol) and AgNO₃ (10.4 g, 61.2 mmol) in CH₃CN (150 mL) was added benzoyl chloride (7.1 mL, 61.2 mmol) at 0° C. and the mixture was stirred for this temperature for 1 h. The mixture was concentrated in vacuo. The residue was purified by flash column chromatography (petroleum ether/ethyl acetate=5/1) to provide target compound 460 (10.5 g, 83.7% yield) as a brown solid. ¹H NMR (400 MHz, CD₃OD) δ 8.38 (s, 1H), 8.29 (t, J=1.3 Hz, 1H), 7.45 (d, J=1.2 Hz, 2H).

### tert-butyl (5-bromo-1H-indol-3-yl)carbamate (461)

A mixture of 460 (7 g, 29.0 mmol) and NH₄Cl (15.0 g, 290 mmol) in MeOH (150 mL) and water (30 mL) was stirred for 10 min at 0° C., then (Boc)₂O (9.40 g, 43.5 mmol) and Zn (18.8 g, 290 mmol) was added. The mixture was stirred for 2 h at this temperature. After filtration, the filtrate was concentrated and the residue was purified by flash column chromatography (petroleum ether/ethyl acetate=10/1) to give target compound 461 (5.2 g, 57.8% yield). MS-ESI (M+H)⁺: 254.9, 312.9.

### tert-butyl-5-bromo-3-(di(tert-butoxycarbonyl)amino)-1H-indole-1-carboxylate (462)

A solution of 461 (5 g, 16.1 mmol), DIEA (4.9 g, 48.3 mmol), DMAP (200 mg, 1.6 mmol) and (Boc)2O (10.5 g, 48.3 mmol) in dichloromethane (100 mL) was stirred at room temperature for 2 h. After concentration, the residue was purified by flash column chromatography (petroleum ether/ethyl acetate=10/1) to give the title product 462 as a white solid (6 g, 72.7% yield). ¹H NMR (400 MHz, CDCl₃) δ 8.02 (d, J=6.9 Hz, 1H), 7.56-7.49 (m, 2H), 7.42-7.37 (dd, J=8.8, 1.9 Hz, 1H), 1.66 (s, 9H), 1.46 (s, 18H).

### tert-butyl 3-(di(tert-butoxycarbonyl)amino)-5-((3,5-difluorophenyl)amino)-1H-indole-1-carboxylate (463)

A mixture of 462 (3.0 g, 5.9 mmol), 3,5-difluoroaniline 457 (820 mg, 6.5 mmol), X-Phos (230 mg, 0.48 mmol), Pd(OAc)₂ (110 mg, 0.48 mmol) and Cs₂CO₃ (3.8 g, 11.8 mmol) in toluene (100 mL) was stirred for 16 h at 120° C. under nitrogen. After cooling to room temperature, the mixture was filtered and the filtrate was concentrated. The residue was purified by flash column chromatography (petroleum ether/ethyl acetate=5/1) to give the title product 463 as a white solid (1.8 g, 54.2% yield). ¹H NMR (400 MHz, CDCl₃) δ 8.10 (s, 1H), 7.56 (s, 1H), 7.15 (s, 1H), 7.09 (d, J=6.9 Hz, 1H), 6.42-6.35 (m, 2H), 6.26-6.20 (m, 1H), 5.85 (s, 1H), 1.67 (s, 9H), 1.45 (s, 18H)

### N⁵-(3,5-difluorophenyl)-1H-indole-3,5-diamine Hydrochloride (464)

A solution of 463 (1.8 g, 3.2 mmol) in 4 M HCl in 1,4-dioxane (20 mL) was stirred for 2 h at room temperature. After concentration, the crude title product 464 was obtained as a brown solid, which was used to next step without further purification.

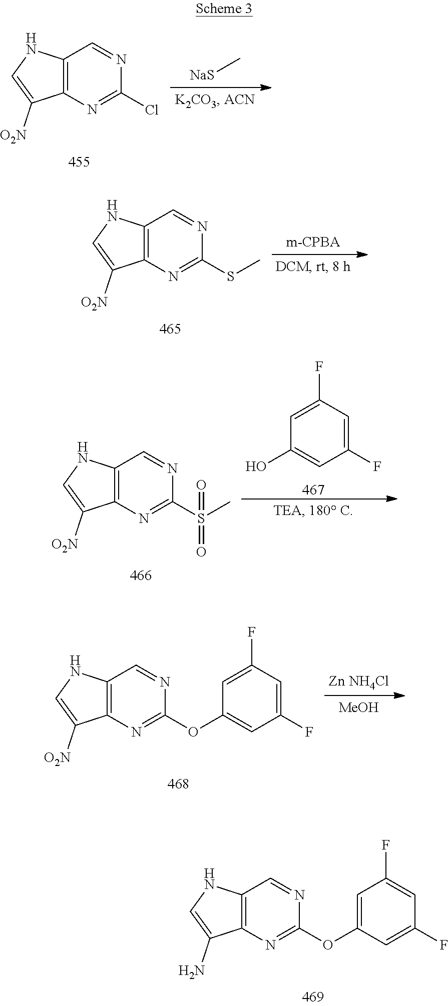

### 2-(methylthio)-7-nitro-5H-pyrrolo[3,2-d]pyrimidine (465)

To the mixture of 455 (8 g, 40.29 mmol) in CH₃CN (200 mL) were added sodium methanethiolate (5.65 g, 80.58 mmol) and K₂CO₃ (11.14 g, 80.58 mmol). The mixture was stirred at 80° C. for 8 h. The mixture was filtered, the filtrate was concentrated and the residue was purified by flash column chromatography (dichloromethane/methanol=100/1-10/1) to give 465 (7.0 g, 82.65% yield) as a yellow solid. MS-ESI (M+H)⁺:210.9; ¹H NMR (400 MHz, CDCl₃) δ 8.59 (s, 1H), 8.30 (s, 1H), 2.54 (s, 3H).

### 2-(methylsulfonyl)-7-nitro-5H-pyrrolo[3,2-d]pyrimidine (466)

To the mixture of 465 (7 g, 33.3 mmol) in dichloromethane (200 mL) was added m-CPBA (13.52 g, 66.60 mmol). The mixture was stirred at 20° C. for 8 h. The mixture was quenched with saturated NaHSO₃ solution and extracted with dichloromethane/methanol=10/1 (500 mL*3). The combined organic phase was concentrated and purified by flash column chromatography (dichloromethane/methanol=100/1-10/1) to give the 466 (4.5 g, 55.79% yield) as pale yellow solid. MS-ESI (M+H)⁺: 243.0; ¹H NMR (400 MHz, CDCl₃) δ 9.13 (s, 1H), 8.72 (s, 1H), 3.28-3.17 (s, 3H).

### 2-(3,5-difluorophenoxy)-7-nitro-5H-pyrrolo[3,2-d]pyrimidine (468)

To the mixture of 466 (2.0 g, 8.26 mmol) in TEA (5 mL) were added 3,5-difluorophenol 467 (2.15 g, 16.51 mmol). The mixture was stirred at 140° C. for 8 h. The mixture was purified by flash column chromatography (dichloromethane/methanol=100/1˜10/1) to give 468 (200 mg, 8.29% yield) as a brown solid. MS-ESI (M+H)⁺: 293.0. ¹H NMR (400 MHz, CDCl₃) δ 8.67 (s, 1H), 8.43 (s, 1H), 6.76 (s, 2H), 6.62 (s, 1H).

### 2-(3,5-difluorophenoxy)-5H-pyrrolo[3,2-d]pyrimidin-7-amine (469)

To a solution of 468 (200 mg, 0.684 mmol) in MeOH (30 mL) was added Zn (447.50 mg, 6.84 mmol) and NH₄Cl (732.22 mg, 13.69 mmol). The mixture was stirred at 20° C. for 8 h. The mixture was filtered and the filtrate was concentrated. The residue was dissolved in dichloromethane/methanol (10/1, 200 mL), washed with saturated NaCl solution. The organic phase was dried over Na₂SO₄, filtered. The filtrate was concentrated to give the 469 (80 mg, crude) as a brown solid, which was used to next step without further purification. MS-ESI (M+H)⁺:263.2.

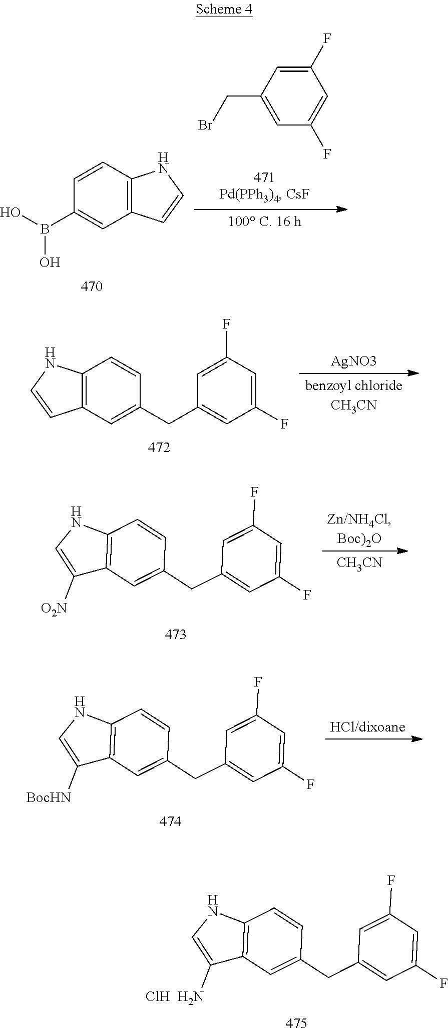

### 5-(3,5-difluorobenzyl)-1H-indole (472)

A mixture of 470 (9.0 g, 56.1 mmol), CsF (26.4 g, 173.6 mmol), 471 (9.0 g, 43.4 mmol) and Pd(PPh₃)₄ (3.0 g, 2.6 mmol) in 1,2-dimethoxyethane (100 mL) was stirred at 100° C. under Ar overnight. After cooling to room temperature, the mixture was concentrated, the residue was purified by flash column chromatography (petroleum ether/ethyl acetate=2/1) to give the title product 472 as a white solid (6.7 g, yield 49%). MS-ESI (M+H)⁺: 244.2. ¹H NMR (400 MHz, DMSO-d₆) δ 11.02 (br s, 1H), 7.42 (s, 1H), 7.32 (d, J=8.4 Hz, 2H), 7.05-6.95 (m, 4H), 6.37 (s, 1H), 4.01 (s, 2H).

### 5-(3,5-difluorobenzyl)-3-nitro-1H-indole (473)

A mixture of 472 (3.3 g, 13.6 mmol) and AgNO₃ (2.7 g, 15.6 mmol) in CH₃CN (30 mL) was stirred at 0° C. for 10 min. benzoyl chloride (2.3 g, 16.3 mmol) was added and the mixture was stirred at 0° C. for another 1 h. The mixture was filtered and the filtrate was concentrated. The residue was purified by reverse phase column chromatography (MeCN/0.05% NH₃ in water from 0 to 100%) to give the title product 473 as a brown solid (1.1 g, yield 21%). MS-ESI (M−H)⁻: 287.2. ¹H NMR (400 MHz, DMSO-d₆) δ 12.61 (br s, 1H), 8.63 (s, 1H), 8.00 (s, 1H), 7.50 (d, J=4.4 Hz, 1H), 7.25 (d, J=4.8 Hz, 1H), 7.10-6.95 (m, 3H), 4.13 (s, 2H).

### tert-butyl (5-(3,5-difluorobenzyl)-1H-indol-3-yl)carbamate (474)

To a mixture of 473 (1.1 g, 3.6 mmol) and NH₄Cl (1.9 g, 36.0 mmol) in (MeOH/H₂O=100/10 mL) was added Zn (2.5 g, 36.0 mmol) at 0° C. After 10 min, (Boc₂)O was added and the mixture was stirred at 0° C. for 1 h. The mixture was filtered. The filtrate was concentrated and the residue was purified by reverse phase column chromatography (MeCN/0.05% NH₃ in water from 0 to 100%) to give the title product 474 as a brown solid (400 mg, yield 30.1%). ¹H NMR (400 MHz, DMSO-d₆) δ 10.65 (s, 1H), 8.98 (s, 1H), 7.60 (s, 1H), 7.36 (s, 1H), 7.22 (d, J=8.2 Hz, 1H), 6.99-6.95 (m, 4H), 3.99 (s, 2H), 1.48 (s, 9H).

### 5-(3,5-difluorobenzyl)-1H-indol-3-amine hydrochloride (475)

A solution of 474 (280 mg, 0.79 mmol) in 4 M HCl/dixoane (10 mL) was stirred at room temperature for 1 h. The mixture was concentrated and the crude product 475 was used in next step without further purification.

### Methods for the Syntheses of Aryl Formic Acid

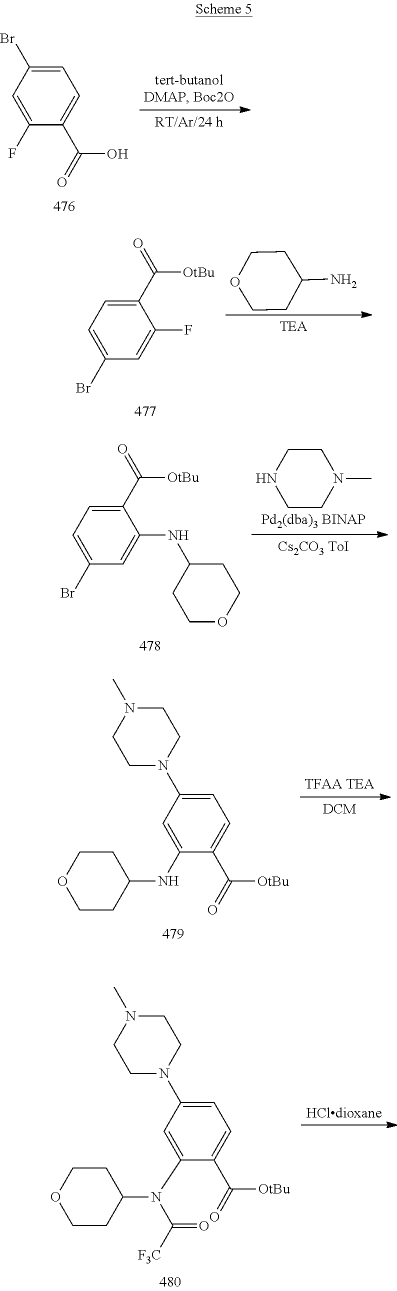

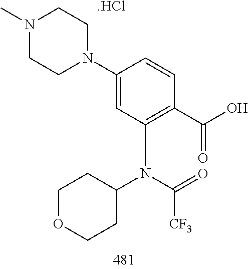 [SMILES: ".HCl" ; CN1CCN(c2ccc(C(=O)O)c(N(C(=O)C(F)(F)F)C3CCOCC3)c2)CC1]

### tert-butyl 4-bromo-2-fluorobenzoate (477)

A mixture of 4-bromo-2-fluorobenzoic acid 476 (10.0 g, 45.6 mmol), (Boc)₂O (21.0 g, 91.2 mmol) and DMAP (2.6 g, 21.7 mmol) in dichloromethane (150 mL) and tert-butanol (45 mL) was stirred at room temperature overnight. After concentration, the residue was purified by flash column chromatography (petroleum ether/ethyl acetate=10/1) to give 477 (10.5 g, yield 83.7%) as a colorless oil. ¹H NMR (400 MHz, CDCl₃) δ 7.64 (t, J=8.0 Hz, 1H), 7.25-7.13 (m, 2H), 1.49 (s, 9H).

### tert-butyl 4-bromo-2-((tetrahydro-2H-pyran-4-yl)amino)benzoate (478)

A mixture of 477 (3.7 g, 13.3 mmol), tetrahydro-2H-pyran-4-amine (2.0 g, 20.0 mmol) and TEA (3.6 g, 26.7 mmol) was stirred at 100° C. overnight in a sealed tube. After cooling to room temperature, the mixture was diluted with ethyl acetate (200 mL). The organic phase was washed with H₂O (250 mL) and NaCl (250 mL*2), dried with Na₂SO₄. After concentration, the residue was purified by flash column chromatography (petroleum ether/ethyl acetate=10:1) to provide 478 (2.9 g, 61.2% yield). ¹H NMR (400 MHz, DMSO-d₆) δ 7.76 (d, J=7.6 Hz, 1H), 7.66 (d, J=8.4 Hz, 1H), 7.03 (s, 1H), 6.72 (d, J=8.4 Hz, 1H), 3.84 (d, J=11.6 Hz, 2H), 3.76-3.66 (m, 1H), 3.49 (t, J=11.1 Hz, 2H), 1.92 (d, J=12.2 Hz, 2H), 1.53 (s, 9H), 1.45-1.33 (m, 2H).

### tert-butyl4-(4-methylpiperazin-1-yl)-2-((tetrahydro-2H-pyran-4-yl)amino)benzoate (479)

To a mixture of 478 (900 mg, 2.53 mmol) and 1-methylpiperazine (1.0 g, 10.11 mmol) in toluene (20 mL) were added Cs₂CO₃ (1.6 g, 2.1 mmol), Pd₂(dba)₃ (120 mg, 1.3 mmol) and BINAP (156 mg, 0.25 mmol). The mixture was stirred at 100° C. under Ar overnight. After cooling to room temperature, the mixture was diluted with ethyl acetate (200 mL). The organic phase was washed with H₂O (250 mL) and NaCl (250 mL*2), dried with Na₂SO₄. After concentration, the residue was purified by flash column chromatography (dichloromethane/methanol=10/1) to give 479 as a white solid (850 mg, yield 89.6%). MS-ESI (M+H)⁺: 376.2.

### N-(3-((5-chloro-4-((2-methoxy-4-(piperazin-1-yl)phenyl)amino)pyrimidin-2-yl)amino)phenyl)propionamide (480)

Under nitrogen atmosphere 479 (1.1 g, 2.9 mmol) was dissolved in dichloromethane (10 ml) and cooled to 0° C. Then TEA (0.8 mL, 5.8 mmol) was added followed by a slow addition of TFAA (0.5 mL, 3.9 mmol). Reaction was terminated after 4 hours and diluted with dichloromethane. The mixture was washed with a saturated solution of aqueous NaHCO₃ (100 mL), brine (100 mL) and dried over sodium sulfate, filtered and evaporated to dryness affording the title compound 480 as an orange foam (650 mg, 47.8%). MS-ESI (M+H)⁺: 472.3

### 4-(4-methylpiperazin-1-yl)-2-(2,2,2-trifluoro-N-(tetrahydro-2H-pyran-4-yl)acetamido)benzoic acid (481)

To the solution of 480 (110 mg, 0.23 mmol) in 1,4-dioxane (1.5 mL) was added conc. HCl (0.5 mL, 6.00 mmol). The reaction mixture was stirred at 25° C. for 6 h. The mixture was concentrated to give 481 (95 mg, yield 98%) as colorless oil, which was used in the next step without further purification. MS-ESI (M+H)⁺: 416.1.

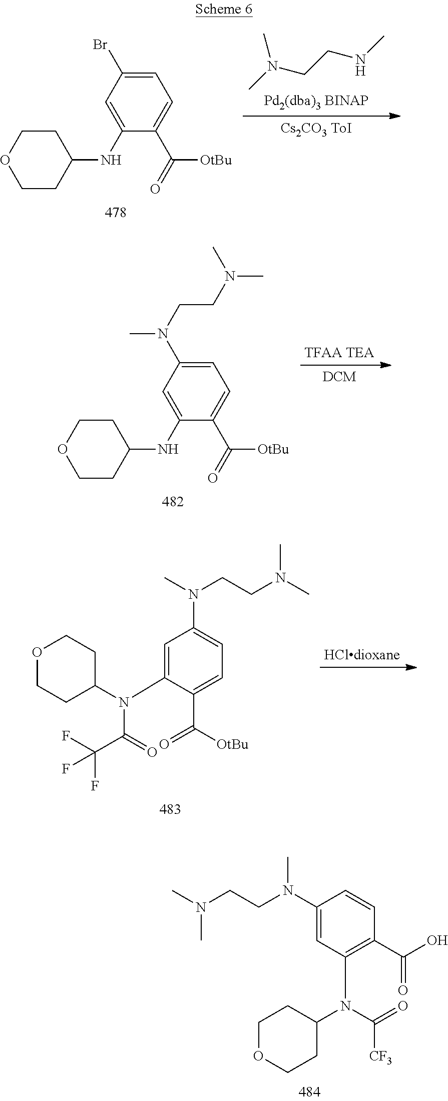

### tert-butyl4-((2-(dimethylamino)ethyl)(methyl)amino)-2-((tetrahydro-2H-pyran-4-yl)amino)benzoate (482)

To a solution of 478 (1.5 g, 4.2 mmol) and N¹,N¹,N²-trimethylethane-1,2-diamine (1.7 g, 8.4 mmol) in toluene (30 mL) were added Cs₂CO₃ (5.5 g, 8.4 mmol), Pd₂(dba)₃ (290 mg, 0.2 mmol) and BINAP (510 mg, 0.14 mmol). The mixture was stirred at 100° C. under Ar overnight. After cooling to room temperature, the mixture was diluted with ethyl acetate (200 mL). The organic phase was washed with H₂O (250 mL) and NaCl (250 mL*2), dried with Na₂SO₄. After concentration, the residue was purified by flash column chromatography (dichloromethane/methanol=20/1) to give the title product 482 as a white solid (1.4 g, 88.6%). MS-ESI (M+H)⁺: 378.2.

### tert-butyl4-((2-(dimethylamino)ethyl)(methyl)amino)-2-(2,2,2-trifluoro-N-(tetrahydro-2H-pyran-4-yl)acetamido)benzoate (483)

Under nitrogen atmosphere 482 (350 mg, 0.93 mmol) was dissolved in dichloromethane (10 ml) and cooled to 0° C. Then TEA (0.28 mL, 1.86 mmol) was added followed by a slow addition of TFAA (0.2 mL, 1.2 mmol). The mixture was stirred at room temperature for 4 h. After diluting with water, the mixture was extracted with dichloromethane, washed with a saturated solution of aqueous NaHCO₃ (100 mL), brine solution (100 mL), dried over sodium sulfate, filtered and evaporated to dryness affording the title compound 483 as an orange foam (360 mg, yield 81.8%). MS-ESI (M+H)⁺: 474.6.

### 4-((2-(dimethylamino)ethyl)(methyl)amino)-2-(2,2,2-trifluoro-N-(tetrahydro-2H-pyran-4-yl)acetamido)benzoic acid (484)

A solution of 483 (200 mg, 0.42 mmol) in HCl-dioxane (4 mL) was stirred at 25° C. for 8 h. The mixture was concentrated in vacuo (oil pump, room temperature) to give 484 (176 mg, ˜100% yield) as colorless oil, which was used in the next step without further purification. MS-ESI (M+H)⁺: 418.5.

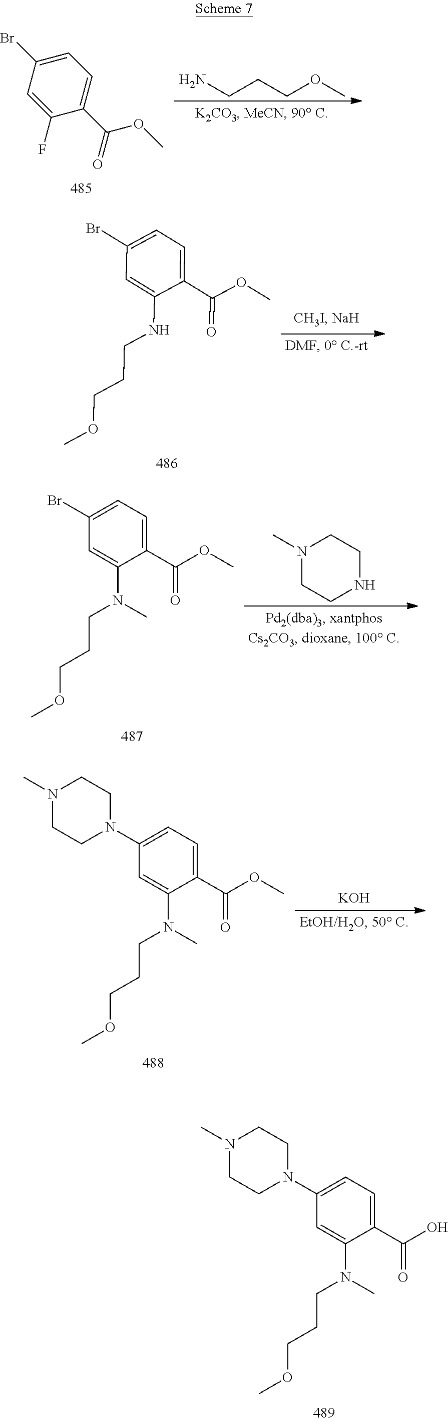

### methyl 4-bromo-2-((3-methoxypropyl)amino)benzoate (486)

To the mixture of 485 (10 g, 42.91 mmol) and 3-methoxypropan-1-amine (8.76 mL, 85.82 mmol) in MeCN (100 mL) was added K₂CO₃ (11.86 g, 85.82 mmol). The mixture was stirred at 90° C. under Ar for 16 h. The reaction mixture was concentrated in vacuo. The residue was dissolved with water (50 mL) and extracted with ethyl acetate (80 mL*3). The organic phase was dried by Na₂SO₄, filtered and concentrated. The residue was purified by flash column chromatography (SiO₂, petroleum ether/ethyl acetate=20/1-10/1) to give 486 (11.0 g, 36.40 mmol, 84.83% yield) as colorless oil. MS-ESI (M+H⁺+2): 303.9.

### methyl 4-bromo-2-((3-methoxypropyl)(methyl)amino)benzoate (487)

To the mixture of NaH (1.32 g, 33.09 mmol) in DMF (30 mL) was added 486 (5 g, 16.55 mmol) in DMF (20 mL) dropwise at 0° C. under Ar. The mixture was stirred at 0° C. under Ar for 1 h. CH₃I (2.06 mL, 33.09 mmol) was added dropwise to the mixture at 0° C. The mixture was stirred at 25° C. under Ar for 40 h. The reaction was quenched with sat.NH₄Cl (20 mL) and concentrated in vacuo. The residue was dissolved with water (20 mL) and extracted with ethyl acetate (60 mL*3). The organic phase was washed with brine (50 mL*3), dried by Na₂SO₄, filtered and concentrated. The residue was purified by flash column chromatography (SiO₂, petroleum ether/ethyl acetate=20/1-10/1-5/1) to give 487 (4.84 g, 92.51% yield) as colorless oil. MS-ESI (M+H⁺+2): 317.9.

### methyl 2-((3-methoxypropyl)(methyl)amino)-4-(4-methylpiperazin-1-yl)benzoate (488)

To the mixture of 487 (4.8 g, 15.18 mmol) and 1-methylpiperazine (3.37 mL, 30.36 mmol) in dioxane (60 mL) were added Pd₂(dba)₃ (695 mg, 0.76 mmol), XantPhos (878 mg, 1.52 mmol) and Cs₂CO₃ (9.89 g, 30.36 mmol). The mixture was stirred at 100° C. under Ar for 16 h. After concentration, the residue was dissolved with water (80 mL). The aqueous phase was acidified with conc. HCl to pH=1 at 0° C. and extracted with ethyl acetate (50 mL*3). The aqueous phase was basified with NaOH to pH=9 at 0° C. and extracted with dichloromethane/methanol (10/1, 50 mL*5). The organic phase was dried by Na₂SO₄, filtered and concentrated in vacuo to give 488 (5.7 g, ˜100% yield), which was used in the next step without further purification. MS-ESI (M+H⁺): 336.3.

### 2-((3-methoxypropyl)(methyl)amino)-4-(4-methylpiperazin-1-yl)benzoic acid (489)

To the mixture of 488 (5.7 g, 15.18 mmol) in EtOH (40 mL) and water (10 mL) was added KOH (1.7 g, 30.36 mmol). The mixture was stirred at 50° C. under Ar for 64 h. After concentration under vacuum, the residue was dissolved with water (80 mL) and extracted with ethyl acetate (50 mL*3). The aqueous phase was acidified with conc. HCl to pH=6 and concentrated in vacuo. The residue was washed with dichloromethane/methanol (20/1, 50 mL*5). The organic phase was concentrated under vacuum to give 489 (5 g, ˜100% yield), which was used in the next step without further purification. MS-ESI (M+H⁺): 322.2.

### Procedure for the Synthesis of 491

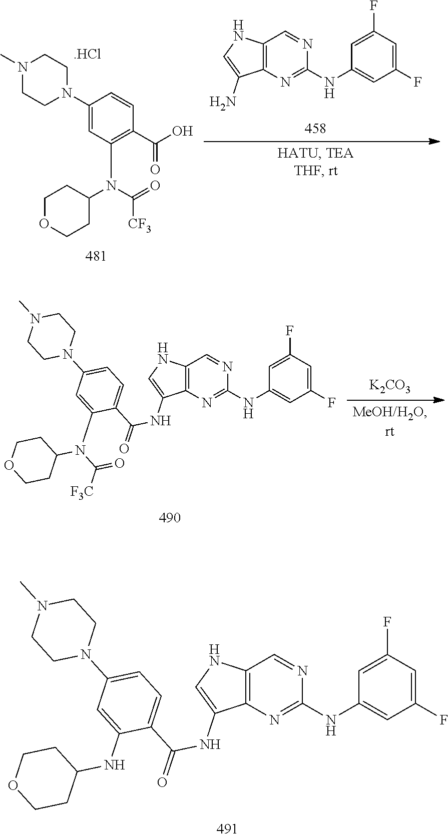

### N-(2-((3,5-difluorophenyl)amino)-5H-pyrrolo[3,2-d]pyrimidin-7-yl)-4-(4-methylpiperazin-1-yl)-2-(2,2,2-trifluoro-N-(tetrahydro-2H-pyran-4-yl)acetamido)benzamide (490)

To the mixture of 458 (60 mg, 0.23 mmol) and 481 (95 mg, 0.23 mmol) in THF (3 mL) were added TEA (0.1 mL, 0.69 mmol) and HATU (96 mg, 0.25 mmol). The mixture was stirred at 25° C. under Ar for 16 h. The mixture was diluted with H₂O (10 mL) and extracted with ethyl acetate (30 mL*3). The organic phase was dried over Na₂SO₄, filtered and concentrated. The residue was purified by Prep-TLC (dichloromethane/methanol=10/1) to give 490 (90 mg, 59.49% yield) as yellow solid. MS-ESI (M+H)⁺: 659.2.

### N-(2-((3,5-difluorophenyl)amino)-5H-pyrrolo[3,2-d]pyrimidin-7-yl)-4-(4-methylpiperazin-1-yl)-2-((tetrahydro-2H-pyran-4-yl)amino)benzamide (491)

To the mixture of 490 (90 mg, 0.14 mmol) in MeOH (2 mL) and H₂O (0.5 mL) was added K₂CO₃ (35 mg, 0.27 mmol). The mixture was stirred at 25° C. under Ar for 16 h. The mixture was concentrated and the residue was purified by Prep-HPLC (MeCN/0.05% NH4OH in water from 0 to 100%) to give 491 (32 mg, 41.62% yield) as white solid. MS-ESI (M+H)⁺: 563.3. ¹H NMR (400 MHz, DMSO-d₆) δ 11.41 (s, 1H), 9.72 (s, 1H), 9.42 (s, 1H), 8.70 (s, 1H), 8.28 (d, J=7.6 Hz, 1H), 7.91 (d, J=2.0 Hz, 1H), 7.76 (d, J=9.0 Hz, 1H), 7.68 (d, J=9.2 Hz, 2H), 6.57 (t, J=9.2 Hz, 1H), 6.23 (d, J=8.8 Hz, 1H), 6.14 (s, 1H), 3.84-3.77 (m, 2H), 3.68-3.62 (m, 1H), 3.49-3.46 (m, 2H), 3.27-3.21 (m, 4H), 2.46-2.36 (m, 4H), 2.22 (s, 3H), 1.93 (d, J=11.8 Hz, 2H), 1.39-1.30 (m, 2H).

### Procedure for the Synthesis of 493

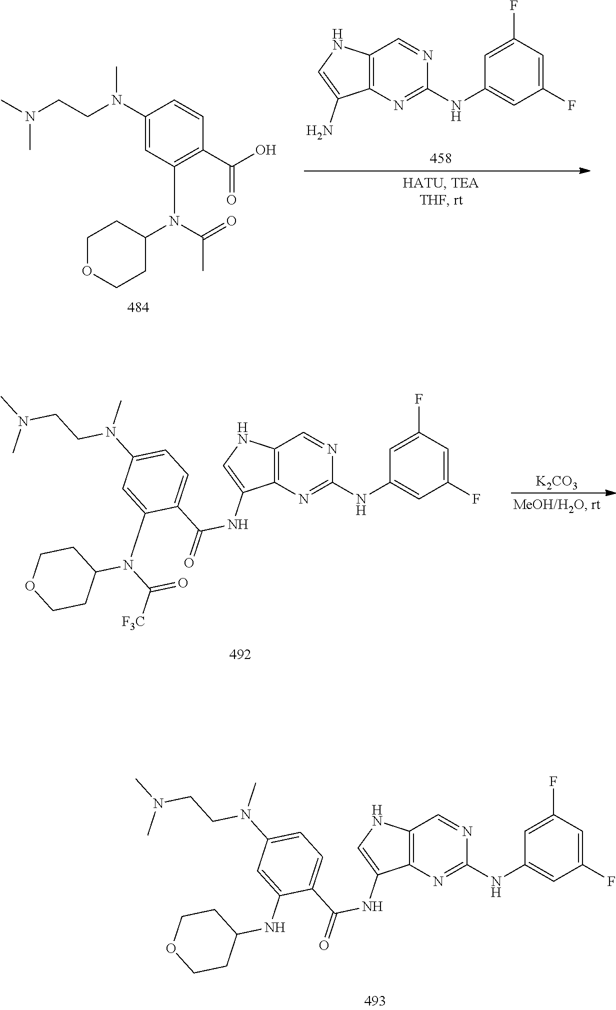

### N-(2-((3,5-difluorophenyl)amino)-5H-pyrrolo[3,2-d]pyrimidin-7-yl)-4-((2-(dimethylamino)ethyl)(methyl)amino)-2-(2,2,2-trifluoro-N-(tetrahydro-2H-pyran-4-yl)acetamido)benzamide (492)

To the mixture of 458 (75 mg, 0.29 mmol) and 484 (132 mg, 0.32 mmol) in THF (4 mL) were added TEA (0.12 mL, 0.86 mmol) and HATU (120 mg, 0.32 mmol). The mixture was stirred at 25° C. under Ar for 16 h. The mixture was quenched with H₂O (10 mL), extracted with ethyl acetate (30 mL*3). The organic phase was dried by Na₂SO₄, filtered and concentrated under reduced pressure. The residue was purified by Prep-TLC (dichloromethane/methanol=10/1) to give 492 (100 mg, 52.7% yield) as white solid. MS-ESI (M+H)⁺: 661.2.

### N-(2-((3,5-difluorophenyl)amino)-5H-pyrrolo[3,2-d]pyrimidin-7-yl)-4-((2-(dimethylamino)ethyl)(methyl)amino)-2-((tetrahydro-2H-pyran-4-yl)amino)benzamide (493)

To a mixture of 492 (100 mg, 0.15 mmol) in MeOH (2 mL) and H₂O (0.5 mL) was added K₂CO₃ (42 mg, 0.30 mmol). The mixture was stirred at 25° C. under Ar for 16 h. The reaction was detected by LCMS. The mixture was concentrated. The residue was purified by Prep-HPLC (NH₃—H₂O) to give 493 (12 mg, 14.04% yield) as white solid. MS-ESI (M+H)⁺: 565.3. ¹H NMR (400 MHz, DMSO-d₆) δ 11.41 (s, 1H), 9.74 (s, 1H), 9.30 (s, 1H), 8.70 (s, 1H), 8.35 (d, J=7.3 Hz, 1H), 7.90 (s, 1H), 7.75-7.68 (m, 3H), 6.58 (t, J=9.0 Hz, 1H), 6.01 (d, J=8.2 Hz, 1H), 5.87 (s, 1H), 3.83 (d, J=11.5 Hz, 2H), 3.66-3.53 (m, 1H), 3.45 (t, J=9.9 Hz, 4H), 2.97 (s, 3H), 2.39 (t, J=7.0 Hz, 2H), 2.20 (s, 6H), 1.96 (d, J=11.6 Hz, 2H), 1.44-1.31 (m, 2H).

### Procedure for the Synthesis of 494

### N-(2-((3,5-difluorophenyl)amino)-5H-pyrrolo[3,2-d]pyrimidin-7-yl)-2-((3-methoxypropyl)(methyl)amino)-4-(4-methylpiperazin-1-yl)benzamide (494)

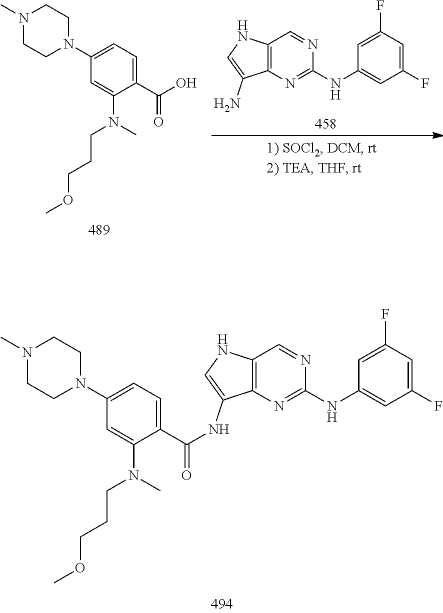 [SMILES: COCCCN(C)c1cc(N2CCN(C)CC2)ccc1C(=O)O ; Nc1c[nH]c2cnc(Nc3cc(F)cc(F)c3)nc12 ; COCCCN(C)c1cc(N2CCN(C)CC2)ccc1C(=O)Nc1c[nH]c2cnc(Nc3cc(F)cc(F)c3)nc12]

To the mixture of 489 (180 mg, 0.56 mmol) in dichloromethane (2 mL) was added SOCl₂ (0.08 mL, 1.12 mmol) under Ar. The mixture was stirred at 25° C. for 3 h. The mixture was concentrated in vacuo. The residue was dissolved with dichloromethane (2 mL). To the mixture were added 458 (146 mg, 0.56 mmol) in THF (2 mL) and TEA (0.12 mL, 0.84 mmol). The mixture was stirred at 25° C. under Ar for 48 h. The reaction was concentrated and the residue was purified by Prep-HPLC (NH₃—H₂O) to give 494 (24.52 mg, 0.043 mmol, 7.75% yield) as brown solid. MS-ESI (M+H⁺): 565.2. ¹H NMR (400 MHz, DMSO-d₆) δ 12.74 (s, 1H), 11.35 (s, 1H), 9.62 (s, 1H), 8.72 (s, 1H), 8.38 (s, 1H), 8.01 (d, J=8.8 Hz, 1H), 7.59 (d, J=8.7 Hz, 2H), 6.96 (s, 1H), 6.87 (d, J=8.6 Hz, 1H), 6.65 (t, J=9.2 Hz, 1H), 3.32-3.28 (m, 4H), 3.16 (t, J=6.1 Hz, 2H), 3.13-3.07 (m, 2H), 2.91 (s, 3H), 2.87 (s, 3H), 2.45 (s, 4H), 2.23 (s, 3H), 1.71-1.58 (m, 2H).

### Procedure for the Synthesis of 497

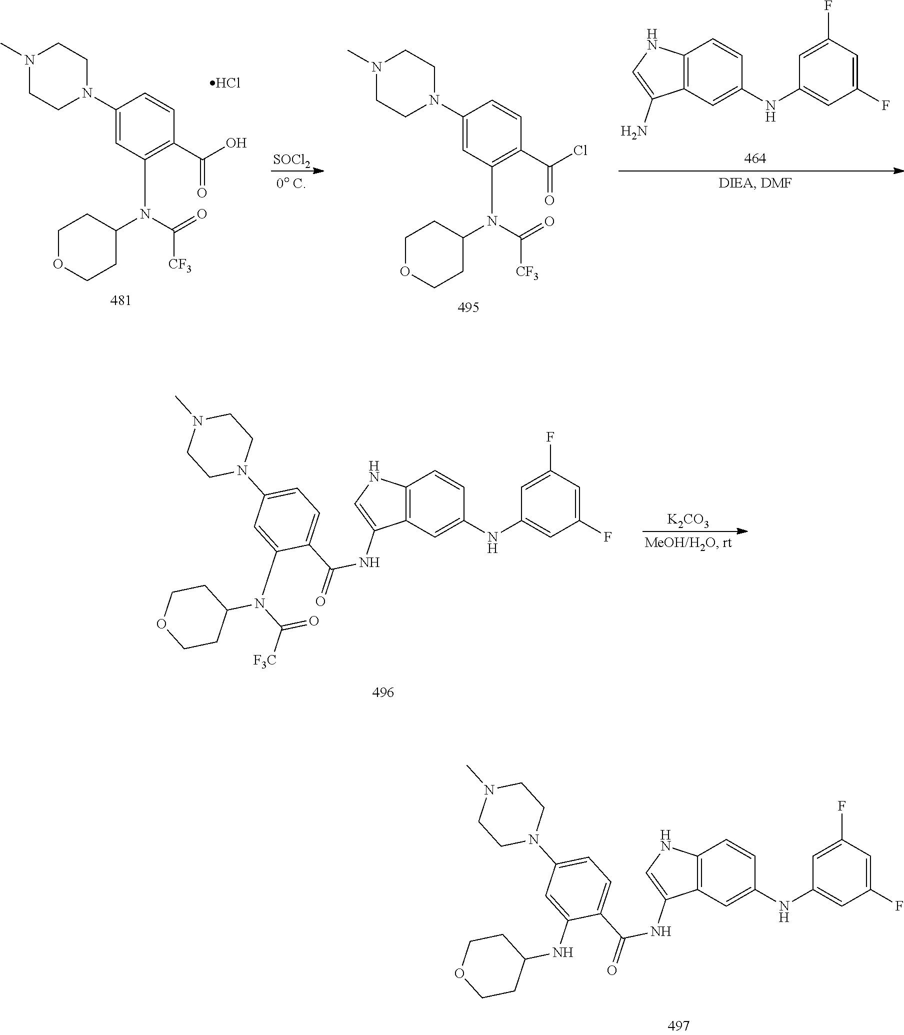

### 4-(4-methylpiperazin-1-yl)-2-(2,2,2-trifluoro-N-(tetrahydro-2H-pyran-4-yl)acetamido)benzoyl chloride (495)

A solution of 481 (180 mg, 0.61 mmol) in SOCl₂ (5 mL) was stirred at 0° C. for 10 min. The mixture was concentrated in vacuo to give crude acid chloride 495, which was dissolved in dichloromethane (3 mL) and used for next step without further purification.

### N-(5-((3,5-difluorophenyl)amino)-1H-indol-3-yl)-4-(4-methylpiperazin-1-yl)-2-(2,2,2-trifluoro-N-(tetrahydro-2H-pyran-4-yl)acetamido)benzamide (496)

To a mixture of 464 (164 mg, 0.57 mmol) in DMF (5 mL) was added 495 (250 mg, 0.57 mmol) and DIEA (0.5 mL). The mixture was stirred at room temperature for 1 h. After diluting with (200 mL), the organic phase was washed with NaCl (150 mL), dried over Na₂SO₄. After concentration, the crude title product 496 (200 mg) was obtained, which was used to the next batch directly.

### N-(5-((3,5-difluorophenyl)amino)-1H-indol-3-yl)-4-(4-methylpiperazin-1-yl)-2-((tetrahydro-2H-pyran-4-yl)amino)benzamide (497)

A mixture of 496 (crude 200 mg, 0.3 mmol) and K₂CO₃ (12 mg, 0.9 mmol) in MeOH (5 mL) was stirred at room temperature for 2 h. After filtration, the filtrate was purified by pre-HPLC (NH₄OH condition) to give the title product 497 as a white solid (50.2 mg, 15% yield for two steps) m/z (MS-ESI) (M+H)⁺: 561.2. ¹H NMR (400 MHz, DMSO-d₆) δ 10.85 (s, 1H), 9.62 (s, 1H), 8.35 (s, 1H), 8.10 (d, J=6.9 Hz, 1H), 7.67-7.64 (m, 3H), 7.34 (d, J=8.4 Hz, 1H), 6.91 (d, J=8.1 Hz, 1H), 6.44 (d, J=8.9 Hz, 2H), 6.33 (s, 1H), 6.23 (d, J=8.0 Hz, 1H), 6.14 (s, 1H), 3.83 (d, J=10.5 Hz, 2H), 3.64-3.58 (m, 2H), 3.48-3.44 (m, 3H), 3.23 (s, 3H), 2.43 (s, 3H), 2.22 (s, 3H), 1.96-1.93 (m, 2H), 1.47-1.21 (m, 2H).

### Procedure for the Synthesis of 500

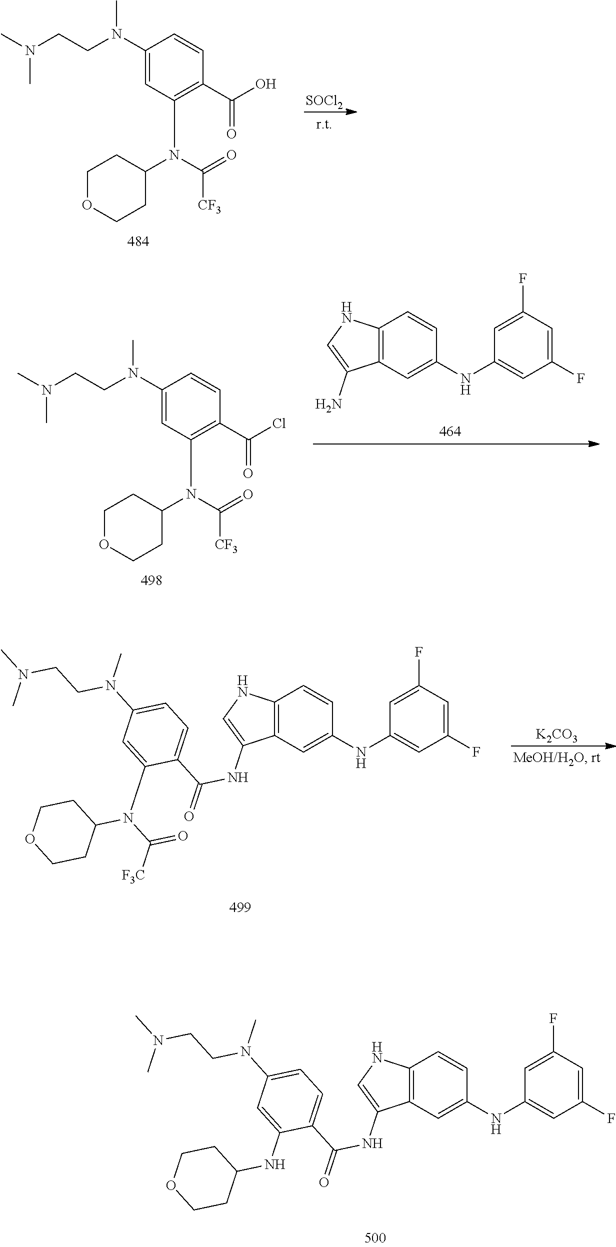

### 4-((2-(dimethylamino)ethyl)(methyl)amino)-2-(2,2,2-trifluoro-N-(tetrahydro-2H-pyran-4-yl)acetamido)benzoyl chloride (498)

A solution of 484 (350 mg, 0.66 mmol) in SOCl₂ (5 mL) was stirred at room temperature for 2 h. After concentration under vacuum, the residue 498 (300 mg) was used to next batch directly.

### N-(5-((3,5-difluorophenyl)amino)-1H-indol-3-yl)-4-((2-(dimethylamino)ethyl)(methyl)amino)-2-(2,2,2-trifluoro-N-(tetrahydro-2H-pyran-4-yl)acetamido)benzamide (499)

Synthesis of 499 was similar to that of 496. After concentration, crude title product 499 was used to next step without further purification.

### N-(5-((3,5-difluorophenyl)amino)-1H-indol-3-yl)-4-((2-(dimethylamino)ethyl)(methyl)amino)-2-((tetrahydro-2H-pyran-4-yl)amino)benzamide (500)

To a solution of 499 (crude, 250 mg, 0.38 mmol) in MeOH/H₂O (5 mL/1 mL) was added K₂CO₃ (80 mg, 0.57 mmol). The mixture was stirred at room temperature for 2 h. The solid was filtered off and the filtrate was purified by pre-HPLC to give product 500 (30.06 mg). m/z. MS-ESI (M+H)⁺: 562.3. ¹H NMR (400 MHz, DMSO-d₆) δ 10.83 (s, 1H), 9.50 (s, 1H), 8.34 (s, 1H), 8.17 (d, J=7.4 Hz, 1H), 7.65-7.56 (m, 3H), 7.34 (d, J=8.5 Hz, 1H), 6.90 (dd, J=8.5, 1.7 Hz, 1H), 6.43 (d, J=8.7 Hz, 2H), 6.32 (t, J=9.3 Hz, 1H), 5.99 (d, J=8.9 Hz, 1H), 5.86 (s, 1H), 3.88-3.80 (m, 3H), 3.56 (s, 3H), 2.95 (br s, 4H), 2.40-2.34 (m, 2H), 2.18 (s, 6H), 1.97-1.94 (m, 2H), 1.41-1.33 (m, 2H).

### Procedure for the Synthesis of 501

### N-(5-((3,5-difluorophenyl)amino)-1H-indol-3-yl)-2-((3-methoxypropyl)(methyl)amino)-4-(4-methylpiperazin-1-yl)benzamide (501)

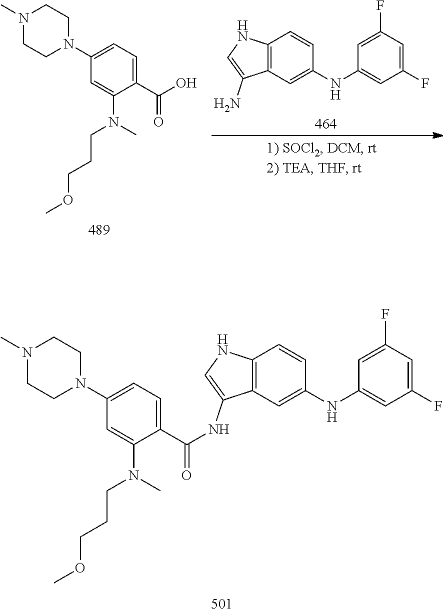 [SMILES: COCCCN(C)c1cc(N2CCN(C)CC2)ccc1C(=O)O ; Nc1c[nH]c2ccc(Nc3cc(F)cc(F)c3)cc12 ; COCCCN(C)c1cc(N2CCN(C)CC2)ccc1C(=O)Nc1c[nH]c2ccc(Nc3cc(F)cc(F)c3)cc12]

To the mixture of 489 (200 mg, 0.62 mmol) in dichloromethane (10 mL) was added SOCl₂ (0.09 mL, 1.24 mmol) under Ar. The mixture was stirred at 25° C. for 3 h. The mixture was concentrated in vacuo. The residue was dissolved with dichloromethane (6 mL). To the mixture were added 464 (161 mg, 0.62 mmol) in THF (2 mL) and TEA (0.13 mL, 0.93 mmol). The mixture was stirred at 25° C. under Ar for 16 h. After concentration, the residue was purified by Prep-HPLC (NH₃—H₂O) to give 501 (21.78 mg, 0.038 mmol, 6.22% yield) as brown solid. MS-ESI (M+H⁺): 563.3. ¹H NMR (400 MHz, CDCl₃+CD3OD) δ 8.15 (d, J=8.8 Hz, 1H), 7.89 (s, 1H), 7.32 (dd, J=7.1, 5.3 Hz, 2H), 6.97 (dd, J=8.6, 2.0 Hz, 1H), 6.81 (d, J=8.2 Hz, 2H), 6.43 (dd, J=10.0, 2.1 Hz, 2H), 6.18-6.09 (m, 1H), 3.66-3.47 (m, 4H), 3.25 (t, J=5.9 Hz, 2H), 3.21-3.02 (m, 9H), 2.79 (s, 3H), 2.73 (s, 3H), 1.83-1.64 (m, 2H).

### Procedure for the Synthesis of 503

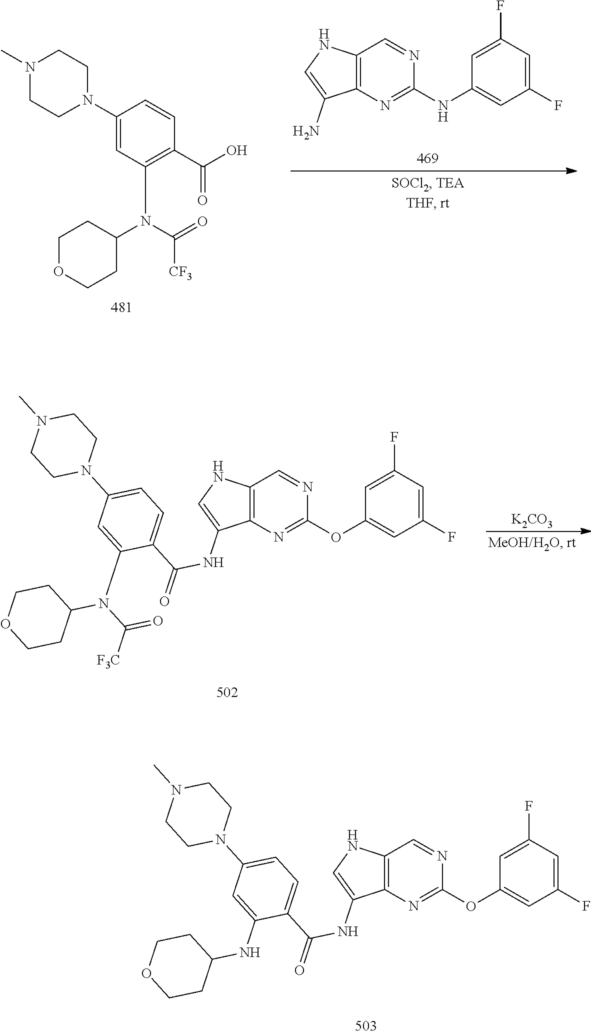

### N-(2-(3,5-difluorophenoxy)-5H-pyrrolo[3,2-d]pyrimidin-7-yl)-4-(4-methylpiperazin-1-yl)-2-(2,2,2-trifluoro-N-(tetrahydro-2H-pyran-4-yl)acetamido)benzamide (502)

To a mixture of 481 (98 mg, 0.23 mmol) in THF (1 mL) was added SOCl₂ (56 mg, 0.47 mmol) under Ar. The mixture was stirred at 25° C. under Ar for 2 h. To the mixture was added 469 (82 mg, 0.16 mmol) and TEA (63 mg, 0.63 mmol) under Ar. The mixture was stirred at 25° C. under Ar for 40 h. The reaction was quenched with H₂O (0.5 mL) and concentrated. The residue was purified by prep-TLC (dichloromethane/methanol=10/1) to give 502 (40 mg, 38.78% yield) as yellow solid. MS-ESI (M+H)⁺: 660.2.

### N-(2-(3,5-difluorophenoxy)-5H-pyrrolo[3,2-d]pyrimidin-7-yl)-4-(4-methylpiperazin-1-yl)-2-((tetrahydro-2H-pyran-4-yl)amino)benzamide (503)

To the mixture of 502 (40 mg, 0.061 mmol) in MeOH (1 mL) and H₂O (0.5 mL) was added K₂CO₃ (17 mg, 0.121 mmol). The mixture was stirred at 25° C. under Ar for 16 h. The mixture was concentrated. The residue was purified by Prep-HPLC (FA) and basified to give 503 (11.26 mg, 32.95% yield) as white solid. MS-ESI (M+H)⁺: 564.2. ¹H NMR (400 MHz, DMSO-d₆) δ 9.48 (s, 1H), 8.71 (s, 1H), 8.22 (s, 1H), 8.12 (s, 1H), 7.73 (d, J=8.0 Hz, 1H), 7.01 (s, 1H), 6.92 (d, J=6.7 Hz, 2H), 6.22 (d, J=7.1 Hz, 1H), 6.13 (s, 1H), 3.81 (br, 2H), 3.62 (br, 2H), 3.23-3.20 (m, 3H), 3.17 (br, 2H), 2.43 (br, 4H), 2.22 (s, 3H), 1.93 (d, J=10.8 Hz, 2H), 1.33-1.31 (m, 2H).

### Procedure for the Synthesis of 505

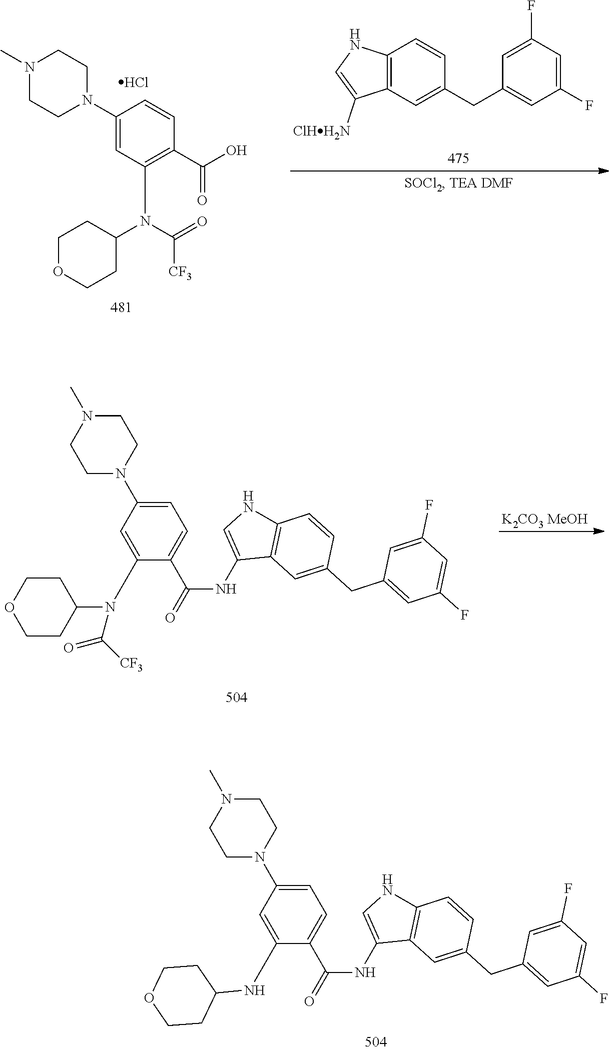

### N-(5-(3,5-difluorobenzyl)-1H-indol-3-yl)-4-(4-methylpiperazin-1-yl)-2-(2,2,2-trifluoro-N-(tetrahydro-2H-pyran-4-yl)acetamido)benzamide (504)

A solution of 481 (180 mg, 0.61 mmol) in SOCl₂ (5 mL) was stirred at 0° C. for 10 min. The mixture was concentrated in vacuo to give crude acid chloride, which was dissolved in dichloromethane (3 mL). Then a solution of 475 (270 mg, 0.65 mmol) and TEA (1 mL) in DMF (5 mL) was added in. After 1 h, the reaction mixture was diluted with 30 mL of water, extracted with ethyl acetate (30 mL×2). The combined organic layers were concentrated to give the title product 504 as a black solid (200 mg yield 52.4%), which was used in next step directly. MS-ESI (M+H)⁺:656.2.

### N-(5-(3,5-difluorobenzyl)-1H-indol-3-yl)-4-(4-methylpiperazin-1-yl)-2-((tetrahydro-2H-pyran-4-yl)amino)benzamide (505)

A mixture of 504 (crude 200 mg, 0.31 mmol) and K₂CO₃ (75 mg, 0.62 mmol) in MeOH (5 mL) was stirred at room temperature for 1 h. The reaction mixture was concentrated. The residue was treated with water/EA (100 mL/200 mL). The organic phase was concentrated to give a residue which was purified by pre-HPLC (MeCN/0.05% formic acid in water from 0 to 100%)) to give the title product 505 as a white solid (22.4 mg, 12.9%). MS-ESI (M+H)⁺: 560.2. ¹H NMR (400 MHz, CDCl₃) δ 8.07 (s, 1H), 7.93 (d, J=6.4 Hz, 1H), 7.67 (d, J=11.6 Hz, 2H), 7.43 (d, J=8.6 Hz, 1H), 7.28-7.20 (m, 2H), 6.94 (d, J=8.0 Hz, 1H), 6.64 (d, J=6.2 Hz, 2H), 6.55 (d, J=8.7 Hz, 1H), 6.17 (d, J=8.1 Hz, 1H), 6.06 (s, 1H), 4.06-3.81 (m, 4H), 3.50-3.48 (m, 3H), 3.27 (br s, 4H), 2.56 (br s, 4H), 2.33 (s, 3H), 1.97-1.95 (m, 2H), 1.57-1.54 (m, 2H).

### Procedure for the Synthesis of 507

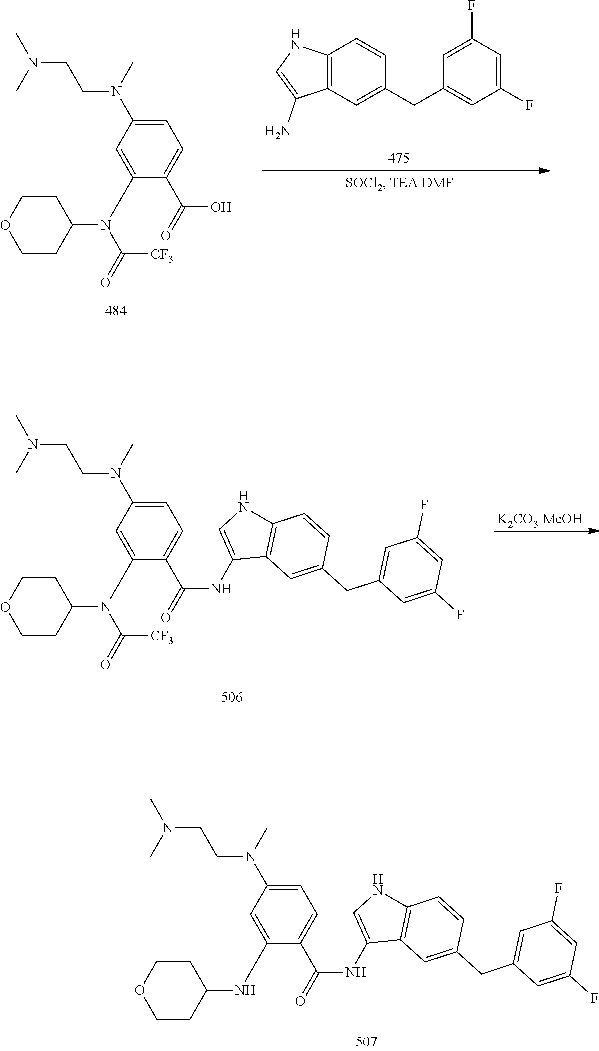 [SMILES: CN(C)CCN(C)c1ccc(C(=O)O)c(N(C(=O)C(F)(F)F)C2CCOCC2)c1 ; Nc1c[nH]c2ccc(Cc3cc(F)cc(F)c3)cc12 ; CN(C)CCN(C)c1ccc(C(=O)Nc2c[nH]c3ccc(Cc4cc(F)cc(F)c4)cc23)c(N(C(=O)C(F)(F)F)C2CCOCC2)c1 ; "K2CO3 MeOH" ; CN(C)CCN(C)c1ccc(C(=O)Nc2c[nH]c3ccc(Cc4cc(F)cc(F)c4)cc23)c(NC2CCOCC2)c1]

### N-(5-(3,5-difluorobenzyl)-1H-indol-3-yl)-4-((2-(dimethylamino)ethyl)(methyl)amino)-2-(2,2,2-trifluoro-N-(tetrahydro-2H-pyran-4-yl)acetamido)benzamide (506)

Synthesis of 506 was similar to that of 504. Crude title product 506 as a black solid (180.0 mg, 57.4%) was obtained, which was used in next step without further purification. MS-ESI (M+H)⁺: 658.2.

### N-(5-(3,5-difluorobenzyl)-1H-indol-3-yl)-4-(2-(dimethylamino)ethyl)(methyl amino)-2-((tetrahydro-2H-pyran-4-yl)amino) benzamide (507)

A mixture of 506 (crude 180.0 mg, 0.27 mmol) and K₂CO₃ (69.2 mg, 0.54 mmol) in MeOH (5 mL) was stirred at room temperature for 1 h. The reaction mixture was filtered and the filtrate was purified by pre-HPLC (MeCN/0.05% formic acid in water from 0 to 100%)) to give the title product 507 (54.99 mg, yield 36.3%) as a white solid ¹H NMR (400 MHz, CD₃OD-d₄) δ 7.67 (d, J=8.9 Hz, 1H), 7.49 (d, J=5.0 Hz, 2H), 7.30 (d, J=8.3 Hz, 1H), 6.98 (dd, J=8.4, 1.5 Hz, 1H), 6.80 (d, J=6.6 Hz, 2H), 6.71-6.66 (m, 1H), 6.14 (dd, J=8.9, 2.4 Hz, 1H), 5.99 (d, J=2.2 Hz, 1H), 4.05 (s, 2H), 3.96-3.03 (m, 2H), 3.68-3.62 (m, 1H), 3.61-3.51 (m, 4H), 3.02 (s, 3H), 2.57-2.51 (m, 2H), 2.33 (s, 6H), 2.06-2.03 (m, 2H), 1.57-1.52 (m, 2H). MS-ESI (M+H)⁺: 562.5.

### Procedure for the Synthesis of 508

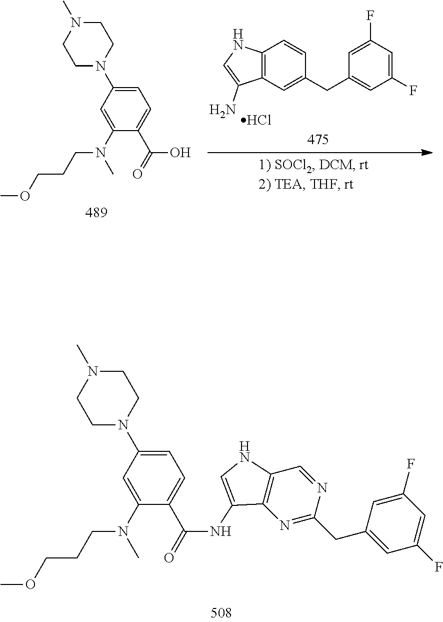 [SMILES: COCCCN(C)c1cc(N2CCN(C)CC2)ccc1C(=O)O ; Nc1c[nH]c2ccc(Cc3cc(F)cc(F)c3)cc12 ; Cl ; COCCCN(C)c1cc(N2CCN(C)CC2)ccc1C(=O)Nc1c[nH]c2cnc(Cc3cc(F)cc(F)c3)nc12]

### N-(5-(3,5-difluorobenzyl)-1H-indol-3-yl)-2-((3-methoxypropyl)(methyl)amino)-4-(4-methylpiperazin-1-yl)benzamide (508)

To the mixture of 489 (150 mg, 0.47 mmol) in dichloromethane (2 mL) was added SOCl₂ (0.07 mL, 0.93 mmol) at 25° C. under Ar. The mixture was stirred at 25° C. under Ar for 2 h. The mixture was concentrated in vacuo. The residue was dissolved with dichloromethane (2 mL). To the mixture was added 475 (120 mg, 0.47 mmol) in THF (2 mL) and TEA (0.07 mL, 0.47 mmol). The mixture was stirred at 25° C. under Ar for 16 h. The mixture was concentrated in vacuo and purified by Prep-HPLC (FA) and Prep-HPLC (NH₃H₂O) to give 508 (7.89 mg, 0.014 mmol, 3.01% yield) as white solid. MS-ESI (M+H)⁺: 562.4; ¹H NMR (400 MHz, DMSO-d₆) δ 12.93 (s, 1H), 10.82 (s, 1H), 8.14-7.92 (m, 1H), 7.88 (s, 1H), 7.45-7.29 (m, 1H), 7.22 (s, 1H), 7.15-6.95 (m, 5H), 6.95-6.80 (m, 1H), 4.15 (s, 2H), 3.25-3.19 (m, 2H), 3.15-3.08 (m, 2H), 3.07-2.94 (m, 4H), 2.76 (s, 3H), 2.59-2.52 (m, 7H), 2.27 (s, 3H), 1.72-1.47 (m, 2H).

### Synthesis of Compounds of Formula II

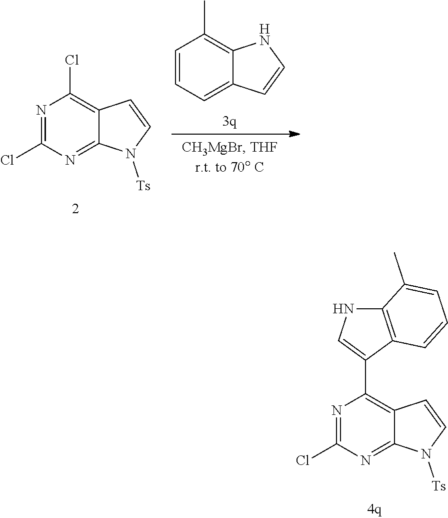

### 2-chloro-4-(1,7-dimethyl-1H-indol-3-yl)-7-tosyl-7H-pyrrolo[2,3-d]pyrimidine (4q)

CH₃MgBr (3.0M in 2-methyltetrehydrofuran, 2.7 ml, 8.1 mmol) was added dropwise over 10 minutes to a solution of 7-methyl-1H-indole 3q (1.06 g, 8.01 mmol) in tetrahydrofuran (20 ml) at 0° C. The solution was then stirred at 0° C. for 0.5 h. Intermediate 2 (1.72 g, 5.08 mmol) was added. The ice bath was removed, then the solution was stirred at room temperature for 1 h. The mixture was heated 70° C. overnight. The mixture was then cooled to room temperature and saturated NH₄Cl was added (50 ml). The mixture was extracted with ethyl acetate (50 ml). The organic layer was washed with water (30 ml*3) and concentrated under reduced pressure to give a residue. The residue was purified by column (petroleum ether/ethyl acetate=10/1) to give the compound 4q (0.47 g, yield 21.1%) as a solid.

### Intermediate 4s

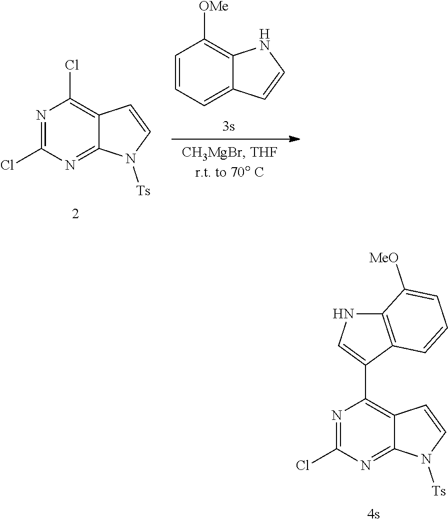 [SMILES: Cc1ccc(S(=O)(=O)n2ccc3c(Cl)nc(Cl)nc32)cc1 ; COc1cccc2cc[nH]c12 ; COc1cccc2c(-c3nc(Cl)nc4c3ccn4S(=O)(=O)c3ccc(C)cc3)c[nH]c12]

### 2-chloro-4-(7-methoxy-1H-indol-3-yl)-7-tosyl-7H-pyrrolo[2,3-d]pyrimidine (4s)

CH₃MgBr (3.0M in 2-methyltetrehydrofuran, 2.7 ml, 8.1 mmol) was added dropwise over 10 minutes to a solution of 7-methoxy-1H-indole 3s (1.1 g, 7.47 mmol) in tetrahydrofuran (20 ml) at 0° C. The solution was then stirred at 0° C. for 0.5 h. Intermediate 2 (1.74 g, 5.08 mmol) was added. The ice bath was removed, then the solution was stirred at room temperature for 1 h. The mixture was heated 70° C. overnight. The mixture was then cooled to room temperature and saturated NH₄Cl was added (50 ml). The mixture was extracted with ethyl acetate (50 ml). The organic layer was washed with water (30 ml*3) and concentrated under reduced pressure to give a residue. The residue was purified by column (petroleum ether/ethyl acetate=10/1) to give the compound 4s (0.20 g, yield 8.6%) as a solid.

### Intermediate 17i

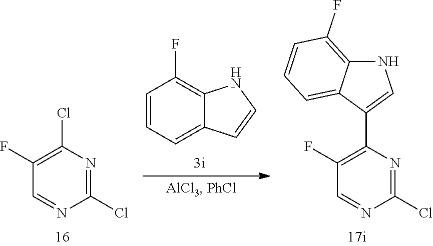

### 3-(2-chloro-5-fluoropyrimidin-4-yl)-7-fluoro-1H-indole (17i)

AlCl₃ (4.79 g, 35.93 mmol) was added into a solution of 16 (2.00 g, 11.98 mmol) and 7-fluoro-Indole 3i (1.78 g, 13.18 mmol) in PhCl (50 mL) at 20° C. Then adjusted the reaction to 80° C. and stirred overnight. The reaction was then cooled to room temperature and filtered through silica gel. The filtrate was concentrated and the residual was slurried with ethyl acetate to afford 17i (1.75 g, yield: 55.0%) as a gray solid. LC-MS (ESI): m/z 266.4 [M+H]⁺.

### Intermediate 17q

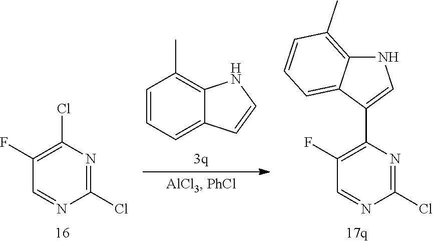

### 3-(2-chloro-5-fluoropyrimidin-4-yl)-7-methyl-1H-indole (17q)

AlCl₃ (4.79 g, 35.93 mmol) was added into a solution of 16 (2.00 g, 11.98 mmol) and 7-methyl-Indole (1.73 g, 13.18 mmol) in PhCl (50 mL) at 20° C. Then adjusted the reaction to 80° C. and stirred overnight. The reaction was then cooled to room temperature and filtered through silica gel. The filtrate was concentrated and the residual was slurried with (petroleum ether/ethyl acetate=3/1) to afford 17q (1.25 g, yield: 39.9%) as a gray solid. LC-MS (ESI): m/z 262.2 [M+H]⁺.

### Intermediate 17s

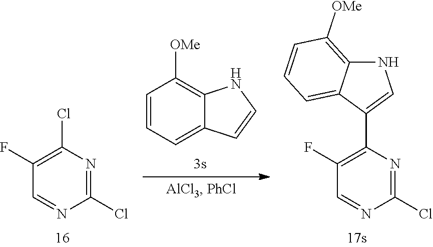

### 3-(2-chloropyrimidin-4-yl)-7-fluoro-1H-indole (17s)

To a solution of 3s (2.00 g, 13.59 mmol) in PhCl (20 mL), AlCl₃ (2.00 g, 14.95 mmol) and 2,4-dichloro-5-fluoropyrimidine (2.29 g, 13.72 mmol) were added. The mixture was stirred at 50° C. for 3.5 h. The mixture was quenched by H₂O (40 mL) and was filtered off. The filter cake was washed with ethyl acetate (15 mL×2), and dried in an air-circulating oven at 40° C. overnight to give the product 17s (2.41 g, yield: 63.9%) as a brownish red solid.

### Intermediate 510i

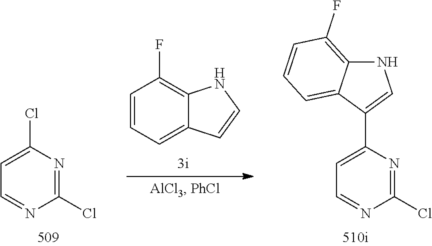 [SMILES: Clc1ccnc(Cl)n1 ; Fc1cccc2cc[nH]c12 ; Fc1cccc2c(-c3ccnc(Cl)n3)c[nH]c12]

### 3-(2-chloropyrimidin-4-yl)-7-fluoro-1H-indole (510i)

To a solution of 3i (1.00 g, 7.40 mmol) in PhCl (20 mL), AlCl₃ (1.09 g, 8.14 mmol) and 2,4-dichloropyrimidine 509 (1.10 g, 7.40 mmol) were added. The mixture was stirred at 50° C. for 4 h. The mixture was quenched by H₂O (30 mL) and was filtered off. The filter cake was washed with ethyl acetate (10 mL×2), and dried in an air-circulating oven at 40° C. overnight to give the product 510i (0.70 g, yield: 38.2%) as a yellow solid.

### Intermediate 510q

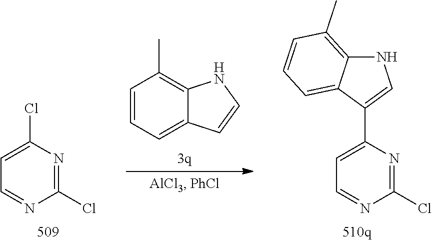

### 3-(2-chloropyrimidin-4-yl)-7-methyl-1H-indole (510g)

To a solution of 3q (1.01 g, 7.70 mmol) in PhCl (15 mL), AlCl₃ (1.02 g, 7.70 mmol) and 2,4-dichloropyrimidine 509 (1.14 g, 7.70 mmol) were added. The mixture was stirred at 60° C. for 2.5 h. The mixture was quenched by H₂O (30 mL) and was filtered off. The filter cake was washed with ethyl acetate (10 mL×2), and dried in an air-circulating oven at 40° C. overnight to give the product 510q (1.40 g, yield: 74.6%) as a red solid.

### Intermediate 510s

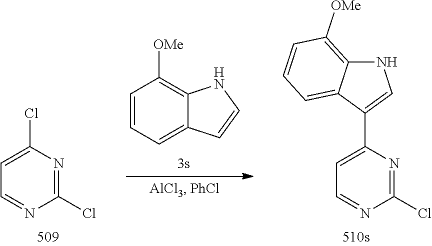 [SMILES: Clc1ccnc(Cl)n1 ; COc1cccc2cc[nH]c12 ; COc1cccc2c(-c3ccnc(Cl)n3)c[nH]c12]

### 3-(2-chloropyrimidin-4-yl)-7-methoxy-1H-indole (510s)

To the mixture of 2,4-dichloropyrimidine 509 (1.8 g, 12.1 mmol) in PhCl (15 mL) were added AlCl₃ (1.8 g, 13.5 mmol). The mixture was stirred at room temperature for 0.5 h, then 7-methoxy-1H-indole 3s (2.5 g, 17 mmol) was added. The mixture was stirred at 85° C. for 3-4 h. The mixture was quenched with water, extracted with dichloromethane. The combined organic phase was concentrated, purified by flash-chromatography (petroleum ether/ethyl acetate=10/1˜3/1) to give the 510s (153 mg, 0.59 mmol, 4.9% yield) as a brown solid.

### Procedure for the Synthesis of 513, 514

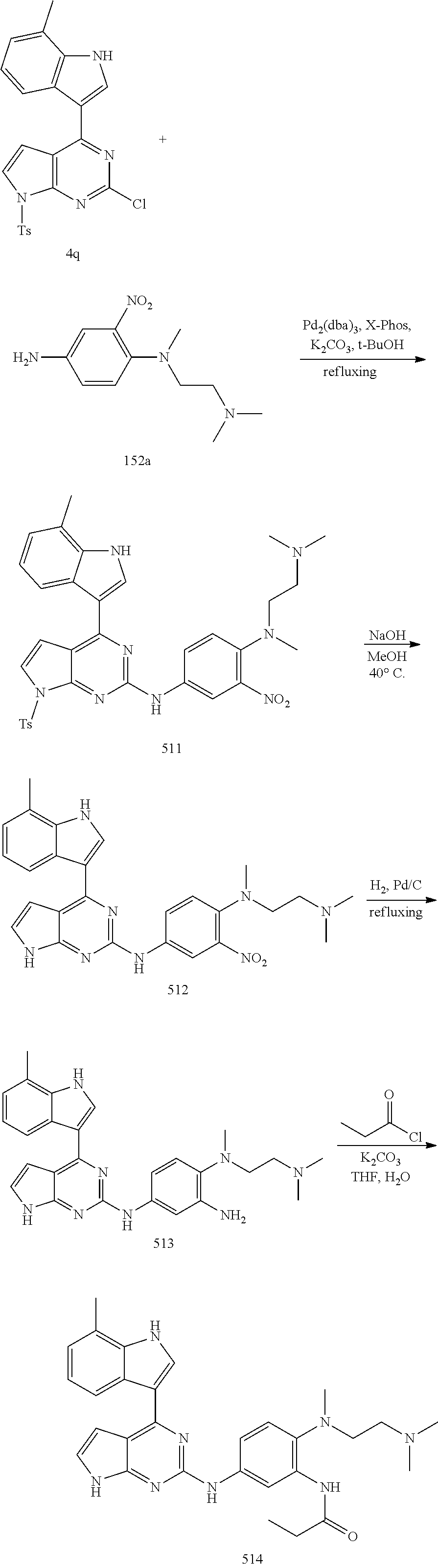

### N¹-(2-(dimethylamino)ethyl)-N¹-methyl-N⁴-(4-(7-methyl-1H-indol-3-yl)-7-tosyl-7H-pyrrolo[2,3-d]pyrimidin-2-yl)-2-nitrobenzene-1,4-diamine (511)

X-Phos (0.02 g), Pd₂(dba)₃ (0.02 g), K₂CO₃ (0.31 g, 2.24 mmol) and 4q (0.47 g, 1.07 mmol) were added to a stirred solution of 152a (0.26 g, 1.09 mmol) in tert-butanol (10 ml). The resulting mixture was heated at refluxing for 7 h. Then the reaction mixture was allowed to cool down. The reaction mixture was filtered and concentrated. The residue was purified by column (methanol/dichloromethane=1/20) to give the compound 511 (0.41 g, yield 60.0%). MS-ESI (M+H)⁺: 638.9.

### N¹-(2-(dimethylamino)ethyl)-N¹-methyl-N⁴-(4-(7-methyl-1H-indol-3-yl)-7H-pyrrolo[2,3-d]pyrimidin-2-yl)-2-nitrobenzene-1,4-diamine (512)

The compound 511 (0.41 g, 0.64 mmol) was mixed and dispersed in methanol (10 ml) at 40° C. NaOH (0.83 g, 20 mmol) was added separately to the reaction mixture. And then the mixture was stirred for a further 0.5 h at 40° C. Then the reaction mixture was allowed to cool down and quench with water (10 ml). The reaction mixture was filtered. The filter cake was washed with water (30*3) and dried under reduce pressure at 40° C. The crude product 512 (0.30 g, yield 96.7%) was directly used for the next reaction without further purification. MS-ESI (M+H)⁺: 485.5.

### N¹-(2-(dimethylamino)ethyl)-N¹-methyl-N⁴-(4-(7-methyl-1H-indol-3-yl)-7H-pyrrolo[2,3-d]pyrimidin-2-yl)benzene-1,2,4-triamine (513)

A mixture of compound 512 (0.11 g, 0.22 mmol) and Pd/C (0.03 g) in methanol (5 ml) was stirred under H₂ atmosphere at room temperature overnight. The reaction mixture was filtered and concentrated under reduce pressure. The residue was purified by column (methanol/dichloromethane=1/20) to give the compound 513 (0.08 g, yield 79.8%). MS-ESI (M+H)⁺:455.5. ¹H NMR (500 MHz, DMSO-d₆) δ 11.78 (s, 1H), 11.36 (s, 1H), 8.72 (s, 1H), 8.70-8.63 (m, 1H), 8.25 (s, 1H), 7.17-7.11 (m, 2H), 7.08 (d, J=2.4 Hz, 1H), 7.08-7.02 (m, 1H), 6.99 (d, J=7.0 Hz, 1H), 6.90 (d, J=8.5 Hz, 1H), 6.79 (d, J=1.8 Hz, 1H), 5.77 (s, 1H), 2.59 (s, 3H), 2.55 (s, 3H), 2.54 (s, 6H), 2.51 (p, J=1.7 Hz, 4H).

### N-(2-((2-(dimethylamino)ethyl)(methyl)amino)-5-((4-(7-methyl-1H-indol-3-yl)-7H-pyrrolo[2,3-d]pyrimidin-2-yl)amino)phenyl)propionamide (514)

The compound 513 (0.06 g, 0.13 mmol), K₂CO₃ (0.04 g, 0.28 mmol), tetrahydrofuran (2 ml) and water (2 ml) were added to the reaction bottle (50 ml). The resulting mixture was stirred at 0° C. Propionyl chloride (0.03 g, 0.32 mmol) was dropped into the reaction mixture. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by preparative thin layer chromatography (dichloromethane/methanol=20/1) to give the compound 514 (32.9 mg, yield 49.6%) MS-ESI (M+H)⁺:511.8. ¹H NMR (500 MHz, DMSO-d₆) δ 11.77 (s, 1H), 11.41 (s, 1H), 9.73 (s, 1H), 9.05 (s, 1H), 8.74-8.61 (m, 1H), 8.34 (s, 1H), 8.30-8.26 (m, 1H), 7.80 (dd, J=8.7, 2.5 Hz, 1H), 7.18 (d, J=8.7 Hz, 1H), 7.14 (dd, J=3.4, 2.4 Hz, 1H), 7.05 (t, J=7.5 Hz, 1H), 6.99 (d, J=7.0 Hz, 1H), 6.84-6.79 (m, 1H), 5.77 (s, 1H), 2.61 (s, 3H), 2.54 (s, 3H), 2.52-2.49 (m, 6H), 2.46 (s, 6H), 1.11 (t, J=7.5 Hz, 3H).

### Procedure for the Synthesis of 518

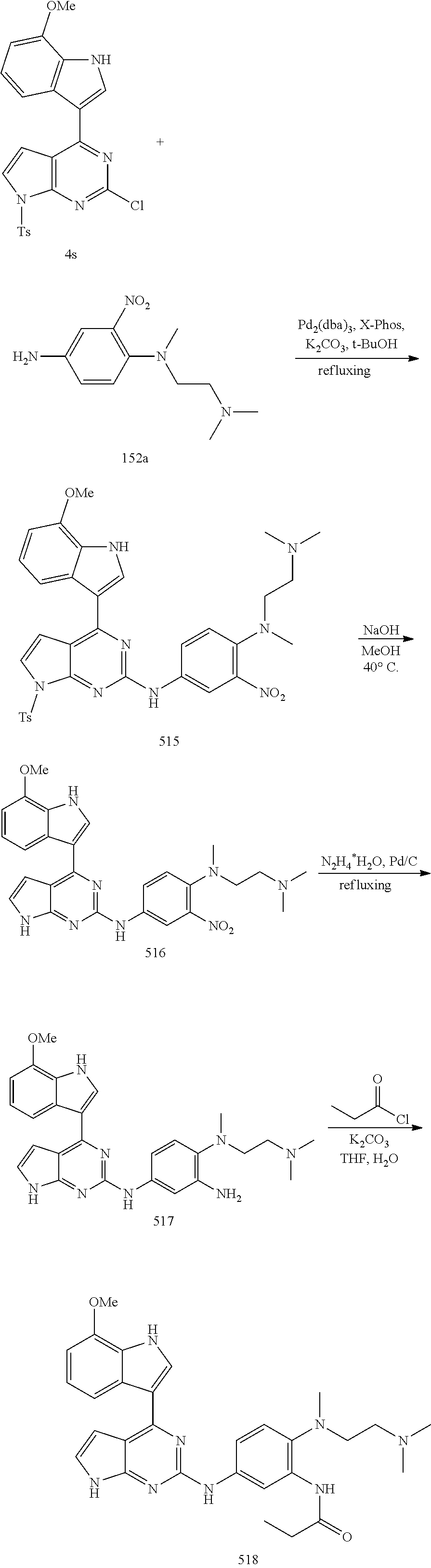

### N¹-(2-(dimethylamino)ethyl)-N⁴-(4-(7-methoxy-1H-indol-3-yl)-7-tosyl-7H-pyrrolo[2,3-d]pyrimidin-2-yl)-N¹-methyl-2-nitrobenzene-1,4-diamine (515)

X-Phos (0.02 g), Pd₂(dba)₃ (0.02 g), K₂CO₃ (0.13 g, 2.17 mmol) and the compound 4s (0.20 g, 0.44 mmol) was added to a stirred solution of the compound 152a (0.11 g, 0.46 mmol) in tert-butanol (10 ml). The resulting mixture was heated at refluxing for 7 h. Then the reaction mixture was allowed to cool down. The reaction mixture was filtered and concentrated. The residue was purified by column (methanol/dichloromethane=1/20) to give the compound 515 (0.18 g, yield 62.5%). MS-ESI (M+H)⁺: 654.2.

### N¹-(2-(dimethylamino)ethyl)-N⁴-(4-(7-methoxy-1H-indol-3-yl)-7H-pyrrolo[2,3-d]pyrimidin-2-yl)-N¹-methyl-2-nitrobenzene-1,4-diamine (516)

The compound 515 (0.18 g, 0.27 mmol) was mixed and dispersed in methanol (10 ml) at 40° C. NaOH (0.4 g, 10 mmol) was added separately to the reaction mixture. And then the mixture was stirred for a further 0.5 h at 40° C. Then the reaction mixture was allowed to cool down and quench with water (10 ml). The reaction mixture was filtered. The filter cake was washed with water (30 mL*3) and dried under reduce pressure at 40° C. The crude product 516 (0.10 g, yield 73.8%) was directly used for the next reaction without further purification. MS-ESI (M+H)⁺: 501.5.

### N¹-(2-(dimethylamino)ethyl)-N⁴-(4-(7-methoxy-1H-indol-3-yl)-7H-pyrrolo[2,3-d]pyrimidin-2-yl)-N¹-methylbenzene-1,2,4-triamine (517)

A mixture of the compound 516 (0.10 g, 0.2 mmol), Pd/C (10%, 0.01 g) and hydrazine hydrate (80%, 2 ml) in tetrahydrofuran (4 ml) was stirred at refluxing for 8 h. Then the reaction mixture was cool down and filtered. The filtrate was concentrated and purified by column (methanol/dichloromethane=1/10) to give the compound 517 (0.08 g, yield 85.0%). MES-ESI (M+H)⁺:471.5.

### N-(2-((2-(dimethylamino)ethyl)(methyl)amino)-5-((4-(7-methoxy-1H-indol-3-yl)-7H-pyrrolo[2,3-d]pyrimidin-2-yl)amino)phenyl)propionamide (518)

The compound 517 (0.08 g, 0.17 mmol), K₂CO₃ (0.05 g, 0.36 mmol), tetrahydrofuran (2 ml) and water (2 ml) were added to the reaction bottle (100 ml). The resulting mixture was stirred at 0° C. Propionyl chloride (0.03 g, 0.32 mmol) was dropped into the reaction mixture. And then the mixture was stirred for a further 0.5 h. Then the mixture was concentrated and dissolved in ethyl acetate (50 ml). The organic layers were combined, washed with brine (50 ml*3), and then concentrated under reduced pressure. The residue was purified by preparative thin layer chromatography (dichloromethane/methanol=20/1) to give the compound 518 (8.1 mg, yield 9.1%). MS-ESI (M+H)⁺: 527.8. ¹H NMR (500 MHz, DMSO-d₆) δ 11.89 (s, 1H), 11.40 (s, 1H), 9.07 (s, 1H), 8.42 (d, J=8.1 Hz, 1H), 8.29 (s, 1H), 8.16 (d, J=3.0 Hz, 1H), 7.80 (dd, J=8.7, 2.5 Hz, 1H), 7.26-7.11 (m, 2H), 7.07 (t, J=7.9 Hz, 1H), 6.88-6.70 (m, 2H), 3.96 (s, 3H), 2.66 (s, 4H), 2.59 (s, 3H), 1.23 (s, 6H), 1.12 (t, J=7.6 Hz, 3H), 0.90-0.77 (m, 2H).

### Procedure for the Synthesis of 520, 521 and 522

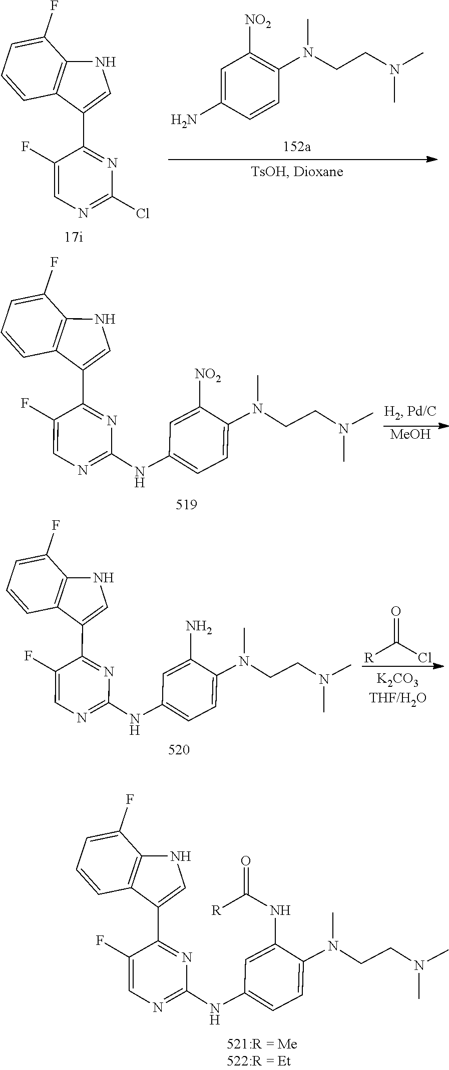

### N¹-(2-(dimethylamino)ethyl)-N⁴-(5-fluoro-4-(7-fluoro-1H-indol-3-yl)pyrimidin-2-yl)-N-methyl-2-nitrobenzene-1,4-diamine (519)

A mixture of 17i (400 mg, 1.51 mmol), 152a (466 mg, 1.96 mmol) and TsOH (780 mg, 4.52 mmol) in dioxane (30 mL) was stirred at 80° C. for 3 days. The reaction mixture was then cooled to room temperature, filtered through celite and concentrated under reduced pressure to give a residue. The residue was purified by column (dichloromethane/methanol=25/1-15/1) to afford 519 (405 mg, yield: 57.5%) as a red solid. LC-MS (ESI): m/z 467.5 [M+H]⁺.

### N¹-(2-(dimethylamino)ethyl)-N⁴-(5-fluoro-4-(7-fluoro-1H-indol-3-yl)pyrimidin-2-yl)-N-methylbenzene-1,2,4-triamine (520)

Pd/C (5% w/w, 50 mg) was added into a solution of 519 (405 mg, 0.87 mmol) in methanol (30 mL) at 20° C. Then reaction was replaced with H₂ for 3 times and then stirred overnight. The reaction was filtered through celite and concentrated under reduced pressure to give a residue. The residue was purified by column (dichloromethane/methanol=25/1-20/1) to afford 520 (400 mg) as a brown oil. LC-MS (ESI): m/z 438.7 [M+H]^{+ 1}H NMR (500 MHz, DMSO) δ 12.52 (s, 1H), 9.20 (s, 1H), 8.58 (d, J=7.8 Hz, 1H), 8.39 (d, J=3.7 Hz, 1H), 8.17 (s, 1H), 7.16-7.05 (m, 3H), 6.94 (d, J=8.5 Hz, 1H), 6.89 (dd, J=8.5, 2.3 Hz, 1H), 3.20-3.15 (m, 2H), 3.14-3.09 (m, 2H), 2.72 (s, 6H), 2.52 (s, 3H).

### N-(2-((2-(dimethylamino)ethyl)(methyl)amino)-5-((5-fluoro-4-(7-fluoro-1H-indol-3-yl)pyrimidin-2-yl)amino)phenyl)acetamide (521)

CH₃COCl (100 mg, 1.28 mmol) was added into a solution of 520 (150 mg, 0.18 mmol) in tetrahydrofuran/H₂O (4 mL/2 mL) with K₂CO₃ (200 mg, 1.45 mmol) at 0-10° C. Then reaction was stirred for 0.5 h. The reaction was concentrated under reduced pressure to give a residue. The residue was purified by preparative thin layer chromatography (dichloromethane/methanol=10/1) to afford 521 (21 mg, yield: 24.0% over 2 steps) as a yellow solid. LC-MS (ESI): m/z 480.0 [M+H]⁺. ¹H NMR (500 MHz, DMSO) δ 12.52 (s, 1H), 9.51 (s, 1H), 8.55 (d, J=7.5 Hz, 1H), 8.43 (d, J=3.7 Hz, 1H), 8.37 (s, 1H), 8.19 (t, J=2.7 Hz, 1H), 7.53 (dd, J=8.7, 2.2 Hz, 1H), 7.23 (d, J=8.7 Hz, 1H), 7.18-7.03 (m, 2H), 3.35-3.29 (m, 2H), 3.09-2.98 (m, 2H), 2.62 (s, 3H), 2.52 (s, 6H), 2.14 (s, 3H).

### N-(2-((2-(dimethylamino)ethyl)(methyl)amino)-5-((5-fluoro-4-(7-fluoro-1H-indol-3-yl)pyrimidin-2-yl)amino)phenyl)propionamide (522)

CH₃CH₂COCl (100 mg, 1.08 mmol) was added into a solution of 520 (120 mg, 0.27 mmol) in tetrahydrofuran/H₂O (4 mL/2 mL) with K₂CO₃ (200 mg, 1.45 mmol) at 0-10° C. Then reaction was stirred for 0.5 h. The reaction was concentrated under reduced pressure to give a residue. The residue was purified by preparative thin layer chromatography (dichloromethane/methanol=10/1) to afford 522 (24 mg, yield: 18.3% over 2 steps) as a yellow solid. LC-MS (ESI): m/z 494.5 [M+H]^{+ 1}H NMR (500 MHz, DMSO) δ 12.52 (s, 1H), 9.49 (s, 1H), 8.55 (d, J=7.5 Hz, 1H), 8.43 (d, J=3.7 Hz, 1H), 8.40 (s, 1H), 8.19 (t, J=2.6 Hz, 1H), 7.52 (dd, J=8.7, 2.3 Hz, 1H), 7.24 (d, J=8.7 Hz, 1H), 7.17-7.03 (m, 2H), 3.07-2.85 (m, 2H), 2.63 (s, 3H), 2.52 (s, 6H), 2.46-2.41 (m, 2H), 2.41-2.36 (m, 2H), 1.10 (t, J=7.5 Hz, 3H).

### Procedure for the Synthesis of 524, 525 and 526

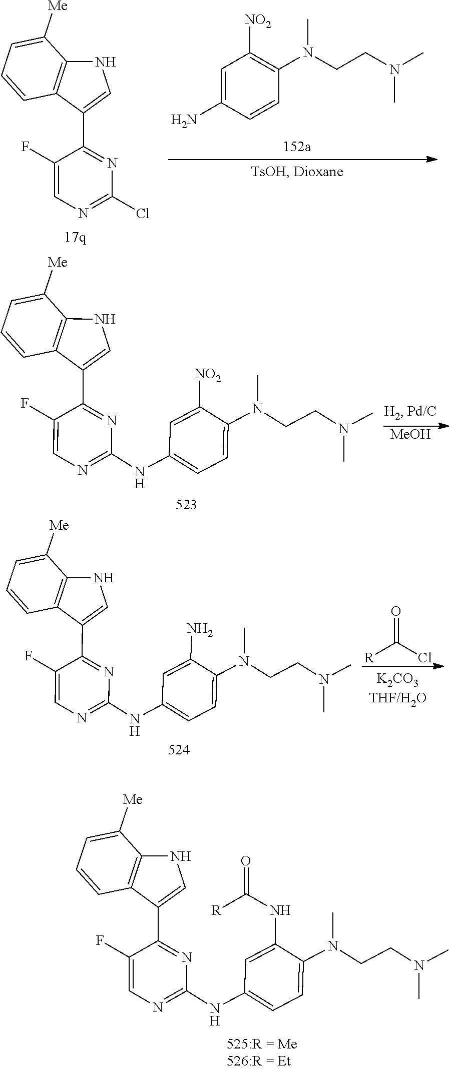

### N¹-(2-(dimethylamino)ethyl)-N⁴-(5-fluoro-4-(7-methyl-1H-indol-3-yl)pyrimidin-2-yl)-N-methyl-2-nitrobenzene-1,4-diamine (523)

A mixture of 17q (400 mg, 1.53 mmol), 152a (475 mg, 1.99 mmol) and TsOH (790 mg, 4.59 mmol) in dioxane (30 mL) was stirred at 80° C. for 3 days. The reaction mixture was then cooled to room temperature, filtered through celite and concentrated under reduced pressure to give a residue. The residue was purified by column (dichloromethane/methanol=25/1-15/1) to afford 523 (350 mg, yield: 49.4%) as a gray solid. LC-MS (ESI): m/z 464.5 [M+H]⁺.

### N¹-(2-(dimethylamino)ethyl)-N⁴-(5-fluoro-4-(7-methyl-1H-indol-3-yl)pyrimidin-2-yl)-N¹-methylbenzene-1,2,4-triamine (524)

Pd/C (5% w/w, 50 mg) was added into a solution of 523 (350 mg, 0.76 mmol) in methanol (30 mL) at 20° C. Then reaction was replaced with H₂ for 3 times and then stirred overnight. The reaction was filtered through celite and concentrated under reduced pressure to give a residue. The residue was purified by column (dichloromethane/methanol=25/1-20/1) to afford 524 (360 mg) as an orange solid. LC-MS (ESI): m/z 434.8 [M+H]⁺. ¹H NMR (500 MHz, DMSO) δ 12.01 (d, J=2.3 Hz, 1H), 9.15 (s, 1H), 8.61 (d, J=7.8 Hz, 1H), 8.35 (d, J=3.9 Hz, 1H), 8.11 (t, J=3.0 Hz, 1H), 7.10-7.00 (m, 3H), 6.95-6.88 (m, 2H), 3.23-3.16 (m, 3H), 3.15-3.10 (m, 2H), 2.73 (s, 6H), 2.53 (s, 3H), 2.52 (s, 3H).

### N-(2-((2-(dimethylamino)ethyl)(methyl)amino)-5-((5-fluoro-4-(7-methyl-1H-indol-3-yl)pyrimidin-2-yl)amino)phenyl)acetamide (525)

CH₃COCl (100 mg, 1.28 mmol) was added into a solution of 524 (150 mg, 0.35 mmol) in tetrahydrofuran/H₂O (4 mL/2 mL) with K₂CO₃ (200 mg, 1.45 mmol) at 0-10° C. Then reaction was stirred for 0.5 h. The reaction was concentrated under reduced pressure to give a residue. The residue was purified by preparative thin layer chromatography (dichloromethane/methanol=10/1) to afford 525 (40 mg, yield: 24.3% over 2 steps) as a yellow solid. LC-MS (ESI): m/z 476.6 [M+H]^{+ 1}H NMR (500 MHz, DMSO) δ 12.00 (d, J=2.4 Hz, 1H), 9.60 (s, 1H), 9.45 (s, 1H), 8.57 (d, J=7.9 Hz, 1H), 8.37 (d, J=3.9 Hz, 1H), 8.33 (s, 1H), 8.12 (t, J=3.0 Hz, 1H), 7.53 (dd, J=8.7, 2.5 Hz, 1H), 7.18 (d, J=8.7 Hz, 1H), 7.10-7.06 (m, 1H), 7.03 (d, J=7.0 Hz, 1H), 3.21-3.11 (m, J=4.5 Hz, 4H), 2.68 (s, 6H), 2.57 (s, 3H), 2.52 (s, 3H), 2.20 (s, 3H).

### N-(2-((2-(dimethylamino)ethyl)(methyl)amino)-5-((5-fluoro-4-(7-methyl-1H-indol-3-yl)pyrimidin-2-yl)amino)phenyl)propionamide (526)

CH₃CH₂COCl (100 mg, 1.08 mmol) was added into a solution of 524 (150 mg, 0.35 mmol) in tetrahydrofuran/H₂O (4 mL/2 mL) with K₂CO₃ (200 mg, 1.45 mmol) at 0-10° C. Then reaction was stirred for 0.5 h. The reaction was concentrated under reduced pressure to give a residue. The residue was purified by preparative thin layer chromatography (dichloromethane/methanol=10/1) to afford 526 (41 mg, yield: 24.2% over 2 steps) as a yellow solid. LC-MS (ESI): m/z 490.8 [M+H]^{+ 1}H NMR (500 MHz, DMSO) δ 11.98 (d, J=2.4 Hz, 1H), 9.62 (s, 1H), 9.44 (s, 1H), 8.57 (d, J=7.8 Hz, 1H), 8.38 (d, J=3.9 Hz, 1H), 8.35 (s, 1H), 8.12 (t, J=3.0 Hz, 1H), 7.53 (dd, J=8.7, 2.5 Hz, 1H), 7.20 (d, J=8.7 Hz, 1H), 7.09-7.01 (m, 2H), 3.13-3.00 (m, 2H), 2.59 (s, 3H), 2.57-2.53 (m, 2H), 2.52 (s, 3H), 2.47-2.41 (m, 2H), 1.09 (t, J=7.6 Hz, 3H).

### Procedure for the Synthesis of 528, 529 and 530

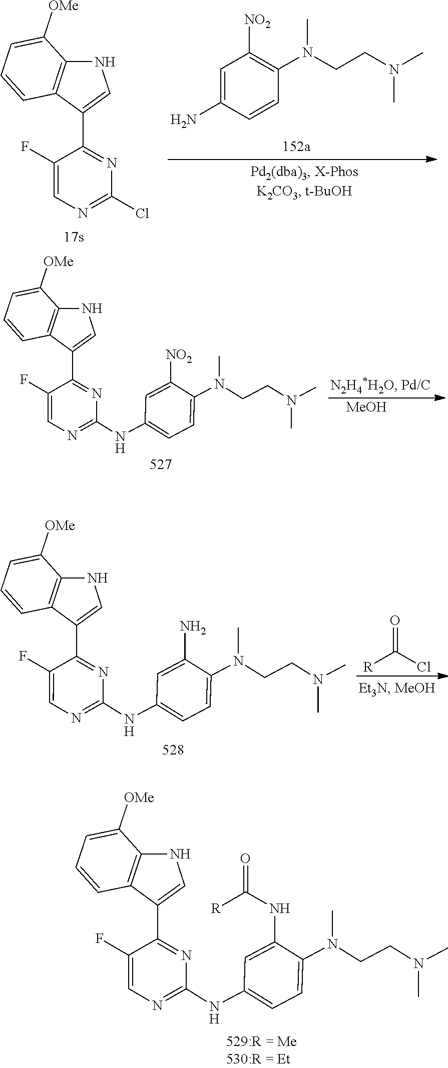

### N¹-(2-(dimethylamino)ethyl)-N¹-methyl-N⁴-(5-fluoro-4-(7-methoxy-1H-indol-3-yl) pyrimidin-2-yl)-2-nitrobenzene-1,4-diamine (527)

A mixture of 17s (1.20 g, 4.32 mmol), 152a (0.94 g, 3.93 mmol), Pd₂(dba)₃ (0.74 g, 0.81 mmol), X-Phos (0.72 g, 1.51 mmol) and K₂CO₃ (1.87 g 13.55 mmol) in t-BuOH (50 mL) was stirred at 90° C. overnight. After cooling to 45° C., the mixture was filtered off and the filtrate was concentrated under reduced pressure. The residue was dissolved in ethyl acetate (60 mL), and washed with H₂O (20 mL×2), dried with Na₂SO₄. After concentration, the residue was purified by silica gel column chromatography (dichloromethane/methanol=20/1) to give the product 527 (0.29 g, yield: 15.4%) as a red solid.

### N¹-(2-(dimethylamino)ethyl)-N¹-methyl-N⁴-(5-fluoro-4-(7-methoxy-1H-indol-3-yl) pyrimidin-2-yl)benzene-1,2,4-triamine (528)

To a solution of 527 (0.29 g, 0.60 mmol) in methanol (5 mL), 5% Pd/C (0.04 g, 17 μmol) and hydrazine hydrate (5 mL) were added. The mixture was stirred at 50° C. for 3 h. The mixture was filtered off and the filtrate was concentrated under reduced pressure. The residue diluted with ethyl acetate (25 mL). The organic phase was washed with H₂O (10 mL×2), dried with Na₂SO₄. The organic phase was concentrated to give the crude product 528 (0.18 g, yield: 66.2%) as a brown oil. The above crude product (0.03 g) was further purified by preparative thin layer chromatography using preparative thin layer chromatography (dichloromethane/methanol=10/1) to give the refined product 528 (8.7 mg) as a gray solid. MS-ESI (M+H)⁺: 450.5. ¹H NMR (500 MHz, DMSO-d₆) δ 12.11 (s, 1H), 9.15 (s, 1H), 8.35 (d, J=2.4 Hz, 1H), 8.34 (s, 1H), 8.04-7.97 (m, 1H), 7.13-7.06 (m, 2H), 6.93 (d, J=8.5 Hz, 1H), 6.92-6.89 (m, 1H), 6.81 (d, J=7.8 Hz, 1H), 3.96 (s, 3H), 3.20 (s, 2H), 3.13 (d, J=5.2 Hz, 2H), 2.74 (s, 6H), 2.52 (s, 3H).

### N-(2-((2-(dimethylamino)ethyl)(methyl)amino)-5-((5-fluoro-4-(7-methoxy-1H-indol-3-yl)pyrimidin-2-yl)amino)phenyl)acetamide (529)

A solution of 528 (0.07 g, 0.16 mmol) and Et₃N (0.03 g, 0.31 mmol) in methanol (2 mL) was stirred at 0° C. for 15 min. Then acetyl chloride (0.02 g, 0.25 mmol) was added dropwise and the mixture was stirred at 0° C. for 30 min. The reaction mixture was quenched by saturated NaHCO₃ (2 mL) and diluted with dichloromethane (10 mL). The organic phase was washed with H₂O (3 mL×2). After concentration, the residue was purified by preparative thin layer chromatography (dichloromethane/methanol=12/1) to give the title product 529 (11.7 mg, yield: 15.28%) as a light brown solid. MS-ESI (M+H)⁺: 492.7. ¹H NMR (500 MHz, DMSO-d₆) δ 12.12 (s, 1H), 9.47 (s, 1H), 8.38 (d, J=3.8 Hz, 1H), 8.31 (d, J=7.8 Hz, 2H), 8.02 (t, J=3.0 Hz, 1H), 7.55 (dd, J=8.7, 2.3 Hz, 1H), 7.21 (d, J=8.7 Hz, 1H), 7.10 (t, J=7.9 Hz, 1H), 6.82 (d, J=7.8 Hz, 1H), 3.96 (s, 3H), 3.17 (s, 4H), 2.72 (s, 6H), 2.58 (s, 3H), 2.19 (s, 3H).

### N-(2-((2-(dimethylamino)ethyl)(methyl)amino)-5-((5-fluoro-4-(7-methoxy-1H-indol-3-yl)pyrimidin-2-yl)amino)phenyl)propionamide (530)

A solution of 528 (0.07 g, 0.16 mmol) and Et₃N (0.08 g, 0.79 mmol) in methanol (4 mL) was stirred at 0° C. for 12 min. Then propionyl chloride (0.04 g, 0.44 mmol) was added dropwise and the mixture was stirred at 0° C. for 15 min. The reaction mixture was quenched by saturated NaHCO₃ (2 mL) and diluted with dichloromethane (10 mL). The organic phase was washed with H₂O (3 mL×2). After concentration, the residue was purified by preparative thin layer chromatography (dichloromethane/methanol=12/1) to give the title product 530 (18.7 mg, yield: 23.8%) as a white solid. MS-ESI (M+H)⁺: 506.2. ¹H NMR (500 MHz, DMSO-d₆) δ 12.12 (s, 1H), 10.26 (s, 1H), 9.53 (s, 1H), 9.45 (s, 1H), 8.38 (d, J=3.8 Hz, 1H), 8.31 (d, J=7.9 Hz, 2H), 8.01 (t, J=2.7 Hz, 1H), 7.60-7.48 (m, 1H), 7.20 (d, J=8.7 Hz, 1H), 7.10 (t, J=7.9 Hz, 1H), 6.81 (d, J=7.7 Hz, 1H), 3.96 (s, 3H), 3.19 (s, 4H), 2.71 (s, 6H), 2.57 (s, 3H), 2.54 (q, J=8.0 Hz, 2H), 1.09 (t, J=7.5 Hz, 3H).

### Procedure for the Synthesis of 532, 533 and 534

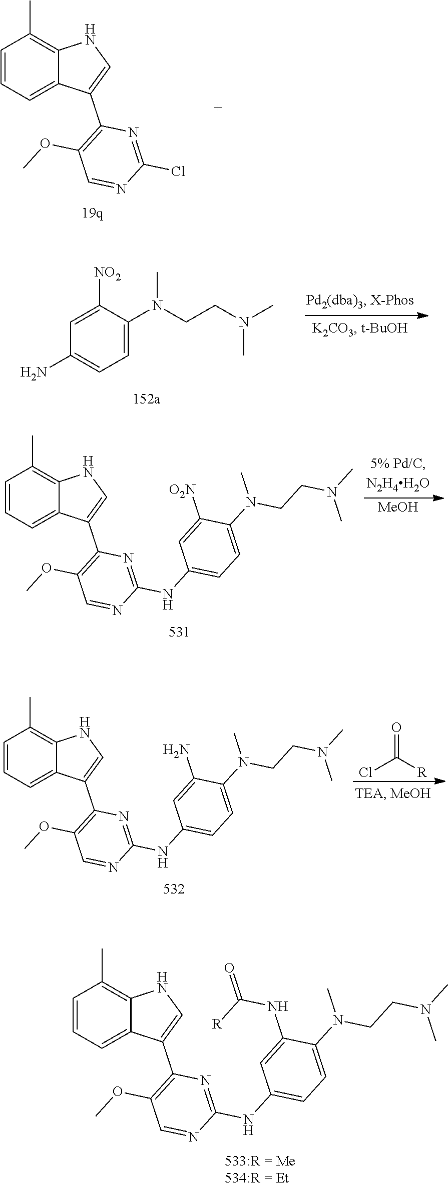

### N¹-(2-(dimethylamino)ethyl)-N¹-methyl-N⁴-(5-methoxy-4-(7-methyl-1H-indol-3-yl) pyrimidin-2-yl)-2-nitrobenzene-1,4-diamine (531)

A mixture of 19q (1.23 g, 4.49 mmol), 152a (0.87 g, 3.65 mmol), Pd₂(dba)₃ (0.75 g, 0.82 mmol), X-Phos (0.76 g, 1.59 mmol) and K₂CO₃ (1.80 g 13.04 mmol) in t-BuOH (50 mL) was stirred at 90° C. overnight. After cooling to 45° C., the mixture was filtered off and the filtrate was concentrated under reduced pressure. The residue was dissolved in ethyl acetate (60 mL), and washed with H₂O (20 mL×2), dried with Na₂SO₄. After concentration, the residue was purified by silica gel column chromatography (dichloromethane/methanol=20/1) to give the product 531 (0.16 g, yield: 9.21%) as a red solid.

### N¹-(2-(dimethylamino)ethyl)-N¹-methyl-N⁴-(5-methoxy-4-(7-methyl-1H-indol-3-yl) pyrimidin-2-yl)benzene-1,2,4-triamine (532)

To a solution of 531 (0.16 g, 0.34 mmol) in methanol (4 mL), 5% Pd/C (0.03 g, 13 mol) and hydrazine hydrate (3 mL) were added. The mixture was stirred at 50° C. for 2 h. The mixture was filtered off and the filtrate was concentrated under reduced pressure. The residue diluted with ethyl acetate (20 mL). The organic phase was washed with H₂O (5 mL×2), dried with Na₂SO₄. The organic phase was concentrated to give the crude product 532 (0.12 g, yield: 80.0%) as a brown oil. The above crude product (0.02 g) was further purified by preparative thin layer chromatography (dichloromethane/methanol=10/1) to give the refined title product 532 (6.2 mg) as a yellow solid. MS-ESI (M+H)⁺: 446.8. ¹H NMR (500 MHz, DMSO-d₆) δ 11.73 (s, 1H), 8.83 (s, 1H), 8.72 (d, J=7.9 Hz, 1H), 8.30 (d, J=2.8 Hz, 1H), 8.16 (s, 1H), 7.13 (d, J=1.7 Hz, 1H), 7.04 (t, J=7.5 Hz, 1H), 7.00 (d, J=7.1 Hz, 1H), 6.95 (dd, J=8.6, 1.9 Hz, 1H), 6.90 (d, J=8.5 Hz, 1H), 3.96 (s, 3H), 3.17 (t, J=4.6 Hz, 2H), 3.13-3.08 (m, 2H), 2.74 (s, 6H), 2.52 (s, 6H).

### N-(2-((2-(dimethylamino)ethyl)(methyl)amino)-5-((5-methoxy-4-(7-methyl-1H-indol-3-yl)pyrimidin-2-yl)amino)phenyl)acetamide (533)

A solution of 532 (0.05 g, 0.11 mmol) and Et₃N (0.02 g, 0.21 mmol) in methanol (2 mL) was stirred at 0° C. for 15 min. Then acetyl chloride (0.02 g, 0.25 mmol) was added dropwise and the mixture was stirred at 0° C. for 30 min. The reaction mixture was quenched by saturated NaHCO₃ (2 mL) and diluted with dichloromethane (10 mL). The organic phase was washed with H₂O (2 mL×2). After concentration, the residue was purified by preparative thin layer chromatography (dichloromethane/methanol=12/1) to give the title product 533 (5.7 mg, yield: 10.4%) as a yellow solid. MS-ESI (M+H)⁺: 488.8. ¹H NMR (500 MHz, DMSO-d₆) δ 11.75 (s, 1H), 9.55 (s, 1H), 9.14 (s, 1H), 8.71 (d, J=7.9 Hz, 1H), 8.35 (s, 1H), 8.32 (d, J=3.0 Hz, 1H), 8.19 (s, 1H), 7.58 (dd, J=8.7, 2.4 Hz, 1H), 7.17 (d, J=8.7 Hz, 1H), 7.05 (t, J=7.5 Hz, 1H), 7.00 (d, J=6.9 Hz, 1H), 3.97 (s, 3H), 3.14 (s, 4H), 2.69 (s, 6H), 2.58 (s, 6H), 2.18 (s, 3H).

### N-(2-((2-(dimethylamino)ethyl)(methyl)amino)-5-((5-methoxy-4-(7-methyl-1H-indol-3-yl)pyrimidin-2-yl)amino)phenyl)propionamide (534)

A solution of 532 (0.05 g, 0.11 mmol) and Et₃N (0.02 g, 0.22 mmol) in methanol (2 mL) was stirred at 0° C. for 20 min. Then propionyl chloride (0.02 g, 0.22 mmol) was added dropwise and the mixture was stirred at 0° C. for 20 min. The reaction mixture was quenched by saturated NaHCO₃ (2 mL) and diluted with dichloromethane (10 mL). The organic phase was washed with H₂O (2 mL×2). After concentration, the residue was purified by preparative thin layer chromatography (dichloromethane/methanol=12/1) to give the title product 534 (11 mg, yield: 19.54%) as a yellow solid. MS-ESI (M+H)⁺: 502.8. ¹H NMR (500 MHz, DMSO-d₆) δ 11.75 (s, 1H), 9.46 (s, 1H), 9.15 (s, 1H), 8.71 (d, J=7.9 Hz, 1H), 8.35-8.31 (m, 1H), 8.30 (s, 1H), 8.20 (s, 1H), 7.60 (dd, J=8.7, 2.4 Hz, 1H), 7.17 (d, J=8.7 Hz, 1H), 7.07-7.02 (m, 1H), 7.00 (d, J=7.0 Hz, 1H), 3.97 (s, 3H), 3.20 (s, 4H), 2.73 (s, 6H), 2.56 (s, 3H), 2.53 (s, 2H), 2.52 (s, 3H), 1.10 (t, J=7.5 Hz, 3H).

### Procedure for the Synthesis of 536, 537 and 538

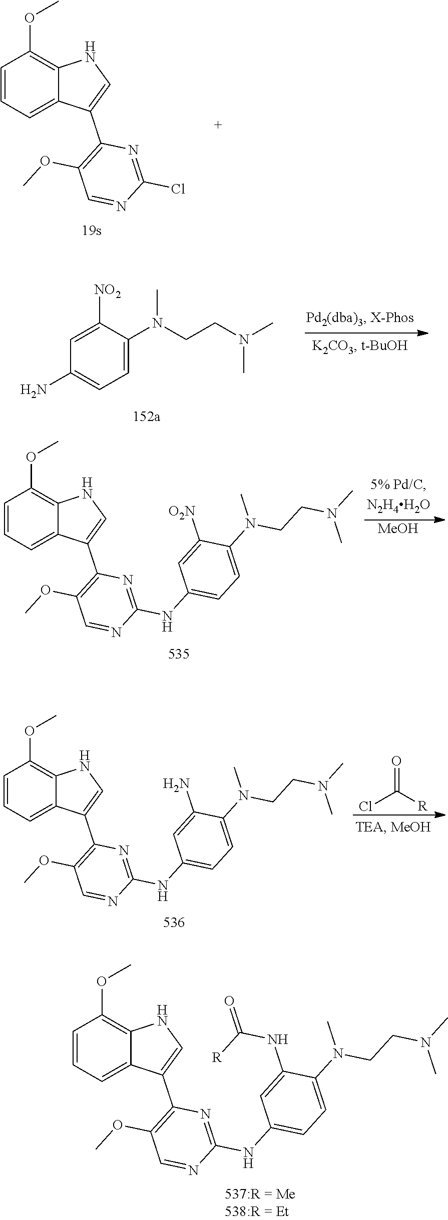

### N¹-(2-(dimethylamino)ethyl)-N¹-methyl-N⁴-(5-methoxy-4-(7-methoxy-1H-indol-3-yl) pyrimidin-2-yl)-2-nitrobenzene-1,4-diamine (535)

A mixture of 19s (2.01 g, 6.94 mmol), 152a (1.12 g, 4.70 mmol), Pd₂(dba)₃ (0.63 g, 0.69 mmol), X-Phos (0.62 g, 1.30 mmol) and K₂CO₃ (2.89 g 20.94 mmol) in t-BuOH (50 mL) was stirred at 90° C. overnight. After cooling to 45° C., the mixture was filtered off and the filtrate was concentrated under reduced pressure. The residue was dissolved in ethyl acetate (60 mL), and washed with H₂O (20 mL×2), dried with Na₂SO₄. After concentration, the residue was purified by silica gel column chromatography (dichloromethane/methanol=20/1) to give the product 535 (0.18 g, yield: 7.92%) as a brown oil.

### N¹-(2-(dimethylamino)ethyl)-N¹-methyl-N⁴-(5-methoxy-4-(7-methoxy-1H-indol-3-yl) pyrimidin-2-yl)benzene-1,2,4-triamine (536)

To a solution of 535 (0.18 g, 0.37 mmol) in methanol (4 mL), 5% Pd/C (0.03 g, 13 μmol) and hydrazine hydrate (3 mL) were added. The mixture was stirred at 50° C. for 2.5 h. The mixture was filtered off and the filtrate was concentrated under reduced pressure. The residue diluted with ethyl acetate (25 mL). The organic phase was washed with H₂O (5 mL×2), dried with Na₂SO₄. The organic phase was concentrated to give the crude product 536 (0.12 g, yield: 71.00%) as a brown oil. The above crude product (0.02 g) was further purified by preparative thin layer chromatography (dichloromethane/methanol=10/1) to give the refined product 536 (3.6 mg) as a yellow solid. MS-ESI (M+H)⁺: 462.8. ¹H NMR (500 MHz, DMSO-d₆) δ 11.88 (s, 1H), 8.82 (s, 1H), 8.47 (d, J=8.2 Hz, 1H), 8.22 (s, 1H), 8.16 (s, 1H), 7.13 (d, J=2.1 Hz, 1H), 7.05 (t, J=7.9 Hz, 1H), 6.95 (dd, J=8.5, 2.1 Hz, 1H), 6.90 (d, J=8.5 Hz, 1H), 6.77 (d, J=7.8 Hz, 1H), 5.76 (s, 1H), 3.95 (s, 6H), 3.17 (d, J=4.3 Hz, 2H), 3.13-3.07 (m, 2H), 2.73 (s, 6H), 2.51 (s, 3H).

### N-(2-((2-(dimethylamino)ethyl)(methyl)amino)-5-((5-methoxy-4-(7-methyl-1H-indol-3-yl)pyrimidin-2-yl)amino)phenyl)acetamide (537)

A solution of 536 (0.05 g, 0.11 mmol) and Et₃N (0.02 g, 0.20 mmol) in methanol (2 mL) was stirred at 0° C. for 20 min. Then acetyl chloride (0.02 g, 0.26 mmol) was added dropwise and the mixture was stirred at 0° C. for 30 min. The reaction mixture was quenched by saturated NaHCO₃ (2 mL) and diluted with dichloromethane (10 mL). The organic phase was washed with H₂O (2 mL×2). After concentration, the residue was purified by preparative thin layer chromatography (dichloromethane/methanol=12/1) to give the title product 537 (4.1 mg, yield: 7.52%) as a yellow solid. MS-ESI (M+H)⁺: 504.3. ¹H NMR (500 MHz, DMSO-d₆) δ 11.89 (s, 1H), 9.45 (s, 1H), 9.16 (s, 1H), 8.45 (d, J=8.1 Hz, 1H), 8.30 (s, 1H), 8.24 (d, J=3.0 Hz, 1H), 8.19 (s, 1H), 7.60 (dd, J=8.7, 2.4 Hz, 1H), 7.21-7.16 (m, 1H), 7.06 (t, J=7.9 Hz, 1H), 6.81-6.74 (m, 1H), 3.96 (d, J=5.3 Hz, 6H), 3.18 (s, 4H), 2.75 (s, 6H), 2.57 (s, 3H), 2.18 (s, 3H).

### N-(2-((2-(dimethylamino)ethyl)(methyl)amino)-5-((5-methoxy-4-(7-methoxy-1H-indol-3-yl)pyrimidin-2-yl)amino)phenyl)propionamide (538)

A solution of 536 (0.05 g, 0.11 mmol) and Et₃N (0.02 g, 0.22 mmol) in methanol (2 mL) was stirred at 0° C. for 20 min. Then propionyl chloride (0.02 g, 0.22 mmol) was added dropwise and the mixture was stirred at 0° C. for 30 min. The reaction mixture was quenched by saturated NaHCO₃ (2 mL) and diluted with dichloromethane (10 mL). The organic phase was washed with H₂O (2 mL×2). After concentration, the residue was purified by preparative thin layer chromatography (dichloromethane/methanol=12/1) to give the title product 538 (2.0 mg, yield: 3.57%) as a brown solid. MS-ESI (M+H)⁺: 518.8. ¹H NMR (500 MHz, DMSO-d₆) δ 11.89 (s, 1H), 9.15 (s, 1H), 8.45 (d, J=8.2 Hz, 1H), 8.30 (s, 1H), 8.23 (d, J=3.0 Hz, 1H), 8.20 (s, 1H), 7.60 (dd, J=8.7, 2.3 Hz, 1H), 7.18 (d, J=8.7 Hz, 1H), 7.06 (t, J=7.9 Hz, 1H), 6.78 (d, J=7.7 Hz, 1H), 3.96 (d, J=6.7 Hz, 6H), 3.19 (s, 2H), 2.74 (m, 5H), 2.57 (s, 3H), 2.52 (s, 6H), 1.10 (t, J=7.6 Hz, 3H).

### Procedure for the Synthesis of 540

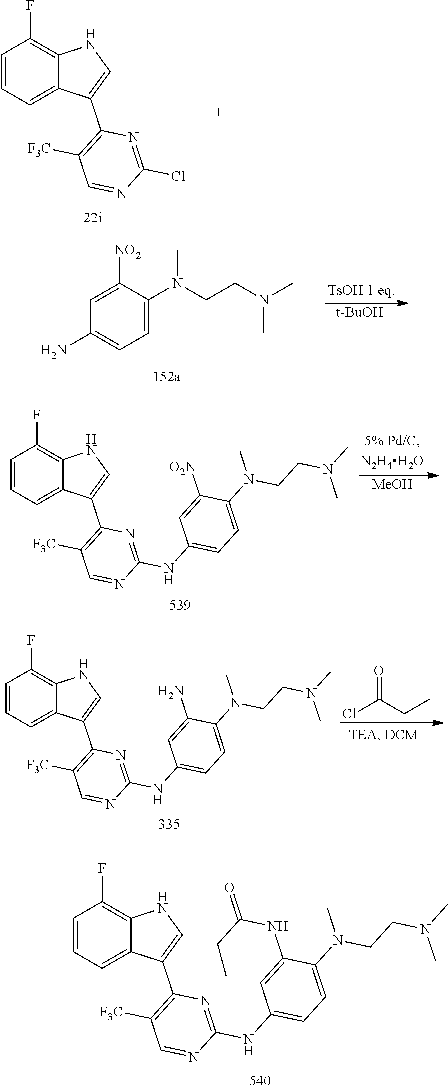

### N¹-(2-(dimethylamino)ethyl)-N¹-methyl-N⁴-(4-(7-fluoro-1H-indol-3-yl)-5-(trifluoromethyl)pyrimidin-2-yl)-2-nitrobenzene-1,4-diamine (539)

A mixture of 22i (0.14 g, 0.44 mmol), 152a (0.12 g, 0.50 mmol) and TsOH (0.11 g, 0.64 mmol) in t-BuOH (10 mL) was stirred at 100° C. overnight. After cooling to room temperature, the mixture was neutralized with saturated NaHCO₃ and concentrated under reduced pressure. The residue was dissolved in dichloromethane (30 mL), and washed with H₂O (10 mL×2), dried with Na₂SO₄. After concentration, the residue was purified by silica gel column chromatography (dichloromethane/methanol=15/1) to give the desired product 539 (0.25 g, yield: 95.8%) as a red oil.

### N¹-(2-(dimethylamino)ethyl)-N¹-methyl-N⁴-(4-(7-fluoro-1H-indol-3-yl)-5-(trifluoromethyl)pyrimidin-2-yl)benzene-1,2,4-triamine (335)

To a solution of 539 (0.25 g, 0.48 mmol) in methanol (5 mL), 5% Pd/C (0.04 g, 0.02 mmol) and hydrazine hydrate (3 mL) were added. The mixture was stirred at 60° C. for 3.5 h. The mixture was filtered off and the filtrate was concentrated under reduced pressure. The residue diluted with dichloromethane (20 mL). The organic phase was washed with H₂O (10 mL×2), dried with Na₂SO₄. After concentration, the residue was purified by silica gel column chromatography (dichloromethane/methanol=15/1) to give the product 335 (0.17 g, yield: 72.2%) as a light brown solid.

### N-(2-((2-(dimethylamino)ethyl)(methyl)amino)-5-((4-(7-fluoro-1H-indol-3-yl)-5-(trifluoromethyl)pyrimidin-2-yl)amino)phenyl)propionamide (540)

A solution of 335 (0.17 g, 0.35 mmol) and Et₃N (0.07 g, 0.70 mmol) in dichloromethane (3 mL) was stirred at 0° C. for 15 min. Then propionyl chloride (0.09 g, 0.92 mmol) was added dropwise and the mixture was stirred at 0° C. for 10 min. The reaction mixture was quenched by saturated NaHCO₃ (3 mL) and diluted with dichloromethane (20 mL). The organic phase was washed with H₂O (10 mL×2). After concentration, the residue was purified by preparative thin layer chromatography (dichloromethane/methanol=12/1) to give the title product 540 (42 mg, yield: 22.2%) as a yellow solid. MS-ESI (M+H)⁺: 543.8. ¹H NMR (500 MHz, DMSO-d₆) δ 12.45 (s, 1H), 10.06 (s, 1H), 9.63 (s, 1H), 8.74 (s, 1H), 8.34 (s, 1H), 8.10 (s, 1H), 7.86 (s, 1H), 7.54 (d, J=7.4 Hz, 1H), 7.29-7.12 (m, 1H), 7.12-6.93 (m, 2H), 3.10 (s, 4H), 2.98 (s, 2H), 2.58 (s, 5H), 2.51 (s, 6H), 1.14-1.00 (t, 3H).

### Procedure for the Synthesis of 542

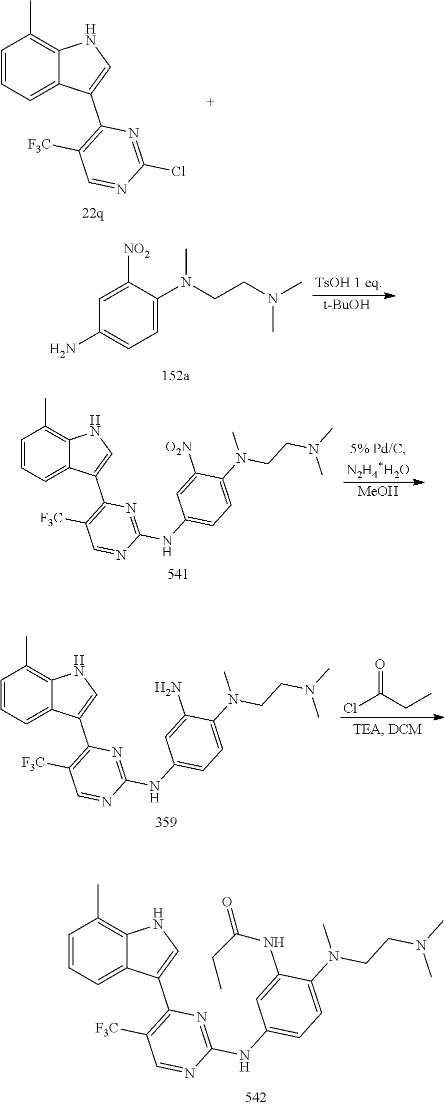

### N¹-(2-(dimethylamino)ethyl)-N¹-methyl-N⁴-(4-(7-methyl-1H-indol-3-yl)-5-(trifluoromethyl)pyrimidin-2-yl)-2-nitrobenzene-1,4-diamine (541)

A mixture of 22q (0.61 g, 1.96 mmol), 152a (0.44 g, 1.86 mmol) and TsOH (0.34 g, 1.98 mmol) in t-BuOH (25 mL) was stirred at 100° C. overnight. After cooling to room temperature, the mixture was neutralized with saturated NaHCO₃ and concentrated under reduced pressure. The residue was dissolved in dichloromethane (50 mL), and washed with H₂O (10 mL×2), dried with Na₂SO₄. After concentration, the residue was purified by silica gel column chromatography (dichloromethane/methanol=15/1) to give the desired product 541 (0.86 g, yield: 89.8%) as a red solid.

### N¹-(2-(dimethylamino)ethyl)-N¹-methyl-N⁴-(4-(7-methyl-1H-indol-3-yl)-5-(trifluoromethyl)pyrimidin-2-yl)benzene-1,2,4-triamine (359)

To a solution of 541 (0.86 g, 1.67 mmol) in methanol (10 mL), 5% Pd/C (0.19 g, 0.09 mmol) and hydrazine hydrate (10 mL) were added. The mixture was stirred at 60° C. for 2.5 h. The mixture was filtered off and the filtrate was concentrated under reduced pressure. The residue diluted with dichloromethane (50 mL). The organic phase was washed with H₂O (20 mL×2), dried with Na₂SO₄. After concentration, the residue was purified by silica gel column chromatography (dichloromethane/methanol=15/1) to give the product 359 (0.75 g, yield: 92.6%) as a light brown solid.

### N-(2-((2-(dimethylamino)ethyl)(methyl)amino)-5-((4-(7-methyl-1H-indol-3-yl)-5-(trifluoromethyl)pyrimidin-2-yl)amino)phenyl)propionamide (542)

A solution of 359 (0.22 g, 0.45 mmol) and Et₃N (0.09 g, 0.88 mmol) in dichloromethane (10 mL) was stirred at 0° C. for 10 min. Then propionyl chloride (0.08 g, 0.86 mmol) was added dropwise and the mixture was stirred at 0° C. for 15 min. The reaction mixture was quenched by saturated NaHCO₃ (5 mL) and diluted with dichloromethane (20 mL). The organic phase was washed with H₂O (10 mL×2). After concentration, the residue was purified by silica gel column chromatography (dichloromethane/methanol=15/1) to give the product 542 (0.18 g, yield: 73.3%) as a gray solid. MS-ESI (M+H)⁺: 541.1. ¹H NMR (500 MHz, DMSO-d₆) δ 11.85 (s, 1H), 9.97 (s, 1H), 9.91 (s, 1H), 8.71 (s, 1H), 8.45 (s, 1H), 8.15 (s, 1H), 7.82 (d, J=2.5 Hz, 1H), 7.53 (dd, J=8.6, 2.5 Hz, 1H), 7.21 (d, J=8.6 Hz, 1H), 7.06-6.94 (m, 2H), 2.81 (t, J=5.7 Hz, 2H), 2.65 (s, 3H), 2.51 (s, 3H), 2.34 (q, J=7.5 Hz, 2H), 2.28 (t, J=5.7 Hz, 2H), 2.21 (s, 6H), 1.10 (t, J=7.5 Hz, 3H).

### Procedure for the Synthesis of 543

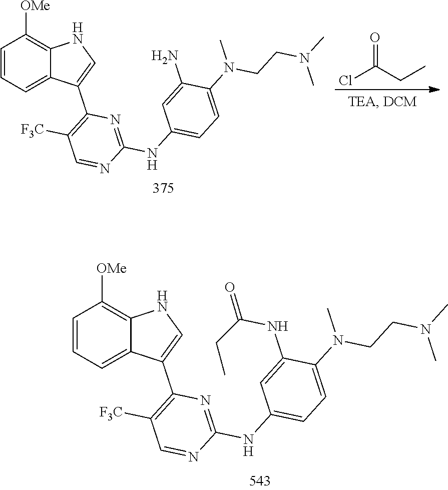

### N-(2-((2-(dimethylamino)ethyl)(methyl)amino)-5-((4-(7-methoxy-1H-indol-3-yl)-5-(trifluoromethyl)pyrimidin-2-yl)amino)phenyl)propionamide (543)

A solution of 375 (0.11 g, 0.22 mmol) and Et₃N (0.08 g, 0.79 mmol) in dichloromethane (10 mL) was stirred at 0° C. for 15 min. Then propionyl chloride (0.04 g, 0.44 mmol) was added dropwise and the mixture was stirred at 0° C. for another 2.5 h. The reaction mixture was quenched by saturated NaHCO₃ (10 mL) and diluted with dichloromethane (10 mL). The organic phase was washed with H₂O (10 mL×2). After concentration, the residue was purified by preparative thin layer chromatography (dichloromethane/methanol=12/1) to give the title product 543 (44 mg, yield: 35.96%) as a gray solid. MS-ESI (M+H)⁺: 556.9. ¹H NMR (500 MHz, DMSO-d₆) δ 12.03 (s, 1H), 9.99 (s, 1H), 9.70 (s, 1H), 8.70 (s, 1H), 8.36 (s, 1H), 7.91 (s, 1H), 7.73 (s, 1H), 7.56 (dd, J=8.6, 2.3 Hz, 1H), 7.19 (d, J=8.6 Hz, 1H), 7.09-6.98 (m, 1H), 6.78 (d, J=7.7 Hz, 1H), 3.95 (s, 3H), 3.02 (s, 2H), 2.77 (s, 2H), 2.60 (s, 3H), 2.47 (m, J=7.6 Hz, 8H), 1.10 (t, J=7.5 Hz, 3H).

### Procedure for the Synthesis of 545, 546 and 547

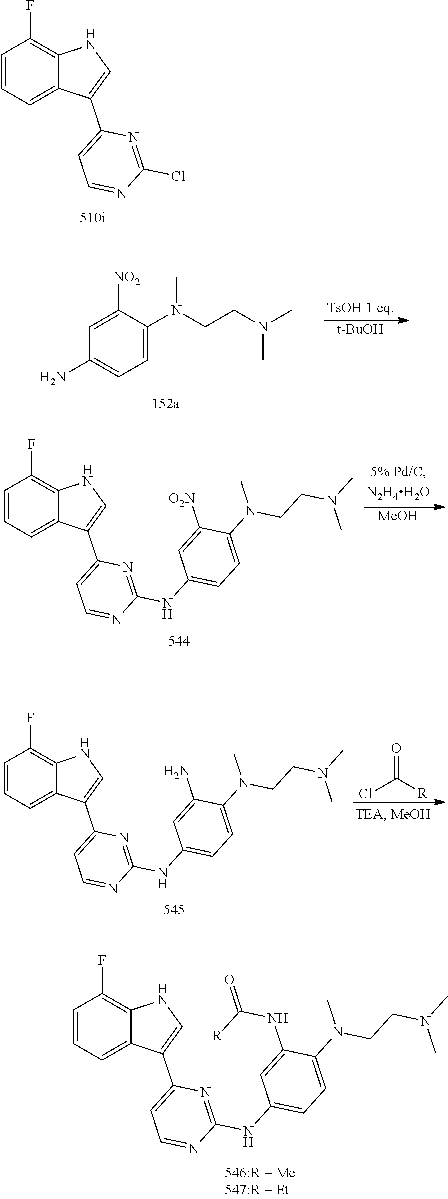

### N¹-(2-(dimethylamino)ethyl)-N¹-methyl-N⁴-(4-(7-methyl-1H-indol-3-yl) pyrimidin-2-yl)-2-nitrobenzene-1,4-diamine (544)

A mixture of 510i (0.75 g, 3.03 mmol), 152a (0.69 g, 2.88 mmol) and TsOH (0.63 g, 3.63 mmol) in t-BuOH (25 mL) was stirred at 90° C. overnight. After cooling to room temperature, the mixture was neutralized with saturated NaHCO₃ and concentrated under reduced pressure. The residue was dissolved in dichloromethane (50 mL), and washed with H₂O (20 mL×2), dried with Na₂SO₄. After concentration, the residue was purified by silica gel column chromatography (dichloromethane/methanol=20/1) to give the product 544 (0.60 g, yield: 46.7%) as a red solid.

### N¹-(2-(dimethylamino)ethyl)-N¹-methyl-N⁴-(4-(7-fluoro-1H-indol-3-yl) pyrimidin-2-yl)benzene-1,2,4-triamine (545)

To a solution of 544 (0.50 g, 1.11 mmol) in methanol (15 mL), 5% Pd/C (0.10 g, 0.05 mmol) and hydrazine hydrate (5 mL) were added. The mixture was stirred at 50° C. for 3 h. The mixture was filtered off and the filtrate was concentrated under reduced pressure. The residue diluted with dichloromethane (25 mL). The organic phase was washed with H₂O (10 mL×2), dried with Na₂SO₄. The organic phase was concentrated to give the crude product 545 (0.45 g, yield: 96.4%) as a brown solid. The above crude (0.06 g) was further purified by preparative thin layer chromatography (dichloromethane/methanol=10/1) to give the refined title product 545 (33 mg) as a light yellow solid. MS-ESI (M+H)⁺: 420.9. ¹H NMR (500 MHz, DMSO-d₆) δ 12.32 (s, 1H), 9.08 (s, 1H), 8.47 (d, J=7.8 Hz, 1H), 8.36 (d, J=2.7 Hz, 1H), 8.29 (d, J=5.3 Hz, 1H), 7.25 (d, J=5.3 Hz, 1H), 7.14 (s, 1H), 7.10 (td, J=7.9, 5.2 Hz, 1H), 7.03 (dd, J=11.2, 7.9 Hz, 1H), 6.93 (s, 2H), 3.21-3.08 (m, 4H), 2.70 (s, 6H), 2.52 (s, 3H).

### N-(2-((2-(dimethylamino)ethyl)(methyl)amino)-5-((4-(7-fluoro-1H-indol-3-yl)pyrimidin-2-yl)amino)phenyl)acetamide (546)

A solution of 545 (0.15 g, 0.36 mmol) and Et₃N (0.05 g, 0.50 mmol) in methanol (2 mL) was stirred at 0° C. for 20 min. Then acetyl chloride (0.03 g, 0.43 mmol) was added dropwise and the mixture was stirred at 0° C. for 30 min. The reaction mixture was quenched by saturated NaHCO₃ (5 mL) and diluted with dichloromethane (20 mL). The organic phase was washed with H₂O (5 mL×2). After concentration, the residue was purified by preparative thin layer chromatography (dichloromethane/methanol=12/1) to give the title product 546 (62 mg, yield: 37.6%) as a gray solid. MS-ESI (M+H)⁺: 462.6. ¹H NMR (500 MHz, DMSO-d₆) δ 12.31 (s, 1H), 9.81 (s, 1H), 9.38 (s, 1H), 8.51 (s, 1H), 8.44-8.39 (m, 2H), 8.32 (d, J=5.3 Hz, 1H), 7.52 (d, J=10.0 Hz, 1H), 7.29 (d, J=5.3 Hz, 1H), 7.21 (d, J=8.7 Hz, 1H), 7.10 (td, J=7.9, 5.1 Hz, 1H), 7.04 (dd, J=11.2, 7.8 Hz, 1H), 3.00 (s, 2H), 2.74 (s, 2H), 2.61 (s, 3H), 2.47 (s, 6H), 2.15 (s, 3H).

### N-(2-((2-(dimethylamino)ethyl)(methyl)amino)-5-((4-(7-fluoro-1H-indol-3-yl)pyrimidin-2-yl)amino)phenyl)propionamide (547)

A solution of 545 (0.16 g, 0.38 mmol) and Et₃N (0.06 g, 0.61 mmol) in methanol (5 mL) was stirred at 0° C. for 20 min. Then propionyl chloride (0.05 g, 0.53 mmol) was added dropwise and the mixture was stirred at 0° C. for 30 min. The reaction mixture was quenched by saturated NaHCO₃ (2 mL) and diluted with dichloromethane (10 mL). The organic phase was washed with H₂O (5 mL×2). After concentration, the residue was purified by preparative thin layer chromatography (dichloromethane/methanol=12/1) to give the title product 547 (26 mg, yield: 14.3%) as a light yellow solid. MS-ESI (M+H)⁺: 476.5. ¹H NMR (500 MHz, DMSO-d₆) δ 12.31 (s, 1H), 9.75 (s, 1H), 9.37 (s, 1H), 8.51 (s, 1H), 8.46-8.38 (m, 2H), 8.33 (d, J=5.3 Hz, 1H), 7.55-7.49 (m, 1H), 7.29 (d, J=5.3 Hz, 1H), 7.22 (d, J=8.7 Hz, 1H), 7.10 (td, J=7.9, 5.2 Hz, 1H), 7.03 (dd, J=11.2, 7.8 Hz, 1H), 2.99 (s, 2H), 2.67 (s, 2H), 2.62 (s, 3H), 2.44 (d, J=6.5 Hz, 8H), 1.11 (t, J=7.5 Hz, 3H).

### Procedure for the Synthesis of 549, 550 and 551

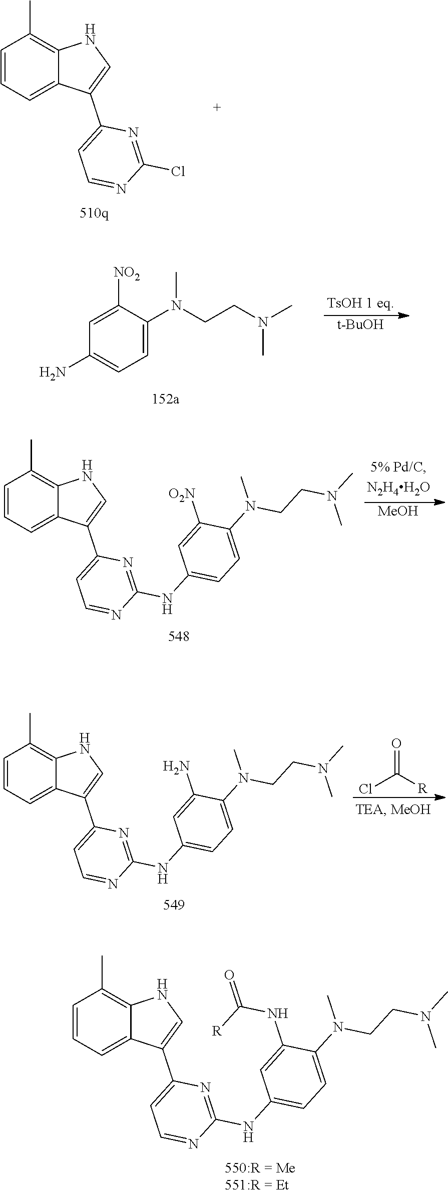

### N¹-(2-(dimethylamino)ethyl)-N¹-methyl-N⁴-(4-(7-methyl-1H-indol-3-yl) pyrimidin-2-yl)-2-nitrobenzene-1,4-diamine (548)

A mixture of 510q (0.50 g, 2.05 mmol), 152a (0.54 g, 2.26 mmol) and TsOH (0.43 g, 2.50 mmol) in t-BuOH (15 mL) was stirred at 100° C. overnight. After cooling to room temperature, the mixture was neutralized with saturated NaHCO₃ and concentrated under reduced pressure. The residue was dissolved in dichloromethane (40 mL), and washed with H₂O (15 mL×2), dried with Na₂SO₄. After concentration, the residue was purified by silica gel column chromatography (dichloromethane/methanol=20/1) to give the desired product 548 (0.50 g, yield: 54.7%) as a red solid.

### N¹-(2-(dimethylamino)ethyl)-N¹-methyl-N⁴-(4-(7-methyl-1H-indol-3-yl) pyrimidin-2-yl)benzene-1,2,4-triamine (549)

To a solution of 548 (0.50 g, 1.12 mmol) in methanol (12 mL), 5% Pd/C (0.10 g, 0.05 mmol) and hydrazine hydrate (5 mL) were added. The mixture was stirred at 50° C. for 3.5 h. The mixture was filtered off and the filtrate was concentrated under reduced pressure. The residue diluted with dichloromethane (20 mL). The organic phase was washed with H₂O (10 mL×2), dried with Na₂SO₄. After concentration, the residue was purified by silica gel column chromatography (dichloromethane/methanol=15/1) to give the crude product 549 (0.36 g, yield: 77.2%) as a light brown solid. The above crude (0.05 g) was further purified by preparative thin layer chromatography (dichloromethane/methanol=10/1) to give the refined title product 549 (23 mg) as a gray solid. MS-ESI (M+H)*: 416.7. ¹H NMR (500 MHz, DMSO-d₆) δ 11.86 (s, 1H), 9.02 (s, 1H), 8.45 (d, J=7.6 Hz, 1H), 8.28 (s, 1H), 8.26 (d, J=5.1 Hz, 1H), 7.23 (d, J=5.0 Hz, 1H), 7.16 (s, 1H), 7.05 (t, J=7.3 Hz, 1H), 6.98 (d, J=6.9 Hz, 1H), 6.96 (d, J=9.0 Hz, 1H), 6.91 (d, J=8.3 Hz, 1H), 3.18-3.08 (m, 4H), 2.68 (s, 6H), 2.51 (s, 6H).

### N-(2-((2-(dimethylamino)ethyl)(methyl)amino)-5-((4-(7-methyl-1H-indol-3-yl)pyrimidin-2-yl)amino)phenyl)propionamide (550)

A solution of 549 (0.15 g, 0.36 mmol) and Et₃N (0.08 g, 0.79 mmol) in methanol (5 mL) was stirred at 0° C. for 5 min. Then propionyl chloride (0.08 g, 0.81 mmol) was added dropwise and the mixture was stirred at 0° C. for 45 min. The reaction mixture was quenched by saturated NaHCO₃ (5 mL) and diluted with dichloromethane (20 mL). The organic phase was washed with H₂O (10 mL×2). After concentration, the residue was purified by preparative thin layer chromatography (dichloromethane/methanol=12/1) to give the title product 550 (91 mg, yield: 53.5%) as a dark yellow solid. MS-ESI (M+H)⁺: 472.1. ¹H NMR (500 MHz, DMSO-d₆) δ 11.78 (s, 1H), 9.74 (s, 1H), 9.31 (s, 1H), 8.50 (s, 1H), 8.41 (d, J=7.3 Hz, 1H), 8.37-8.32 (m, 1H), 8.29 (d, J=5.2 Hz, 1H), 7.60-7.49 (m, 1H), 7.27 (d, J=5.2 Hz, 1H), 7.21 (d, J=8.6 Hz, 1H), 7.04 (t, J=7.4 Hz, 1H), 6.99 (d, J=6.8 Hz, 1H), 3.00 (s, 2H), 2.70 (s, 2H), 2.61 (s, 3H), 2.44 (s, 11H), 1.11 (t, J=7.4 Hz, 3H).

### N-(2-((2-(dimethylamino)ethyl)(methyl)amino)-5-((4-(7-methyl-1H-indol-3-yl)pyrimidin-2-yl)amino)phenyl)acetamide (551)

A solution of 549 (0.15 g, 0.36 mmol) and Et₃N (0.08 g, 0.80 mmol) in methanol (5 mL) was stirred at 0° C. for 10 min. Then acetyl chloride (0.10 g, 1.28 mmol) was added dropwise and the mixture was stirred at 0° C. for 4.5 h. The reaction mixture was quenched by saturated NaHCO₃ (5 mL) and diluted with dichloromethane (15 mL). The organic phase was washed with H₂O (5 mL×2). After concentration, the residue was purified by preparative thin layer chromatography (dichloromethane/methanol=12/1) to give the title product 551 (97 mg, yield: 58.7%) as a light yellow solid. MS-ESI (M+H)⁺: 458.2. ¹H NMR (500 MHz, DMSO-d₆) δ 11.86 (s, 1H), 9.79 (s, 1H), 9.31 (s, 1H), 8.50 (s, 1H), 8.39 (d, J=7.3 Hz, 1H), 8.36-8.32 (m, 1H), 8.28 (d, J=5.2 Hz, 1H), 7.52 (d, J=7.5 Hz, 1H), 7.26 (d, J=5.2 Hz, 1H), 7.18 (d, J=8.6 Hz, 1H), 7.05 (t, J=7.5 Hz, 1H), 6.98 (d, J=6.9 Hz, 1H), 3.17 (s, 2H), 3.04 (s, 3H), 2.86 (s, 2H), 2.59 (s, 4H), 2.52 (s, 6H), 2.19 (s, 3H).

### Procedure for the Synthesis of 553, 554 and 555

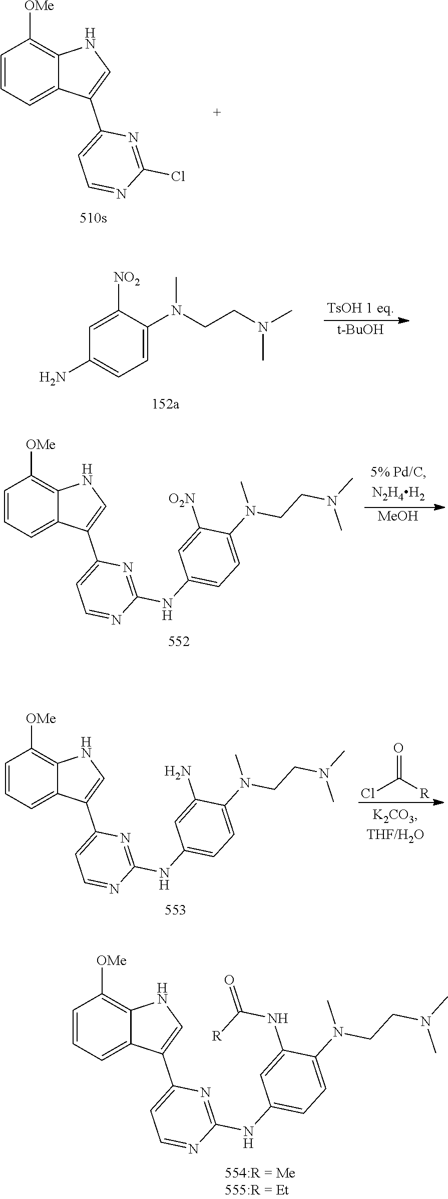

### N¹-(2-(dimethylamino)ethyl)-N⁴-(4-(7-methoxy-1H-indol-3-yl)pyrimidin-2-yl)-N¹-methyl-2-nitrobenzene-1,4-diamine (552)

To the mixture of 510s (153 mg, 0.59 mmol) and 152a (142 mg, 0.59 mmol) in t-BuOH (10 mL) were added p-Toluenesulfonic acid monohydrate (447 mg, 2.35 mmol). The mixture was stirred at 85° C. for 16 h. After cooling to room temperature, the mixture was concentrated and the residue was purified by flash-chromatography (dichloromethane/methanol=100/1-10/1) to give 552 (127 mg, 0.275 mmol, yield 46.6%) as a brown solid. MS-ESI (M+H)⁺: 462.25

### N¹-(2-(dimethylamino)ethyl)-N⁴-(4-(7-methoxy-1H-indol-3-yl)pyrimidin-2-yl)-N¹-methylbenzene-1,2,4-triamine (553)

A mixture of 552 (127 mg, 0.275 mmol) and Pd/C (5%, 50 mg) in methanol (20 mL) was stirred at 50-60° C. under H₂ for 16 h. After concentration under vacuum, the residue was triturated with ethyl acetate (200 mL) and filtered. The filtrate was concentrated under reduced pressure to give crude title product 553 (50 mg), which was used to next step without further purification.

### N-(2-((2-(dimethylamino)ethyl)(methyl)amino)-5-((4-(7-methoxy-1H-indol-3-yl)pyrimidin-2-yl)amino)phenyl)acetamide (554)

A mixture of 553 (50 mg) and K₂CO₃ (30 mg) in tetrahydrofuran (2 mL) and water (0.1 ml) was stirred at 0° C. for half an hour. Acetyl chloride (25 mg) was added dropwise to the mixture at 0° C. The mixture was stirred at 25° C. for 1 h. The reaction was quenched with saturated NH₄Cl (2 mL) and concentrated in vacuo. The residue was dissolved with water (10 mL) and extracted with dichloromethane (10 mL×3). The organic phase was washed with brine (10 mL×3), dried by Na₂SO₄, filtered and concentrated. The residue was purified by preparative thin layer chromatography (dichloromethane/methanol=10/1) to give 554 (4 mg) as an off-white solid. MS-ESI (M+H⁺): 475.0.

### N-(2-((2-(dimethylamino)ethyl)(methyl)amino)-5-((4-(7-methoxy-1H-indol-3-yl)pyrimidin-2-yl)amino)phenyl)propionamide (555)

A mixture of 553 (105 mg) and K₂CO₃ (50 mg) in tetrahydrofuran (5 mL) and water (0.2 ml) was stirred at 0° C. for half an hour. Propionyl chloride (32 mg) was added dropwise to the mixture at 0° C. The mixture was stirred at 25° C. for 1 h. The reaction was quenched with saturated NH₄Cl (2 mL) and concentrated in vacuo. The residue was dissolved with water (10 mL) and extracted with dichloromethane (10 mL×3). The organic phase was washed with brine (10 mL×3), dried by Na₂SO₄, filtered and concentrated. The residue was purified by preparative thin layer chromatography (dichloromethane/methanol=10/1) to give 555 (25.3 mg) as an off-white solid. MS-ESI (M+H⁺): 488.9.

### Material and Methods

### Cell Culture and Reagents

MV-4-11 and H1975 cells were obtained from the American Type Culture Collection and KM12-luc cells were obtained from JCRB Cell Bank. All cell lines were maintained at 37° C. with 5% C02, in media supplemented with 10% fetal bovine serum and penicillin (100 units/mL) and streptomycin (100 μg/mL). MV-4-11 cells were maintained in Iscove's Modified Dubecco's Medium, H1975 cells were cultured with RPMI 1640 media, and KM12-luc cells were maintained in Dulbecco's Modification of Eagle's Medium. CCK-8 reagent was obtained from Yeasen.

### Cell Proliferation and Growth Inhibition Assay

Cell proliferation was assessed by CCK-8 assay (Yeasen) per the manufacturer's instructions. The MV-4-11 (AML), H1975, and KM12-luc cells were seeded at 30,000, 3,000 and 2,500 cells per well onto 96-well plates, and after a 24 h incubation, the cells were treated with test compounds for 72 hrs. Cell viability was assayed by incubating the cells with CCK-8 reagent for 1-4 hrs. Absorbance was measured at OD450 using the Beckman DTX880. The data was calculated using GraphPad Prism version 6.01. The IC50 values were fitted using a non-linear regression model with a sigmoidal dose response.

TABLE 1
Results of biological testing

| | AML | NTRK | EGFR |
|---|---|---|---|
| | IC50 in MV-4-11 | IC50 in KM12-luc | IC50 in H1975 |
| Cmpd ID# | (nM) | (nM) | (nM) |
| 156 | NA | 370.2 | 2795.0 |
| 158 | NA | >1000 | NA |
| 160 | NA | 209.7 | NA |
| 161 | NA | 146.5 | NA |
| 162 | NA | 129.1 | NA |
| 164 | NA | 50.5 | NA |
| 165 | NA | 170.8 | NA |
| 166 | 96.8 | 39.5 | 691.1 |
| 169 | 25.9 | 43.8 | 285.2 |
| 173 | NA | 85.7 | NA |
| 177 | NA | 131.5 | NA |
| 180 | NA | 3064.0 | NA |
| 184 | NA | 407.3 | NA |
| 189 | NA | 28.7 | NA |
| 190 | NA | 60.4 | NA |
| 192 | NA | 525.3 | NA |
| 194 | NA | 722.0 | NA |
| 197 | NA | 375.8 | NA |
| 201 | NA | 443.9 | 1100.0 |
| 204 | NA | 45.8 | NA |
| 206 | NA | 175.8 | NA |
| 207 | NA | 348.0 | NA |
| 208 | NA | 381.1 | NA |
| 211 | NA | 40.3 | NA |
| 216 | NA | 49.7 | NA |
| 218 | NA | 103.4 | NA |
| 222 | NA | 1435.0 | NA |
| 237 | NA | 536.9 | NA |
| 239 | NA | 517.9 | NA |
| 244 | NA | 348.3 | NA |
| 246 | NA | 69.9 | NA |
| 253 | NA | 97.5 | NA |
| 260 | NA | 145.3 | NA |
| 261 | NA | 509.0 | NA |
| 262 | NA | 966.0 | NA |
| 263 | NA | 704.3 | NA |
| 264 | NA | 1039.0 | NA |
| 265 | NA | 413.7 | NA |
| 266 | NA | 763.7 | NA |
| 267 | NA | 302.9 | NA |
| 268 | NA | 784.1 | NA |
| 269 | NA | 111.7 | NA |
| 270 | NA | 1605.0 | NA |
| 271 | NA | 1477.0 | NA |
| 274 | NA | 128.0 | NA |
| 275 | NA | 600.0 | NA |
| 276 | NA | 679.2 | NA |
| 277 | NA | 368.0 | NA |
| 278 | NA | 17.1 | NA |
| 279 | NA | 52.6 | NA |
| 283 | NA | 86.4 | NA |
| 284 | 63.8 | 15.0 | 1033.0 |
| 285 | NA | 99.6 | NA |
| 286 | NA | 14.6 | NA |
| 287 | NA | 21.9 | NA |
| 289 | NA | >10000 | NA |
| 290 | NA | >10000 | NA |
| 294 | NA | 167.0 | NA |
| 295 | NA | 158.9 | NA |
| 296 | NA | 10.7 | 1073.0 |
| 297 | NA | 37.7 | NA |
| 298 | NA | 44.0 | 288.9 |
| 301 | NA | 75.4 | NA |
| 302 | NA | 96.8 | NA |
| 304 | NA | 75.1 | NA |
| 305 | NA | 28.9 | 653.0 |
| 306 | NA | 56.1 | NA |
| 307 | NA | 386.3 | NA |
| 308 | NA | 138.6 | NA |
| 309 | NA | 134.7 | NA |
| 310 | NA | 304.9 | NA |
| 313 | NA | 307.6 | NA |
| 315 | NA | 85.1 | NA |
| 316 | NA | 21.6 | 476.6 |
| 317 | NA | 24.9 | NA |
| 318 | NA | 43.2 | NA |
| 319 | 29.7 | 19.1 | 772.2 |
| 320 | NA | 91.1 | NA |
| 321 | NA | 14.8 | NA |
| 322 | NA | 8.3 | NA |
| 323 | NA | 18.9 | NA |
| 324 | NA | 17.5 | NA |
| 325 | NA | 39.4 | NA |
| 326 | NA | 58.2 | NA |
| 327 | NA | 260.2 | NA |
| 329 | NA | 72.1 | NA |
| 332 | NA | 126.7 | NA |
| 333 | NA | 28.5 | NA |
| 334 | 3.4 | 3.0 | 92.1 |
| 335 | 3.1 | 6.1 | 75.4 |
| 336 | NA | 64.8 | NA |
| 337 | 11.1 | 10.4 | 366.0 |
| 339 | NA | 75.2 | NA |
| 340 | NA | 57.4 | NA |
| 341 | NA | 248.2 | NA |
| 342 | NA | 24.6 | NA |
| 343 | 2.6 | 4.3 | 77.3 |
| 346 | NA | 105.2 | NA |
| 347 | NA | 114.4 | NA |
| 348 | NA | 87.8 | NA |
| 349 | NA | 245.3 | NA |
| 350 | NA | 126.2 | NA |
| 351 | NA | 172.3 | NA |
| 352 | NA | 42.1 | NA |
| 355 | NA | 70.4 | NA |
| 356 | NA | 326.9 | NA |
| 357 | NA | 1075.0 | NA |
| 358 | 4.4 | 3.1 | 47.0 |
| 359 | 5.9 | 5.5 | 120.3 |
| 362 | NA | 32.6 | 18.6 |
| 363 | NA | 93.4 | NA |
| 364 | NA | 20.1 | NA |
| 365 | 4.4 | 4.3 | 68.3 |
| 368 | NA | 22.9 | 356.2 |
| 369 | NA | 27.6 | NA |
| 370 | NA | 77.1 | NA |
| 371 | NA | 110.4 | NA |
| 372 | NA | 30.2 | NA |
| 374 | 5.3 | 3.7 | 52.5 |
| 375 | 4.2 | 4.1 | 151.6 |
| 378 | NA | 43.4 | 26.1 |
| 379 | NA | 148.8 | NA |
| 380 | NA | 50.5 | NA |
| 381 | 5.2 | 7.2 | 167.7 |
| 384 | NA | 26.3 | 506.1 |
| 385 | NA | 45.3 | NA |
| 386 | NA | 43.9 | NA |
| 387 | NA | 114.4 | NA |
| 388 | NA | 44.8 | NA |
| 391 | NA | 50.0 | NA |
| 392 | NA | 18.1 | 861.5 |
| 393 | NA | 23.1 | NA |
| 395 | NA | 77.3 | NA |
| 396 | NA | 80.3 | NA |
| 397 | NA | 180.4 | NA |
| 398 | NA | 196.1 | NA |
| 401 | NA | 49.5 | NA |
| 402 | 64.2 | 27.4 | 1207.0 |
| 403 | NA | 46.5 | NA |
| 404 | NA | 26.5 | NA |
| 405 | NA | >1000 | NA |
| 406 | NA | 60.1 | NA |
| 407 | NA | 44.4 | NA |
| 408 | NA | 19.7 | NA |
| 411 | NA | 12.8 | NA |
| 412 | NA | 8.4 | 380.0 |
| 417 | NA | 94.2 | NA |
| 418 | NA | 79.2 | NA |
| 422 | 5.2 | 5.2 | 287.6 |
| 423 | NA | 4.7 | 66.5 |
| 428 | NA | 46.4 | NA |
| 429 | NA | 475.3 | NA |
| 433 | NA | 13.9 | 506.1 |
| 434 | NA | 97.1 | NA |
| 437 | NA | 108.8 | NA |
| 440 | NA | 7.0 | NA |
| 441 | NA | 24.9 | NA |
| 442 | NA | 6.4 | 327.6 |
| 443 | NA | 33.3 | NA |
| 444 | 2.9 | 5.5 | 299.0 |
| 446 | 10.6 | 4.9 | 667.1 |
| 448 | NA | >1000 | NA |
| 449 | NA | >1000 | NA |
| 451 | NA | 14.2 | NA |
| 452 | >1,000 | NA | >20,000 |
| 491 | NA | >3333 | NA |
| 493 | NA | >10000 | NA |
| 494 | NA | >1000 | NA |
| 497 | NA | 4033.0 | NA |
| 500 | NA | 3573.0 | NA |
| 501 | NA | >1000 | NA |
| 503 | NA | 3639.0 | NA |
| 505 | NA | >1000 | NA |
| 507 | NA | >1000 | NA |
| 508 | NA | >1000 | NA |
| 513 | NA | NA | NA |
| 514 | NA | NA | NA |
| 518 | NA | NA | 405.7 |
| 520 | NA | NA | 1177.0 |
| 521 | NA | NA | 694.6 |
| 522 | NA | NA | 998.8 |
| 524 | NA | NA | 424.7 |
| 525 | NA | NA | 405.7 |
| 526 | NA | NA | 656.0 |
| 528 | NA | NA | 297.7 |
| 529 | NA | NA | 958.3 |
| 530 | NA | NA | 769.3 |
| 532 | NA | NA | 791.2 |
| 533 | NA | NA | 1106.0 |
| 534 | NA | NA | 1298.0 |
| 536 | NA | NA | 837.0 |
| 537 | NA | NA | 1081.0 |
| 538 | NA | NA | 3683.0 |
| 540 | NA | NA | 95.4 |
| 542 | NA | NA | 80.4 |
| 543 | NA | NA | 67.4 |
| 545 | NA | NA | 876.5 |
| 546 | NA | NA | 1025.0 |
| 547 | NA | NA | 1176.0 |
| 549 | NA | NA | 681.7 |
| 550 | NA | NA | 1511.0 |
| 551 | NA | NA | 1539.0 |
| 553 | NA | NA | 1135.0 |
| 554 | NA | NA | 1034.0 |
| 555 | NA | NA | 2254.0 |
| 181a | NA | >10000 | NA |
| 181b | NA | >10000 | NA |
| 195a | NA | 152.7 | NA |
| 195b | NA | 561.2 | NA |
| 205a | NA | 37.0 | NA |
| 205b | NA | 303.8 | NA |
| 205c | NA | 329.1 | NA |
| 212a | NA | 62.7 | NA |
| 212b | NA | 37.0 | 1661.0 |
| 212c | NA | 43.2 | NA |
| 212d | NA | 93.1 | NA |
| 212e | NA | 3359.8 | NA |
| 212f | NA | 546.1 | NA |
| 227a | NA | 578.1 | NA |
| 227b | NA | 671.6 | NA |
| 227c | NA | 435.5 | NA |
| 227d | NA | 2011.0 | NA |
| 232a | NA | 85.5 | NA |
| 232b | NA | 491.9 | NA |
| 248a | NA | 12.8 | 441.6 |
| 248b | NA | 17.1 | 1049.0 |
| 255a | NA | 246.2 | NA |
| 255b | NA | >10000 | NA |
| 256a | NA | 446.7 | NA |
| 256b | NA | 747.5 | NA |
| 256c | NA | 586.7 | NA |
| 256d | NA | 1001.0 | NA |
| 257a | NA | >10000 | NA |
| 257b | NA | >10000 | NA |
| 257c | NA | >10000 | NA |
| 257d | NA | >10000 | NA |
| 258a | NA | >1000 | NA |
| 258b | NA | >1000 | NA |
| 258c | NA | >1000 | NA |
| 258d | NA | >1000 | NA |
| 259a | NA | 65.7 | NA |
| 259b | NA | 541.1 | NA |
| 272a | NA | 41.0 | NA |
| 272b | NA | 2061.0 | NA |
| 273a | NA | 127.0 | NA |
| 273b | NA | 3320.0 | NA |
| 291a | NA | 8665.0 | NA |
| 291b | NA | 1402.0 | NA |
| 291c | NA | 2050.0 | NA |
| 291d | NA | 3266.0 | NA |
| 299a | NA | 48.1 | NA |
| 299b | NA | 3376.0 | NA |
| 300a | NA | 27.9 | NA |
| 300b | NA | 3681.0 | NA |
| 303a | NA | 19.1 | 108.9 |
| 303b | NA | 64.3 | NA |
| 314a | NA | 63.2 | NA |
| 314b | NA | 138.7 | NA |
| 328a | NA | 34.1 | NA |
| 328b | NA | 2424.0 | NA |
| 330a | NA | 291.6 | NA |
| 330b | NA | >3333 | NA |
| 331a | NA | 32.3 | NA |
| 331b | NA | 2021.0 | NA |
| 394a | NA | 29.1 | NA |
| 394b | NA | 131.2 | NA |
| 400a | NA | 42.0 | 1006.0 |
| 400b | NA | 71.5 | NA |
| R-280 | NA | 15.6 | NA |
| R-281 | NA | 120.7 | NA |
| R-288 | NA | 15.5 | NA |
| R-344 | 5.4 | 7.4 | 205.0 |
| R-345 | NA | 6.2 | NA |
| R-353 | NA | 64.3 | NA |
| R-354 | NA | 69.1 | NA |
| R-366 | 5.3 | 5.2 | 88.6 |
| R-367 | NA | 5.7 | NA |
| R-373 | NA | 22.8 | 922.7 |
| R-382 | 6.3 | 9.2 | 128.5 |
| R-383 | 4.7 | 8.5 | 129.4 |
| R-389 | NA | 24.8 | 939.8 |
| R-414 | NA | 12.4 | NA |
| R-415 | NA | 66.7 | NA |
| R-420 | NA | 72.0 | NA |
| R-425 | NA | 11.3 | NA |
| R-426 | 3.0 | 2.1 | 152.3 |
| R-431 | NA | 31.8 | NA |
| R-432 | NA | 129.6 | NA |
| R-435 | NA | 31.9 | 1039.0 |
| R-439 | NA | 127.5 | NA |
| S-280 | NA | 14.6 | 1254.0 |
| S-288 | NA | 16.9 | 1255.0 |
| S-344 | 5.6 | 6.3 | 198.3 |
| S-345 | NA | 8.2 | NA |
| S-353 | NA | 35.2 | NA |
| S-354 | NA | 56.3 | NA |
| S-366 | 3.0 | 3.1 | 97.8 |
| S-367 | NA | 4.6 | NA |
| S-373 | NA | 21.1 | 460.7 |
| S-382a | 3.7 | 5.3 | 134.2 |
| S-382b | NA | 348.8 | NA |
| S-383 | 4.6 | 9.5 | 117.9 |
| S-389 | NA | 23.5 | 897.7 |
| S-390 | NA | 44.6 | NA |
| S-409 | NA | 24.6 | NA |
| S-414 | NA | 50.1 | NA |
| S-415 | NA | 155.1 | NA |
| S-420 | NA | 260.7 | NA |
| S-425 | 3.2 | 3.5 | 167.1 |
| S-426 | 4.2 | 2.7 | 188.8 |
| S-431 | NA | 39.6 | NA |
| S-432 | NA | 22.4 | NA |
| S-435 | NA | 48.1 | 1564.0 |
| S-439 | NA | 150.0 | NA |

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

1. (canceled)

2. A compound of Formula (I): 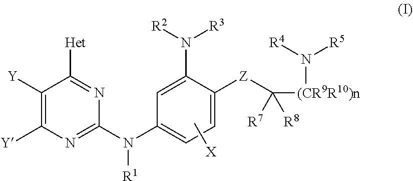
or an enantiomer, a mixture of enantiomers, a mixture of two or more diastereomers, a tautomer, a mixture of two or more tautomers, or an isotopic variant thereof, or a pharmaceutically acceptable salt thereof, wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Z is selected from O, NR⁶, C(═O), SO₂, C(═O)NR⁶, NR⁶C(═O), SO₂NR⁶, and (CH₂)₁₋₂, or
when Z is NR⁶, NR³ and R⁶ taken together form a fused imidazolyl ring;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl;
R³ is selected from H, C₁-C₃ alkyl, C₁-C₃ haloalkyl, and —C(O)—R¹¹,
wherein R¹¹ is selected from H, C₁-C₃ alkyl, C₁-C₃ alkoxy, and C₁-C₃ haloalkyl or
R⁴ is selected from H and C₁-C₃ alkyl;
R⁵ is selected from H, C₁-C₃ alkyl, C₁-C₃ haloalkyl, and —C(O)—R¹²,
wherein R¹² is selected from H, C₁-C₃ alkyl, C₁-C₃ alkoxy, and C₁-C₃ haloalkyl;
R⁶ is selected from H, C₁-C₃ alkyl, and C(O)—R¹³,
wherein R¹³ is selected from H, C₁-C₃ alkyl, C₁-C₃ alkoxy, and C₁-C₃ haloalkyl;
R⁷ and R⁸ are each independently selected from H and C₁-C₃ alkyl; or R⁷ and R⁸ taken together can represent oxo (═O);
R⁹ and R¹⁰ are each independently selected from H and C₁-C₃ alkyl;
n is 1 to 4.

3. The compound of claim 2, or a pharmaceutically acceptable salt thereof, wherein Y′ is H.

4. The compound of claim 2, or a pharmaceutically acceptable salt thereof, wherein Y is selected from F, CF₃, OMe and Cl.

5. The compound of claim 2, or a pharmaceutically acceptable salt thereof, wherein Het is selected from 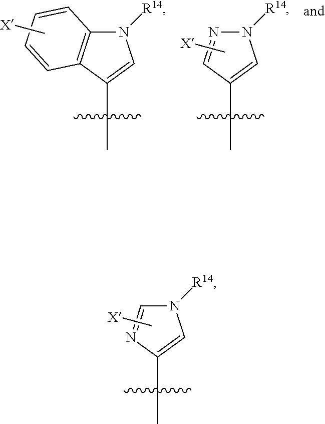
wherein each X′ represents up to two optional substituents independently selected from halo, C₁-C₃ alkyl, and C₁-C₃ alkoxy, and each R¹⁴ represents H, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy.

6. The compound of claim 2, or a pharmaceutically acceptable salt thereof, wherein Z is NR⁶.

7. The compound of claim 2, or a pharmaceutically acceptable salt thereof, wherein n is 1.

8. The compound of nm claim 2, or a pharmaceutically acceptable salt thereof, wherein R⁷, R⁸, R⁹ and R¹⁰ each represent H.

9. The compound of nm claim 2, or a pharmaceutically acceptable salt thereof, wherein R¹ is H.

10. The compound of claim 2, or a pharmaceutically acceptable salt thereof, wherein R² is H.

11. The compound of claim 2, which is a compound of Formula (IA): 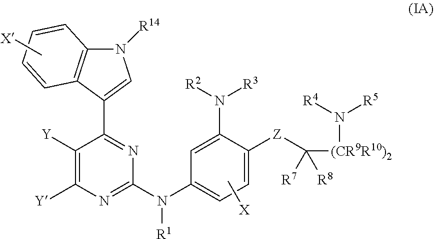
or an enantiomer, a mixture of enantiomers, a mixture of two or more diastereomers, a tautomer, a mixture of two or more tautomers, or an isotopic variant thereof; or a pharmaceutically acceptable salt thereof,
wherein R¹⁴ is selected from H, methyl, ethyl, isopropyl, cyclopropyl and —C(═O)—R* where R* is H, C₁₋₃ haloalkyl, or C₁₋₄ alkyl optionally substituted with OH, CN, or C₁₋₃ alkoxy.

12. The compound of claim 2, which is of the formula (IB): 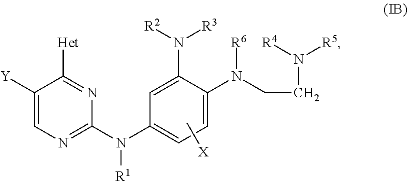
or an enantiomer, a mixture of enantiomers, a mixture of two or more diastereomers, a tautomer, a mixture of two or more tautomers, or an isotopic variant thereof; or a pharmaceutically acceptable salt thereof;
wherein R⁴, R⁵, and R⁶ are each independently selected from methyl and ethyl.

13. 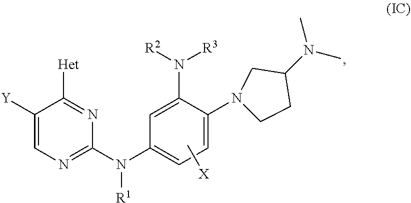
or an enantiomer, a mixture of enantiomers, a mixture of two or more diastereomers, a tautomer, a mixture of two or more tautomers, or an isotopic variant thereof, or a pharmaceutically acceptable salt thereof, wherein
X represents one or two optional substituents independently selected from halo, C₁-C₃ alkyl, C₁-C₃ alkoxy, and C₁-C₃ haloalkyl;
Y is selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy; C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R² is selected from H and C₁-C₃ alkyl; and
R³ is selected from H, C₁-C₃ alkyl, C₁-C₃ haloalkyl, and —C(O)—R¹¹,
wherein R¹¹ is selected from H, C₁-C₃ alkyl, C₁-C₃ alkoxy, and C₁-C₃ haloalkyl.

14. A compound of Formula (ID): 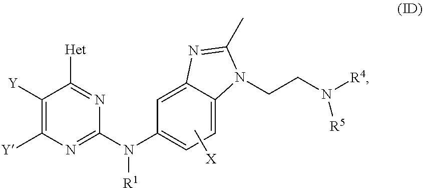
or an enantiomer, a mixture of enantiomers, a mixture of two or more diastereomers, a tautomer, a mixture of two or more tautomers, or an isotopic variant thereof; or a pharmaceutically acceptable salt thereof, wherein:
X represents one or two optional substituents independently selected from halo, C₁-C₃ alkyl, C₁-C₃ alkoxy, and C₁-C₃ haloalkyl;
Y and Y′ each independently represent a group selected from H, halo, C₁-C₃ alkyl, C₁-C₃ alkoxy, C₁-C₃ haloalkyl, and amino;
Het represents a 5-9 atom heteroaromatic monocyclic or bicyclic group comprising at least one heteroatom selected from N, O and S as a ring member, and Het is optionally substituted with one to three groups independently selected from halo, C₁-C₃ alkoxy, C₁-C₃ haloalkyl, C₁₋₄ alkyl optionally substituted with one or more groups selected from halo, OH, CN, C₁₋₃ alkoxy and C₁₋₃ haloalkoxy, C₃₋₆ cycloalkyl optionally substituted with one or more groups selected from C₁₋₃ alkyl, halo, OH, CN, C₁₋₃ alkoxy and C₁₋₃ haloalkoxy, or —C(═O)—R* where R* is H, C₁₋₃ haloalkyl, or C₁₋₄ alkyl optionally substituted with OH, CN, or C₁₋₃ alkoxy, or Het is an optionally substituted indole;
R¹ is selected from H and C₁-C₃ alkyl;
R⁴ is selected from H and C₁-C₃ alkyl; and
R⁵ is selected from H, C₁-C₃ alkyl, C₁-C₃ haloalkyl, and —C(O)—R¹²,
wherein R¹² is selected from H, C₁-C₃ alkyl, C₁-C₃ alkoxy, and C₁-C₃ haloalkyl.

15. The compound of claim 2, or a pharmaceutically acceptable salt thereof, wherein Z is O.

16. A compound of Formula (II): 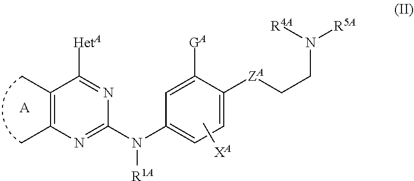
or an enantiomer, a mixture of enantiomers, a mixture of two or more diastereomers, a tautomer, a mixture of two or more tautomers, or an isotopic variant thereof, or a pharmaceutically acceptable salt thereof, wherein:
Ring A is an optional 5-6 membered heterocyclic ring fused to the pyrimidine in Formula (II), comprising one or two heteroatoms selected from N and O as ring members,
wherein Ring A can be aromatic or non-aromatic and is optionally substituted with one or two groups independently selected from halo, C₁-C₃ alkoxy, C₁-C₃ haloalkyl, and C₁-C₃ alkyl;
Alternatively, Ring A is absent, and pyrimidine is optionally substituted with one or two groups independently selected from halo, C₁-C₃ alkoxy, C₁-C₃ haloalkyl, and C₁-C₃ alkyl;
Het^{A} represents a 5-9 atom heteroaromatic monocyclic or bicyclic group comprising at least one heteroatom selected from N, O and S as a ring member, and Het^{A} is optionally substituted with one to three groups independently selected from halo, C₁-C₃ alkoxy, C₁-C₃ haloalkyl, and C₁-C₃ alkyl;
X^{A} represents one or two optional substituents independently selected from halo, C₁-C₃ alkyl, C₁-C₃ alkoxy, and C₁-C₃ haloalkyl;
R^{1A} is selected from H and C₁-C₃ alkyl;
G^{A} is selected from —NR^{2A}R^{3A}, SO₂R^{7A}, halo, and C₁-C₃ haloalkyl;
wherein R^{2A} is selected from H and C₁-C₃ alkyl, and
R^{3A} is selected from H, C₁-C₃ alkyl, C₂-C₄ alkenyl, C₁-C₃ haloalkyl, —SO₂R^{7A}, and —C(O)—R^{11A},
wherein R^{11A} is selected from H, C₁-C₃ alkyl, C₂-C₄ alkenyl, C₁-C₃ alkoxy, and C₁-C₃ haloalkyl;
Z^{A} is selected from O, NR^{6A}, C(═O), SO₂, C(═O)NR^{6A}, SO₂NR^{6A}, and (CH₂)₁₋₂;
R^{4A} is selected from H and C₁-C₃ alkyl;
R^{5A} is selected from H, C₁-C₃ alkyl, C₁-C₃ haloalkyl, —C(O)—R^{12A}, and —SO₂R^{7A},
wherein R^{12A} is selected from H, C₁-C₃ alkyl, C₁-C₃ alkoxy, and C₁-C₃ haloalkyl;
R^{6A} is selected from H and C₁-C₃ alkyl; and
each R^{7A} is independently C₁-C₃ alkyl.

17. (canceled)

18. (canceled)

19. (canceled)

20. (canceled)

21. (canceled)

22. (canceled)

23. (canceled)

24. A compound of Formula (III): 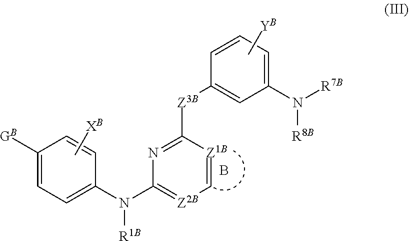
or an enantiomer, a mixture of enantiomers, a mixture of two or more diastereomers, a tautomer, a mixture of two or more tautomers, or an isotopic variant thereof, or a pharmaceutically acceptable salt thereof, wherein:
Ring B is an optional 5 membered heteroaromatic ring fused to the ring containing Z^{2B} in Formula (III), comprising N or O as a ring member,
wherein Ring B is optionally substituted with one or two groups independently
selected from halo, C₁-C₃ alkoxy, C₁-C₃ haloalkyl, C₁-C₃ alkyl, and —C(O)—R^{11B};
Z^{1B} is N when ring B is absent, and Z^{1B} is C when ring B is present;
Z^{2B} is N when ring B is present, and Z^{2B} is CR^{2B} when ring B is absent;
Z^{3B} is NR^{3B} or O;
G^{B} is a group of the formula —NR^{4B}—(CR^{1B})₂₋₃—NR^{5B}R^{6B} or G^{B} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10B};
X^{B} represents one or two optional substituents independently selected from halo, C₁-C₃ alkyl, C₁-C₃ alkoxy, and C₁-C₃ haloalkyl;
Y^{B} represents one or two optional substituents independently selected from halo, C₁-C₃ alkyl, C₁-C₃ alkoxy, and C₁-C₃ haloalkyl;
R^{1B} is selected from H and C₁-C₃ alkyl;
R^{2B} is selected from H, halo, C₁-C₃ alkyl, and C₁-C₃ haloalkyl;
R^{3B} is selected from H and C₁-C₃ alkyl;
R^{4B} is selected from H, C₁-C₃ alkyl, and —C(O)—R^{10B};
R^{5B} is selected from H and C₁-C₃ alkyl;
R^{6B} is selected from H, C₁-C₃ alkyl, and —C(O)—R^{10B};
R^{7B} is selected from H and C₁-C₃ alkyl;
R^{8B} is selected from H, C₁-C₃ alkyl and —C(O)—R^{10B};
each R^{10B} is independently selected from H, C₁-C₃ alkyl, C₁-C₃ alkoxy, and C₁-C₃ haloalkyl.

25. (canceled)

26. (canceled)

27. (canceled)

28. (canceled)

29. (canceled)

30. (canceled)

31. (canceled)

32. (canceled)

33. A compound of Formula (IV): 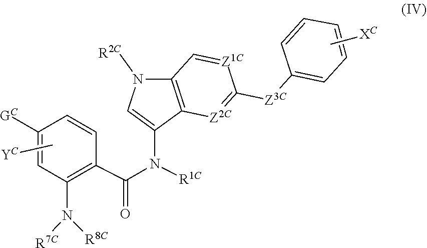
or an enantiomer, a mixture of enantiomers, a mixture of two or more diastereomers, a tautomer, a mixture of two or more tautomers, or an isotopic variant thereof, or a pharmaceutically acceptable salt thereof, wherein:
Z^{1C} and Z^{2C} are independently selected from N and CH;
Z^{3C} is selected from O, CH₂, and NR^{3C};
G^{C} is a group of the formula —NR^{4C}—(CR^{2C})₂₋₃—NR^{5C}R^{6C} or G^{C} is a 5-6 membered saturated ring comprising one or two nitrogen atoms as ring members, which is optionally substituted with one or two groups independently selected from halo, C₁-C₃ alkoxy, C₁-C₃ haloalkyl, C₁-C₃ alkyl, and —C(O)—R^{10C};
X^{C} represents one or two optional substituents independently selected from halo, C₁-C₃ alkyl, C₁-C₃ alkoxy, and C₁-C₃ haloalkyl;
Y^{C} represents one or two optional substituents independently selected from halo, C₁-C₃ alkyl, C₁-C₃ alkoxy, and C₁-C₃ haloalkyl;
R^{1C} is selected from H and C₁-C₃ alkyl;
R^{2C} is selected from H and C₁-C₃ alkyl;
R^{3C} is selected from H and C₁-C₃ alkyl;
R^{4C} is selected from H, C₁-C₃ alkyl, and —C(O)—R^{10C};
R^{5C} is selected from H and C₁-C₃ alkyl;
R^{6C} is selected from H, C₁-C₃ alkyl, and —C(O)—R^{10C};
R^{7C} is selected from H, C₁-C₄ alkyl optionally substituted with C₁-C₃ alkoxy, and a 5-6 membered heterocyclic group containing a heteroatom selected from N, O and S as a ring member, and optionally substituted with one or two groups independently selected from halo, C₁-C₃ alkyl, C₁-C₃ alkoxy, and C₁-C₃ haloalkyl;
R^{8C} is selected from H, C₁-C₃ alkyl and —C(O)—R^{10C};
each R^{10C} is independently selected from H, C₁-C₃ alkyl, C₁-C₃ alkoxy, and C₁-C₃ haloalkyl.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. A pharmaceutical composition comprising a compound of claim 2, or a pharmaceutically acceptable salt thereof, and at least one pharmaceutically acceptable excipient.

51. A pharmaceutical combination comprising a compound of claim 2, or a pharmaceutically acceptable salt thereof, and an additional therapeutic agent, wherein the additional therapeutic agent is one suitable for treating the same condition(s) as the compound of Formula (I).

52. A method to treat a cancer, which comprises administering to a subject in need of such treatment a compound of claim 2, or a pharmaceutically acceptable salt thereof.

53. The method of claim 52, wherein the cancer is associated with a kinase selected from FLT3, EGFR, VEGFR, ALK, NTRK, RET, ROS/ROS1, DYRK1 and CK2a kinases.

54. The method of claim 52, wherein the cancer is acute myeloid leukemia (AML), hepatocellular carcinoma (HCC), thyroid cancer, mast cell tumors (MCT), solid tumors with an NTRK gene fusion, leukemia, lymphoma, lung cancer, colon and colorectal cancer, CNS cancer, melanoma, ovarian cancer, renal cancer, prostate cancer, breast cancer, liver cancer, sarcoma, epidermoid cancer, fibrosarcoma, cervical cancer, gastric carcinoma, skin cancer, head and neck cancers, or pancreatic cancer.

55. (canceled)

56. (canceled)

57. (canceled)

58. (canceled)

59. (canceled)

60. (canceled)
